(19) United States
(12) Patent Application Publication
(10) Pub. No.: US 2024/0199613 A1
(43) Pub. Date: Jun. 20, 2024

(54) POLYCYCLIC INHIBITORS OF PLASMA KALLIKREIN

(71) Applicant: TAKEDA PHARMACEUTICAL COMPANY LIMITED, Osaka-shi Osaka (JP)

(72) Inventors: Nikolaos PAPAIOANNOU; Jeremy Mark TRAVINS, Lexington, MA (US); Sarah Jocelyn FINK; John Mark ELLARD, Saffron Walden (GB); Alastair RAE, Saffron Walden (GB); Stuart Shane RANKIN, Saffron Walden (GB); Robert Staurt Laurie CHAPMAN, Saffron Walden (GB)

(21) Appl. No.: 18/550,486

(22) PCT Filed: Mar. 16, 2022
(86) PCT No.: PCT/US2022/020527

§ 371 Date: Sep. 14, 2023

Related U.S. Application Data
(60) Provisional application No. 63/162,483, filed on Mar. 17, 2021.

(51) Int. Cl. C07D 471/04; C07D 519/00

(52) U.S. Cl. CPC C07D 471/04; C07D 519/00

(57) ABSTRACT

The present invention provides compounds and compositions thereof which are useful as inhibitors of plasma kallikrein and which exhibit desirable characteristics for the same.

### CROSS REFERENCE TO RELATED APPLICATIONS

This application claims the benefit of U.S. Provisional Application No. 63/162,483, filed Mar. 17, 2021, which is herein incorporated by reference in its entirety.

### I. BACKGROUND OF THE INVENTION

Plasma Kallikrein (PKa) is a serine protease zymogen in blood that is converted to its catalytically active form by coagulation factor XIIa, and contributes to the innate inflammatory response and intrinsic cascade of blood coagulation. The mechanisms that lead to the activation of this pathway in vivo include interactions with polyphosphates released from activated platelets and deficiency of C1 inhibitor (C1-INH), the primary physiological inhibitor of PKa. PKa-mediated cleavage of high-molecular weight kininogen generates the potent vasodilator and pro-inflammatory nonapeptide bradykinin (BK), which activates the bradykinin 2 receptor. Subsequent cleavage of BK by carboxypeptidases generates des-Arg9-BK, which activates the B1 receptor. Both B1 and B2 receptors are expressed by vascular, glial, and neuronal cell types, with the highest levels of retinal expression detected in the ganglion cell layer and inner and outer nuclear layers. Activation of B1 and B2 receptors causes vasodilation and increases vascular permeability.

PKa is also associated with a number of disorders, such as hereditary angioedema (HAE), an autosomal dominant disease characterized by painful, unpredictable, recurrent attacks of inflammation affecting the hands, feet, face, abdomen, urogenital tract, and the larynx. Prevalence for HAE is uncertain but is estimated to be approximately 1 case per 50,000 persons without known differences among ethnic groups. HAE is caused by deficient (Type I) or dysfunctional (Type II) levels of C1-INH, which inhibits PKa, bradykinin, and other serine proteases in the blood. Individuals with hereditary angioedema (HAE) are deficient in C1-INH and consequently undergo excessive bradykinin generation, which in turn cause painful, debilitating, and potentially fatal swelling attacks. If left untreated, HAE can result in a mortality rate as high as 40% primarily due to upper airway obstruction.

### II. SUMMARY OF THE INVENTION

The present disclosure is based on, at least in part, the development of a number of compounds which bind to plasma kallikrein and effectively inhibit its activity. Accordingly, provided herein are compounds and uses thereof for targeting plasma kallikrein and/or treating plasma kallikrein-mediated diseases and disorders, novel intermediates, and processes for preparing compounds disclosed herein. The disclosure also extends to pharmaceutical compositions comprising any one of the same, and use of compounds or compositions herein for treatment, in particular treatment of autoimmune disease, such as HAE.

In some embodiments, the present invention provides a compound of Formula (I):

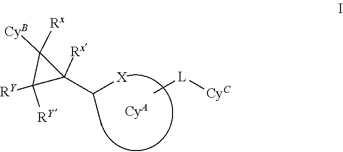

or a pharmaceutically acceptable salt thereof, wherein each of Cy^{A}, X, Cy^{B}, Cy^{C}, L, R^{x}, R^{x′}, R^{Y}, and R^{Y′} is defined and described in classes and subclasses herein, both singly and in combination. In certain embodiments, the present invention provides compounds of Formulae (I)-(VI-c), as defined and described in classes and subclasses herein.

In some embodiments, the present invention also provides methods of using compounds of Formulae (I)-(VI-c).

Advantageously, compounds of the present disclosure have therapeutic activity and adequate levels of bioavailability and/or adequate half-life for use as a therapeutic.

### III. DETAILED DESCRIPTION OF CERTAIN EMBODIMENTS

### A. Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The abbreviations used herein have their conventional meaning within the chemical and biological arts. The chemical structures and formulae set forth herein are constructed according to the standard rules of chemical valency known in the chemical arts.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocyclyl,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In some embodiments, aliphatic groups contain 1-4 aliphatic carbon atoms. In some embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocyclyl” or “cycloalkyl”) refers to a monocyclic C₃-C₇ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “halogen” means F, Cl, Br, or I.

The term “aryl” refers to monocyclic and bicyclic ring systems having a total of five to 10 ring members, wherein at least one ring in the system is aromatic and wherein each ring in the system contains three to seven ring members. The term “aryl” may be used interchangeably with the term “aryl ring”. In some embodiments, an 8-10 membered bicyclic aryl group is an optionally substituted naphthyl ring. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-” refer to groups having 5 to 10 ring atoms, preferably 5, 6, or 9 ring atoms; having 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (or in the case of a bivalent fused heteroarylene ring system, at least one radical or point of attachment is on a heteroaromatic ring). Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted.

As used herein, the terms “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in this context in reference to a ring atom, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As used herein and unless otherwise specified, the suffix “-ene” is used to describe a bivalent group. Thus, any of the terms above can be modified with the suffix “-ene” to describe a bivalent version of that moiety. For example, a bivalent carbocycle is “carbocycylene”, a bivalent aryl ring is “arylene”, a bivalent benzene ring is “phenylene”, a bivalent heterocycle is “heterocyclylene”, a bivalent heteroaryl ring is “heteroarylene”, a bivalent alkyl chain is “alkylene”, a bivalent alkenyl chain is “alkenylene”, a bivalent alkynyl chain is “alkynylene”, and so forth.

As described herein, compounds of the invention may, when specified, contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

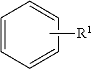

refers to at least

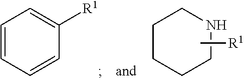

refers to at least

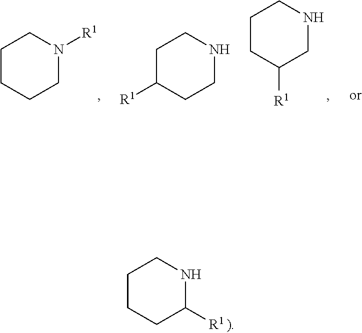

In addition, in a polycyclic ring system, substituents may, unless otherwise indicated, replace a hydrogen on any individual ring (e.g.,

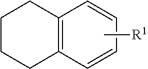

refers to at least

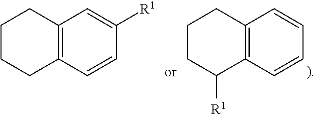

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O(CH₂)₀₋₄C(O)OR^{∘}; —O(CH₂)₀₋₄OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)_{0- 4}C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6 membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12 membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR^{#}₂, ═NNHC(O)R^{#}, ═NNHC(O)OR^{#}, ═NNHS(O)₂R^{#}, ═NR^{#}, ═NOR^{#}, —O(C(R^{#}₂))₂₋₃O—, or —S(C(R^{#}₂))₂₋₃S—, wherein each independent occurrence of R is selected from hydrogen, C₁₋₆ aliphatic which may be substituted as defined below, or an unsubstituted 5-6 membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{#}₂)₂₋₃O—, wherein each independent occurrence of R^{#} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{#} include halogen, —R^{•}, -(haloR•), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6 membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R, —S(O)₂NR₂, —C(S)NR^{†}₂, —C(NH)NR^{†}₂, or —N(R^{†})S(O)₂R^{†}; wherein each R is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12 membered saturated, partially unsaturated, or aryl mono- or bicyclic ring having 0-4 heteroatoms independently selected from nitrogen, oxygen, or sulfur.

Suitable substituents on the aliphatic group of R are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference.

In certain embodiments, the neutral forms of the compounds are regenerated by contacting the salt with a base or acid and isolating the parent compound in the conventional manner. In some embodiments, the parent form of the compound differs from the various salt forms in certain physical properties, such as solubility in polar solvents.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In some embodiments, compounds of the present disclosure are provided as a single enantiomer or single diastereoisomer. Single enantiomer refers to an enantiomeric excess of 80% or more, such as 81, 82, 83, 84, 85, 86, 87, 88, 89, 90, 91, 92, 93, 94, 95, 96, 97, 98 or 99%. Single diastereoisomer excess refers to an excess of 80% or more, for example 81, 82, 83, 84, 85, 86, 87, 88, 89, 90, 91, 92, 93, 94, 95, 96, 97, 98 or 99%.

The term “oxo,” as used herein, means an oxygen that is double bonded to a carbon atom, thereby forming a carbonyl.

The symbol “ [symbol] ”, except when used as a bond to depict unknown or mixed stereochemistry, denotes the point of attachment of a chemical moiety to the remainder of a molecule or chemical formula.

The articles “a” and “an” are used herein to refer to one or to more than one (i.e., to at least one) of the grammatical object of the article. By way of example, “an element” means one element or more than one element.

A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses.

As will be understood from context, a “reference” compound is one that is sufficiently similar to a particular compound of interest to permit a relevant comparison. In some embodiments, information about a reference compound is obtained simultaneously with information about a particular compound. In some embodiments, information about a reference compound is historical. In some embodiments, information about a reference compound is stored, for example in a computer-readable medium. In some embodiments, comparison of a particular compound of interest with a reference compound establishes identity with, similarity to, or difference of the particular compound of interest relative to the compound.

As used herein, the phrase “therapeutic agent” refers to any agent that has a therapeutic effect and/or elicits a desired biological and/or pharmacological effect, when administered to a subject.

As used herein, the term “therapeutically effective amount” refers to an amount of a therapeutic agent that confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic agent effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific therapeutic agent employed; the specific composition employed; the age, body weight, general health, sex and diet of the subject; the time of administration, route of administration, and/or rate of excretion or metabolism of the specific therapeutic agent employed; the duration of the treatment; and like factors as is well known in the medical arts.

As used herein, the term “treatment” (also “treat” or “treating”) refers to any administration of a substance (e.g., provided compositions) that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### B. Compounds

In some embodiments, a provided compound is of formula (I):

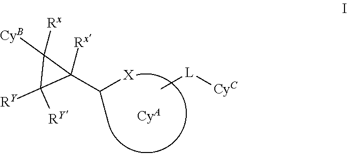

or a pharmaceutically acceptable salt thereof,
wherein:
Cy^{A} is an 8- to 10-membered bicyclic heteroarylene having 1-5 heteroatoms independently selected from oxygen, nitrogen, and sulfur, a 10- to 14-membered tricyclic heteroarylene having 1-6 heteroatoms independently selected from oxygen, nitrogen, and sulfur, an 8- to 14-membered saturated or partially unsaturated bicyclic heterocyclyl having 1-6 heteroatoms selected from oxygen, nitrogen, or sulfur, or a 10- to 15-membered saturated or partially unsaturated tricyclic heterocyclyl having 1-6 heteroatoms selected from oxygen, nitrogen, or sulfur, wherein Cy^{A} is substituted with 0-6 —R^{A} groups;
   each R^{A} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, 5- to 6-membered heteroaryl having 1-4 heteroatoms independently selected from oxygen, nitrogen, and sulfur, 3- to 7-membered saturated or partially unsaturated monocyclic carbocyclyl, 3- to 7-membered saturated or partially unsaturated monocyclic heterocyclyl having 1-2 heteroatoms selected from oxygen, nitrogen, or sulfur, or a 6- to 12-membered spirocyclic ring system having 0-4 heteroatoms independently selected from oxygen, nitrogen, and sulfur;
each R is independently hydrogen or an optionally substituted group selected from C₁₋₆ aliphatic, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur;
X is —N═ or —NR—, wherein X is adjacent to the ring atom of Cy^{A} attached to the cyclopropyl ring; each R^{Y} and R^{Y′} is independently selected from hydrogen, halogen, and an optionally substituted C₁₋₆ aliphatic group;
each R^{x} and R^{x′} is independently selected from hydrogen, halogen, or —CN;
Cy^{B} is selected from phenyl, 8- to 10-membered bicyclic aryl, a 5- to 6-membered heteroaryl having 1-3 heteroatoms independently selected from oxygen, nitrogen, and sulfur, or a 7- to 10-membered heteroaryl having 1-4 heteroatoms independently selected from oxygen, nitrogen, and sulfur, wherein Cy^{B} is substituted with 0-5 —R^{B} groups; or
Cy^{B} and R^{x}, together with their intervening atoms, form a 6- to 12-membered spirocyclic ring system having 0-4 heteroatoms independently selected from oxygen, nitrogen, and sulfur, wherein the ring or rings formed by Cy^{B} and R^{x} may be substituted with 0-4 —R^{B} groups;
   each R^{B} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, a 3- to 7-membered saturated or partially unsaturated monocyclic heterocyclyl having 1-2 heteroatoms selected from oxygen, nitrogen, or sulfur, or a 5- to 6-membered heteroaryl having 1-4 heteroatoms independently selected from oxygen, nitrogen, and sulfur;
L is an optionally substituted C₁₋₃ hydrocarbon chain, wherein 1 to 3 methylene units are optionally and independently replaced with —O—, —NR^{z}—, —S—, —SO—, or —SO₂—; or L is an optionally substituted 5- to 6-membered saturated or partially unsaturated heterocyclene, having 1-3 heteroatoms independently selected from oxygen, nitrogen, and sulfur;
   each R^{z} is independently selected from hydrogen, —(CH₂)₀₋₃OR, —(CH₂)₀₋₃C(O)OR, or an optionally substituted C₁₋₆ aliphatic group;
Cy^{C} is a 7- to 10-membered bicyclic heteroaryl having 1-4 heteroatoms independently selected from oxygen, nitrogen, and sulfur, or a 6- to 12-membered saturated or partially unsaturated bicyclic heterocyclyl having 1-4 heteroatoms independently selected from oxygen, nitrogen, or sulfur, wherein Cy^{C} is substituted with 0-6 -L^{C}-R^{C} groups;
   each L^{C} is independently selected from a covalent bond or an optionally substituted C₁₋₆ hydrocarbon chain, wherein 1 to 3 methylene units are optionally and independently replaced with —O— or —NR—;
   each R^{C} is independently selected from halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, Cy^{D}, or an optionally substituted group selected from C₁₋₆ aliphatic;
   each Cy^{D} is independently selected from a 3- to 7-membered saturated or partially unsaturated monocyclic carbocyclyl, a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, phenyl, a 6- to 12-membered saturated or partially unsaturated bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur, wherein Cy^{D} is substituted with 0-4 -L^{D}-R^{D} groups;
      each L^{D} is independently selected from a covalent bond or an optionally substituted C₁₋₆ hydrocarbon chain, wherein 1 to 3 methylene units are optionally and independently replaced with —O— or —NR—; and
      each R^{D} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, a 3- to 7-membered saturated or partially unsaturated monocyclic carbocyclyl, or a 3- to 7-membered saturated or partially unsaturated monocyclic heterocyclyl having 1-2 heteroatoms selected from oxygen, nitrogen, or sulfur.

It will be appreciated that, “oxo” refers a double bonded oxygen substitution on a carbon “C═O”, where the carbon atom is part of the structure or group that is substituted by oxo. For example, where Cy^{C} is substituted with -L^{D}-R^{D}, and where L^{D} is a covalent bond and R^{D} is oxo, the carbon atom substituted with oxo (i.e., the carbon in C═O) is part of Cy^{C} (e.g., a structure of Cy^{C} being cyclopentyl substituted with -L^{D}-R^{D} at the 2-position, where L^{D} is a covalent bond and R^{D} is oxo corresponds to

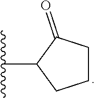 [SMILES: *C1CCCC1=O]

It will be appreciated that for Formula I, the reference to “the ring atom of Cy^{A} attached to the cyclopropyl ring” refers to the ring atom marked below with “*”

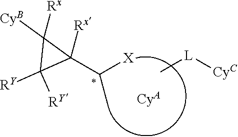

In some embodiments, Cy^{A} is an 8- to 10-membered bicyclic heteroarylene having 1-5 heteroatoms independently selected from oxygen, nitrogen, and sulfur, wherein Cy^{A} is substituted with 0-4 —R^{A} groups. In certain embodiments, Cy^{A} is quinolinylene substituted with 0-4 —R^{A} groups.

In some embodiments, Cy^{A} is a 10- to 14-membered tricyclic heteroarylene having 1-6 heteroatoms independently selected from oxygen, nitrogen, and sulfur, wherein Cy^{A} is substituted with 0-5 —R^{A} groups.

In some embodiments, Cy^{A} is selected from the group consisting of:

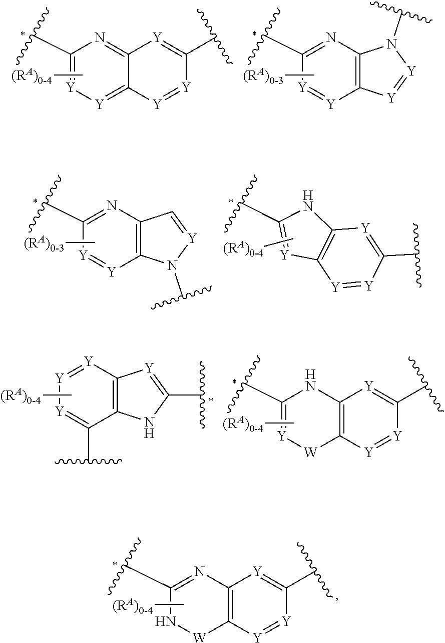

wherein:
each Y is independently selected from ═C(H)— or ═N—;
W is selected from —C(O)— or —S(O)₂—;
V is selected from ═CH— or ═N—;
and * represents point of attachment to the cyclopropyl ring.

In some embodiments, Cy^{A} is selected from the group consisting of

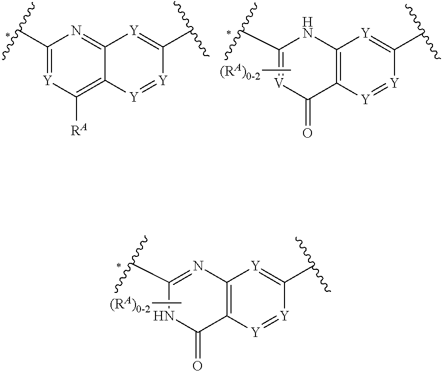

wherein:
* represents point of attachment to the cyclopropyl ring.

In some embodiments, Cy^{A} is selected from the group consisting of:

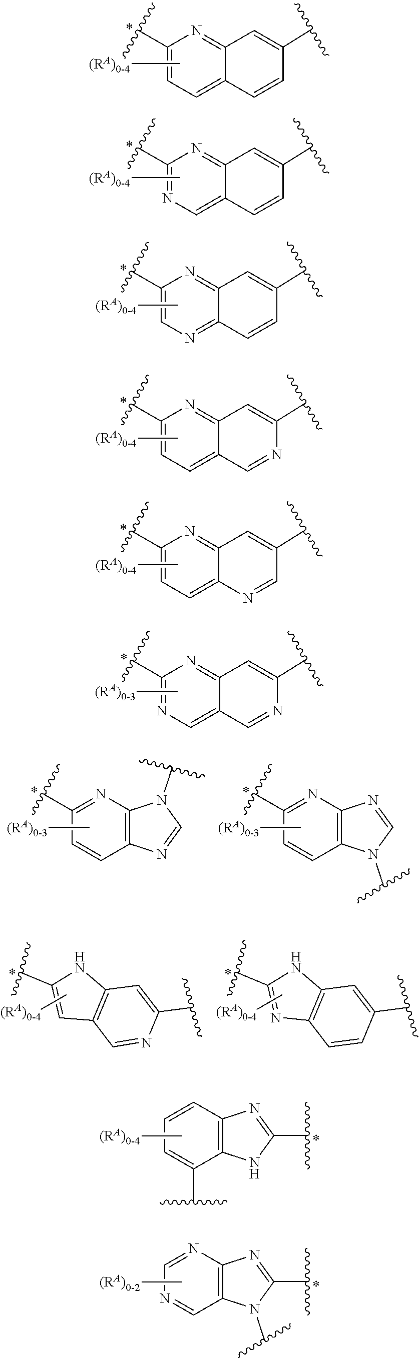

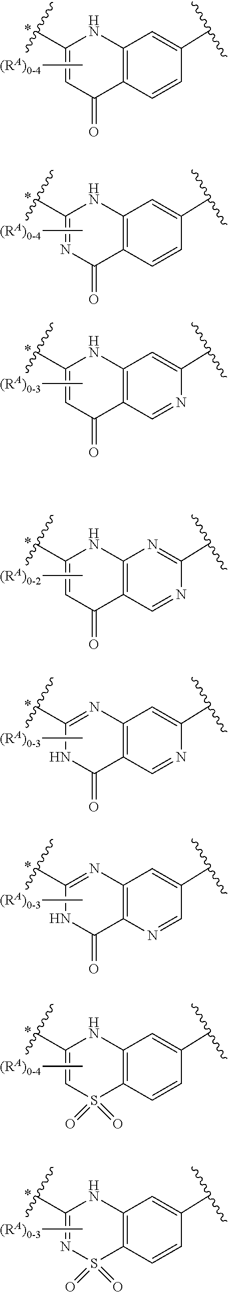

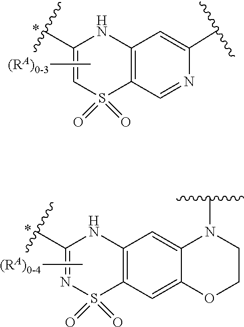

wherein * represents point of attachment to the cyclopropyl ring.

In some embodiments, Cy^{A} is selected from the group consisting of:

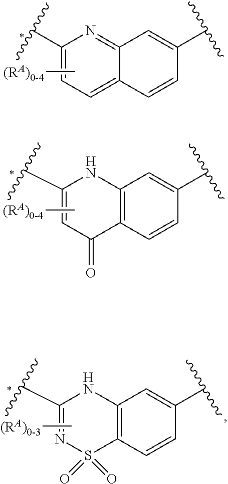

wherein * represents point of attachment to the cyclopropyl ring.

In some embodiments, Cy^{A} is selected from the group consisting of:

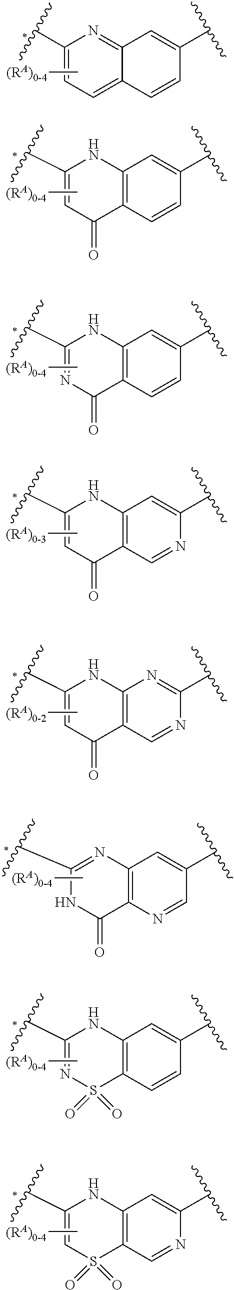

wherein * represents point of attachment to the cyclopropyl ring.

In some embodiments, Cy^{A} is selected from the group consisting of:

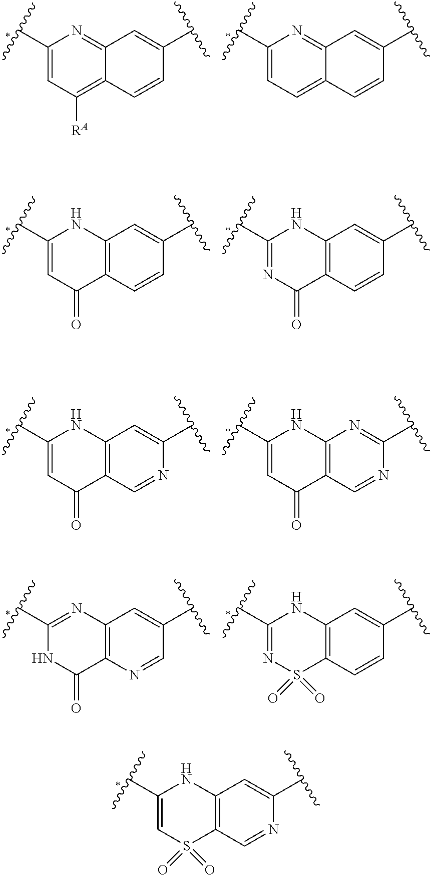

wherein * represents point of attachment to the cyclopropyl ring.

In some embodiments, each R^{A} is independently selected from oxo, halogen, —CN, —N(R)₂, —N(R)S(O)₂R, —OR, or an optionally substituted group selected from C₁₋₆ aliphatic, 5- to 6-membered heteroaryl having 1-4 heteroatoms independently selected from oxygen, nitrogen, and sulfur, 3- to 7-membered saturated or partially unsaturated monocyclic heterocyclyl having 1-2 heteroatoms selected from oxygen, nitrogen, or sulfur, or a 6- to 12-membered spirocyclic ring system having 0-4 heteroatoms independently selected from oxygen, nitrogen, and sulfur.

In some embodiments, a single instance of R^{A} is oxo. In some embodiments, a single instance of R^{A} is halogen. In some embodiments, a single instance of R^{A} is —CN. In some embodiments, a single instance of R^{A} is —N(R)₂. In some embodiments, a single instance of R^{A} is —N(R)₂, wherein each R is independently hydrogen or an optionally substituted C₁₋₆ aliphatic group. In some embodiments, a single instance of R^{A} is —N(R)S(O)₂R. In some embodiments, a single instance of R^{A} is —N(R)S(O)₂R, wherein each R is an optionally substituted C₁₋₆ aliphatic group. It will be appreciated that references herein to embodiments in which “a single instance” of a substituent is defined are not limited to monosubstituted embodiments. For example, “[i]n some embodiments, a single instance of R^{A} is oxo” includes embodiments in which at least one instance of R^{A} is oxo and which may comprise one or more additional R^{A} groups as defined herein.

In some embodiments, a single instance of R^{A} is —OR. In some embodiments, a single instance of R^{A} is —OR, wherein R is selected from hydrogen or an optionally substituted group selected from C₁₋₆ aliphatic or 3- to 7-membered saturated or partially unsaturated monocyclic heterocyclyl having 1-2 heteroatoms selected from oxygen, nitrogen, or sulfur. In some embodiments, a single instance of R^{A} is —OR, wherein R is oxetanyl. In some embodiments, a single instance of R^{A} is —OR, wherein R is piperidinyl.

In some embodiments, a single instance of R^{A} is an optionally substituted C₁₋₆ aliphatic group.

In some embodiments, a single instance of R^{A} is an optionally substituted 5- to 6-membered heteroaryl having 1-4 heteroatoms independently selected from oxygen, nitrogen, and sulfur. In some embodiments, a single instance of R^{A} is imidazolyl. In some embodiments, a single instance of R^{A} is pyrazolyl. In some embodiments, a single instance of R^{A} is triazolyl.

In some embodiments, a single instance of R^{A} is an optionally substituted 3- to 7-membered saturated or partially unsaturated monocyclic heterocyclyl having 1-2 heteroatoms selected from oxygen, nitrogen, or sulfur. In some embodiments, a single instance of R^{A} is azetidinyl. In some embodiments, a single instance of R^{A} is piperazinyl. In some embodiments, a single instance of R^{A} is morpholinyl. In some embodiments, a single instance of R^{A} is thiomorpholinyl.

In some embodiments, a single instance of R^{A} is an optionally substituted 6- to 12-membered spirocyclic ring system having 0-4 heteroatoms independently selected from oxygen, nitrogen, and sulfur. In some embodiments, a single instance of R^{A} is:

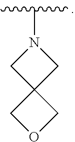 [SMILES: *N1CC2(COC2)C1]

In some embodiments, substituents on an optionally substituted R^{A} group are independently —(CH₂)₀₋₄R^{∘}, —(CH₂)₀₋₄OR^{∘}, or —CN, wherein each R^{∘} is independently as defined above and described in classes and subclasses herein.

In some embodiments, X is —N═. In some embodiments, X is —NR—. In some embodiments, X is —NH—.

In some embodiments, Cy^{B} is selected from phenyl, a 5- to 6-membered heteroaryl having 1-3 heteroatoms independently selected from oxygen, nitrogen, and sulfur or a 7- to 10-membered heteroaryl having 1-4 heteroatoms independently selected from oxygen, nitrogen, and sulfur, wherein Cy^{B} is substituted with 0-4 —R^{B} groups.

In some embodiments, Cy^{B} is selected from phenyl or a 5- to 6-membered heteroaryl having 1-3 heteroatoms independently selected from oxygen, nitrogen, and sulfur, wherein Cy^{B} is substituted with 0-4 —R^{B} groups. In some embodiments, Cy^{B} is selected from phenyl or a 6-membered heteroaryl having 1-3 heteroatoms independently selected from oxygen, nitrogen, and sulfur, wherein Cy^{B} is substituted with 0-4 —R^{B} groups.

In some embodiments, Cy^{B} is phenyl, wherein Cy^{B} is substituted with 0-4 —R^{B} groups. In some embodiments, Cy^{B} is phenyl, wherein Cy^{B} is substituted with 0-3 —R^{B} groups.

In some embodiments, Cy^{B} is a 6-membered heteroaryl having 1-3 heteroatoms independently selected from oxygen, nitrogen, and sulfur, wherein Cy^{B} is substituted with 0-4 —R^{B} groups. In some embodiments, Cy^{B} is a 6-membered heteroaryl having 1-3 nitrogens, wherein Cy^{B} is substituted with 0-4 —R^{B} groups. In some embodiments, Cy^{B} is a pyrimidinyl group substituted with 0-2 —R^{B} groups. In some embodiments, Cy^{B} is a pyridinyl group substituted with 0-2 —R^{B} groups. In some embodiments, Cy^{B} is a pyrazinyl group substituted with 0-1 —R^{B} groups. In some embodiments, Cy^{B} is a pyridazinyl group substituted with 0-1 —R^{B} groups. In some embodiments, Cy^{B} is a 1,3,5-triazinyl group substituted with 0-1 —R^{B} groups.

In some embodiments, Cy^{B} is a 5-membered heteroaryl having 1-3 heteroatoms independently selected from oxygen, nitrogen, and sulfur, wherein Cy^{B} is substituted with 0-4 —R^{B} groups. In some embodiments, Cy^{B} is a 5-membered heteroaryl having 1-2 heteroatoms independently selected from sulfur and nitrogen, wherein Cy^{B} is substituted with 0-4 —R^{B} groups. In some embodiments, Cy^{B} is a thienyl group substituted with 0-2 —R^{B} groups. In some embodiments, Cy^{B} is a thiazolyl group substituted with 0-1 —R^{B} groups. In some embodiments, Cy^{B} is a thiadiazolyl group substituted with 0-1 —R^{B} groups.

In some embodiments, Cy^{B} is selected from the group consisting of:

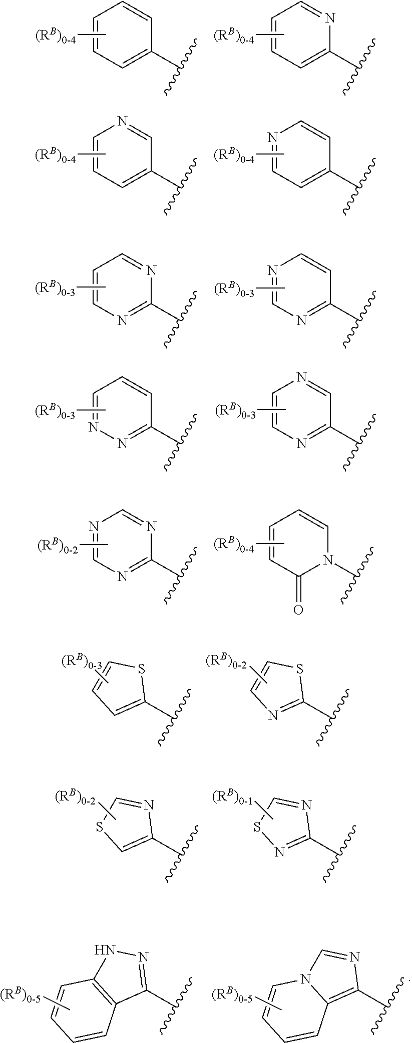

In some embodiments, Cy^{B} is selected from the group of:

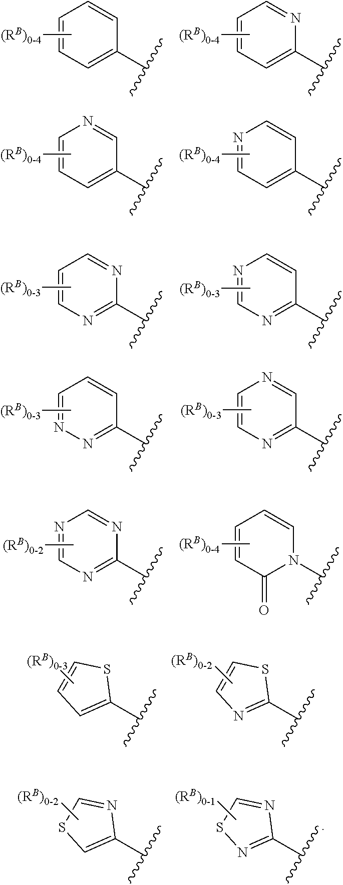

In some embodiments, Cy^{B} is selected from the group consisting of:

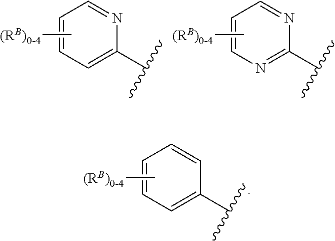

In some embodiments, Cy^{B} and R^{x}, together With their intervening atoms, form a 6- to 12-membered spirocyclic ring system having 0-4 heteroatoms independently selected from oxygen, nitrogen, and sulfur, wherein the ring or rings formed by Cy^{B} and R^{x} may be substituted with 0-4 —R^{B} groups. It will be appreciated that references herein to the number of atoms in a spirocyclic ring system (e.g., 6- to 12-membered) include the depicted cyclopropyl ring.

In some embodiments, Cy^{B} and R^{x}, together with their intervening atoms, form a 6- to 12-membered spirocyclic ring system having 0-1 nitrogen heteroatoms, wherein the ring or rings formed by Cy^{B} and R^{x} may be substituted with 1-3 —R^{B} groups.

In some embodiments, Cy^{B} and R^{x}, together with their intervening atoms, form a 6- to 12-membered spirocyclic ring system selected from:

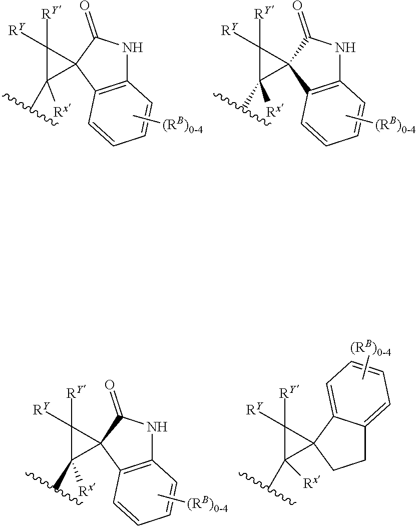

In some embodiments, each R^{B} is independently selected from oxo, halogen, —CN, —NO₂, —N(R)₂, —N(R)C(O)₂R, —OR, or an optionally substituted group selected from C₁₋₆ aliphatic or a 5-membered heteroaryl having 1-4 heteroatoms independently selected from oxygen, nitrogen, and sulfur.

In some embodiments, substituents on an optionally substituted R^{B} group are independently selected from oxo, halogen, and —(CH₂)₀₋₄OR^{∘}, wherein each R^{∘} is independently as defined above and described in classes and subclasses herein.

In some embodiments, a single instance of R^{B} is oxo. In some embodiments, a single instance of R^{B} is halogen. In some embodiments, a single instance of R^{B} is chloro. In some embodiments, a single instance of R^{B} is —CN. In some embodiments, a single instance of R^{B} is —NO₂. In some embodiments, a single instance of R^{B} is —N(R)₂, In some embodiments, a single instance of R^{B} is —N(R)C(O)₂R. In some embodiments, a single instance of R^{B} is —OR.

In some embodiments, a single instance of R^{B} is optionally substituted C₁₋₆ aliphatic. In some embodiments, a single instance of R^{B} is C₁₋₆ aliphatic substituted with halogen. In some embodiments, a single instance of R^{B} is methyl.

In some embodiments, a single instance of R^{B} is —N(R)C(O)₂R, wherein each R is independently selected from hydrogen or C₁₋₆ aliphatic optionally substituted with —(CH₂)₀₋₄R^{∘}, wherein each R^{∘} is independently as defined above and described in classes and subclasses herein.

In some embodiments, a single instance of R^{B} is —OR, wherein each R is independently selected from hydrogen or C₁₋₆ aliphatic optionally substituted with halogen, —(CH₂)₀₋₄OR^{∘}, or (CH₂)₀₋₄C(O)OR^{∘}, wherein each R^{∘} is independently as defined above and described in classes and subclasses herein.

In some embodiments, a single instance of R^{B} is a 5-membered heteroaryl having 1-4 heteroatoms independently selected from oxygen, nitrogen, and sulfur. In some embodiments, a single instance of R^{B} is tetrazolyl.

In some embodiments, each of R^{x} and R^{x′} is independently selected from hydrogen and halogen. In some embodiments, each of R^{x} and R^{x′} is hydrogen. In some embodiments, one of R^{x} and R^{x′} is hydrogen and the other is halogen.

In some embodiments, each of R^{Y} and R^{Y′} is independently selected from hydrogen and halogen.

In some embodiments, each of R^{Y} and R^{Y′} is hydrogen.

In some embodiments, R^{Y} is an optionally substituted C₁₋₆ aliphatic group and R^{Y′} is hydrogen. In some embodiments, R^{Y} is substituted with —(CH₂)₀₋₄OR^{∘}, wherein R^{∘} is as defined above and described in classes and subclasses herein.

In some embodiments, L is an optionally substituted C₁₋₃ hydrocarbon chain, wherein 1-3 methylene units are optionally replaced with —O—, —NR^{z}—, —S—, or —SO₂—. In some embodiments, L is an optionally substituted C₁₋₃ hydrocarbon chain, wherein 1 methylene unit is optionally replaced with —O—, —NR^{z}—, —S—, or —SO₂—.

In some embodiments, L is an optionally substituted C₁ hydrocarbon chain.

In some embodiments, L is an optionally substituted C₁ hydrocarbon chain, wherein the 1 methylene unit is replaced with 5-membered saturated or partially unsaturated heterocyclene having 1 nitrogen heteroatom, optionally substituted with —(CH₂)₀₋₄OR^{∘}, wherein R^{∘} is as defined above and described in classes and subclasses herein.

In some embodiments, L is —CH₂—. In some embodiments, L is optionally substituted

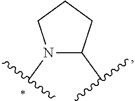 [SMILES: *C1CCCN1*]

wherein * represents the point of attachment to Cy^{A}. In some embodiments, L is optionally substituted

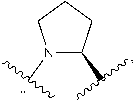 [SMILES: *[C@@H]1CCCN1*]

wherein * represents the point of attachment to Cy^{A}. In some embodiments, L is optionally substituted

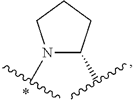 [SMILES: *[C@H]1CCCN1*]

wherein * represents the point of attachment to Cy^{A}. In some embodiments, L is

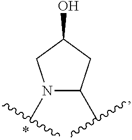 [SMILES: *C1C[C@H](O)CN1*]

wherein * represents the point of attachment to Cy^{A}. In some embodiments, L is

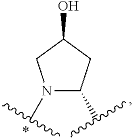 [SMILES: *[C@H]1C[C@H](O)CN1*]

wherein * represents the point of attachment to Cy^{A}. In some embodiments, L is

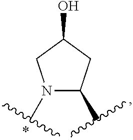 [SMILES: *[C@@H]1C[C@H](O)CN1*]

wherein * represents the point of attachment to Cy^{A}.

In some embodiments, L is an optionally substituted C₂ hydrocarbon chain, wherein 1 methylene unit is optionally replaced with —NR^{z}— or —O—. In some embodiments, L is an optionally substituted C₂ hydrocarbon chain, wherein the methylene unit connected to Cy^{A} is replaced with —NR^{z}- or —O—. In some embodiments, L is an optionally substituted C₂ hydrocarbon chain, wherein the methylene unit connected to Cy^{A} is replaced with —NR^{z}—. In some embodiments, L is an optionally substituted C₂ hydrocarbon chain, wherein the methylene unit connected to Cy^{A} is replaced with —NR^{z}—, and wherein R^{z} is selected from hydrogen, —(CH₂)₀₋₃C(O)OR, or an optionally substituted C₁₋₆ aliphatic group.

In some embodiments, L is an optionally substituted C₂ hydrocarbon chain, wherein the methylene unit connected to Cy^{A} is replaced with —O—.

In some embodiments, L is

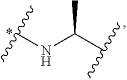 [SMILES: *N[C@H](*)C]

wherein * represents the point of attachment to Cy^{A}. In some embodiments, L is

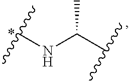 [SMILES: *N[C@@H](*)C]

wherein * represents the point of attachment to Cy^{A}. In some embodiments, L is *—NHCH₂—, wherein * represents the point of attachment to Cy^{A}. In some embodiments, L is *—N(CH₃)CH₂—, wherein * represents the point of attachment to Cy^{A}. In some embodiments, L is

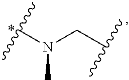

wherein * represents the point of attachment to Cy^{A}. In some embodiments, L is

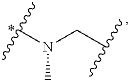

wherein * represents the point of attachment to Cy^{A}. In some embodiments, L is *—OCH(Me)-, wherein * represents the point of attachment to Cy^{A}. In some embodiments, L is *—OCH₂—, wherein * represents the point of attachment to Cy^{A}.

In some embodiments, L comprises a two-atom spacer between Cy^{A} and Cy^{C}.

In some embodiments, L is an optionally substituted 5- to 6-membered saturated or partially unsaturated heterocyclene, having 1-3 heteroatoms independently selected from oxygen, nitrogen, and sulfur. In some embodiments, L is an optionally substituted 5-membered saturated or partially unsaturated heterocyclene, having 1 heteroatom independently selected from oxygen, nitrogen, and sulfur. In some embodiments, L is an optionally substituted pyrrolidinediyl group. In some embodiments, L is optionally substituted

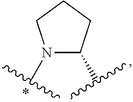 [SMILES: *[C@H]1CCCN1*]

wherein * represents the point of attachment to Cy^{A}.

In some embodiments, optional substituents on L are independently selected from —(CH₂)₀₋₄R^{∘}, —(CH₂)₀₋₄OR^{∘}, —(CH₂)₀₋₄OC(O)R^{∘}, and —(CH₂)₀₋₄N(R^{∘})₂, wherein each R^{∘} is independently as defined above and described in classes and subclasses herein.

In some embodiments, Cy^{C} is an 8- to 10-membered bicyclic aryl, wherein Cy^{C} is substituted with 0-6-L^{C}-R^{C} groups. In some embodiments, Cy^{C} is quinolinyl, substituted with 0-6-L^{C}-R^{C} groups.

In some embodiments, Cy^{C} is an 8- to 10-membered heteroaryl having 1-4 heteroatoms independently selected from oxygen, nitrogen, and sulfur, wherein Cy^{C} is substituted with 0-6-L^{C}-R^{C} groups. In some embodiments, Cy^{C} is a 9- to 10-membered heteroaryl having 1-4 heteroatoms independently selected from oxygen, nitrogen, and sulfur, wherein Cy^{C} is substituted with 0-6-L^{C}-R^{C} groups. In some embodiments, Cy^{C} is a 9-membered heteroaryl having 1-4 nitrogen heteroatoms, wherein Cy^{C} is substituted with 0-6-L^{C}-R^{C} groups. In some embodiments, Cy^{C} is a 9-membered heteroaryl having 1 nitrogen and 1 sulfur heteroatoms, wherein Cy^{C} is substituted with 0-6-L^{C}-R^{C} groups. In some embodiments, Cy^{C} is a 10-membered heteroaryl having 1-4 heteroatoms independently selected from oxygen, nitrogen, and sulfur, wherein Cy^{C} is substituted with 0-6-L^{C}-R^{C} groups. In some embodiments, Cy^{C} is a 10-membered heteroaryl having 1 nitrogen heteroatom, wherein Cy^{C} is substituted with 0-6-L^{C}-R^{C} groups. In some embodiments, Cy^{C} is a 9-membered heteroaryl having 2 nitrogen heteroatoms, wherein Cy^{C} is substituted with 0-6-L^{C}-R^{C} groups.

In some embodiments, Cy^{C} is triazolopyridinyl, wherein Cy^{C} is substituted with 0-4 -L^{C}-R^{C} groups. In some embodiments, Cy^{C} is pyrazolopyridinyl, wherein Cy^{C} is substituted with 0-5 -L^{C}-R^{C} groups. In some embodiments, Cy^{C} is pyrazolopyrimidinyl, wherein Cy^{C} is substituted with 0-4 -L^{C}-R^{C} groups. In some embodiments, Cy^{C} is triazolopyridazinyl, wherein Cy^{C} is substituted with 0-3 -L^{C}-R^{C} groups. In some embodiments, Cy^{C} is imidazopyridazinyl, wherein Cy^{C} is substituted with 0-4 -L^{C}-R^{C} groups. In some embodiments, Cy^{C} is imidazopyrimidinyl, wherein Cy^{C} is substituted with 0-4 -L^{C}-R^{C} groups. In some embodiments, Cy^{C} is imidazopyrimidinone, wherein Cy^{C} is substituted with 0-4 -L^{C}-R^{C} groups. In some embodiments, Cy^{C} is imidazopyrazinyl, wherein Cy^{C} is substituted with 0-4 -L^{C}-R^{C} groups. In some embodiments, Cy^{C} is benzoimidazolyl, wherein Cy^{C} is substituted with 0-4 -L^{C}-R^{C} groups. In some embodiments, Cy^{C} is triazolopyrimidinyl, wherein Cy^{C} is substituted with 0-3 -L^{C}-R^{C} groups. In some embodiments, Cy^{C} is thienopyridinyl, wherein Cy^{C} is substituted with 0-6-L^{C}-R^{C} groups. In some embodiments, Cy^{C} is quinolinyl, wherein Cy^{C} is substituted with 0-6-L^{C}-R^{C} groups.

In some embodiments, Cy^{C} is selected from the group consisting of:

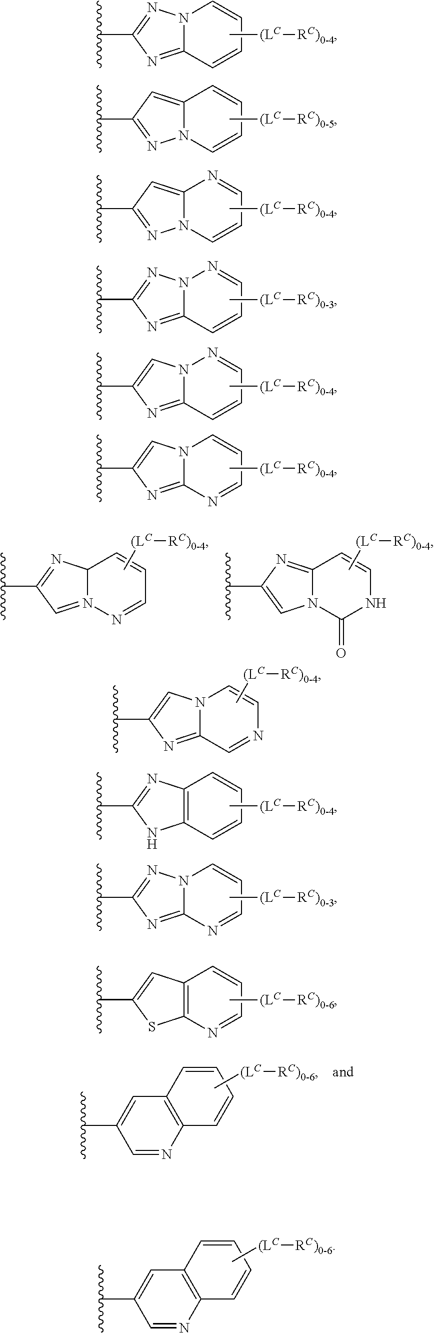

In some embodiments, Cy^{C} is selected from the group consisting of:

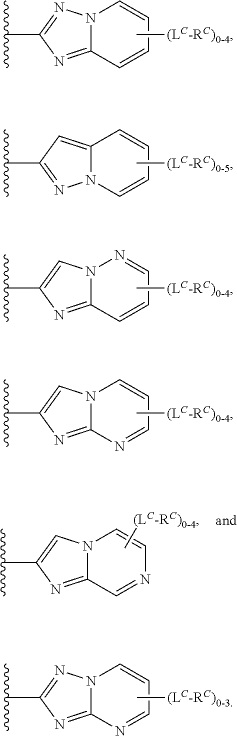

In some embodiments, Cy^{C} is selected from the group consisting of:

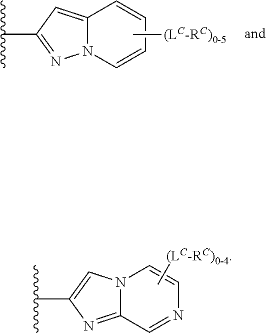

In some embodiments, a provided compound is of Formula II:

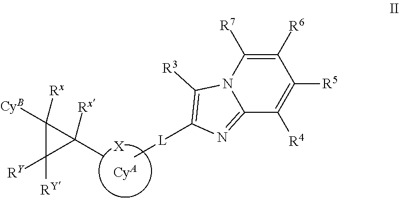

or a pharmaceutically acceptable salt thereof, wherein each of Cy^{A}, Cy^{B}, L, X, R^{x}, R^{x′}, R^{Y}, and R^{Y′} is defined and described in classes and subclasses herein, both singly and in combination, and each R³, R⁴, R⁵, R⁶, and R⁷ is independently selected from hydrogen or -L^{C}-R^{C}.

It will be understood that, unless otherwise specified or prohibited by the foregoing definition of Formula II embodiments of variables Cy^{A}, Cy^{B}, L, X, R^{x}, R^{x′}, R^{Y}, R^{Y′}, R³, R⁴, R⁵, R⁶, and R⁷ as defined above and described in classes and subclasses herein, also apply to compounds of Formula II both singly and in combination.

In some embodiments, each of R³, R⁴, R⁵, R⁶, and R⁷ is independently selected from hydrogen or -L^{C}-R^{C}, wherein each L^{C} is independently selected from a covalent bond or an optionally substituted C₁₋₆ hydrocarbon chain, wherein 1 to 3 methylene units are optionally and independently replaced with —O— or —NR—; and wherein each R^{C} is independently selected from halogen, —CN, —C(O)R, —C(O)₂R, —C(O)N(R)₂, —N(R)₂, —N(R)C(O)R, —N(R)C(O)₂R, —N(R)S(O)₂R, —S(O)₂R, —S(O)₂N(R)₂, Cy^{D}, or an optionally substituted group selected from C₁₋₆ aliphatic.

In some embodiments, R³ is selected from hydrogen or L^{C}-R^{C}, wherein L^{C} is a covalent bond and R^{C} is halogen. In some embodiments, R³ is hydrogen.

In some embodiments, R⁴ is selected from hydrogen or L^{C}-R^{C}, wherein L^{C} is selected from a covalent bond or an optionally substituted C₁₋₆ hydrocarbon chain, wherein 1 to 3 methylene units are optionally and independently replaced with —O— or —NR—; and wherein R^{C} is selected from halogen, —CN, —C(O)R, —C(O)₂R, —C(O)N(R)₂, —N(R)₂, —N(R)C(O)R, —N(R)C(O)₂R, —N(R)S(O)₂R, —OR, —S(O)₂R, —S(O)₂N(R)₂, Cy^{D}, or an optionally substituted group selected from C₁₋₆ aliphatic.

In some embodiments, R⁴ is selected from hydrogen or L^{C}-R^{C}, wherein L^{C} is a covalent bond and wherein R^{C} is selected from halogen, —CN, —C(O)R, —C(O)₂R, —C(O)N(R)₂, —N(R)₂, —N(R)C(O)R, —N(R)C(O)₂R, —N(R)S(O)₂R, —OR, —S(O)₂R, —S(O)₂N(R)₂, Cy^{D}, or an optionally substituted group selected from C₁₋₆ aliphatic.

In some embodiments, R⁴ is selected from the group consisting of:

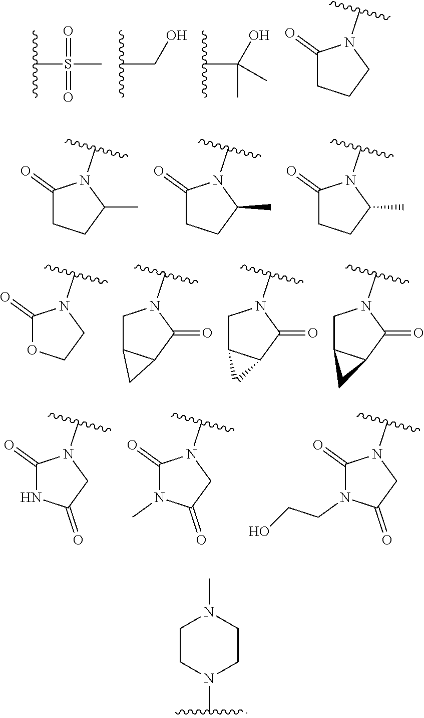

In some embodiments of R⁴, optional substituents on a C₁₋₆ aliphatic group are selected from —(CH₂)₀₋₄R^{∘}, —(CH₂)₀₋₄OR^{∘}, —CN, —(CH₂)₀₋₄N(R^{∘})₂, and —(CH₂)₀₋₄C(O)OR^{∘}, wherein each R^{∘} is independently as defined above and described in classes and subclasses herein.

In some embodiments of R⁴, Cy^{D} is selected from a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, a 6- to 12-membered saturated or partially unsaturated fused bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur, wherein Cy^{D} is substituted with 0-4 -L^{D}-R^{D} groups. In some embodiments of R⁴, Cy^{D} is a 5-membered saturated or partially unsaturated monocyclic heterocyclyl having 1-2 heteroatoms selected from oxygen, nitrogen, or sulfur.

In some embodiments of R⁴, Cy^{D} is selected from the group consisting of:

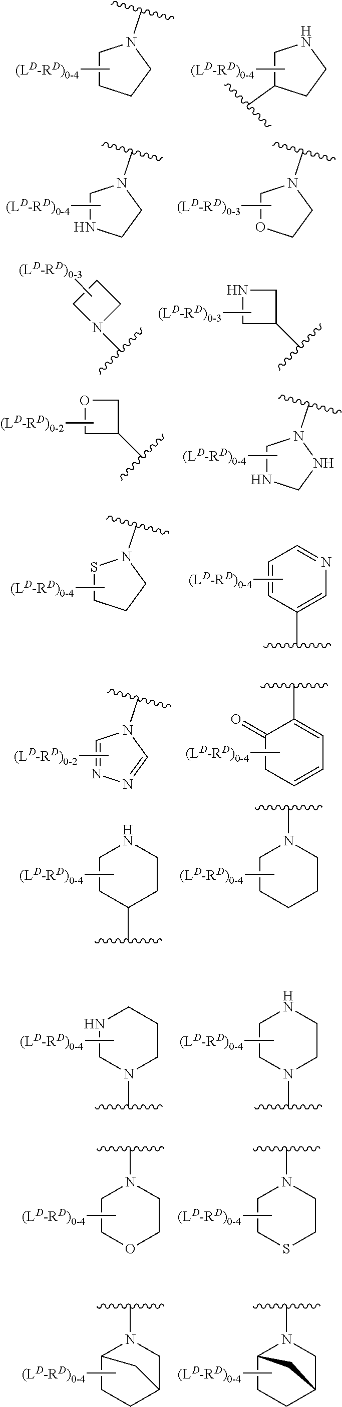

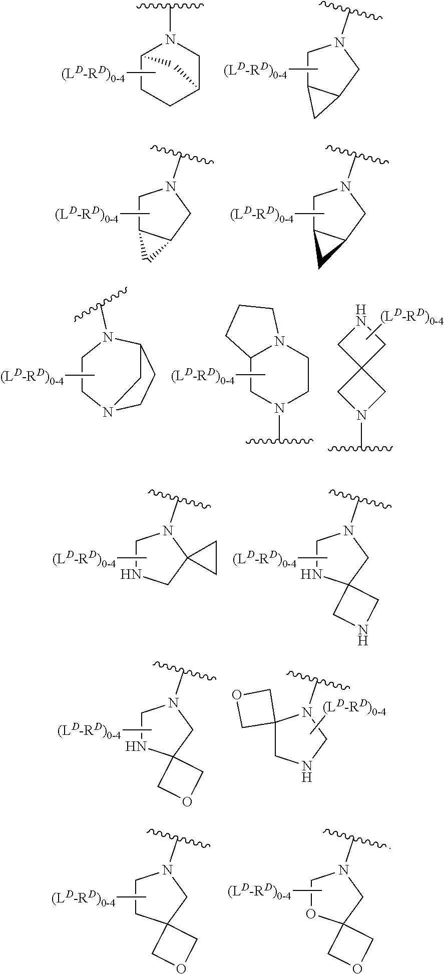

In some embodiments of R⁴, R^{D} is selected from oxo, halogen, —C(O)₂R, —N(R)₂, —OR, or an optionally substituted group selected from C₁₋₆ aliphatic, phenyl, a 3- to 7-membered saturated or partially unsaturated monocyclic carbocyclyl, or a 3- to 7-membered saturated or partially unsaturated monocyclic heterocyclyl having 1-2 heteroatoms selected from oxygen, nitrogen, or sulfur.

In some embodiments of a R^{D} group of R⁴, optional substituents on R^{D} are selected from halogen, —(CH₂)₀₋₄R^{∘}, —(CH₂)₀₋₄OR^{∘}, —(CH₂)₀₋₄N(R^{∘})₂, —(CH₂)₀₋₄C(O)OR^{∘}, and —OP(O)(OR^{∘})₂, wherein each R^{∘} is independently as defined above and described in classes and subclasses herein.

In some embodiments of R⁴, L^{D} is a covalent bond.

In some embodiments, R⁵ is hydrogen.

In some embodiments, R⁵ is L^{C}-R^{C}, wherein L^{C} is a covalent bond and R^{C} is Cy^{D}. In some embodiments, Cy^{D} is a cyclopropyl group.

In some embodiments, R⁶ is selected from hydrogen or L^{C}-R^{C}, wherein L^{C} is a covalent bond, and wherein R^{C} is selected from halogen, —N(R)₂, —OR, Cy^{D}, or an optionally substituted C₁₋₆-aliphatic group.

In some embodiments of R⁶, Cy^{D} is a cyclopropyl group substituted with 0-4 L^{D}-R^{D} groups. In some embodiments, L^{D} is a covalent bond and R^{D} is selected from halogen and optionally substituted C₁₋₆ aliphatic.

In some embodiments, R⁷ is selected from hydrogen or L^{C}-R^{C}, wherein L^{C} is a covalent bond, and wherein R^{C} is Cy^{D}.

In some embodiments, R⁷ is hydrogen.

In some embodiments of R⁷, Cy^{D} is:

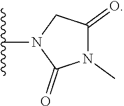 [SMILES: *N1CC(=O)N(C)C1=O]

In some embodiments, a provided compound is of Formula III:

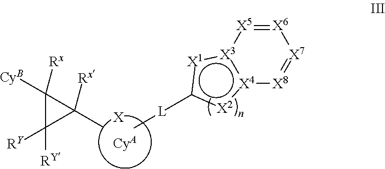

or a pharmaceutically acceptable salt thereof,
wherein each of Cy^{A}, Cy^{B}, L, X, R^{x}, R^{x′}, R^{Y}, and R^{Y′} is defined and described in classes and subclasses herein, both singly and in combination, and wherein
X¹ is N, CH, or C-L^{C}-R^{C};
each X² is independently selected from N, CH, or C-L^{C}-R^{C};
X³ and X⁴ is independently N or C, wherein at least one of X³ or X⁴ is C;
each of X⁵, X⁶, X⁷, and X⁸ are independently selected from N, CH, or C-L^{C}-R^{C}; and
n is 1 or 2.

In some embodiments, X¹ is N. In some embodiments, X¹ is CH. In some embodiments, X¹ is C-L^{C}-R^{C}, wherein L^{C} and R^{C} are as defined above and described in classes and subclasses herein, both singly and in combination.

In some embodiments, X³ is N or C and X⁴ is C. In some embodiments, X³ is C and X⁴ is N or C. In some embodiments, X³ is N and X⁴ is C. In some embodiments, X³ is C and X⁴ is C. In some embodiments, X³ is C and X⁴ is N.

In some embodiments, X⁵ is N. In some embodiments, X⁵ is CH. In some embodiments, X⁵ is C-L^{C}-R^{C}, wherein L^{C} and R^{C} are as defined above and described in classes and subclasses herein, both singly and in combination. In some embodiments, X⁶ is N. In some embodiments, X⁶ is CH. In some embodiments, X⁶ is C-L^{C}-R^{C}, wherein L^{C} and R^{C} are as defined above and described in classes and subclasses herein, both singly and in combination. In some embodiments, X⁷ is N. In some embodiments, X⁷ is CH. In some embodiments, X⁷ is C-L^{C}-R^{C}, wherein L^{C} and R^{C} are as defined above and described in classes and subclasses herein, both singly and in combination. In some embodiments, X⁸ is N. In some embodiments, X⁸ is CH. In some embodiments, X⁸ is C-L^{C}-R^{C}, wherein L^{C} and R^{C} are as defined above and described in classes and subclasses herein, both singly and in combination.

In some embodiments, n is 1. In some embodiments, n is 2.

In some embodiments, n is 1 and X⁸ is N. In some embodiments, n is 1 and X⁸ is CH. In some embodiments, n is 1 and X⁸ is C-L^{C}-R^{C}, wherein L^{C} and R^{C} are as defined above and described in classes and subclasses herein, both singly and in combination.

In some embodiments, n is 2 and each X² is independently selected from N, CH, or C-L^{C}-R^{C}, wherein L^{C} and R^{C} are as defined above and described in classes and subclasses herein, both singly and in combination. In some embodiments, n is 2 and one X² is N, and the other is CH. In some embodiments, n is 2 and both occurrences of X² are CH.

In some embodiments, a provided compound is of Formulae IV-a, IV-b, or IV-c:

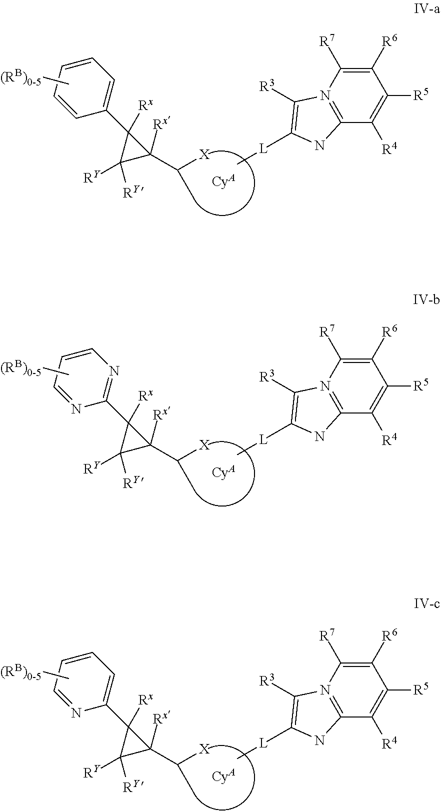

or a pharmaceutically acceptable salt thereof, wherein each of Cy^{A}, R^{B}, L, X, R^{x}, R^{x′}, R^{Y}, R^{Y′}, R³, R⁴, R⁵, R⁶, and R⁷ is defined and described in classes and subclasses herein, both singly and in combination.

It will be understood that, unless otherwise specified or prohibited by the foregoing definition of Formulae IV-a, IV-b, or IV-c embodiments of variables Cy^{A}, R^{B}, L, X, R^{x}, R^{x′}, R^{Y}, R^{Y′}, R³, R⁴, R⁵, R⁶, and R⁷ as defined above and described in classes and subclasses herein, also apply to compounds of Formulae IV-a, IV-b, or IV-c, both singly and in combination.

In some embodiments, a provided compound is of Formulae V-a, V-b, or V-c:

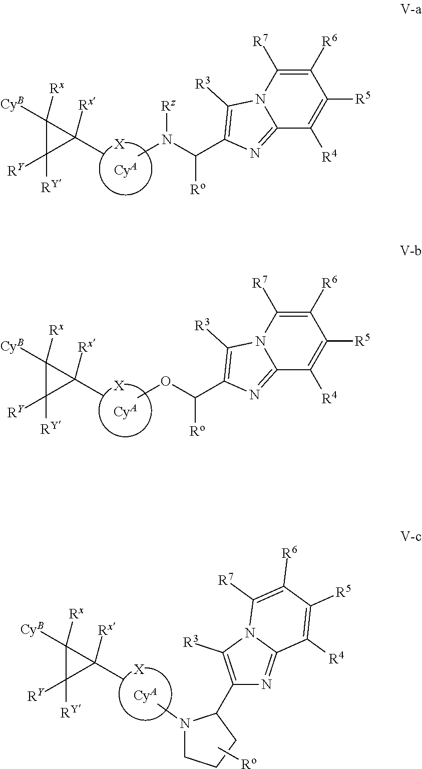

or a pharmaceutically acceptable salt thereof, wherein each of Cy^{A}, Cy^{B}, X, R^{∘}, R^{x}, R^{x′}, R^{Y}, R^{Y′}, R³, R⁴, R⁵, R⁶, and R⁷ is defined and described in classes and subclasses herein, both singly and in combination. In some embodiments of Formulae V-a and V-b, R^{∘} is hydrogen or methyl. In some embodiments of Formulae V-c, R^{∘} is hydrogen or —OH.

It will be understood that, unless otherwise specified or prohibited by the foregoing definition of Formulae V-a, V-b, or V-c embodiments of variables Cy^{A}, Cy^{B}, X, R^{∘}, R^{x}, R^{x′}, R^{Y}, R^{Y′}, R³, R⁴, R⁵, R⁶, and R⁷ as defined above and described in classes and subclasses herein, also apply to compounds of Formulae V-a, V-b, or V-c, both singly and in combination.

In some embodiments, a provided compound is of Formulae VI-a, VI-b, or VI-c:

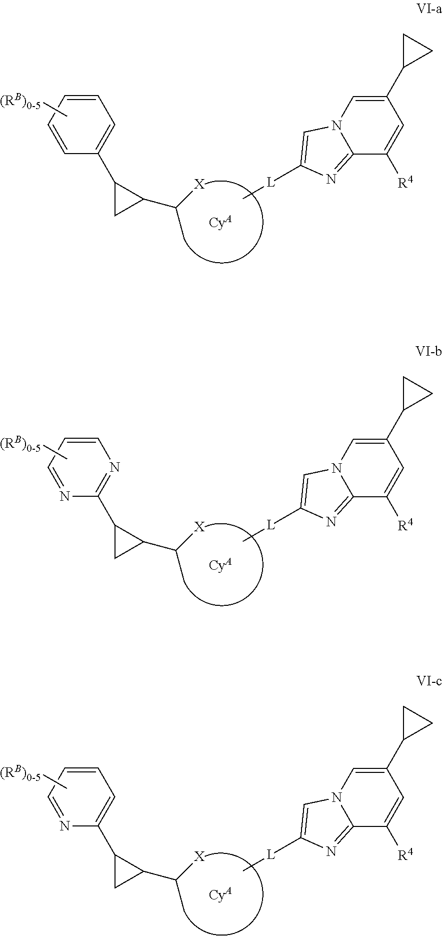

or a pharmaceutically acceptable salt thereof, wherein each of Cy^{A}, R^{B}, L and X is defined and described in classes and subclasses herein, both singly and in combination; and
R⁴ is hydrogen or L^{C}-R^{C}, wherein L^{C} is a covalent bond and R^{C} is halogen or Cy^{D}, wherein Cy^{D} is a 5-membered saturated or partially unsaturated monocyclic heterocyclyl having 1-2 heteroatoms selected from nitrogen, and wherein Cy^{D} is substituted with 0-4 -L^{D}-R^{D} groups.

It will be understood that, unless otherwise specified or prohibited by the foregoing definition of Formulae VI-a, VI-b, or VI-c embodiments of variables Cy^{A}, R^{B}, L, R⁴ and X as defined above and described in classes and subclasses herein, also apply to compounds of Formulae VI-a, VI-b, or VI-c, both singly and in combination.

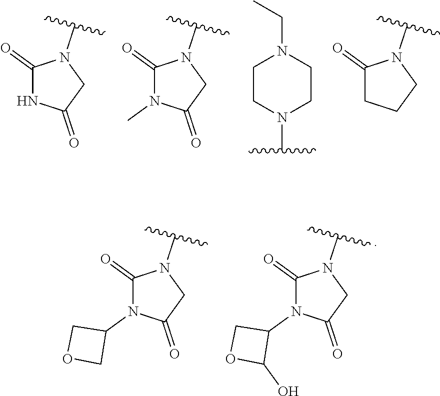

In some embodiments of Formulae VI-a, VI-b, or VI-c, Cy^{D} is a ring selected from: and ofany for Formula (I)-(VI-c), the moiety:

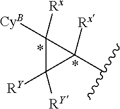

(including where one or more of R^{x}, R^{x′}, R^{Y}, or R^{Y′} is hydrogen) is in the relative trans configuration with respect to the Cy^{B} and Cy^{A} group attached to the two stereocenters marked with an *. In other words, it will be appreciated that “trans” in the context of the moiety:

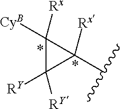

is meant a compound comprising a mixture of:

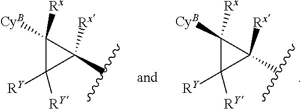

In some embodiments, such a mixture is a racemic mixture.

In certain embodiments of provided compounds (i.e., of any species not otherwise defined and of any of Formula (I)-(VI-c), the absolute stereochemistry of the moiety:

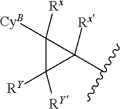

is as follows:

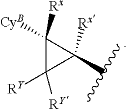

In certain embodiments of provided compounds (i.e., of any species not otherwise defined and of any of Formula (I)-(VI-c), the absolute stereochemistry of the moiety:

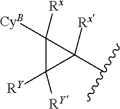

is as follows:

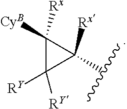

In some embodiments, a provided compound is selected Table A.

TABLE A
rac-N⁷-((6-cyclopropylimidazo[1,2-a]pyridin-2-yl)methyl)-N⁴,N⁴-dimethyl-2-((1S*,2S*)-2-(4-
methylpyrimidin-2-yl)cyclopropyl)quinoline-4,7-diamine (I-1);
2-((1S,2S)-2-(3-chlorophenyl)cyclopropyl)-7-(((6-cyclopropylimidazo[1,2-a]pyridin-2-
yl)methyl)amino)pyrido[4,3-d]pyrimidin-4(3H)-one (I-2);
rac-2-((1S*,2S*)-2-(3-chlorophenyl)cyclopropyl)-7-(((6-cyclopropylimidazo[1,2-a]pyridin-2-
yl)methyl)amino)pyrido[4,3-d]pyrimidin-4(3H)-one (1-3);
rac-1-(2-(((2-((1S*,2S*)-2-(3-chlorophenyl)cyclopropyl)-4-methoxyquinolin-7-yl)amino)methyl)-6-
cyclopropylimidazo[1,2-a]pyridin-8-yl)pyrrolidin-2-one (I-4);
rac-1-(2-(((2-((1S*,2S*)-2-(3-chlorophenyl)cyclopropyl)-4-oxo-1,4-dihydroquinolin-7-
yl)amino)methyl)-6-cyclopropylimidazo[1,2-a]pyridin-8-yl)-3-methylimidazolidine-2,4-dione (I-5);
1-(2-(((2-((1S,2S)-2-(3-chlorophenyl)cyclopropyl)-4-oxo-3,4-dihydroquinazolin-7-yl)amino)methyl)-6-
cyclopropylimidazo[1,2-a]pyridin-8-yl)-3-methylimidazolidine-2,4-dione (I-6);
rac-1-(6-cyclopropyl-2-(((2-((1S*,2S*)-2-(4-methylpyrimidin-2-yl)cyclopropyl)-4-oxo-3,4-
dihydropyrido[4,3-d]pyrimidin-7-yl)amino)methyl)imidazo[1,2-a]pyridin-8-yl)-3-methylimidazolidine-
2,4-dione (I-7);
rac-1-(6-cyclopropyl-2-(((2-((1S*,2S*)-2-(4-methylpyrimidin-2-yl)cyclopropyl)-4-oxo-1,4-dihydro
quinazolin-7-yl)amino)methyl)imidazo[1,2-a]pyridin-8-yl)-3-methylimidazolidine-2,4-dione (I-8);
rac-1-(6-cyclopropyl-2-(((2-((1S*,2S*)-2-(4-methylpyrimidin-2-yl)cyclopropyl)-4-oxo-3,4-dihydro
pyrido[3,2-d]pyrimidin-7-yl)amino)methyl)imidazo[1,2-a]pyridin-8-yl)-3-methylimidazolidine-2,4-
dione (I-9);
rac-1-(6-cyclopropyl-2-(((2-((1S*,2S*)-2-(4-methylpyrimidin-2-yl)cyclopropyl)-4-oxo-1,4-dihydro-
1,6-naphthyridin-7-yl)amino)methyl)imidazo[1,2-a]pyridin-8-yl)-3-methylimidazolidine-2,4-dione (I-
10)
rac-1-(6-cyclopropyl-2-(((2-((1S*,2S*)-2-(4-methylpyrimidin-2-yl)cyclopropyl)-4-oxo-1,4-dihydro
quinolin-7-yl)amino)methyl)imidazo[1,2-a]pyridin-8-yl)-3-methylimidazolidine-2,4-dione (I-11);
1-(6-cyclopropyl-2-(((2-((1S*,2S*)-2-(4-methylpyrimidin-2-yl)cyclopropyl)-4-oxo-1,4-
dihydroquinolin-7-yl)amino)methyl)imidazo[1,2-a]pyridin-8-yl)-3-methylimidazolidine-2,4-dione, first
eluting isomer (I-12);
1-(6-cyclopropyl-2-(((2-((1S*,2S*)-2-(4-methylpyrimidin-2-yl)cyclopropyl)-4-oxo-1,4-
dihydroquinolin-7-yl)amino)methyl)imidazo[1,2-a]pyridin-8-yl)-3-methylimidazolidine-2,4-dione,
second eluting isomer (I-13);
1-(6-cyclopropyl-2-(((2-((1S,2S)-2-(4-methylpyrimidin-2-yl)cyclopropyl)-4-oxo-1,4-dihydroquinolin-7-
yl)amino)methyl)imidazo[1,2-a]pyridin-8-yl)-3-methylimidazolidine-2,4-dione;
1-(6-cyclopropyl-2-(((2-((1R,2R)-2-(4-methylpyrimidin-2-yl)cyclopropyl)-4-oxo-1,4-dihydroquinolin-
7-yl)amino)methyl)imidazo[1,2-a]pyridin-8-yl)-3-methylimidazolidine-2,4-dione;
1-(6-cyclopropyl-2-(((2-((1S,2S)-2-(4-methylpyrimidin-2-yl)cyclopropyl)-4-oxo-1,4-dihydroquinolin-7-
yl)amino)methyl)imidazo[1,2-a]pyridin-8-yl)-3-methylimidazolidine-2,4-dione;
1-(6-cyclopropyl-2-(((2-((1R,2R)-2-(4-methylpyrimidin-2-yl)cyclopropyl)-4-oxo-1,4-dihydroquinolin-
7-yl)amino)methyl)imidazo[1,2-a]pyridin-8-yl)-3-methylimidazolidine-2,4-dione;
rac-1-(6-cyclopropyl-2-(((6-((1S*,2S*)-2-(4-methylpyrimidin-2-yl)cyclopropyl)-1,5-naphthyridin-3-
yl)amino)methyl)imidazo[1,2-a]pyridin-8-yl)-3-methylimidazolidine-2,4-dione (I-14);
rac-1-(6-cyclopropyl-2-(((2-((1S*,2S*)-2-(4-methylpyrimidin-2-yl)cyclopropyl)-1,6-naphthyridin-7-
yl)amino)methyl)imidazo[1,2-a]pyridin-8-yl)-3-methylimidazolidine-2,4-dione formic acid salt (I-15);
rac-1-(6-Cyclopropyl-2-(((6-((1S*,2S*)-2-(4-methylpyrimidin-2-yl)cyclopropyl)quinolin-3-
yl)amino)methyl)imidazo[1,2-a]pyridin-8-yl)-3-methylimidazolidine-2,4-dione (I-16);
rac-1-(6-Cyclopropyl-2-((methyl(6-((1S*,2S*)-2-(4-methylpyrimidin-2-yl)cyclopropyl)quinolin-3-
yl)amino)methyl)imidazo[1,2-a]pyridin-8-yl)-3-methylimidazolidine-2,4-dione (I-17);
1-(2-(((2-((1S,2S)-2-(3-chlorophenyl)cyclopropyl)-4-methoxy-1,6-naphthyridin-7-yl)amino)methyl)-6-
cyclopropylimidazo[1,2-a]pyridin-8-yl)-3-methylimidazolidine-2,4-dione (I-18);
rac-N-((6-cyclopropylimidazo[1,2-a]pyridin-2-yl)methyl)-2-((1S*,2S*)-2-(4-methylpyrimidin-2-
yl)cyclopropyl)quinolin-7-amine (I-19);
rac-N-((6-cyclopropylimidazo[1,2-a]pyridin-2-yl)methyl)-4-methoxy-2-((1S*,2S*)-2-(4-
methylpyrimidin-2-yl)cyclopropyl)quinolin-7-amine formic acid salt (I-20);
rac-1-(6-cyclopropyl-2-(((4-methoxy-2-((1S*,2S*)-2-(4-methylpyrimidin-2-yl)cyclopropyl)quinolin-7-
yl)amino)methyl)imidazo[1,2-a]pyridin-8-yl)-3-methylimidazolidine-2,4-dione (I-21);
rac-1-(6-cyclopropyl-2-(((4-methoxy-2-((1S*,2S*)-2-(4-methylpyrimidin-2-yl)cyclopropyl)quinolin-7-
yl)amino)methyl)imidazo[1,2-a]pyridin-8-yl)-3-methylimidazolidine-2,4-dione (I-22), first eluting
isomer;
rac-1-(6-cyclopropyl-2-(((4-methoxy-2-((1S*,2S*)-2-(4-methylpyrimidin-2-yl)cyclopropyl)quinolin-7-
yl)amino)methyl)imidazo[1,2-a]pyridin-8-yl)-3-methylimidazolidine-2,4-dione (I-23), second eluting
isomer;
1-(6-cyclopropyl-2-(((4-methoxy-2-((1S,2S)-2-(4-methylpyrimidin-2-yl)cyclopropyl)quinolin-7-
yl)amino)methyl)imidazo[1,2-a]pyridin-8-yl)-3-methylimidazolidine-2,4-dione;
1-(6-cyclopropyl-2-(((4-methoxy-2-((1R,2R)-2-(4-methylpyrimidin-2-yl)cyclopropyl)quinolin-7-
yl)amino)methyl)imidazo[1,2-a]pyridin-8-yl)-3-methylimidazolidine-2,4-dione;
rac-1-(6-cyclopropyl-2-(((3-((1S*,2S*)-2-(4-methylpyrimidin-2-yl)cyclopropyl)quinolin-6-
yl)amino)methyl)imidazo[1,2-a]pyridin-8-yl)-3-methylimidazolidine-2,4-dione (I-24);
rac-1-(2-(((4-(1H-imidazol-1-yl)-2-((1S*,2S*)-2-(4-methylpyrimidin-2-yl)cyclopropyl)quinolin-7-
yl)amino)methyl)-6-cyclopropylimidazo[1,2-a]pyridin-8-yl)-3-methylimidazolidine-2,4-dione (I-25);
rac-1-(6-cyclopropyl-2-(((2-((1S*,2S*)-2-(4-methylpyrimidin-2-yl)cyclopropyl)-4-(1H-1,2,4-triazol-1-
yl)quinolin-7-yl)amino)methyl)imidazo[1,2-a]pyridin-8-yl)-3-methylimidazolidine-2,4-dione (I-26);
1-(6-cyclopropyl-2-(((4-(1-methyl-1H-pyrazol-4-yl)-2-((1S,2S)-2-(4-methylpyrimidin-2-
yl)cyclopropyl)quinolin-7-yl)amino)methyl)imidazo[1,2-a]pyridin-8-yl)-3-methylimidazolidine-2,4-
dione (I-27);
1-(6-cyclopropyl-2-(((5-methoxy-2-((1S,2S)-2-(4-methylpyrimidin-2-yl)cyclopropyl)quinolin-7-
yl)amino)methyl)imidazo[1,2-a]pyridin-8-yl)-3-methylimidazolidine-2,4-dione (I-28);
rac-1-(6-cyclopropyl-2-(((4-(dimethylamino)-2-((1S*,2S*)-2-(4-methylpyrimidin-2-
yl)cyclopropyl)quinolin-7-yl)amino)methyl)imidazo[1,2-a]pyridin-8-yl)-3-methylimidazolidine-2,4-
dione (I-29);
1-(2-(((4-(azetidin-1-yl)-2-((1S,2S)-2-(4-methylpyrimidin-2-yl)cyclopropyl)quinolin-7-
yl)amino)methyl)-6-cyclopropylimidazo[1,2-a]pyridin-8-yl)-3-methylimidazolidine-2,4-dione, formic
acid salt (I-30);
1-(2-(((4-(azetidin-1-yl)-2-((1S,2S)-2-(4-methylpyrimidin-2-yl)cyclopropyl)quinolin-7-
yl)amino)methyl)-6-cyclopropylimidazo[1,2-a]pyridin-8-yl)-3-methylimidazolidine-2,4-dione;
1-(6-cyclopropyl-2-(((4-(3-hydroxyazetidin-1-yl)-2-((1S,2S)-2-(4-methylpyrimidin-2-
yl)cyclopropyl)quinolin-7-yl)amino)methyl)imidazo[1,2-a]pyridin-8-yl)-3-methylimidazolidine-2,4-
dione (I-31);
rac-1-(6-cyclopropyl-2-(((4-(methylamino)-2-((1S*,2S*)-2-(4-methylpyrimidin-2-
yl)cyclopropyl)quinolin-7-yl)amino)methyl)imidazo[1,2-a]pyridin-8-yl)-3-methylimidazolidine-2,4-
dione, formic acid salt (I-32);
rac-1-(6-cyclopropyl-2-(((2-((1S*,2S*)-2-(4-methylpyrimidin-2-yl)cyclopropyl)-4-(oxetan-3-
yloxy)quinolin-7-yl)amino)methyl)imidazo[1,2-a]pyridin-8-yl)-3-methylimidazolidine-2,4-dione (I-33);
rac-N-(7-(((6-cyclopropyl-8-(3-methyl-2,4-dioxoimidazolidin-1-yl)imidazo[1,2-a]pyridin-2-
yl)methyl)amino)-2-((1S*,2S*)-2-(4-methylpyrimidin-2-yl)cyclopropyl)quinolin-4-yl)-N-
methylmethanesulfonamide, formic acid salt (I-34);
rac-N-(7-(((6-cyclopropyl-8-(3-methyl-2,4-dioxoimidazolidin-1-yl)imidazo[1,2-a]pyridin-2-
yl)methyl)amino)-2-((1S*,2S*)-2-(4-methylpyrimidin-2-yl)cyclopropyl)quinolin-4-yl)-N-
methylmethanesulfonamide;
rac-1-(6-cyclopropyl-2-(((4-((1-methylpiperidin-4-yl)oxy)-2-((1S*,2S*)-2-(4-methylpyrimidin-2-
yl)cyclopropyl)quinolin-7-yl)amino)methyl)imidazo[1,2-a]pyridin-8-yl)-3-methylimidazolidine-2,4-
dione (I-35);
1-(6-cyclopropyl-2-(((2-((1S,2S)-2-(4-methylpyrimidin-2-yl)cyclopropyl)-4-(2-oxa-6-
azaspiro[3.3]heptan-6-yl)quinolin-7-yl)amino)methyl)imidazo[1,2-a]pyridin-8-yl)-3-
methylimidazolidine-2,4-dione, formic acid salt (I-36);
1-(6-cyclopropyl-2-(((2-((1S,2S)-2-(4-methylpyrimidin-2-yl)cyclopropyl)-4-(2-oxa-6-
azaspiro[3.3]heptan-6-yl)quinolin-7-yl)amino)methyl)imidazo[1,2-a]pyridin-8-yl)-3-
methylimidazolidine-2,4-dione;
1-(6-cyclopropyl-2-(((4-(1,1-dioxidothiomorpholino)-2-((1S,2S)-2-(4-methylpyrimidin-2-
yl)cyclopropyl)quinolin-7-yl)amino)methyl)imidazo[1,2-a]pyridin-8-yl)-3-methylimidazolidine-2,4-
dione, formic acid salt (1-37);
1-(6-cyclopropyl-2-(((4-(1,1-dioxidothiomorpholino)-2-((1S,2S)-2-(4-methylpyrimidin-2-yl)cyclo
propyl)quinolin-7-yl)amino)methyl)imidazo[1,2-a]pyridin-8-yl)-3-methylimidazolidine-2,4-dione;
1-(6-cyclopropyl-2-(((4-((2-hydroxy-2-methylpropyl)amino)-2-((1S,2S)-2-(4-methylpyrimidin-2-yl)
cyclopropyl)quinolin-7-yl)amino)methyl)imidazo[1,2-a]pyridin-8-yl)-3-methylimidazolidine-2,4-dione
(I-38);
1-(6-cyclopropyl-2-(((4-(4-methylpiperazin-1-yl)-2-((1S,2S)-2-(4-methylpyrimidin-2-
yl)cyclopropyl)quinolin-7-yl)amino)methyl)imidazo[1,2-a]pyridin-8-yl)-3-methylimidazolidine-2,4-
dione, formic acid salt (I-39);
1-(6-cyclopropyl-2-(((4-(4-methylpiperazin-1-yl)-2-((1S,2S)-2-(4-methylpyrimidin-2-yl)cyclo
propyl)quinolin-7-yl)amino)methyl)imidazo[1,2-a]pyridin-8-yl)-3-methylimidazolidine-2,4-dione;
rac-1-(6-cyclopropyl-2-(((2-((1S*,2S*)-2-(4-methylpyrimidin-2-yl)cyclopropyl)-4-morpholinoquinolin-
7-yl)amino)methyl)imidazo[1,2-a]pyridin-8-yl)-3-methylimidazolidine-2,4-dione (I-40);
rac-1-(6-cyclopropyl-2-(((2-((1S*,2S*)-2-(4-methylpyrimidin-2-yl)cyclopropyl)-4-
morpholinoquinolin-7-yl)amino)methyl)imidazo[1,2-a]pyridin-8-yl)-3-methylimidazolidine-2,4-dione,
first eluting isomer (I-41);
rac-1-(6-cyclopropyl-2-(((2-((1S*,2S*)-2-(4-methylpyrimidin-2-yl)cyclopropyl)-4-morpholinoquinolin-
7-yl)amino)methyl)imidazo[1,2-a]pyridin-8-yl)-3-methylimidazolidine-2,4-dione, second eluting
isomer (I-42);
1-(6-cyclopropyl-2-(((2-((1S,2S)-2-(4-methylpyrimidin-2-yl)cyclopropyl)-4-morpholinoquinolin-7-
yl)amino)methyl)imidazo[1,2-a]pyridin-8-yl)-3-methylimidazolidine-2,4-dione;
1-(6-cyclopropyl-2-(((2-((1R,2R)-2-(4-methylpyrimidin-2-yl)cyclopropyl)-4-morpholinoquinolin-7-
yl)amino)methyl)imidazo[1,2-a]pyridin-8-yl)-3-methylimidazolidine-2,4-dione;
1-[6-cyclopropyl-2-[[2-[(1S,2S)-2-(4-methylpyrimidin-2-yl)cyclopropyl]-4-(2-oxa-6-
azaspiro[3.3]heptan-6-yl)-7-quinolyl]oxymethyl]imidazo[1,2-a]pyridin-8-yl]-3-methyl-imidazolidine-
2,4-dione (I-43);
8-((1S,2S)-2-(3-chlorophenyl)cyclopropyl)-7-((6-cyclopropylimidazo[1,2-a] pyridin-2-yl) methyl)-7H-
purine (I-44);
2-((1S,2S)-2-(3-chlorophenyl)cyclopropyl)-N-((6-cyclopropylimidazo[1,2-a]pyridin-2-yl)methyl)-1H-
benzo[d]imidazol-6-amine (I-45);
2-((1S,2S)-2-(3-chlorophenyl)cyclopropyl)-N-((6-cyclopropylimidazo[1,2-a]pyridin-2-yl)methyl)-1H-
benzo[d]imidazol-7-amine (I-46);
rac-tert-butyl (2-((1S*,2S*)-2-(3-chlorophenyl)cyclopropyl)-4-methoxyquinolin-7-yl)((6-
cyclopropylimidazo[1,2-a]pyridin-2-yl)methyl)carbamate (I-47);
rac-2-((1S*,2S*)-2-(3-chlorophenyl)cyclopropyl)-N-((6-cyclopropylimidazo[1,2-a]pyridin-2-
yl)methyl)-4-methoxyquinolin-7-amine (I-48);
rac-2-((1S*,2S*)-2-(3-chlorophenyl)cyclopropyl)-7-(((6-cyclopropylimidazo[1,2-a]pyridin-2-
yl)methyl)amino)quinolin-4(1H)-one (I-49);
2-((1S,2S)-2-(3-chlorophenyl)cyclopropyl)-7-(((6-cyclopropylimidazo[1,2-a]pyridin-2-
yl)methyl)amino)quinazolin-4(1H)-one (I-50);
2-((1S,2S)-2-(3-chlorophenyl)cyclopropyl)-7-(((6-cyclopropylimidazo[1,2-a]pyridin-2-
yl)methyl)amino)quinolin-4-ol (I-51);
7-((2R,4S)-4-((tert-butyldimethylsilyl)oxy)-2-(6-cyclopropylimidazo[1,2-a]pyridin-2-yl)pyrrolidin-1-
yl)-4-methoxy-2-((1RS,2RS)-2-(4-methylpyrimidin-2-yl)cyclopropyl)quinoline (I-52);
(3S,5R)-5-(6-cyclopropylimidazo[1,2-a]pyridin-2-yl)-1-(4-methoxy-2-(rac-(1S*,2S*)-2-(4-
methylpyrimidin-2-yl)cyclopropyl)quinolin-7-yl)pyrrolidin-3-ol (I-53);
rac-7-((2R,4S)-2-(6-cyclopropylimidazo[1,2-a]pyridin-2-yl)-4-hydroxypyrrolidin-1-yl)-2-((1S*,2S*)-2-
(4-methylpyrimidin-2-yl)cyclopropyl)quinolin-4-ol (I-54);
rac-6-(((6-cyclopropylimidazo[1,2-a]pyridin-2-yl)methyl)amino)-3-((1S*,2S*)-2-(4-methylpyrimidin-
2-yl)cyclopropyl)-4H-benzo[b][1,4]thiazine 1,1-dioxide (I-55);
rac-(3S,5R)-1-(2-((1S*,2S*)-2-(3-chlorophenyl)cyclopropyl)-3-fluoro-1H-pyrrolo[3,2-c]pyridin-6-yl)-
5-(6-cyclopropylimidazo[1,2-a]pyridin-2-yl)pyrrolidin-3-ol (I-56);
rac-1-(6-cyclopropyl-2-(((2-((1S*,2S*)-2-(4-methylpyrimidin-2-yl)cyclopropyl)quinolin-7-
yl)amino)methyl)imidazo[1,2-a]pyridin-8-yl)-3-methylimidazolidine-2,4-dione (I-57);
1-(6-cyclopropyl-2-(((2-((1R,2R)-2-(4-methylpyrimidin-2-yl)cyclopropyl)quinolin-7-yl)amino)methyl)
imidazo[1,2-a]pyridin-8-yl)-3-methylimidazolidine-2,4-dione, first eluting isomer (I-58);
1-(6-cyclopropyl-2-(((2-((1S,2S)-2-(4-methylpyrimidin-2-yl)cyclopropyl)quinolin-7-yl)amino)
methyl)imidazo[1,2-a]pyridin-8-yl)-3-methylimidazolidine-2,4-dione, second eluting isomer (I-59);
rac-1-(6-cyclopropyl-2-(((2-((1S*,2S*)-2-(4-methylpyrimidin-2-yl)cyclopropyl)quinazolin-7-yl)
amino)methyl)imidazo[1,2-a]pyridin-8-yl)-3-methylimidazolidine-2,4-dione (I-60);
rac-1-(6-cyclopropyl-2-(((3-((1S*,2S*)-2-(4-methylpyrimidin-2-yl)cyclopropyl)-1,1-dioxido-4H-benzo
[b][1,4]thiazin-6-yl)amino)methyl)imidazo[1,2-a]pyridin-8-yl)-3-methylimidazolidine-2,4-dione (I-61);
rac-1-(6-cyclopropyl-2-(((3-((1S*,2S*)-2-(4-methylpyrimidin-2-yl)cyclopropyl)quinoxalin-6-
yl)amino)methyl)imidazo[1,2-a]pyridin-8-yl)-3-methylimidazolidine-2,4-dione (I-62);
rac-1-(6-cyclopropyl-2-(((2-((1S*,2S*)-2-(4-methylpyrimidin-2-yl)cyclopropyl) pyrido[4,3-
d]pyrimidin-7-yl)amino)methyl)imidazo[1,2-a]pyridin-8-yl)-3-methylimidazolidine-2,4-dione (I-63);
rac-6-(((6-cyclopropylimidazo[1,2-a]pyridin-2-yl)methyl)amino)-3-((1S*,2S*)-2-(4-methylpyrimidin-
2-yl)cyclopropyl)-4H-benzo[e][1,2,4]thiadiazine 1,1-dioxide (I-64);
rac-6-(((6-cyclopropylimidazo[1,2-a]pyridin-2-yl)methyl)amino)-3-((1S*,2S*)-2-(4-methylpyrimidin-
2-yl)cyclopropyl)-4H-benzo[e][1,2,4]thiadiazine 1,1-dioxide, first eluting isomer (I-65);
rac-6-(((6-cyclopropylimidazo[1,2-a]pyridin-2-yl)methyl)amino)-3-((1S*,2S*)-2-(4-methylpyrimidin-
2-yl)cyclopropyl)-4H-benzo[e][1,2,4]thiadiazine 1,1-dioxide, second eluting isomer (I-66);
6-(((6-cyclopropylimidazo[1,2-a]pyridin-2-yl)methyl)amino)-3-((1S,2S)-2-(4-methylpyrimidin-2-
yl)cyclopropyl)-4H-benzo[e][1,2,4]thiadiazine 1,1-dioxide;
6-(((6-cyclopropylimidazo[1,2-a]pyridin-2-yl)methyl)amino)-3-((1R,2R)-2-(4-methylpyrimidin-2-
yl)cyclopropyl)-4H-benzo[e][1,2,4]thiadiazine 1,1-dioxide;
rac-1-(6-cyclopropyl-2-((2R,4S)-4-hydroxy-1-(4-methoxy-2-((1S*,2S*)-2-(4-methylpyrimidin-2-yl)
cyclopropyl)quinolin-7-yl)pyrrolidin-2-yl)imidazo[1,2-a]pyridin-8-yl)-3-methylimidazolidine-2,4-dione
(I-67);
rac-1-(6-cyclopropyl-2-((2R,4S)-4-hydroxy-1-(2-((1S*,2S*)-2-(4-methylpyrimidin-2-yl)cyclopropyl)
quinolin-7-yl)pyrrolidin-2-yl)imidazo[1,2-a]pyridin-8-yl)-3-methylimidazolidine-2,4-dione (I-68);
rac-1-(6-cyclopropyl-2-(((4-(2-hydroxyethoxy)-2-((1S*,2S*)-2-(4-methylpyrimidin-2-yl)cyclopropyl)
quinolin-7-yl)amino)methyl)imidazo[1,2-a]pyridin-8-yl)-3-methylimidazolidine-2,4-dione (I-69);
rac-1-(6-cyclopropyl-2-(((2-((1S*,2S*)-2-(4-methylpyrimidin-2-yl)cyclopropyl) quinolin-8-
yl)amino)methyl)imidazo[1,2-a]pyridin-8-yl)-3-methylimidazolidine-2,4-dione (I-70);
rac-1-(6-cyclopropyl-2-((5-((1S*,2S*)-2-(4-methylpyrimidin-2-yl)cyclopropyl)-3H-imidazo[4,5-
b]pyridin-3-yl)methyl)imidazo[1,2-a]pyridin-8-yl)-3-methylimidazolidine-2,4-dione (I-71);
rac-1-(6-cyclopropyl-2-((5-((1S*,2S*)-2-(4-methylpyrimidin-2-yl)cyclopropyl)-1H-imidazo[4,5-
b]pyridin-1-yl)methyl)imidazo[1,2-a]pyridin-8-yl)-3-methylimidazolidine-2,4-dione (I-72);
1-(6-cyclopropyl-2-((5-((1S,2S)-2-(4-methylpyrimidin-2-yl)cyclopropyl)-1H-imidazo[4,5-b]pyridin-1-
yl)methyl)imidazo[1,2-a]pyridin-8-yl)-3-methylimidazolidine-2,4-dione;
1-(6-cyclopropyl-2-((5-((1R,2R)-2-(4-methylpyrimidin-2-yl)cyclopropyl)-1H-imidazo[4,5-b]pyridin-1-
yl)methyl)imidazo[1,2-a]pyridin-8-yl)-3-methylimidazolidine-2,4-dione;
rac-7-(((6-cyclopropyl-8-(3-methyl-2,4-dioxoimidazolidin-1-yl)imidazo[1,2-a]pyridin-2-
yl)methyl)amino)-2-((1S*,2S*)-2-(4-methylpyrimidin-2-yl)cyclopropyl)quinoline-4-carbonitrile (I-73);
rac-N-((6-cyclopropyl-8-(4-ethylpiperazin-1-yl)imidazo[1,2-a]pyridin-2-yl) methyl)-2-((1S*,2S*)-2-(4-
methylpyrimidin-2-yl)cyclopropyl)quinolin-7-amine (I-74);
rac-1-(6-cyclopropyl-2-(((5-(methoxymethyl)-2-((1S*,2S*)-2-(4-methylpyrimidin-2-yl)cyclopropyl)
quinolin-7-yl)amino)methyl)imidazo[1,2-a]pyridin-8-yl)-3-methylimidazolidine-2,4-dione (I-75);
rac-1-(6-cyclopropyl-2-(((4-(methoxymethyl)-2-((1S*,2S*)-2-(4-methylpyrimidin-2-yl)cyclopropyl)
quinolin-7-yl)amino)methyl)imidazo[1,2-a]pyridin-8-yl)-3-methylimidazolidine-2,4-dione (I-76);
rac-1-(6-cyclopropyl-2-(((4-(hydroxymethyl)-2-((1S*,2S*)-2-(4-methylpyrimidin-2-yl)cyclopropyl)
quinolin-7-yl)amino)methyl)imidazo[1,2-a]pyridin-8-yl)-3-methylimidazolidine-2,4-dione (I-77);
rac-1-(6-cyclopropyl-2-(((2-((1S*,2S*)-2-(4-methylpyrimidin-2-yl)cyclopropyl)quinolin-7-
yl)amino)methyl)imidazo[1,2-a]pyridin-8-yl)-3-(oxetan-3-yl)imidazolidine-2,4-dione (I-78);
rac-1-(6-cyclopropyl-2-(((2-((1S*,2S*)-2-(4-methylpyrimidin-2-yl)cyclopropyl)-4,4-dioxido-1H-
pyrido[3,4-b][1,4]thiazin-7-yl)amino)methyl)imidazo[1,2-a]pyridin-8-yl)-3-methylimidazolidine-2,4-
dione (I-79);
rac-6-(((6-cyclopropylimidazo[1,2-a]pyridin-2-yl)methyl)(methyl)amino)-3-((1S*,2S*)-2-(4-
methylpyrimidin-2-yl)cyclopropyl)-4H-benzo[e][1,2,4]thiadiazine 1,1-dioxide (I-80);
rac-1-(6-cyclopropyl-2-(((2-((1S*,2S*)-2-(4-methylpyrimidin-2-yl)cyclopropyl)quinolin-7-
yl)amino)methyl)imidazo[1,2-a]pyridin-8-yl)imidazolidine-2,4-dione (I-81);
rac-7-(((6-cyclopropyl-8-(3-methyl-2,4-dioxoimidazolidin-1-yl)imidazo[1,2-a]pyridin-2-
yl)methyl)amino)-2-((1S*,2S*)-2-(4-methylpyrimidin-2-yl)cyclopropyl)quinoline-5-carbonitrile (I-82);
rac-4-chloro-2-((1S*,2S*)-2-(7-(((6-cyclopropyl-8-(3-methyl-2,4-dioxoimidazolidin-1-yl)imidazo[1,2-
a]pyridin-2-yl)methyl)amino)quinolin-2-yl)cyclopropyl)benzonitrile (I-83);
rac-1-(6-cyclopropyl-2-(((6-methoxy-2-((1S*,2S*)-2-(4-methylpyrimidin-2-yl)cyclopropyl)quinolin-7-
yl)amino)methyl)imidazo[1,2-a]pyridin-8-yl)-3-methylimidazolidine-2,4-dione (I-84);
rac-2-(7-(((6-cyclopropyl-8-(3-methyl-2,4-dioxoimidazolidin-1-yl)imidazo[1,2-a]pyridin-2-yl)methyl)
amino)-2-((1S*,2S*)-2-(4-methylpyrimidin-2-yl)cyclopropyl)quinolin-4-yl)acetonitrile (I-85);
rac-1-(6-cyclopropyl-2-(((5-hydroxy-7-((1S*,2S*)-2-(4-methylpyrimidin-2-yl)cyclopropyl)pyrido[2,3-
d]pyrimidin-2-yl)amino)methyl)imidazo[1,2-a]pyridin-8-yl)-3-methylimidazolidine-2,4-dione (I-86);
6-((1-(6-cyclopropylimidazo[1,2-a]pyridin-2-yl)ethyl)amino)-3-((1S,2S)-2-(4-methylpyrimidin-2-
yl)cyclopropyl)-4H-benzo[e][1,2,4]thiadiazine 1,1-dioxide (I-87);
rac-1-(6-cyclopropyl-2-(((5-(dimethylamino)-2-((1S*,2S*)-2-(4-methylpyrimidin-2-yl)cyclopropyl)
quinolin-7-yl)amino)methyl)imidazo[1,2-a]pyridin-8-yl)-3-methylimidazolidine-2,4-dione (I-88);
rac-7-chloro-6-(((6-cyclopropylimidazo[1,2-a]pyridin-2-yl)methyl)amino)-3-((1S*,2S*)-2-(4-
methylpyrimidin-2-yl)cyclopropyl)-4H-benzo[e][1,2,4]thiadiazine 1,1-dioxide (I-89);
rac-1-(6-cyclopropyl-2-(((2-((1S*,2S*)-2-(4-methylpyrimidin-2-yl)cyclopropyl)quinolin-7-yl)amino)
methyl)imidazo[1,2-a]pyridin-8-yl)-5-(3-hydroxyoxetan-3-yl)-3-methylimidazolidine-2,4-dione (I-90);
6-((6-cyclopropylimidazo[1,2-a]pyridin-2-yl)methoxy)-3-((1S,2S)-2-(4-methylpyrimidin-2-
yl)cyclopropyl)-4H-benzo[e][1,2,4]thiadiazine 1,1-dioxide (I-91);
rac-6-((6-cyclopropylimidazo[1,2-a]pyridin-2-yl)methoxy)-3-((1S*,2S*)-2-(4-methylpyrimidin-2-
yl)cyclopropyl)-4H-benzo[e][1,2,4]thiadiazine 1,1-dioxide (I-92);
4-chloro-2-((1S,2S)-2-(7-(((6-cyclopropyl-8-(3-methyl-2,4-dioxoimidazolidin-1-yl)imidazo[1,2-
alpyridin-2-yl)methyl)amino)quinolin-2-yl)cyclopropyl)benzonitrile (I-93);
6-(((6-cyclopropylimidazo[1,2-a]pyridin-2-yl)methyl)amino)-4-methyl-3-((1S,2S)-2-(4-
methylpyrimidin-2-yl)cyclopropyl)-4H-benzo[e][1,2,4]thiadiazine 1,1-dioxide (I-94);
rac-1-(6-cyclopropyl-2-(((4-(2-hydroxypropan-2-yl)-2-((1S*,2S*)-2-(4-methylpyrimidin-2-
yl)cyclopropyl)quinolin-7-yl)amino)methyl)imidazo[1,2-a]pyridin-8-yl)-3-methylimidazolidine-2,4-
dione (I-95);
3-methyl-1-(6-methyl-2-(((2-((1S,2S)-2-(4-methylpyrimidin-2-yl)cyclopropyl)-4-(2-oxa-6-azaspiro
[3.3]heptan-6-yl)quinolin-7-yl)oxy)methyl)imidazo[1,2-a]pyridin-8-yl)imidazolidine-2,4-dione (I-96);
1-(6-cyclopropyl-2-(((4-((2-hydroxy-2-methylpropyl)amino)-2-((1S,2S)-2-(4-methylpyrimidin-2-
yl)cyclopropyl)quinolin-7-yl)oxy)methyl)imidazo[1,2-a]pyridin-8-yl)-3-methylimidazolidine-2,4-dione
(I-97);
1-(6-cyclopropyl-2-((methyl(2-((1S,2S)-2-(4-methylpyrimidin-2-yl)cyclopropyl)-4-(2-oxa-6-
azaspiro[3.3]heptan-6-yl)quinolin-7-yl)amino)methyl)imidazo[1,2-a]pyridin-8-yl)-3-
methylimidazolidine-2,4-dione (I-98);
1-(6-cyclopropyl-2-(1-((2-((1S,2S)-2-(4-methylpyrimidin-2-yl)cyclopropyl)-4-(2-oxa-6-azaspiro[3.3]
heptan-6-yl)quinolin-7-yl)amino)ethyl)imidazo[1,2-a]pyridin-8-yl)-3-methylimidazolidine-2,4-dione (I-
99);
1-(6-cyclopropyl-2-(((4-((2-hydroxy-2-methylpropyl)(methyl)amino)-2-((1S,2S)-2-(4-methylpyrimidin-
2-yl)cyclopropyl)quinolin-7-yl)amino)methyl)imidazo[1,2-a]pyridin-8-yl)-3-methylimidazolidine-2,4-
dione (I-100);
1-(6-cyclopropyl-2-((R)-1-((2-((1S,2S)-2-(4-methylpyrimidin-2-yl)cyclopropyl)-4-(2-oxa-6-
azaspiro[3.3]heptan-6-yl)quinolin-7-yl)amino)ethyl)imidazo[1,2-a]pyridin-8-yl)-3-methylimidazolidine-
2,4-dione (I-101);
rac-1-(6-cyclopropyl-2-(((5-methoxy-2-((1S*,2S*)-2-(4-methylpyrimidin-2-yl)cyclopropyl)quinolin-7-
yl)oxy)methyl)imidazo[1,2-a]pyridin-8-yl)-3-methylimidazolidine-2,4-dione (I-102);
1-(6-cyclopropyl-2-(((2-((1S,2S)-2-(4-methylpyrimidin-2-yl)cyclopropyl)-4-(2-oxa-6-
azaspiro[3.3]heptan-6-yl)-1,6-naphthyridin-7-yl)oxy)methyl)imidazo[1,2-a]pyridin-8-yl)-3-
methylimidazolidine-2,4-dione (I-103);
rac-1-(2-(((5-methoxy-2-((1S*,2S*)-2-(4-methylpyrimidin-2-yl)cyclopropyl)quinolin-7-
yl)amino)methyl)-6-methylimidazo[1,2-a]pyridin-8-yl)-3-methylimidazolidine-2,4-dione (I-104);
6-(((6-cyclopropyl-8-fluoroimidazo[1,2-a]pyridin-2-yl)methyl)amino)-3-((1S,2S)-2-(4-
methylpyrimidin-2-yl)cyclopropyl)-4H-benzo[e][1,2,4]thiadiazine 1,1-dioxide (I-105);
rac-1-(6-cyclopropyl-2-(((2-((1S*,2S*)-2-(4-methylpyrimidin-2-yl)cyclopropyl)quinolin-7-
yl)amino)methyl)imidazo[1,2-a]pyrazin-8-yl)-3-methylimidazolidine-2,4-dione (I-106);
1-(5-cyclopropyl-2-(((2-((1S,2S)-2-(4-methylpyrimidin-2-yl)cyclopropyl)-4-(2-oxa-6-
azaspiro[3.3]heptan-6-yl)quinolin-7-yl)amino)methyl)pyrazolo[1,5-a]pyridin-7-yl)-3-methylimi
dazolidine-2,4-dione (I-107);
1-(5-cyclopropyl-2-(((2-((1S,2S)-2-(4-methylpyrimidin-2-yl)cyclopropyl)-4-(2-oxa-6-
azaspiro[3.3]heptan-6-yl)quinolin-7-yl)oxy)methyl)pyrazolo[1,5-a]pyridin-7-yl)-3-
methylimidazolidine-2,4-dione (I-108);

or a pharmaceutically salt thereof.

Compounds explicitly disclosed herein may be claimed as an individual compound, including where there is no reference to stereochemistry.

### C. Pharmaceutical Compositions

In another aspect, the present invention provides pharmaceutical compositions comprising a compound according to the disclosure, such as a compound of Formulae (I)-(VI-c), or a compound of Formulae (I)-(VI-c) or a compound named in the examples in combination with a pharmaceutically acceptable excipient (e.g., carrier).

The pharmaceutical compositions include optical isomers, diastereomers, or pharmaceutically acceptable salts of the inhibitors disclosed herein. A compound of Formulae (I)-(VI-c) included in the pharmaceutical composition may be covalently attached to a carrier moiety, as described above. Alternatively, a compound of Formulae (I)-(VI-c) included in the pharmaceutical composition is not covalently linked to a carrier moiety.

A “pharmaceutically acceptable carrier,” as used herein refers to pharmaceutical excipients, for example, pharmaceutically, physiologically, acceptable organic or inorganic carrier substances suitable for enteral or parenteral application that do not deleteriously react with the active agent. Suitable pharmaceutically acceptable carriers include water, salt solutions (such as Ringer's solution), alcohols, oils, gelatins, and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention.

The compounds of the invention can be administered alone or can be coadministered to the subject. Coadministration is meant to include simultaneous or sequential administration of the compounds individually or in combination (more than one compound). The preparations can also be combined, when desired, with other active substances (e.g. to reduce metabolic degradation).

In some embodiments, a test agent as described herein can be incorporated into a pharmaceutical composition for administration by methods known to those skilled in the art and described herein for provided compounds.

### D. Formulations

Compounds of the present invention can be prepared and administered in a wide variety of oral, parenteral, and topical dosage forms. Thus, the compounds of the present invention can be administered by injection (e.g. intravenously, intramuscularly, intracutaneously, subcutaneously, intraduodenally, or intraperitoneally). In some embodiments compounds of the present disclosure are administered orally. Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable carrier or excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compounds of the invention can also be incorporated into liposomes or administered via transdermal pumps or patches. Pharmaceutical admixtures suitable for use in the present invention include those described, for example, in Pharmaceutical Sciences (17th Ed., Mack Pub. Co., Easton, PA) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions of the present invention may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

### E. Effective Dosages

Pharmaceutical compositions provided by the present invention include compositions wherein the active ingredient is contained in a therapeutically effective amount, i.e., in an amount effective to achieve its intended purpose. The actual amount effective for a particular application will depend, inter alia, on the condition being treated. For example, when administered in methods to treat HAE, such compositions will contain an amount of active ingredient effective to achieve the desired result (e.g. inhibiting PKa and/or decreasing the amount of bradykinin in a subject).

The dosage and frequency (single or multiple doses) of compound administered can vary depending upon a variety of factors, including route of administration; size, age, sex, health, body weight, body mass index, and diet of the recipient; nature and extent of symptoms of the disease being treated (e.g., the disease responsive to PKa inhibition); presence of other diseases or other health-related problems; kind of concurrent treatment; and complications from any disease or treatment regimen. Other therapeutic regimens or agents can be used in conjunction with the methods and compounds of the invention.

For any provided compound or test agent, the therapeutically effective amount can be initially determined from cell culture assays. Target concentrations will be those concentrations of active compound(s) that are capable of decreasing PKa enzymatic activity as measured, for example, using the methods described.

Therapeutically effective amounts for use in humans may be determined from animal models. For example, a dose for humans can be formulated to achieve a concentration that has been found to be effective in animals. The dosage in humans can be adjusted by monitoring PKa inhibition and adjusting the dosage upwards or downwards, as described above.

Dosages may be varied depending upon the requirements of the patient and the compound being employed. The dose administered to a patient, in the context of the present invention, should be sufficient to effect a beneficial therapeutic response in the patient over time. The size of the dose also will be determined by the existence, nature, and extent of any adverse side effects. In some embodiments a compound of the disclosure or a pharmaceutical composition comprising the same is provided as a unit dose.

In one aspect, compounds provided herein display one or more improved pharmacokinetic (PK) properties (e.g., Cₘₐₓ, tₘₐₓ, Cₘᵢₙ, t_{1/2}, AUC, CL, bioavailability, etc.) when compared to a reference compound. In some embodiments, a reference compound is a PKa inhibitor known in the art. In some embodiments, a reference compound is a PKa inhibitor selected from those disclosed in PCT Publication Number WO 2019/178129.

### F. Methods of Treatment

The present disclosure provides compounds and pharmaceutical compositions comprising the same for use in medicine, i.e. for use in treatment. The present disclosure further provides the use of any compounds described herein for inhibiting the activity of PKa, which would be beneficial to treatment of PKa-mediated diseases and conditions. Exemplary PKa-mediated disorders include edema, which refers to swelling in the whole body of a subject or a part thereof due to inflammation or injury when small blood vessels become leaky and releases fluid into nearby tissues. In some examples, the edema is HAE. In other examples, the edema occurs in eyes, e.g., diabetic macular edema (DME). The present disclosure provides methods of inhibiting the activity of PKa. In certain embodiments, the application provides a method of inhibiting the activity of PKa in vitro via contacting any of the compounds described herein with PKa molecules in a sample, such as a biological sample. In certain embodiments, the application provides a method of inhibiting the activity of PKa in vivo via delivering an effective amount of any of the compounds described herein to a subject in need of the treatment through a suitable route.

In certain embodiments, the methods comprise administering to a subject in need thereof (e.g., a subject such as a human patient, for example with edema) any of the compounds described herein or a pharmaceutically acceptable salt thereof. In certain embodiments, the methods comprise administering a compound of Formulae (I)-(VI-c), or a pharmaceutically acceptable salt or composition thereof, to a subject in need thereof. In some embodiments, the method comprises administering a pharmaceutical composition comprising a compound of Formulae (I)-(VI-c), or a pharmaceutically acceptable salt to a subject in need thereof.

In certain embodiments, the subject to be treated by any of the methods described herein is a human patient having, suspected of having, or at risk for edema, for example, HAE or diabetic macular edema (DME). A subject having an edema can be identified by routine medical examination, e.g., laboratory tests. A subject suspected of having an edema might show one or more symptoms of the disease/disorder. A subject at risk for edema can be a subject having one or more of the risk factors associated with the disease, for example, deficiency in C1-INH as for HAE.

In certain embodiments, provided herein are methods of alleviating one or more symptoms of HAE in a human patient who is suffering from an HAE attack. Such a patient can be identified by routine medical procedures. An effective amount of one or more of the provided compounds can be given to the human patient via a suitable route, for example, those described herein. The compounds described herein may be used alone, or may be used in combination with other anti-HAE agents, for example, a C1 esterase inhibitor (e.g., Cinryze® or Berinert®), a PKa inhibitor (e.g., ecallantide or lanadelumab) or a bradykinin B2 receptor antagonist (e.g., Firazyr®).

In other embodiments, provided herein are methods or reducing the risk of HAE attack in a human HAE patient who is in quiescent stage. Such a patient can be identified based on various factors, including history of HAE attack. An effective amount of one or more of the compounds can be given to the human patient via a suitable route, for example, those described herein. The compounds described herein may be used alone, or may be used in combination with other anti-HAE agents, for example, a C1 esterase inhibitor (e.g., Cinryze® or Berinert®), a PKa inhibitor (e.g., ecallantide or lanadelumab) or a bradykinin B2 receptor antagonist (e.g., Firazyr®).

In yet other embodiments, provided herein is prophylactic treatment of HAE in human patients having risk to HAE attacks with one or more of the compounds described herein. In some embodiments, patients suitable for prophylactic treatment of HAE are human subjects suffering from HAE (e.g., having history of HAE attacks). In some embodiments, patients suitable for such prophylactic treatment are human subjects where a physician determines a history of HAE attacks warrants a prophylactic approach (e.g., human subjects experiencing more than a particular average number of attacks over a time period, including by way of nonlimiting example, one, two, or more attacks per month). Alternatively, patients suitable for the prophylactic treatment may be human subjects having no HAE attack history but bearing one or more risk factors for HAE (e.g., family history, genetic defects in C1-INH gene, etc.) Such prophylactic treatment may involve the compounds described herein as the sole active agent, or involve additional anti-HAE agents, such as those described herein.

In certain embodiments, provided herein are methods for preventing or reducing edema in an eye of a subject (e.g., a human patient). In some examples, the human patient is a diabetic having, suspected of having, or at risk for diabetic macular edema (DME). DME is the proliferative form of diabetic retinopathy characterized by swelling of the retinal layers, neovascularization, vascular leak, and retinal thickening in diabetes mellitus due to leaking of fluid from blood vessels within the macula. To practice this method, an effective amount of one or more of the compounds described herein, or pharmaceutically acceptable salts thereof, may be delivered into the eye of the subject where treatment is needed. For example, the compound may be delivered topically, by intraocular injection, or intravitreal injection. A subject may be treated with the compound as described herein, either as the sole active agent, or in combination with another treatment for DME. Non-limiting examples of treatment for DME include laser photocoagulation, steroids, VEGF pathway targeting agents (e.g., Lucentis® (ranibizumab) or Eylea® (aflibercept)), and/or anti-PDGF agents.

In certain embodiments, the methods disclosed herein comprise administering to the subject an effective amount of a compound of Formulae (I)-(VI-c), or a pharmaceutically acceptable salt or composition thereof. In some embodiments, the effective amount is a therapeutically effective amount. In some embodiments, the effective amount is a prophylactically effective amount.

In certain embodiments, the subject being treated is an animal. The animal may be of either sex and may be at any stage of development. In certain embodiments, the subject is a mammal. In certain embodiments, the subject being treated is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent (e.g., mouse, rat), dog, pig, or non-human primate. In certain embodiments, the animal is a genetically engineered animal. In certain embodiments, the animal is a transgenic animal.

Certain methods described herein may comprise administering one or more additional pharmaceutical agent(s) in combination with the compounds described herein. The additional pharmaceutical agent(s) may be administered at the same time as the compound of Formulae (I)-(VI-c), or at different times than the compound of Formulae (I)-(VI-c). For example, the compound of Formulae (I)-(VI-c) and any additional pharmaceutical agent(s) may be on the same dosing schedule or different dosing schedules. All or some doses of the compound of Formulae (I)-(VI-c) may be administered before all or some doses of an additional pharmaceutical agent, after all or some does an additional pharmaceutical agent, within a dosing schedule of an additional pharmaceutical agent, or a combination thereof. The timing of administration of the compound of Formulae (I)-(VI-c) and additional pharmaceutical agents may be different for different additional pharmaceutical agents.

In certain embodiments, the additional pharmaceutical agent comprises an agent useful in the treatment of an edema, such as HAE or DME. Examples of such agents are provided herein.

Also provided is used of a compound of the present disclosure for the manufacture of a medicament for a condition/disease disclosed herein.

In the context of this specification “comprising” is to be interpreted as “including”. Embodiments of the invention comprising certain features/elements are also intended to extend to alternative embodiments “consisting” or “consisting essentially” of the relevant elements/features. Where technically appropriate, embodiments of the invention may be combined.

Technical references such as patents and applications are incorporated herein by reference.

Any embodiments specifically and explicitly recited herein may form the basis of a disclaimer either alone or in combination with one or more further embodiments.

The background section of this specification contains relevant technical information and may be used as basis for amendment. Subject headings herein are employed to divide the document into sections and are not intended to be used to construe the meaning of the disclosure provided herein.

The present specification claims priority from U.S. Provisional Application No. 63/162,483, filed Mar. 17, 2021, and incorporated herein by reference. This application may be used as basis for corrections to the present specification, especially in respect of chemical structures disclosed therein.

### IV. Examples

In certain embodiments, the Examples describe compounds comprising one or more stereocenters, where a particular stereocenter is designated “S*” or “R*.” In both cases, the depiction of the “*” generally indicates that the exact configuration is unknown (e.g., for a compound with a single stereocenter, the depiction R*- or S*- indicates that either the R- or S-isomer was isolated, but the configuration at the stereocenter of the particular isomer isolated was not determined).

It will be appreciated that compounds described within the Examples may comprise more than one stereocenter. As described above, single stereochemical isomers as well as enantiomeric, diastereomeric, and geometric (or conformational) mixtures of the present compounds are within the scope of the invention. Within a particular compound name, where more than one “S*” or “R*” appear within a single pair of parentheses (e.g., “(1S*,2S*)”), it is understood that the S* and/or R* configurations are relative to each other. For example, a compound denoted “(1S*,2S*)-” or “(1R*,2R*)-” would be understood to refer specifically to either the “(1S,2S)-” or “(1R,2R)-” isomer, but not the “(1S,2R)-” or “(1R,2S)-” isomers. Furthermore, a compound denoted “rac-(1S*,2S*)-” or “rac-(1R*,2R*)-” would be understood to include a racemic mixture of the “(1S,2S)-” and “(1R,2R)-” isomers. Similarly, a compound denoted “(1S*,2R*)-” or “(1R*,2S*)-” would be understood to refer specifically to either the “(1R,2S)-” or “(1S,2R)-” isomer, but not the “(1S,2S)-” or “(1R,2R)-” isomers. In addition, a compound denoted “rac-(1R*,2S*)-” or “rac-(1S*,2R*)-” would be understood to include a racemic mixture of the “(1R,2S)-” and “(1S,2R)-” isomers. In certain embodiments, the Examples include schemes that depict compounds with one or more stereocenters. In some embodiments, the symbol “&” followed by a number appears adjacent to a stereocenter. In such cases, it is understood to include a mixture of both configurations (e.g., R- and S-) at that position.

In some embodiments, the term “or” followed by a number appears adjacent to a stereocenter. In such cases, it is understood to denote either an “R-” or “S-” isomer, but the particular isomer was not determined.

In some embodiments, the numbering following the symbol “&” or term “or” refers to one stereocenter's relation to another stereocenter in that compound. For example, where two stereocenters in a compound are each denoted with the same number (e.g., two instances of “&1”), it is understood that the configurations are relative to each other (e.g., if the structure is drawn as (S,S) and both stereocenters are denoted “&1”, it is understood to include a mixture of the (S,S) and (R,R) isomers, but not the (S,R) or (R,S) isomers). However, where each stereocenter is denoted with a different number (e.g., one instance of “&1” and one instance of “&2”), it is understood that that the configurations may be independent to each other (e.g., if the structure is drawn (S,S) and one stereocenter is denoted “&1” and one is denoted “&2,” it is understood to include a mixture of the (S,S), (S,R), (R,S), and (R,R) isomers).

### Synthesis of Intermediates

### Synthesis of (1S,2S)-2-(4-methylpyrimidin-2-yl)cyclopropane-1-carboxamide

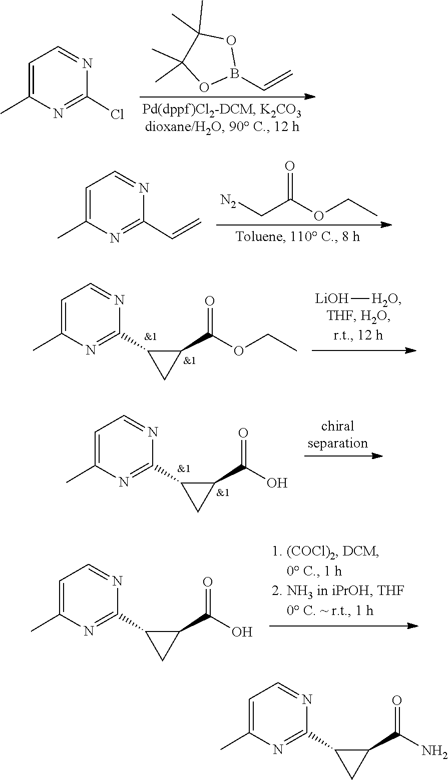

### Synthesis of 4-methyl-2-vinylpyrimidine

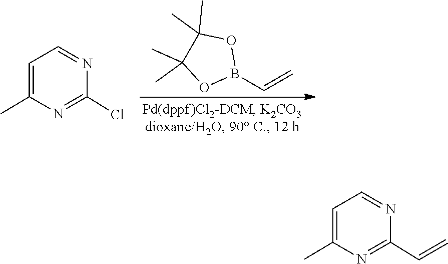

A mixture of 2-chloro-4-methylpyrimidine (6.4 g, 50 mmol), 4,4,5,5-tetramethyl-2-vinyl-1,3,2-dioxaborolane (10 g, 65 mmol), Pd(dppf)Cl₂-DCM (2 g, 2.5 mmol) and K₂CO₃ (17.25 g, 125 mmol) in dioxane (100 mL) and H₂O (5 mL) was stirred at 90° C. for 12 h. The reaction mixture was treated with H₂O (50 mL) and extracted with EA (50 mL×3). The combined organic layers were washed with brine (50 mL), dried over Na₂SO₄ and concentrated in vacuo (at 30° C.). The crude product was purified by silica column chromatography (PE/EtOAc=5/1) to give 4-methyl-2-vinylpyrimidine (4.8 g, 80%) as yellow oil. ESI-MS [M+H]⁺: 121.2.

### Synthesis of rac-ethyl (1S*, 2S*)-2-(4-methylpyrimidin-2-yl)cyclopropane-1-carboxylate

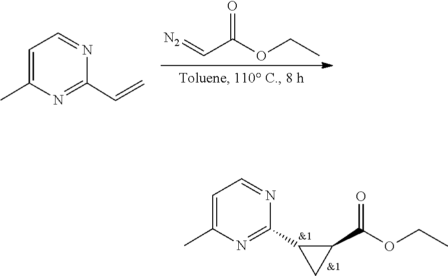

A solution of 4-methyl-2-vinylpyrimidine (4.8 g, 40 mmol) and ethyl 2-diazoacetate (9.12 g, 80 mmol) in toluene (70 mL) was refluxed at 110° C. for 8 h. The reaction was concentrated in vacuo and the crude product was purified by silica gel column (PE/EA=5/1) to give rac-ethyl (1S*, 2S*)-2-(4-methylpyrimidin-2-yl)cyclopropane-1-carboxylate (3.6 g, 43.7%) as yellow oil. ESI-MS [M+H]⁺: 207.2.

### Synthesis of rac-(1S*,2S*)-2-(4-methylpyrimidin-2-yl)cyclopropane-1-carboxylic acid

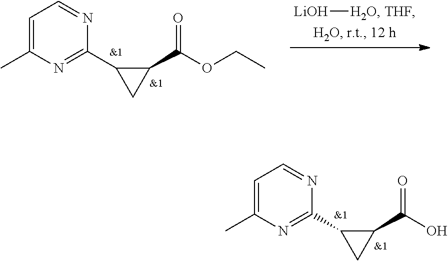

A mixture of rac-ethyl (1S*, 2S*)-2-(4-methylpyrimidin-2-yl)cyclopropane-1-carboxylate (3.6 g, 17.5 mmol) and LiOH—OH (1.4 g, 35 mmol) in THF/H₂O (20 mL/10 mL) was stirred at room temperature for 12 h. The reaction was concentrated in vacuo to remove THF and the pH of the residue was adjusted to 3 by HCl (2 N). The white solid was precipitated, and mixture was filtered, dried to give rac-(1S*,2S*)-2-(4-methylpyrimidin-2-yl)cyclopropane-1-carboxylic acid (2.4 g, 77%) as a white solid. ESI-MS [M+H]⁺: 179.2.

### Synthesis of (1S,2S)-2-(4-methylpyrimidin-2-yl)cyclopropane-1-carboxylic acid

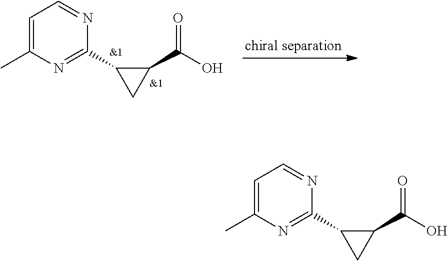

The racemic mixture form of rac-(1S*,2S*)-2-(4-methylpyrimidin-2-yl)cyclopropane-1-carboxylic acid (2.4 g) was chiral separated with SFC to give (1S,2S)-2-(4-methylpyrimidin-2-yl)cyclopropane-1-carboxylic acid (1.1 g, 45.8%) as a white solid. ESI-MS [M+H]+: 179.1.

### Synthesis of (1S,2S)-2-(4-methylpyrimidin-2-yl)cyclopropane-1-carboxamide

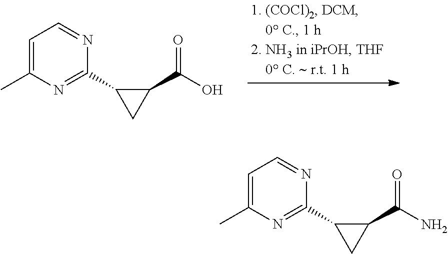

To a solution of (1S, 2S)-2-(4-methylpyrimidin-2-yl)cyclopropane-1-carboxylic acid (1.1 g, 6.1 mmol) in dry DCM (20 mL) was added (COCl)₂ (1.56 g, 12.3 mmol) at 0° C. slowly and was stirred at 0° C. for another 1 h. The reaction mixture was concentrated in vacuo and the resulting acid chloride was dissolved in dry THF (20 mL), cooled to 0° C. and then added NH₃ (20 mL, 2 M solution in iPrOH). The resulting solution was stirred at room temperature for another 1 h and concentrated in vacuo to give crude, which was purified with silica gel chromatography (eluent: DCM/MeOH=20/1) to furnish (1S,2S)-2-(4-methylpyrimidin-2-yl)cyclopropane-1-carboxamide (900 mg, 81.2%) as a yellow solid. ESI-MS [M+H]+: 178.1.

### Synthesis of (1S,2S)-2-(3-chlorophenyl)cyclopropane-1-carboxylic acid (first eluting isomer)

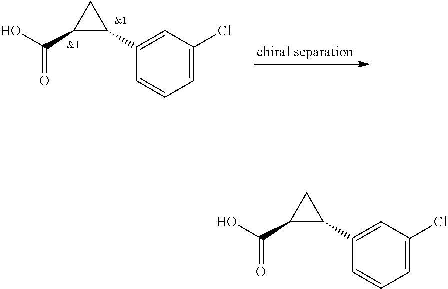 [SMILES: O=C(O)[C@H]1C[C@@H]1c1cccc(Cl)c1 ; O=C(O)[C@H]1C[C@@H]1c1cccc(Cl)c1]

rac-(1S*,2S*)-2-(3-chlorophenyl)cyclopropane-1-carboxylic acid (10 g) was separated using SFC (SFC80, Daicel CHIRALPAK AD-H 250 mm×20 mm I.D., 5 μm, CO₂/EtOH=86/14, 50 g/min, 35° C.) to give (1S,2S)-2-(3-chlorophenyl)cyclopropane-1-carboxylic acid (4.5 g, first eluting isomer, Rt=3.0 min, 99.9% e.e.) and (1R,2R)-2-(3-chlorophenyl)cyclopropane-1-carboxylic acid (4.3 g, second eluting isomer, Rt=4.0 min, 99.9% e.e.).

### Synthesis of 6-cyclopropylimidazo[1,2-a]pyridine-2-carbaldehyde

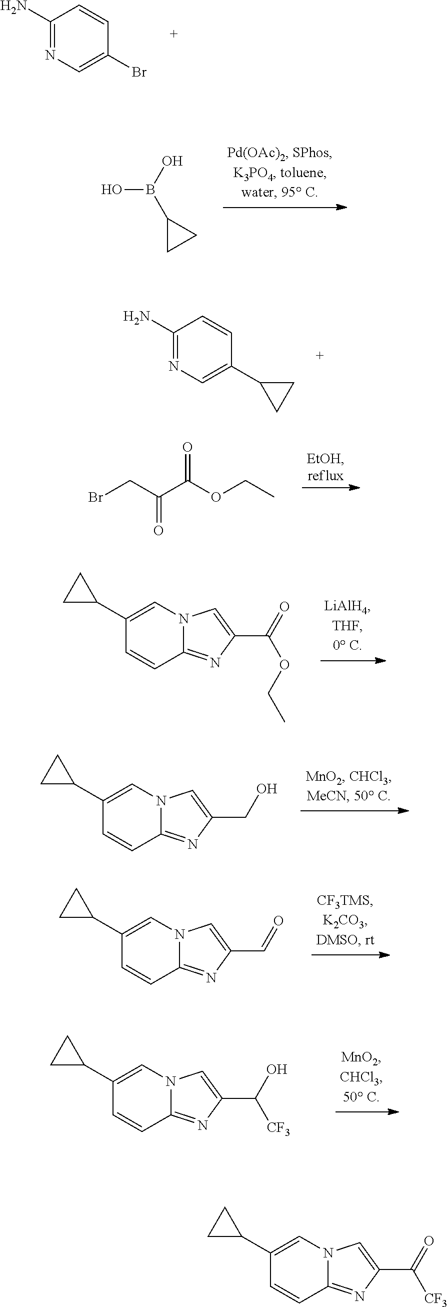

### Synthesis of 5-cyclopropylpyridin-2-amine

5-Bromopyridin-2-amine (10 g, 58 mmol), cyclopropylboronic acid (12 g, 120 mmol), SPhos (2.4 g, 5.8 mmol) and K₃PO₄ (43 g, 200 mmol) were placed under N₂ atmosphere and suspended in toluene (220 mL) and water (22 mL). The resulting suspension was degassed for 10 min and Pd(OAc)₂ (0.65 g, 2.9 mmol) was added. The reaction mixture was stirred at 95° C. for 16 h under N₂ atmosphere. The mixture was cooled to room temperature, diluted with water (30 mL) and extracted with ethyl acetate (3×30 mL). The organic phase was washed with brine (150 mL), dried over a hydrophobic frit and concentrated in vacuo. The residue was purified by silica gel chromatography eluting with a gradient of 10-95% EtOAc in hexane to give the title compound (5.3 g, 68%) as a red solid.

ESI-MS (M+H)+: 135.0, ¹H NMR (400 MHz, DMSO) δ 7.78 (d, J=2.5 Hz, 1H), 7.07 (dd, J=2.5, 8.6 Hz, 1H), 6.39 (d, J=8.4 Hz, 1H), 5.66 (s, 2H), 1.80-1.72 (m, 1H), 0.86-0.81 (m, 2H), 0.56-0.52 (m, 2H).

### Synthesis of ethyl 6-cyclopropylimidazo[1,2-a]pyridine-2-carboxylate

A solution of 5-cyclopropylpyridin-2-amine (5.3 g, 40 mmol), ethyl 3-bromo-2-oxopropanoate (5.9 mL, 47 mmol) in ethanol (110 mL) was stirred at reflux for 1 hour. The mixture was cooled to room temperature and concentrated in vacuo. The residue was dissolved in DCM (100 mL), washed with Na₂CO₃ (sat. aq., 60 mL), dried over a hydrophobic frit and concentrated in vacuo. The residue was purified by silica gel chromatography eluting with a gradient of 20-80% EtOAc in hexane to give the title compound (5 g, 54%) as an orange solid.

ESI-MS (M+H)+: 231.2, ¹H NMR (400 MHz, CDCl₃) δ 8.10-8.09 (m, 1H), 7.91-7.89 (m, 1H), 7.57 (d, J=9.5 Hz, 1H), 6.99 (dd, J=1.6, 9.5 Hz, 1H), 4.45 (q, J=7.1 Hz, 2H), 1.94-1.86 (m, 1H), 1.44 (t, J=7.2 Hz, 3H), 1.03-0.97 (m, 2H), 0.73-0.68 (m, 2H).

### Synthesis of (6-cyclopropylimidazo[1,2-a]pyridin-2-yl)methanol

LiAlH₄ (1M in THF, 42.99 mmol, 42.99 mL) was added to a solution of ethyl 6-cyclopropylimidazo[1,2-a]pyridine-2-carboxylate (21.50 mmol, 4.95 g) in THF (25 mL) cooled to 0° C., under N₂. The mixture was stirred for 1 hour then EtOAc (5 mL) was added dropwise. The mixture was stirred at room temperature for 1 hour then washed with water (30 mL) and brine (30 mL). The organics were dried over MgSO₄ then concentrated in vacuo to give the title compound which was used without further purification.

¹H NMR (400 MHz, CDCl₃) δ 7.86 (s, 1H), 7.46-7.42 (m, 2H), 6.92 (dd, J=1.8, 9.4 Hz, 1H), 4.82 (s, 2H), 3.88-3.84 (m, 1H), 1.87 (s, 1H), 0.99-0.93 (m, 2H), 0.69-0.64 (m, 2H).

### Synthesis of 6-cyclopropylimidazo[1,2-a]pyridine-2-carbaldehyde

A suspension of (6-cyclopropylimidazo[1,2-a]pyridin-2-yl)methanol (2.0 g, 11 mmol), manganese(IV) oxide (9.2 g, 110 mmol) in chloroform (25 mL) and MeCN (25 mL) was stirred at 50° C. for 1 hour. The mixture was cooled to room temperature, filtered through Celite® and concentrated in vacuo to give the title compound (1.3 g, 66%).

ESI-MS (M+H)+: 187.2, ¹H NMR (400 MHz, CDCl₃) δ 10.13 (s, 1H), 8.06 (s, 1H), 7.93 (s, 1H), 7.57 (d, J=9.6 Hz, 1H), 7.03 (dd, J=1.8, 9.3 Hz, 1H), 1.95-1.87 (m, 1H), 1.05-0.99 (m, 2H), 0.75-0.69 (m, 2H).

### Synthesis of 2-(chloromethyl)-6-cyclopropylimidazo[1,2-a]pyridine

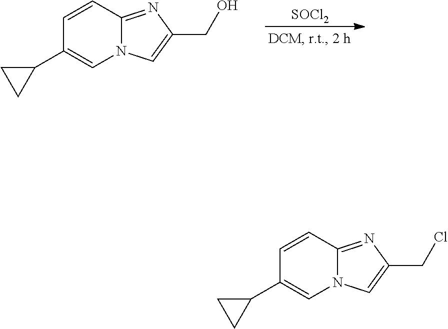 [SMILES: OCc1cn2cc(C3CC3)ccc2n1 ; "SOCl2" ; ClCc1cn2cc(C3CC3)ccc2n1]

To a solution of (6-cyclopropylimidazo[1,2-a]pyridin-2-yl)methanol (0.13 g, 0.69 mmol) in DCM (3.0 mL) was added SOCl₂ (1.0 mL) slowly at 0° C. The reaction mixture was stirred at room temperature for 2 h. The resulting mixture was concentrated in vacuo to give 2-(chloromethyl)-6-cyclopropylimidazo[1,2-a]pyridine (0.14 g, 98%) as a yellow solid which was used without further purification.

### Synthesis of 2-((8-bromo-6-cyclopropylimidazo[1,2-a]pyridin-2-yl)methyl)isoindoline-1,3-dione

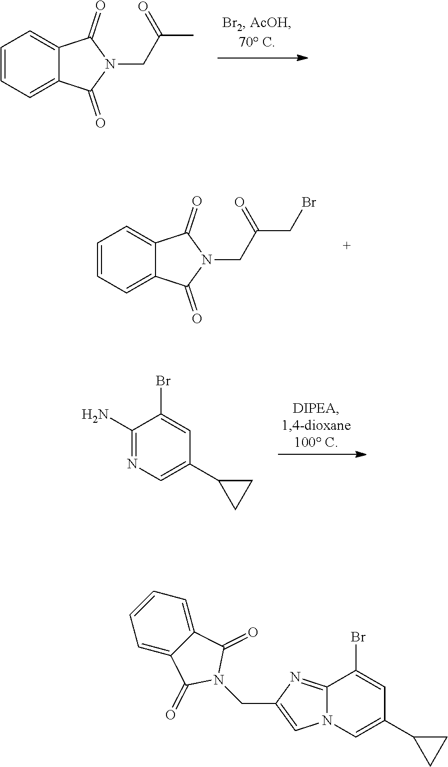 [SMILES: CC(=O)CN1C(=O)c2ccccc2C1=O ; O=C(CBr)CN1C(=O)c2ccccc2C1=O ; Nc1ncc(C2CC2)cc1Br ; O=C1c2ccccc2C(=O)N1Cc1cn2cc(C3CC3)cc(Br)c2n1]

### Synthesis of 2-(3-bromo-2-oxopropyl)isoindoline-1,3-dione

A few drops of a solution of bromine (7.6 mL, 150 mmol) in AcOH (80 mL) was added to a stirred solution of 2-(2-oxopropyl)isoindoline-1,3-dione (20 g, 98 mmol) at 70° C. The reaction was stirred until the solution went colourless. The remainder of the bromine/AcOH solution was added dropwise over 2 h. The reaction mixture was stirred at 70° C. for a further 2 h. The mixture was cooled to room temperature and concentrated in vacuo. The residue was dissolved in DCM (300 mL) and washed with Na₂S₂O₃ solution (1.0 M, 75 mL) and Na₂CO₃ solution (10% aq., 2×150 mL). The combined organics were dried over MgSO₄ and concentrated in vacuo. The residue was triturated with hot diethyl ether, filtered and dried to give the title compound (22.6 g, 81%) as a white solid.

¹H NMR (400 MHz, CDCl₃) δ 7.92-7.87 (m, 2H), 7.79-7.73 (m, 2H), 4.78 (s, 2H), 4.01 (s, 2H).

### Synthesis of 2-((8-bromo-6-cyclopropylimidazo[1,2-a]pyridin-2-yl)methyl)isoindoline-1,3-dione

A solution of 2-(3-bromo-2-oxopropyl)isoindoline-1,3-dione (12 g, 39 mmol), 3-bromo-5-cyclopropylpyridin-2-amine (7.5 g, 35 mmol) and DIPEA (9.2 mL, 53 mmol) in 1,4-dioxane (350 mL) was heated at 100° C. for 16 h. The mixture was cooled to room temperature and the volume was reduced by a half by concentrating in vacuo. The mixture was diluted with DCM (200 mL) and washed with NaHCO₃ solution (sat. aq., 150 mL) and brine (150 mL). The organics were dried over MgSO₄ and concentrated in vacuo. The residue was purified by column chromatography on silica gel, eluting with a gradient of 0-100% EtOAc in cyclohexane to give the title compound (7.2 g, 52%).

ESI-MS (M+H)+: 396.1, 398.1 ¹H NMR (400 MHz, DMSO) δ 8.35 (d, J=1.0 Hz, 1H), 7.99-7.94 (m, 2H), 7.94-7.91 (m, 3H), 7.39 (d, J=1.5 Hz, 1H), 4.94 (s, 2H), 2.02-1.94 (m, 1H), 0.96 (m, 2H), 0.76-0.71 (m, 2H).

### Synthesis of 2-((8-chloro-6-cyclopropylimidazo[1,2-a]pyridin-2-yl)methyl)isoindoline-1,3-dione

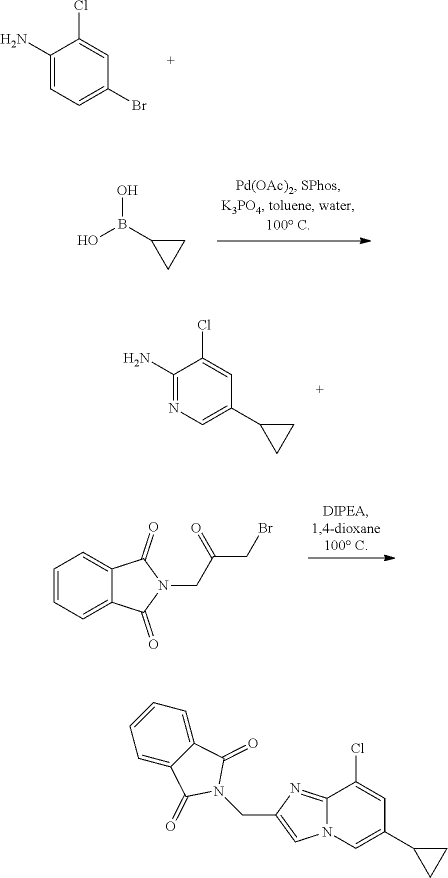 [SMILES: Nc1ccc(Br)cc1Cl ; OB(O)C1CC1 ; Nc1ncc(C2CC2)cc1Cl ; O=C(CBr)CN1C(=O)c2ccccc2C1=O ; O=C1c2ccccc2C(=O)N1Cc1cn2cc(C3CC3)cc(Cl)c2n1]

### Synthesis of 3-chloro-5-cyclopropylpyridin-2-amine

A mixture of toluene (120 mL) and water (12 mL) were degassed for 50 min. 5-Bromo-3-chloropyridin-2-amine (5.0 g, 24 mmol), cyclopropylboronic acid (2.1 g, 24 mmol), SPhos (0.99 g, 2.4 mmol) and K₃PO₄ (18 g, 84 mmol) were added to the solvent mixture, and it was heated to 100° C. Pd(OAc)₂ (0.27 g, 1.2 mmol) was added and the reaction mixture was stirred at this temperature for 3 h. The mixture was cooled to room temperature and filtered through Celite®, which was washed with toluene. The combined organics were dried over MgSO₄ and concentrated in vacuo. The residue was purified by column chromatography on silica gel, eluting with a gradient of 5-30% EtOAc in cyclohexane to give the title compound (3.5 g, 85%) as an orange solid.

ESI-MS (M+H)+: 169.0, ¹H NMR (400 MHz, CDCl₃) δ 7.45 (s, 1H), 6.99 (d, J=1.5 Hz, 1H), 5.10 (s, 2H), 1.88-1.80 (m, 1H), 0.98-0.86 (m, 2H), 0.66-0.61 (m, 2H).

### Synthesis of 2-((8-chloro-6-cyclopropylimidazo[1,2-a]pyridin-2-yl)methyl)isoindoline-1,3-dione

A solution of 2-(3-bromo-2-oxopropyl)isoindoline-1,3-dione (890 mg, 3.1 mmol), 3-chloro-5-cyclopropylpyridin-2-amine (530 mg, 3.1 mmol) in 1,4-dioxane (11 mL) was heated at 98° C. for 17 h. The mixture was cooled to room temperature and concentrated in vacuo. The mixture was partitioned between DCM (2×50 mL) and NaHCO₃ (sat. aq., 50 mL). The organics were dried over MgSO₄ and concentrated in vacuo. The residue was purified by column chromatography on silica gel, eluting with a gradient of 10-50% EtOAc in isohexane to give the title compound (880 mg, 84%).

ESI-MS (M+H)+: 352.1, ¹H NMR (400 MHz, CDCl₃) δ 7.91-7.85 (m, 2H), 7.76-7.70 (m, 3H), 7.46 (s, 1H), 6.99 (s, 1H), 5.10 (s, 2H), 1.87-1.80 (m, 1H), 0.99-0.89 (m, 2H), 0.67-0.60 (m, 2H).

### Synthesis of tert-butyl ((8-bromo-6-cyclopropylimidazo[1,2-a]pyridin-2-yl)methyl)carbamate

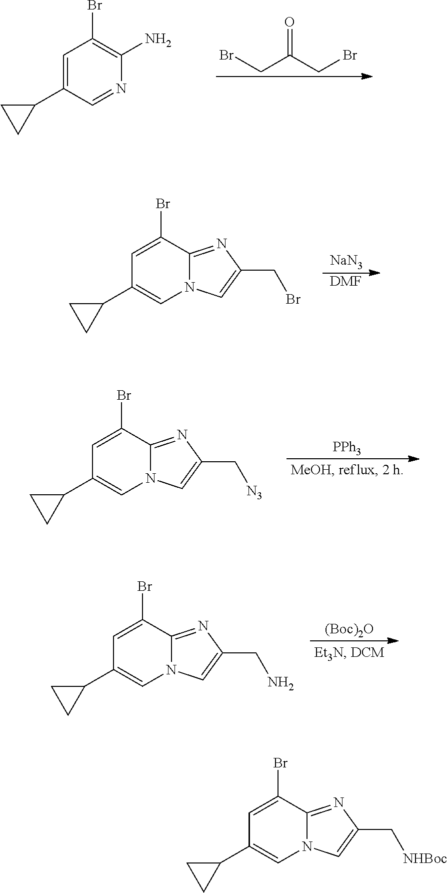

### Synthesis of 8-bromo-2-(Bromomethyl)-6-cycopropylimidazo[1,2-a]pyridine

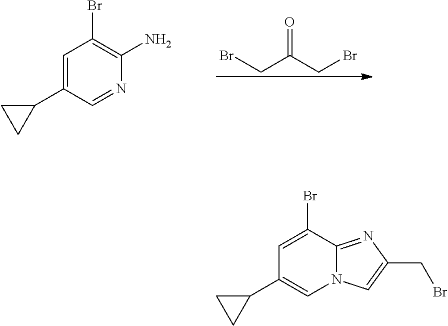 [SMILES: Nc1ncc(C2CC2)cc1Br ; O=C(CBr)CBr ; BrCc1cn2cc(C3CC3)cc(Br)c2n1]

A mixture of 3-bromo-5-cyclopropylpyridin-2-amine (1.06 g, 5.0 mmol) and 1,3-dibromopropan-2-one (1.62 g, 7.5 mmol) in DME (20.0 mL) was stirred at 90° C. under N₂ for 16 h. The reaction mixture was warmed to room temperature, quenched with sat. NaHCO₃ solution (50 mL) and extracted with EtOAc (3×50 ml). The combined organics were washed with brine (50 mL), dried over Na₂SO₄ and concentrated in vacuo. The crude residue was purified by column chromatography (eluent: DCM/MeOH=50/1˜30/1) to give the product 8-bromo-2-(bromomethyl)-6-cyclopropylimidazo[1,2-a]pyridine (1.2 g, 73%) as a yellow solid. ESI-MS [M+H]⁺: 330.2.

### Synthesis of 2-(azidomethyl)-8-bromo-6-cyclopropylimidazo[1,2-a]pyridine

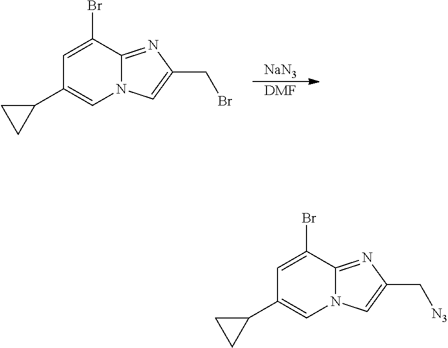

A mixture of 8-bromo-2-(bromomethyl)-6-cyclopropylimidazo[1,2-a]pyridine (1.0 g, 3.0 mmol) and NaN₃ (244 mg, 3.75 mmol) in DMF (10.0 mL) was stirred at room temperature under N₂ for 16 h. The mixture was diluted with EtOAc (100 mL) and washed with brine (3×50 mL). The organic layer was dried over Na₂SO₄ and concentrated in vacuo to give 2-(azidomethyl)-8-bromo-6-cyclopropylimidazo[1,2-a]pyridine (900 mg, crude) as a yellow solid which was used for the next step without purification. ESI-MS [M+H]⁺: 292.2.

### Synthesis of (8-bromo-6-cyclopropylimidazo[1,2-a]pyridin-2-yl)methanamine

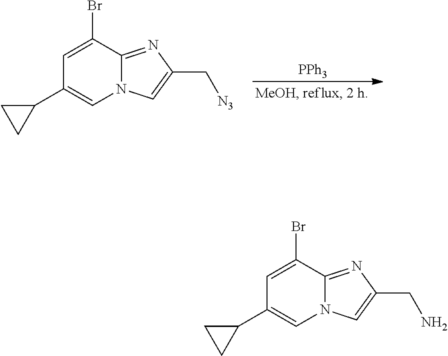 [SMILES: [N-]=[N+]=NCc1cn2cc(C3CC3)cc(Br)c2n1 ; NCc1cn2cc(C3CC3)cc(Br)c2n1 ; "PPh3"]

A mixture of 2-(azidomethyl)-8-bromo-6-cyclopropylimidazo[1,2-a]pyridine (870 mg, 3.0 mmol) and PPh₃ (983 mg, 3.75 mmol) in MeOH (25 mL) was stirred at reflux for 2 h. The mixture was concentrated in vacuo and purified by preparative TLC (eluent: DCM/MeOH=10/1) to give (8-bromo-6-cyclopropylimidazo[1,2-a]pyridin-2-yl)methanamine (550 mg, 69%) as a yellow oil. ESI-MS [M+H]⁺: 266.2.

### Synthesis of tert-butyl ((8-bromo-6-cyclopropylimidazo[1,2-a]pyridin-2-yl)methyl)carbamate

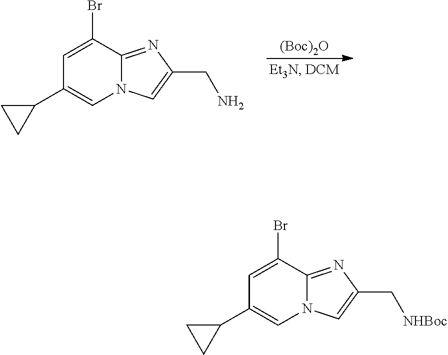 [SMILES: "(Boc)2O" ; NCc1cn2cc(C3CC3)cc(Br)c2n1 ; CC(C)(C)OC(=O)NCc1cn2cc(C3CC3)cc(Br)c2n1]

A mixture of (8-bromo-6-cyclopropylimidazo[1,2-a]pyridin-2-yl)methanamine (550 mg, 2.08 mmol), (Boc)₂O (679 mg, 3.11 mmol) and Et₃N (630 mg, 6.24 mmol) in DCM (20 mL) was stirred at room temperature for 16 h. The mixture was quenched with water (50 mL) and extracted with DCM (3×50 mL). The combined organics were washed with brine (50 mL), dried over Na₂SO₄ and concentrated in vacuo. The residue was purified by preparative TLC (eluent: DCM/MeOH=20/1) to give tert-butyl ((8-bromo-6-cyclopropylimidazo[1,2-a]pyridin-2-yl)methyl)carbamate (520 mg, 69%) as a yellow solid. ESI-MS [M+H]⁺: 366.2.

### Synthesis of 1-(2-(aminomethyl)-6-cyclopropylimidazo[1,2-a]pyridin-8-yl)-3-methylimidazolidine-2,4-dione

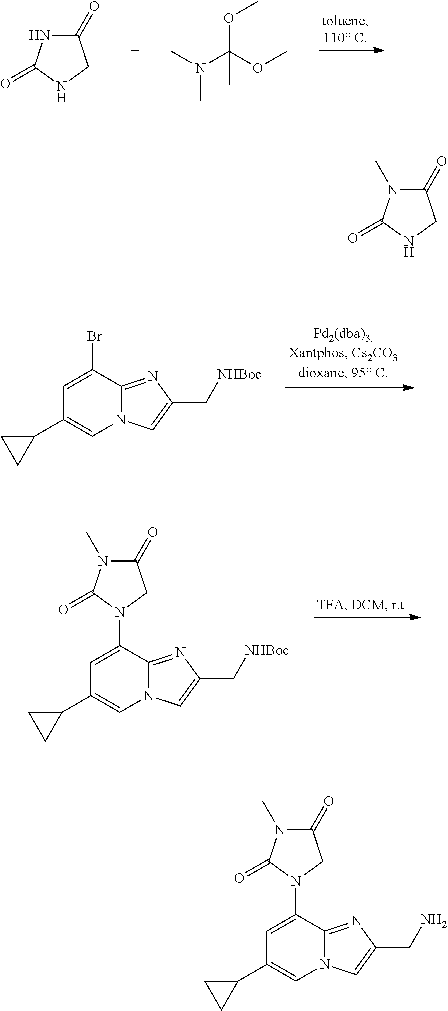

### Synthesis of 3-methylimidazolidine-2,4-dione

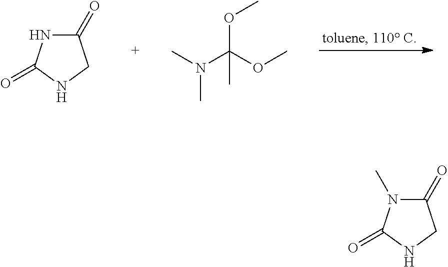

To a reaction mixture of imidazolidine-2,4-dione (1 g, 10 mmol) in dry toluene (25 mL) was added 1,1-dimethoxy-N,N-dimethylethan-1-amine (4 g, 30 mmol). The reaction was stirred at 110° C. for 3 h under N₂. The reaction was cooled to 0° C. for 15 minutes and filtered. The organic layers were concentrated and purified by silica gel chromatography (eluent: DCM/MeOH=10/1) to give 3-methylimidazolidine-2,4-dione (400 mg, 34%) as a white solid.

ESI-MS [M+H]⁺: 115.1.

### Synthesis of tert-butyl ((6-cyclopropyl-8-(3-methyl-2,4-dioxoimidazolidin-1-yl)imidazo[1,2-a]pyridin-2-yl)methyl)carbamate

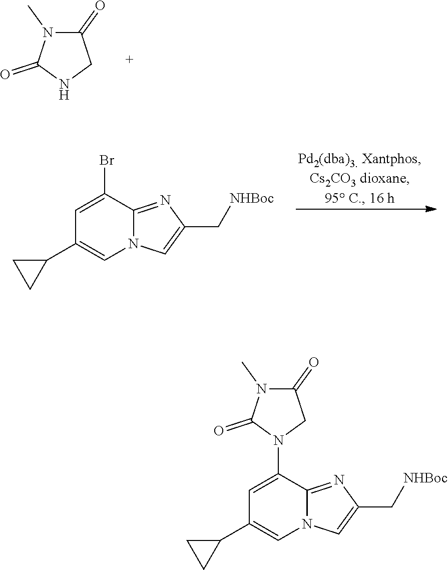

To a mixture of 3-methylimidazolidine-2,4-dione (224 mg, 2 mmol), tert-butyl ((8-bromo-6-cyclopropylimidazo[1,2-a]pyridin-2-yl)methyl)carbamate (365 mg, 1 mmol) and Cs₂CO₃ (975 mg, 3 mmol) in dry dioxane (10 mL) was added Pd₂(dba)₃ (183 mg, 0.2 mmol) and XantPhos (116 mg, 0.2 mmol). The reaction was stirred at 100° C. for 16 h under N₂. The reaction was quenched with water (50 mL) and extracted with EtOAc (30 mL×3). The organic layers were washed with brine (30 mL), dried with Na₂SO₄, concentrated in vacuo and purified by silica gel chromatography (eluent: DCM/MeOH=10/1) to give tert-butyl ((6-cyclopropyl-8-(3-methyl-2,4-dioxoimidazolidin-1-yl)imidazo[1,2-a]pyridin-2-yl)methyl)carbamate (150 mg, 37%) as a yellow solid.

ESI-MS [M+H]⁺: 400.0.

### Synthesis of 1-(2-(aminomethyl)-6-cyclopropylimidazo[1,2-a]pyridin-8-yl)-3-methylimidazolidine-2,4-dione

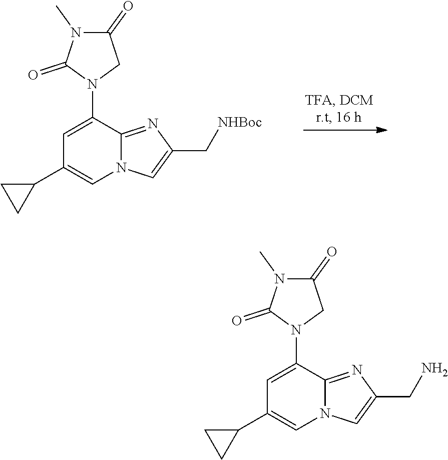 [SMILES: CN1C(=O)CN(c2cc(C3CC3)cn3cc(CNC(=O)OC(C)(C)C)nc23)C1=O ; CN1C(=O)CN(c2cc(C3CC3)cn3cc(CN)nc23)C1=O]

To a reaction mixture of tert-butyl ((6-cyclopropyl-8-(3-methyl-2,4-dioxoimidazolidin-1-yl)imidazo[1,2-a]pyridin-2-yl)methyl)carbamate (500 mg, 1.25 mmol) in dry DCM (10 mL) was added TFA (2 mL). The reaction was stirred at room temperature for 16 h under N₂. The reaction mixture was neutralized with NH₃ in MeOH then concentrated in vacuo and purified by silica gel chromatography (eluent: DCM/MeOH=10/1) to give 1-(2-(aminomethyl)-6-cyclopropylimidazo[1,2-a]pyridin-8-yl)-3-methylimidazolidine-2,4-dione (150 mg, 40%) as a white solid.

ESI-MS [M+H]⁺: 300.1.

### Synthesis of 6-cyclopropyl-8-(3-methyl-2,4-dioxoimidazolidin-1-yl)imidazo[1,2-a]pyridine-2-carbaldehyde

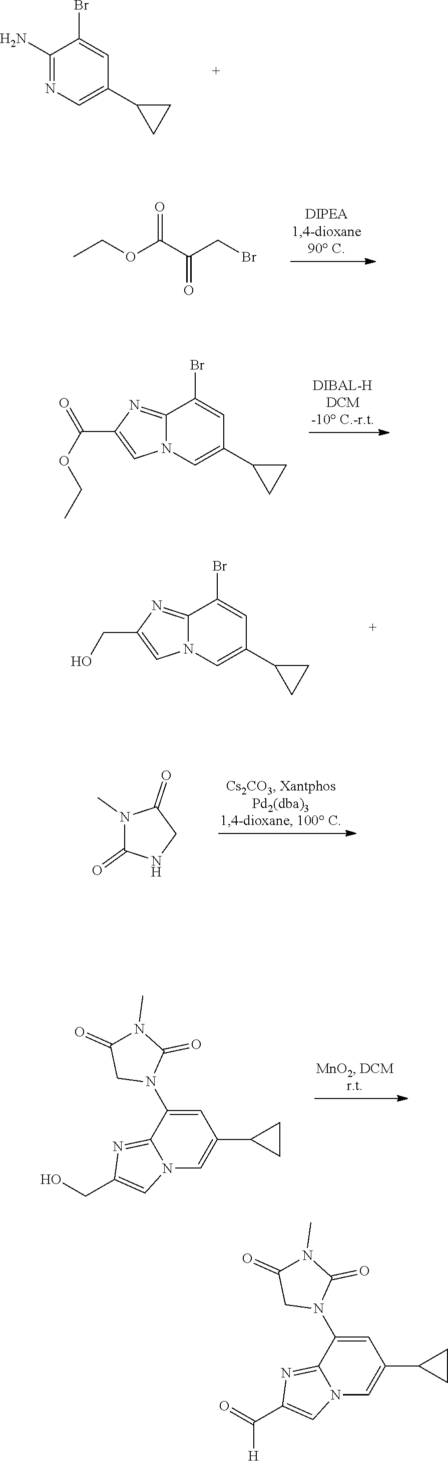

### Synthesis of 8-bromo-6-cyclopropyl-imidazo[1,2-a]pyridine-2-carboxylate

A mixture of 3-bromo-5-cyclopropyl-pyridin-2-amine (5.0 g, 24 mmol), ethyl bromopyruvate (4.9 mL, 35 mmol) and DIPEA (8.2 mL, 47 mmol) in 1,4-dioxane (200 mL) was stirred at 90° C. for 3 h. The mixture was allowed to cool and concentrated in vacuo. The residue was dissolved in DCM, washed with water, dried over MgSO₄, filtered and concentrated in vacuo. The residue was purified by silica gel column chromatography, eluting with 10-60% EtOAc in cyclohexane to give the title compound (4.9 g, 67%) as a brown solid.

ESI-MS (M+H)+: 309, ¹H NMR (400 MHz, DMSO) δ 8.58 (s, 1H), 8.47 (s, 1H), 7.54 (d, J=1.3 Hz, 1H), 4.37 (q, J=7.1 Hz, 2H), 2.07-1.98 (m, 1H), 1.38 (t, J=7.0 Hz, 3H), 1.04-0.97 (m, 3H), 0.83-0.76 (m, 2H).

### Synthesis of (8-bromo-6-cyclopropyl-imidazo[1,2-a]pyridin-2-yl)methanol

DIBAL-H (1.0 M in THF, 54 mL, 53.8 mmol) was added dropwise to a stirred solution of ethyl 8-bromo-6-cyclopropyl-imidazo[1,2-a]pyridine-2-carboxylate (4.8 g, 15 mmol) in DCM (95 mL) at −10° C. under a N₂ atmosphere. The mixture was warmed to room temperature over 2 h. Rochelle's salts (sat. aq.) were added and the mixture was stirred for 18 h. The mixture was extracted with DCM (3×) and the combined organics were dried over MgSO₄, filtered through Celite® and concentrated in vacuo to give the title compound (3.0 g, 73%) as an off-white solid which was used without further purification.

ESI-MS (M+H)+: 267, 269, ¹H NMR (400 MHz, DMSO) δ 8.40 (s, 1H), 7.81 (s, 1H), 7.33 (s, 1H), 5.25 (dd, J=5.7, 5.7 Hz, 1H), 4.59 (d, J=5.8 Hz, 2H), 2.00-1.91 (m, 1H), 0.97-0.90 (m, 2H), 0.76-0.70 (m, 2H).

### Synthesis of 1-(6-cyclopropyl-2-(hydroxymethyl)imidazo[1,2-a]pyridin-8-yl)-3-methylimidazolidine-2,4-dione

A mixture of (8-bromo-6-cyclopropyl-imidazo[1,2-a]pyridin-2-yl)methanol (2.44 g, 9.13 mmol, 3-methylimidazolidine-2,4-dione (1.20 g, 10 mmol), Pd₂(dba)₃ (420 mg, 0.46 mmol), Xantphos (530 mg, 0.91 mmol) and Cs₂CO₃ (8.9 g, 27 mmol) in 1,4-dioxane (110 mL) was degassed with N₂ and heated at 100° C. for 18 h. Water (200 mL) was added and the mixture was extracted into EtOAc (3×100 mL). The combined organic layers were dried (MgSO₄), filtered and concentrated in vacuo. The residue was purified by silica gel column chromatography, eluting with 0-10% MeOH in DCM to give the title compound (1.40 g, 51%) as a yellow/brown solid.

ESI-MS (M+H)+: 301, ¹H NMR (400 MHz, DMSO) δ 8.40 (s, 1H), 7.81 (s, 1H), 7.33 (s, 1H), 5.25 (dd, J=5.7, 5.7 Hz, 1H), 4.59 (d, J=5.8 Hz, 2H), 2.00-1.91 (m, 1H), 0.97-0.90 (m, 2H), 0.76-0.70 (m, 2H).

### Synthesis of 6-cyclopropyl-8-(3-methyl-2,4-dioxoimidazolidin-1-yl)imidazo[1,2-a]pyridine-2-carbaldehyde

MnO₂ (3.5 g, 40 mmol) was added to a stirred solution of 1-(6-cyclopropyl-2-(hydroxymethyl)imidazo[1,2-a]pyridin-8-yl)-3-methylimidazolidine-2,4-dione (1.2 g, 4.0 mmol) in DCM (40 mL) and the mixture was stirred at room temperature for 18 h. Further MnO₂ (3.5 g, 40 mmol) was added and the mixture was stirred at room temperature for 5 h. Further MnO₂ (690 mg, 7.8 mmol) was added and the mixture was stirred at room temperature for 18 h. The mixture was filtered through Celite® and concentrated in vacuo to give the title compound (800 mg, 67%) as an off-white solid.

ESI-MS (M+H)+: 299.2, ¹H NMR (400 MHz, DMSO) δ 10.03 (s, 1H), 8.60-8.59 (m, 1H), 8.38-8.37 (m, 1H), 7.53 (d, J=1.5 Hz, 1H), 4.93 (s, 2H), 3.00 (s, 3H), 2.06-1.98 (m, 1H), 1.04-0.98 (m, 2H), 0.74-0.69 (m, 2H).

### Synthesis of 1-(2-(chloromethyl)-6-cyclopropylimidazo[1,2-a]pyridin-8-yl)-3-methylimidazolidine-2,4-dione

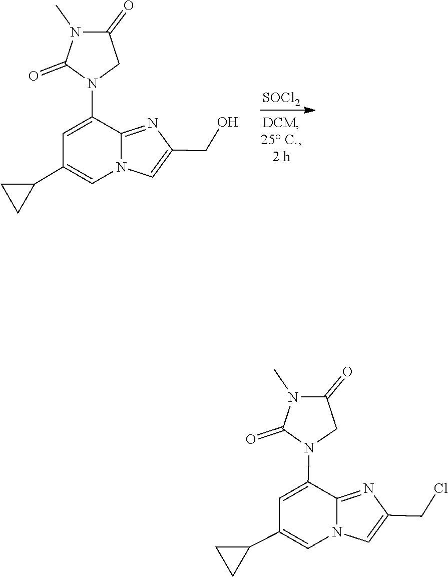 [SMILES: CN1C(=O)CN(c2cc(C3CC3)cn3cc(CO)nc23)C1=O ; "SOCl2" ; CN1C(=O)CN(c2cc(C3CC3)cn3cc(CCl)nc23)C1=O]

To a solution of 1-(6-cyclopropyl-2-(hydroxymethyl)imidazo[1,2-a]pyridin-8-yl)-3-methylimidazolidine-2,4-dione (0.40 g, 1.3 mmol) in DCM (10 mL) was added SOCl₂ (3.0 mL). The resulting mixture was stirred at 25° C. for 2 h under N₂. The reaction mixture was concentrated in vacuo to give 1-(2-(chloromethyl)-6-cyclopropylimidazo[1,2-a]pyridin-8-yl)-3-methylimidazolidine-2,4-dione (0.42 g, quant) as a yellow solid which was used to the next step without further purification. ESI-MS [M+H]+: 319.1.

### Synthesis of 1-(6-cyclopropyl-2-(1-hydroxyethyl)imidazo[1,2-a]pyridin-8-yl)-3-methylimidazolidine-2,4-dione

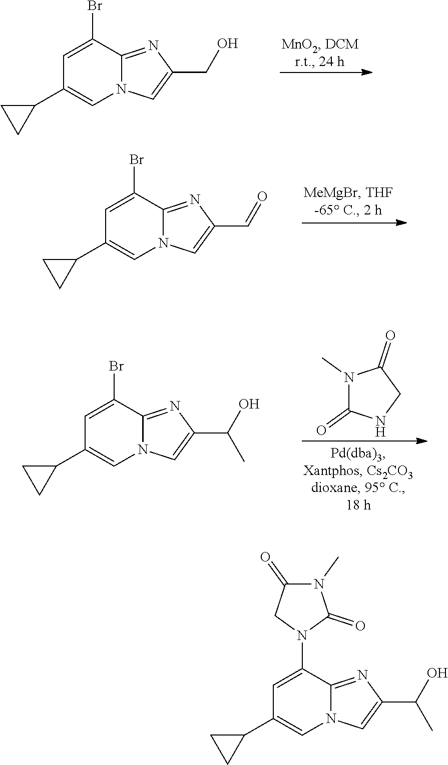

### Synthesis of 8-bromo-6-cyclopropylimidazo[1,2-a]pyridine-2-carbaldehyde

A mixture of (8-bromo-6-cyclopropylimidazo[1,2-a]pyridin-2-yl)methanol (10 g, 37.45 mmol) and MnO₂ (9.8 g, 112 mmol) was stirred at room temperature for 12 h. Second batch of MnO₂ (9.8 g, 112 mmol) was added and stirred at room temperature for another 12 h. The reaction mixture was filtered through a short silica pad and washed with EtOAc (500 mL). The filtrate was concentrated in vacuo to give 8-bromo-6-cyclopropylimidazo[1,2-a]pyridine-2-carbaldehyde (8 g, 80.8%) as a yellow solid. ESI-MS [M+H]⁺: 266.2.

### Synthesis of 1-(8-bromo-6-cyclopropylimidazo[1,2-a]pyridin-2-yl)ethan-1-ol

To a solution of 8-bromo-6-cyclopropylimidazo[1,2-a]pyridine-2-carbaldehyde (8 g, 30 mmol) in dry THF (100 mL) was added MeMgBr (30 mL, 3 M solution in Et₂O, 90 mmol) at −65° C. slowly and the resulting solution was stirred at −65° C. for 2 h. The reaction was quenched with saturated aqueous NH₄Cl (70 mL) at −65° C., stirred and warmed to room temperature then extracted with EtOAc (4×75 mL). The combined organic layers were washed with brine (75 mL), dried over Na₂SO₄ then concentrated in vacuo to give the crude product, which was purified with silica gel chromatography (eluent: DCM/MeOH=15/1) to give 1-(8-bromo-6-cyclopropylimidazo[1,2-a]pyridin-2-yl)ethan-1-ol (7.3 g, 86.9%) as a yellow solid. ESI-MS [M+H]⁺: 281.2.

### Synthesis of 1-(6-cyclopropyl-2-(1-hydroxyethyl)imidazo[1,2-a]pyridin-8-yl)-3-methylimidazolidine-2,4-dione

A mixture of 1-(8-bromo-6-cyclopropylimidazo[1,2-a]pyridin-2-yl)ethan-1-ol (7.3 g, 25.98 mmol), 3-methylimidazolidine-2,4-dione (8.9 g, 77.9 mmol), Pd₂(dba)₃ (2.38 g, 2.6 mmol), Xantphos (3 g, 5.2 mmol) and Cs₂CO₃ (25 g, 77 mmol) in dioxane (75 mL) was stirred at 95° C. for 18 h. The reaction mixture was cooled to room temperature, filtered through Celite®, washed with EtoAc (3×75 mL). The combined filtrate was washed with brine (80 mL), dried over Na₂SO₄ then concentrated in vacuo to give the crude product, which was purified with silica gel chromatography (eluent: DCM/MeOH=15/1) to give 1-(6-cyclopropyl-2-(1-hydroxyethyl)imidazo[1,2-a]pyridin-8-yl)-3-methylimidazolidine-2,4-dione (6.1 g, 75%) as a yellow solid. ESI-MS [M+H]⁺: 315.2.

### Synthesis of 1-(6-cyclopropyl-2-(hydroxymethyl)imidazo[1,2-a]pyrazin-8-yl)-3-methylimidazolidine-2,4-dione

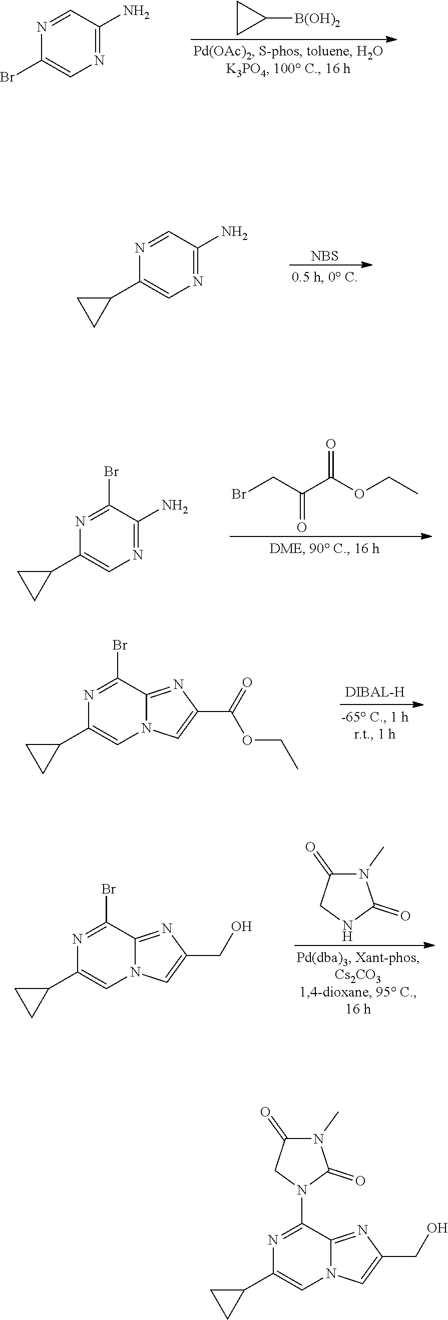

### Synthesis of 5-cyclopropylpyrazin-2-amine

To a mixture of 5-bromopyrazin-2-amine (10 g, 57 mmol) in toluene/H₂O (V/V=1/1, 400 mL) were added cyclopropylboronic acid (15 g, 0.17 mol), S-phos (2.4 g, 5.8 mmol), K₃PO₄ (36 g, 0.17 mol) and Pd(OAc)₂ (1.3 g, 5.8 mmol). The mixture was stirred at 100° C. for 16 h under N₂ and then cooled to room temperature. The reaction mixture was filtered through Celite® and the filter cake was washed with DCM/MeOH (10/1, 100 mL). The filtrate was concentrated in vacuo to give the crude, which was purified by silica gel chromatography (eluent: PE/EtOAc=10/1˜5/1) to give 5-cyclopropylpyrazin-2-amine (5.6 g, 72%) as a yellow solid. ESI-MS [M+H]⁺: 136.1.

### Synthesis of 3-bromo-5-cyclopropylpyrazin-2-amine

To a stirred solution of 5-cyclopropylpyrazin-2-amine (5.6 g, 41 mmol) in CH₃CN (40 mL) was added NBS (7.4 g, 42 mmol) at 0° C. The mixture was stirred at room temperature for 30 min. The reaction mixture was poured into water (50 mL) and extracted with EtOAc (3×50 mL). The combined organics were washed with saturated brine solution (100 mL) and dried over Na₂SO₄. The organic layers were concentrated and purified by column chromatography on silica gel (eluent: PE/EtOAc=10/1) to give 3-bromo-5-cyclopropylpyrazin-2-amine (2.0 g, yield 23%) ESI-MS [M+H]+: 214.0

### Synthesis of ethyl 8-bromo-6-cyclopropylimidazo[1,2-a]pyrazine-2-carboxylate

To a mixture of 3-bromo-5-cyclopropylpyrazin-2-amine (2.0 g, 9.3 mmol) in DME (30 mL) was added ethyl 3-bromo-2-oxopropanoate (2.0 g, 10 mmol). The mixture was stirred at 90° C. for 16 h and cooled to room temperature. Then the mixture was poured into water (50 mL) and extracted with EtOAc (3×50 mL). The combined organics were washed with saturated brine solution (100 mL), dried over Na₂SO₄ and concentrated in vacuo. The residue was purified by column chromatography on silica gel (eluent: PE/EtOAc=2/1) to give ethyl 8-bromo-6-cyclopropylimidazo[1,2-a]pyrazine-2-carboxylate (1.0 g, 35%). ESI-MS [M+H]+: 310.0

### Synthesis of (8-bromo-6-cyclopropylimidazo[1,2-a]pyrazin-2-yl)methanol

To a mixture of 8-bromo-6-cyclopropylimidazo[1,2-a]pyrazine-2-carboxylate (1.0 g, 3.2 mmol) in THF (20 mL) was added DIBAL-H (11 mL, 11 mmol) at −65° C. The mixture was stirred at −65° C. for 1 h and then warmed to 25° C. and stirred for another 1 h. The reaction mixture was poured into ice-water (20 mL) and extracted with EtOAc (3×50 mL). The combined organics were washed with saturated brine solution (50 mL), dried over Na₂SO₄ and concentrated in vacuo to give a residue, which was purified by column chromatography on silica gel (eluent: PE/EtOAc=5/1) to give (8-bromo-6-cyclopropylimidazo[1,2-a]pyrazin-2-yl)methanol (0.49 g, yield 57%). ESI-MS [M+H]+: 268.0

### Synthesis of 1-(6-cyclopropyl-2-(hydroxymethyl)imidazo[1,2-a]pyrazin-8-yl)-3-methylimidazolidine-2,4-dione

To a mixture of (8-bromo-6-cyclopropylimidazo[1,2-a]pyrazin-2-yl)methanol (0.49 g, 1.8 mmol) in dioxane (15 mL) were added 3-methylimidazolidine-2,4-dione (0.83 g, 7.3 mmol), Pd₂(dba)₃ (0.34 g, 0.37 mmol), Xant-Phos (0.38 g, 0.66 mmol), Cs₂CO₃ (1.8 g, 5.5 mmol). After stirring at 90° C. for 16 h under N₂, the reaction mixture was filtered and the filter cake was washed with DCM/MeOH (10/1, 50 mL). The filtrate was concentrated in vacuo to give the crude, which was purified by column chromatography on silica gel (eluent: DCM/MeOH=30/1˜10/1) to give 1-(6-cyclopropyl-2-(hydroxymethyl)imidazo[1,2-a]pyrazin-8-yl)-3-methylimidazolidine-2,4-dione (0.40 g, 74%). ESI-MS [M+H]+: 302.1

### Synthesis of 7-bromo-4-chloro-2-((1S,2S)-2-(4-methylpyrimidin-2-yl)cyclopropyl)quinoline

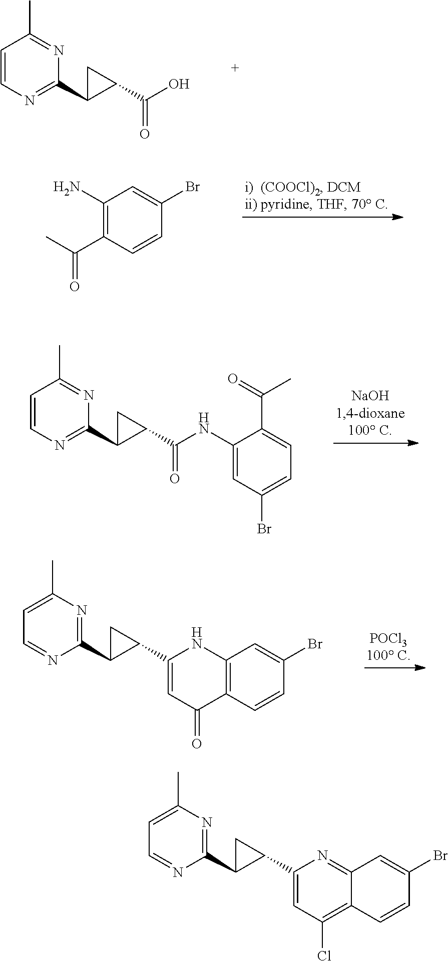

### Synthesis of (1S,2S)—N-(2-acetyl-5-bromophenyl)-2-(4-methylpyrimidin-2-yl)cyclopropane-1-carboxamide

Oxalyl chloride (0.98 mL, 11 mmol) was added dropwise to a stirred solution of (1S,2S)-2-(4-methylpyrimidin-2-yl)cyclopropane-1-carboxylic acid (1.00 g, 5.6 mmol) and DMF (0.050 mL) in THF (10 mL) at 0° C. and stirred at 0° C. for 30 min. The reaction was then concentrated in vacuo. The residue was suspended in THF (20 mL), 1-(2-amino-4-bromo-phenyl)ethanone (1300 mg, 5.9 mmol) and pyridine (4.5 mL, 56 mmol) were added and the mixture was heated at 70° C. for 18 h. The mixture was cooled to room temperature, quenched with water (50 mL) and the precipitate formed was filtered, washed with water, dried over MgSO₄ and concentrated in vacuo to give the title compound (1.7 g, 85%) as a dark brown solid.

ESI-MS (M+H)+: 374.0, 376.0, ¹H NMR (400 MHz, DMSO) δ 11.55 (s, 1H), 8.60 (d, J=5.1 Hz, 1H), 8.53 (d, J=2.0 Hz, 1H), 7.95 (d, J=8.6 Hz, 1H), 7.48 (dd, J=2.0, 8.3 Hz, 1H), 7.28 (d, J=5.1 Hz, 1H), 2.68-2.61 (m, 4H), 2.41-2.35 (m, 1H), 1.65-1.59 (m, 2H).

### Synthesis of 7-bromo-2-((1S,2S)-2-(4-methylpyrimidin-2-yl)cyclopropyl)quinolin-4 (1H)-one

NaOH (440 mg, 11 mmol) was azetroped with toluene (3×) then added to stirred suspension of (1S,2S)—N-(2-acetyl-5-bromophenyl)-2-(4-methylpyrimidin-2-yl)cyclopropane-1-carboxamide (1.70 g, 4.5 mmol) in 1,4-dioxane (20 mL). The reaction was heated at 100° C. for 1 h. The reaction was cooled to room temperature and poured into NH₄Cl (sat. aq., 50 mL). The resulting precipitate was filtered and dried under high vacuum to give the title compound (1.3 g, 77%) as a dark brown solid.

¹H NMR (400 MHz, DMSO) δ 11.71-11.71 (m, 1H), 8.58 (d, J=5.1 Hz, 1H), 7.96 (d, J=8.7 Hz, 1H), 7.75 (s, 1H), 7.44 (dd, J=1.7, 8.6 Hz, 1H), 5.94 (s, 1H), 2.71-2.64 (m, 1H), 2.59-2.54 (m, 1H), 2.45 (s, 3H), 1.83-1.71 (m, 2H).

### Synthesis of 7-bromo-4-chloro-2-((1S,2S)-2-(4-methylpyrimidin-2-yl)cyclopropyl)quinoline

7-bromo-2-((1S,2S)-2-(4-methylpyrimidin-2-yl)cyclopropyl)quinolin-4(H)-one (1.30 g, 3.65 mmol) was stirred in POCl₃ (8.5 mL, 91.2 mmol) at 100° C. for 90 min, cooled to room temperature for 30 min then stirred at 100° C. for 30 min. The mixture was cooled and concentrated in vacuo. The residue was dissolved in DCM (500 mL), washed with NaHCO₃ (sat. aq., 150 mL), water (100 mL), brine (sat. aq., 150 mL), water (100 mL), dried over MgSO₄, filtered and concentrated in vacuo. The residue was purified by silica gel column chromatography, eluting with 0-100% EtOAc in cyclohexane to give the title compound (385 mg, 28%) as an orange solid.

ESI-MS (M+H)+: 376.1, 374.1, ¹H NMR (400 MHz, DMSO) δ 8.56 (1H, d, J=5.1 Hz), 8.22-8.21 (1H, m), 8.09 (1H, d, J=8.8 Hz), 7.96 (1H, s), 7.83 (1H, dd, J=2.0, 8.9 Hz), 7.23-7.22 (1H, m), 2.93-2.84 (2H, m), 2.44 (3H, s), 1.92-1.82 (2H, m).

### Synthesis of 4-(azetidin-1-yl)-7-bromo-2-((1S,2S)-2-(4-methylpyrimidin-2-yl)cyclopropyl)quinoline

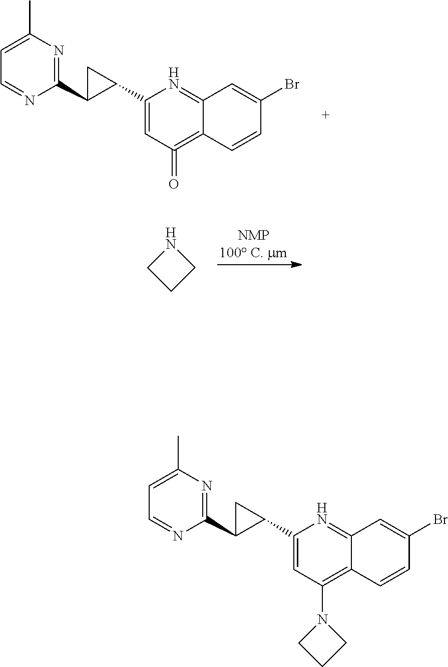

### Synthesis of 4-(azetidin-1-yl)-7-bromo-2-((1S,2S)-2-(4-methylpyrimidin-2-yl)cyclopropyl)quinoline

A solution of 7-bromo-4-chloro-2-[(1S,2S)-2-(4-methylpyrimidin-2-yl)cyclopropyl]quinoline (120 mg, 0.32 mmol) and azetidine (0.11 mL, 1.6 mmol) in NMP (0.50 mL) was stirred at 140° C. in a microwave reactor for 35 min. The mixture was diluted with DCM, water and brine (sat. aq.) to give an emulsion. The mixture was concentrated in vacuo, suspended in DCM, filtered and concentrated in vacuo. The residue was loaded onto an SCX cartridge, washed with MeOH in DCM and eluted with (7N NH₃ in MeOH) in DCM to give the title compound (90 mg, 71%) as a yellow solid. ESI-MS (M+H)+: 397.2, 395.2, ¹H NMR (400 MHz, DMSO) δ 8.55 (d, J=5.0 Hz, 1H), 7.95-7.90 (m, 2H), 7.52-7.49 (m, 1H), 7.22 (d, J=5.1 Hz, 1H), 6.30 (s, 1H), 4.47-4.41 (m, 4H), 2.86-2.82 (m, 1H), 2.73-2.67 (m, 1H), 2.45-2.43 (m, 5H), 1.96-1.85 (m, 1H), 1.77-1.73 (m, 1H).

The compounds in Table 1 were synthesised in a similar manner to that described for 7-bromo-4-chloro-2-((1S,2S)-2-(4-methylpyrimidin-2-yl)cyclopropyl)quinoline from 7-bromo-4-chloro-2-[(1S,2S)-2-(4-methylpyrimidin-2-yl)cyclopropyl]quinoline and an appropriate amine coupling partner.

TABLE 1

| Compound | Coupling partner | Analytical data |
|---|---|---|
| 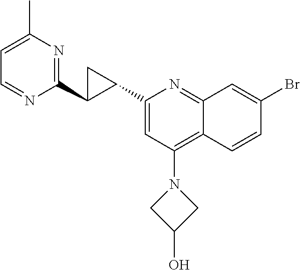 [SMILES: Cc1ccnc([C@H]2C[C@@H]2c2cc(N3CC(O)C3)c3ccc(Br)cc3n2)n1] | 3-azetidinol | ESI-MS (M + H)+: 411.2, 413.2 |
| 1-(7-bromo-2-((1S,2S)-2-(4- | | |
| methylpyrimidin-2- | | |
| yl)cyclopropyl)quinolin-4-yl)azetidin- | | |
| 3-ol | | |
| 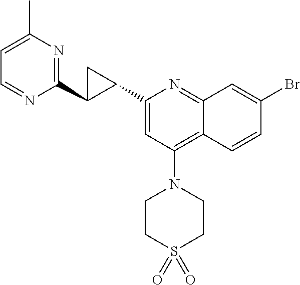 [SMILES: Cc1ccnc([C@H]2C[C@@H]2c2cc(N3CCS(=O)(=O)CC3)c3ccc(Br)cc3n2)n1] | thiomorpholine 1,1- dioxide | ESI-MS (M + H)+: 473.3, 475.3, ¹H NMR (400 MHz, DMSO) δ 8.53 (d, J = 5.1 Hz, 1H), 8.05 (d, J = 2.0 Hz, 1H), 8.01-7.94 (m, 1H), 7.59 (dd, J = 2.1, 8.9 Hz, 1H), 7.27 (s, 1H), 7.21-7.17 (m, 1H), 3.62 (s, 4H), 3.43 (s, 4H), 2.81 (dd, J = 6.5, 6.5 Hz, 2H), 2.42 (s, 3H), 1.86-1.75 (m, 2H). |
| 4-(7-bromo-2-((1S,2S)-2-(4- | | |
| methylpyrimidin-2-yl)cyclopropyl) | | |
| quinolin-4-yl)thiomorpholine 1,1- | | |
| dioxide | | |
| 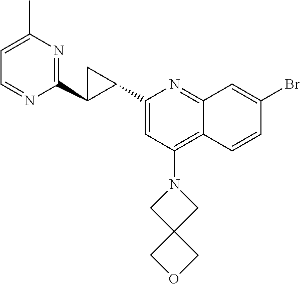 [SMILES: Cc1ccnc([C@H]2C[C@@H]2c2cc(N3CC4(COC4)C3)c3ccc(Br)cc3n2)n1] | 2-oxa-6- azaspiro[3.3] heptane | ESI-MS (M + H)+: 439.3, 437.3, ¹H NMR (400 MHz, DMSO) δ 8.53 (1H, d, J = 5.1 Hz), 7.91- 7.89 (1H, m), 7.85 (1H, d, J = 8.9 Hz), 7.44 (1H, dd, J = 2.2, 9.0 Hz), 7.19 (1H, d, J = 5.1 Hz), 6.41 (1H, s), 4.76 (4H, s), 4.52 (4H, s), 2.78-2.72 (1H, m), 2.69- 2.63 (1H, m), 2.43 (3H, s), 1.83- 1.77 (1H, m), 1.74-1.68 (1H, m). |
| 6-(7-bromo-2-((1S,2S)-2-(4- | | |
| methylpyrimidin-2- | | |
| yl)cyclopropyl)quinolin-4-yl)-2-oxa-6- | | |
| azaspiro[3.3]heptane | | |
| 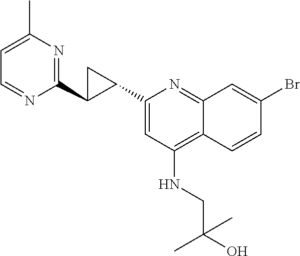 [SMILES: Cc1ccnc([C@H]2C[C@@H]2c2cc(NCC(C)(C)O)c3ccc(Br)cc3n2)n1] | 1-amino-2-methyl- 2-propanol | ESI-MS (M + H)+: 429.3, 427.3. |
| 1-((7-bromo-2-((1S,2S)-2-(4- | | |
| methylpyrimidin-2- | | |
| yl)cyclopropyl)quinolin-4-yl)amino)- | | |
| 2-methylpropan-2-ol | | |
| 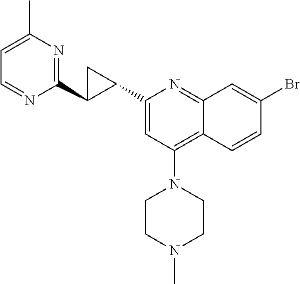 [SMILES: Cc1ccnc([C@H]2C[C@@H]2c2cc(N3CCN(C)CC3)c3ccc(Br)cc3n2)n1] | 1-methylpiperazine | ESI-MS (M + H)+: 438.3, 440.3, ¹H NMR (400 MHz, CDCl₃) δ 8.44 (1H, d, J = 5.1 Hz), 8.10 (1H, d, J = 1.9 Hz), 7.78 (1H, d, J = 8.9 Hz), 7.44 (1H, dd, J = 2.0, 8.9 Hz), 6.94 (1H, d, J = 5.3 Hz), 6.79 (1H, s), 3.21 (4H, s), 2.97- 2.91 (1H, m), 2.85-2.76 (1H, m), 2.72-2.66 (4H, m), 2.48 (3H, s), 2.41 (3H, s), 1.99-1.94 (1H, m), 1.90-1.84 (1H, m) |
| 7-bromo-4-(4-methylpiperazin-1-yl)- | | |
| 2-((1S,2S)-2-(4-methylpyrimidin-2- | | |
| yl)cyclopropyl)quinoline | | |

### Synthesis of rac-7-bromo-4-chloro-2-((1S*,2S*)-2-(4-methylpyrimidin-2-yl)cyclopropyl)quinolin

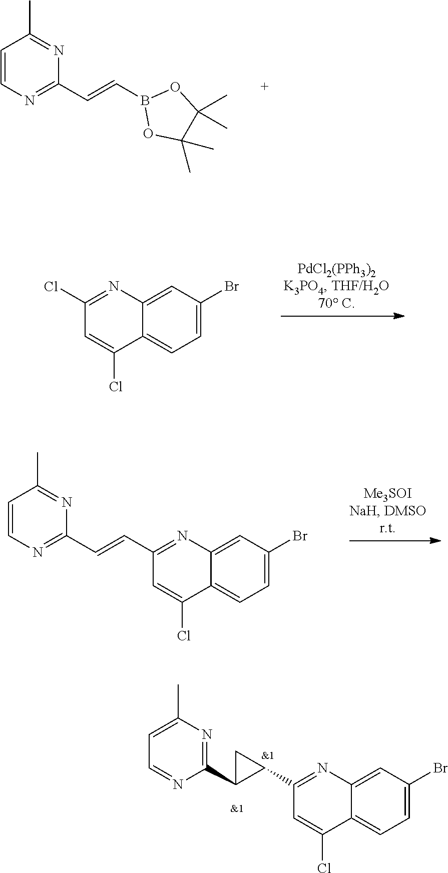 [SMILES: Cc1ccnc(/C=C/B2OC(C)(C)C(C)(C)O2)n1 ; Clc1cc(Cl)c2ccc(Br)cc2n1 ; Cc1ccnc(/C=C/c2cc(Cl)c3ccc(Br)cc3n2)n1 ; Cc1ccnc([C@H]2C[C@@H]2c2cc(Cl)c3ccc(Br)cc3n2)n1]

### Synthesis of (E)-7-bromo-4-chloro-2-(2-(4-methylpyrimidin-2-yl)vinyl)quinoline

A mixture of (E)-4-methyl-2-(2-(4,4,5,5-tetramethyl-1,3,2-dioxaborolan-2-yl)vinyl)pyrimidine (110 mg, 0.41 mmol), 7-bromo-2,4-dichloroquinoline (100 mg, 0.41 mmol) and K₃PO₄ (170 mg, 0.81 mmol) in THF (4.0 mL) and water (1.0 mL) was degassed with N₂ then PdCl₂(PPh₃)₂ (14 mg, 0.020 mmol) was added. The mixture was heated at 70° C. for 18 h. The mixture was diluted with water and extracted with EtOAc. The organic layer was dried over MgSO₄, filtered and concentrated in vacuo. The residue was purified by silica gel column chromatography, eluting with 0-30% EtOAc in cyclohexane to give the title compound (67 mg, 460%) as a yellow solid.

¹H NMR (400 MHz, DMSO) δ 8.73 (d, J=5.1 Hz, 1H), 8.37-8.34 (m, 2H), 8.13 (d, J=8.9 Hz, 1H), 8.04 (d, J=16.3 Hz, 1H), 7.90 (dd, J=2.0, 9.0 Hz, 1H), 7.86 (d, J=16.1 Hz, 1H), 7.33 (d, J=5.0 Hz, 1H), 2.53 (s, 3H).

### Synthesis of rac-7-bromo-4-chloro-2-((1S*,2S*)-2-(4-methylpyrimidin-2-yl)cyclopropyl)quinoline

NaH (60% in mineral oil, 140 mg, 3.6 mmol) was added to a stirred solution of Me₃SOI (850 mg, 3.9 mmol) in DMSO (6.0 mL) and the mixture was stirred at room temperature under a N₂ atmosphere for 1 h. A slurry of (E)-7-bromo-4-chloro-2-(2-(4-methylpyrimidin-2-yl)vinyl)quinoline (470 mg, 1.3 mmol) in DMSO (7.0 mL) and THF (3.0 mL) was added and the mixture was stirred at room temperature for 3 h. The mixture was quenched with NH₄Cl (sat. aq., 75 mL) and the mixture was extracted with EtOAc (3×75 mL). The combined organics were dried over MgSO₄, filtered and concentrated in vacuo. The residue was purified by silica gel column chromatography, eluting with 0-50% EtOAc in cyclohexane to give the title compound (210 mg, 44%) as a colourless gum.

ESI-MS (M+H)+: 376.1, 374.1, ¹H NMR (400 MHz, DMSO) δ 8.57-8.55 (m, 1H), 8.22-8.21 (m, 1H), 8.11-8.08 (m, 1H), 7.97 (s, 1H), 7.85-7.81 (m, 1H), 7.22 (d, J=5.0 Hz, 1H), 2.94-2.84 (m, 2H), 2.45-2.44 (m, 3H), 1.92-1.83 (m, 2H).

### Synthesis of rac-7-bromo-N,N-dimethyl-2-((1S*,2S*)-2-(4-methylpyrimidin-2-yl)cyclopropyl)quinolin-4-amine

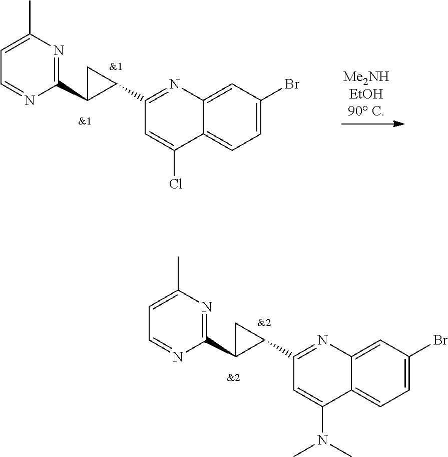 [SMILES: Cc1ccnc([C@H]2C[C@@H]2c2cc(Cl)c3ccc(Br)cc3n2)n1 ; Cc1ccnc([C@H]2C[C@@H]2c2cc(N(C)C)c3ccc(Br)cc3n2)n1]

Me₂NH (5.6 M in EtOH, 1.0 mL, 5.66 mmol) was added to a stirred mixture of rac-7-bromo-4-chloro-2-((1S*,2S*)-2-(4-methylpyrimidin-2-yl)cyclopropyl)quinoline (210 mg, 0.57 mmol) in EtOH (0.57 mL) and the mixture was sealed and stirred at 90° C. for 18 h. The mixture was cooled and further Me₂NH (5.6 M in EtOH, 1.0 mL, 5.66 mmol) was added. The mixture was sealed and stirred at 90° C. for 2 h. The mixture was concentrated in vacuo and the residue was purified by column chromatography on silica gel, eluting with 1-20% MeOH in DCM to give the title compound (140 mg, 63%) as a colourless gum.

ESI-MS (M+H)+: 385.2, 383.2, ¹H NMR (400 MHz, CDCl₃) δ 8.45 (d, J=5.1 Hz, 1H), 8.09 (d, J=2.0 Hz, 1H), 7.83 (d, J=8.9 Hz, 1H), 7.45-7.41 (m, 1H), 6.95-6.93 (m, 1H), 6.70 (s, 1H), 2.98 (s, 6H), 2.96-2.90 (m, 1H), 2.81-2.76 (m, 1H), 2.48 (s, 3H), 1.99-1.93 (m, 1H), 1.89-1.84 (m, 1H).

### Synthesis of rac-7-bromo-2-((1S*,2S*)-2-(4-methylpyrimidin-2-yl)cyclopropyl)-4-(oxetan-3-yloxy)quinoline

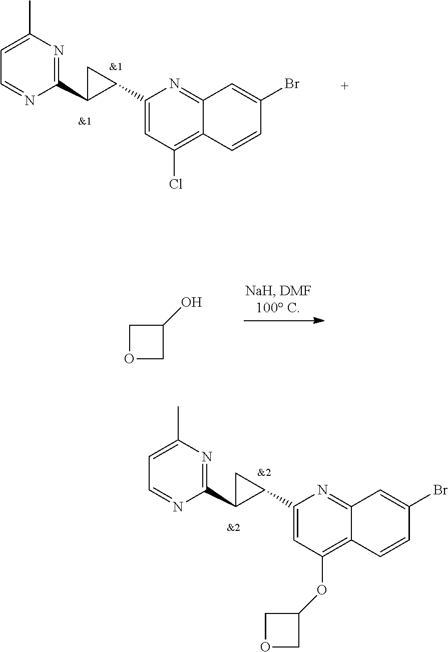 [SMILES: Cc1ccnc([C@H]2C[C@@H]2c2cc(Cl)c3ccc(Br)cc3n2)n1 ; OC1COC1 ; Cc1ccnc([C@H]2C[C@@H]2c2cc(OC3COC3)c3ccc(Br)cc3n2)n1]

3-Hydroxyoxetane (17 μL, 0.27 mmol) was added to a stirred suspension of rac-7-bromo-4-chloro-2-((1S*,2S*)-2-(4-methylpyrimidin-2-yl)cyclopropyl)quinoline (100 mg, 0.267 mmol) and NaH (60% in mineral oil, 27 mg, 0.67 mmol) in DMF (1.0 mL) and stirred at 100° C. under a N₂ atmosphere for 18 h. The mixture was quenched with water and extracted with DCM (3×). The combined organics were passed through a hydrophobic frit and concentrated in vacuo. The aqueous phase was further extracted with EtOAc (3×) and the combined organics were dried over MgSO₄, filtered, combined with the DCM residue and concentrated in vacuo to give the title compound (97 mg, 89%) as a brown solid which was used without further purification.

ESI-MS (M+H)+: 412, 414, ¹H NMR (400 MHz, DMSO) δ 8.59 (d, J=5.1 Hz, 1H), 8.15 (d, J=8.8 Hz, 1H), 8.10 (d, J=1.8 Hz, 1H), 7.70 (dd, J=2.0, 8.8 Hz, 1H), 7.25 (d, J=5.1 Hz, 1H), 6.91 (s, 1H), 5.64-5.58 (m, 1H), 5.15-5.07 (m, 2H), 4.77-4.71 (m, 2H), 2.91-2.83 (m, 2H), 2.48 (s, 3H), 1.91-1.80 (m, 2H).

### Synthesis of rac-7-bromo-4-((1-methylpiperidin-4-yl)oxy)-2-((1S*,2S*)-2-(4-methylpyrimidin-2-yl)cyclopropyl)quinoline

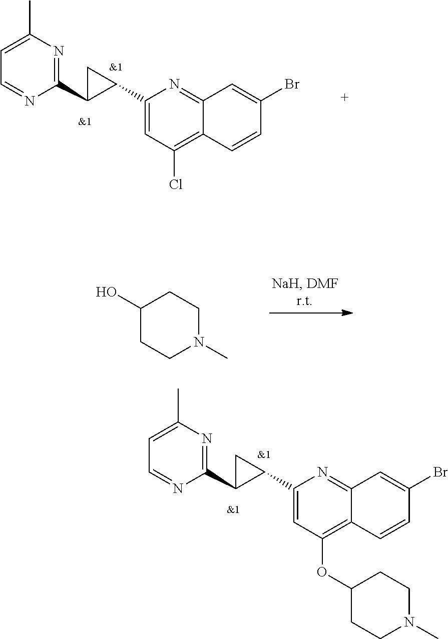

To a solution of N-methyl-4-piperidinol (0.016 mL, 0.13 mmol) in DMF (0.50 mL) under a N₂ atmosphere at room temperature was added NaH (60% in mineral oil, 13 mg, 0.33 mmol). After 10 min rac-7-bromo-4-chloro-2-((1S*,2S*)-2-(4-methylpyrimidin-2-yl)cyclopropyl)quinoline (50 mg, 0.13 mmol) was added. The mixture was stirred at room temperature for 2 h under a N₂ atmosphere. Further NaH (60% in mineral oil, 13 mg, 0.33 mmol) was added and the mixture was stirred at room temperature for 18 h under a N₂ atmosphere. The mixture was quenched with water and extracted with DCM (3×). The combined organics were passed through a hydrophobic frit and concentrated in vacuo. The above procedure was repeated then the residues from both reactions were combined and loaded onto an SCX cartridge, which was washed with MeOH. The compound was released using 7N NH₃ in MeOH and concentrated in vacuo to give the title compound (93 mg, 77%) as a yellow gum.

ESI-MS (M+H)+: 453, 455.

### Synthesis of rac-N-(7-bromo-2-((1S*,2S*)-2-(4-methylpyrimidin-2-yl)cyclopropyl)quinolin-4-yl)-N-methylmethanesulfonamide

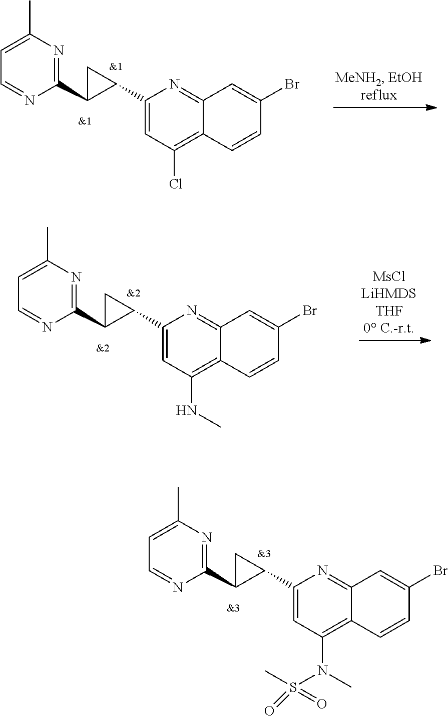 [SMILES: Cc1ccnc([C@H]2C[C@@H]2c2cc(Cl)c3ccc(Br)cc3n2)n1 ; CNc1cc([C@H]2C[C@@H]2c2nccc(C)n2)nc2cc(Br)ccc12 ; Cc1ccnc([C@H]2C[C@@H]2c2cc(N(C)S(C)(=O)=O)c3ccc(Br)cc3n2)n1]

### Synthesis of rac-7-bromo-N-methyl-2-((1S*,2S*)-2-(4-methylpyrimidin-2-yl)cyclopropyl)quinolin-4-amine

MeNH₂ (33% in EtOH, 0.83 mL, 6.7 mmol) was added to a stirred mixture of rac-7-bromo-4-chloro-2-((1S*,2S*)-2-(4-methylpyrimidin-2-yl)cyclopropyl)quinoline (250 mg, 0.67 mmol) in EtOH (1.0 mL). The mixture was sealed and heated at reflux for 24 h. The reaction mixture was concentrated in vacuo and the residue was purified by silica gel column chromatography, eluting with 0-10% (7 N NH₃ in MeOH) in DCM to give the title compound (160 mg, 65%) as a pale yellow solid.

ESI-MS (M+H)+: 369.1, 371.1, ¹H NMR (400 MHz, CDCl₃) δ 8.44 (d, J=5.1 Hz, 1H), 8.05 (d, J=1.8 Hz, 1H), 7.48 (d, J=8.8 Hz, 1H), 7.40 (dd, J=1.9, 8.7 Hz, 1H), 6.93 (d, J=5.1 Hz, 1H), 6.33 (s, 1H), 4.95-4.93 (m, 1H), 3.01 (d, J=4.8 Hz, 3H), 2.95-2.90 (m, 1H), 2.81-2.74 (m, 1H), 2.47 (s, 3H), 1.97-1.82 (m, 2H).

### Synthesis of rac-N-(7-bromo-2-((1S*,2S*)-2-(4-methylpyrimidin-2-yl)cyclopropyl)quinolin-4-yl)-N-methylmethanesulfonamide

LiHMDS (1.0 M in THF, 0.49 mL, 0.49 mmol) was added dropwise to a stirred solution of rac-7-bromo-N-methyl-2-((1S*,2S*)-2-(4-methylpyrimidin-2-yl)cyclopropyl)quinolin-4-amine (120 mg, 0.33 mmol) in THF (5.0 mL) at 0° C. After 15 min, MsCl (0.050 mL, 0.65 mmol) was added dropwise and the reaction mixture was warmed to room temperature and stirred for 18 h. The reaction mixture was quenched with water and extracted with DCM. The organic layer was dried over MgSO₄, filtered and concentrated in vacuo. The residue was purified by silica gel column chromatography, eluting with 0-100% EtOAc in cyclohexane to give the title compound (50 mg, 34%).

ESI-MS (M+H)+: 447.2, 449.2, ¹H NMR (400 MHz, CDCl₃) δ 8.46 (d, J=5.0 Hz, 1H), 8.20 (d, J=1.6 Hz, 1H), 7.96 (d, J=8.9 Hz, 1H), 7.63-7.59 (m, 1H), 7.35 (s, 1H), 6.97 (d, J=5.0 Hz, 1H), 3.37 (s, 3H), 3.03 (s, 3H), 3.03-2.95 (m, 1H), 2.87-2.82 (m, 1H), 2.49 (s, 3H), 2.06-1.91 (m, 2H).

### Synthesis of rac-4-(7-bromo-2-((1S*,2S*)-2-(4-methylpyrimidin-2-yl)cyclopropyl)quinolin-4-yl)morpholine

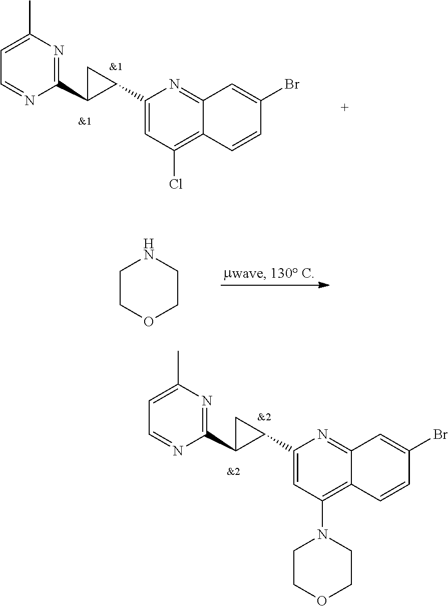

A mixture of rac-7-bromo-4-chloro-2-((1S*,2S*)-2-(4-methylpyrimidin-2-yl)cyclopropyl)quinoline (100 mg, 0.267 mmol) and morpholine (0.12 mL, 1.33 mmol) was heated in a microwave reactor to 140° C. for 2 periods of 30 min. The mixture was diluted with DCM then washed with water, dried over MgSO₄ and concentrated in vacuo to give the title compound which was used without further purification.

ESI-MS (M+H)+: 425.3, 427.3.

### Synthesis of (7-bromo-5-cyclopropylpyrazolo[1,5-a]pyridin-2-yl)methanol

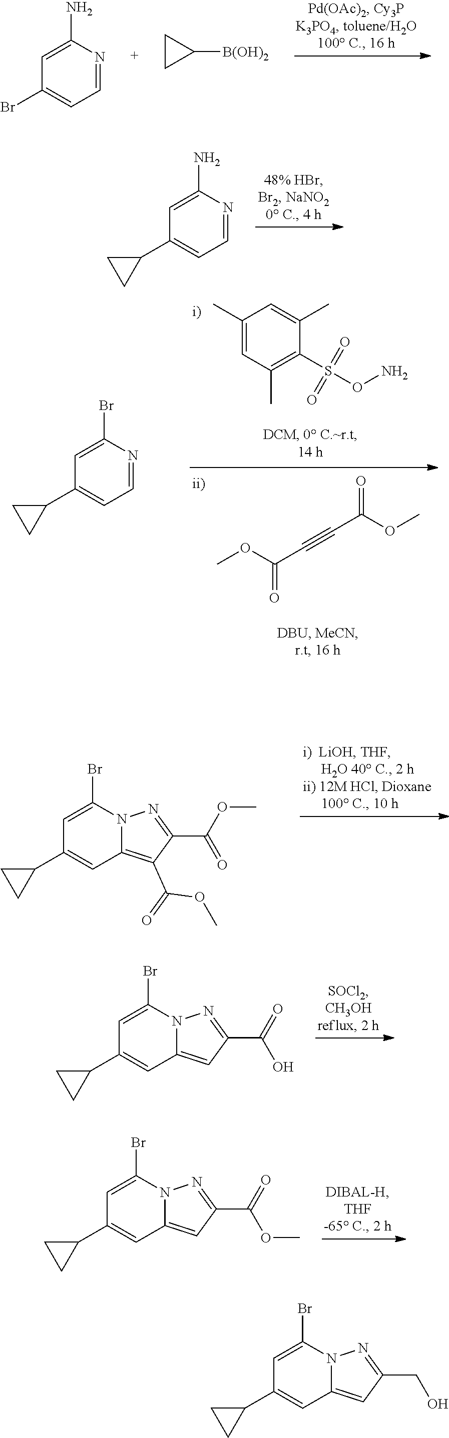

### Synthesis of 4-cyclopropylpyridin-2-amine

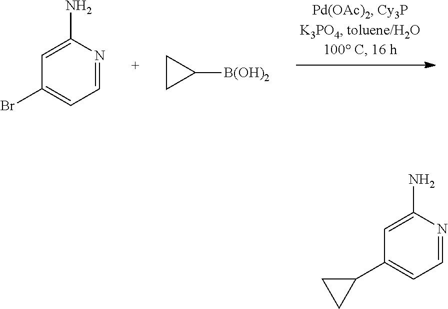 [SMILES: Nc1cc(Br)ccn1 ; OB(O)C1CC1 ; Nc1cc(C2CC2)ccn1]

To a solution of 4-bromopyridin-2-amine (20 g, 0.12 mol) in toluene/H₂O (200 mL/40 mL) was added cyclopropylboronic acid (20 g, 0.23 mol), Pd(OAc)₂ (1.0 g, 4.6 mmol), K₃PO₄ (74 g, 0.35 mol) and PCy₃ (2.6 g, 9.3 mmol) at room temperature. The reaction mixture was stirred at 90° C. for 16 h under nitrogen. The reaction mixture was concentrated in vacuo, diluted with water (150 mL) then extracted with EtOAc (150 ml×2). The combined organic layers were washed with brine (200 mL×1), dried over Na₂SO₄ and concentrated in vacuo give the crude product, which was purified by silica gel chromatography (eluent: EtOAc/PE=1/1) to give 4-cyclopropylpyridin-2-amine (16 g, quant.) as a yellow solid. ESI-MS [M+H]⁺: 135.1.

### Synthesis of (1S,2S)-2-(2-((tert-butoxycarbonyl)amino)-6-methoxypyridin-4-yl)cyclopropane-1-carboxylic acid

To a stirred solution of HBr (40% in H₂O, 70 mL) was added 4-cyclopropylpyridin-2-amine (15 g, 0.11 mol) in portions at 0° C. and then Br₂ (52 g, 0.32 mol) was added dropwise. After stirring at 0° C. for 30 min, a solution of NaNO₂ (19 g, 0.27 mol) in water (30 mL) was added dropwise maintaining the temperature below 10° C. The resulting mixture was stirred for another 30 min at 0° C. The reaction mixture was quenched with NaOH aqueous solution (60 g in water (100 mL)) until pH=12 and extracted with EtOAc (100 mL×2). The combined organic layers were washed with water (200 mL) and brine (200 mL), dried over Na₂SO₄, concentrated in vacuo to give the crude product, which was purified by silica gel chromatography (eluent: EtOAc/PE=1/10) to give 2-bromo-4-cyclopropylpyridine (15 g, 70%) as a yellow oil. ESI-MS [M+H]⁺: 198.1.

### Synthesis of dimethyl 7-bromo-5-cyclopropylpyrazolo[1,5-a]pyridine-2,3-dicarboxylate

To a stirred solution of O-(mesitylsulfonyl)hydroxylamine (34 g, 160 mol) in DCM (200 mL) was added a solution of 2-bromo-4-cyclopropylpyridine (15 g, 76 mmol) in DCM (50 mL) slowly at 0° C. The mixture was stirred at 0° C. for 10 min then warmed to room temperature and stirred for 14 h. The reaction mixture was concentrated and dried in vacuo. To the residue was dissolved in CH₃CN (200 mL) and DMAD (17 g, 120 mmol) was added followed by dropwise addition of DBU (18.2 g, 120 mol) at 0° C. The resulting mixture was stirred at 0° C. for 10 min and warmed to room temperature and stirred for 16 h. The reaction mixture was concentrated in vacuo, diluted with water (300 mL) and extracted with EtOAc (200 mL×2). The combined organic layers were washed with water (400 mL) and brine (400 mL), dried over Na₂SO₄, concentrated in vacuo to give the crude product, which was purified by silica gel chromatography (eluent: EtOAc/PE=1/10) to give dimethyl 7-bromo-5-cyclopropyl pyrazolo[1,5-a]pyridine-2,3-dicarboxylate (6.5 g, 24%) as a yellow solid. ESI-MS [M+H]⁺: 353.0.

### Synthesis of 7-bromo-5-cyclopropylpyrazolo[1,5-a]pyridine-2-carboxylic acid

A mixture of dimethyl 7-bromo-5-cyclopropylpyrazolo[1,5-a]pyridine-2,3-dicarboxylate (6.0 g, 17 mmol) and LiOH·H₂O (1.4 g, 34 mmol) in THF (50 mL) and water (10 mL) was stirred at 40° C. for 2 h. The mixture was concentrated in vacuo to remove then THF then 1,4-dioxane (50 mL) and HCl (conc., 12 mL) were added. The resulting mixture was stirred at 100° C. for 6 h. The reaction mixture was poured into water (200 mL) and extracted with EtOAc (150 mL×3). The combined organic layers were washed with brine (400 mL), dried over Na₂SO₄ then concentrated and dried in vacuo to give 7-bromo-5-cyclopropylpyrazolo[1,5-a]pyridine-2-carboxylic acid (4.8 g, quant.) as a yellow solid. ESI-MS [M+H]⁺: 281.0.

### Synthesis of methyl 7-bromo-5-cyclopropylpyrazolo[1,5-a]pyridine-2-carboxylate

To a mixture of 7-bromo-5-cyclopropylpyrazolo[1,5-a]pyridine-2-carboxylic acid (4.8 g, 17 mmol) in MeOH (50 mL) was added dropwise of SOCl₂ (4.0 g, 34 mmol) at room temperature. The mixture was stirred at 70° C. for 3 h. The reaction mixture was concentrated in vacuo and diluted with EtOAc (200 mL), washed with NaHCO₃ (sat. aq., 150 mL) and brine (150 mL), dried over Na₂SO₄ then concentrated in vacuo to give the crude product. This was purified by silica gel chromatography (eluent: EtOAc/PE=1/10) to give methyl 7-bromo-5-cyclopropylpyrazolo[1,5-a]pyridine-2-carboxylate (3.8 g, 76% for 2 steps) as a yellow solid. ESI-MS [M+H]⁺: 295.0.

### Synthesis of (7-bromo-5-cyclopropylpyrazolo[1,5-a]pyridin-2-yl)methanol

To a stirred solution of methyl 7-bromo-5-cyclopropylpyrazolo[1,5-a]pyridine-2-carboxylate (3.3 g, 11 mmol) in THF (60 mL) was added dropwise DIBAl-H (1 M in hexane, 33 mL, 33 mmol) at −65° C. The mixture was stirred at −65° C. for 2 h. The reaction mixture was quenched with NaOH (1 M in water, 50 mL) at −65° C. and warmed to room temperature, then extracted with EtOAc (100 mL×2). The combined organic layers were washed with brine (150 mL), dried over Na₂SO₄ then concentrated in vacuo to give the crude, which was purified by silica gel chromatography (eluent: EtOAc/PE=1/5) to give the desired product (2.8 g, 95%) as a yellow solid. ESI-MS [M+H]⁺: 267.0.

### Example 1

### Synthesis of N7-((6-cyclopropylimidazo[1,2-a]pyridin-2-yl)methyl)-N4,N4-dimethyl-2-(2-(4-methylpyrimidin-2-yl cyclopropyl)quinoline-4,7-diamine (I-1)

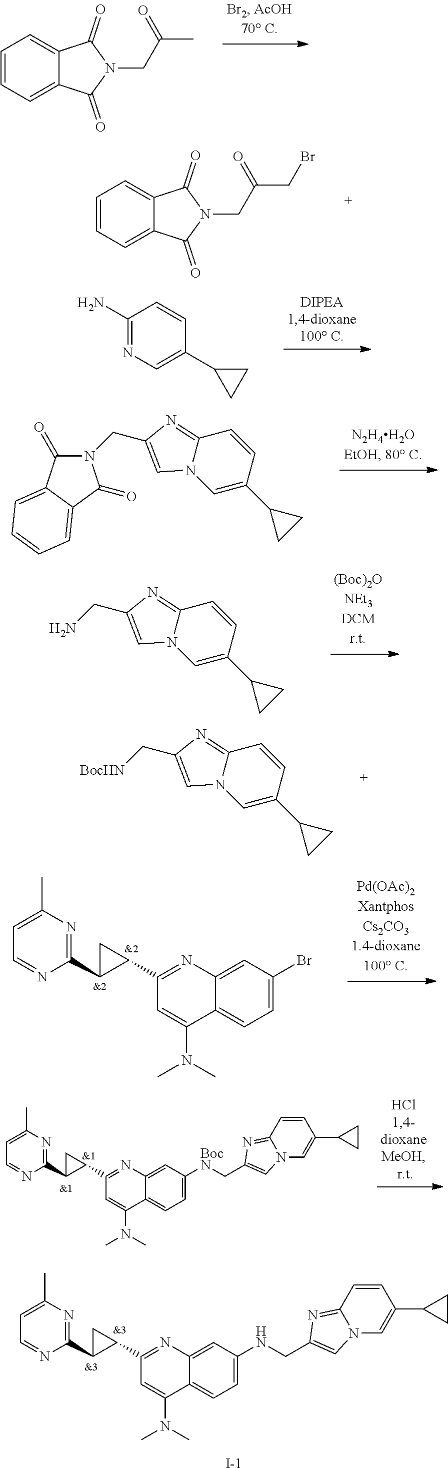

### Synthesis of 2-(3-bromo-2-oxopropyl)isoindoline-1,3-dione

Br₂ (3.8 mL, 74 mmol) in AcOH (45 mL) was added dropwise to a stirred solution of 2-(2-oxopropyl)isoindoline-1,3-dione (10 g, 49 mmol) at 70° C. The mixture was stirred at 70° C. for 90 min then allowed to cool and concentrated in vacuo. The residue was dissolved in DCM (100 mL) and washed with Na₂S₂O₃ (1.0 M aq., 100 mL) and Na₂CO₃ (20% aq., 2×100 mL). The organic layer was passed through a phase separator cartridge then concentrated in vacuo. The residue was triturated with Et₂O to give the title compound (12 g, 88%) as a white solid.

¹H NMR (400 MHz, CDCl₃) δ 7.92-7.88 (m, 2H), 7.78-7.75 (m, 2H), 5.12 (s, 1H), 4.79 (s, 2H), 4.01 (s, 2H).

### Synthesis of 2-((6-cyclopropylimidazo[1,2-a]pyridin-2-yl)methyl)isoindoline-1,3-dione

A mixture of 5-cyclopropylpyridin-2-amine (4.5 g, 34 mmol), 2-(3-bromo-2-oxopropyl)isoindoline-1,3-dione (10 g, 37 mmol) and DIPEA (8.8 mL, 50 mmol) in 1,4-dioxane (340 mL) was heated at 100° C. for 72 h. The mixture was concentrated in vacuo and the residue was purified by silica gel column chromatography, eluting with 20-100% EtOAc in Et₂O to give the title compound (3.6 g, 33%) as a red solid.

¹H NMR (400 MHz, DMSO) δ 8.32 (s, 1H), 7.98-7.91 (m, 4H), 7.79 (s, 1H), 7.40 (d, J=9.3 Hz, 1H), 7.01 (dd, J=1.8, 9.3 Hz, 1H), 4.92 (s, 2H), 2.05-1.92 (m, 1H), 0.96 (ddd, J=4.4, 6.3, 8.4 Hz, 2H), 0.73-0.68 (m, 2H).

### Synthesis of (6-cyclopropylimidazo[1,2-a]pyridin-2-yl)methanamine

N₂H₄·H₂O (3.6 g, 56.72 mmol) was added to a solution of 2-((6-cyclopropylimidazo[1,2-a]pyridin-2-yl)methyl)isoindoline-1,3-dione (3.6 g, 11.34 mmol) in EtOH (55 mL) and the mixture was heated at 80° C. for 18 h. The mixture was cooled, filtered through Celite® and concentrated in vacuo. The residue was loaded onto an SCX cartridge, washed with MeOH in DCM and eluted with (7 N NH₃ in MeOH) in DCM then concentrated in vacuo to give the title compound (2.0 g, 94%) as an orange gum.

¹H NMR (400 MHz, DMSO) δ 8.30 (s, 1H), 7.62 (s, 1H), 7.33 (d, J=9.3 Hz, 1H), 6.92 (dd, J=1.6, 9.2 Hz, 1H), 3.77 (s, 2H), 1.96-1.87 (m, 1H), 0.94-0.87 (m, 2H), 0.69-0.63 (m, 2H).

### Synthesis of tert-butyl ((6-cyclopropylimidazo[1,2-a]pyridin-2-yl)methyl)carbamate

Di-tert-butyl dicarbonate (1.4 g, 5.3 mmol) was added to a solution of (6-cyclopropylimidazo[1,2-a]pyridin-2-yl)methanamine (1.0 g, 5.3 mmol) in DCM (55 mL) and the mixture was stirred at room temperature for 18 h. NaHCO₃ (sat. aq., 50 mL) was added and the mixture was extracted with DCM (3×50 mL). The combined organics were dried over MgSO₄, filtered and concentrated in vacuo. The residue was purified by silica gel column chromatography, eluting with 20-100% EtOAc in cyclohexane to give the title compound (930 mg, 60%) as a yellow solid.

¹H NMR (400 MHz, DMSO) δ 8.32 (s, 1H), 7.57 (s, 1H), 7.35 (d, J=9.3 Hz, 1H), 7.26 (dd, J=5.7, 5.7 Hz, 1H), 6.95 (dd, J=1.9, 9.2 Hz, 1H), 4.19 (d, J=6.1 Hz, 2H), 1.95-1.87 (m, 1H), 1.39 (s, 9H), 0.91 (ddd, J=4.3, 6.3, 8.5 Hz, 2H), 0.69-0.64 (m, 2H).

### Synthesis of rac-tert-butyl ((6-cyclopropylimidazo[1,2-a]pyridin-2-yl)methyl)(4-(dimethylamino)-2-((1S*,2S*)-2-(4-methylpyrimidin-2-yl)cyclopropyl)quinolin-7-yl)carbamate

A mixture of tert-butyl ((6-cyclopropylimidazo[1,2-a]pyridin-2-yl)methyl)carbamate (99 mg, 0.34 mmol), rac-7-bromo-N,N-dimethyl-2-((1S*,2S*)-2-(4-methylpyrimidin-2-yl)cyclopropyl)quinolin-4-amine (110 mg, 0.28 mmol), Pd(OAc)₂, Xantphos (17 mg, 0.028 mmol) and Cs₂CO₃ (190 mg, 0.029 mmol) in 1,4-dioxane (5.0 mL) was degassed with N₂ and heated at 100° C. for 18 h. The mixture was filtered through Celite® and concentrated in vacuo. The residue was purified by silica gel column chromatography, eluting with 0-20% (7 N NH₃ in MeOH) in DCM to give the title compound (50 mg, 30%).

¹H NMR (400 MHz, CDCl₃) δ 8.43 (d, J=5.0 Hz, 1H), 7.89-7.84 (m, 2H), 7.78 (d, J=2.3 Hz, 1H), 7.44-7.40 (m, 3H), 6.93-6.87 (m, 2H), 6.61 (s, 1H), 5.12 (d, J=1.4 Hz, 2H), 2.96 (s, 6H), 2.93-2.87 (m, 2H), 2.80-2.74 (m, 1H), 2.46 (s, 3H), 1.92-1.80 (m, 3H), 1.45 (s, 9H), 0.96-0.91 (m, 2H), 0.69-0.64 (m, 2H).

### Synthesis of rac-N⁷-((6-cyclopropylimidazo[1,2-a]pyridin-2-yl)methyl)-N⁴,N⁴-dimethyl-2-((1S*,2S*)-2-(4-methylpyrimidin-2-yl)cyclopropyl)quinoline-4,7-diamine

HCl (4.0 M in 1,4-dioxane, 0.19 mL, 0.76 mmol) was added to a solution of rac-tert-butyl ((6-cyclopropylimidazo[1,2-a]pyridin-2-yl)methyl)(4-(dimethylamino)-2-((1S*,2S*)-2-(4-methylpyrimidin-2-yl)cyclopropyl)quinolin-7-yl)carbamate (45 mg, 0.076 mmol) in MeOH (1.0 mL) and the mixture was stirred at room temperature for 3 h. The mixture was concentrated in vacuo and purified by preparative HPLC to give the title compound (3 mg, 8%) as a formic acid salt.

ESI-MS (M+H)+: 490.5, ¹H NMR (400 MHz, DMSO) δ 8.50 (d, J=5.1 Hz, 1H), 8.31 (s, 2H), 7.70-7.67 (m, 2H), 7.40 (d, J=9.3 Hz, 1H), 7.16 (d, J=5.1 Hz, 1H), 6.99-6.91 (m, 2H), 6.70 (d, J=2.4 Hz, 1H), 6.62 (dd, J=5.7, 5.7 Hz, 1H), 6.59 (s, 1H), 4.44 (d, J=5.5 Hz, 2H), 2.88 (s, 6H), 2.75-2.65 (m, 2H), 2.61-2.55 (m, 2H), 2.40 (s, 3H), 1.94-1.86 (m, 1H), 1.77-1.63 (m, 2H), 1.23 (s, 1H), 0.92-0.87 (m, 2H), 0.68-0.63 (m, 2H).

### Example 2

### Synthesis of 2-((1S,2S)-2-(3-chlorophenyl)cyclopropyl)-7-(((6-cyclopropylimidazo[1,2-a]pyridin-2-yl)methyl)amino)pyrido[4,3-d]pyrimidin-4 (3H)-one (I-2)

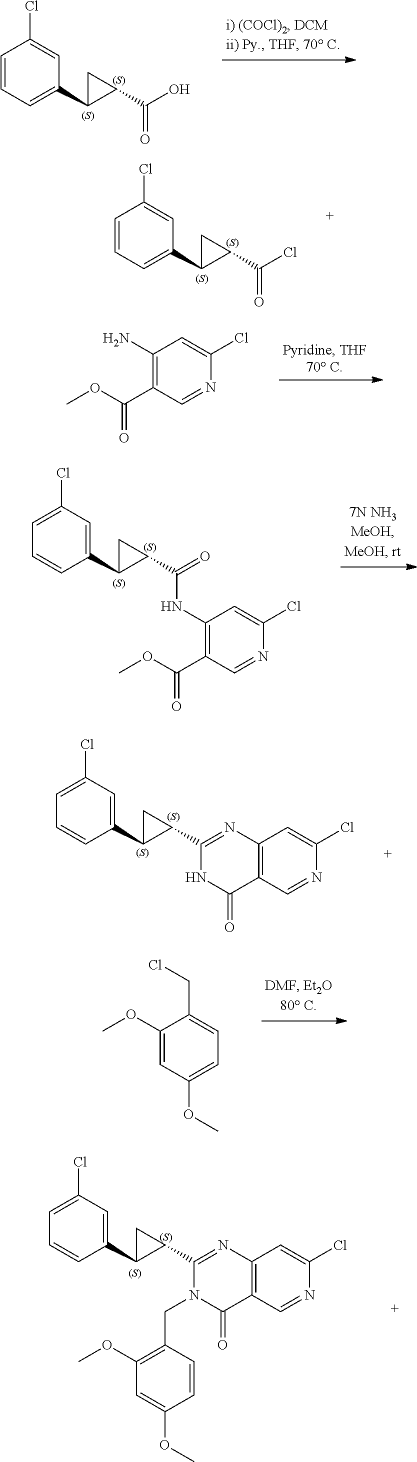

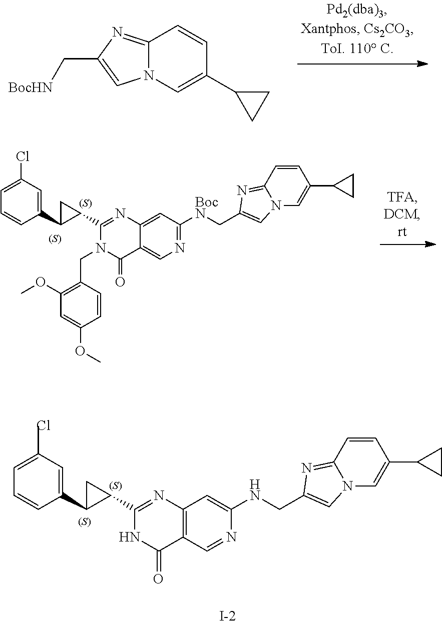

### Synthesis of methyl 6-chloro-4-((1S,2S)-2-(3-chlorophenyl)cyclopropane-1-carboxamido) nicotinate

To a cooled solution of (1S,2S)-2-(3-chlorophenyl)cyclopropane-1-carboxylic acid (0.45 g, 2.3 mmol), DCM (10 mL), at 0° C. was added one drop of DMF and the mixture was stirred for 2 h. The mixture was concentrated in vacuo and added to a solution of methyl 4-amino-6-chloronicotinate (0.36 g, 1.92 mmol) in a mixture of THF (10 mL), pyridine (1.6 mL, 19.2 mmol) and heated to 70° C. for 18 h. The mixture was diluted with NaHCO₃ (sat. aq., 50 mL) and extracted with EtOAc (3×50 mL). The organic phases were combined, dried over MgSO₄ and concentrated in vacuo. The residue was purified by silica gel column chromatography, eluting with a gradient of 0-100% EtOAc in cyclohexane to give the title compound (335 mg, 48%).

ESI-MS (M+H)+: 365.1, ¹H NMR (400 MHz, CDCl₃) δ 11.45 (s, 1H), 8.94 (s, 1H), 8.73 (s, 1H), 7.24-7.21 (m, 2H), 7.10 (d, J=1.9 Hz, 1H), 7.05-6.99 (m, 1H), 3.97 (s, 3H), 2.62 (ddd, J=4.0, 6.5, 9.3 Hz, 1H), 1.91-1.86 (m, 1H), 1.79-1.74 (m, 1H), 1.49-1.44 (m, 1H).

### Synthesis of 7-chloro-2-((1S,2S)-2-(3-chlorophenyl)cyclopropyl)pyrido[4,3-d]pyrimidin-4 (3H)-one

Methyl 6-chloro-4-((1S,2S)-2-(3-chlorophenyl)cyclopropane-1-carboxamido)nicotinate was added to a mixture of 7 N NH₃ in MeOH (13 mL) and MeOH (13 mL) in a sealed vial. The mixture was stirred at room temperature for 18 h. The mixture was concentrated in vacuo and the residues was purified by silica gel column chromatography, eluting with a gradient of 20-100% EtOAc in cyclohexane to give the title compound (179 mg, 58%).

ESI-MS (M+H)+: 332.1, ¹H NMR (400 MHz, DMSO) δ 12.88 (s, 1H), 9.01 (s, 1H), 7.56 (s, 1H), 7.37-7.32 (m, 2H), 7.30-7.27 (m, 1H), 7.22-7.20 (m, 1H), 2.72-2.66 (m, 1H), 2.33-2.27 (m, 1H), 1.90-1.84 (m, 1H), 1.75 (ddd, J=4.5, 6.6, 8.4 Hz, 1H).

### Synthesis of 7-chloro-2-((1S,2S)-2-(3-chlorophenyl)cyclopropyl)-3-(2,4-dimethoxybenzyl)pyrido [4,3-d]pyrimidin-4 (3H)-one

A mixture of K₂CO₃ (0.15 g, 1.08 mmol), 7-chloro-2-((1S,2S)-2-(3-chlorophenyl)cyclopropyl)pyrido[4,3-d]pyrimidin-4 (3H)-one (0.18 g, 0.542 mmol), DMB-Cl (0.13 mL, 0.813 mmol) in a mixture of DMF (5 mL) and Et₂O (2 mL) was heated to 80° C. for 18 h. Mixture was diluted with brine (50 mL), water (75 mL) and extracted with EtOAc (3×25 mL). The organic phases were combined, concentrated in vacuo, the residue was purified by silica gel column chromatography, eluting with a gradient of 0-100% EtOAc in cyclohexane to give the title compound (111 mg, 43%).

ESI-MS (M+H)+: 482.1, ¹H NMR (400 MHz, DMSO) δ 9.11 (s, 1H), 7.63 (s, 1H), 7.25-7.23 (m, 2H), 7.03 (s, 1H), 6.99-6.96 (m, 1H), 6.77 (d, J=8.5 Hz, 1H), 6.38 (d, J=2.4 Hz, 1H), 6.33 (dd, J=2.4, 8.4 Hz, 1H), 5.35-5.21 (m, 2H), 3.68 (s, 3H), 3.62 (s, 3H), 2.45 (ddd, J=4.1, 6.5, 9.0 Hz, 1H), 2.36-2.31 (m, 1H), 2.01-1.94 (m, 1H), 1.61 (ddd, J=4.4, 6.6, 8.1 Hz, 1H).

### Synthesis of tert-butyl (2-((1S,2S)-2-(3-chlorophenyl)cyclopropyl)-3-(2,4-dimethoxybenzyl)-4-oxo-3,4-dihydropyrido[4,3-d]pyrimidin-7-yl)((6-cyclopropylimidazo[1,2-a]pyridin-2-yl)methyl)carbamate

A mixture containing 7-chloro-2-((1S,2S)-2-(3-chlorophenyl)cyclopropyl)-3-(2,4-dimethoxybenzyl)pyrido [4,3-d]pyrimidin-4 (3H)-one (0.11 g, 0.22 mmol), tert-butyl ((6-cyclopropylimidazo[1,2-a]pyridin-2-yl)methyl)carbamate (0.079 g, 0.22 mmol), Pd₂(dba)₃ (0.021 g, 0.02 mmol), Xantphos (0.026 g, 0.045 mmol), Cs₂CO₃ (0.15 g, 0.45 mmol) in toluene (2 mL) was heated to 110° C. for 18 h. The mixture was filtered through Celite® and concentrated in vacuo. The residue was purified by silica gel column chromatography, eluting with a gradient of 0-100% EtOAc in cyclohexane to give the title compound (64 mg, 38%) as a pale yellow gum.

ESI-MS (M+H)+: 733.5, ¹H NMR (400 MHz, DMSO) δ 9.08 (s, 1H), 8.27 (s, 1H), 7.80 (s, 1H), 7.58 (d, J=4.4 Hz, 1H), 7.37-7.32 (m, 1H), 7.25-7.22 (m, 2H), 7.03 (s, 1H), 6.99-6.91 (m, 2H), 6.72 (d, J=8.4 Hz, 1H), 6.38 (d, J=2.3 Hz, 1H), 6.34 (dd, J=2.3, 8.4 Hz, 1H), 5.34 (s, 2H), 5.32-5.20 (m, 2H), 3.68 (s, 3H), 3.63 (s, 3H), 2.47-2.40 (m, 1H), 2.33-2.27 (m, 1H), 1.99-1.87 (m, 2H), 1.62-1.54 (m, 1H), 1.45 (s, 9H), 0.93-0.86 (m, 2H), 0.68-0.62 (m, 2H).

### Synthesis of 2-((1S,2S)-2-(3-chlorophenyl)cyclopropyl)-7-(((6-cyclopropylimidazo[1,2-a]pyridin-2-yl)methyl)amino)pyrido[4,3-d]pyrimidin-4 (3H)-one

TFA (0.5 mL) was added to a solution of tert-butyl (2-((1S,2S)-2-(3-chlorophenyl)cyclopropyl)-3-(2,4-dimethoxybenzyl)-4-oxo-3,4-dihydropyrido[4,3-d]pyrimidin-7-yl)((6-cyclopropylimidazo[1,2-a]pyridin-2-yl)methyl)carbamate (50 mg, 0.068 mmol) and anisole (0.074 mL, 0.68 mmol) in DCM (0.5 mL). The mixture was stirred at room temperature for 2 h. Mixture was concentrated in vacuo onto silica and purified by silica gel column chromatography, eluting with a gradient of 0-10% (7 N NH₃ in MeOH) in DCM followed by preparative HPLC to give the title compound (11 mg, 33%) as a formic acid salt.

ESI-MS (M+H)+: 483.5, ¹H NMR (400 MHz, DMSO) δ 12.02 (s, 1H), 8.73 (s, 1H), 8.39 (s, 1H), 8.30 (s, 1H), 7.68 (dd, J=6.0, 6.0 Hz, 1H), 7.64 (s, 1H), 7.38 (d, J=9.3 Hz, 1H), 7.34-7.29 (m, 2H), 7.27-7.25 (m, 1H), 7.18 (d, J=7.7 Hz, 1H), 6.96 (dd, J=1.8, 9.3 Hz, 1H), 6.31 (s, 1H), 4.58 (d, J=3.8 Hz, 2H), 2.62-2.54 (m, 1H), 2.22-2.16 (m, 1H), 1.94-1.86 (m, 1H), 1.80-1.74 (m, 1H), 1.63-1.57 (m, 1H), 0.93-0.88 (m, 2H), 0.68-0.63 (m, 2H).

### Example 3

### Synthesis of rac-2-((1S*,2S*)-2-(3-chlorophenyl)cyclopropyl)-7-(((6-cyclopropylimidazo[1,2-a]pyridin-2-yl)methyl)amino)pyrido[4,3-d]pyrimidin-4(3H)-one (I-3)

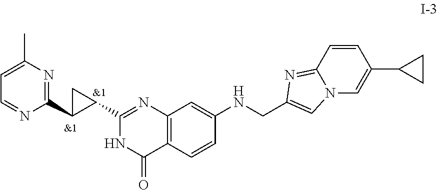 [SMILES: Cc1ccnc([C@H]2C[C@@H]2c2nc3cc(NCc4cn5cc(C6CC6)ccc5n4)ccc3c(=O)[nH]2)n1]

The title compound was synthesised in a similar manner as described for 2-((1S,2S)-2-(3-chlorophenyl)cyclopropyl)-7-(((6-cyclopropylimidazo[1,2-a]pyridin-2-yl)methyl)amino)pyrido[4,3-d]pyrimidin-4 (3H)-one starting from methyl 2-amino-4-bromobenzoate and rac-(1S*,2S*)-2-(4-methylpyrimidin-2-yl)cyclopropane-1-carbonyl chloride (2.3 mg, 25%).

ESI-MS (M+H)+: 464.3, ¹H NMR (400 MHz, DMSO) δ 11.87 (s, 1H), 8.53 (d, J=5.0 Hz, 1H), 8.31 (s, 1H), 7.72 (d, J=8.8 Hz, 1H), 7.67 (s, 1H), 7.39 (d, J=9.4 Hz, 1H), 7.20 (d, J=5.0 Hz, 1H), 7.10 (dd, J=5.8, 5.8 Hz, 1H), 6.98 (d, J=9.4 Hz, 1H), 6.79 (dd, J=2.3, 8.8 Hz, 1H), 6.51 (d, J=1.9 Hz, 1H), 4.44 (d, J=5.6 Hz, 2H), 2.77-2.71 (m, 1H), 2.48-2.45 (m, 1H), 2.41 (s, 3H), 1.94-1.86 (m, 1H), 1.74-1.61 (m, 2H), 0.93-0.87 (m, 2H), 0.69-0.63 (m, 2H).

### Example 4

### Synthesis of rac-1-(2-(((2-((7S*,2S*)-2-(3-chlorophenyl)cyclopropyl)-4-methoxyquinolin-7-yl)amino)methyl)-6-cyclopropylimidazo[1,2-a]pyridin-8-yl)pyrrolidin-2-one (I-4)

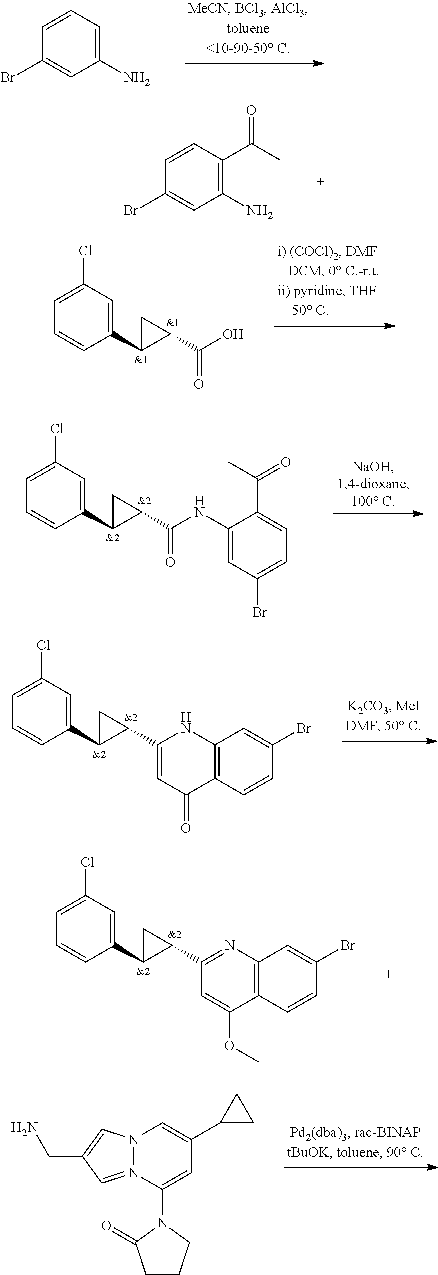

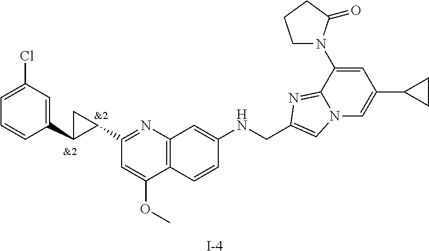

### Synthesis of 1-(2-(aminomethyl)-6-cyclopropylimidazo[1,2-a]pyridin-8-yl)pyrrolidin-2-one

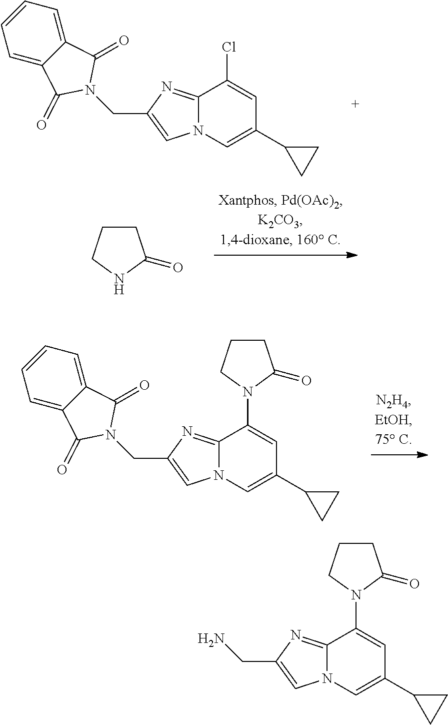

### Synthesis of 2-((6-cyclopropyl-8-(2-oxopyrrolidin-1-yl)imidazo[1,2-a]pyridin-2-yl)methyl)isoindoline-1,3-dione

Pyrrolidine-2-one (0.65 mL, 8.5 mmol), 2-((8-chloro-6-cyclopropylimidazo[1,2-a]pyridin-2-yl)methyl)isoindoline-1,3-dione (2000 mg, 5.7 mmol), Xantphos (660 mg, 1.1 mmol) and K₂CO₃ (1600 mg, 11 mmol) in 1,4-dioxane (20 mL) was degassed for 5 min with N₂. Pd(OAc)₂ (130 mg, 0.57 mmol) was added and the reaction mixture was heated in a microwave at 160° C. for 2 h. The mixture was cooled to room temperature, filtered through Celite® and concentrated in vacuo. The residue was purified by silica gel chromatography eluting with a gradient of 20-100% EtOAc in cyclohexane to give the title compound (1.0 g, 44%).

ESI-MS (M+H)+: 401.3, ¹H NMR (400 MHz, CDCl₃) δ 7.87 (dd, J=3.0, 5.5 Hz, 2H), 7.72 (dd, J=3.1, 5.5 Hz, 2H), 7.69-7.68 (m, 1H), 7.45 (s, 1H), 7.24 (d, J=1.5 Hz, 1H), 5.02 (s, 2H), 4.27 (dd, J=7.1, 7.1 Hz, 2H), 2.58 (t, J=8.2 Hz, 2H), 2.22-2.13 (m, 2H), 1.90-1.82 (m, 1H), 0.95-0.89 (m, 2H), 0.68-0.62 (m, 2H).

### Synthesis of 1-(2-(aminomethyl)-6-cyclopropylimidazo[1,2-a]pyridin-8-yl)pyrrolidin-2-one

A solution of 2-((6-cyclopropyl-8-(2-oxopyrrolidin-1-yl)imidazo[1,2-a]pyridin-2-yl)methyl)isoindoline-1,3-dione (1.0 g, 2.50 mmol) and hydrazine hydrate (0.16 mL, 4.99 mmol) in EtOH (15 mL) was heated to 75° C. for 2 h. The mixture was cooled to room temperature then applied to an SCX column which was washed with 3:1 DCM:MeOH then eluted with 3:1 (7 N NH₃ in MeOH):DCM to give the title compound (0.60 g, 89%).

ESI-MS (M+H)+: 271.3, ¹H NMR (400 MHz, CDCl₃) δ 7.77 (s, 1H), 7.40 (s, 1H), 7.19 (d, J=1.5 Hz, 1H), 4.28 (t, J=7.2 Hz, 2H), 3.99 (d, J=0.6 Hz, 2H), 2.62 (t, J=8.2 Hz, 2H), 2.28-2.20 (m, 2H), 1.93-1.85 (m, 1H), 0.97-0.91 (m, 2H), 0.72-0.66 (m, 2H). NH₂ not observed

### Synthesis of 1-(2-amino-4-bromophenyl)ethan-1-one

BCl₃ (1.0 M in DCM, 32 mL, 32 mmol) was added dropwise to a stirred slurry of 3-bromoaniline (5.0 g, 29 mmol) in MeCN (15 mL, 290 mmol) and toluene (35 mL) while the mixture temperature was maintained below 10° C. AlCl₃ (5.8 g, 44 mmol) was added portion-wise to the reaction mixture with cooling. The mixture was stirred at 90° C. for 17 h. The reaction mixture was cooled to room temperature and HCl (2.0 M aq., 50 mL) was added. The reaction mixture was stirred at 50° C. for 1 h. The reaction mixture was cooled to room temperature, diluted with water (100 mL) and extracted with DCM (3×150 mL). The combined organics were dried over MgSO₄ and concentrated in vacuo to give the title compound (4.6 g, 74%) as a brown solid.

¹H NMR (400 MHz, CDCl₃) δ 7.55 (d, J=8.6 Hz, 1H), 6.83 (d, J=1.8 Hz, 1H), 6.76 (dd, J=2.0, 8.6 Hz, 1H), 6.38-6.33 (m, 2H), 2.54 (s, 3H).

### Synthesis of rac-(1S*,2S*)—N-(2-acetyl-5-bromophenyl)-2-(3-chlorophenyl)cyclopropane-1-carboxamide

Oxalyl chloride (0.44 g, 5.1 mmol) was added dropwise to a solution of rac-(1S*,2S*)-2-(3-chlorophenyl)cyclopropane-1-carboxylic acid (500 mg, 2.5 mmol) and DMF (0.05 mL) in DCM (10 mL) to 0° C. The reaction mixture was stirred at 0° C. for 2.5 h. The reaction mixture was allowed to warm to room temperature and concentrated in vacuo. The residue was suspended in THF (10 mL) and pyridine (2.1 mL, 25 mmol) and 1-(2-amino-4-bromophenyl)ethan-1-one (520 mg, 2.4 mmol) were added. The reaction mixture was stirred at 50° C. for 16 h. The reaction mixture was cooled to room temperature, diluted with water (75 mL) and stirred for 2 h. The resulting precipitate was collected by filtration, washed with water (100 mL) and dried in vacuo to give the title compound (850 mg, 85%).

¹H NMR (400 MHz, DMSO) δ 11.51 (s, 1H), 8.57 (d, J=2.0 Hz, 1H), 7.92 (d, J=8.6 Hz, 1H), 7.42 (dd, J=2.0, 8.3 Hz, 1H), 7.35-7.29 (m, 2H), 7.28-7.24 (m, 1H), 7.21-7.18 (m, 1H), 2.59 (s, 3H), 2.48-2.43 (m, 1H), 2.21-2.15 (m, 1H), 1.56-1.42 (m, 2H).

### Synthesis of rac-7-bromo-2-((1S*,2S*)-2-(3-chlorophenyl)cyclopropyl)quinolin-4(H)-one

NaOH (100 mg, 2.6 mmol) was azeotroped with toluene (2×) and added to a solution of rac-(1S*,2S*)—N-(2-acetyl-5-bromophenyl)-2-(3-chlorophenyl)cyclopropane-1-carboxamide (250 mg, 0.64 mmol) in 1,4-dioxane (5.0 mL) which was placed under a N₂ atmosphere. The reaction mixture was stirred at 100° C. for 3 h then cooled to room temperature and diluted with NH₄Cl (sat. aq., 20 mL). The resulting precipitate was collected by filtration, washed with water (50 mL) and dried in vacuo to give the title compound (200 mg, 83%) as a beige solid.

¹H NMR (400 MHz, DMSO) δ 11.65-11.62 (m, 1H), 7.94 (d, J=8.6 Hz, 1H), 7.75 (d, J=1.3 Hz, 1H), 7.42 (dd, J=1.5, 8.6 Hz, 1H), 7.37-7.31 (m, 2H), 7.27 (dd, J=1.8, 6.8 Hz, 1H), 7.22 (d, J=7.8 Hz, 1H), 5.89 (s, 1H), 2.27-2.20 (m, 1H), 1.80-1.72 (m, 1H), 1.69-1.61 (m, 1H), one CH hidden under DMSO peak.

### Synthesis of rac-7-bromo-2-((1S*,2S*)-2-(3-chlorophenyl)cyclopropyl)-4-methoxyquinoline

A mixture of rac-7-bromo-2-((1S*,2S*)-2-(3-chlorophenyl)cyclopropyl)quinolin-4 (1H)-one (100 mg, 0.27 mmol) and K₂CO₃ (74 mg, 0.53 mmol) in DMF (2.5 mL) was stirred at 50° C. for 1.5 h. The reaction mixture was cooled to room temperature and MeI (0.019 mL, 0.29 mmol) was added. The reaction mixture was stirred at 50° C. for 5 h then cooled to room temperature and further MeI (0.019 mL, 0.29 mmol) was added. The reaction mixture was stirred at 50° C. for 16 h. The reaction mixture was cooled to room temperature, quenched with water (25 mL) and extracted with EtOAc (3×40 mL). The combined organics were dried over MgSO₄ and concentrated in vacuo to give the title compound (82 mg, 79%) as a yellow oil.

¹H NMR (400 MHz, CDCl₃) δ 8.09 (d, J=1.8 Hz, 1H), 7.96 (d, J=8.8 Hz, 1H), 7.48 (dd, J=1.9, 8.8 Hz, 1H), 7.24-7.14 (m, 3H), 7.07 (d, J=7.4 Hz, 1H), 6.67 (s, 1H), 4.02 (s, 3H), 2.67-2.61 (m, 1H), 2.38-2.33 (m, 1H), 2.01-1.96 (m, 1H), 1.54-1.49 (m, 1H).

### Synthesis of rac-1-(2-(((2-((7S*,2S*)-2-(3-chlorophenyl)cyclopropyl)-4-methoxyquinolin-7-yl)amino)methyl)-6-cyclopropylimidazo[1,2-a]pyridin-8-yl)pyrrolidin-2-one

A mixture of rac-7-bromo-2-((1S*,2S*)-2-(3-chlorophenyl)cyclopropyl)-4-methoxyquinoline (41 mg, 0.11 mmol), 1-(2-(aminomethyl)-6-cyclopropylimidazo[1,2-a]pyridin-8-yl)pyrrolidin-2-one (34 mg, 0.13 mmol), rac-BINAP (6.6 mg, 0.010 mmol), Pd₂(dba)₃ (4.8 mg, 0.0053 mmol) and tBuOK (24 mg, 0.21 mmol) in toluene (1.5 mL) was degassed for 5 min and stirred at 90° C. for 4 h. The reaction mixture was cooled to room temperature and filtered through Celite® and concentrated in vacuo. The residue was dissolved in DCM (25 mL) and washed with NH₄Cl (sat. aq., 3×50 mL). The combined organics were passed through a hydrophobic frit and concentrated in vacuo. The residue was purified by silica gel column chromatography, eluting with 5% MeOH in DCM then 10% (7N NH₃ in MeOH) in DCM to give the title compound (25 mg, 41%) as a yellow solid.

ESI-MS (M+H)+: 578.5, ¹H NMR (400 MHz, DMSO) δ 8.24 (s, 1H), 7.75-7.69 (m, 2H), 7.32-7.15 (m, 5H), 7.13 (d, J=1.6 Hz, 1H), 6.95 (dd, J=2.2, 9.0 Hz, 1H), 6.75-6.70 (m, 2H), 4.45 (d, J=5.6 Hz, 2H), 4.19 (dd, J=7.3, 7.3 Hz, 2H), 3.94 (s, 3H), 2.48-2.42 (m, 2H), 2.18-2.09 (m, 2H), 1.94-1.86 (m, 1H), 1.82-1.77 (m, 1H), 1.54-1.43 (m, 1H), 0.93-0.88 (m, 2H), 0.66-0.61 (m, 2H) 2 CH hidden under solvent peaks.

### Example 5

### Synthesis of rac-1-(2-(((2-((1S*,2S*)-2-(3-chlorophenyl)cyclopropyl)-4-oxo-1,4-dihydroquinolin-7-yl)amino)methyl)-6-cyclopropylimidazo[1,2-a]pyridin-8-yl)-3-methylimidazolidine-2,4-dione (I-5)

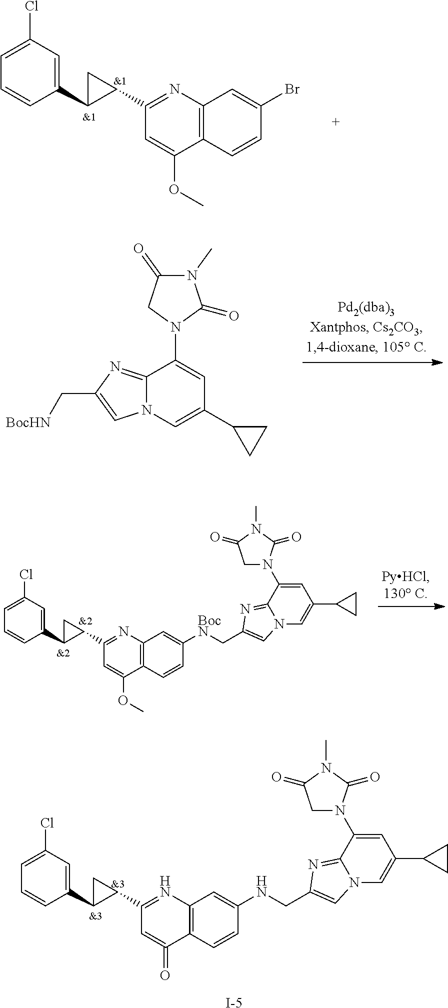

### Synthesis of rac-tert-butyl (2-((1S*,2S*)-2-(3-chlorophenyl)cyclopropyl)-4-methoxyquinolin-7-yl)((6-cyclopropyl-8-(3-methyl-2,4-dioxoimidazolidin-1-yl)imidazo[1,2-a]pyridin-2-yl)methyl)carbamate

rac-7-bromo-2-((1S*,2S*)-2-(3-chlorophenyl)cyclopropyl)-4-methoxyquinoline (100 mg, 0.26 mmol) was added to a mixture of tert-butyl ((6-cyclopropyl-8-(3-methyl-2,4-dioxoimidazolidin-1-yl)imidazo[1,2-a]pyridin-2-yl)methyl)carbamate (123 mg, 0.31 mmol), Pd₂(dba)₃ (12 mg, 0.013 mmol), Xantphos (15 mg, 0.026 mmol), Cs₂CO₃ (251 mg, 0.77 mmol) in 1,4-dioxane (5.0 mL) and the mixture was heated to 105° C. for 18 h. Mixture was filtered through Celite® then concentrated in vacuo. The residue was purified by silica gel column chromatography, eluting with a gradient of 0-100% EtOAc in cyclohexane to give crude title compound which was taken on to the next stage without further purification.

¹H NMR (400 MHz, CDCl₃) δ 8.01 (d, J=9.0 Hz, 1H), 7.81 (d, J=1.9 Hz, 1H), 7.75 (d, J=0.4 Hz, 1H), 7.48 (s, 1H), 7.37 (d, J=1.8 Hz, 1H), 7.23-7.14 (m, 3H), 7.11-7.05 (m, 2H), 6.60 (s, 1H), 5.05 (s, 2H), 4.02-4.01 (m, 3H), 3.70 (s, 2H), 3.11 (s, 3H), 2.62-2.58 (m, 1H), 2.38-2.33 (m, 1H), 1.97-1.83 (m, 2H), 1.51-1.47 (m, 1H), 1.45 (s, 9H), 0.98-0.88 (m, 2H), 0.71-0.64 (m, 2H).

### Synthesis of rac-1-(2-(((2-((1S*,2S*)-2-(3-chlorophenyl)cyclopropyl)-4-oxo-1,4-dihydroquinolin-7-yl)amino)methyl)-6-cyclopropylimidazo[1,2-a]pyridin-8-yl)-3-methylimidazolidine-2,4-dione

Pyridine HCl (392 mg, 3.39 mmol) was added to rac-tert-butyl (2-((1S*,2S*)-2-(3-chlorophenyl)cyclopropyl)-4-methoxyquinolin-7-yl)((6-cyclopropyl-8-(3-methyl-2,4-dioxoimidazolidin-1-yl)imidazo[1,2-a]pyridin-2-yl)methyl)carbamate (60 mg, 0.085 mmol) and the mixture was heated to 130° C. for 2 h. The mixture was diluted with water and 10% MeOH in DCM then extracted with 10% MeOH in DCM. The organic phases were combined, dried then concentrated in vacuo. The residue was purified by preparative HPLC to give the title compound (3 mg, 3%).

ESI-MS (M+H)+: 593.3, Purity: 91.4%, ¹H NMR (400 MHz, DMSO) δ 10.98-10.97 (m, 1H), 8.26 (d, J=1.1 Hz, 1H), 7.74-7.70 (m, 2H), 7.35-7.29 (m, 3H), 7.27-7.25 (m, 1H), 7.20-7.18 (m, 1H), 6.97-6.95 (m, 1H), 6.70 (dd, J=2.1, 8.8 Hz, 1H), 6.45 (d, J=2.0 Hz, 1H), 5.60 (d, J=1.7 Hz, 1H), 4.91 (s, 2H), 4.43 (d, J=5.4 Hz, 2H), 2.99 (s, 3H), 2.40-2.36 (m, 1H), 2.16-2.12 (m, 1H), 1.94 (dd, J=8.4, 8.4 Hz, 1H), 1.68-1.65 (m, 1H), 1.54-1.51 (m, 1H), 0.97-0.92 (m, 2H), 0.67-0.62 (m, 2H).

### Example 6

### Synthesis of 1-(2-(((2-((1S,2S)-2-(3-chlorophenyl)cyclopropyl)-4-oxo-3,4-dihydroquinazolin-7-yl)amino)methyl)-6-cyclopropylimidazo[1,2-a]pyridin-8-yl)-3-methylimidazolidine-2,4-dione (I-6)

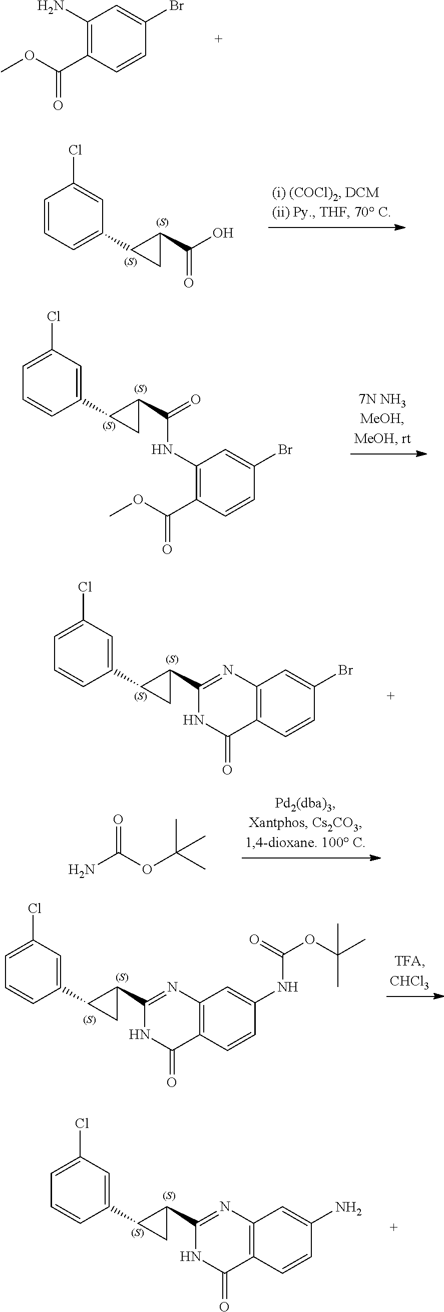

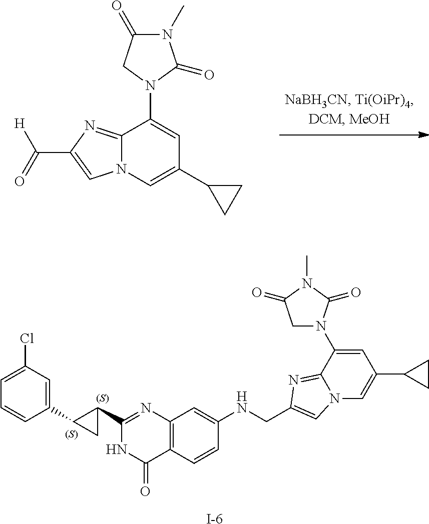

### Synthesis of methyl 4-bromo-2-((1S,2S)-2-(3-chlorophenyl)cyclopropane-1-carboxamido)benzoate

To a cooled solution of (1S,2S)-2-(3-chlorophenyl)cyclopropane-1-carboxylic acid (1.0 g, 5.1 mmol), and oxalyl chloride (0.44 mL, 5.1 mmol) in DCM (20 mL), at 0° C., was added one drop of DMF and the mixture was stirred for 1 h. The mixture was concentrated then re-dissolved in THF (20 mL). Methyl 2-amino-4-bromobenzoate (1.2 g, 5.1 mmol) then pyridine (1.2 mL, 15.3 mmol) were added and the mixture was heated to 70° C. for 18 h. The cooled mixture was diluted with water then extracted with DCM. The combined organics were passed through a hydrophobic cartridge, concentrated in vacuo onto silica then purified by silica gel column chromatography, eluting with a gradient of 0-100% EtOAc in cyclohexane to give the title compound (800 mg, 38%).

ESI-MS (M+H)+: 410.1, ¹H NMR (400 MHz, DMSO) δ 10.88 (s, 1H), 8.50 (d, J=2.0 Hz, 1H), 7.83 (d, J=8.5 Hz, 1H), 7.41 (dd, J=2.0, 8.5 Hz, 1H), 7.35-7.30 (m, 2H), 7.28-7.25 (m, 1H), 7.20 (d, J=7.7 Hz, 1H), 3.84 (s, 3H), 2.48-2.43 (m, 1H), 2.25-2.19 (m, 1H), 1.56-1.43 (m, 2H).

### Synthesis of 7-bromo-2-((1S,2S)-2-(3-chlorophenyl)cyclopropyl)quinazolin-4 (3H)-one

7N NH₃ in MeOH (20 mL) was added to a solution of methyl 4-bromo-2-((1S,2S)-2-(3-chlorophenyl)cyclopropane-1-carboxamido)benzoate (400 mg, 0.98 mmol) in MeOH (20 mL) and the mixture was heated to 80° C. for 18 h. The mixture was diluted with DCM, washed with brine, passed through a hydrophobic cartridge and concentrated on to silica in vacuo then purified by silica gel column chromatography, eluting with a gradient of 0-80% EtOAc in cyclohexane to give the title compound which was used directly in the next step.

¹H NMR (400 MHz, DMSO) δ 12.55 (s, 1H), 7.97 (d, J=8.4 Hz, 1H), 7.73 (s, 1H), 7.59-7.56 (m, 1H), 7.36-7.30 (m, 2H), 7.28 (d, J=7.8 Hz, 1H), 7.21 (d, J=7.5 Hz, 1H), 2.65-2.59 (m, 1H), 2.33-2.25 (m, 1H), 1.91-1.80 (m, 1H), 1.68-1.63 (m, 1H).

### Synthesis of tert-butyl (2-((1S,2S)-2-(3-chlorophenyl)cyclopropyl)-4-oxo-3,4-dihydroquinazolin-7-yl)carbamate

A solution of methyl 4-bromo-2-((1S,2S)-2-(3-chlorophenyl)cyclopropane-1-carboxamido) benzoate (100 mg, 0.27 mmol), Xantphos (62 mg, 0.11 mmol), tert-butyl carbamate (34 mg, 0.29 mmol), Cs₂CO₃ (260 mg, 0.8-0 mmol) in 1,4-dioxane was purged with N₂ for 5 min and heated to 100° C. for 18 h. The mixture was filtered through Celite®, concentrated in vacuo, then purified by silica gel column chromatography, eluting with a gradient of 0-80% EtOAc in cyclohexane to give the title compound (120 mg, quant).

¹H NMR (400 MHz, CDCl₃) δ 10.97 (s, 1H), 8.12 (d, J=8.8 Hz, 1H), 7.68 (d, J=2.0 Hz, 1H), 7.38 (dd, J=2.1, 8.8 Hz, 1H), 7.24-7.17 (m, 3H), 7.11-7.08 (m, 1H), 6.76 (s, 1H), 2.77-2.70 (m, 1H), 2.14-2.08 (m, 1H), 2.00-1.93 (m, 1H), 1.56-1.50 (m, 10H).

### Synthesis of 7-amino-2-((1S,2S)-2-(3-chlorophenyl)cyclopropyl)quinazolin-4 (3H)-one

TFA (2.0 mL) was added to a mixture of tert-butyl (2-((1S,2S)-2-(3-chlorophenyl)cyclopropyl)-4-oxo-3,4-dihydroquinazolin-7-yl)carbamate (200 mg, 0.48 mmol) in CHCl₃ (2 mL). The reaction was heated to 70° C. for 30 min, under microwave conditions. The mixture was treated with NaHCO₃ (sat. aq.) then extracted with DCM. The combined organics were passed through a hydrophobic cartridge then concentrated in vacuo to give the title compound (124 mg, 82%).

ESI-MS (M+H)+: 312.3

### Synthesis of 1-(2-(((2-((1S,2S)-2-(3-chlorophenyl)cyclopropyl)-4-oxo-3,4-dihydroquinazolin-7-yl)amino)methyl)-6-cyclopropylimidazo[1,2-a]pyridin-8-yl)-3-methylimidazolidine-2,4-dione

Ti(OiPr)₄ (0.079 mL, 0.27 mmol) was added to a mixture was 7-amino-2-((1S,2S)-2-(3-chlorophenyl)cyclopropyl)quinazolin-4 (3H)-one (84 mg, 0.27 mmol) and 6-cyclopropyl-8-(3-methyl-2,4-dioxoimidazolidin-1-yl)imidazo[1,2-a]pyridine-2-carbaldehyde (80 mg, 0.27 mmol) in DCM (0.5 mL) and MeOH (0.5 mL) and heated to 50° C. for 18 h. The reaction was cooled to room temperature and NaCNBH₃ (34 mg, 0.27 mmol) was added. The reaction stirred for 2 h at room temperature, then diluted with water, filtered through Celite® and concentrated in vacuo. The residue was dissolved into DCM, washed with water then brine, passed through a hydrophobic cartridge then concentrated in vacuo. The residue was purified by silica gel column chromatography, eluting with a gradient of 0-10% 7 N NH₃ in MeOH in DCM followed by purification by preparative HPLC to give the title compound (8 mg, 5%).

ESI-MS (M+H)+: 594.4, ¹H NMR (400 MHz, DMSO) δ 11.85 (s, 1H), 8.25 (d, J=1.1 Hz, 1H), 7.74-7.71 (m, 2H), 7.36-7.26 (m, 4H), 7.18-7.11 (m, 2H), 6.79 (dd, J=2.3, 8.8 Hz, 1H), 6.51 (d, J=2.1 Hz, 1H), 4.92 (s, 2H), 4.46 (d, J=5.5 Hz, 2H), 2.97 (s, 3H), 2.56-2.53 (m, 1H), 2.21-2.15 (m, 1H), 1.97-1.90 (m, 1H), 1.77-1.72 (m, 1H), 1.53 (dd, J=4.3, 14.7 Hz, 1H), 0.96-0.91 (m, 2H), 0.66-0.62 (m, 2H). The compound in Table 2 was synthesised using a similar procedure to that described above using rac-(1S*,2S)-2-(4-methylpyrimidin-2-yl)cyclopropane-1-carboxylic acid and methyl 4-amino-6-chloronicotinate.

TABLE 2

| Ex | Compound | Coupling partner | Analytical data |
|---|---|---|---|
| 7 | 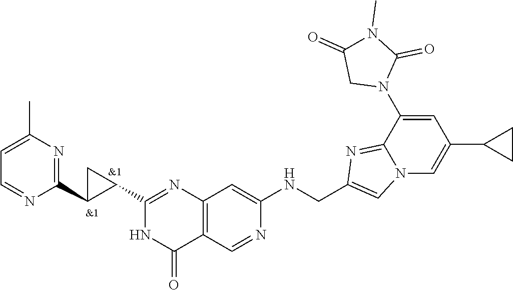 [SMILES: Cc1ccnc([C@H]2C[C@@H]2c2nc3cc(NCc4cn5cc(C6CC6)cc(N6CC(=O)N(C)C6=O)c5n4)ncc3c(=O)[nH]2)n1] | rac-7-amino-2- ((1S*,2S*)-2-(4- methylpyrimidin- 2-yl)cyclopropyl) pyrido[4,3- d]pyrimidin- 4(3H)-one | ESI-MS (M + H):+ 577.4, ¹H NMR (400 MHz, DMSO) δ 8.75 (s, 1H), 8.55 (d, J = 5.1 Hz, 1H), 8.26 (d, J = 1.1 Hz, 1H), 7.73 (d, J = 6.4 Hz, 2H), 7.36 (d, J = 1.5 Hz, 1H), 7.24 (d, J = 5.0 Hz, 1H), 6.69-6.69 (m, 1H), 4.93 (s, 2H), 4.62 (d, J = 4.6 Hz, 2H), 2.99 (s, 3H), 2.82-2.76 (m, 1H), 2.43 (s, 3H), 2.38-2.34 (m, 1H), 1.99- 1.91 (m, 1H), 1.80-1.68 (m, 2H), 0.98-0.92 (m, 2H), 0.68- 0.63 (m, 2H), 1H hidden under DMSO. |
| | I-7 | | |
| | rac-1-(6-cyclopropyl-2-(((2- | | |
| | ((1S*,2S*)-2-(4- | | |
| | methylpyrimidin-2- | | |
| | yl)cyclopropyl)-4-oxo-3,4- | | |
| | dihydropyrido[4,3-d]pyrimidin- | | |
| | 7-yl)amino)methyl)imidazo[1,2- | | |
| | a]pyridin-8-yl)-3-methyl | | |
| | imidazolidine-2,4-dione (I-7) | | |

The compound in Table 3 was synthesised using a similar procedure to that described above using rac-(1S*,2S*)-2-(4-methylpyrimidin-2-yl)cyclopropane-1-carboxylic acid and methyl 2-amino-4-bromobenzoate.

TABLE 3

| Ex | Compound | Coupling partner | Analytical data |
|---|---|---|---|
| 8 | 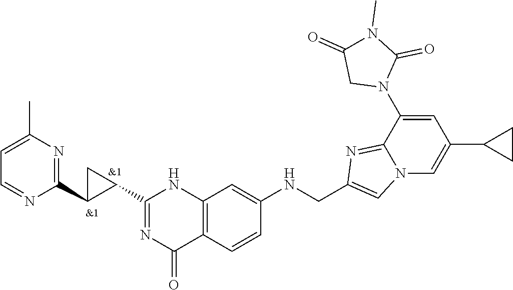 [SMILES: Cc1ccnc([C@H]2C[C@@H]2c2nc(=O)c3ccc(NCc4cn5cc(C6CC6)cc(N6CC(=O)N(C)C6=O)c5n4)cc3[nH]2)n1] | rac-7-amino-2- ((1S*,2S*)-2-(4- methylpyrimidin-2- yl)cyclopropyl) quinazolin- 4(3H)-one | ESI-MS (M + H)+: 576.3, ¹H NMR (400 MHz, DMSO) δ 11.91 (s, 1H), 8.54 (d, J = 5.1 Hz, 1H), 8.28 (d, J = 1.1 Hz, 1H), 7.76-7.73 (m, 2H), 7.37 (d, J = 1.5 Hz, 1H), 7.22 (d, J = 5.4 Hz, 1H), 7.13 (dd, J = 5.8, 5.8 Hz, 1H), 6.81 (dd, J = 2.3, 8.8 Hz, 1H), 6.55 (d, J = 2.3 Hz, 1H), 4.94 (s, 2H), 4.48 (d, J = 5.8 Hz, 2H), 2.99 (s, 3H), 2.78- 2.68 (m, 1H), 2.43 (s, 3H), 1.99-1.91 (m, 1H), 1.76- 1.63 (m, 3H), 0.98-0.92 (m, 2H), 0.69-0.63 (m, 2H). |
| | I-8 | | |
| | rac-1-(6-cyclopropyl-2-(((2- | | |
| | ((1S*,2S*)-2-(4- | | |
| | methylpyrimidin-2- | | |
| | yl)cyclopropyl)-4-oxo-1,4- | | |
| | dihydroquinazolin-7- | | |
| | yl)amino)methyl)imidazo[1,2- | | |
| | a]pyridin-8-yl)-3-methyl | | |
| | imidazolidine-2,4-dione (I-8) | | |

### Example 9

### Synthesis of 1-(6-cyclopropyl-2-(((2-(2-(4-methylpyrimidin-2-yl)cyclopropyl)-4-oxo-1,4-dihydropyrido[3,2-d]pyrimidin-7-yl)amino)methyl)imidazo[1,2-a]pyridin-8-yl)-3-methylimidazolidine-2,4-dione (I-9)

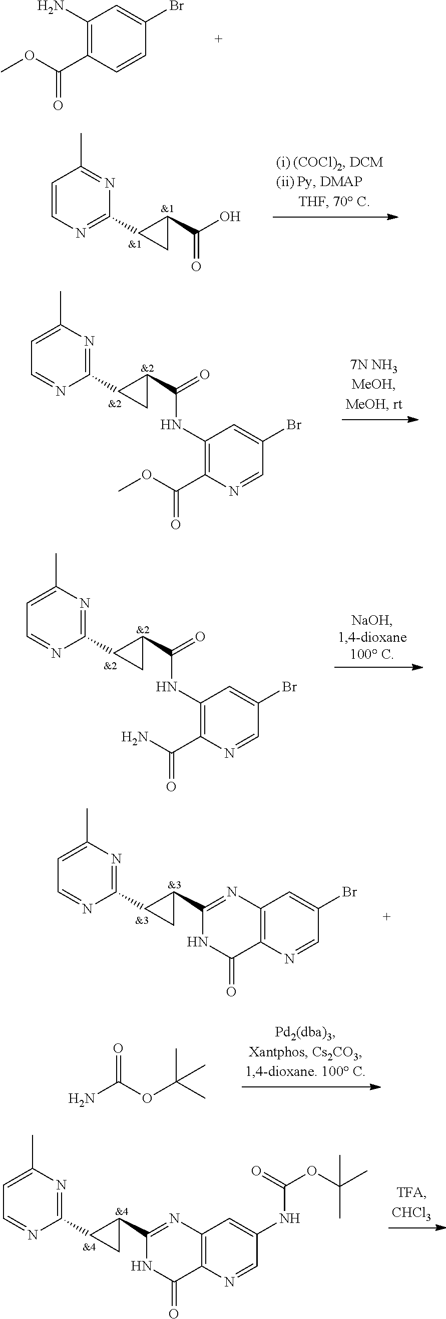

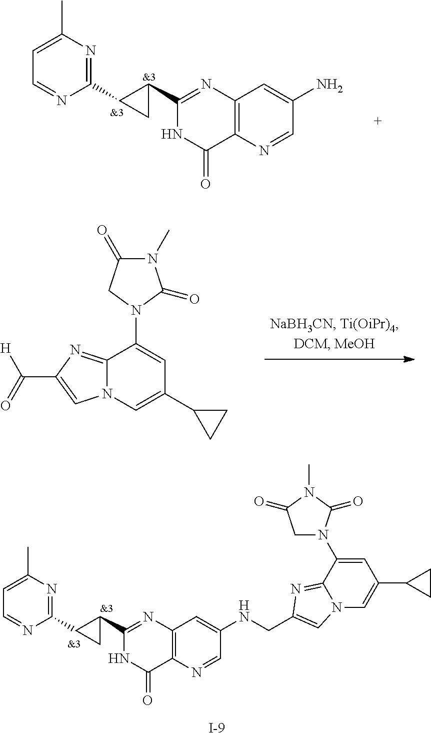

### Synthesis of rac-methyl 5-bromo-3-((1S*,2S*)-2-(4-methylpyrimidin-2-yl)cyclopropane-1-carboxamido)picolinate

Oxalyl chloride (0.78 mL, 8.90 mmol) was added dropwise to a stirred solution of rac-(1S*,2S*)-2-(4-methylpyrimidin-2-yl)cyclopropanecarboxylic acid (793 mg, 4.45 mmol) and DMF (0.050 mL) in THF (10 mL) at 0° C. and stirred at 0° C. for 30 min. The reaction was then concentrated in vacuo. The residue was suspended in THF (20 mL) followed by the addition of methyl 3-amino-5-bromo-pyridine-2-carboxylate (1079 mg, 4.67 mmol), DMAP (109 mg, 0.890 mmol) and pyridine (3.6 mL, 44.5 mmol). The mixture was heated to 70° C. and stirred for 16 h. then cooled to room temperature and diluted with EtOAc and water. The organics were washed with NaHCO₃ (sat. aq.) and brine, dried over MgSO₄ and concentrated in vacuo to give the title compound (1.26 g, 72%) as a black solid.

ESI-MS (M+H)+: 391.2, ¹H NMR (400 MHz, CDCl₃) δ 11.24 (s, 1H), 9.41 (d, J=2.0 Hz, 1H), 8.46-8.43 (m, 2H), 6.99 (d, J=5.1 Hz, 1H), 4.02 (s, 3H), 2.92-2.86 (m, 1H), 2.49 (s, 3H), 2.39-2.34 (m, 1H), 1.80-1.71 (m, 2H).

### Synthesis of rac-5-bromo-3-((1S*,2S*)-2-(4-methylpyrimidin-2-yl)cyclopropane-1-carboxamido)picolinamide

rac-methyl 5-bromo-3-((1S*,2S*)-2-(4-methylpyrimidin-2-yl)cyclopropane-1-carboxamido)picolinate (1.26 g, 3.22 mmol) was suspended in 7 N NH₃ in MeOH (26 mL) and stirred at 65° C. for 18 h. The reaction was cooled to room temperature and concentrated in vacuo to give the title compound (1.2 g, 67%).

ESI-MS (M+H)+: 376.1, ¹H NMR (400 MHz, CDCl₃) δ 12.18 (s, 1H), 9.39 (d, J=2.0 Hz, 1H), 8.43 (d, J=5.1 Hz, 1H), 8.26 (d, J=2.0 Hz, 1H), 8.00-8.00 (m, 1H), 6.97 (d, J=5.4 Hz, 1H), 5.56 (s, 1H), 2.88-2.83 (m, 1H), 2.47 (s, 3H), 2.39-2.34 (m, 1H), 1.78-1.69 (m, 2H).

### Synthesis of rac-7-bromo-2-((1S*,2S*)-2-(4-methylpyrimidin-2-yl)cyclopropyl)pyrido[3,2-d]pyrimidin-4 (3H)-one

NaOH (383 mg, 9.57 mmol) (azetroped 3× with toluene) was added to a stirred mixture of rac-5-bromo-3-((1S*,2S*)-2-(4-methylpyrimidin-2-yl)cyclopropane-1-carboxamido)picolinamide (1200 mg, 3.19 mmol) in 1,4-dioxane (10 mL), the reaction was then stirred at 100° C. for 18 h. The reaction was cooled to room temperature and partitioned between water and EtOAc. The aqueous layer was extracted with EtOAc (3×). The combined organics were dried over MgSO₄ and concentrated in vacuo to give the title compound (0.9 g, 79%) as an off-white solid.

ESI-MS (M+H)+: 358.1 ¹H NMR (400 MHz, DMSO) δ 12.87 (s, 1H), 8.79 (d, J=2.1 Hz, 1H), 8.57 (d, J=5.1 Hz, 1H), 8.30 (d, J=2.1 Hz, 1H), 7.25 (d, J=5.0 Hz, 1H), 2.89-2.84 (m, 1H), 2.69-2.63 (m, 1H), 2.45 (s, 3H), 1.85-1.74 (m, 2H).

### Synthesis of rac-tert-butyl (2-((1S*,2S*)-2-(4-methylpyrimidin-2-yl)cyclopropyl)-4-oxo-3,4-dihydropyrido[3,2-d]pyrimidin-7-yl)carbamate

rac-7-Bromo-2-((1S*,2S*)-2-(4-methylpyrimidin-2-yl)cyclopropyl)pyrido[3,2-d]pyrimidin-4 (3H)-one (400 mg, 1.12 mmol), tert-butyl carbamate (196 mg, 1.68 mmol), Pd(OAc)₂ (13 mg, 0.056 mmol), Xantphos (65 mg, 0.11 mmol) and Cs₂CO₃ (1092 mg, 3.35 mmol) were suspended in 1,4-dioxane (5 mL) and degassed with N₂ for 5 min. The reaction was then heated to 100° C. and stirred for 16 h. Further tert-butyl carbamate (196 mg, 1.68 mmol), Pd(OAc)₂ (13 mg, 0.056 mmol) and Xantphos (65 mg, 0.11 mmol) were added and the mixture was stirred at 100° C. for a further 24 h. The reaction was cooled to room temperature and filtered through Celite®. The residue was purified by column chromatography on silica gel, eluting with 0-10% MeOH in DCM to give the title compound (220 mg, 60%) as a yellow solid. ESI-MS (M+H)+: 395.3

### Synthesis of rac-7-amino-2-((1S*2S*)-2-(4-methylpyrimidin-2-yl)cyclopropyl)pyrido[3,2-d]pyrimidin-4 (3H)-one

TFA (0.43 mL, 5.58 mmol) was added to a stirred solution of rac-tert-butyl (2-((1S*,2S*)-2-(4-methylpyrimidin-2-yl)cyclopropyl)-4-oxo-3,4-dihydropyrido[3,2-d]pyrimidin-7-yl)carbamate (220 mg, 0.56 mmol) in chloroform (6 mL) and stirred at room temperature for 16 h. The reaction was concentrated in vacuo and the residue was loaded onto an SCX cartridge, which was washed with 3:1 DCM:MeOH. The compound was released using 7N NH₃ in MeOH in DCM to give the title compound (150 mg) which was carried forward to the next step without further purification.

ESI-MS (M+H)+: 295.2, ¹H NMR (400 MHz, DMSO) δ 8.58-8.55 (m, 1H), 8.09 (d, J=2.5 Hz, 1H), 7.24 (dd, J=4.8, 4.8 Hz, 2H), 6.78 (d, J=2.5 Hz, 1H), 6.28 (s, 2H), 3.18 (d, J=5.3 Hz, 1H), 2.81-2.75 (m, 1H), 2.58-2.54 (m, 1H), 2.44 (d, J=2.3 Hz, 3H), 1.83-1.67 (m, 2H).

### Synthesis of rac-1-(6-cyclopropyl-2-(((2-((1S*,2S*)-2-(4-methylpyrimidin-2-yl)cyclopropyl)-4-oxo-3,4-dihydropyrido[3,2-d]pyrimidin-7-yl)amino)methyl)imidazo[1,2-a]pyridin-8-yl)-3-methylimidazolidine-2,4-dione (I-9)

Using a similar method to that used for 1-(2-(((2-((1S,2S)-2-(3-chlorophenyl)cyclopropyl)-4-oxo-3,4-dihydroquinazolin-7-yl)amino)methyl)-6-cyclopropylimidazo[1,2-a]pyridin-8-yl)-3-methylimidazolidine-2,4-dione, the title compound (17.9 mg, 6%) was prepared from rac-7-amino-2-((1S*,2S*)-2-(4-methylpyrimidin-2-yl)cyclopropyl)pyrido[3,2-d]pyrimidin-4 (3H)-one and 6-cyclopropyl-8-(3-methyl-2,4-dioxoimidazolidin-1-yl)imidazo[1,2-a]pyridine-2-carbaldehyde.

ESI-MS (M+H):+577.4, ¹H NMR (400 MHz, DMSO) δ 12.19 (s, 1H), 8.55 (d, J=5.1 Hz, 1H), 8.28 (d, J=1.1 Hz, 1H), 8.24 (d, J=2.6 Hz, 1H), 7.81 (s, 1H), 7.44 (dd, J=6.0, 6.0 Hz, 1H), 7.37 (d, J=1.5 Hz, 1H), 7.23 (d, J=5.1 Hz, 1H), 6.81 (d, J=2.5 Hz, 1H), 4.93 (s, 2H), 4.51 (d, J=5.9 Hz, 2H), 2.99 (s, 3H), 2.80-2.74 (m, 1H), 2.56 (dd, J=4.5, 4.5 Hz, 1H), 2.43 (s, 3H), 1.99-1.92 (m, 1H), 1.77-1.65 (m, 2H), 0.98-0.93 (m, 2H), 0.69-0.64 (m, 2H).

### Example 10

### Synthesis of rac-1-(6-cyclopropyl-2-(((2-((1S*,2S*)-2-(4-methylpyrimidin-2-yl)cyclopropyl)-4-oxo-1,4-dihydro-1,6-naphthyridin-7-yl)amino)methyl)imidazo[1,2-a]pyridin-8-yl)-3-methylimidazolidine-2,4-dione (I-10)

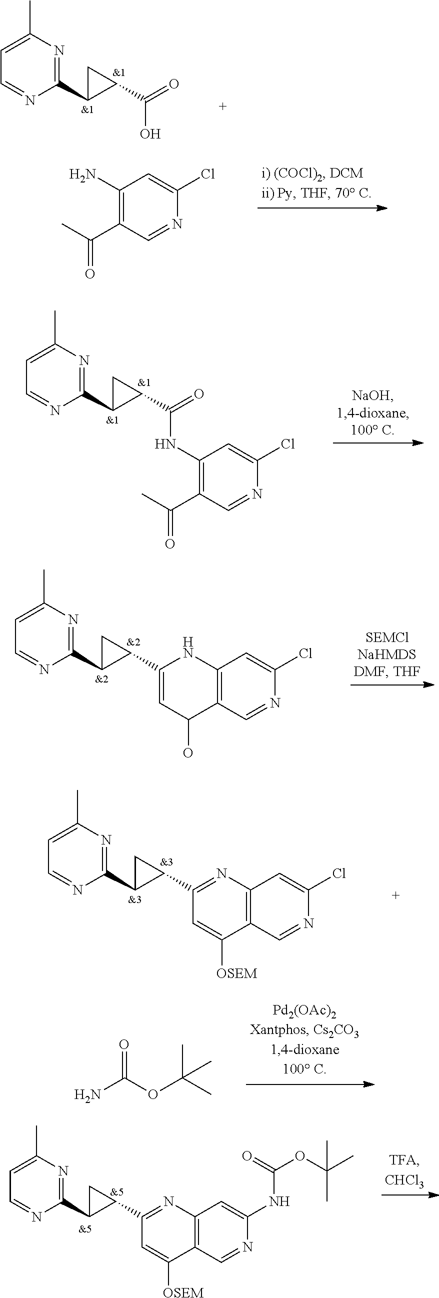

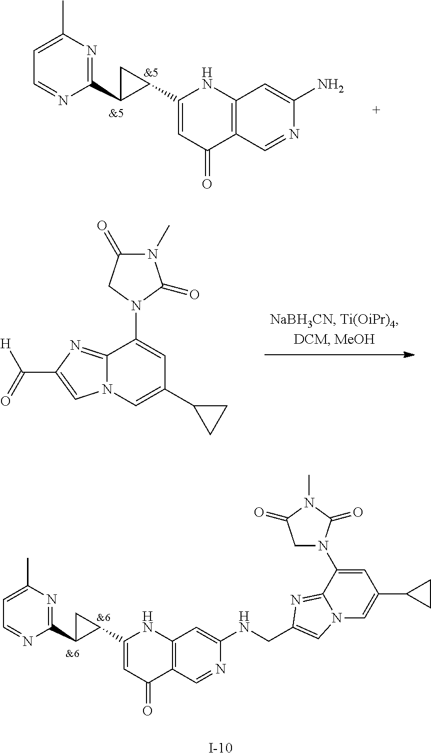

### Synthesis of rac-(1S*,2S*)—N-(5-acetyl-2-chloropyridin-4-yl)-2-(4-methylpyrimidin-2-yl)cyclopropane-1-carboxamide

Oxalyl chloride (0.98 mL, 11.2 mmol) was added dropwise to a stirred solution of 1-(4-amino-6-chloro-3-pyridyl)ethanone (1.00 g, 5.89 mmol) and DMF (0.050 mL) in THF (10 mL) at 0° C. and stirred at 0° C. for 30 min. The reaction was then concentrated to dryness then suspended in THF (10 mL) followed by the addition of rac-(1S*,2S*)-2-(4-methylpyrimidin-2-yl)cyclopropane-1-carboxylic acid (1.00 g, 5.61 mmol), and pyridine (4.5 mL, 56.1 mmol). The reaction was then heated to 70° C. and stirred for 16 h. The reaction was cooled to room temperature and diluted with EtOAc. The reaction was washed with NaHCO₃ (sat. aq.) and brine. The organics were dried over MgSO₄ and concentrated in vacuo. The residue was purified by column chromatography on silica gel, eluting with 0-100% EtOAc in cyclohexane to give the title compound (900 mg, 95%) as a pale blue solid.

ESI-MS (M+H)+: 331.1, ¹H NMR (400 MHz, CDCl₃) δ 12.10-12.05 (m, 1H), 8.85 (s, 1H), 8.76 (s, 1H), 8.44 (d, J=5.1 Hz, 1H), 6.98 (d, J=4.8 Hz, 1H), 2.91-2.84 (m, 1H), 2.68 (s, 3H), 2.48 (s, 3H), 2.39-2.33 (m, 1H), 1.81-1.72 (m, 2H).

### Synthesis of rac-7-chloro-2-((1S*,2S*)-2-(4-methylpyrimidin-2-yl)cyclopropyl)-1,6-naphthyridin-4 (1H)-one

NaOH (435 mg, 10.9 mmol) (which had been azeotroped with toluene×3) was added to a stirred suspension of rac-(1S*,2S*)—N-(5-acetyl-2-chloropyridin-4-yl)-2-(4-methylpyrimidin-2-yl)cyclopropane-1-carboxamide (900 mg, 2.72 mmol) in 1,4-dioxane (20 mL). The reaction was then heated to 100° C. for 1 h. The mixture was diluted with water and extracted with EtOAc. The organics were washed with NaHCO₃ (sat. aq.) then brine, dried over MgSO₄ then concentrated in vacuo to give the title compound (400 mg, 87%) as a pale yellow solid.

ESI-MS (M+H)+: 313.1, ¹H NMR (400 MHz, DMSO) δ 11.92 (s, 1H), 8.95 (s, 1H), 8.58 (d, J=5.1 Hz, 1H), 7.44 (s, 1H), 7.25 (d, J=5.4 Hz, 1H), 6.08 (s, 1H), 2.73-2.67 (m, 1H), 2.45 (s, 3H), 1.86-1.72 (m, 2H), 1H obscured by DMSO signal.

### Synthesis of rac-7-chloro-2-((1S*,2S*)-2-(4-methylpyrimidin-2-yl)cyclopropyl)-4-((2-(trimethylsilyl)ethoxy)methoxy)-1,6-naphthyridine

NaHMDS (1 M in THF, 1.2 mL, 1.25 mmol) was added dropwise to a stirred suspension of rac-7-chloro-2-((1S*,2S*)-2-(4-methylpyrimidin-2-yl)cyclopropyl)-1,6-naphthyridin-4(H)-one (390 mg, 1.25 mmol) in DMF (3.00 mL) at room temperature and stirred for 10 min. SEM-Cl (0.22 mL, 1.25 mmol) was then added and the reaction was stirred at room temperature for 18 h. The reaction was quenched with water then extracted with EtOAc. The combined organics were washed with NaHCO₃ (sat. aq.) and brine, dried over MgSO₄ and concentrated in vacuo. The residue was purified by column chromatography on silica gel, eluting with 0-100% EtOAc in cyclohexane to give the title compound (300 mg, 54%) as an orange oil.

ESI-MS (M+H)+: 443.3, ¹H NMR (400 MHz, CDCl₃) δ 9.26 (s, 1H), 8.46 (d, J=5.1 Hz, 1H), 7.75 (s, 1H), 7.03 (s, 1H), 6.97 (d, J=5.1 Hz, 1H), 5.48 (d, J=1.8 Hz, 2H), 3.85-3.78 (m, 2H), 3.05-2.98 (m, 1H), 2.88-2.78 (m, 1H), 2.49 (s, 3H), 2.06-1.90 (m, 2H), 1.01-0.95 (m, 2H), 0.00 (s, 9H).

### Synthesis of rac-tert-butyl (2-((1S*,2S*)-2-(4-methylpyrimidin-2-yl)cyclopropyl)-4-((2-(trimethylsilyl)ethoxy)methoxy)-1,6-naphthyridin-7-yl)carbamate

rac-7-chloro-2-((1S*,2S*)-2-(4-methylpyrimidin-2-yl)cyclopropyl)-4-((2-(trimethylsilyl)ethoxy)methoxy)-1,6-naphthyridine (300 mg, 0.68 mmol), tert-butyl carbamate (119 mg, 1.02 mmol), Pd(OAc)₂ (7.6 mg, 0.034 mmol), Xantphos (39 mg, 0.068 mmol) and Cs₂CO₃ (662 mg, 2.03 mmol) were suspended in 1,4-dioxane (5.0 mL) and purged with N₂ for 5 min. The reaction was then heated to 100° C. and stirred for 1 h. The mixture was filtered through Celite® then concentrated in vacuo. The residue was purified by column chromatography on silica gel, eluting with 0-100% EtOAc in cyclohexane to give the title compound (300 mg, 84%) as a white solid.

ESI-MS (M+H)+: 524.4, ¹H NMR (400 MHz, CDCl₃) δ 9.14 (s, 1H), 8.44 (d, J=5.1 Hz, 1H), 8.23 (s, 1H), 7.40 (s, 1H), 6.94 (d, J=5.1 Hz, 1H), 6.86 (s, 1H), 5.44 (d, J=1.9 Hz, 2H), 3.83-3.78 (m, 2H), 2.99-2.94 (m, 1H), 2.78-2.72 (m, 1H), 2.47 (s, 3H), 2.08-2.03 (m, 1H), 1.90-1.84 (m, 1H), 1.46 (s, 9H), 1.43-1.40 (m, 1H), 1.30-1.21 (m, 1H), 1.00-0.95 (m, 2H), −0.01 (s, 9H).

### Synthesis of rac-7-amino-2-((1S*,2S*)-2-(4-methylpyrimidin-2-yl)cyclopropyl)-1,6-naphthyridin-4 (1H)-one

TFA (0.44 mL, 5.73 mmol) was added to a solution rac-tert-butyl (2-((1S*,2S*)-2-(4-methylpyrimidin-2-yl)cyclopropyl)-4-((2-(trimethylsilyl)ethoxy)methoxy)-1,6-naphthyridin-7-yl)carbamate (300 mg, 0.573 mmol) in CHCl₃ (4.00 mL), the resulting solution was stirred at room temperature for 4 h. The reaction was concentrated in vacuo. The residue was loaded onto a SCX cartridge which was washed with DCM in MeOH and eluted with 7 N NH₃ in MeOH in DCM to give the title compound (100 mg, 60%) of product as a pale orange solid.

ESI-MS (M+H)+: 294.2, ¹H NMR (400 MHz, DMSO) δ 11.14 (s, 1H), 8.63 (s, 1H), 8.57 (d, J=5.1 Hz, 1H), 7.24 (d, J=5.0 Hz, 1H), 6.39 (s, 2H), 6.22 (s, 1H), 5.64 (d, J=1.8 Hz, 1H), 2.62-2.57 (m, 1H), 2.45 (s, 4H), 1.75-1.66 (m, 2H).

### Synthesis of rac-1-(6-cyclopropyl-2-(((2-((1S*,2S*)-2-(4-methylpyrimidin-2-yl)cyclopropyl)-4-oxo-1,4-dihydro-1,6-naphthyridin-7-yl)amino)methyl)imidazo[1,2-a]pyridin-8-yl)-3-methylimidazolidine-2,4-dione

Ti(OiPr)₄ (0.20 mL, 0.682 mmol) was added to a stirred solution of rac-7-amino-2-((1S*,2S*)-2-(4-methylpyrimidin-2-yl)cyclopropyl)-1,6-naphthyridin-4(H)-one (40 mg, 0.136 mmol) and 6-cyclopropyl-8-(3-methyl-2,4-dioxo-imidazolidin-1-yl)imidazo[1,2-a]pyridine-2-carbaldehyde (41 mg, 0.136 mmol) in a 1:1 mixture of MeOH (2.0 mL) and DCM (2.0 mL). The mixture was then heated at 50° C. in a sealed tube for 12 h. The reaction was cooled to room temperature. NaCNBH₃ (30 mg, 0.477 mmol) was added then the mixture was stirred at room temperature for 18 h. The reaction was diluted with water (1.0 mL), filtered through Celite® and concentrated in vacuo. The residue was purified by column chromatography on silica gel, eluting with 0-10% (7N NH₃ in MeOH) in DCM followed by preparative HPLC to give the title compound (10.6 mg, 12%).

ESI-MS (M+H):+576.4, ¹H NMR (400 MHz, DMSO) δ 11.15 (s, 1H), 8.70 (s, 1H), 8.56 (d, J=5.1 Hz, 1H), 8.24 (d, J=1.1 Hz, 1H), 7.69 (s, 1H), 7.48 (dd, J=6.0, 6.0 Hz, 1H), 7.34 (d, J=1.5 Hz, 1H), 7.23 (d, J=5.1 Hz, 1H), 6.28 (s, 1H), 5.67 (s, 1H), 4.91 (s, 2H), 4.57 (d, J=5.9 Hz, 2H), 2.99 (s, 3H), 2.61-2.56 (m, 1H), 2.46-2.41 (m, 4H), 1.98-1.90 (m, 1H), 1.75-1.64 (m, 2H), 0.97-0.92 (m, 2H), 0.67-0.62 (m, 2H).

### Examples 11-13

### Synthesis of rac-1-(6-cyclopropyl-2-(((2-((1S*,2S*)-2-(4-methylpyrimidin-2-yl)cyclopropyl)-4-oxo-1,4-dihydroquinolin-7-yl)amino)methyl)imidazo[1,2-a]pyridin-8-yl)-3-methylimidazolidine-2,4-dione (I-11)

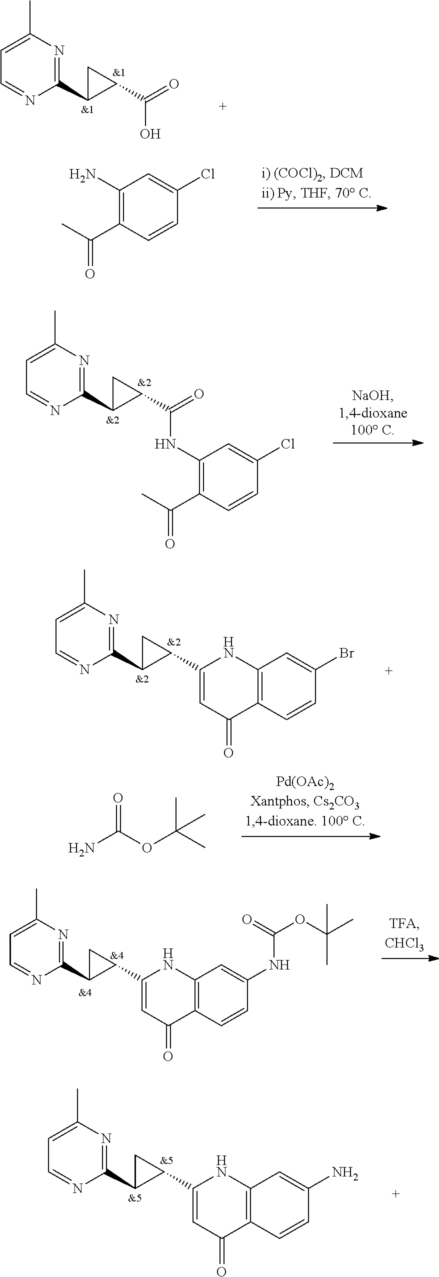

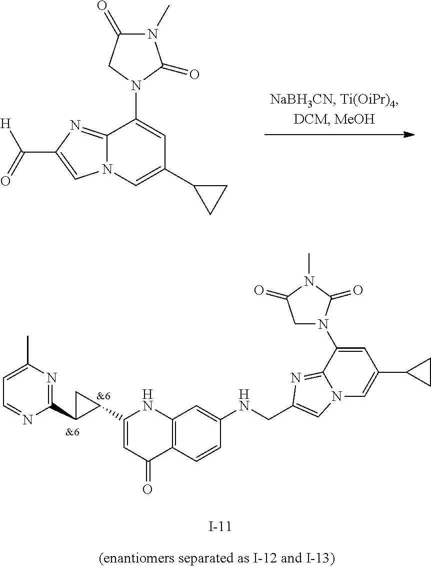

### Synthesis of rac-(1S*,2S*)—N-(2-acetyl-5-chlorophenyl)-2-(4-methylpyrimidin-2-yl)cyclopropane-1-carboxamide

Oxalyl chloride (0.71 mL, 5.61 mmol) was added dropwise to a stirred solution of 1-(2-amino-4-bromophenyl)ethan-1-one (631 mg, 2.95 mmol) and DMF (0.050 mL) in THF (20 mL) at 0° C. and stirred at 0° C. for 30 min. The reaction was then concentrated to dryness. The residue was suspended in THF (20 mL) followed by the addition of rac-(1S*,2S*)-2-(4-methylpyrimidin-2-yl)cyclopropane-1-carboxylic acid (500 mg, 2.81 mmol), and pyridine (2.3 mL, 28.1 mmol). The reaction was then heated to 70° C. and stirred for 16 h. The reaction was cooled to room temperature and diluted with EtOAc and then washed with NaHCO₃ (sat. aq.) and brine. The organics were dried over MgSO₄ and concentrated in vacuo. The residue was purified by column chromatography on silica gel, eluting with 0-100% EtOAc in cyclohexane to give the title compound (890 mg, 80%).

ESI-MS (M+H)+: 376.1, ¹H NMR (400 MHz, DMSO) δ 11.52-11.50 (m, 1H), 8.57-8.55 (m, 1H), 8.50-8.48 (m, 1H), 7.93-7.90 (m, 1H), 7.46-7.43 (m, 1H), 7.26-7.23 (m, 1H), 2.60-2.59 (m, 4H), 2.45-2.44 (m, 3H), 2.37-2.31 (m, 1H), 1.61-1.56 (m, 2H). From ED01395-188

### Synthesis of rac-7-bromo-2-((1S*,2S*)-2-(4-methylpyrimidin-2-yl)cyclopropyl)quinolin-4 (1H)-one

NaOH (435 mg, 10.9 mmol) (which had been azeotroped with toluene×3) was added to a stirred suspension of rac-(1S*,2S*)—N-(2-acetyl-5-bromophenyl)-2-(4-methylpyrimidin-2-yl)cyclopropane-1-carboxamide (890 mg, 2.38 mmol) in 1,4-dioxane (15 mL). The reaction was then heated to 100° C. and stirred for 1 h. then cooled to room temperature and quenched with NH₄Cl (sat. aq.). The precipitate that formed was filtered, washed with water and dried under vacuum to give the title compound (680 mg, 87%) as a brown solid.

ESI-MS (M+H)+: 358.1, ¹H NMR (400 MHz, DMSO) δ 8.59-8.57 (m, 1H), 7.97-7.94 (m, 1H), 7.75-7.73 (m, 1H), 7.46-7.42 (m, 1H), 7.26-7.24 (m, 1H), 5.96 (s, 1H), 2.71-2.66 (m, 1H), 2.46 (s, 3H), 1.83-1.71 (m, 2H). 1H missing (assumed under DMSO peak), 1H exchangeable not observed.

### Synthesis of rac-tert-butyl (2-((1S*,2S*)-2-(4-methylpyrimidin-2-yl)cyclopropyl)-4-oxo-1,4-dihydroquinolin-7-yl)carbamate

rac-7-bromo-2-((1S*,2S*)-2-(4-methylpyrimidin-2-yl)cyclopropyl)quinolin-4 (1H)-one (340 mg, 0.954 mmol), tert-butyl carbamate (168 mg, 1.43 mmol), Pd(OAc)₂ (21 mg, 0.095 mmol), Xantphos (110 mg, 0.191 mmol) and Cs₂CO₃ (933 mg, 2.86 mmol) were suspended in 1,4-dioxane (10 mL) and purged with N₂ for 5 min. The reaction was then heated to 100° C. and stirred for 1 h. The mixture was filtered through Celite®, concentrated in vacuo and the residue was purified by column chromatography on silica gel, eluting with 0-10% DCM in MeOH to give the title compound (150 mg, ˜60% pure). This was carried forward to the next step without further purification.

ESI-MS (M+H)+: 393.3

### Synthesis of rac-7-amino-2-((1S*,2S*)-2-(4-methylpyrimidin-2-yl)cyclopropyl)quinolin-4 (1H)-one

TFA (0.35 mL, 4.59 mmol) was added to a stirred solution of rac-tert-butyl (2-((1S*,2S*)-2-(4-methylpyrimidin-2-yl)cyclopropyl)-4-oxo-1,4-dihydroquinolin-7-yl)carbamate (60%, 150 mg, 0.229 mmol) in chloroform (4.00 mL) and stirred at room temperature for 30 min. The reaction was concentrated in vacuo. The residue was loaded onto a SCX cartridge which was washed with 3:1 DCM:MeOH and eluted with 7 N NH₃ in MeOH in DCM to give 100 mg of desired product at ˜60% purity. The material was purified by silica gel column chromatography eluting with a gradient of 0-100% EtOAc in cyclohexane to the title compound (50 mg, 43%) as an orange solid.

ESI-MS (M+H)+: 293.3, ¹H NMR (400 MHz, DMSO) δ 11.03 (s, 1H), 8.56 (d, J=5.1 Hz, 1H), 7.68 (d, J=8.7 Hz, 1H), 7.23 (d, J=5.0 Hz, 1H), 6.53 (dd, J=2.1, 8.7 Hz, 1H), 6.46 (d, J=2.0 Hz, 1H), 5.82 (s, 2H), 5.58 (d, J=1.8 Hz, 1H), 2.58-2.54 (m, 1H), 2.45 (s, 3H), 1.71-1.66 (m, 2H). 1H obscured by DMSO signal.

### Synthesis of rac-1-(6-cyclopropyl-2-(((2-((1S*,2S*)-2-(4-methylpyrimidin-2-yl)cyclopropyl)-4-oxo-1,4-dihydroquinolin-7-yl)amino)methyl)imidazo[1,2-a]pyridin-8-yl)-3-methylimidazolidine-2,4-dione

Ti(OiPr)₄ (0.25 mL, 0.855 mmol) was added to a stirred solution of rac-7-amino-2-((1S*,2S*)-2-(4-methylpyrimidin-2-yl)cyclopropyl)quinolin-4 (1H)-one (50 mg, 0.171 mmol, 1.00 eq) and 6-cyclopropyl-8-(3-methyl-2,4-dioxoimidazolidin-1-yl)imidazo[1,2-a]pyridine-2-carbaldehyde (51 mg, 0.171 mmol, 1.00 eq) in a 1:1 mixture of MeOH (2.0 mL) and DCM (2.0 mL). The reaction was then heated at 50° C. in a sealed tube for 5 h. LCMS showed good conversion to imine. The reaction was cooled to room temperature. NaCNBH₃ (38 mg, 0.599 mmol) was added then the reaction was stirred at room temperature for 1 h. Quenched with water (1.0 mL) and filtered through Celite®. Washed through with MeOH (3×5 mL), concentrated to give crude residue. The residue was purified by column chromatography on silica gel, eluting with 0-20% (7N NH₃ in MeOH) in DCM followed by preparative HPLC to give the title compound (28 mg, 28%) as a mixture of enantiomers.

ESI-MS (M+H)+: 575.4, ¹H NMR (400 MHz, DMSO) δ 11.03 (s, 1H), 8.55 (d, J=5.0 Hz, 1H), 8.26-8.25 (m, 1H), 7.74-7.70 (m, 2H), 7.34 (d, J=1.6 Hz, 1H), 7.22 (d, J=5.1 Hz, 1H), 6.99-6.95 (m, 1H), 6.70 (dd, J=2.1, 8.9 Hz, 1H), 6.45 (d, J=2.1 Hz, 1H), 5.61 (d, J=1.6 Hz, 1H), 4.92 (s, 2H), 4.45-4.41 (m, 2H), 2.99 (s, 3H), 2.44-2.43 (m, 4H), 1.98-1.90 (m, 1H), 1.71-1.62 (m, 2H), 0.97-0.91 (m, 2H), 0.67-0.62 (m, 2H). 1H missing, assumed under DMSO.

SFC separation (LUX Cellulose-4 21.2×250 mm, 5 um 45/55 EtOH (0.1% DEA)/CO₂, 100 mL/min, 120 bar, 40° C.) of the enantiomers of 1-(6-cyclopropyl-2-(((2-(2-(4-methylpyrimidin-2-yl)cyclopropyl)-4-oxo-1,4-dihydroquinolin-7-yl)amino)methyl)imidazo[1,2-a]pyridin-8-yl)-3-methylimidazolidine-2,4-dione (i.e., 1-(6-cyclopropyl-2-(((2-((1S,2S)-2-(4-methylpyrimidin-2-yl)cyclopropyl)-4-oxo-1,4-dihydroquinolin-7-yl)amino)methyl)imidazo[1,2-a]pyridin-8-yl)-3-methylimidazolidine-2,4-dione and 1-(6-cyclopropyl-2-(((2-((1R,2R)-2-(4-methylpyrimidin-2-yl)cyclopropyl)-4-oxo-1,4-dihydroquinolin-7-yl)amino)methyl)imidazo[1,2-a]pyridin-8-yl)-3-methylimidazolidine-2,4-dione) gave:

1st eluting isomer: (4.4 mg) (Example 12, I-12)

ESI-MS (M+H)+: 575.4, ¹H NMR (400 MHz, DMSO) δ 11.03 (s, 1H), 8.55 (d, J=5.1 Hz, 1H), 8.26 (d, J=1.1 Hz, 1H), 7.72 (d, J=8.6 Hz, 1H), 7.70 (s, 1H), 7.34 (d, J=1.5 Hz, 1H), 7.22 (d, J=5.1 Hz, 1H), 6.97 (dd, J=5.8, 5.8 Hz, 1H), 6.70 (dd, J=2.1, 8.9 Hz, 1H), 6.45 (d, J=2.0 Hz, 1H), 5.62 (s, 1H), 4.92 (s, 2H), 4.43 (d, J=5.5 Hz, 2H), 2.99 (s, 3H), 2.56-2.54 (m, 1H), 2.43 (s, 4H), 1.98-1.90 (m, 1H), 1.71-1.62 (m, 2H), 0.97-0.91 (m, 2H), 0.67-0.62 (m, 2H). Enatiomer RT=20.7

2nd eluting isomer: (4.4 mg) (Example 13, I-13)

ESI-MS (M+H)+: 575.4, ¹H NMR (400 MHz, DMSO) δ 11.05 (s, 1H), 8.57-8.53 (m, 1H), 8.26 (d, J=1.4 Hz, 1H), 7.72 (d, J=8.8 Hz, 1H), 7.70 (s, 1H), 7.34 (d, J=1.5 Hz, 1H), 7.21 (d, J=5.0 Hz, 1H), 6.97 (s, 1H), 6.71-6.70 (m, 1H), 6.45 (d, J=1.8 Hz, 1H), 5.61 (s, 1H), 4.92 (s, 2H), 4.42 (d, J=5.6 Hz, 2H), 2.99 (s, 3H), 2.58-2.55 (m, 1H), 2.43 (s, 4H), 1.98-1.90 (m, 1H), 1.70-1.65 (m, 2H), 0.97-0.91 (m, 2H), 0.67-0.62 (m, 2H). Enantiomer RT=27.4

### Example 14

### Synthesis of rac-1-(6-cyclopropyl-2-(((6-((1S*,2S*)-2-(4-methylpyrimidin-2-yl)cyclopropyl)-1,5-naphthyridin-3-yl)amino)methyl)imidazo[1,2-a]pyridin-8-yl)-3-methylimidazolidine-2,4-dione (I-14)

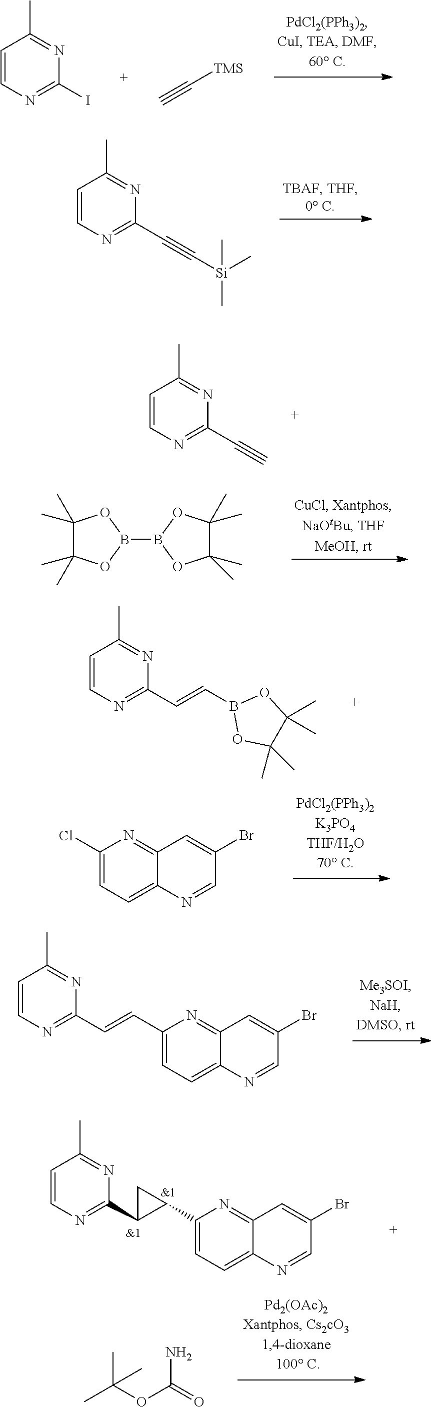

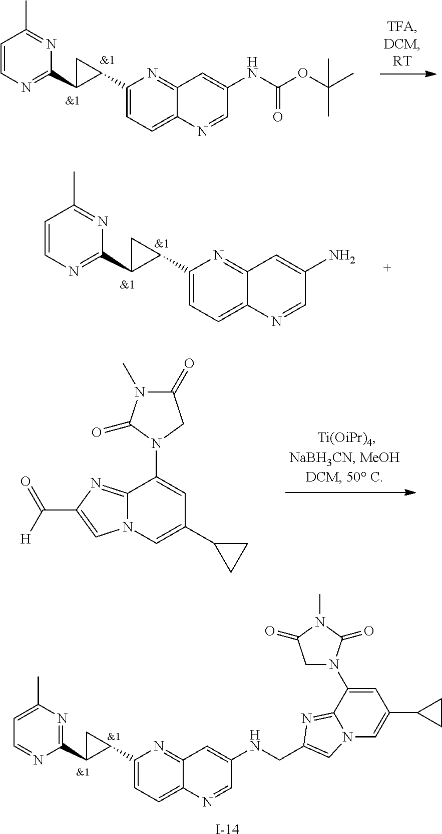

### Synthesis of 4-methyl-2-((trimethylsilyl)ethynyl)pyrimidine

PdCl₂(PPh₃)₂ (3.55 g, 5.06 mmol) was added to a N₂ purged solution of 2-chloro-4-methylpyrimidine (13 g, 101.12), NEt₃ (131.2 mL, 941.4 mmol), TMS-acetylene (16.8 mL, 121.35 mmol) and CuI (1.93 g, 10.11 mmol) in DMF (13 mL) and the mixture was heated to 60° C. for 18 h. The reaction was diluted with EtOAc and filtered through Celite®. The organics were washed with NaHCO₃ (sat. aq., 2×200 mL) then brine (100 mL), dried over MgSO₄ then concentrated in vacuo. The residue was purified by column chromatography on silica gel, eluting with 20-35% EtOAc in cyclohexane to give the title compound (6.98 g, 36%).

ESI-MS (M+H)+: 191.2, ¹H NMR (400 MHz, CDCl₃) δ 8.55 (d, J=5.1 Hz, 1H), 7.10 (d, J=5.1 Hz, 1H), 2.54 (s, 3H), 0.29 (s, 9H).

### Synthesis of 2-ethynyl-4-methylpyrimidine

TBAF (1 M, 44 mL, 44.1 mmol) was added dropwise to a cooled solution of 4-methyl-2-((trimethylsilyl)ethynyl)pyrimidine (7.00 g, 36.8 mmol) in THF (75 mL) at 0° C. and reaction mixture was stirred for 45 min. The reaction mixture was allowed to warm to room temperature, quenched with water (75 mL) and extracted with EtOAc (3×200 mL). The combined organics were dried over MgSO₄, filtered and concentrated in vacuo. The residue was purified by column chromatography on silica gel, eluting with 20-85% EtOAc in cyclohexane to give the title compound (2.88 g, 66.3%) as a pale yellow solid.

ESI-MS (M+H)+: 119.3, ¹H NMR (400 MHz, CDCl₃) δ 8.57 (d, J=5.1 Hz, 1H), 7.15 (d, J=5.1 Hz, 1H), 3.11 (s, 1H), 2.55 (s, 3H).

### Synthesis of (E)-4-methyl-2-(2-(4,4,5,5-tetramethyl-1,3,2-dioxaborolan-2-yl)vinyl)pyrimidine

CuCl (60 mg, 0.609 mmol), Xantphos (353 mg, 0.609 mmol) and NaO^{t}Bu (117 mg, 1.22 mmol) were suspended in THF (8.0 mL) and stirred at room temperature for 30 min. (BPin)₂ (3.25 g, 12.8 mmol) in THF (4.0 mL) was added and the mixture was stirred at room temperature for 10 min. 2-Ethynyl-4-methylpyrimidine (1.44 g, 12.2 mmol) in THF (3.0 mL) and MeOH (0.99 mL, 24.4 mmol) were added and the reaction mixture was stirred at room temperature for 18 h. The reaction mixture was quenched with water (50 mL) and extracted with EtOAc (3×100 mL). The combined organic phases were washed with brine (100 mL), dried over MgSO₄, filtered and concentrated in vacuo. The residue was purified by column chromatography on silica gel, eluting with 0-40% EtOAc in cyclohexane to give the title compound (2.53 g, 76%) as a pale yellow oil, which solidified on standing.

ESI-MS (M+H)+: 247.3, ¹H NMR (400 MHz, CDCl₃) δ 8.57 (d, J=5.1 Hz, 1H), 7.46 (d, J=18.2 Hz, 1H), 7.01 (m, 2H), 2.52 (s, 3H), 1.30 (s, 12H).

### Synthesis of (E)-7-bromo-2-(2-(4-methylpyrimidin-2-yl)vinyl)-1,5-naphthyridine

7-bromo-2-chloro-1,5-naphthyridine (470 mg, 1.93 mmol), (E)-4-methyl-2-(2-(4,4,5,5-tetramethyl-1,3,2-dioxaborolan-2-yl)vinyl)pyrimidine (475 mg, 1.93 mmol), PdCl₂(PPh₃)₂ (68 mg, 0.0965 mmol) and K₃PO₄ (819 mg, 3.86 mmol,) were suspended in THF (30 mL) and water (3.0 mL). The reaction mixture was then purged with N₂ for 5 min and heated to 70° C. for 1 h. The reaction was cooled to room temperature, diluted with EtOAc then washed with NaHCO₃ (sat. aq.) and brine. The organics were dried over MgSO₄ and concentrated in vacuo. The residue was purified by column chromatography on silica gel, eluting with 0-100% EtOAc in cyclohexane to give the title compound (110 mg, 17%).

ESI-MS (M+H)+: 327.1, 329.0, ¹H NMR (400 MHz, DMSO) δ 9.08 (d, J=2.3 Hz, 1H), 8.78 (dd, J=0.8, 2.2 Hz, 1H), 8.75 (d, J=5.1 Hz, 1H), 8.50 (d, J=8.8 Hz, 1H), 8.33 (d, J=8.8 Hz, 1H), 8.13 (d, J=16.1 Hz, 1H), 7.85 (d, J=15.7 Hz, 1H), 7.35 (d, J=5.0 Hz, 1H), 2.55 (s, 3H).

### Synthesis of rac-7-bromo-2-((1S*,2S*)-2-(4-methylpyrimidin-2-yl)cyclopropyl)-1,5-naphthyridine

NaH (60% in mineral oil, 38 mg, 0.941 mmol) was added to a stirred solution of trimethylsulfoxonium iodide (222 mg, 1.01 mmol) in DMSO (12 mL) at room temperature under a nitrogen environment, the reaction was stirred at room temperature for 1.5 h. (E)-7-bromo-2-(2-(4-methylpyrimidin-2-yl)vinyl)-1,5-naphthyridine (110 mg, 0.336 mmol) in DMSO (25 mL) was then added and the mixture was stirred at room temperature for 18 h. The reaction was quenched with NH₄Cl (sat. aq.) and extracted with EtOAc. The organic phase was washed with brine, dried over MgSO₄ and concentrated in vacuo to give the crude title compound (100 mg) which was carried forward to next step without further purification.

ESI-MS (M+H)+: 341.1, 343.1, ¹H NMR (400 MHz, CDCl₃) δ 8.87 (d, J=2.3 Hz, 1H), 8.47-8.45 (m, 2H), 8.21 (d, J=8.4 Hz, 1H), 7.58 (d, J=8.8 Hz, 1H), 6.97 (d, J=5.1 Hz, 1H), 3.03-2.98 (m, 1H), 2.93-2.88 (m, 1H), 2.49 (s, 3H), 2.01-1.93 (m, 2H).

### Synthesis of rac-tert-butyl (6-((1S*,2S*)-2-(4-methylpyrimidin-2-yl)cyclopropyl)-1,5-naphthyridin-3-yl)carbamate

rac-7-bromo-2-((1S*,2S*)-2-(4-methylpyrimidin-2-yl)cyclopropyl)-1,5-naphthyridine (100 mg, 0.293 mmol), tert-butyl carbamate (52 mg, 0.440 mmol), Pd(OAc)₂ (3.3 mg, 0.0147 mmol), Xantphos (17 mg, 0.0293 mmol) and Cs₂CO₃ (286 mg, 0.879 mmol) were suspended in 1,4-dioxane (5.00 mL) and purged with N₂ for 5 min. The reaction was then heated to 100° C. for 1 h. The reaction was cooled to room temperature, filtered through Celite® and concentrated in vacuo. The residue was purified by column chromatography on silica gel, eluting with 0-10% MeOH in DCM to give the title compound (100 mg, 80%) of product as a yellow solid.

ESI-MS (M+H)+: 378.3, ¹H NMR (400 MHz, CDCl₃) δ 8.78 (d, J=2.5 Hz, 1H), 8.45 (d, J=5.1 Hz, 1H), 8.40-8.37 (m, 1H), 8.14 (d, J=8.9 Hz, 1H), 7.43 (d, J=9.0 Hz, 1H), 6.95 (d, J=4.9 Hz, 1H), 6.82 (s, 1H), 3.01-2.95 (m, 1H), 2.90-2.84 (m, 1H), 2.48 (s, 3H), 2.02-1.83 (m, 2H), 1.57 (s, 9H).

### Synthesis of rac-6-((1S*,2S*)-2-(4-methylpyrimidin-2-yl)cyclopropyl)-1,5-naphthyridin-3-amine

TFA (0.20 mL, 2.65 mmol) was added to a stirred solution of rac-tert-butyl (6-((1S*,2S*)-2-(4-methylpyrimidin-2-yl)cyclopropyl)-1,5-naphthyridin-3-yl)carbamate (100 mg, 0.265 mmol) in chloroform (3 mL) and stirred at room temperature for 2 h. The mixture was concentrated in vacuo and the residue was purified using an SCX cartridge, washing with 3:1 DCM in MeOH and eluting with (7N NH₃ in MeOH) in DCM to give the title compound (45 mg, 61%).

ESI-MS (M+H)+: 278.2, ¹H NMR (400 MHz, CDCl₃) δ 8.46-8.42 (m, 2H), 8.07 (d, J=8.6 Hz, 1H), 7.34 (d, J=2.5 Hz, 1H), 7.28 (s, 1H), 6.95 (d, J=5.1 Hz, 1H), 4.06 (s, 2H), 2.99-2.93 (m, 1H), 2.89-2.83 (m, 1H), 2.48 (s, 3H), 1.96-1.88 (m, 2H).

### Synthesis of rac-1-(6-cyclopropyl-2-(((6-((1S*,2S*)-2-(4-methylpyrimidin-2-yl)cyclopropyl)-1,5-naphthyridin-3-yl)amino)methyl)imidazo[1,2-a]pyridin-8-yl)-3-methylimidazolidine-2,4-dione

Ti(OiPr)₄ (0.24 mL, 0.811 mmol) was added to a stirred solution of rac-6-((1S*,2S*)-2-(4-methylpyrimidin-2-yl)cyclopropyl)-1,5-naphthyridin-3-amine (45 mg, 0.162 mmol) and 6-cyclopropyl-8-(3-methyl-2,4-dioxoimidazolidin-1-yl)imidazo[1,2-a]pyridine-2-carbaldehyde (48 mg, 0.162 mmol) in a 1:1 mixture of MeOH (2.0 mL) and DCM (2.0 mL). The mixture was then heated to 50° C. in a sealed tube for 12 h. The reaction was cooled to room temperature then NaCNBH₃ (36 mg, 0.568 mmol) was added. The mixture was then stirred at room temperature for 18 h then quenched with water (1 mL), filtered through Celite® and concentrated in vacuo. The residue was purified by silica gel column chromatography, eluting with 0-10% (7 N NH₃ in MeOH) in DCM followed by preparative HPLC to give the title compound (14.7 mg, 16%).

ESI-MS (M+H)+: 560.4, ¹H NMR (400 MHz, DMSO) δ 8.55-8.50 (m, 2H), 8.25 (s, 1H), 8.00 (d, J=8.3 Hz, 1H), 7.81 (s, 1H), 7.37-7.34 (m, 2H), 7.16 (dd, J=4.7, 4.7 Hz, 2H), 7.03 (d, J=2.3 Hz, 1H), 4.93 (s, 2H), 4.51 (d, J=5.6 Hz, 2H), 2.97 (s, 3H), 2.73 (dd, J=7.1, 7.1 Hz, 2H), 2.40 (s, 3H), 1.97-1.89 (m, 1H), 1.80-1.70 (m, 2H), 0.96-0.89 (m, 2H), 0.64 (q, J=5.2 Hz, 2H).

The compound in Table 4 was synthesized in a similar manner to rac-1-(6-cyclopropyl-2-(((6-((1S*,2S*)-2-(4-methylpyrimidin-2-yl)cyclopropyl)-1,5-naphthyridin-3-yl)amino)methyl)imidazo [1,2-a]pyridin-8-yl)-3-methylimidazolidine-2,4-dione from (E)-4-methyl-2-(2-(4,4,5,5-tetramethyl-1,3,2-dioxaborolan-2-yl)vinyl)pyrimidine and 7-bromo-2-chloro-1,6-naphthyridine.

TABLE 4

| Ex | Compound | Coupling partner | Analytical Data |
|---|---|---|---|
| 15 | 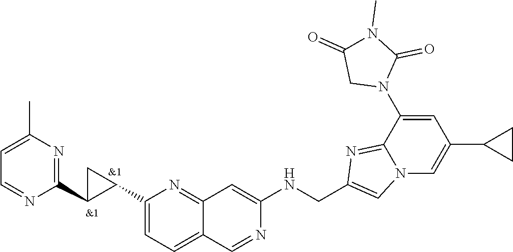 [SMILES: Cc1ccnc([C@H]2C[C@@H]2c2ccc3cnc(NCc4cn5cc(C6CC6)cc(N6CC(=O)N(C)C6=O)c5n4)cc3n2)n1] | rac-2-((1S*,2S*)- 2-(4-methyl pyrimidin-2-yl) cyclopropyl)-1,6- naphthyridin-7- amine | ESI-MS (M + H):+ 560.3, ¹H NMR (400 MHz, DMSO) δ 8.85-8.84 (m, 1H), 8.54 (d, J = 5.1 Hz, 1H), 8.45 (s, 1H), 8.25-8.24 (m, 1H), 8.12-8.08 (m, 1H), 7.74 (s, 1H), 7.35 (d, J = 1.6 Hz, 1H), 7.28 (t, J = 6.1 Hz, 1H), 7.23-7.19 (m, 2H), 6.63 (s, 1H), 4.95 (s, 2H), 4.62 (d, J = 6.0 Hz, 2H), 2.99 (s, 3H), 2.81- 2.70 (m, 2H), 2.43 (s, 3H), 1.97- 1.90 (m, 1H), 1.84-1.75 (m, 2H), 0.97-0.91 (m, 2H), 0.67-0.62 (m, 2H). |
| | I-15 | | |
| | rac-1-(6-cyclopropyl-2-(((2-((1S*,2S*)- | | |
| | 2-(4-methylpyrimidin-2-yl)cyclo- | | |
| | propyl)-1,6-naphthyridin-7-yl)amino) | | |
| | methyl)imidazo[1,2-a]pyridin-8-yl)-3- | | |
| | methylimidazolidine-2,4-dione formic | | |
| | acid salt (I-15) | | |

### Examples 16-17

### Synthesis of rac-1-(2-(((6-(2-(4-methylpyrimidin-2-yl)cyclopropyl)quinolin-3-yl)amino)methyl)imidazo[1,2-a]pyridin-8-yl)imidazolidine-2,4-dione (I-16) and rac-1-(2-((methyl(6-(2-(4-methylpyrimidin-2-yl)cyclopropyl)quinolin-3-yl)amino)methyl)imidazo[1,2-a]pyridin-8-yl)imidazolidine-2,4-dione (I-17)

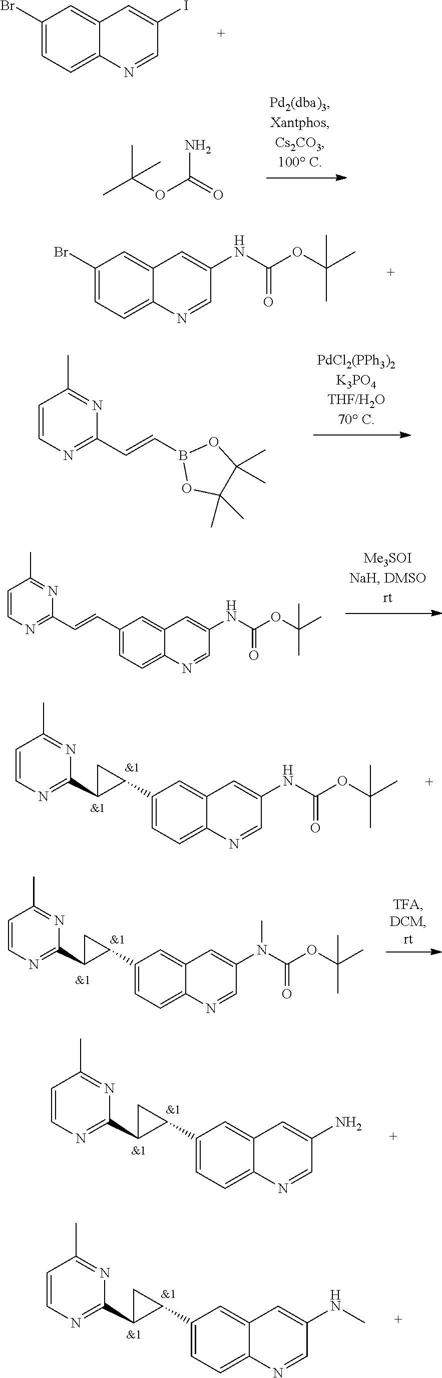

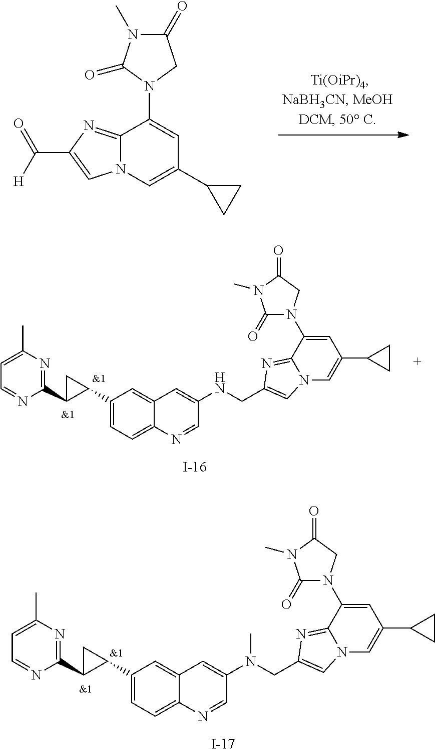

### Synthesis of tert-butyl (6-bromoquinolin-3-yl)carbamate

6-bromo-3-iodoquinoline (1.00 g, 2.99 mmol), tert-butyl carbamate (0.35 g, 2.99 mmol), Xantphos (0.69 g, 1.20 mmol), Cs₂CO₃ (2.93 g, 8.98 mmol,), Pd₂(dba)₃ (0.55 g, 0.60 mmol), in 1,4-dioxane (5.0 mL) was purged with N₂ and stirred at 100° C. overnight. The mixture was filtered through Celite® and concentrated in vacuo. The residue was purified by column chromatography on silica gel, eluting with 0-100% EtOAc in cyclohexane to give the title compound (0.55 g, 51%) as an off-white solid.

ESI-MS (M+H)+: 323.1, 325.0, ¹H NMR (400 MHz, DMSO) δ 9.98 (s, 1H), 8.88-8.86 (m, 1H), 8.48 (s, 1H), 8.21 (d, J=2.1 Hz, 1H), 7.89-7.85 (m, 1H), 7.73-7.69 (m, 1H), 1.54 (s, 9H)

### Synthesis of tert-butyl (E)-(6-(2-(4-methylpyrimidin-2-yl)vinyl)quinolin-3-yl)carbamate

tert-Butyl (6-bromoquinolin-3-yl)carbamate (540 mg, 1.67 mmol), (E)-4-methyl-2-(2-(4,4,5,5-tetramethyl-1,3,2-dioxaborolan-2-yl)vinyl)pyrimidine (411 mg, 1.67 mmol), PdCl₂(PPh₃)₂ (59 mg, 0.0835 mmol), K₃PO₄ (709 mg, 3.34 mmol,) were suspended in THF (30 mL) and H₂O (3.0 mL). The mixture was purged with N₂ and then heated to 70° C. for 18 h. The mixture was then cooled to room temperature, diluted with EtOAc and washed with NaHCO₃ (sat. aq.) then brine. The organics were passed through a hydrophobic frit and concentrated in vacuo. The residue was purified by column chromatography on silica gel, eluting with 0-100% EtOAc in cyclohexane to give the title compound (420 mg, 56%) as a pale yellow solid.

ESI-MS (M+H)+: 363.3, ¹H NMR (400 MHz, CDCl₃) 8.62-8.59 (m, 2H), 8.51-8.48 (m, 1H), 8.12 (d, J=16.1 Hz, 1H), 8.03-8.00 (m, 1H), 7.95-7.89 (m, 2H), 7.34 (d, J=15.9 Hz, 1H), 7.03-7.00 (m, 1H), 6.73 (s, 1H), 2.58 (s, 3H), 1.58 (s, 9H).

### Synthesis of rac-tert-butyl (6-((1S*,2S*)-2-(4-methylpyrimidin-2-yl)cyclopropyl)quinolin-3-yl)carbamate and rac-tert-butyl methyl(6-((1S*,2S*)-2-(4-methylpyrimidin-2-yl)cyclopropyl)quinolin-3-yl)carbamate

NaH (60% in mineral oil, 62 mg, 1.55 mmol) was added to a stirred solution of trimethylsulfoxonium iodide (364 mg, 1.66 mmol) in DMSO (5.0 mL) at room temperature for 1.5 h. tert-Butyl (E)-(6-(2-(4-methylpyrimidin-2-yl)vinyl)quinolin-3-yl)carbamate (200 mg, 0.552 mmol) in DMSO (5.0 mL) was added and the mixture was stirred at room temperature for 18 h. To the reaction mixture, a mixture of sodium hydride (60% in mineral oil, 22 mg, 0.552 mmol), trimethylsulfoxonium iodide (121 mg, 0.552 mmol) in DMSO (5.0 mL) (pre-stirred for 1 h at room temperature) was added and resulting mixture was heated to 60° C. for 3 h. The mixture was quenched NH₄Cl (sat. aq.) and extracted with EtOAc. The combined organics were passed through a hydrophobic frit then concentrated in vacuo. The residue was purified by silica gel column chromatograph, eluting with 0-100% EtOAc in cyclohexane to give a mixture of the title compounds (90 mg) which were used without further purification.

### Synthesis of rac-6-((1S*,2S*)-2-(4-methylpyrimidin-2-yl)cyclopropyl)quinolin-3-amine and rac-N-methyl-6-((1S*,2S*)-2-(4-methylpyrimidin-2-yl)cyclopropyl)quinolin-3-amine

TFA (0.037 mL, 0.478 mmol) was added to a mixture of rac-tert-butyl (6-((1S*,2S*)-2-(4-methylpyrimidin-2-yl)cyclopropyl)quinolin-3-yl)carbamate and rac-tert-butyl methyl (6-((1S*,2S*)-2-(4-methylpyrimidin-2-yl)cyclopropyl)quinolin-3-yl)carbamate (90 mg) in DCM (5.0 mL). The reaction was stirred at room temperature for 3 h. The mixture was quenched with NaHCO₃ (sat. aq.) and extracted with DCM. The combined organics were passed through a hydrophobic frit then concentrated in vacuo to give the mixture of title compounds (85 mg) as a brown solid which was used without further purification.

### Synthesis of rac-1-(6-cyclopropyl-2-(((6-((1S*,2S*)-2-(4-methylpyrimidin-2-yl)cyclopropyl)quinolin-3-yl)amino)methyl)imidazo[1,2-a]pyridin-8-yl)-3-methylimidazolidine-2,4-dione and rac-1-(6-cyclopropyl-2-((methyl(6-((1S*,2S*)-2-(4-methylpyrimidin-2-yl)cyclopropyl)quinolin-3-yl)amino)methyl)imidazo[1,2-a]pyridin-8-yl)-3-methylimidazolidine-2,4-dione

The crude mixture of rac-6-((1S*,2S*)-2-(4-methylpyrimidin-2-yl)cyclopropyl)quinolin-3-amine and rac-N-methyl-6-((1S*,2S*)-2-(4-methylpyrimidin-2-yl)cyclopropyl)quinolin-3-amine (90 mg, 0.310 mmol), 6-cyclopropyl-8-(3-methyl-2,4-dioxoimidazolidin-1-yl)imidazo[1,2-a]pyridine-2-carbaldehyde (111 mg, 0.372 mmol), Ti(OiPr)₄ (0.092 mL, 0.310 mmol) in DCM (0.50 mL) and MeOH (0.50 mL) were stirred at 50° C. for 18 h in a sealed tube. The reaction was cooled to room temperature and NaCNBH₃ (39 mg, 0.620 mmol) was added. The mixture was stirred at room temperature for 2 h. then diluted with water (1 mL), filtered through Celite® and concentrated in vacuo. The residue was purified by preparative HPLC to give the title compounds.

rac-1-(6-Cyclopropyl-2-(((6-((1S*,2S*)-2-(4-methylpyrimidin-2-yl)cyclopropyl)quinolin-3-yl)amino)methyl)imidazo[1,2-a]pyridin-8-yl)-3-methylimidazolidine-2,4-dione (4.8 mg, 2%) ESI-MS (M+H)+: 559.3, ¹H NMR (400 MHz, DMSO) δ 8.54-8.49 (m, 2H), 8.25 (d, J=1.1 Hz, 1H), 7.80 (s, 1H), 7.71-7.68 (m, 1H), 7.44 (d, J=1.9 Hz, 1H), 7.36 (d, J=1.5 Hz, 1H), 7.19-7.15 (m, 2H), 7.07 (d, J=2.6 Hz, 1H), 6.83 (t, J=5.8 Hz, 1H), 4.96-4.95 (m, 2H), 4.47 (d, J=5.8 Hz, 2H), 3.00-2.99 (m, 3H), 2.61-2.57 (m, 1H), 2.50-2.47 (m, 1H), 2.43-2.42 (m, 3H), 1.98-1.90 (m, 1H), 1.77-1.71 (m, 1H), 1.65-1.59 (m, 1H), 0.97-0.91 (m, 2H), 0.67-0.62 (m, 2H).

rac-1-(6-Cyclopropyl-2-((methyl(6-((1S*,2S*)-2-(4-methylpyrimidin-2-yl)cyclopropyl)quinolin-3-yl)amino)methyl)imidazo[1,2-a]pyridin-8-yl)-3-methylimidazolidine-2,4-dione (3.7 mg, 2%)

ESI-MS (M+H)+: 573.4, ¹H NMR (400 MHz, DMSO) δ 8.78 (d, J=3.0 Hz, 1H), 8.55 (s, 1H), 8.19 (d, J=1.0 Hz, 1H), 7.74-7.70 (m, 2H), 7.53 (d, J=1.9 Hz, 1H), 7.37 (d, J=1.5 Hz, 1H), 7.30 (d, J=2.6 Hz, 1H), 7.25-7.18 (m, 2H), 4.89 (s, 2H), 4.82 (s, 2H), 3.19 (s, 3H), 2.97 (s, 3H), 2.64-2.58 (m, 1H), 2.43 (s, 3H), 1.95-1.88 (m, 1H), 1.79-1.73 (m, 1H), 1.66-1.61 (m, 1H), 0.95-0.89 (m, 2H), 0.65-0.60 (m, 2H), cPr CH signal obscured by DMSO signal.

### Example 18

### Synthesis of 1-(2-(((2-((1S,2S)-2-(3-chlorophenyl)cyclopropyl)-4-methoxy-1,6-naphthyridin-7-yl)amino)methyl)-6-cyclopropylimidazo[1,2-a]pyridin-8-yl)-3-methylimidazolidine-2,4-dione (I-18)

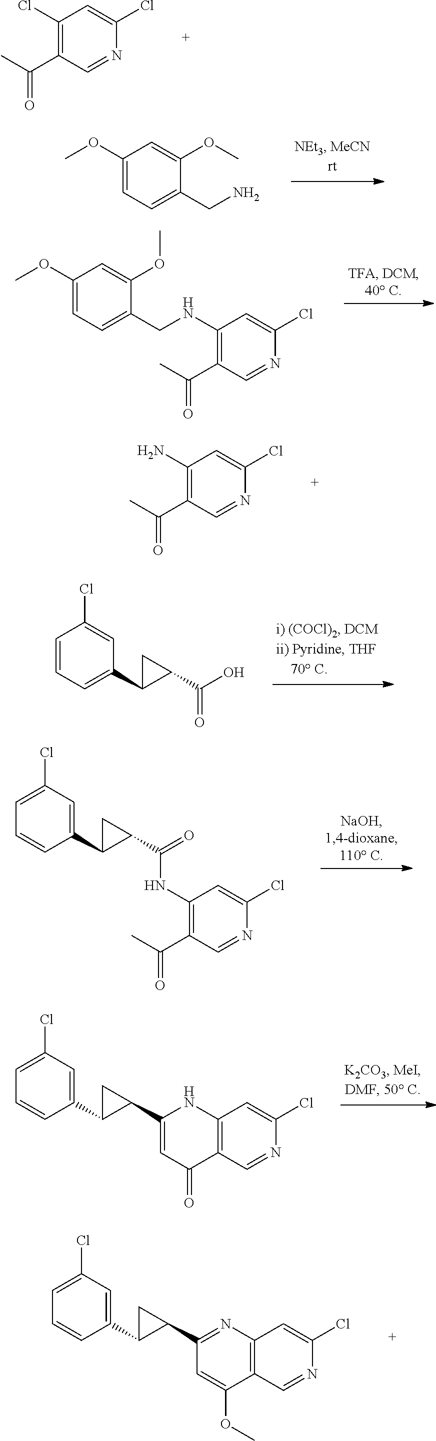

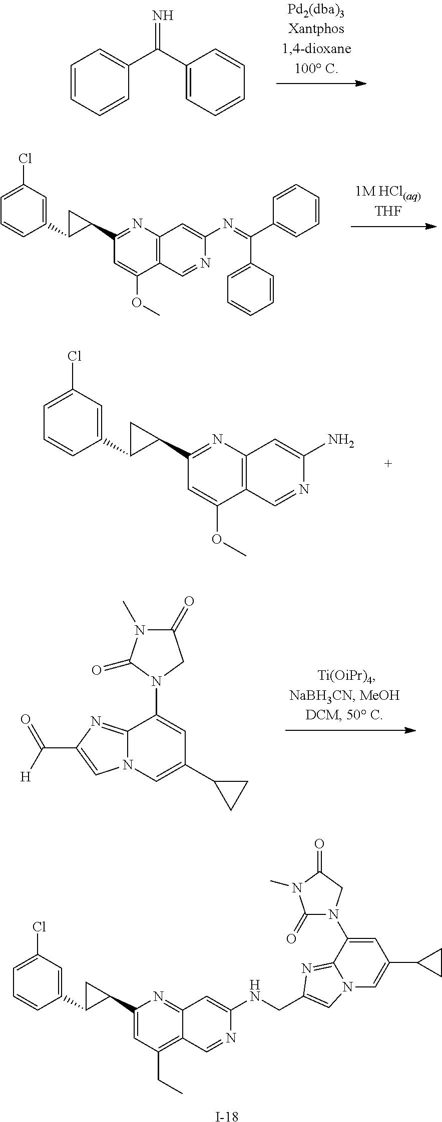

### Synthesis of 1-(6-chloro-4-((2,4-dimethoxybenzyl)amino)pyridin-3-yl)ethan-1-one

To a cooled mixture of 1-(4,6-dichloropyridin-3-yl)ethan-1-one (400 mg, 2.11 mmol) and NEt₃ (0.44 mL, 3.16 mmol) in MeCN (10 mL) at 0° C. was added (2,4-dimethoxyphenyl)methanamine (0.47 mL, 3.16 mmol) dropwise. The mixture was allowed to warm to room temperature and stirred for 18 h. Water was added and the mixture was extracted with EtOAc. The combined organics were passed through a hydrophobic frit then concentrated in vacuo to give the title compound (420 mg, 62%).

ESI-MS (M+H)+: 321, ¹H NMR (400 MHz, CDCl₃) δ 9.47-9.44 (m, 1H), 8.58 (s, 1H), 7.10 (d, J=8.3 Hz, 1H), 6.66 (s, 1H), 6.49 (d, J=2.3 Hz, 1H), 6.44 (dd, J=2.5, 8.3 Hz, 1H), 4.34 (d, J=5.8 Hz, 2H), 3.86 (s, 3H), 3.80 (s, 3H), 2.57 (s, 3H).

### Synthesis of 1-(4-amino-6-chloropyridin-3-yl)ethan-1-one

TFA (1.0 mL, 1.31 mmol) was added to a solution of 1-(6-chloro-4-((2,4-dimethoxybenzyl)amino)pyridin-3-yl)ethan-1-one (420 mg, 1.31 mmol) in DCM (20 mL) and the mixture was stirred at room temperature for 18 h. NaHCO₃ (sat. aq.) was added and the mixture was extracted with DCM. The combined organics were passed through a hydrophobic frit then concentrated in vacuo to give the title compound (260 mg, quant.).

¹H NMR (400 MHz, DMSO) δ, 8.69 (s, 1H), 6.79 (s, 1H), 2.59 (s, 3H). 2H (NIH2) missing.

### Synthesis of (1S,2S)—N-(5-acetyl-2-chloropyridin-4-yl)-2-(3-chlorophenyl)cyclopropane-1-carboxamide

Oxalyl chloride (0.44 mL, 5.09 mmol) was added dropwise to a cooled stirred solution of (1S,2S)-2-(4-methylpyrimidin-2-yl)cyclopropanecarboxylic acid (1.00 g, 5.09 mmol) and DMF (0.050 mL) in THF (25 mL) at 0° C. and stirred at 0° C. for 30 min. The reaction was then concentrated in vacuo. The residue was suspended in THF (20 mL), 1-(2-amino-4-bromo-phenyl)ethanone (87 mg, 5.09 mmol) and pyridine (1.2 mL, 15.26 mmol) were added, then the mixture was heated to 70° C. for 18 h. The mixture was cooled to room temperature and quenched with water (50 mL). The resulting precipitate was filtered, washed with water, dried to give the title compound (860 mg, 48%).

¹H NMR (400 MHz, DMSO) δ 11.74 (s, 1H), 9.00 (s, 1H), 8.46 (s, 1H), 7.33-7.20 (m, 4H), 2.67 (s, 3H), 2.56-2.53 (m, 1H), 2.35-2.29 (m, 1H), 1.60-1.48 (m, 2H).

### Synthesis of 7-chloro-2-((1S,2S)-2-(3-chlorophenyl)cyclopropyl)-1,6-naphthyridin-4 (1H)-one

NaOH (229 mg, 5.73 mmol) (which had been azetroped with toluene three times) was added to stirred suspension of (1S,2S)—N-(5-acetyl-2-chloropyridin-4-yl)-2-(3-chlorophenyl)cyclopropane-1-carboxamide (500 mg, 1.43 mmol) in 1,4-dioxane (5 mL). The reaction was heated at 100° C. for 1 h. The reaction was cooled to room temperature and poured into NH₄Cl (sat. aq., 50 mL). The resulting precipitate was filtered and dried to give the title compound (450 mg, 94%).

¹H NMR (400 MHz, DMSO) δ 11.90 (s, 1H), 8.93 (s, 1H), 7.47 (s, 1H), 7.37-7.32 (m, 2H), 7.29-7.21 (m, 2H), 6.02 (s, 1H), 2.57-2.54 (m, 1H), 2.33-2.21 (m, 1H), 1.83-1.76 (m, 1H), 1.72-1.66 (m, 1H).

### Synthesis of 7-chloro-2-((1S,2S)-2-(3-chlorophenyl)cyclopropyl)-4-methoxy-1,6-naphthyridine

MeI (0.093 mL, 1.49 mmol) was added to a stirred mixture of 7-chloro-2-((1S,2S)-2-(3-chlorophenyl)cyclopropyl)-1,6-naphthyridin-4 (1H)-one (450 mg, 1.36 mmol) and K₂CO₃ (376 mg, 2.71 mmol) in DMF (5.0 mL). The reaction was heated to 50° C. for 1 h. The reaction was cooled to room temperature and diluted with water then extracted with EtOAc. The combined organics were dried over MgSO₄ and concentrated in vacuo to give the title compound (185 mg, 39%) which was used in the next step without further purification ¹H NMR (400 MHz, DMSO) δ 9.22 (s, 1H), 7.80 (s, 1H), 7.37-7.23 (m, 5H), 4.10 (s, 3H), 2.75-2.64 (m, 2H), 2.01-1.93 (m, 1H), 1.75-1.69 (m, 1H).

### Synthesis of N-(2-((1S,2S)-2-(3-chlorophenyl)cyclopropyl)-4-methoxy-1,6-naphthyridin-7-yl)-1,1-diphenylmethanimine

Xantphos (124 mg, 0.214 mmol), benzophenone imine (0.099 mL, 0.589 mmol), Cs₂CO₃ (524 mg, 1.61 mmol) and Pd₂(dba)₃ (98 mg, 0.107 mmol) were added to solution of 7-chloro-2-((1S,2S)-2-(3-chlorophenyl)cyclopropyl)-4-methoxy-1,6-naphthyridine (185 mg, 0.536 mmol) in 1,4-dioxane (5 mL). The solution was degassed with N₂ and heated at 100° C. for 18 h. The mixture was allowed to cool to room temperature, filtered through Celite® and concentrated in vacuo. The residue was purified by silica gel column chromatography, eluting with 0-50% EtOAc in cyclohexane to give the title compound (60 mg 21%) as an off white solid.

ESI-MS (M+H)+: 490.3, ¹H NMR (400 MHz, CDCl₃) 9.20 (s, 1H), 7.85-7.80 (m, 2H), 7.51-7.38 (m, 4H), 7.07-7.03 (m, 8H), 6.94-6.94 (m, 1H), 6.54 (s, 1H), 4.00-3.99 (m, 3H), 2.65-2.59 (m, 1H), 2.33-2.28 (m, 1H), 1.97-1.92 (m, 1H), 1.54-1.48 (m, 1H).

### Synthesis of 2-((1S,2S)-2-(3-chlorophenyl)cyclopropyl)-4-methoxy-1,6-naphthyridin-7-amine

HCl (1 M aq., 1.2 mL, 1.22 mmol) was added to a stirred solution of N-(2-((1S,2S)-2-(3-chlorophenyl)cyclopropyl)-4-methoxy-1,6-naphthyridin-7-yl)-1,1-diphenylmethanimine (60 mg, 0.122 mmol) in THF (1 mL), and the reaction was stirred at 0° C. for 30 min. The reaction mixture quenched with NaHCO₃ (aq. sat.) and extracted with DCM. The organics were passed through a hydrophobic frit and concentrated in vacuo. The residue was purified by silica gel column chromatography, eluting with 0-10% (7 N NH₃ in MeOH) in DCM to give the title compound (40 mg quant.).

ESI-MS (M+H)+: 326.1 ¹H NMR (400 MHz, CDCl₃) δ 9.05 (s, 1H), 7.09-7.05 (m, 4H), 6.74-6.74 (m, 1H), 6.44 (s, 1H), 4.56 (s, 2H), 4.01 (s, 3H), 2.67-2.62 (m, 1H), 2.33-2.27 (m, 1H), 2.00-1.95 (m, 1H), 1.53-1.49 (m, 1H).

### Synthesis of 1-(2-(((2-((1S,2S)-2-(3-chlorophenyl)cyclopropyl)-4-methoxy-1,6-naphthyridin-7-yl)amino)methyl)-6-cyclopropylimidazo[1,2-a]pyridin-8-yl)-3-methylimidazolidine-2,4-dione

Ti(OiPr)₄ (0.036 mL, 0.123 mmol) was added to a stirred solution of 2-((1S,2S)-2-(3-chlorophenyl)cyclopropyl)-4-methoxy-1,6-naphthyridin-7-amine (40 mg, 0.123 mmol) and 6-cyclopropyl-8-(3-methyl-2,4-dioxoimidazolidin-1-yl)imidazo[1,2-a]pyridine-2-carbaldehyde (44 mg, 0.147 mmol), in DCM (0.50 mL) and MeOH (0.50 mL). The mixture was stirred at 50° C. in a sealed tube for 16 h then cooled to room temperature. NaCNBH₃ (15 mg, 0.246 mmol) was added and the mixture was stirred at room temperature for 2 h. The reaction was diluted with water (1.0 mL) and filtered through Celite® then concentrated in vacuo. The residue was purified by preparative HPLC gave the title compound (6.5 mg, 9%)

ESI-MS (M+H)+: 608.2, ¹H NMR (400 MHz, DMSO) δ 8.93 (s, 1H), 8.25-8.24 (m, 1H), 7.72 (s, 1H), 7.36-7.19 (m, 6H), 6.80 (s, 1H), 6.54 (s, 1H), 4.94 (s, 2H), 4.59 (d, J=5.9 Hz, 2H), 4.00 (s, 3H), 2.99 (s, 3H), 2.59-2.55 (m, 1H), 1.96-1.90 (m, 1H), 1.87-1.81 (m, 1H), 1.58-1.52 (m, 1H), 0.97-0.91 (m, 2H), 0.67-0.62 (m, 2H).

### Example 19

### Synthesis of rac-N-((6-cyclopropylimidazo[1,2-a]pyridin-2-yl)methyl)-2-((1S*,2S*)-2-(4-methylpyrimidin-2-yl)cyclopropyl)quinolin-7-amine (I-19)

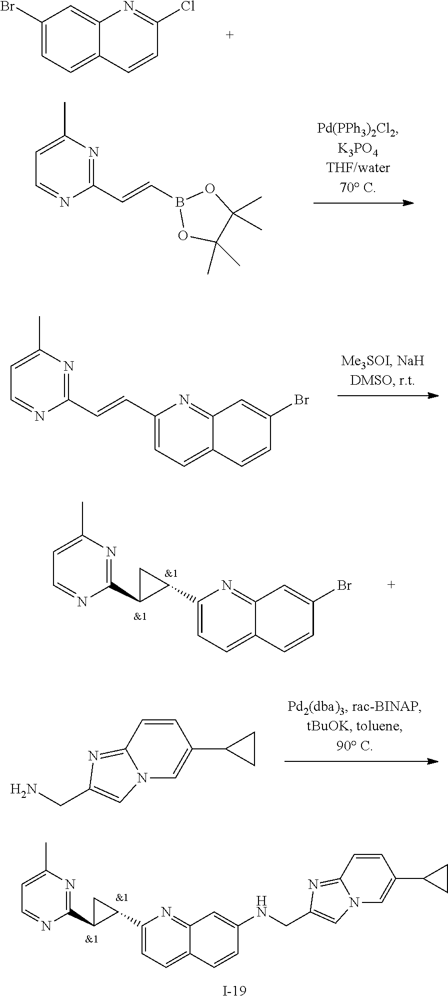

### Synthesis of (E)-7-bromo-2-(2-(4-methylpyrimidin-2-yl)vinyl)quinoline

A mixture of (E)-4-methyl-2-(2-(4,4,5,5-tetramethyl-1,3,2-dioxaborolan-2-yl)vinyl)pyrimidine (500 mg, 2.0 mmol), 7-bromo-2-chloroquinoline (490 mg, 2.0 mmol), Pd(PPh₃)₂Cl₂ (71 mg, 0.10 mmol) and K₃PO₄ (860 mg, 4.1 mmol) in THF (20 mL) and water (2.0 mL) was degassed with N₂ for 5 min and stirred at 70° C. for 3 h. The reaction mixture was cooled to room temperature, diluted with EtOAc (100 mL) then washed with NaHCO₃ (sat. aq., 100 mL) and brine (sat. aq., 150 mL). The combined organic layers were dried over MgSO₄ and concentrated in vacuo. The residue was purified by silica gel column chromatography, eluting with a gradient of 0-100% EtOAc in cyclohexane to give the title compound (300 mg, 45%).

ESI-MS (M+H)+: 326.0, 327.9, ¹H NMR (400 MHz, DMSO) δ 8.72 (d, J=5.1 Hz, 1H), 8.45 (d, J=8.6 Hz, 1H), 8.25 (d, J=1.8 Hz, 1H), 8.10-8.05 (m, 2H), 7.97 (d, J=8.8 Hz, 1H), 7.81-7.74 (m, 2H), 7.31 (d, J=5.1 Hz, 1H), 2.53 (s, 3H).

### Synthesis of rac-7-bromo-2-((1S*,2S*)-2-(4-methylpyrimidin-2-yl)cyclopropyl)quinoline

To a solution of trimethylsulfoxonium iodide (200 mg, 0.92 mmol) in DMSO (2.5 mL) was added NaH (60% in mineral oil, 34 mg, 0.86 mmol) and the reaction mixture was stirred at room temperature for 1.5 h. A mixture of (E)-7-bromo-2-(2-(4-methylpyrimidin-2-yl)vinyl)quinoline (100 mg, 0.31 mmol) in DMSO (2.5 mL) was heated until dissolution and added to the reaction mixture. The mixture was stirred at room temperature for 16 h. The reaction mixture was quenched with NH₄Cl (sat. aq., 50 mL) then diluted with EtOAc (20 mL). The mixture was extracted with EtOAc (3×50 mL) and the combined organic layers were dried over MgSO₄ and concentrated in vacuo. The residue was purified by column chromatography on silica gel, eluting with a gradient of 0-100% EtOAc in cyclohexane to give the title compound (35 mg, 34%).

ESI-MS (M+H)+: 340.0, 341.9, ¹H NMR (400 MHz, CDCl₃) δ 8.45 (d, J=5.1 Hz, 1H), 8.16 (d, J=2.0 Hz, 1H), 7.97 (d, J=8.6 Hz, 1H), 7.60 (d, J=8.8 Hz, 1H), 7.52 (dd, J=2.0, 8.6 Hz, 1H), 7.32 (d, J=8.6 Hz, 1H), 6.95 (d, J=5.1 Hz, 1H), 3.01-2.95 (m, 1H), 2.90-2.84 (m, 1H), 2.48 (s, 3H), 2.01-1.89 (m, 2H).

### Synthesis of rac-N-((6-cyclopropylimidazo[1,2-a]pyridin-2-yl)methyl)-2-((1S*,2S*)-2-(4-methylpyrimidin-2-yl)cyclopropyl)quinolin-7-amine

A mixture of rac-7-bromo-2-((1S*,2S*)-2-(4-methylpyrimidin-2-yl)cyclopropyl)quinoline (35 mg, 0.10 mmol), (6-cyclopropylimidazo[1,2-a]pyridin-2-yl)methanamine (23 mg, 0.12 mmol), rac-BINAP (6.4 mg, 0.01 mmol), Pd₂(dba)₃ (4.7 mg, 0.005 mmol) and KOtBu (23 mg, 0.21 mmol) in toluene (3.0 mL) was degassed with N₂ for 5 min and stirred at 90° C. for 4 h. The reaction mixture was cooled to room temperature, filtered through Celite® and concentrated in vacuo. The residue was purified by silica gel column chromatography, eluting with a gradient of 0-20% (7N NH₃ in MeOH) in DCM then by preparative HPLC to give the title compound (13 mg, 28%).

ESI-MS (M+H)+: 447.4, ¹H NMR (400 MHz, DMSO) δ 8.53 (d, J=5.1 Hz, 1H), 8.33-8.31 (m, 1H), 7.92 (d, J=8.2 Hz, 1H), 7.71 (s, 1H), 7.58 (d, J=8.9 Hz, 1H), 7.42 (d, J=9.4 Hz, 1H), 7.19 (d, J=5.0 Hz, 1H), 7.12 (d, J=8.3 Hz, 1H), 7.06 (dd, J=2.3, 8.9 Hz, 1H), 6.99 (dd, J=1.8, 9.3 Hz, 1H), 6.80-6.76 (m, 2H), 4.48 (d, J=5.8 Hz, 2H), 2.76-2.65 (m, 2H), 2.42 (s, 3H), 1.95-1.88 (m, 1H), 1.80-1.70 (m, 2H), 0.94-0.88 (m, 2H), 0.70-0.65 (m, 2H).

### Example 20

### Synthesis of rac-N-((6-cyclopropylimidazo[1,2-a]pyridin-2-yl)methyl)-4-methoxy-2-((1S*,2S*)-2-(4-methylpyrimidin-2-yl)cyclopropyl)quinolin-7-amine (I-20)

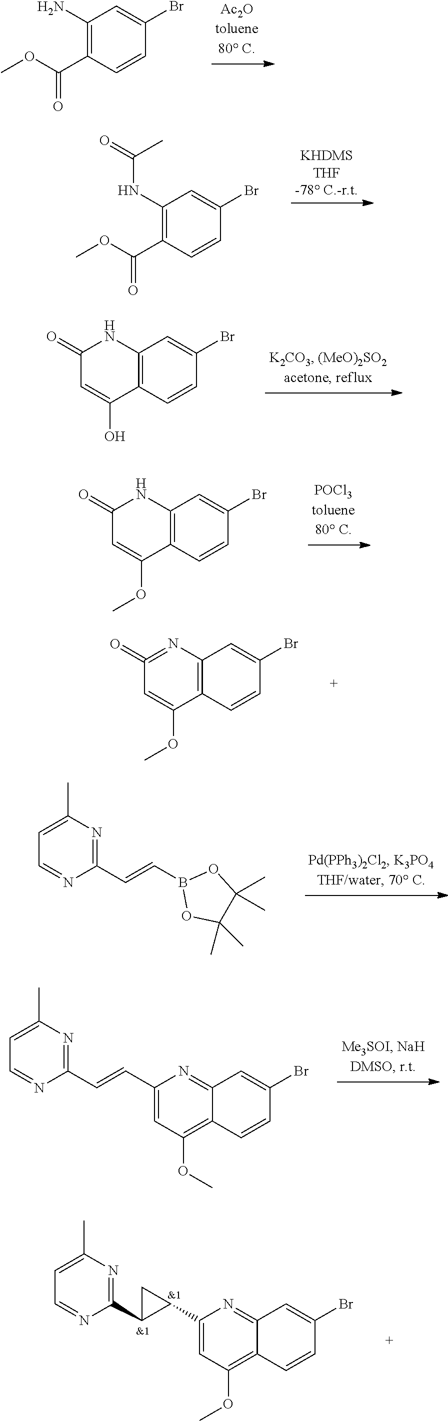

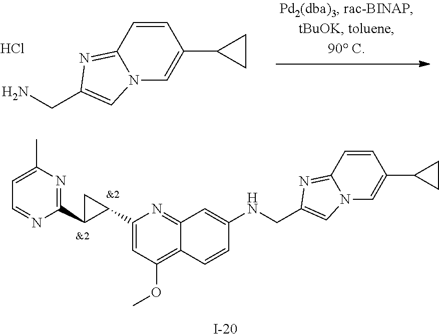

### Synthesis of methyl 2-acetamido-4-bromobenzoate

Ac₂O (8.0 mL, 85 mmol) was added to a solution of methyl 2-amino-4-bromobenzoate (13 g, 56 mmol) in toluene (150 mL) and the mixture was stirred at 80° C. for 18 h. The mixture was cooled to room temperature and concentrated in vacuo. The residue was washed with cyclohexane and EtOAc (10:1) and concentrated in vacuo to give the title compound (13 g, 85%).

ESI-MS (M+H)+: 274.0, ¹H NMR (400 MHz, CDCl₃) δ 11.06 (s, 1H), 8.97 (d, J=2.0 Hz, 1H), 7.87 (d, J=8.6 Hz, 1H), 7.21 (dd, J=1.9, 8.5 Hz, 1H), 3.93 (s, 3H), 2.24 (s, 3H).

### Synthesis of 7-bromo-4-hydroxyquinolin-2 (1H)-one

A suspension of methyl 2-acetamido-4-bromobenzoate (10 g, 37 mmol) in THF (100 mL) was added to a solution of KHMDS (1.0 M in THF, 110 mL, 110 mmol) at −78° C. The reaction mixture was stirred at −78° C. for 1 h then allowed to warm to room temperature. The reaction mixture was quenched with water (150 mL) and washed with EtOAc (200 mL). The aqueous phase was acidified with HCl (2.0 M aq.) until a precipitate formed. The precipitate was collected by filtration and washed with EtOAc then dried under high vacuum for 24 h to give the title compound (4.6 g, 52%).

ESI-MS (M+H)+: 240.0, 242.0, ¹H NMR (400 MHz, DMSO) δ 11.00-10.95 (m, 1H), 7.75 (d, J=8.3 Hz, 1H), 7.44 (d, J=1.8 Hz, 1H), 7.28 (dd, J=1.9, 8.5 Hz, 1H), 5.58 (s, 1H).

### Synthesis of 7-bromo-4-methoxyquinolin-2 (1H)-one

A mixture of 7-bromo-4-hydroxyquinolin-2 (1H)-one (6.6 g, 27 mmol) and K₂CO₃ (7.5 g, 55 mmol) in acetone (120 mL) was stirred at reflux for 3 h. Dimethyl sulfate (2.6 mL, 27 mmol) was added and the suspension was stirred at reflux for 3 h. The reaction mixture was cooled to room temperature and quenched with water (150 mL). The reaction mixture was concentrated to remove acetone and the residual aqueous mixture was filtered. The collected precipitate was washed with water and under high vacuum for 18 h to give the title compound (5.6 g, 81%).

ESI-MS (M+H)+: 254.0, 256.0, ¹H NMR (400 MHz, DMSO) δ 11.45-11.39 (m, 1H), 7.69 (d, J=8.6 Hz, 1H), 7.46 (d, J=1.8 Hz, 1H), 7.32 (dd, J=2.0, 8.6 Hz, 1H), 5.92 (s, 1H), 3.92 (s, 3H).

### Synthesis of 7-bromo-2-chloro-4-methoxyquinoline

A mixture of 7-bromo-4-methoxyquinolin-2 (11H)-one (5.5 g, 22 mmol) in POCl₃ (10 mL, 110 mmol) was stirred at 80° C. for 2 h. The reaction mixture was diluted with toluene (10 mL) and stirred at 80° C. for 2 h. The reaction mixture was cooled to room temperature and concentrated in vacuo. The residue was washed with water (50 mL), basified with NaHCO₃ (sat. aq.) and extracted with EtOAc (3×100 mL). The combined organic layers were passed through a hydrophobic frit and concentrated in vacuo to give the title compound (5.1 g, 86%).

ESI-MS (M+H)+: 273.9, ¹H NMR (400 MHz, DMSO) δ 8.13 (d, J=1.9 Hz, 1H), 8.06 (d, J=8.9 Hz, 1H), 7.77 (dd, J=2.0, 8.9 Hz, 1H), 7.19 (s, 1H), 4.11 (s, 3H).

### Synthesis of (E)-7-bromo-4-methoxy-2-(2-(4-methylpyrimidin-2-yl)vinyl)quinoline

A mixture of (E)-4-methyl-2-(2-(4,4,5,5-tetramethyl-1,3,2-dioxaborolan-2-yl)vinyl)pyrimidine (500 mg, 2.0 mmol), 7-bromo-2-chloro-4-methoxyquinoline (550 mg, 2.0 mmol), Pd(PPh₃)₂Cl₂ (71 mg, 0.10 mmol) and K₃PO₄ (860 mg, 4.1 mmol) in THF (20 mL) and water (2.0 mL) was degassed with N₂ for 5 min and stirred at 70° C. for 8 h. The reaction mixture was cooled to room temperature, diluted with EtOAc (100 mL) and washed with NaHCO₃ (sat. aq., 100 mL). The organic layer was dried over MgSO₄ and concentrated in vacuo. The residue was purified by silica gel column chromatography, eluting with a gradient of 0-100% EtOAc in cyclohexane to give the title compound (200 mg, 28%).

ESI-MS (M+H)+: 356.1, 358.1, ¹H NMR (400 MHz, CDCl₃) δ 8.62 (d, J=5.0 Hz, 1H), 8.22 (d, J=1.6 Hz, 1H), 8.15 (d, J=16.2 Hz, 1H), 8.02 (d, J=8.8 Hz, 1H), 7.71 (d, J=15.4 Hz, 1H), 7.56 (dd, J=1.9, 8.8 Hz, 1H), 7.09 (s, 1H), 7.04 (d, J=5.0 Hz, 1H), 4.08 (s, 3H), 2.58 (s, 3H).

### Synthesis of rac-7-bromo-4-methoxy-2-((1S*,2S*)-2-(4-methylpyrimidin-2-yl)cyclopropyl)quinoline

To a solution of trimethylsulfoxonium iodide (370 mg, 1.7 mmol) in DMSO (5.0 mL) was added NaH (60% in mineral oil, 63 mg, 1.6 mmol) and the reaction mixture was stirred at room temperature for 1.5 h. A mixture of (E)-7-bromo-4-methoxy-2-(2-(4-methylpyrimidin-2-yl)vinyl)quinoline (200 mg, 0.56 mmol) in DMSO (3.0 mL) was added dropwise to the reaction mixture. The reaction mixture was stirred at room temperature for 3 h. The reaction mixture was quenched with NH₄Cl (sat. aq., 80 mL) and extracted with EtOAc (3×100 mL). The combined organic layers were washed with brine (sat. aq., 100 mL), dried over MgSO₄ and concentrated in vacuo. The residue was purified by silica gel column chromatography, eluting with a gradient of 0-100% EtOAc in cyclohexane to give the title compound (50 mg, 24%).

ESI-MS (M+H)+: 370.1, 372.1, ¹H NMR (400 MHz, CDCl₃) δ 8.45 (d, J=5.1 Hz, 1H), 8.08 (d, J=1.6 Hz, 1H), 7.96 (d, J=8.9 Hz, 1H), 7.47 (dd, J=1.9, 8.8 Hz, 1H), 6.95 (d, J=5.1 Hz, 1H), 6.67 (s, 1H), 4.00 (s, 3H), 2.98-2.93 (m, 1H), 2.87-2.79 (m, 1H), 2.48 (s, 3H), 2.00-1.86 (m, 2H).

### Synthesis of rac-N-((6-cyclopropylimidazo[1,2-a]pyridin-2-yl)methyl)-4-methoxy-2-((1S*,2S*)-2-(4-methylpyrimidin-2-yl)cyclopropyl)quinolin-7-amine formic acid salt

A mixture of rac-7-bromo-4-methoxy-2-((1S*,2S*)-2-(4-methylpyrimidin-2-yl)cyclopropyl)quinoline (50 mg, 0.14 mmol), (6-cyclopropylimidazo[1,2-a]pyridin-2-yl)methanamine hydrochloride (36 mg, 0.16 mmol), rac-BINAP (8.4 mg, 0.01 mmol), Pd₂(dba)₃ (6.2 mg, 0.008 mmol) and KOtBu (45 mg, 0.41 mmol) in toluene (3.0 mL) was degassed with N₂ for 5 min and stirred at 90° C. for 16 h. The reaction mixture was cooled to room temperature, filtered through Celite® and concentrated in vacuo. The residue was purified by silica gel column chromatography, eluting with a gradient of 0-20% (7 N NH₃ in MeOH) in DCM followed by preparative HPLC to give the title compound (5.0 mg, 8%).

ESI-MS (M+H)+: 477.3, ¹H NMR (400 MHz, DMSO) δ 8.51 (d, J=5.1 Hz, 1H), 8.45 (s, 1H), 8.30 (d, J=0.4 Hz, 1H), 7.73 (d, J=9.0 Hz, 1H), 7.68 (s, 1H), 7.40 (d, J=9.2 Hz, 1H), 7.17 (d, J=5.1 Hz, 1H), 6.99-6.94 (m, 2H), 6.73-6.69 (m, 3H), 4.44 (d, J=5.9 Hz, 2H), 3.94 (s, 3H), 2.74-2.61 (m, 2H), 2.41 (s, 3H), 1.92-1.88 (m, 1H), 1.80-1.65 (m, 2H), 0.92-0.87 (m, 2H), 0.68-0.63 (m, 2H).

### Examples 21-23

### Synthesis of rac-1-(6-cyclopropyl-2-(((4-methoxy-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (I-21)

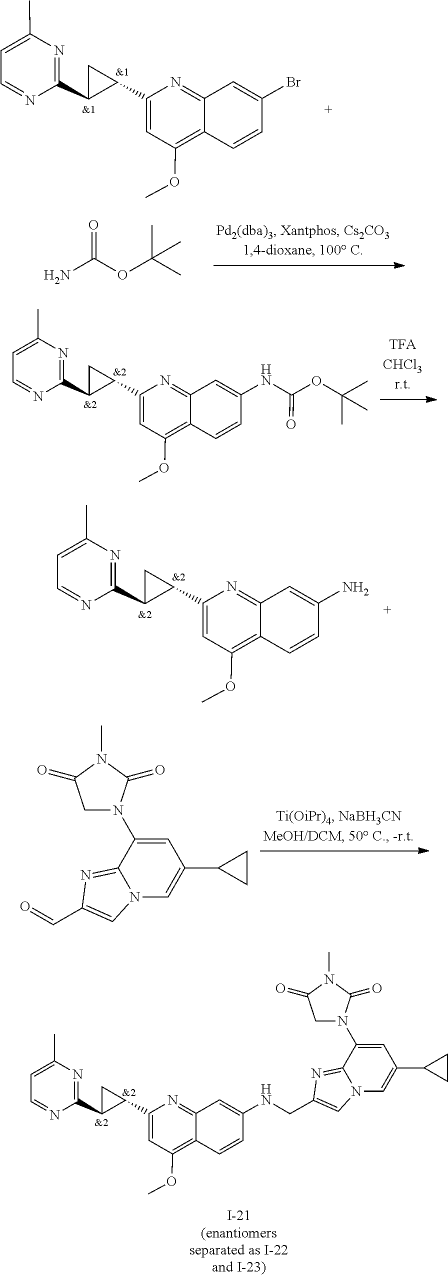

### Synthesis of rac-tert-butyl (4-methoxy-2-((1S*,2S*)-2-(4-methylpyrimidin-2-yl)cyclopropyl)quinolin-7-yl)carbamate

A suspension of rac-7-bromo-4-methoxy-2-((1S*,2S*)-2-(4-methylpyrimidin-2-yl)cyclopropyl)quinoline (70 mg, 0.19 mmol), tert-butyl carbamate (24 mg, 0.21 mmol), Xantphos (44 mg, 0.08 mmol), Cs₂CO₃ (190 mg, 0.57 mmol) in 1,4-dioxane (3.0 mL) was degassed with N₂ for 10 min. Pd₂(dba)₃ (35 mg, 0.04 mmol) was added and the reaction mixture was stirred at 100° C. for 16 h. The reaction mixture was cooled to room temperature, filtered through Celite® and concentrated in vacuo. The residue was purified by column chromatography on silica gel, eluting with a gradient of 0-100% EtOAc in cyclohexane to give the title compound (91 mg, quant.).

ESI-MS (M+H)+: 407.4, ¹H NMR (400 MHz, CDCl₃) δ 8.44 (d, J=5.0 Hz, 1H), 8.02 (d, J=8.9 Hz, 1H), 7.70 (d, J=2.1 Hz, 1H), 7.63 (d, J=9.2 Hz, 1H), 6.94 (d, J=5.0 Hz, 1H), 6.65 (s, 1H), 6.56 (s, 1H), 3.99 (s, 3H), 2.96-2.77 (m, 2H), 2.47 (s, 3H), 2.00-1.94 (m, 1H), 1.88-1.82 (m, 1H), 1.55 (s, 9H).

### Synthesis of rac-4-methoxy-2-((1S*,2S*)-2-(4-methylpyrimidin-2-yl)cyclopropyl)quinolin-7-amine

rac-tert-Butyl (4-methoxy-2-((1S*,2S*)-2-(4-methylpyrimidin-2-yl)cyclopropyl)quinolin-7-yl)carbamate (91 mg, 0.22 mmol) and TFA (0.34 mL, 4.5 mmol) was dissolved in CHCl₃ (2.0 mL) and the mixture was stirred at room temperature for 1.5 h then concentrated in vacuo. The residue was washed with NaHCO₃ (sat. aq., 25 mL) and extracted with DCM (3×40 mL). The combined organics were dried over MgSO₄ and concentrated in vacuo to give the title compound (62 mg, 90%) which was used without further purification.

ESI-MS (M+H)+: 307.2, ¹H NMR (400 MHz, CDCl₃) δ 8.44 (d, J=5.1 Hz, 1H), 7.90 (d, J=8.8 Hz, 1H), 7.05 (d, J=2.1 Hz, 1H), 6.93 (d, J=5.1 Hz, 1H), 6.82 (dd, J=2.3, 8.8 Hz, 1H), 6.43 (s, 1H), 3.95 (s, 2H), 2.94-2.77 (m, 2H), 2.47 (s, 3H), 1.95-1.82 (m, 2H).

### Synthesis of rac-1-(6-cyclopropyl-2-(((4-methoxy-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione

Ti(iPrO)₄ (0.30 mL, 1.0 mmol) was added to a solution of rac-4-methoxy-2-((1S*,2S*)-2-(4-methylpyrimidin-2-yl)cyclopropyl)quinolin-7-amine (62 mg, 0.20 mmol) and 6-cyclopropyl-8-(3-methyl-2,4-dioxoimidazolidin-1-yl)imidazo[1,2-a]pyridine-2-carbaldehyde (60 mg, 0.20 mmol) in DCM (3.0 mL) and MeOH (3.0 mL) and the reaction mixture was stirred in a sealed vial at 50° C. for 16 h. The reaction mixture was cooled to room temperature, diluted with MeOH (1.0 mL) and NaCNBH₃ was added (45 mg, 0.71 mmol). The reaction mixture was stirred at room temperature for 40 min. The reaction mixture was quenched with water (1.0 mL), diluted with MeOH (5.0 mL), filtered through Celite® with MeOH (3×3.0 mL) and concentrated in vacuo. The residue was purified by silica gel column chromatography, eluting with a gradient of 0-10% (7N NH₃ in MeOH) in DCM then by preparative HPLC to give the title compound (41 mg, 38%) as a mixture of enantiomers.

ESI-MS (M+H)+: 589.4, ¹H NMR (400 MHz, DMSO) δ 8.53 (d, J=5.0 Hz, 1H), 8.27 (s, 1H), 7.78-7.73 (m, 2H), 7.36 (s, 1H), 7.19 (d, J=5.0 Hz, 1H), 6.98 (d, J=9.0 Hz, 1H), 6.78-6.73 (m, 3H), 4.96 (s, 2H), 4.48 (d, J=5.4 Hz, 2H), 3.96 (s, 3H), 2.99 (s, 3H), 2.74-2.70 (m, 1H), 2.68-2.63 (m, 1H), 2.42 (s, 3H), 1.97-1.91 (m, 1H), 1.81-1.77 (m, 1H), 1.71-1.67 (m, 1H), 0.96-0.92 (m, 2H), 0.66 (d, J=5.0 Hz, 2H).

The mixture was separated using SFC (YMC Cellulose-C 10×250 mm, 5 μm 55/45 MeOH (0.1% DEA)/CO₂, 15 mL/min, 120 bar, 40° C.) to give two enantiomers: 1-(6-cyclopropyl-2-(((4-methoxy-2-((1S,2S)-2-(4-methylpyrimidin-2-yl)cyclopropyl)quinolin-7-yl)amino)methyl)imidazo[1,2-a]pyridin-8-yl)-3-methylimidazolidine-2,4-dione and 1-(6-cyclopropyl-2-(((4-methoxy-2-((1R,2R)-2-(4-methylpyrimidin-2-yl)cyclopropyl)quinolin-7-yl)amino)methyl)imidazo[1,2-a]pyridin-8-yl)-3-methylimidazolidine-2,4-dione.

First Eluting Isomer (Example 22, I-22)

ESI-MS (M+H)+: 589.6, ¹H NMR (400 MHz, DMSO) δ 8.51 (d, J=5.1 Hz, 1H), 8.25 (d, J=1.1 Hz, 1H), 7.74 (d, J=8.3 Hz, 2H), 7.35 (d, J=1.5 Hz, 1H), 7.17 (d, J=5.1 Hz, 1H), 6.96 (dd, J=2.3, 9.0 Hz, 1H), 6.74-6.70 (m, 3H), 4.94 (s, 2H), 4.46 (d, J=5.4 Hz, 2H), 3.94 (s, 3H), 2.97 (s, 3H), 2.73-2.61 (m, 2H), 2.40 (s, 3H), 1.96-1.89 (m, 1H), 1.80-1.65 (m, 2H), 0.95-0.89 (m, 2H), 0.66-0.61 (m, 2H). RT=2.64 min, 100% e.e.

Second Eluting Isomer (Example 23, I-23)

ESI-MS (M+H)+: 589.5, ¹H NMR (400 MHz, DMSO) δ 8.44 (d, J=5.1 Hz, 1H), 8.18 (d, J=1.1 Hz, 1H), 7.67 (d, J=8.4 Hz, 2H), 7.28 (d, J=1.5 Hz, 1H), 7.10 (d, J=5.0 Hz, 1H), 6.90 (dd, J=2.3, 9.0 Hz, 1H), 6.67-6.64 (m, 3H), 4.88 (s, 2H), 4.39 (d, J=5.4 Hz, 2H), 3.87 (s, 3H), 2.91 (s, 3H), 2.67-2.54 (m, 2H), 2.34 (s, 3H), 1.90-1.82 (m, 1H), 1.73-1.58 (m, 2H), 0.89-0.83 (m, 2H), 0.60-0.55 (m, 2H). RT=8.46 min, 100% e.e.

### Example 24

### Synthesis of rac-1-(6-cyclopropyl-2-(((3-((1S*,2S*)-2-(4-methylpyrimidin-2-yl)cyclopropyl)quinolin-6-yl)amino)methyl)imidazo[1,2-a]pyridin-8-yl)-3-methylimidazolidine-2,4-dione (I-24)

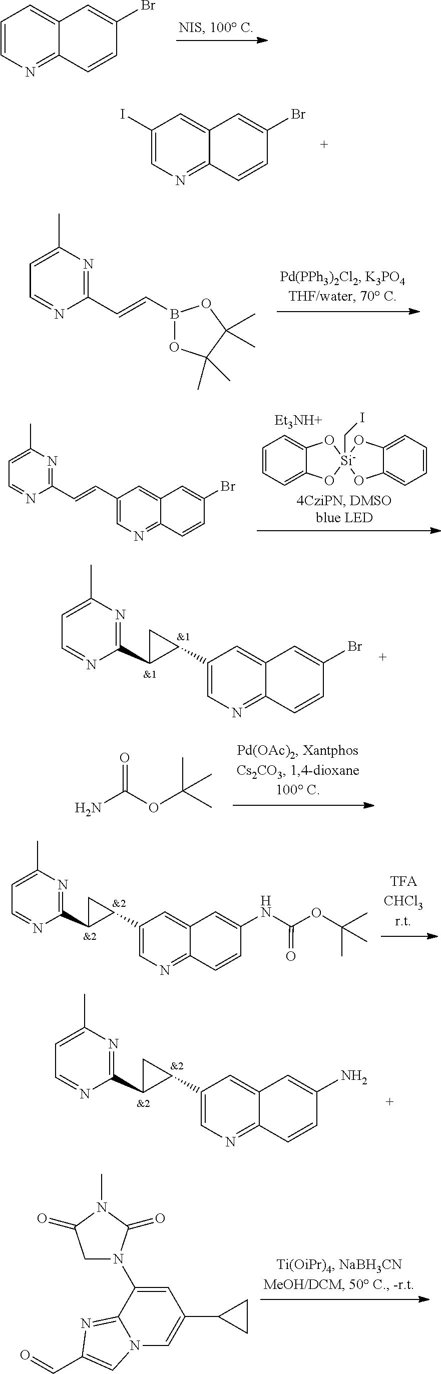

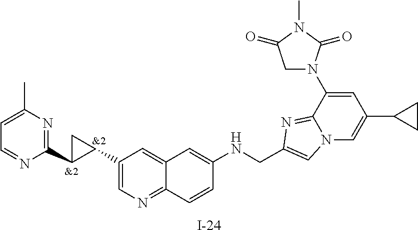

### Synthesis of 6-bromo-3-iodoquinoline

NIS (3.2 g, 14 mmol) was added portion-wise to a stirred solution of 6-bromoquinoline (2.0 g, 9.6 mmol) in AcOH (20 mL) and the reaction mixture was stirred at 100° C. for 16 h. The reaction mixture was cooled to room temperature and concentrated in vacuo. The residue was dissolved in DCM (150 mL) and washed with NaHCO₃ (sat. aq., 2×100 mL), Na₂SO₃ (sat. aq., 100 mL) and brine (sat. aq., 150 mL). The organic layer was dried over MgSO₄ and concentrated in vacuo. The residue was purified by silica gel column chromatography, eluting with a gradient of 0-100% EtOAc in cyclohexane to give the title compound (1.0 g, 31%) as an off white solid.

ESI-MS (M+H)+: 333.9, 335.9, ¹H NMR (400 MHz, CDCl₃) δ 9.04 (d, J=2.0 Hz, 1H), 8.45 (d, J=1.9 Hz, 1H), 7.93 (d, J=9.0 Hz, 1H), 7.88 (d, J=2.1 Hz, 1H), 7.81-7.77 (m, 1H).

### Synthesis of (E)-6-bromo-3-(2-(4-methylpyrimidin-2-yl)vinyl)quinoline

A suspension of 6-bromo-3-iodoquinoline (1.0 g, 3.0 mmol), (E)-4-methyl-2-(2-(4,4,5,5-tetramethyl-1,3,2-dioxaborolan-2-yl)vinyl)pyrimidine (740 mg, 3.0 mmol), Pd(PPh₃)₂Cl₂ (110 mg, 0.15 mmol) and K₃PO₄ (1300 mg, 6.0 mmol) in THF (30 mL) and water (3.0 mL) was degassed with N₂ for 5 min and the reaction mixture was stirred at 70° C. for 16 h. The reaction mixture was cooled to room temperature, diluted with EtOAc (100 mL) and washed with NaHCO₃ (sat. aq., 2×100 mL) and brine (sat. aq., 150 mL). The organic layer was dried over MgSO₄ and concentrated in vacuo. The residue was purified by silica gel column chromatography, eluting with a gradient of 0-100% EtOAc in cyclohexane to give the title compound (600 mg, 580%) as a pale yellow solid.

ESI-MS (M+H)+: 326.0, 328.0, ¹H NMR (400 MHz, DMSO) δ 9.37 (d, J=2.1 Hz, 1H), 8.73-8.68 (m, 2H), 8.29 (d, J=2.3 Hz, 1H), 8.10 (d, J=16.5 Hz, 1H), 7.99 (d, J=8.9 Hz, 1H), 7.92-7.89 (m, 1H), 7.58 (d, J=16.1 Hz, 1H), 7.30 (d, J=5.0 Hz, 1H), 2.54 (s, 3H).

### Synthesis of rac-6-bromo-3-((1S,2S*)-2-(4-methylpyrimidin-2-yl)cyclopropyl)quinolin

A suspension of (E)-6-bromo-3-(2-(4-methylpyrimidin-2-yl)vinyl)quinoline (420 mg, 1.3 mmol), triethylammonium bis(catecholato)iodomethylsilicate (1.9 g, 3.9 mmol) and 4CzIPN (100 mg, 0.13 mmol) in DMSO (30 mL) was degassed with N₂ for 5 min. The stirred mixture was irradiated with blue LEDs for 16 h. The mixture was cooled to room temperature and diluted with EtOAc (100 mL) and NaHCO₃ (sat. aq., 100 mL). The mixture was stirred, Celite® (10 g) was added and filtered to give a biphasic mixture. The layers were separated and the organic layer was washed with brine (sat. aq., 100 mL), dried over MgSO₄ and concentrated in vacuo. The residue was purified by silica gel column chromatography, eluting with a gradient of 0-100% EtOAc in cyclohexane to give the title compound (100 mg, 23%) which was used directly in the next step without further purification.

ESI-MS (M+H)+: 340.1, 342.1

### Synthesis of rac-tert-butyl (3-((1S*,2S*)-2-(4-methylpyrimidin-2-yl)cyclopropyl)quinolin-6-yl)carbamate

A mixture of rac-6-bromo-3-((1S*,2S*)-2-(4-methylpyrimidin-2-yl)cyclopropyl)quinoline (160 mg, 0.47 mmol), tert-butyl carbamate (83 mg, 0.71 mmol), Pd(OAc)₂ (5.3 mg, 0.02 mmol), Xantphos (27 mg, 0.05 mmol) and Cs₂CO₃ (460 mg, 1.4 mmol) in 1,4-dioxane (5.0 mL) was degassed with N₂ for 5 min. The reaction mixture was stirred at 100° C. for 1 h. The reaction mixture was cooled to room temperature, filtered through Celite® and concentrated in vacuo. The residue was purified by column chromatography on silica gel, eluting with a gradient of 0-10% MeOH in DCM to give the title compound (135 mg, 51%) as a yellow solid.

ESI-MS (M+H)+: 377.3

### Synthesis of rac-3-((1S*,2S*)-2-(4-methylpyrimidin-2-yl)cyclopropyl)quinolin-6-amine

TFA (0.27 mL, 3.6 mmol) was added to a stirred solution of rac-tert-butyl (3-((1S*,2S*)-2-(4-methylpyrimidin-2-yl)cyclopropyl)quinolin-6-yl)carbamate (140 mg, 0.36 mmol) in CHCl₃ (3.0 mL) and the reaction mixture was stirred at room temperature for 16 h. The reaction mixture was concentrated in vacuo and the residue was loaded onto an SCX cartridge, which was washed with MeOH in DCM (1:3) and eluted with (7N NH₃ in MeOH) in DCM (1:3) to give the title compound (75 mg, 52%).

ESI-MS (M+H)+: 277.2

### Synthesis of rac-1-(6-cyclopropyl-2-(((3-((1S*,2S*)-2-(4-methylpyrimidin-2-yl)cyclopropyl)quinolin-6-yl)amino)methyl)imidazo[1,2-a]pyridin-8-yl)-3-methylimidazolidine-2,4-dione

Ti(PrO)₄ (0.40 mL, 1.4 mmol) was added to a stirred solution of rac-3-((1S*,2S*)-2-(4-methylpyrimidin-2-yl)cyclopropyl)quinolin-6-amine (75 mg, 0.27 mmol) and 6-cyclopropyl-8-(3-methyl-2,4-dioxoimidazolidin-1-yl)imidazo[1,2-a]pyridine-2-carbaldehyde (81 mg, 0.27 mmol) in MeOH (2.0 mL) and DCM (2.0 mL). The reaction mixture was stirred at 50° C. in a sealed tube for 4 h then cooled to room temperature and NaCNBH₃ (60 mg, 0.95 mmol) was added. The reaction mixture was stirred at room temperature for 16 h then quenched with water (1.0 mL), filtered through Celite® and concentrated in vacuo. The residue was purified by silica gel column chromatography, eluting with a gradient of 0-10% (7N NH₃ in MeOH) in DCM then by preparative HPLC twice to give the title compound (17 mg, 11%) as a formic acid salt.

ESI-MS (M+H):+599.4, ¹H NMR (400 MHz, DMSO) δ 8.58 (d, J=5.1 Hz, 1H), 8.46 (d, J=1.8 Hz, 1H), 8.30 (s, 1H), 7.82 (s, 1H), 7.77-7.71 (m, 2H), 7.39 (s, 1H), 7.30-7.22 (m, 2H), 6.80-6.74 (m, 2H), 4.99 (s, 2H), 4.51 (d, J=5.6 Hz, 2H), 3.04 (s, 3H), 2.67-2.61 (m, 1H), 2.47 (s, 3H), 2.02-1.94 (m, 1H), 1.82-1.68 (m, 2H), 1.01-0.95 (m, 2H), 0.69 (q, J=5.1 Hz, 2H), 1H peak is obscured by the DMSO signal.

### Example 25

### Synthesis of rac-1-(2-(((4-(1H-imidazol-1-yl)-2-((1S*,2S*)-2-(4-methylpyrimidin-2-yl)cyclopropyl)quinolin-7-yl)amino)methyl)-6-cyclopropylimidazo[1,2-a]pyridin-8-yl)-3-methylimidazolidine-2,4-dione (I-25)

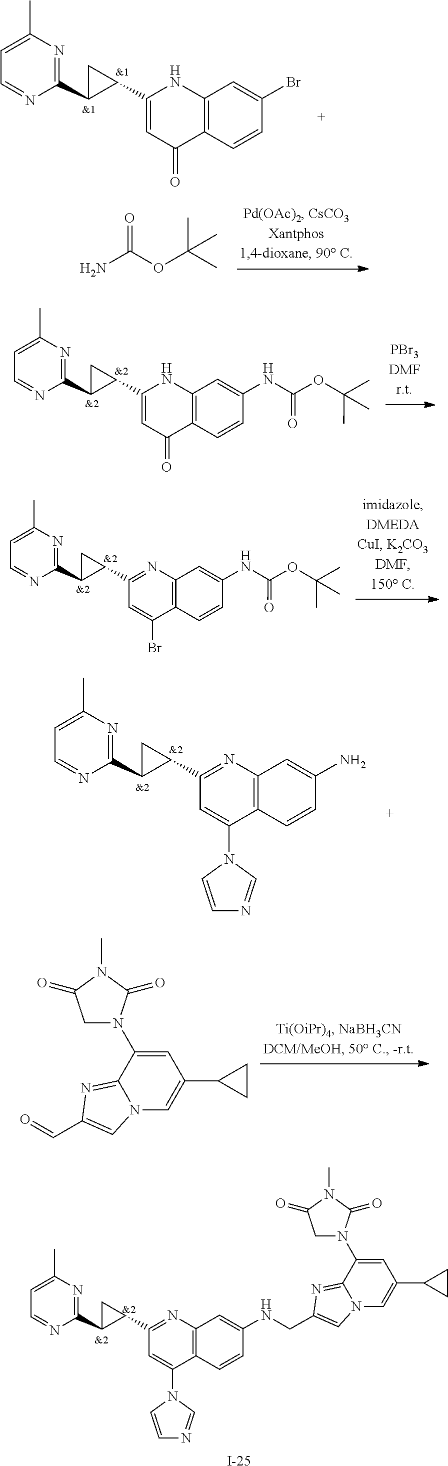

### Synthesis of rac-tert-butyl (2-((1S*,2S*)-2-(4-methylpyrimidin-2-yl)cyclopropyl)-4-oxo-1,4-dihydroquinolin-7-yl)carbamate

A mixture of rac-7-bromo-2-((1S*,2S*)-2-(4-methylpyrimidin-2-yl)cyclopropyl)quinolin-4 (11)-one (3.0 g, 8.4 mmol), tert-butyl carbamate (1.5 g, 13 mmol), Pd(OAc)₂ (95 mg, 0.42 mmol), Xantphos (490 mg, 0.84 mmol) and Cs₂CO₃ (8.2 g, 25 mmol) in 1,4-dioxane (84 mL) was degassed with N₂ for 10 min and stirred at 90° C. for 16 h. The reaction mixture was cooled to room temperature and degassed with N₂ for 10 min. Further tert-butyl carbamate (1.5 g, 13 mmol), Pd(OAc)₂ (95 mg, 0.42 mmol) and Xantphos (490 mg, 0.84 mmol) were added and the reaction mixture was stirred at 100° C. for 16 h. The mixture was cooled to room temperature and degassed with N₂. Further tert-butyl carbamate (1.5 g, 13 mmol), Pd(OAc)₂ (95 mg, 0.42 mmol), Xantphos (490 mg, 0.84 mmol) and Cs₂CO₃ (8.2 g, 25 mmol) were added and the resulting mixture was stirred at 100° C. for 16 h. The reaction mixture was cooled to room temperature, filtered through Celite® with DCM and MeOH (9:1, 100 mL) and concentrated in vacuo. The residue was purified by column chromatography on silica gel, eluting with a gradient of 0-10% MeOH in DCM to give the title compound (790 mg, 24%) as a cream solid.

ESI (M+H)+: 393.4, ¹H NMR (400 MHz, DMSO) δ 11.49 (s, 1H), 9.78 (s, 1H), 8.58-8.56 (m, 1H), 7.95-7.93 (m, 1H), 7.88 (d, J=8.8 Hz, 1H), 7.24-7.23 (m, 1H), 7.20 (q, J=3.6 Hz, 1H), 5.79 (d, J=1.8 Hz, 1H), 2.65-2.59 (m, 1H), 2.45 (s, 3H), 1.79-1.67 (m, 2H), 1.52-1.51 (m, 9H). One cyclopropyl signal obscured by DMSO signal.

### Synthesis of rac-tert-butyl (4-bromo-2-((7S*,2S*)-2-(4-methylpyrimidin-2-yl)cyclopropyl)quinolin-7-yl)carbamate

PBr₃ (0.28 mL, 3.0 mmol) was added dropwise to a mixture of rac-tert-butyl (2-((1S*,2S*)-2-(4-methylpyrimidin-2-yl)cyclopropyl)-4-oxo-1,4-dihydroquinolin-7-yl)carbamate (790 mg, 2.0 mmol) in DMF (20 mL) and the reaction mixture was placed under a N₂ atmosphere and stirred at room temperature for 16 h. The mixture was quenched with NaHCO₃ (sat. aq., 50 mL) and extracted with EtOAc (3×100 mL). The combined organic layers were dried over MgSO₄ and concentrated in vacuo. The residue was purified by column chromatography on silica gel, eluting with a gradient of 5-40% EtOAc in cyclohexane to give the title compound (280 mg, 30%).

¹H NMR (400 MHz, DMSO) δ 9.89 (s, 1H), 8.54 (d, J=5.1 Hz, 1H), 8.14 (d, J=1.8 Hz, 1H), 7.94 (d, J=9.1 Hz, 1H), 7.86 (s, 1H), 7.68 (dd, J=2.3, 9.1 Hz, 1H), 7.20 (d, J=5.6 Hz, 1H), 2.83-2.77 (m, 2H), 2.42 (s, 3H), 1.88-1.77 (m, 2H), 1.52 (s, 9H).

### Synthesis of rac-4-(1H-imidazol-1-yl)-2-((1S*,2S*)-2-(4-methylpyrimidin-2-yl)cyclopropyl)quinolin-7-amine

A mixture of rac-tert-butyl (4-bromo-2-((1S*,2S*)-2-(4-methylpyrimidin-2-yl)cyclopropyl)quinolin-7-yl)carbamate (75 mg, 0.17 mmol), 1H-imidazole (13 mg, 0.18 mmol), CuI (3.1 mg, 0.017 mmol), DMEDA (0.0035 mL, 0.033 mmol) and K₂CO₃ (46 mg, 0.33 mmol) in DMF (1.7 mL) was degassed with N₂ for 15 min and stirred at 150° C. for 12 h under a N₂ atmosphere. Further CuI (3.1 mg, 0.017 mmol) was added and the mixture was degassed with N₂ for 5 min then stirred at 150° C. for 4 h. The reaction mixture was cooled to room temperature, diluted with EtOAc (25 mL) and washed with NaHCO₃ (sat. aq., 25 mL) and brine (sat. aq., 25 mL). The combined aqueous layers were extracted with EtOAc (20 mL). The combined organic layers were dried over MgSO₄ and concentrated in vacuo to give the title compound (50 mg, 89%) as a yellow gum which was used without further purification.

ESI (M+H)+: 343.2.

### Synthesis of rac-1-(2-(((4-(1H-imidazol-1-yl)-2-((1S*,2S*)-2-(4-methylpyrimidin-2-yl)cyclopropyl)quinolin-7-yl)amino)methyl)-6-cyclopropylimidazo[1,2-a]pyridin-8-yl)-3-methylimidazolidine-2,4-dione

Ti(OiPr)₄ (0.22 mL, 0.73 mmol) was added to a solution of rac-4-(1H-imidazol-1-yl)-2-((1S*,2S*)-2-(4-methylpyrimidin-2-yl)cyclopropyl)quinolin-7-amine (50 mg, 0.15 mmol) and 6-cyclopropyl-8-(3-methyl-2,4-dioxoimidazolidin-1-yl)imidazo[1,2-a]pyridine-2-carbaldehyde (44 mg, 0.15 mmol) in MeOH (2.2 mL) and DCM (2.2 mL). The solution was sealed and stirred at 50° C. for 16 h. The reaction mixture was cooled to room temperature and NaCNBH₃ (32 mg, 0.51 mmol) was added. The mixture was stirred at room temperature for 5 h. Water (2.0 mL) was added and the mixture was filtered through Celite® then concentrated in vacuo. The residue was purified by silica gel column chromatography, eluting with a gradient of 1-20% (7N NH₃ in MeOH) in DCM then by preparative HPLC to give the title compound (8 mg, 9%) as a yellow lyophilized solid.

ESI (M+H)+: 625.4, ¹H NMR (400 MHz, DMSO) δ 8.52 (d, J=5.1 Hz, 1H), 8.27-8.25 (m, 1H), 8.06-8.05 (m, 1H), 7.77 (s, 1H), 7.62-7.61 (m, 1H), 7.41 (d, J=9.1 Hz, 1H), 7.35 (d, J=1.5 Hz, 1H), 7.22 (s, 1H), 7.20-7.11 (m, 3H), 7.06 (t, J=6.0 Hz, 1H), 6.90 (d, J=2.3 Hz, 1H), 4.94 (s, 2H), 4.52 (d, J=5.8 Hz, 2H), 2.97 (s, 3H), 2.81-2.72 (m, 2H), 2.41 (s, 3H), 1.97-1.90 (m, 1H), 1.86-1.72 (m, 2H), 0.96-0.90 (m, 2H), 0.67-0.62 (m, 2H).

### Example 26

### Synthesis of rac-1-(6-cyclopropyl-2-(((2-((1S*,2S*)-2-(4-methylpyrimidin-2-yl)cyclopropyl)-4-(1H-1,2,4-triazol-1-yl)quinolin-7-yl)amino)methyl)imidazo[1,2-a]pyridin-8-yl)-3-methylimidazolidine-2,4-dione (I-26)

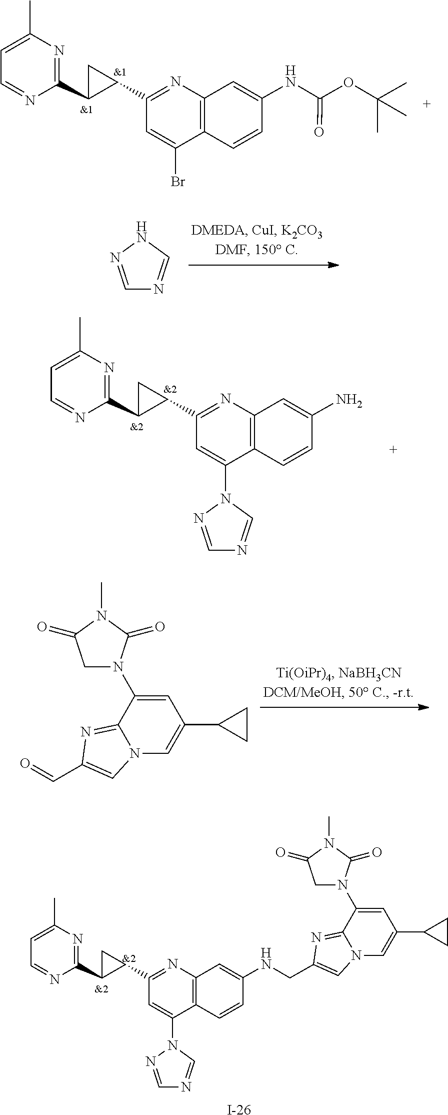

### Synthesis of rac-2-((1S*,2S*)-2-(4-methylpyrimidin-2-yl)cyclopropyl)-4-(H-1,2,4-triazol-1-yl)quinolin-7-amine

A mixture of rac-tert-butyl (4-bromo-2-((1S*,2S*)-2-(4-methylpyrimidin-2-yl)cyclopropyl)quinolin-7-yl)carbamate (63 mg, 0.14 mmol), 1,2,4-triazole (10 mg, 0.14 mmol), DEMDA (1.5 μL, 0.014 mmol), CuI (1.3 mg, 6.9 μmol), K₂CO₃ (38 mg, 0.28 mmol) in DMF (3.00 mL) was placed under N₂ atmosphere and stirred at 150° C. for 16 h. The reaction mixture was cooled to room temperature, diluted with EtOAc (50 mL) and washed with NaHCO₃ (sat. aq., 50 mL) and brine (sat. aq., 50 mL). The combined organic layers were dried over MgSO₄ and concentrated in vacuo. The residue was loaded onto an SCX cartridge, washed with MeOH in DCM (1:1), eluted with (7 N NH₃ in MeOH) in DCM (1:1) and concentrated in vacuo to give the title compound (54 mg, quant.).

ESI (M+H)+: 344.2, ¹H NMR (400 MHz, CDCl₃) δ 8.51 (s, 1H), 8.45 (d, J=5.1 Hz, 1H), 8.24-8.22 (m, 1H), 7.71 (d, J=8.8 Hz, 1H), 7.19 (d, J=2.3 Hz, 1H), 7.11-7.10 (m, 1H), 6.97-6.92 (m, 2H), 4.21-4.17 (m, 2H), 3.01-2.94 (m, 1H), 2.89-2.83 (m, 1H), 2.48 (s, 3H), 2.03-1.96 (m, 1H), 1.95-1.89 (m, 1H).

### Synthesis of rac-1-(6-cyclopropyl-2-(((2-((1S*,2S*)-2-(4-methylpyrimidin-2-yl)cyclopropyl)-4-(1H-1,2,4-triazol-1-yl)quinolin-7-yl)amino)methyl)imidazo[1,2-a]pyridin-8-yl)-3-methylimidazolidine-2,4-dione

Ti(OiPr)₄ (0.23 mL, 0.79 mmol) was added to a mixture of rac-2-((1S*,2S*)-2-(4-methylpyrimidin-2-yl)cyclopropyl)-4-(1H-1,2,4-triazol-1-yl)quinolin-7-amine (54 mg, 0.16 mmol) and 6-cyclopropyl-8-(3-methyl-2,4-dioxoimidazolidin-1-yl)imidazo[1,2-a]pyridine-2-carbaldehyde (47 mg, 0.16 mmol) in MeOH (2.5 mL) and DCM (2.5 mL). The reaction mixture was degassed with N₂ for 10 min and stirred in a sealed vial at 50° C. for 16 h. The reaction mixture was cooled to room temperature, diluted with MeOH (1.0 mL) and NaCNBH₃ (35 mg, 0.55 mmol) was added. The reaction mixture was stirred at room temperature for 4 h. Further NaCNBH₃ (35 mg, 0.55 mmol) was added and the reaction mixture was stirred at room temperature for 16 h. The reaction mixture was quenched with water (2.0 mL), filtered through a pad of Celite® and concentrated in vacuo. The residue was purified by preparative HPLC to give the title compound (19 mg, 19%) as yellow solid.

ESI (M+H)+: 626.4, ¹H NMR (400 MHz, DMSO) δ 9.14 (s, 1H), 8.52 (d, J=5.0 Hz, 1H), 8.38 (s, 1H), 8.26 (d, J=1.1 Hz, 1H), 7.77 (s, 1H), 7.73 (d, J=9.2 Hz, 1H), 7.37 (s, 1H), 7.35 (d, J=1.5 Hz, 1H), 7.18 (d, J=5.1 Hz, 1H), 7.14 (dd, J=2.2, 9.2 Hz, 1H), 7.08 (t, J=6.0 Hz, 1H), 6.91 (d, J=2.0 Hz, 1H), 4.94 (s, 2H), 4.52 (d, J=5.6 Hz, 2H), 2.97 (s, 3H), 2.81-2.74 (m, 2H), 2.41 (s, 3H), 1.95-1.90 (m, 1H), 1.87-1.83 (m, 1H), 1.79-1.74 (m, 1H), 0.96-0.90 (m, 2H), 0.66-0.62 (m, 2H).

### Example 27

### Synthesis of 1-(6-cyclopropyl-2-(((4-(1-methyl-1H-pyrazol-4-yl)-2-((1S,2S)-2-(4-methylpyrimidin-2-yl)cyclopropyl)quinolin-7-yl)amino)methyl)imidazo[1,2-a]pyridin-8-yl)-3-methylimidazolidine-2,4-dione (I-27)

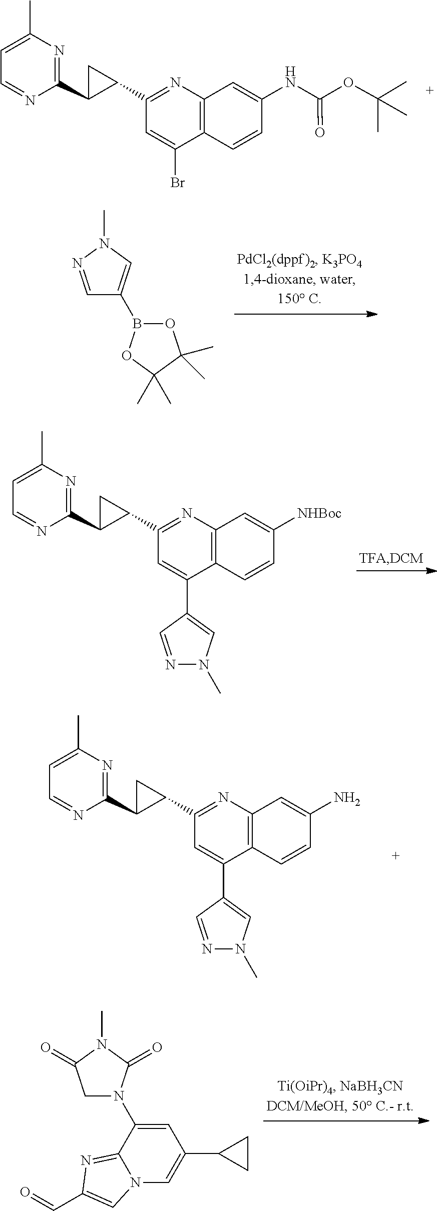 [SMILES: Cc1ccnc([C@H]2C[C@@H]2c2cc(Br)c3ccc(NC(=O)OC(C)(C)C)cc3n2)n1 ; Cn1cc(B2OC(C)(C)C(C)(C)O2)cn1 ; Cc1ccnc([C@H]2C[C@@H]2c2cc(-c3cnn(C)c3)c3ccc(NC(=O)OC(C)(C)C)cc3n2)n1 ; Cc1ccnc([C@H]2C[C@@H]2c2cc(-c3cnn(C)c3)c3ccc(N)cc3n2)n1 ; CN1C(=O)CN(c2cc(C3CC3)cn3cc(C=O)nc23)C1=O]

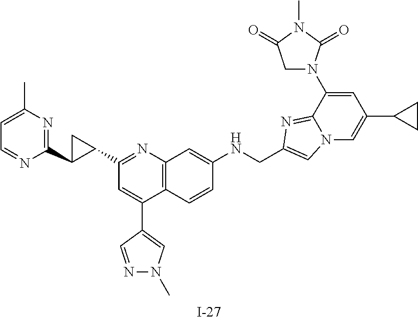

### Synthesis of tert-butyl (4-(1-methyl-1H-pyrazol-4-yl)-2-((1S,2S)-2-(4-methylpyrimidin-2-yl)cyclopropyl)quinolin-7-yl)carbamate

A mixture of tert-butyl (4-bromo-2-((1S,2S)-2-(4-methylpyrimidin-2-yl)cyclopropyl)quinolin-7-yl)carbamate (48 mg, 0.11 mmol) (prepared using the same route as rac-tert-butyl (4-bromo-2-((1S*,2S*)-2-(4-methylpyrimidin-2-yl)cyclopropyl)quinolin-7-yl)carbamate from (1S, 2S)-2-(4-methylpyrimidin-2-yl)cyclopropane-1-carboxylic acid), 1-methyl-4-(4,4,5,5-tetramethyl-1,3,2-dioxaborolan-2-yl)-1H-pyrazole (24 mg, 0.12 mmol) and K₃PO₄ (67 mg, 0.32 mmol) in dioxane (1.0 mL) and water (0.2 mL) was degassed with N₂ for 5 min. PdCl₂(dppf)₂ (4.3 mg, 0.0053 mmol) was added and the mixture heated in a microwave reactor at 100° C. for 30 min. The mixture was cooled for room temperature, filtered through Celite® then concentrated in vacuo. The residue was purified by silica gel column chromatography, eluting with a gradient of 0-100% EtOAc in cyclohexane to give the title compound (32 mg, 67%) as a golden gum.

ESI (M+H)+: 457.5

### Synthesis of 4-(1-methyl-1H-pyrazol-4-yl)-2-((1S,2S)-2-(4-methylpyrimidin-2-yl)cyclopropyl)quinolin-7-amine

tert-Butyl (4-(1-methyl-1H-pyrazol-4-yl)-2-((1S,2S)-2-(4-methylpyrimidin-2-yl)cyclo propyl)quinolin-7-yl)carbamate (32 mg, 0.070 mmol) was dissolved in DCM (2.0 mL) and TFA (0.4 mL) was added. The mixture was stirred for 90 min then concentrated in vacuo. The residue was loaded onto an SCX cartridge, washed with DCM then 10% MeOH in DCM, eluted with 10% (7N NH₃ in MeOH) in DCM then concentrated in vacuo to give the title compound (20 mg, 80%) as a yellow solid. ESI (M+H)+: 357.3

### Synthesis of 1-(6-cyclopropyl-2-(((4-(1-methyl-1H-pyrazol-4-yl)-2-((1S,2S)-2-(4-methylpyrimidin-2-yl)cyclopropyl)quinolin-7-yl)amino)methyl)imidazo[1,2-a]pyridin-8-yl)-3-methylimidazolidine-2,4-dione

Using a similar procedure to that used for rac-1-(6-cyclopropyl-2-(((2-((1S*,2S*)-2-(4-methylpyrimidin-2-yl)cyclopropyl)-4-(1H-1,2,4-triazol-1-yl)quinolin-7-yl)amino)methyl)imidazo[1,2-a]pyridin-8-yl)-3-methylimidazolidine-2,4-dione, the title compound was prepared (as a formic acid salt) from 4-(1-methyl-1H-pyrazol-4-yl)-2-((1S,2S)-2-(4-methylpyrimidin-2-yl)cyclopropyl)quinolin-7-amine (20 mg, 0.056 mmol) and 6-cyclopropyl-8-(3-methyl-2,4-dioxoimidazolidin-1-yl)imidazo[1,2-a]pyridine-2-carbaldehyde (17 mg, 0.056 mmol) then purified by preparative HPLC (19 mg, 53%).

ESI (M+H)+: 639.5, ¹H NMR (400 MHz, DMSO) δ 8.53 (d, J=5.0 Hz, 1H), 8.28-8.18 (m, 2H), 7.91-7.86 (m, 2H), 7.78 (s, 1H), 7.37 (d, J=1.5 Hz, 1H), 7.20-7.14 (m, 2H), 7.09 (dd, J=2.4, 9.2 Hz, 1H), 6.85-6.83 (m, 2H), 4.97 (s, 2H), 4.52 (d, J=5.6 Hz, 2H), 3.95 (s, 3H), 2.99 (s, 3H), 2.77-2.65 (m, 2H), 2.43 (s, 3H), 1.98-1.91 (m, 1H), 1.83-1.69 (m, 2H), 0.97-0.91 (m, 2H), 0.68-0.63 (m, 2H).

### Example 28

### Synthesis of 1-(6-cyclopropyl-2-(((5-methoxy-2-((1S,2S)-2-(4-methylpyrimidin-2-yl)cyclopropyl)quinolin-7-yl)amino)methyl)imidazo[1,2-a]pyridin-8-yl)-3-methylimidazolidine-2,4-dione (I-28)

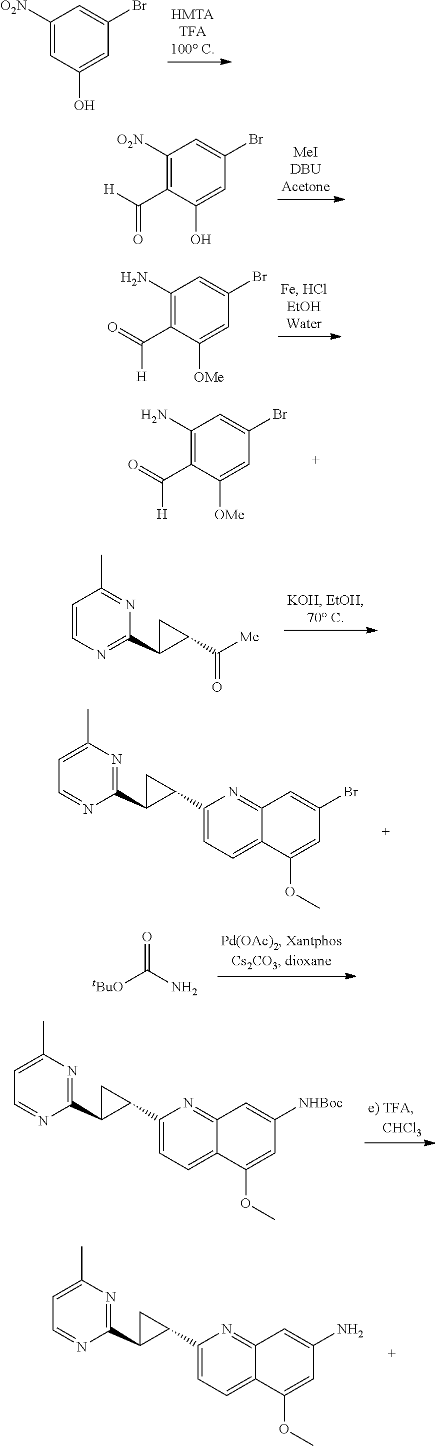

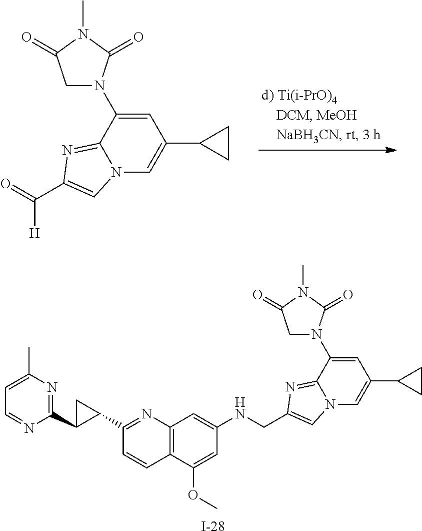

### Synthesis of 1-(6-cyclopropyl-2-(((5-methoxy-2-((1S,2S)-2-(4-methylpyrimidin-2-yl)cyclo propyl)quinolin-7-yl)amino)methyl)imidazo[1,2-a]pyridin-8-yl)-3-methylimidazolidine-2,4-dione

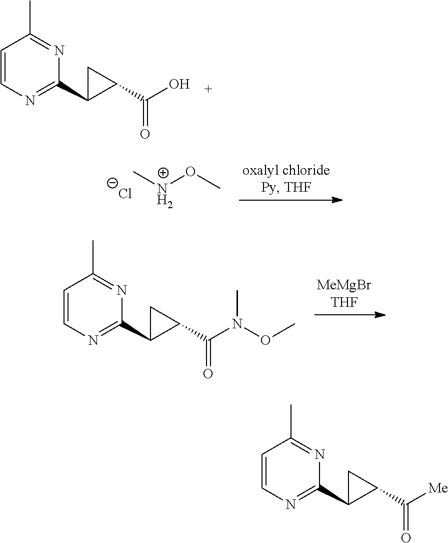

### Synthesis of (1S,2S)—N-methoxy-N-methyl-2-(4-methylpyrimidin-2-yl)cyclopropane-1-carboxamide

Oxalyl chloride (7.3 mL, 84.2 mmol) was added dropwise to a stirred solution of (1S,2S)-2-(4-methylpyrimidin-2-yl)cyclopropanecarboxylic acid (7.50 g, 42.1 mmol) and DMF (0.050 mL) in THF (10 mL) at 0° C. and stirred at 0° C. for 30 min. The reaction was then concentrated to dryness. The residue was suspended in THF (20 mL) followed by the addition of N,O-dimethylhydroxylamine hydrochloride (4.93 g, 50.5 mmol) and pyridine (14 mL, 0.168 mol). The reaction was then heated to 50° C. and stirred for 3 h then cooled to room temperature and partitioned between water and DCM. The layers were separated and the aqueous was further extracted with DCM. The combined organics were dried over MgSO₄ and concentrated. The residue was purified by silica gel column chromatography, eluting with 0-10% MeOH in DCM to give the title compound (7.4 g, 79%), as a brown oil.

ESI-MS (M+H)+: 222.3, ¹H NMR (400 MHz, CDCl₃) δ 8.43 (d, J=5.1 Hz, 1H), 6.95 (d, J=5.1 Hz, 1H), 3.71 (s, 3H), 3.26-3.21 (m, 3H), 2.81-2.71 (m, 2H), 2.47 (s, 3H), 1.73-1.66 (m, 1H), 1.64-1.58 (m, 1H).

### Synthesis of 1-((1S,2S)-2-(4-methylpyrimidin-2-yl)cyclopropyl)ethan-1-one

Methylmagnesium bromide solution (3 M in Et₂O, 20 mL, 60.2 mmol) was added dropwise over 5 min to a stirred solution of (1S,2S)—N-methoxy-N-methyl-2-(4-methylpyrimidin-2-yl)cyclopropane-1-carboxamide (7.40 g, 33.4 mmol) in THF (60 mL) at 0° C., the reaction was stirred at 0° C. for 1 h. The reaction was warmed to room temperature and quenched with NH₄Cl (sat. aq.). The mixture was then extracted with EtOAc (×3). The combined organics were dried over MgSO₄ and concentrated in vacuo. The residue was purified by silica gel column chromatography, eluting with 0-100% ethyl acetate in cyclohexane to give the title compound (4.76 g, 81%) as a blue oil.

ESI-MS (M+H)+: 177.2, ¹H NMR (400 MHz, DMSO) δ 8.50 (d, J=5.0 Hz, 1H), 7.19 (d, J=5.4 Hz, 1H), 2.58-2.52 (m, 2H), 2.40 (s, 3H), 2.24 (s, 3H), 1.57-1.48 (m, 2H).

### Synthesis of 4-bromo-2-hydroxy-6-nitrobenzaldehyde

3-bromo-5-nitro-phenol (2.0 g, 9.17 mmol) and hexamethylenetetramine (2.57 g, 18.3 mmol) were suspended in TFA (10 mL, 0.131 mol) and heated in a sealed tube at 100° C. for 24 h. The reaction was cooled to room temperature and poured into ice/water. The solution was then extracted with DCM (×3). The combined organics were dried over MgSO₄ and concentrated in vacuo. The residue was purified by silica gel column chromatography, eluting with 0-100% EtOAc in cyclohexane to give the title product as a mixture of two regioisomers (1.15 g, 51%).

ESI-MS (M+H)+: 244.0/246.0, Regioisomer A ¹H NMR (400 MHz, CDCl₃) δ 12.21 (s, 1H), 10.29 (s, 1H), 7.69 (d, J=1.8 Hz, 1H), 7.49 (d, J=1.5 Hz, 1H), Regioisomer B ¹H NMR (400 MHz, CDCl₃) δ 12.11 (s, 1H), 10.42 (s, 1H), 7.99 (d, J=2.1 Hz, 1H), 7.78 (d, J=1.8 Hz, 1H), 1.43 (s, 1H).

### Synthesis of 4-bromo-2-methoxy-6-nitrobenzaldehyde

1,8-Diazabicyclo[5.4.0]undec-7-ene (1.4 mL, 9.35 mmol) and iodomethane (0.87 mL, 14.0 mmol) were added to a stirred solution of 4-bromo-2-hydroxy-6-nitro-benzaldehyde (1.15 g, 4.67 mmol) in acetone (20 mL). The reaction was then stirred at room temperature for 16 h. The reaction was concentrated and then suspended in EtOAc and NaHCO₃. The aqueous layer was further extracted with EtOAc. The combined organics were dried over MgSO₄ and concentrated in vacuo. The crude residue was purified by silica gel column chromatography, eluting with 0-100% EtOAc in hexane to give a mixture of regioisomers (1.0 g, 82%).

Isomer A ¹H NMR (400 MHz, CDCl₃) δ 10.30 (s, 1H), 7.55 (s, 1H), 7.36 (s, 1H), 3.96 (s, 3H). Isomer B ¹H NMR (400 MHz, CDCl₃) δ 10.38 (s, 1H), 8.10 (d, J=1.8 Hz, 1H), 7.79 (d, J=1.8 Hz, 1H), 4.03 (s, 3H).

### Synthesis of 2-amino-4-bromo-6-methoxybenzaldehyde

Hydrogen chloride (2 M, 10 mL, 2.00 mmol), was added to a stirred mixture of iron (322 mg, 5.77 mmol) and 4-bromo-2-methoxy-6-nitrobenzaldehyde (500 mg, 1.92 mmol) in EtOH (10 mL) and water (2.5 mL). The reaction was then heated to 60° C. and stirred for 5 h. The reaction was cooled to room temperature and concentrated, the residue was diluted with DCM and NaHCO₃. The aqueous layer was further extracted with DCM. The combined organics were dried over MgSO₄ and concentrated in vacuo. The residue was purified by silica gel column chromatography, eluting with a gradient of 0-10% MeOH in DCM to give the title compound (160 mg, 36%).

ESI-MS (M+H)+: 230.1, 232.1, ¹H NMR (400 MHz, DMSO) δ 10.26-10.26 (m, 1H), 7.60 (s, 2H), 6.63 (d, J=1.8 Hz, 1H), 6.39 (d, J=1.5 Hz, 1H), 3.89 (s, 3H).

### Synthesis of 7-bromo-5-methoxy-2-((1S,2S)-2-(4-methylpyrimidin-2-yl)cyclopropyl)quinoline

KOH (25.4 mg, 0.45 mmol) was added to a stirred solution of 1-((1S,2S)-2-(4-methylpyrimidin-2-yl)cyclopropyl)ethan-1-one (80 mg, 0.45 mmol) and 2-amino-4-bromo-6-methoxybenzaldehyde (104 mg, 0.45 mmol) in EtOH (3.0 mL) which was then stirred at 70° C. for 30 min. The reaction was cooled to room temperature and concentrated in vacuo. The residue was dissolved in DCM and washed with NaHCO₃. The organics were dried over MgSO₄ and concentrated in vacuo. The residue was purified by silica gel column chromatography, eluting with 0-100% EtOAc in cyclohexane to give the title compound (80 mg, 48%).

ESI-MS (M+H)+: 370.2, 372.2, ¹H NMR (400 MHz, CDCl₃) δ 8.44 (d, J=5.1 Hz, 1H), 8.33 (d, J=8.3 Hz, 1H), 7.75 (s, 1H), 7.28 (d, J=8.6 Hz, 1H), 6.94 (d, J=5.1 Hz, 1H), 6.87-6.84 (m, 1H), 3.97 (s, 3H), 2.99-2.93 (m, 1H), 2.89-2.82 (m, 1H), 2.47 (s, 3H), 1.99-1.87 (m, 2H).

### Synthesis of tert-butyl (5-methoxy-2-((1S,2S)-2-(4-methylpyrimidin-2-yl)cyclopropyl)quinolin-7-yl)carbamate

7-bromo-5-methoxy-2-((1S,2S)-2-(4-methylpyrimidin-2-yl)cyclopropyl)quinoline (80 mg, 0.216 mmol), tert-butyl carbamate (38 mg, 0.324 mmol), Pd(OAc)₂ (2.4 mg, 0.011 mmol), Xantphos (13 mg, 0.022 mmol) and Cs₂CO₃ (211 mg, 0.648 mmol) were suspended in 1,4-dioxane (5.0 mL) and degassed for 5 min. The reaction was then heated to 100° C. and stirred for 3 h. The reaction was cooled to room temperature, filtered through Celite® and concentrated in vacuo. The residue was purified by silica gel column chromatography, eluting with 0-100% EtOAc in cyclohexane to give the title compound (75 mg, 85%).

ESI-MS (M+H)+: 407.4, ¹H NMR (400 MHz, CDCl₃) δ 8.44 (d, J=5.1 Hz, 1H), 8.28 (d, J=8.6 Hz, 1H), 7.38-7.35 (m, 1H), 7.17 (s, 1H), 7.13 (d, J=8.6 Hz, 1H), 6.93 (d, J=5.1 Hz, 1H), 6.69 (s, 1H), 3.99 (s, 3H), 2.97-2.91 (m, 1H), 2.86-2.80 (m, 1H), 2.47 (s, 3H), 1.96-1.84 (m, 2H), 1.57 (s, 9H).

### Synthesis of 5-methoxy-2-((1S,2S)-2-(4-methylpyrimidin-2-yl)cyclopropyl)quinolin-7-amine

TFA (0.14 mL, 1.85 mmol) was added to stirred solution of tert-butyl-(5-methoxy-2-((1S,2S)-2-(4-methylpyrimidin-2-yl)cyclopropyl)quinolin-7-yl)carbamate (75 mg, 0.185 mmol) in CHCl₃ (3 mL). The reaction was stirred at room temperature for 3 h then concentrated in vacuo and loaded onto a SCX cartridge. This was washed with 3:1 DCM:MeOH and eluted with 3:1 DCM: 7 N NH₃ in MeOH then concentrated in vacuo to give the title compound (52 mg, 92%).

¹H NMR (400 MHz, DMSO) δ 8.57 (d, J=5.1 Hz, 1H), 8.10 (d, J=8.3 Hz, 1H), 7.23 (d, J=5.6 Hz, 1H), 7.07 (d, J=8.3 Hz, 1H), 6.50 (d, J=1.3 Hz, 1H), 6.43 (d, J=1.8 Hz, 1H), 5.72 (s, 2H), 4.14 (q, J=5.2 Hz, 1H), 3.92 (s, 3H), 2.79-2.67 (m, 2H), 2.47 (s, 3H), 1.83-1.74 (m, 2H).

### Synthesis of 1-(6-cyclopropyl-2-(((5-methoxy-2-((1S,2S)-2-(4-methylpyrimidin-2-yl)cyclopropyl) quinolin-7-yl)amino)methyl)imidazo[1,2-a]pyridin-8-yl)-3-methylimidazolidine-2,4-dione

Ti(iPrO)₄ (0.25 mL, 0.849 mmol) was added to a stirred solution of 5-methoxy-2-((1S,2S)-2-(4-methylpyrimidin-2-yl)cyclopropyl)quinolin-7-amine (52 mg, 0.170 mmol) and 6-cyclopropyl-8-(3-methyl-2,4-dioxoimidazolidin-1-yl)imidazo[1,2-a]pyridine-2-carbaldehyde (51 mg, 0.170 mmol) in a 1:1 mixture of MeOH (4.0 mL) and DCM (4.0 mL). The reaction was then heated at 50° C. in a sealed tube for 16 h. The reaction was cooled to room temperature, NaCNBH₃ (37 mg, 0.594 mmol) was added then the reaction was then stirred at room temperature for 2 h. The reaction was quenched with water (1.0 mL). The reaction mixture was then filtered through Celite® then concentrated in vacuo. The residue was purified by silica gel column chromatography, eluting with 0-10% (7N NH₃ in MeOH) in DCM then by preparative HPLC to give the title compound (50 mg, 46%).

ESI-MS (M+H)+: 598.5, ¹H NMR (400 MHz, DMSO) δ 8.56 (d, J=5.1 Hz, 1H), 8.32 (d, J=1.0 Hz, 1H), 8.11 (d, J=8.8 Hz, 1H), 7.83 (s, 1H), 7.40 (d, J=1.5 Hz, 1H), 7.22 (d, J=5.1 Hz, 1H), 7.11 (d, J=8.6 Hz, 1H), 6.78 (dd, J=5.8, 5.8 Hz, 1H), 6.60 (d, J=1.8 Hz, 1H), 6.45 (d, J=1.5 Hz, 1H), 5.01 (s, 2H), 4.54 (d, J=5.6 Hz, 2H), 3.94 (s, 3H), 3.03 (s, 3H), 2.77-2.66 (m, 2H), 2.46 (s, 3H), 2.03-1.95 (m, 1H), 1.81-1.72 (m, 2H), 0.99 (ddd, J=4.3, 6.3, 8.3 Hz, 2H), 0.73-0.68 (m, 2H).

### Example 29

### Synthesis of rac-1-(6-cyclopropyl-2-(((4-(dimethylamino)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (I-29)

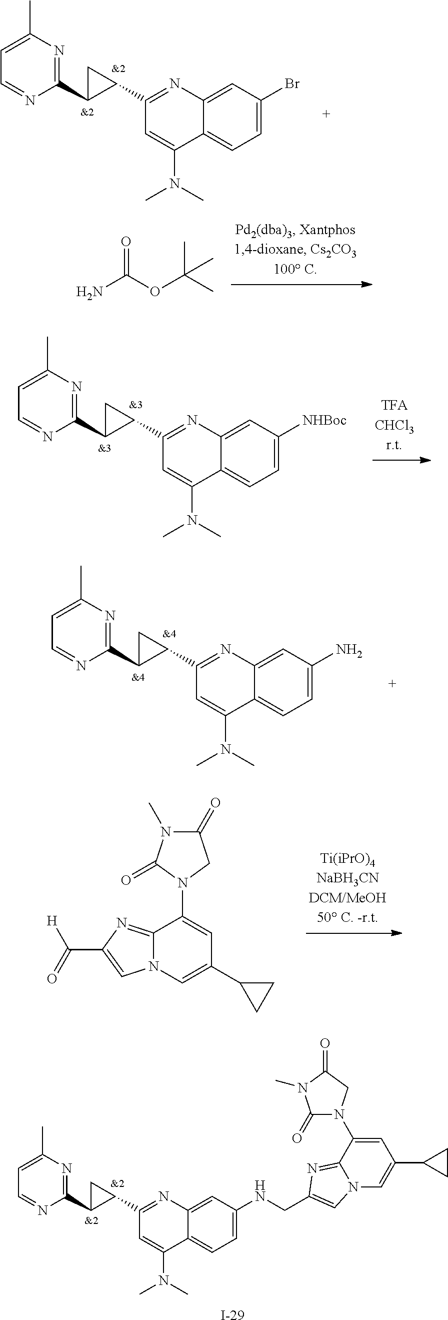

### Synthesis of rac-tert-butyl (4-(dimethylamino)-2-((1S*,2S*)-2-(4-methylpyrimidin-2-yl)cyclopropyl)quinolin-7-yl)carbamate

A mixture of rac-7-bromo-N,N-dimethyl-2-((1S*,2S*)-2-(4-methylpyrimidin-2-yl)cyclopropyl)quinolin-4-amine (150 mg, 0.39 mmol), tert-butyl carbamate (69 mg, 0.59 mmol), Pd₂(dba)₃ (36 mg, 0.039 mmol), Xantphos (45 mg, 0.078 mmol) and Cs₂CO₃ (260 mg, 0.78 mmol) in 1,4-dioxane (4.0 mL) was degassed with N₂ and heated at 100° C. for 18 h. The mixture was filtered through Celite® then concentrated in vacuo and purified by silica gel column chromatography, eluting with 0-100% EtOAc in cyclohexane to give the title compound (120 mg, 70%) as a yellow gum.

ESI-MS (M+H)+: 420.4, ¹H NMR (400 MHz, DMSO) δ 9.65 (s, 1H), 8.54 (d, J=5.1 Hz, 1H), 7.99-7.96 (m, 1H), 7.88 (d, J=9.2 Hz, 1H), 7.47 (dd, J=2.3, 9.2 Hz, 1H), 7.20-7.18 (m, 1H), 6.81 (s, 1H), 2.95 (s, 6H), 2.78-2.65 (m, 2H), 2.43 (s, 3H), 1.86-1.80 (m, 1H), 1.76-1.70 (m, 1H), 1.53 (s, 9H).

### Synthesis of rac-N⁴,N⁴-dimethyl-2-((1S*,2S*)-2-(4-methylpyrimidin-2-yl)cyclopropyl)quinoline-4,7-diamine

A solution of rac-tert-butyl (4-(dimethylamino)-2-((1S*,2S*)-2-(4-methylpyrimidin-2-yl)cyclopropyl)quinolin-7-yl)carbamate (115 mg, 0.274 mmol) in TFA (2.5 mL) and CHCl₃ (2.7 mL) was stirred at room temperature for 2 h then concentrated in vacuo. The residue was dissolved in NaHCO₃ (sat. aq., 50 mL) and extracted with DCM (3×50 mL). The combined organic layers were dried (MgSO₄), filtered and concentrated in vacuo to give the title compound (79 mg, 90%) as a yellow gum.

ESI-MS (M+H)+: 320.2, ¹H NMR (400 MHz, DMSO) δ 8.53 (d, J=5.0 Hz, 1H), 7.71-7.66 (m, 1H), 7.20-7.17 (m, 1H), 6.81-6.79 (m, 2H), 6.58 (s, 1H), 5.58 (s, 2H), 2.91 (br s, 6H), 2.75-2.69 (m, 1H), 2.64-2.57 (m, 1H), 2.43 (s, 3H), 1.82-1.75 (m, 1H), 1.73-1.65 (m, 1H).

### Synthesis of rac-1-(6-cyclopropyl-2-(((4-(dimethylamino)-2-((1S*,2S*)-2-(4-methylpyrimidin-2-yl) cyclopropyl)quinolin-7-yl)amino)methyl)imidazo[1,2-a]pyridin-8-yl)-3-methylimidazolidine-2,4-dione

Ti(iPrO)₄ (0.26 mL, 0.88 mmol) was added to a stirred solution of rac-N,N-dimethyl-2-((1S*,2S*)-2-(4-methylpyrimidin-2-yl)cyclopropyl)quinoline-4,7-diamine (49 mg, 0.18 mmol) and 6-cyclopropyl-8-(3-methyl-2,4-dioxoimidazolidin-1-yl)imidazo[1,2-a]pyridine-2-carbaldehyde (53 mg, 0.178 mmol) in DCM (1.8 mL) and MeOH (1.8 mL). The reaction was sealed and stirred at 50° C. for 18 h. The mixture was cooled to room temperature and further Ti(iPrO)₄ (0.26 mL, 0.88 mmol) was added. The mixture was stirred at 50° C. for 2 h then cooled to room temperature. NaCNBH₃ (39 mg, 0.62 mmol) was added and the mixture was stirred at room temperature for 18 h. Water (1.0 mL) was added and the mixture concentrated in vacuo onto silica gel then purified by silica gel column chromatography, eluting with 1-20% (7N NH₃ in MeOH) in DCM then by preparative HPLC to give the title compound (12 mg, 12%) as a yellow solid (as a formic acid salt).

ESI-MS (M+H)+: 560.3, ¹H NMR (400 MHz, DMSO) δ 8.85-8.84 (m, 1H), 8.54 (d, J=5.1 Hz, 1H), 8.45 (s, 0.5H), 8.25-8.24 (m, 1H), 8.12-8.08 (m, 1H), 7.74 (s, 1H), 7.35 (d, J=1.6 Hz, 1H), 7.28 (t, J=6.1 Hz, 1H), 7.23-7.19 (m, 2H), 6.63 (s, 1H), 4.95 (s, 2H), 4.62 (d, J=6.0 Hz, 2H), 2.99 (s, 3H), 2.81-2.70 (m, 2H), 2.43 (s, 3H), 1.97-1.90 (m, 1H), 1.84-1.75 (m, 2H), 0.97-0.91 (m, 2H), 0.67-0.62 (m, 2H).

The compounds in Table 1 were synthesised in a similar manner to rac-1-(6-cyclopropyl-2-(((4-(dimethylamino)-2-((1S*,2S*)-2-(4-methylpyrimidinin-2-yl)cyclopropyl)quinolin-7-yl)amino), methyl)imidazo[1,2-a]pyridin-8-yl)-3-methylimidazolidine-2,4-dione from 6-cyclopropyl-8-(3-methyl-2,4-dioxoimidazolidin-1-yl)imidazo[1,2-a]pyridine-2-carbaldehyde and an appropriate coupling partner.

TABLE 1

| Ex | Compound | Coupling partner | Analytical data |
|---|---|---|---|
| 30 | 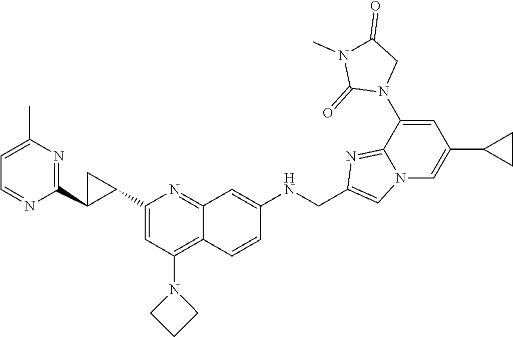 [SMILES: Cc1ccnc([C@H]2C[C@@H]2c2cc(N3CCC3)c3ccc(NCc4cn5cc(C6CC6)cc(N6CC(=O)N(C)C6=O)c5n4)cc3n2)n1] | 4-(azetidin-1-yl)-7- bromo-2-((1S,2S)-2- (4-methylpyrimidin- 2-yl)cyclopropyl) quinoline | ESI-MS (M + H)+: 614.5, ¹H NMR (400 MHz, DMSO) δ 8.50 (d, J = 5.1 Hz, 1H), 8.26-8.24 (m, 2H), 7.73 (s, 1H), 7.64 (d, J = 9.2 Hz, 1H), 7.34 (d, J = 1.5 Hz, 1H), 7.17 (d, J = 5.0 Hz, 1H), 6.85 (dd, J = 2.3, 9.2 Hz, 1H), 6.79-6.79 (m, 1H), 6.66 (d, J = 2.3 Hz, 1H), 5.95 (s, 1H), 4.93 (s, 2H), 4.44 (d, J = 5.5 Hz, 2H), 4.34-4.27 (m, 4H), 2.97 (s, 3H), 2.70-2.64 (m, 1H), 2.56-2.52 (m, 1H), 2.42- 2.33 (m, 5H), 1.97-1.89 (m, 1H), 1.76-1.70 (m, 1H), 1.66-1.60 (m, 1H), 0.96-0.90 (m, 2H), 0.67- 0.61 (m, 2H). |
| | I-30 | | |
| | 1-(2-(((4-(azetidin-1-yl)-2- | | |
| | ((1S,2S)-2-(4-methylpyrimidin-2- | | |
| | yl)cyclopropyl)quinolin-7- | | |
| | yl)amino)methyl)-6- | | |
| | cyclopropylimidazo[1,2- | | |
| | a]pyridin-8-yl)-3- | | |
| | methylimidazolidine-2,4-dione, | | |
| | formic acid salt (I-30) | | |
| 31 | 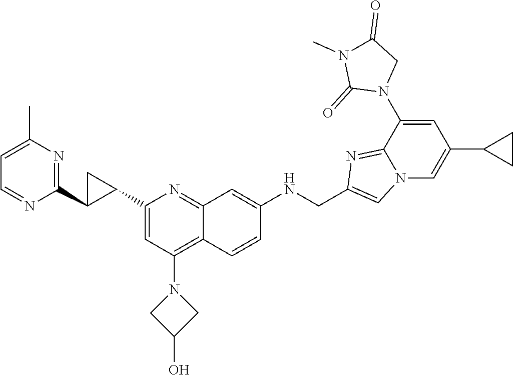 [SMILES: Cc1ccnc([C@H]2C[C@@H]2c2cc(N3CC(O)C3)c3ccc(NCc4cn5cc(C6CC6)cc(N6CC(=O)N(C)C6=O)c5n4)cc3n2)n1] | 1-(7-bromo-2- ((1S,2S)-2-(4- methylpyrimidin-2- yl)cyclopropyl) quinolin- 4-yl)azetidin-3-ol | ESI-MS (M + H)+: 630.5. ¹H NMR (400 MHz, DMSO) δ 8.50 (d, J = 5.1 Hz, 1H), 8.27 (s, 1H), 8.25 (s, 1H), 7.74 (s, 1H), 7.64 (d, J = 9.1 Hz, 1H), 7.34 (s, 1H), 7.16 (d, J = 5.1 Hz, 1H), 6.86 (dd, J = 2.1, 9.2 Hz, 1H), 6.80 (s, 1H), 6.68 (d, J = 1.8 Hz, 1H), 5.98 (s, 1H), 4.92 (s, 2H), 4.62-4.50 (m, 3H), 4.44 (d, J = 5.1 Hz, 2H), 4.03- 3.98 (m, 2H), 2.97 (s, 3H), 2.71- 2.65 (m, 1H), 2.57-2.53 (m, 1H), 2.40 (s, 3H), 1.96-1.88 (m, 1H), 1.78-1.61 (m, 2H), 0.96- 0.89 (m, 2H), 0.67-0.60 (m, 2H). |
| | I-31 | | |
| | 1-(6-cyclopropyl-2-(((4-(3- | | |
| | hydroxyazetidin-1-yl)-2-((1S,2S)- | | |
| | 2-(4-methylpyrimidin-2- | | |
| | yl)cyclopropyl)quinolin-7- | | |
| | yl)amino)methyl)imidazo[1,2- | | |
| | a]pyridin-8-yl)-3- | | |
| | methylimidazolidine-2,4-dione | | |
| | (I-31) | | |
| 32 | 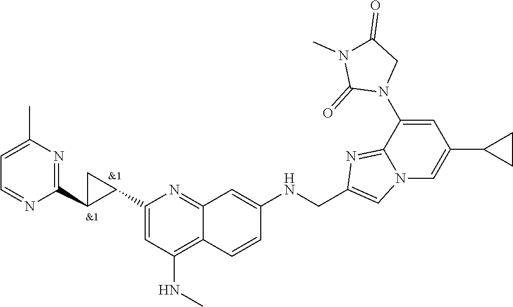 [SMILES: CNc1cc([C@H]2C[C@@H]2c2nccc(C)n2)nc2cc(NCc3cn4cc(C5CC5)cc(N5CC(=O)N(C)C5=O)c4n3)ccc12] | rac-7-bromo-N- methyl-2- ((1S*,2S*)- 2-(4-methyl- pyrimidin-2-yl) cyclopropyl) quinolin- 4-amine | ESI-MS (M + H)+: 588.4, ¹H NMR (400 MHz, DMSO) δ 8.52 (d, J = 5.0 Hz, 1H), 8.27-8.26 (m, 1H), 7.81 (d, J = 9.2 Hz, 1H), 7.75 (s, 1H), 7.36 (d, J = 1.5 Hz, 1H), 7.18 (d, J = 5.1 Hz, 2H), 6.88 (dd, J = 2.3, 9.0 Hz, 1H), 6.66 (d, J = 2.3 Hz, 2H), 6.17 (s, 1H), 4.95 (s, 2H), 4.46 (d, J = 5.6 Hz, 2H), 2.99 (s, 3H), 2.88 (d, J = 4.6 Hz, 3H), 2.75-2.68 (m, 1H), 2.59-2.54 (m, 1H), 2.42 (s, 3H), 1.98-1.91 (m, 1H), 1.80-1.75 (m, 1H), 1.68- 1.63 (m, 1H), 0.97-0.92 (m, 2H), 0.68-0.63 (m, 2H). |
| | I-32 | | |
| | rac-1-(6-cyclopropyl-2-(((4- | | |
| | (methylamino)-2-((1S*,2S*)-2- | | |
| | (4-methylpyrimidin-2- | | |
| | yl)cyclopropyl)quinolin-7- | | |
| | yl)amino)methyl)imidazo[1,2- | | |
| | a]pyridin-8-yl)-3- | | |
| | methylimidazolidine-2,4-dione, | | |
| | formic acid salt (I-32) | | |
| 33 | 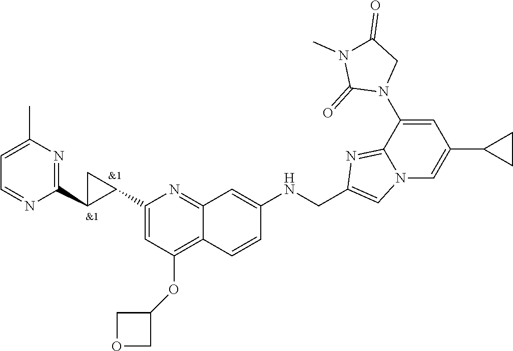 [SMILES: Cc1ccnc([C@H]2C[C@@H]2c2cc(OC3COC3)c3ccc(NCc4cn5cc(C6CC6)cc(N6CC(=O)N(C)C6=O)c5n4)cc3n2)n1] | rac-7-bromo-2- ((1S*,2S*)-2-(4- methylpyrimidin-2- yl)cyclopropyl)-4- (oxetan-3- yloxy)quinoline | ESI-MS (M + H)+: 630, Purity: ¹H NMR (400 MHz, DMSO) δ 8.52 (d, J = 5.0 Hz, 1H), 8.27 (d, J = 1.1 Hz, 1H), 7.85 (d, J = 9.0 Hz, 1H), 7.77 (s, 1H), 7.37 (d, J = 1.5 Hz, 1H), 7.18 (d, J = 5.1 Hz, 1H), 7.03 (dd, J = 2.3, 9.0 Hz, 1H), 6.81 (dd, J = 5.8, 5.8 Hz, 1H), 6.74 (d, J = 2.3 Hz, 1H), 6.39 (s, 1H), 5.51- 5.44 (m, 1H), 5.07-4.99 (m, 2H), 4.96 (s, 2H), 4.67-4.62 (m, 2H), 4.49 (d, J = 5.6 Hz, 2H), 2.99 (s, 3H), 2.76-2.61 (m, 2H), 2.42 (s, 3H), 1.98-1.90 (m, 1H), 1.78-1.66 (m, 2H), 0.97-0.91 (m, 2H), 0.68- 0.63 (m, 2H). |
| | I-33 | | |
| | rac-1-(6-cyclopropyl-2-(((2- | | |
| | ((1S*,2S*)-2-(4-methylpyrimidin- | | |
| | 2-yl)cyclopropyl)-4-(oxetan-3- | | |
| | yloxy)quinolin-7-yl)amino) | | |
| | methyl)imidazo[1,2-a]pyridin-8- | | |
| | yl)-3-methylimidazolidine-2,4- | | |
| | dione (I-33) | | |
| 34 | 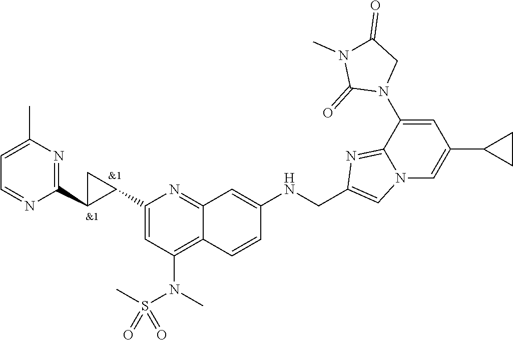 [SMILES: Cc1ccnc([C@H]2C[C@@H]2c2cc(N(C)S(C)(=O)=O)c3ccc(NCc4cn5cc(C6CC6)cc(N6CC(=O)N(C)C6=O)c5n4)cc3n2)n1] | rac-N-(7-bromo-2- ((1S*,2S*)-2-(4- methylpyrimidin-2- yl)cyclopropyl) quinolin- 4-yl)-N-methyl- methanesulfonamide | ESI-MS (M + H)+: 666.5. ¹H NMR (400 MHz, DMSO) δ 8.53 (d, J = 5.1 Hz, 1H), 8.27 (d, J = 1.1 Hz, 1H), 7.81-7.76 (m, 2H), 7.37-7.35 (m, 2H), 7.19 (d, J = 5.4 Hz, 1H), 7.13 (dd, J = 2.4, 9.2 Hz, 1H), 6.97 (dd, J = 6.0, 6.0 Hz, 1H), 6.81 (d, J = 2.3 Hz, 1H), 4.96 (s, 2H), 4.52 (d, J = 5.6 Hz, 2H), 3.26 (s, 3H), 3.18 (s, 3H), 2.99 (s, 3H), 2.75 (dd, J = 7.1, 7.1 Hz, 2H), 2.42 (s, 3H), 1.97-1.90 (m, 1H), 1.83-1.72 (m, 2H), 0.97- 0.91 (m, 2H), 0.68-0.63 (m, 2H). |
| | I-34 | | |
| | rac-N-(7-(((6-cyclopropyl-8-(3- | | |
| | methyl-2,4-dioxoimidazolidin-1- | | |
| | yl)imidazo[1,2-a]pyridin-2- | | |
| | yl)methyl)amino)-2-((1S*,2S*)-2- | | |
| | (4-methylpyrimidin-2- | | |
| | yl)cyclopropyl)quinolin-4-yl)-N- | | |
| | methylmethanesulfonamide, | | |
| | formic acid salt (I-34) | | |
| 35 | 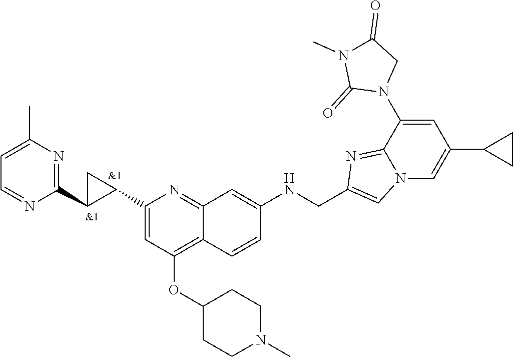 [SMILES: Cc1ccnc([C@H]2C[C@@H]2c2cc(OC3CCN(C)CC3)c3ccc(NCc4cn5cc(C6CC6)cc(N6CC(=O)N(C)C6=O)c5n4)cc3n2)n1] | rac-7-bromo-4-((1- methylpiperidin-4- yl)oxy)-2- ((1S*,2S*)- 2-(4-methyl pyrimidin-2-yl) cyclopropyl) quinoline | ESI-MS (M + H)+: 672, Purity: ¹H NMR (400 MHz, DMSO) δ 8.52 (d, J = 5.1 Hz, 1H), 8.28-8.26 (m, 1H), 7.80-7.74 (m, 2H), 7.36 (d, J = 1.8 Hz, 1H), 7.18 (d, J = 5.3 Hz, 1H), 6.98 (dd, J = 2.3, 9.1 Hz, 1H), 6.82 (s, 1H), 6.77-6.70 (m, 2H), 4.96 (s, 2H), 4.74-4.66 (m, 1H), 4.48 (d, J = 5.6 Hz, 2H), 2.99 (s, 3H), 2.74-2.58 (m, 4H), 2.42 (s, 3H), 2.35-2.25 (m, 2H), 2.20 (s, 3H), 2.03-1.90 (m, 3H), 1.79-1.64 (m, 4H), 0.94 (ddd, J = 4.4, 6.4, 8.3 Hz, 2H), 0.68- 0.63 (m, 2H). |
| | I-35 | | |
| | rac-1-(6-cyclopropyl-2-(((4-((1- | | |
| | methylpiperidin-4-yl)oxy)-2- | | |
| | ((1S*,2S*)-2-(4-methylpyrimidin- | | |
| | 2-yl)cyclopropyl)quinolin-7- | | |
| | yl)amino)methyl)imidazo[1,2- | | |
| | a]pyridin-8-yl)-3-methyl- | | |
| | imidazolidine-2,4-dione (I-35) | | |
| 36 | 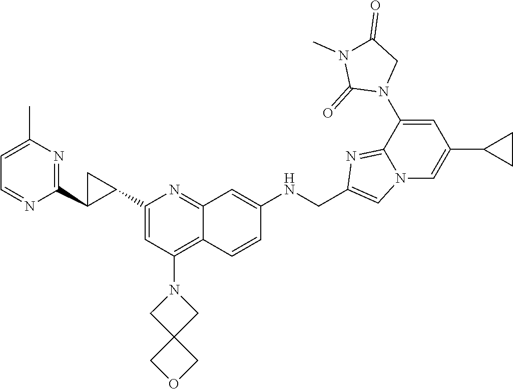 [SMILES: Cc1ccnc([C@H]2C[C@@H]2c2cc(N3CC4(COC4)C3)c3ccc(NCc4cn5cc(C6CC6)cc(N6CC(=O)N(C)C6=O)c5n4)cc3n2)n1] | 6-(7-bromo-2- ((1S,2S)-2-(4- methylpyrimidin-2- yl)cyclopropyl) quinolin-4- yl)-2-oxa- 6-azaspiro[3.3] heptane | ESI-MS (M + H)+: 656.4, ¹H NMR (400 MHz, DMSO) δ 8.55 (d, J = 5.1 Hz, 1H), 8.31 (s, 2H), 7.79 (s, 1H), 7.67-7.64 (m, 1H), 7.40 (d, J = 1.5 Hz, 1H), 7.21 (d, J = 5.1 Hz, 1H), 6.91 (dd, J = 2.4, 9.2 Hz, 1H), 6.73-6.69 (m, 3H), 6.08 (s, 1H), 4.99 (s, 2H), 4.80- 4.78 (m, 4H), 4.52-4.46 (m, 6H), 3.03 (s, 3H), 2.74-2.67 (m, 1H), 2.45 (s, 3H), 2.02-1.95 (m, 1H), 1.79-1.74 (m, 1H), 1.70-1.64 (m, 1H), 1.01-0.95 (m, 2H), 0.72- 0.67 (m, 2H). Cyclopropyl 1H signal obscured by DMSO signal. |
| | I-36 | | |
| | 1-(6-cyclopropyl-2-(((2-((1S,2S)- | | |
| | 2-(4-methylpyrimidin-2- | | |
| | yl)cyclopropyl)-4-(2-oxa-6- | | |
| | azaspiro[3.3]heptan-6- | | |
| | yl)quinolin-7-yl)amino) | | |
| | methyl)imidazo[1,2-a]pyridin-8- | | |
| | yl)-3-methylimidazolidine-2,4- | | |
| | dione, formic acid salt (I-36) | | |
| 37 | 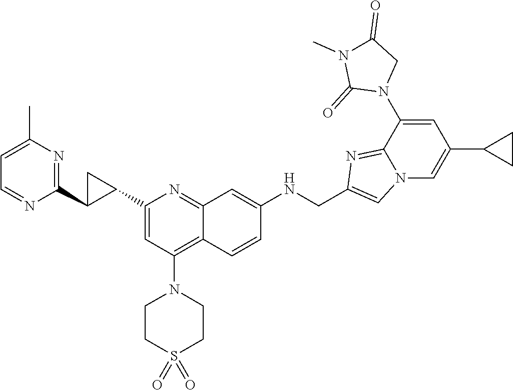 [SMILES: Cc1ccnc([C@H]2C[C@@H]2c2cc(N3CCS(=O)(=O)CC3)c3ccc(NCc4cn5cc(C6CC6)cc(N6CC(=O)N(C)C6=O)c5n4)cc3n2)n1] | 4-(7-bromo-2- ((1S,2S)-2-(4- methylpyrimidin- 2-yl) cyclopropyl) quinolin- 4-yl)thiomorpholine 1,1-dioxide | ESI-MS (M + H)+: 692.5, ¹H NMR (400 MHz, DMSO) δ 8.51 (d, J = 5.0 Hz, 1H), 8.31 (s, 0.8H), 8.26-8.24 (m, 1H), 7.75-7.71 (m, 2H), 7.34 (d, J = 1.5 Hz, 1H), 7.16 (d, J = 5.1 Hz, 1H), 7.04- 6.99 (m, 1H), 6.83 (s, 1H), 6.77- 6.75 (m, 2H), 4.94 (s, 2H), 4.49- 4.45 (m, 2H), 3.53-3.52 (m, 4H), 3.39-3.39 (m, 4H), 2.97 (s, 3H), 2.73-2.61 (m, 2H), 2.40 (s, 3H), 1.97-1.89 (m, 1H), 1.78-1.65 (m, 2H), 0.96-0.90 (m, 2H), 0.66- 0.61 (m, 2H). |
| | I-37 | | |
| | 1-(6-cyclopropyl-2-(((4-(1,1- | | |
| | dioxidothiomorpholino)-2- | | |
| | ((1S,2S)-2-(4-methylpyrimidin-2- | | |
| | yl)cyclopropyl)quinolin-7-yl) | | |
| | amino)methyl)imidazo[1,2- | | |
| | a]pyridin-8-yl)-3- | | |
| | methylimidazolidine-2,4-dione, formic | | |
| | acid salt (I-37) | | |
| 38 | 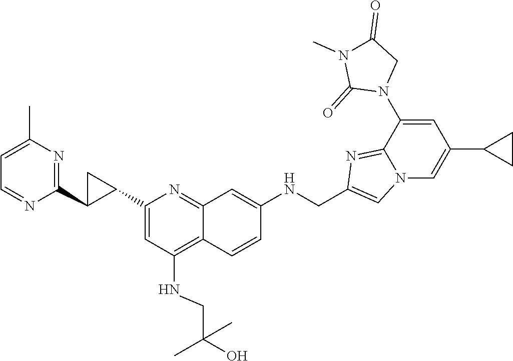 [SMILES: Cc1ccnc([C@H]2C[C@@H]2c2cc(NCC(C)(C)O)c3ccc(NCc4cn5cc(C6CC6)cc(N6CC(=O)N(C)C6=O)c5n4)cc3n2)n1] | 1-((7-bromo-2- ((1S,2S)-2-(4- methylpyrimidin-2- yl)cyclopropyl) quinolin- 4-yl)amino)-2- methylpropan-2-ol | ESI-MS (M + H)+: 646.5, ¹H NMR (400 MHz, DMSO) δ 8.51 (d, J = 5.1 Hz, 1H), 8.28 (s, 1H), 8.26-8.25 (m, 1H), 7.89 (d, J = 9.2 Hz, 1H), 7.73 (s, 1H), 7.34- 7.34 (m, 1H), 7.17 (d, J = 4.9 Hz, 1H), 6.91-6.87 (m, 1H), 6.71- 6.63 (m, 3H), 6.33 (s, 1H), 4.93 (s, 2H), 4.48-4.43 (m, 2H), 3.24- 3.21 (m, 2H), 2.97 (s, 3H), 2.71- 2.67 (m, 1H), 2.40 (s, 3H), 1.95- 1.89 (m, 1H), 1.76-1.72 (m, 1H), 1.66-1.62 (m, 1H), 1.17 (s, 6H), 0.95-0.89 (m, 2H), 0.66- 0.61 (m, 2H). Cyclopropyl CH masked by DMSO. |
| | I-38 | | |
| | 1-(6-cyclopropyl-2-(((4-((2- | | |
| | hydroxy-2-methylpropyl)amino)- | | |
| | 2-((1S,2S)-2-(4-methylpyrimidin- | | |
| | 2-yl)cyclopropyl)quinolin-7- | | |
| | yl)amino)methyl)imidazo[1,2- | | |
| | a]pyridin-8-yl)-3- | | |
| | methylimidazolidine-2,4-dione | | |
| | (I-38) | | |
| 39 | 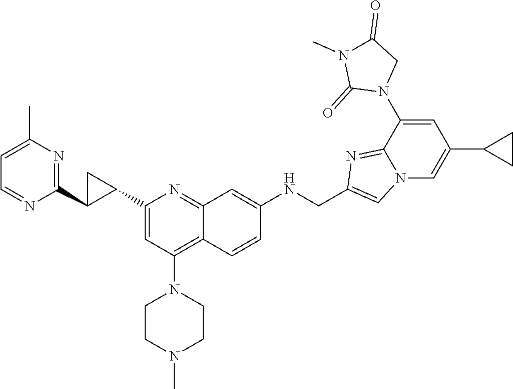 [SMILES: Cc1ccnc([C@H]2C[C@@H]2c2cc(N3CCN(C)CC3)c3ccc(NCc4cn5cc(C6CC6)cc(N6CC(=O)N(C)C6=O)c5n4)cc3n2)n1] | 7-bromo-4-(4- methylpiperazin-1- yl)-2-((1S,2S)-2-(4- methylpyrimidin-2- yl)cyclopropyl) quinoline | ESI-MS (M + H)+: 657, ¹H NMR (400 MHz, DMSO) δ 8.50 (d, J = 5.1 Hz, 1H), 8.25 (d, J = 1.1 Hz, 1H), 8.14 (s, 1H), 7.74 (s, 1H), 7.63 (d, J = 9.0 Hz, 1H), 7.34 (d, J = 1.5 Hz, 1H), 7.16 (d, J = 5.1 Hz, 1H), 6.98 (dd, J = 2.4, 9.2 Hz, 1H), 6.73- 6.72 (m, 2H), 6.67 (s, 1H), 4.94 (s, 2H), 4.46 (d, J = 5.5 Hz, 2H), 3.12-3.09 (m, 5H), 2.97 (s, 3H), 2.73-2.67 (m, 1H), 2.64- 2.56 (m, 4H), 2.40 (s, 3H), 2.29 (s, 3H), 1.96-1.88 (m, 1H), 1.77- 1.72 (m, 1H), 1.69-1.64 (m, 1H), 0.95-0.89 (m, 2H), 0.66- 0.61 (m, 2H). |
| | I-39 | | |
| | 1-(6-cyclopropyl-2-(((4-(4- | | |
| | methylpiperazin-1-yl)-2-((1S,2S)- | | |
| | 2-(4-methylpyrimidin-2-yl) | | |
| | cyclopropyl)quinolin-7-yl)amino) | | |
| | methyl)imidazo[1,2-a]pyridin-8- | | |
| | yl)-3-methylimidazolidine-2,4- | | |
| | dione, formic acid salt (I-39) | | |

### Examples 40-42

### Synthesis of rac-1-(6-cyclopropyl-2-((1S*,2S)-2-(4-methylpyrimidin-2-yl)cyclopropyl)-4-morpholinoquinolin-7-yl)amino)methyl)imidazo[1,2-a]pyridin-8-yl)-3-methylimidazolidine-2,4-dione (I-40)

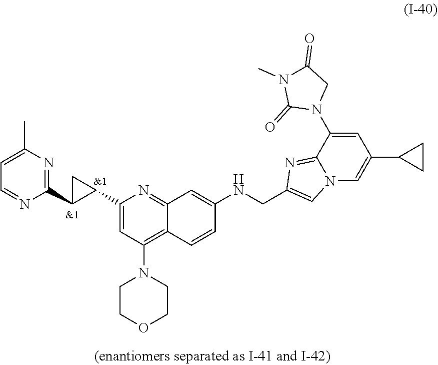 [SMILES: Cc1ccnc([C@H]2C[C@@H]2c2cc(N3CCOCC3)c3ccc(NCc4cn5cc(C6CC6)cc(N6CC(=O)N(C)C6=O)c5n4)cc3n2)n1]

Using a similar procedure to that used for rac-1-(6-cyclopropyl-2-(((4-(dimethylamino)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione, the title compound was prepared from rac-4-(7-bromo-2-((1S*,2S*)-2-(4-methylpyrimidin-2-yl)cyclopropyl)quinolin-4-yl)morpholine (26 mg, 23%) as a mixture of enantiomers.

ESI-MS (M+H)+: 644.3, Purity 99.7%, ¹H NMR (400 MHz, DMSO): δ 8.52 (1H, d, J=5.1 Hz), 8.27 (1H, d, J=1.0 Hz), 7.77 (1H, s), 7.69 (1H, d, J=9.2 Hz), 7.36 (1H, d, J=1.5 Hz), 7.18 (1H, d, J=5.4 Hz), 7.00 (1H, dd, J=2.4, 9.0 Hz), 6.76-6.71 (3H, m), 4.96 (2H, s), 4.48 (2H, d, J=5.6 Hz), 3.86-3.81 (4H, m), 3.10-3.07 (4H, m), 2.99 (3H, s), 2.73-2.68 (1H, m), 2.65-2.60 (1H, m), 2.42 (3H, s), 1.98-1.91 (1H, m), 1.79-1.73 (1H, m), 1.71-1.66 (1H, m), 0.97-0.91 (2H, m), 0.68-0.63 (2H, m).

The enantiomers were separated using SFC (YMC Amylose-C 10×250 mm, 5 um 50/50 IPA (0.1% DEA)/CO2, 15 ml/min, 120 bar, 40 C) to give two enantiomers: 1-(6-cyclopropyl-2-(((2-((1S,2S)-2-(4-methylpyrimidin-2-yl)cyclopropyl)-4-morpholinoquinolin-7-yl)amino)methyl)imidazo[1,2-a]pyridin-8-yl)-3-methylimidazolidine-2,4-dione and 1-(6-cyclopropyl-2-(((2-((1R,2R)-2-(4-methylpyrimidin-2-yl)cyclopropyl)-4-morpholinoquinolin-7-yl)amino)methyl)imidazo[1,2-a]pyridin-8-yl)-3-methylimidazolidine-2,4-dione.

First Eluting Isomer (7.5 mg) (Example 41, I-41)

ESI-MS (M+H)+: 644.6, ¹H NMR (400 MHz, DMSO): d, ppm 8.52 (1H, d, J=5.0 Hz), 8.27 (1H, s), 7.77 (1H, s), 7.70 (1H, d, J=9.0 Hz), 7.36 (1H, d, J=1.6 Hz), 7.18 (1H, d, J=5.1 Hz), 7.00 (1H, dd, J=2.3, 9.0 Hz), 6.76-6.71 (3H, m), 4.96 (2H, s), 4.48 (2H, d, J=5.5 Hz), 3.86-3.82 (4H, m), 3.11-3.07 (4H, m), 2.99 (3H, s), 2.73-2.68 (1H, m), 2.65-2.60 (1H, m), 2.42 (3H, s), 1.98-1.91 (1H, m), 1.79-1.66 (2H, m), 0.97-0.92 (2H, m), 0.68-0.63 (2H, m), RT=6.40 min, 100% e.e.

Second Eluting Isomer (7.3 mg) (Example 42, I-42)

ESI-MS (M+H)+: 644.5, ¹H NMR (400 MHz, DMSO): d, ppm 8.52 (1H, d, J=5.1 Hz), 8.27 (1H, d, J=1.1 Hz), 7.77 (1H, s), 7.69 (1H, d, J=9.0 Hz), 7.36 (1H, d, J=1.5 Hz), 7.18 (1H, d, J=5.1 Hz), 7.00 (1H, dd, J=2.4, 9.0 Hz), 6.77-6.71 (3H, m), 4.96 (2H, s), 4.48 (2H, d, J=5.9 Hz), 3.84 (4H, dd, J=4.4, 4.4 Hz), 3.09 (4H, dd, J=4.1, 4.1 Hz), 2.99 (3H, s), 2.73-2.68 (1H, m), 2.65-2.60 (1H, m), 2.42 (3H, s), 1.98-1.91 (1H, m), 1.79-1.74 (1H, m), 1.71-1.66 (1H, m), 0.97-0.91 (2H, m), 0.68-0.63 (2H, m). RT=8.70 min, 98.9% e.e.

### Example 43

### Synthesis of 1-[6-cyclopropyl-2-[[2-[(1S,2S)-2-(4-methylpyrimidin-2-yl)cyclopropyl]-4-(2-oxa-6-azaspiro[3.3]heptan-6-yl)-7-quinolyl]oxymethyl]imidazo[1,2-a]pyridin-8-yl]-3-methyl-imidazolidine-2,4-dione (I-43)

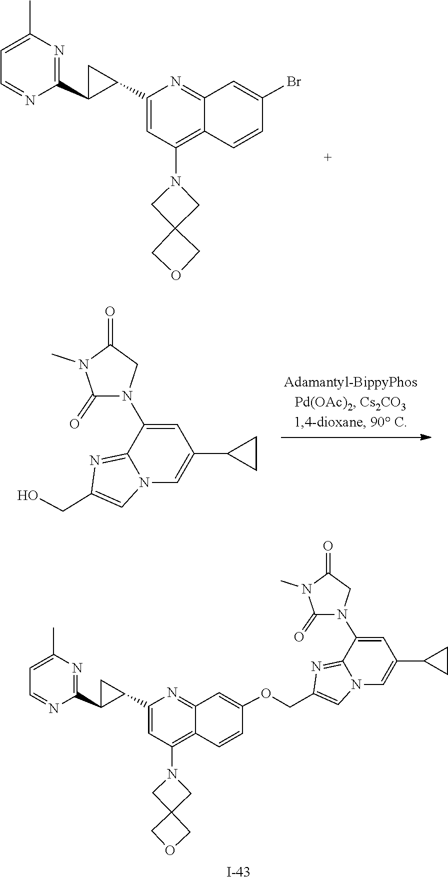

### Synthesis of 1-(6-cyclopropyl-2-(((2-((1S,2S)-2-(4-methylpyrimidin-2-yl)cyclopropyl)-4-(2-oxa-6-aza spiro[3.3]heptan-6-yl)quinolin-7-yl)oxy)methyl)imidazo[1,2-a]pyridin-8-yl)-3-methylimidazolidine-2,4-dione

A suspension of 6-(7-bromo-2-((1S,2S)-2-(4-methylpyrimidin-2-yl)cyclopropyl)quinolin-4-yl)-2-oxa-6-azaspiro[3.3]heptane (110 mg, 0.24 mmol), 1-(6-cyclopropyl-2-(hydroxymethyl) imidazo [1,2-a]pyridin-8-yl)-3-methylimidazolidine-2,4-dione (72 mg, 0.24 mmol), Cs₂CO₃ (240 mg, 0.72 mmol), and Adamantyl-BippyPhos (32 mg, 0.048 mmol) in 1,4-dioxane (1.0 mL) was degassed with N₂ for 10 min. Pd(OAc)₂ (5.4 mg, 0.024 mmol) was added and the reaction mixture was stirred at 90° C. for 18 h. The reaction mixture was filtered through a pad of Celite® and concentrated in vacuo. The residue was purified by silica gel column chromatography, eluting with 0-20% (7N NH₃ in MeOH) in DCM then by preparative HPLC to give the title compound (16 mg, 10%) as a white solid (formic acid salt).

ESI-MS (M+H)+: 657, ¹H NMR (400 MHz, DMSO) δ 8.51 (d, J=5.1 Hz, 1H), 8.30 (d, J=1.0 Hz, 1H), 8.18 (s, 1H), 7.98 (s, 1H), 7.80 (d, J=9.3 Hz, 1H), 7.39 (d, J=1.5 Hz, 1H), 7.28 (d, J=2.5 Hz, 1H), 7.17 (d, J=5.1 Hz, 1H), 7.00 (dd, J=2.7, 9.2 Hz, 1H), 6.24 (s, 1H), 5.30 (s, 2H), 4.91 (s, 2H), 4.74 (s, 4H), 4.46 (s, 4H), 2.97 (s, 3H), 2.75-2.69 (m, 1H), 2.62-2.57 (m, 1H), 2.41 (s, 3H), 2.00-1.90 (m, 1H), 1.80-1.75 (m, 1H), 1.70-1.64 (m, 1H), 0.98-0.92 (m, 2H), 0.69-0.64 (m, 2H).

### Example 44

### Synthesis of 8-((1S,2S)-2-(3-chlorophenyl) cyclopropyl)-7-((6-cyclopropylimidazo[1,2-a]pyridin-2-yl) methyl)-7H-purine (I-44)

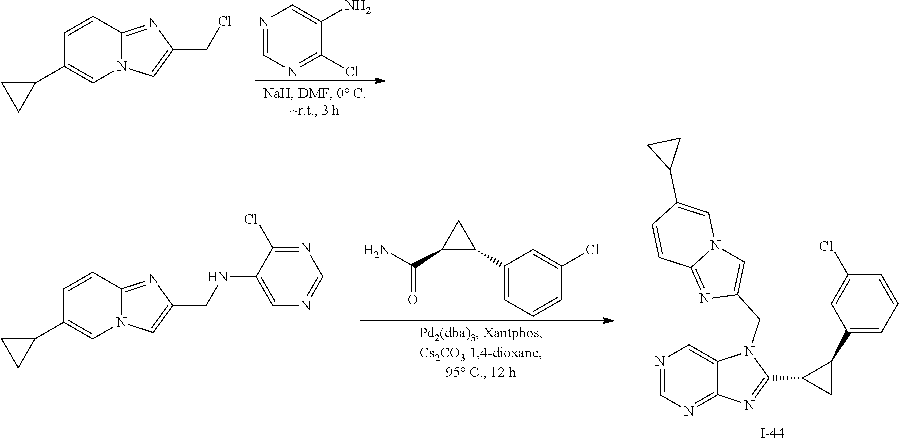

### Synthesis of 4-chloro-N-((6-cyclopropylimidazo[1,2-a]pyridin-2-yl)methyl)pyrimidin-5-amine

To a solution of 4-chloropyrimidin-5-amine (1.2 g, 9.3 mmol) in DMF (40 mL) was added NaH (0.48 g, 12 mmol, 60% dispersion in the mineral oil) at 0° C. After stirring at room temperature for 1 h under N₂, the reaction mixture was added a solution of 2-(chloromethyl)-6-cyclopropylimidazo[1,2-a] pyridine (1.6 g, 7.7 mmol) in DMF (10 mL). The resulting mixture was stirred at room temperature for another 2 h. The reaction mixture was quenched with water (60 mL). The aqueous phase was extracted with EtOAc (60 mL×3). The combined organic layers were washed with brine (100 mL), dried over Na₂SO₄, and then concentrated in vacuo to give the crude, which was purified by column chromatography on silica gel, eluting with 0˜50% EtOAc/PE to give 4-chloro-N-((6-cyclopropylimidazo[1,2-a]pyridin-2-yl)methyl)pyrimidin-5-amine (330 mg, 14%) as a yellow oil. ESI-MS [M+H]+: 300.1

### Synthesis of 8-((1S,2S)-2-(3-chlorophenyl) cyclopropyl)-7-((6-cyclopropylimidazo[1,2-a]pyridin-2-yl) methyl)-7H-purine

To a mixture of 4-chloro-N-((6-cyclopropylimidazo[1,2-a]pyridin-2-yl)methyl)pyrimidin-5-amine (50 mg, 0.17 mmol), (1S,2S)-2-(3-chlorophenyl)cyclopropane-1-carboxamide (40 mg, 0.20 mmol) and Cs₂CO₃ (166 mg, 0.51 mmol) in 1,4-dioxane (5.0 mL) was added Pd₂(dba)₃ (31 mg, 0.034 mmol) and Xantphos (20 mg, 0.034 mmol). The reaction mixture was stirred at 95° C. for 12 h under N₂. The reaction mixture was filtered through Celite® and the filter cake was washed with DCM/MeOH (V/V=10/1, 50 mL). The filtrate was concentrated in vacuo to give the crude, which was purified by preparative TLC, eluting with 6% MeOH/DCM to give 8-((1S,2S)-2-(3-chlorophenyl)cyclopropyl)-7-((6-cyclopropylimidazo[1,2-a]pyridin-2-yl)methyl)-7H-purine (20 mg, 27%) as a yellow solid.

ESI-MS [M+H]+: 441.2, ¹H NMR (400 MHz, DMSO) δ 9.16 (s, 1H), 8.93 (s, 1H), 8.21 (s, 1H), 7.75 (s, 1H), 7.40-7.25 (m, 4H), 7.21-7.12 (m, 1H), 7.06-6.94 (m, 1H), 5.90-5.75 (m, 2H), 3.05-2.92 (m, 1H), 2.75-2.63 (m, 1H), 2.05-1.89 (m, 2H), 1.85-1.72 (m, 1H), 1.04-0.88 (m, 2H), 0.78-0.63 (m, 2H).

### Example 45

### Synthesis of 2-((1S,2S)-2-(3-chlorophenyl)cyclopropyl)-N-((6-cyclopropylimidazo[1,2-a]pyridin-2-yl)methyl)-1H-benzo[d]imidazol-6-amine (I-45)

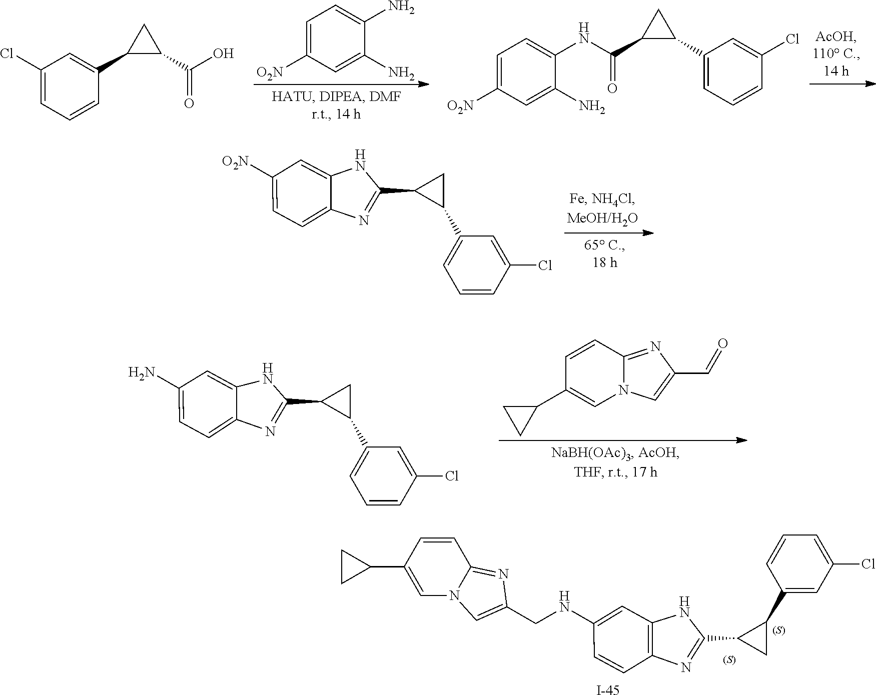

### Synthesis of (1S,2S)—N-(2-amino-4-nitrophenyl)-2-(3-chlorophenyl)cyclopropane-1-carboxamide

A mixture of (1S,2S)-2-(3-chlorophenyl)cyclopropane-1-carboxylic acid (236 mg, 1.2 mmol) 4-nitrobenzene-1,2-diamine (153 mg, 1 mol), HATU (950 mg, 2.5 mmol) and DIPEA (645 mg, 5 mmol) in DMF (5 mL) was stirred at 60° C. under N₂ for 14 h. Water (50 ml) was added and the mixture was extracted with EtOAc (30 mL×3). The combined organic layers were washed with brine (30 mL), dried over Na₂SO₄ and concentrated in vacuo. The residue was purified by column chromatography (eluent: DCM/MeOH=0˜5%) to give (1S,2S)—N-(2-amino-4-nitrophenyl)-2-(3-chlorophenyl)cyclopropane-1-carboxamide (200 mg, 60%) as yellow oil. ESI-MS [M+H]⁺: 332.1.

### Synthesis of 2-((1S,2S)-2-(3-chlorophenyl)cyclopropyl)-6-nitro-1H-benzo[d]imidazole

A solution of (1S, 2S)—N-(2-amino-4-nitrophenyl)-2-(3-chlorophenyl)cyclopropane-1-carboxamide (200 mg, 0.6 mmol) in AcOH (10 mL) was stirred at 110° C. for 14 h. The mixture was evaporated to remove AcOH, residue was diluted with NaHCO₃ (sat. aq., 50 mL) and extracted with EtOAc (30 mL×3). The combined organic layers were washed with brine (30 mL), dried over Na₂SO₄ and concentrated in vacuo. The residue was purified by preparative TLC (eluent: DCM/MeOH=20/1) to give 2-((1S,2S)-2-(3-chlorophenyl)cyclopropyl)-6-nitro-1H-benzo[d]imidazole (180 mg, 95%) as a yellow solid. ESI-MS [M+H]⁺: 314.1.

### Synthesis of 2-((1S,2S)-2-(3-chlorophenyl)cyclopropyl)-1H-benzo[d]imidazol-6-amine

A mixture of 2-((1S,2S)-2-(3-chlorophenyl)cyclopropyl)-6-nitro-1H-benzo[d]imidazole (180 mg, 0.57 mmol), Fe (160 mg, 2.85 mmol) and NH₄Cl (302 mg, 5.7 mmol) in MeOH/H₂O (6 mL/2 mL) was stirred at 65° C. under N₂ for 18 h. The reaction mixture was filtered through Celite® and the filter cake was washed with DCM/MeOH (10/1, 50 mL). To the filtrate was added water (30 mL) and extracted with DCM/MeOH (10/1, 50 mL×3). The combined organic layers were washed with brine (30 mL), dried over Na₂SO₄ and concentrated in vacuo to give 2-((1S,2S)-2-(3-chlorophenyl)cyclopropyl)-1H-benzo[d]imidazol-6-amine (160 mg, crude) as brown solid, which was used in the next step without further purification. ESI-MS [M+H]⁺: 284.1.

### Synthesis of 2-((1S,2S)-2-(3-chlorophenyl)cyclopropyl)-N-((6-cyclopropylimidazo[1,2-a]pyridin-2-yl)methyl)-1H-benzo[d]imidazol-6-amine

To a mixture of 2-((1S,2S)-2-(3-chlorophenyl)cyclopropyl)-1H-benzo[d]imidazol-6-amine (100 mg, 0.35 mmol) and 6-cyclopropylimidazo[1,2-a]pyridine-2-carbaldehyde (65 mg, 0.35 mol) in THF (15 mL) was added acetic acid (0.1 mL). The resulting solution was stirred for 16 h at room temperature. NaBH(OAc)₃ (148 mg, 0.7 mmol) was added and the solution was stirred for additional 1 hour. The reaction was quenched with NaHCO₃ (sat. aq., 50 mL) and extracted with ethyl acetate (30 mL×3). The combined organic layers were washed with brine (30 mL), dried over Na₂SO₄ and concentrated in vacuo to give the crude, which was purified with preparative TLC (eluent: DCM/MeOH: 10/1) to give 2-((1S,2S)-2-(3-chlorophenyl)cyclopropyl)-N-((6-cyclopropylimidazo[1,2-a]pyridin-2-yl)methyl)-1H-benzo[d]imidazol-6-amine (23 mg, 14%) as red solid.

ESI-MS [M+H]⁺: 454.2, ¹H NMR (400 MHz, DMSO) δ 11.71 (s, 1H), 8.28 (s, 1H), 7.61 (s, 1H), 7.37 (d, J=9.3 Hz, 1H), 7.33-7.27 (m, 2H), 7.25-7.22 (m, 1H), 7.18-7.14 (m, 2H), 6.95 (dd, J=9.3, 1.7 Hz, 1H), 6.59-6.42 (m, 2H), 5.86 (s, 1H), 4.33 (d, J=3.8 Hz, 2H), 2.46-2.41 (m, 1H), 2.29-2.24 (m, 1H), 1.93-1.86 (m, 1H), 1.73-1.68 (m, 1H), 1.58-1.53 (m, 1H), 0.92-0.83 (m, 2H), 0.70-0.60 (m, 2H)

### Example 46

### Synthesis of 2-((1S, 2S)-2-(3-chlorophenyl)cyclopropyl)-N-((6-cyclopropylimidazo[1,2-a]pyridin-2-yl)methyl)-1H-benzo[d]imidazol-7-amine (I-46)

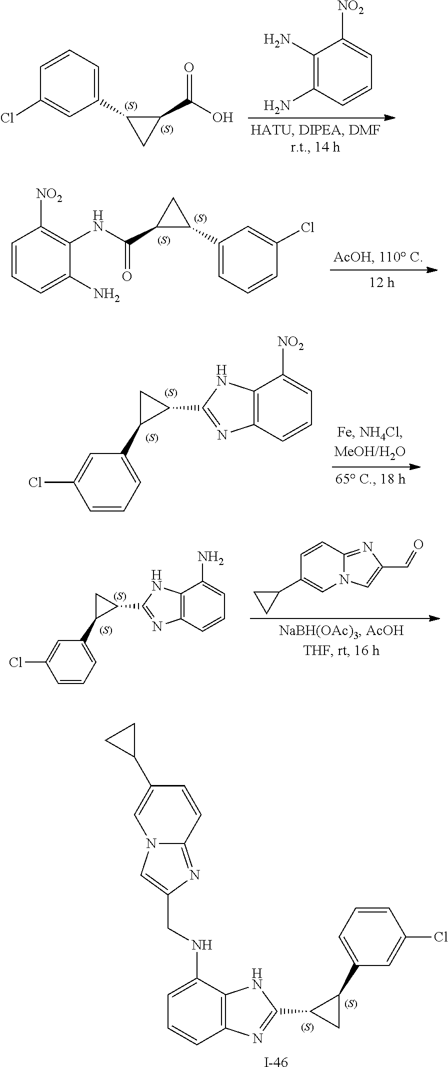

### Synthesis of (1S,2S)—N-(2-amino-6-nitrophenyl)-2-(3-chlorophenyl)cyclopropane-1-carboxamide

To a mixture of (1S,2S)-2-(3-chlorophenyl)cyclopropane-1-carboxylic acid (480 mg, 2.45 mmol) and 3-nitrobenzene-1,2-diamine (306 mg, 2.0 mol) in DMF (20 mL) was added HATU (1.94 g, 5.08 mmol) and DIPEA (1.3 g, 10.15 mmol). The resulting reaction mixture was stirred at room temperature under N₂ for 14 h. The reaction was poured into water (200 mL) and extracted with EtOAc (30 mL×3). The combined organic layers were washed with brine (30 mL), dried over Na₂SO₄, concentrated in vacuo to give the crude, which was purified by column chromatography (eluent: MeOH/DCM=0˜5%) to give (1S,2S)—N-(2-amino-6-nitrophenyl)-2-(3-chlorophenyl)cyclopropane-1-carboxamide (520 mg, 78%) as yellow oil. ESI-MS [M+H]+: 332.1.

### Synthesis of 2-((1S, 2S)-2-(3-chlorophenyl)cyclopropyl)-7-nitro-1H-benzo[d]imidazole

A mixture of (1S,2S)—N-(2-amino-6-nitrophenyl)-2-(3-chlorophenyl)cyclopropane-1-carboxamide (520 mg, 1.57 mmol) in AcOH (20 mL) was stirred at 110° C. for 12 h. The mixture was concentrated in vacuo to remove AcOH, the residue was diluted with NaHCO₃ (sat. aq., 50 mL) and extracted with EtOAc (30 mL×3). The combined organic layers were washed with brine (30 mL), dried over Na₂SO₄, concentrated in vacuo to give the crude, which was purified by column chromatography (eluent: MeOH/DCM=0˜5%) to give the 2-((1S, 2S)-2-(3-chlorophenyl)cyclopropyl)-7-nitro-1H-benzo[d]imidazole (450 mg, 92%) as yellow solid. ESI-MS [M+H]⁺: 314.1.

### Synthesis of 2-((1S, 2S)-2-(3-chlorophenyl)cyclopropyl)-1H-benzo[d]imidazol-7-amine

To a solution of 2-((1S,2S)-2-(3-chlorophenyl)cyclopropyl)-7-nitro-1H-benzo[d]imidazole (375 mg, 1.2 mmol) in MeOH/water (15 mL/5 mL) was added Fe (336 mg, 6.0 mmol) and NH₄Cl (320 mg, 6.0 mmol). The reaction was stirred at 65° C. under N₂ for 18 h. The reaction mixture was filtered through Celite® and the filter cake was washed with DCM/MeOH (10/1, 50 mL). To the filtrate was added water (30 mL) and extracted with DCM/MeOH (10/1, 30 mL×3). The combined organic layers were washed with brine (30 mL), dried over Na₂SO₄ and concentrated in vacuo to give 2-((1S, 2S)-2-(3-chlorophenyl)cyclopropyl)-1H-benzo[d]imidazol-7-amine (320 mg, crude) as brown solid, which was used without further purification. ESI-MS [M+H]⁺: 284.2

### Synthesis of 2-((1S, 2S)-2-(3-chlorophenyl)cyclopropyl)-N-((6-cyclopropylimidazo[1,2-a]pyridin-2-yl)methyl)-1H-benzo[d]imidazol-7-amine

To a mixture of 2-((1S, 2S)-2-(3-chlorophenyl)cyclopropyl)-1H-benzo[d]imidazol-7-amine (120 mg, crude) and 6-cyclopropylimidazo[1,2-a]pyridine-2-carbaldehyde (65 mg, 0.35 mol) in THF (15 mL) was added acetic acid (0.1 mL). The resulting solution was stirred at room temperature for 16 h. NaBH(OAc)₃ (148 mg, 0.7 mmol) was added and the mixture was stirred for an additional hour. The reaction was quenched with NaHCO₃ (sat. aq., 50 mL) and extracted with EtOAc (30 ml×3). The combined organic layers were washed with brine (30 mL), dried over Na₂SO₄ then concentrated in vacuo to give the crude, which was purified with preparative TLC (eluent: DCM/MeOH: 10/1) to give the 2-((1S, 2S)-2-(3-chlorophenyl)cyclopropyl)-N-((6-cyclopropylimidazo[1,2-a]pyridin-2-yl)methyl)-1H-benzo[d]imidazol-7-amine (6.8 mg, 4%) as yellow solid.

ESI-MS [M+H]⁺: 454.1[M+H]⁺, ¹H NMR (400 MHz, DMSO) δ 12.09 (s, 1H), 8.30 (s, 1H), 7.66 (s, 1H), 7.40 (d, J=9.3 Hz, 1H), 7.35-7.31 (m, 2H), 7.25 (d, J=8.6 Hz, 1H), 7.20 (d, J=7.7 Hz, 1H), 6.97-6.95 (m, 1H), 6.86 (t, J=7.8 Hz, 1H), 6.64 (s, 1H), 6.27 (s, 1H), 5.70-5.67 (m, 1H), 4.48 (d, J=5.4 Hz, 2H), 2.48-2.45 (m, 1H), 2.41-2.36 (m, 1H), 1.94-1.87 (m, 1H), 1.79 (s, 1H), 1.64-1.59 (m, 1H), 0.92-0.88 (m, 2H), 0.68-0.64 (m, 2H).

### Example 47

### Synthesis of rac-tert-butyl (2-((1S*,2S*)-2-(3-chlorophenyl)cyclopropyl)-4-methoxyquinolin-7-yl)((6-cyclopropylimidazo[1,2-a]pyridin-2-yl)methyl)carbamate (I-47)

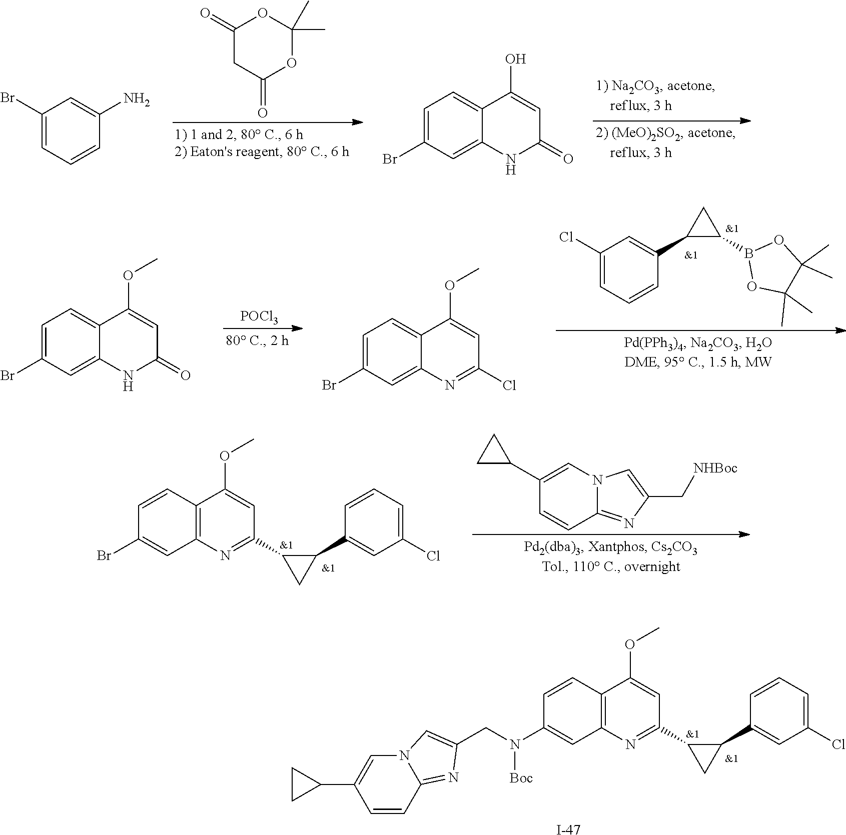

### Synthesis of rac-2-((1S*,2S*)-2-(3-chlorophenyl)cyclopropyl)-4,4,5,5-tetramethyl-1,3,2-dioxaborolane

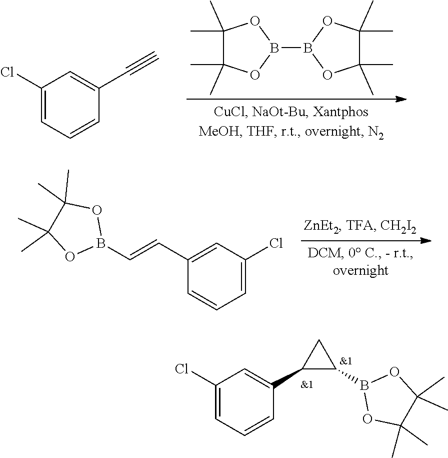

### Synthesis of (E)-2-(3-chlorostyryl)-4,4,5,5-tetramethyl-1,3,2-dioxaborolane

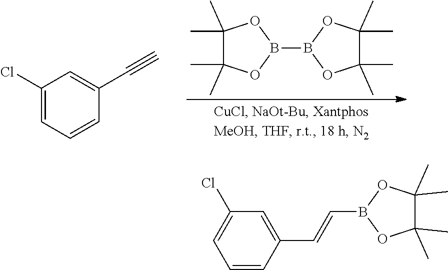

CuCl (287 mg, 2.9 mmol), t-BuONa (557 mg, 5.8 mmol), and Xantphos (1.7 g, 2.9 mmol) were placed in an oven-dried Schlenk tube under nitrogen and THF (60 mL) was added. The reaction mixture was stirred for 30 min at room temperature and then bis(pinacolato)diboron (7.4 g, 29 mmol) in THF (30 mL) was added. The reaction mixture was stirred for 10 min and 1-chloro-3-ethynylbenzene (4 g, 29 mmol) was added, followed by MeOH (1.8 g, 58 mmol). The mixture was stirred at room temperature for 18 h. After completed, the reaction mixture was quenched with water (80 mL), extracted with EtOAc (80 mL×3). The combined organic layers were washed with brine (80 mL), dried over anhydrous Na₂SO₄, filtered and concentrated in vacuo. The reside was purified by column chromatography on silica gel (eluent: EtOAc/PE=0-3%) to afford (E)-2-(3-chlorostyryl)-4,4,5,5-tetramethyl-1,3,2-dioxaborolane (4.4 g, 57%) as pale-yellow sticky oil. ESI-MS [M+H]+: 265.1.

### Synthesis of rac-2-((1S*,2S*)-2-(3-chlorophenyl)cyclopropyl)-4,4,5,5-tetramethyl-1,3,2-dioxaborolane

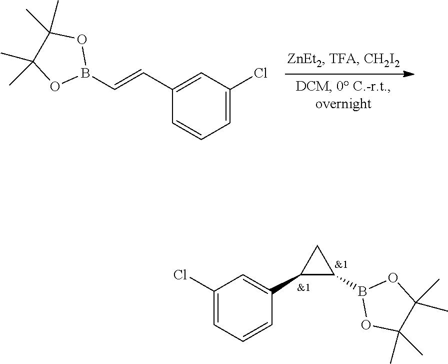 [SMILES: CC1(C)OB(/C=C/c2cccc(Cl)c2)OC1(C)C ; CC1(C)OB([C@H]2C[C@@H]2c2cccc(Cl)c2)OC1(C)C]

To a solution of ZnEt₂ (20 mL, 20 mmol, 1 M in hexane) in dry DCM (25 mL) was added (very slowly) a solution of trifluoroacetic acid (2.3 g, 20 mmol) in DCM (15 mL) at 0° C. After the addition (25 minutes), the reaction mixture was stirred for another 30 minutes at 0° C. Then a solution of diiodomethane (5.4 g, 20 mmol) in DCM (15 mL) was added thereto. After stirred at additional 30 minutes at 0° C., a solution of (E)-2-(3-chlorostyryl)-4,4,5,5-tetramethyl-1,3,2-dioxaborolane (2.7 g, 10 mmol) in DCM (15 mL) was added to the reaction above and the reaction mixture was allowed to warm to room temperature and stirred overnight. The reaction was quenched with NH₄Cl (Sat. aq., 50 mL) and extracted with DCM (50 mL×3). The organic layer was dried over anhydrous Na₂SO₄ then concentrated in vacuo. The residue was purified by column chromatography on silica gel (eluent: PE/EtOAc=20:1) to afford 2-(2-(3-chlorophenyl)cyclopropyl)-4,4,5,5-tetramethyl-1,3,2-dioxaborolane (2.1 g, yield 75%) as colorless oil.

ESI-MS [M+H]+: 279.1, ¹H NMR (400 MHz, DMSO) δ 7.27-7.23 (m, 1H), 7.19-7.16 (m, 2H), 7.06-7.04 (m, 1H), 2.04-2.00 (m, 1H), 1.20-1.15 (m, 12H), 1.07-1.01 (m, 2H), 0.23-0.18 (m, 1H).

### Synthesis of 7-bromo-4-hydroxyquinolin-2 (1H)-one and 5-bromo-4-hydroxyquinolin-2 (1H)-one

In an oven dried 100 mL round bottom flask, 3-bromoaniline (10 g, 58 mmol) and 2,2-dimethyl-1,3-dioxane-4,6-dione (10 g, 69 mmol) were added and heated at 80° C. for 6 h. After consumption of starting material (TLC), the reaction was diluted with NaHCO₃(Sat. aq., 50 mL), washed with EtOAc (100 mL×3). The organic layer was discarded. The pH of aqueous layer was adjusted to pH=1 with concentrated HCl then extracted with DCM (75 mL×3). The combined organic layers were washed with brine (70 mL), dried over anhydrous Na₂SO₄ and concentrated in vacuo to give the crude which was treated with Eaton's reagent (80 g) at 80° C. for 6 h. After completed, the reaction mixture was cooled to 0° C., quenched with cold water (100 mL) and stirred for 30 min until the solid was precipitated. The mixture was filtered, and the filter cake was dried to give 7-bromo-4-hydroxyquinolin-2 (1H)-one (6.5 g crude, mixed with 5-bromo-4-hydroxyquinolin-2 (1H)-one) as a white solid. ESI-MS: [M+H]⁺, 240.0

### Synthesis of 7-bromo-4-methoxyquinolin-2 (1H)-one

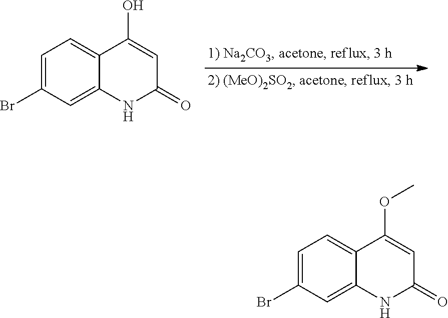

A mixture of 7-bromo-4-hydroxyquinolin-2 (1H)-one (6.5 g crude, mixed with 5-bromo-4-hydroxyquinolin-2 (1H)-one) and K₂CO₃ (7.5 g, 54 mmol) in acetone (120 mL) was refluxed for 3 h. The reaction was cooled to room temperature, dimethylsulfate (3.4 g, 27 mmol) was added, and the mixture was refluxed for another 3 h. The reaction was concentrated in vacuo to remove the solvent, the residue was diluted with H₂O (80 mL), extracted with EtOAc (80 mL×3). The combined organic layers were washed with brine (80 mL), dried over anhydrous Na₂SO₄ then concentrated in vacuo to give 7-bromo-4-methoxyquinolin-2 (1H)-one (3.3 g crude), which was used without further purification.

ESI-MS: [M+H]⁺, 254.0

### Synthesis of 7-bromo-2-chloro-4-methoxyquinoline and 5-bromo-2-chloro-4-methoxyquinoline

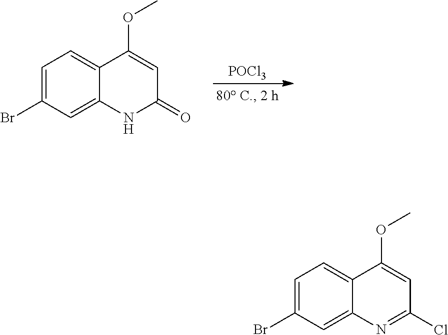

A mixture of 7-bromo-4-methoxyquinolin-2 (1H)-one (2.4 g, crude) and POCl₃ (7.2 g, 47 mmol) was stirred at 80° C. for 2 h. After the removal of excess POCl₃, the dark brown liquid was added to cold water (150 mL), and neutralized with NaHCO₃(Sat. aq., 50 mL), followed by extraction with EtOAc (100 mL×3). The combined organic layers were washed with brine (100 mL), dried over anhydrous Na₂SO₄, and concentrated in vacuo to give a white solid, which was purified by Prep-HPLC (Chromatographic columns: Kromasil-C18 100×21.2 mm×5 um, Mobile Phase: ACN-H₂O (0.1% FA), Gradient: 60-70) to afford 7-bromo-2-chloro-4-methoxyquinoline (640 mg, 6% for 3 steps) as white solid as a white solid.

ESI-MS: [M+H]+, 272.0, ¹H NMR (400 MHz, DMSO) δ 8.11 (d, J=1.9 Hz, 1H), 8.04 (d, J=8.9 Hz, 1H), 7.74 (dd, J=8.9, 2.0 Hz, 1H), 7.17 (s, 1H), 4.09 (s, 3H).

### Synthesis of rac-7-bromo-2-((1S*,2S*)-2-(3-chlorophenyl)cyclopropyl)-4-methoxyquinoline

A mixture of 7-bromo-2-chloro-4-methoxyquinoline (300 mg, 1.1 mmol), Pd(PPh₃)₄ (380 mg, 0.33 mmol), 2-(2-(3-chlorophenyl) cyclopropyl)-4,4,5,5-tetramethyl-1,3,2-dioxaborolane (473 mg, 1.7 mmol), and Na₂CO₃ (350 mg, 3.3 mmol) in DME (8 mL) and H₂O (2 mL) was stirred in a sealed tube and irradiated in microwave at 95° C. for 1.5 h. The mixture was diluted with water (60 mL) and extracted with DCM (50 mL×3). The combined organic layers were washed by brine (80 mL), dried over Na₂SO₄, and concentrated in vacuo to give the crude, which was purified by column chromatography on silica gel (eluent: EA/PE=1/15) to afford 7-bromo-2-(2-(3-chlorophenyl) cyclopropyl)-4-methoxyquinoline (90 mg, yield 21%) as colorless oil.

ESI-MS: [M+H]+, 388.0, ¹H NMR (400 MHz, DMSO) δ 8.01-7.98 (m, 2H), 7.59 (dd, J=8.8, 2.0 Hz, 1H), 7.34-7.13 (m, 5H), 4.04 (s, 3H), 2.64-2.57 (m, 2H), 1.92-1.88 (m, 1H), 1.64-1.59 (m, 1H).

### Synthesis of rac-tert-butyl (2-((1S*,2S*)-2-(3-chlorophenyl)cyclopropyl)-4-methoxyquinolin-7-yl)((6-cyclopropylimidazo[1,2-a]pyridin-2-yl)methyl)carbamate

A mixture of tert-butyl ((6-cyclopropylimidazo[1,2-a]pyridin-2-yl)methyl)carbamate (59 mg, 0.21 mmol), 7-bromo-2-(2-(3-chlorophenyl)cyclopropyl)-4-methoxyquinoline (80 mg, 0.21 mmol), Pd₂(dba)₃ (38 mg, 0.041 mmol), Xantphos (47 mg, 0.082 mmol) and Cs₂CO₃ (200 mg, 0.62 mmol) in toluene (8 mL) was stirred at 110° C. overnight under N₂. The reaction was filtered and concentrated in vacuo. The residue was diluted with water (30 mL), and then extracted with EtOAc (30 mL×3). The organic layers were dried over Na₂SO₄ and concentrated in vacuo. The residue was purified by preparative TLC (eluent: DCM/MeOH=20/1) to afford rac-tert-butyl (2-((1S*,2S*)-2-(3-chlorophenyl)cyclopropyl)-4-methoxyquinolin-7-yl)((6-cyclopropylimidazo[1,2-a]pyridin-2-yl)methyl)carbamate (20 mg, yield 16%) as a white solid.

ESI-MS [M+H]+: 595.3. ¹H NMR (400 MHz, MeOD) δ 8.14 (s, 1H), 8.04 (d, J=8.9 Hz, 1H), 7.72 (d, J=1.8 Hz, 1H), 7.64 (s, 1H), 7.42-7.39 (m, 1H), 7.35 (d, J=9.3 Hz, 1H), 7.27-7.23 (m, 1H), 7.20-7.16 (m, 2H), 7.12 (d, J=7.7 Hz, 1H), 7.04 (dd, J=9.4, 1.6 Hz, 1H), 6.79 (s, 1H), 5.06 (s, 2H), 4.05 (s, 3H), 2.61-2.56 (m, 1H), 2.49-2.45 (m, 1H), 1.92-1.83 (m, 2H), 1.57-1.53 (m, 1H), 1.46 (s, 9H), 0.97-0.92 (m, 2H), 0.69-0.67 (m, 2H).

### Example 48

### Synthesis of rac-2-((1S*,2S*)-2-(3-chlorophenyl)cyclopropyl)-N-((6-cyclopropylimidazo[1,2-a]pyridin-2-yl)methyl)-4-methoxyquinolin-7-amine (1-48)

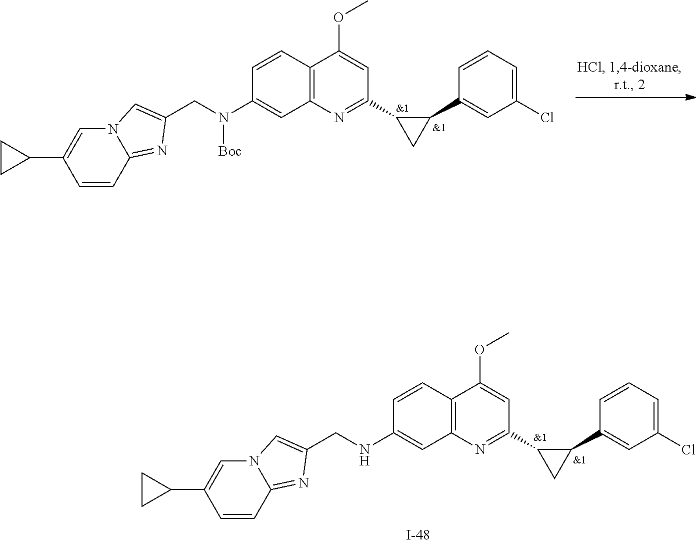

To a solution of rac-tert-butyl (2-((1S*,2S*)-2-(3-chlorophenyl)cyclopropyl)-4-methoxyquinolin-7-yl)((6-cyclopropylimidazo[1,2-a]pyridin-2-yl)methyl)carbamate (10 mg, 0.017 mmol) in 1,4-dioxane (1 mL) was added HCl (0.2 mL, 0.8 mmol, 4 M in 1,4-dioxane). The mixture was stirred at room temperature for 2 h. The reaction was concentrated in vacuo. The residue was purified by preparative TLC (eluent: DCM/MeOH=10/1) to afford rac-2-((1S*,2S*)-2-(3-chlorophenyl)cyclopropyl)-N-((6-cyclopropylimidazo[1,2-a]pyridin-2-yl)methyl)-4-methoxyquinolin-7-amine (4 mg, yield 48%) as a white solid.

ESI-MS [M+H]+: 495.2. ¹H NMR (400 MHz, MeOD) δ 8.12 (s, 1H), 7.86 (d, J=9.0 Hz, 1H), 7.64 (s, 1H), 7.38 (d, J=9.3 Hz, 1H), 7.27-7.16 (m, 3H), 7.13-7.11 (m, 1H), 7.06 (dd, J=9.3, 1.6 Hz, 1H), 6.98-6.95 (m, 1H), 6.85 (d, J=2.1 Hz, 1H), 6.51 (s, 1H), 4.61 (s, 2H), 4.03 (s, 3H), 2.57-2.52 (m, 1H), 2.44-2.39 (m, 1H), 2.05-2.00 (m, 1H), 1.84-1.79 (m, 1H), 1.57-1.52 (m, 1H), 0.97-0.92 (m, 2H), 0.71-0.67 (m, 2H)

### Example 49

### Synthesis of rac-2-((1S*,2S*)-2-(3-chlorophenyl)cyclopropyl)-7-(((6-cyclopropylimidazo[1,2-a]pyridin-2-yl)methyl)amino)quinolin-4 (1H)-one (I-49)

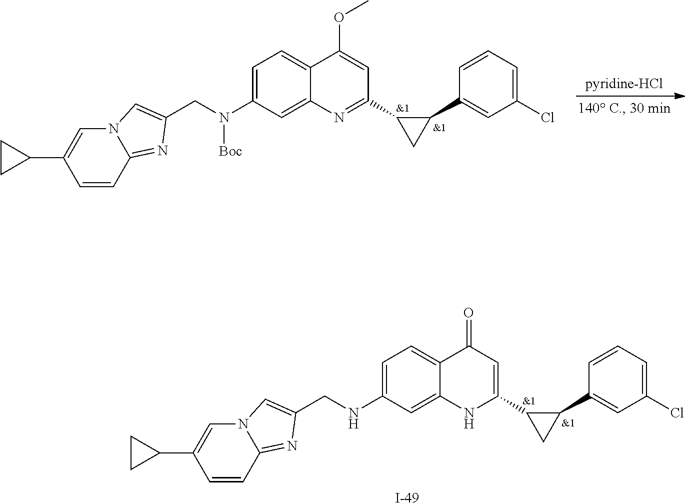

A mixture of rac-tert-butyl (2-((1S*,2S*)-2-(3-chlorophenyl)cyclopropyl)-4-methoxyquinolin-7-yl)((6-cyclopropylimidazo[1,2-a]pyridin-2-yl)methyl)carbamate (25 mg, 0.042 mmol) and pyridine-HCl (0.10 g) was heated to 140° C. for 30 min. The resulting mixture was quenched with H₂O (20 mL), and then extracted with EtOAc (25 mL×3). The combined organic layers were washed with brine (30 mL), dried over anhydrous Na₂SO₄, and then concentrated in vacuo to give the crude, which was purified by preparative TLC, eluting with 10% MeOH/DCM to give rac-2-((1S*,2S*)-2-(3-chlorophenyl)cyclopropyl)-7-(((6-cyclopropylimidazo[1,2-a]pyridin-2-yl)methyl)amino)quinolin-4 (1H)-one (12 mg, 60%) as a white solid.

ESI-MS [M+H]⁺: 481.1, ¹H NMR (400 MHz, DMSO) δ 11.02 (s, 1H), 8.29 (s, 1H), 7.70 (d, J=8.8 Hz, 1H), 7.61 (s, 1H), 7.39-7.16 (m, 5H), 6.96 (d, J=9.4 Hz, 1H), 6.90 (s, 1H), 6.67 (d, J=8.6 Hz, 1H), 6.40 (s, 1H), 5.57 (s, 1H), 4.39 (d, J=5.5 Hz, 2H), 2.37-2.33 (m, 1H), 2.14-2.08 (m, 1H), 1.93-1.86 (m, 1H), 1.66-1.62 (m, 1H), 1.52-1.48 (m, 1H), 0.92-0.84 (m, 2H), 0.66-0.63 (m, 2H).

### Example 50

### Synthesis of 2-((1S,2S)-2-(3-chlorophenyl)cyclopropyl)-7-(((6-cyclopropylimidazo[1,2-a]pyridin-2-yl)methyl)amino)quinazolin-4 (1H)-one (I-50)

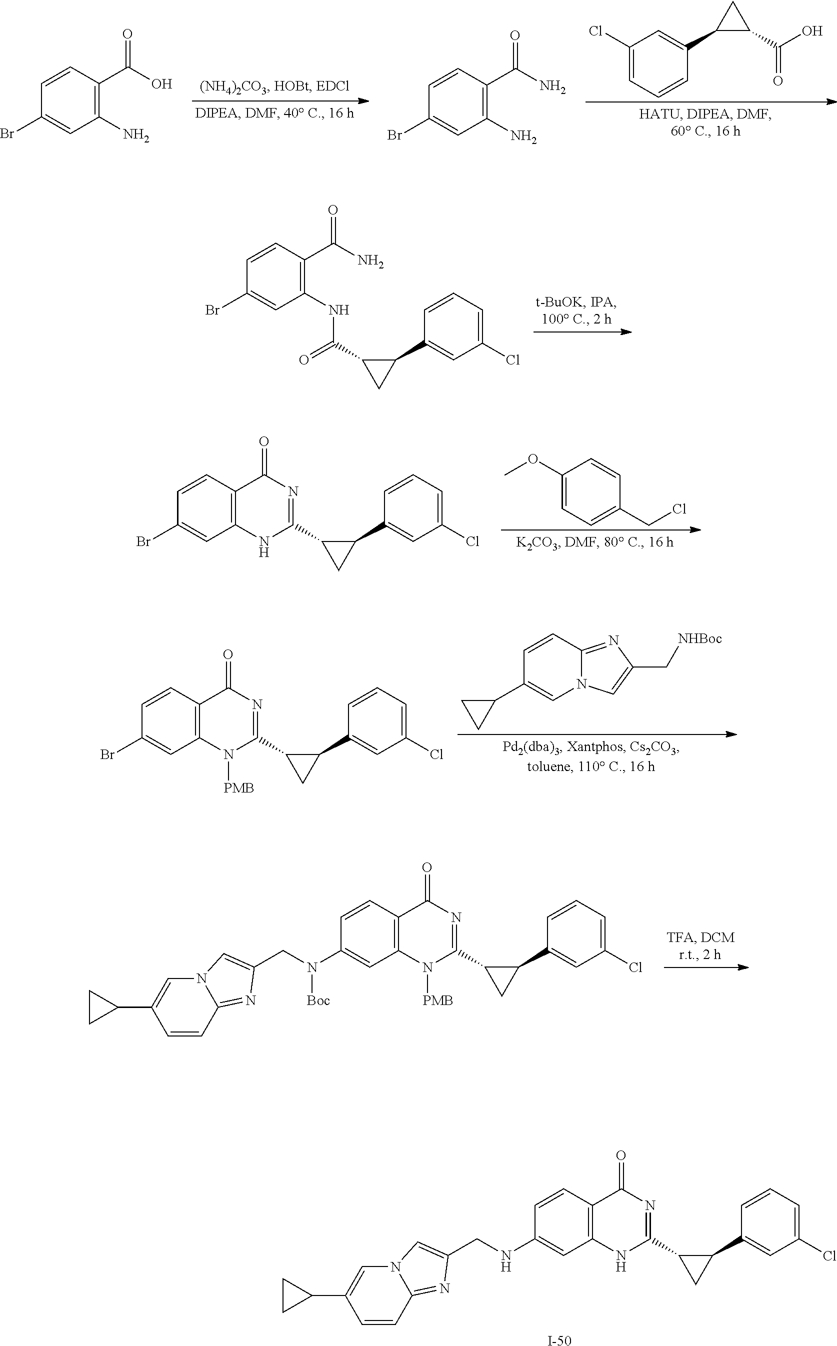

### Synthesis of 2-amino-4-bromobenzamide

To a solution of 2-amino-4-bromobenzoic acid (4 g, 18.6 mmol) in DMF (80 mL) was added (NH₄)₂CO₃ (8.88 g, 92.5 mmol), HOBt (3.64 g, 27 mmol), EDCI (5.18 g, 27 mmol) and DIPEA (13.9 g, 108 mmol). Then the mixture was stirred at 40° C. for 16 h. The reaction was cooled to room temperature, diluted with water (100 mL) and extracted with EtOAc (100 mL×3). The combined organic layers were washed with brine (80 mL), dried over Na₂SO₄, concentrated in vacuo to give the crude, which was purified by silica gel column chromatography eluting with a gradient of 0-30% EtOAc in PE to give 2-amino-4-bromobenzamide (3.2 g, 80%) as a white solid. ESI-MS [M+H]+: 215.1.

### Synthesis of 4-bromo-2-((1S,2S)-2-(3-chlorophenyl)cyclopropane-1-carboxamido)benzamide

To a solution of 2-amino-4-bromobenzamide (2 g, 9.3 mmol) in DMF (40 mL) was added (1S,2S)-2-(3-chlorophenyl)cyclopropane-1-carboxylic acid (1.82 g, 9.3 mmol), HATU (7.06 g, 18.6 mmol) and DIPEA (3.6 g, 27.9 mmol). Then the reaction mixture was stirred at 60° C. for 16 h. The reaction was cooled to room temperature, water (100 mL) was added and extracted with EtOAc (100 mL×3). The combined organic layers were washed with brine (70 mL), dried over Na₂SO₄, concentrated in vacuo and purified by silica gel column chromatography eluting with a gradient of 0-35% EtOAc in PE to give 4-bromo-2-((1S,2S)-2-(3-chlorophenyl)cyclopropane-1-carboxamido)benzamide (2.1 g, 58%) as a white solid. ESI-MS [M+H]⁺: 394.1 Synthesis of 7-bromo-2-((1S,2S)-2-(3-chlorophenyl)cyclopropyl)quinazolin-4 (1H)-one

A solution of 4-bromo-2-((1S,2S)-2-(3-chlorophenyl)cyclopropane-1-carboxamido)benzamide (2 g, 5.1 mmol) and t-BuOK (1.7 g, 15.2 mmol) in i-PrOH (50 mL) was stirred at 100° C. for 2 h. The reaction was cooled to room temperature and concentrated in vacuo, the residue was diluted with water (100 mL) and extracted with EtOAc (50 mL×3). The combined organic layers were washed with brine (80 mL), dried over anhydrous Na₂SO₄, concentrated in vacuo to give the crude, which was purified by silica gel column chromatography eluting with a gradient 0-50% EtOAc in PE to give 7-bromo-2-((1S,2S)-2-(3-chlorophenyl)cyclopropyl)quinazolin-4 (1H)-one (1.5 g, 79%) as a white solid. ESI-MS [M+H]+: 375.1.

### Synthesis of 7-bromo-2-((1S,2S)-2-(3-chlorophenyl)cyclopropyl)-1-(4-methoxybenzyl)quinazolin-4 (1H)-one

A solution of 7-bromo-2-((1S,2S)-2-(3-chlorophenyl)cyclopropyl)quinazolin-4 (1H)-one (200 mg, 0.53 mmol), 1-(chloromethyl)-4-methoxybenzene (91 mg, 0.58 mmol) and K₂CO₃ (183 mg, 1.32 mmol) in DMF (10 mL) was stirred at 80° C. for 16 h. The reaction was cooled to room temperature, diluted with water (30 mL) and extracted with EtOAc (30 mL×3). The combined organic layers were washed with brine (40 mL), dried over anhydrous Na₂SO₄, concentrated in vacuo to give the crude, which was purified by silica gel column chromatography eluting with a gradient of 0-50% EtOAc in PE to give 7-bromo-2-((1S,2S)-2-(3-chlorophenyl)cyclopropyl)-1-(4-methoxybenzyl)quinazolin-4 (1H)-one (110 mg, 42%) as a white solid. ESI-MS [M+H]⁺: 495.1.

### Synthesis of tert-butyl (2-((1S,2S)-2-(3-chlorophenyl)cyclopropyl)-1-(4-methoxybenzyl)-4-oxo-1,4-dihydroquinazolin-7-yl)((6-cyclopropylimidazo[1,2-a]pyridin-2-yl)methyl)carbamate

To a mixture of 7-bromo-2-((1S,2S)-2-(3-chlorophenyl)cyclopropyl)-1-(4-methoxybenzyl)quinazolin-4 (1H)-one (100 mg, 0.2 mmol), tert-butyl ((6-cyclopropylimidazo[1,2-a]pyridin-2-yl)methyl)carbamate (64 mg, 0.22 mmol) and Cs₂CO₃ (196 mg, 0.6 mmol) in toluene (5 mL) was added Pd₂(dba)₃ (27 mg, 0.03 mmol) and Xantphos (35 mg, 0.06 mmol). The reaction mixture was stirred at 110° C. for 16 h under N₂. The reaction mixture was filtered through Celite® and the filter cake was washed with DCM/MeOH (10/1, 20 mL). The filtrate was concentrated in vacuo to give the crude, which was purified by preparative TLC (eluent: DCM/MeOH=50/1) to give tert-butyl (2-((1S,2S)-2-(3-chlorophenyl)cyclopropyl)-1-(4-methoxybenzyl)-4-oxo-1,4-dihydroquinazolin-7-yl)((6-cyclopropylimidazo[1,2-a]pyridin-2-yl)methyl)carbamate (40 mg, 28%) as yellow oil. ESI-MS [M+H]+: 702.1.

### Synthesis of 2-((1S,2S)-2-(3-chlorophenyl)cyclopropyl)-7-(((6-cyclopropylimidazo[1,2-a]pyridin-2-yl)methyl)amino)quinazolin-4 (1H)-one

To a solution of tert-butyl (2-((1S,2S)-2-(3-chlorophenyl)cyclopropyl)-1-(4-methoxybenzyl)-4-oxo-1,4-dihydroquinazolin-7-yl)((6-cyclopropylimidazo[1,2-a]pyridin-2-yl)methyl)carbamate (40 mg, 0.057 mmol) in DCM (5 mL) was added TFA (2 mL). Then the rection mixture was stirred at room temperature for 2 h. The reaction was concentrated in vacuo to give the residue, which was washed with NaHCO₃ (sat. aq., 30 mL) and extracted with DCM (30 mL×3). The combined organic layers were washed with brine (30 mL), dried over anhydrous Na₂SO₄ then concentrated in vacuo. The crude was purified by preparative TLC (eluent: DCM/MeOH=15/1) to give 2-((1S,2S)-2-(3-chlorophenyl)cyclopropyl)-7-(((6-cyclopropylimidazo[1,2-a]pyridin-2-yl)methyl)amino)quinazolin-4 (1H)-one (17 mg, 62%) as a white solid.

ESI-MS [M+H]+: 482.1. ¹H NMR (400 MHz, DMSO) δ 11.86 (s, 1H), 8.31 (s, 1H), 7.72 (d, J=8.8 Hz, 1H), 7.65 (s, 1H), 7.40 (d, J=9.3 Hz, 1H), 7.33-7.24 (m, 3H), 7.20-7.05 (m, 2H), 6.97 (d, J=9.3 Hz, 1H), 6.79 (d, J=8.7 Hz, 1H), 6.46 (s, 1H), 4.43 (d, J=5.5 Hz, 2H), 2.56-2.52 (m, 1H), 2.19-2.16 (m, 1H), 1.96-1.85 (m, 1H), 1.77-1.72 (m, 1H), 1.56-1.51 (m, 1H), 0.98-0.82 (m, 2H), 0.68-0.64 (m, 2H).

### Example 51

### Synthesis of 2-((1S,2S)-2-(3-chlorophenyl)cyclopropyl)-7-(((6-cyclopropylimidazo[1,2-a]pyridin-2-yl)methyl)amino)quinolin-4-ol (I-51)

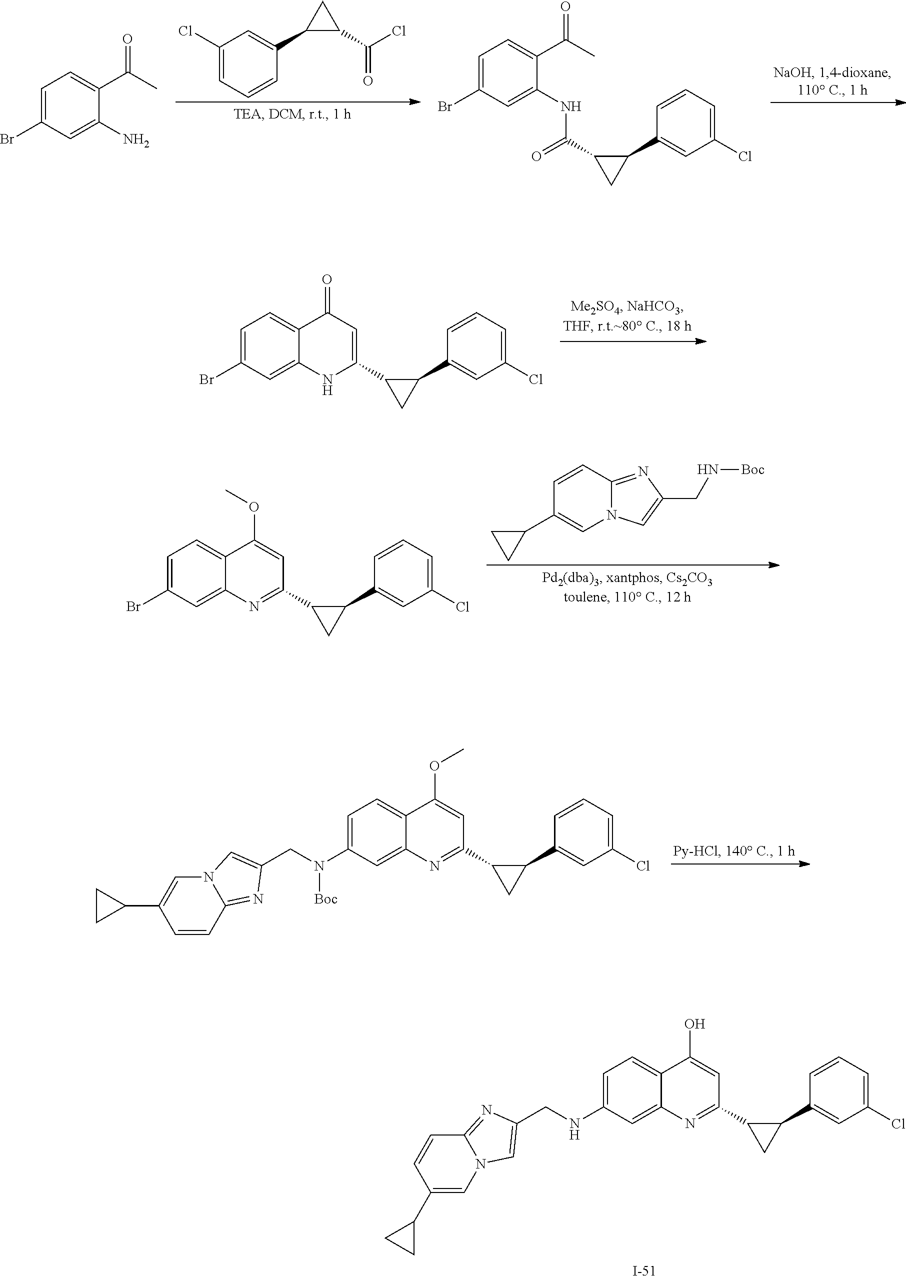

### Synthesis of (1S,2S)—N-(2-acetyl-5-bromophenyl)-2-(3-chlorophenyl)cyclopropane-1-carboxamide

To a mixture of 1-(2-amino-4-bromophenyl)ethan-1-one (600 mg, 2.8 mmol) and TEA (848 mg, 8.4 mmol) in DCM (12 mL) was added a solution of (1S,2S)-2-(3-chlorophenyl)cyclopropane-1-carbonyl chloride (731 mg, 3.4 mmol) in DCM (3 mL) at 0° C. The resulting reaction was attired at room temperature for 1 h. The reaction was quenched with water (30 mL), extracted with DCM (20 mL×3). The combined organic layers were washed with brine (30 mL), dried over Na₂SO₄, filtered and concentrated in vacuo to give the crude, which was purified by silica gel column chromatography (eluent: DCM/MeOH=100/1˜40/1) to give (1S,2S)—N-(2-acetyl-5-bromophenyl)-2-(3-chlorophenyl)cyclopropane-1-carboxamide (900 mg, 82%) as a white solid. ESI-MS [M+H]⁺: 392.2.

### Synthesis of 7-bromo-2-((1S,2S)-2-(3-chlorophenyl)cyclopropyl)quinolin-4 (1H)-one

To a solution of (1S,2S)—N-(2-acetyl-5-bromophenyl)-2-(3-chlorophenyl)cyclopropane-1-carboxamide (900 mg, 2.3 mmol) in 1,4-dioxane (20 mL) and was added NaOH (276 mg, 6.9 mmol). The reaction mixture was stirred at 110° C. for 1 h. The reaction was cooled to room temperature, diluted with water (30 mL) then extracted with EtOAc (40 mL×3). The combined organic layers were washed with brine (20 mL), dried over anhydrous Na₂SO₄, and concentrated in vacuo to give the crude product, which was purified by silica gel column chromatography (eluent: DCM/MeOH=100/1˜40/1) to give 7-bromo-2-((1S,2S)-2-(3-chlorophenyl)cyclopropyl)quinolin-4 (1H)-one (700 mg, 82%) as a yellow solid. ESI-MS [M+H]+: 374.2.

### Synthesis of 7-bromo-2-((1S,2S)-2-(3-chlorophenyl)cyclopropyl)quinolin-4(H)-one

To a solution of 7-bromo-2-((1S,2S)-2-(3-chlorophenyl)cyclopropyl)quinolin-4 (1H)-one (700 mg, 1.87 mmol) in THF (10 mL) and was added NaHCO₃ (314 mg, 3.74 mmol) and Me₂SO₄ (235 mg, 1.87 mmol). The reaction mixture was stirred at 80° C. for 18 h. The reaction was diluted with water (30 mL) then extracted with EtOAc (30 mL×3). The combined organic layers were washed with brine (30 mL), dried over anhydrous Na₂SO₄, and concentrated in vacuo to give the crude product, which was purified by silica gel column chromatography (eluent: DCM/MeOH=100/1˜50/1) to give 7-bromo-2-((1S,2S)-2-(3-chlorophenyl)cyclopropyl)-4-methoxyquinoline (550 mg, 76%) as a yellow solid. ESI-MS [M+H]+: 388.2.

### Synthesis of tert-butyl (2-((1S,2S)-2-(3-chlorophenyl)cyclopropyl)-4-methoxyquinolin-7-yl)((6-cyclopropylimidazo[1,2-a]pyridin-2-yl)methyl)carbamate

To a mixture of 7-bromo-2-((1S,2S)-2-(3-chlorophenyl)cyclopropyl)-4-methoxyquinoline (90 mg, 0.23 mmol), tert-butyl ((6-cyclopropylimidazo[1,2-a]pyridin-2-yl)methyl)carbamate (66 mg, 0.23 mmol) and Cs₂CO₃ (225 mg, 0.69 mmol) in toluene (5 mL) was added Pd₂(dba)₃ (31 mg, 0.034 mmol) and Xantphos (27 mg, 0.046 mmol). The reaction mixture was stirred at 110° C. for 12 h under N₂. The reaction mixture was cooled to room temperature, diluted with DCM (20 mL), filtered through Celite® and the filter cake was washed with DCM/MeOH (20/1, 50 mL). The filtrate was concentrated in vacuo to give the crude, which was purified by preparative TLC (DCM/MeOH=20/1) to give tert-butyl (2-((1S,2S)-2-(3-chlorophenyl)cyclopropyl)-4-methoxyquinolin-7-yl)((6-cyclopropylimidazo[1,2-a]pyridin-2-yl)methyl)carbamate (28 mg, 20%) as a yellow solid. ESI-MS [M+H]⁺: 595.2.

### Synthesis of 2-((1S,2S)-2-(3-chlorophenyl)cyclopropyl)-7-(((6-cyclopropylimidazo[1,2-a]pyridin-2-yl)methyl)amino)quinolin-4-ol

A mixture of tert-butyl (2-((1S,2S)-2-(3-chlorophenyl)cyclopropyl)-4-methoxyquinolin-7-yl)((6-cyclopropylimidazo[1,2-a]pyridin-2-yl)methyl)carbamate (28 mg, 0.047 mmol) and pyridine hydrochloride (54 mg, 0.47 mmol) was stirred at 140° C. for 1 h under N₂. The reaction was quenched with water (20 mL), extracted with EtOAc (20 mL×3). The combined organic layers were washed with brine (20 mL), dried over anhydrous Na₂SO₄, and concentrated in vacuo to give the crude product, which was purified by preparative TLC (DCM/MeOH=20/1) to give 2-((1S,2S)-2-(3-chlorophenyl)cyclopropyl)-7-(((6-cyclopropylimidazo[1,2-a]pyridin-2-yl)methyl)amino)quinolin-4-ol (8 mg, 35%) as a white solid.

ESI-MS [M+H]+: 481.1. ¹H NMR (400 MHz, DMSO) δ 11.02 (s, 1H), 8.29 (s, 1H), 7.70 (d, J=8.8 Hz, 1H), 7.62 (s, 1H), 7.39 (d, J=9.1 Hz, 1H), 7.30-7.26 (m, 3H), 7.17 (d, J=7.6 Hz, 1H), 6.99-6.93 (m, 2H), 6.70-6.65 (m, 1H), 6.41 (s, 1H), 5.57 (s, 1H), 4.40 (d, J=5.5 Hz, 2H), 2.39-2.29 (m, 1H), 2.17-2.10 (m, 1H), 1.99-1.85 (m, 1H), 1.68-1.61 (m, 1H), 1.55-1.47 (m, 1H), 0.93-0.86 (m, 2H), 0.68-0.61 (m, 2H).

### Example 52

### Synthesis of 7-((2R,4S)-4-((tert-butyldimethylsilyl)oxy)-2-(6-cyclopropylimidazo[1,2-a]pyridin-2-yl)pyrrolidin-1-yl)-4-methoxy-2-((1RS,2RS)-2-(4-methylpyrimidin-2-yl)cyclopropyl)quinoline (I-52)

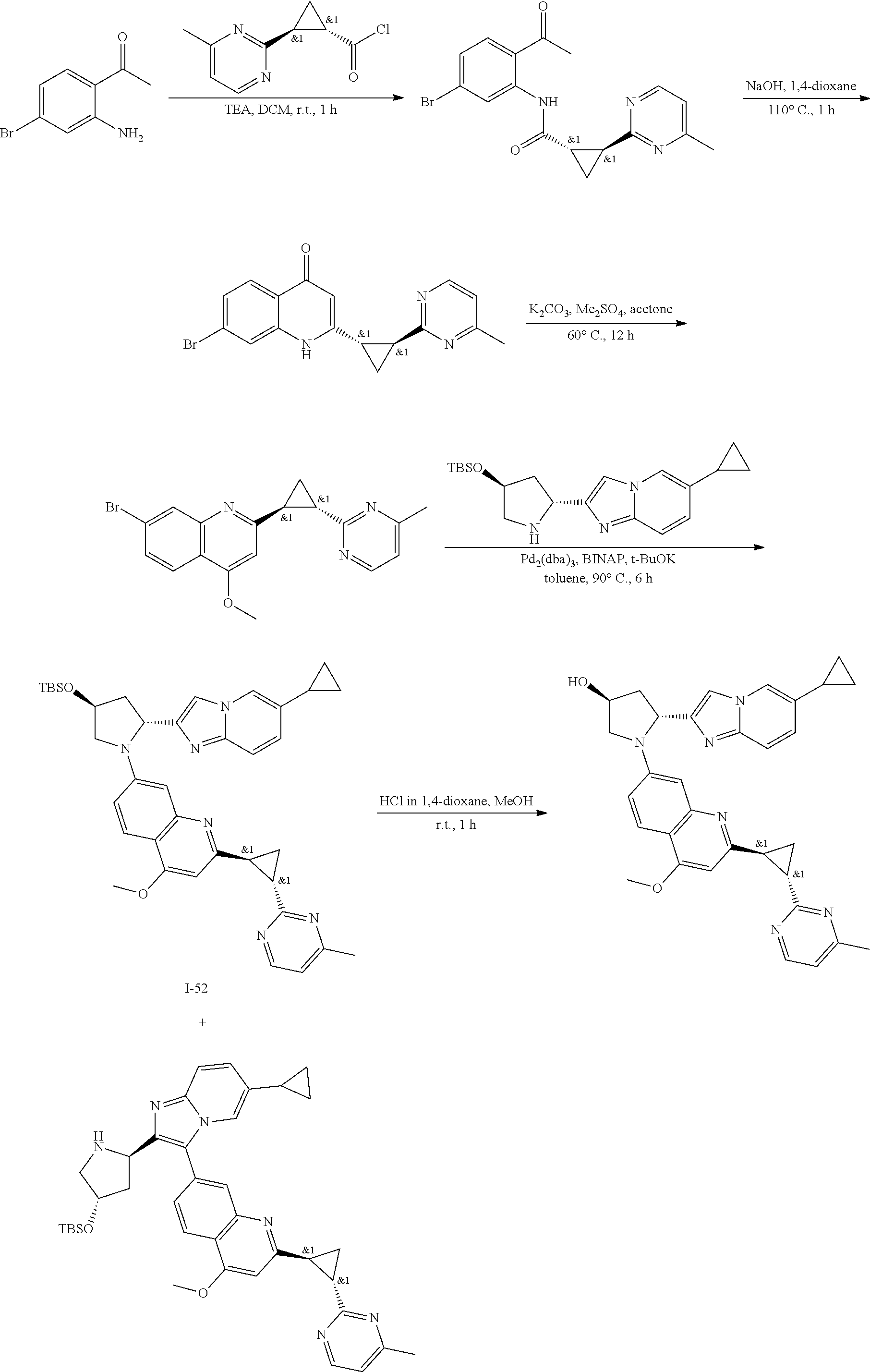

### Synthesis of rac-(1S*,2S*)—N-(2-acetyl-5-bromophenyl)-2-(4-methylpyrimidin-2-yl)cyclopropane-1-carboxamide

To a stirred solution of 1-(2-amino-4-bromophenyl)ethan-1-one (556 mg, 2.6 mmol) and TEA (787.8 mg, 7.8 mmol) in DCM (10 mL) was added dropwise a solution of rac-(1S*,2S*)-2-(4-methylpyrimidin-2-yl)cyclopropane-1-carbonyl chloride (607 mg, 3.1 mmol) in DCM (10 mL) at 0° C. The mixture was stirred at room temperature for 12 h. The reaction mixture was diluted in DCM (30 mL), washed with water (40 mL) and brine (40 mL), dried over Na₂SO₄, and then concentrated in vacuo. The residue was purified by column chromatography on silica gel, eluting with 10˜30% EtOAc/PE to give rac-(1S*,2S*)—N-(2-acetyl-5-bromophenyl)-2-(4-methylpyrimidin-2-yl)cyclopropane-1-carboxamide (0.78 g, 80%) as a pale solid. ESI-MS [M+H]⁺: 374.0.

### Synthesis of rac-7-bromo-2-((1S*,2S*)-2-(4-methylpyrimidin-2-yl)cyclopropyl)quinolin-4 (1H)-one

A mixture of rac-(1S*,2S*)—N-(2-acetyl-5-bromophenyl)-2-(4-methylpyrimidin-2-yl)cyclopropane-1-carboxamide (0.78 g, 2.1 mmol) and NaOH (0.33 g, 8.4 mmol) in 1,4-dioxane (15 mL) was stirred at 110° C. for 1 h. The reaction mixture was poured into water (60 mL) then extracted with EtOAc (50 mL×3). The combined organic layers were concentrated and the residue purified by column chromatography on silica gel (eluting with 0˜5% MeOH/DCM) to give rac-7-bromo-2-((1S*,2S*)-2-(4-methylpyrimidin-2-yl)cyclopropyl)quinolin-4 (1H)-one (0.57 g, 76%) as a pale solid.

ESI-MS [M+H]⁺: 356.1.

### Synthesis of rac-7-bromo-4-methoxy-2-((1S*,2S*)-2-(4-methylpyrimidin-2-yl)cyclopropyl)quinoline

A mixture of rac-7-bromo-2-((1S*,2S*)-2-(4-methylpyrimidin-2-yl)cyclopropyl)quinolin-4 (1H)-one (300 mg, 0.84 mmol) and K₂CO₃ (116 mg, 0.84 mmol) in acetone (10 mL) was stirred at reflux for 2 h. After cooling to room temperature, dimethyl sulfate (106 mg, 0.84 mmol) was added and stirred at reflux for another 12 h. The reaction mixture was cooled to room temperature and quenched with H₂O (50 mL). The resulting mixture was concentrated in vacuo to remove acetone and extracted with EtOAc (40 mL×2). The combined organic layers were washed with brine (80 mL×1), dried over Na₂SO₄, and then concentrated in vacuo to get a residue, which was purified by column chromatography on silica gel, eluting with 10˜30% EtOAc/PE to give rac-7-bromo-4-methoxy-2-((1S*,2S*)-2-(4-methylpyrimidin-2-yl)cyclopropyl)quinoline (130 mg, 42%) as a white solid. ESI-MS [M+H]⁺: 370.0.

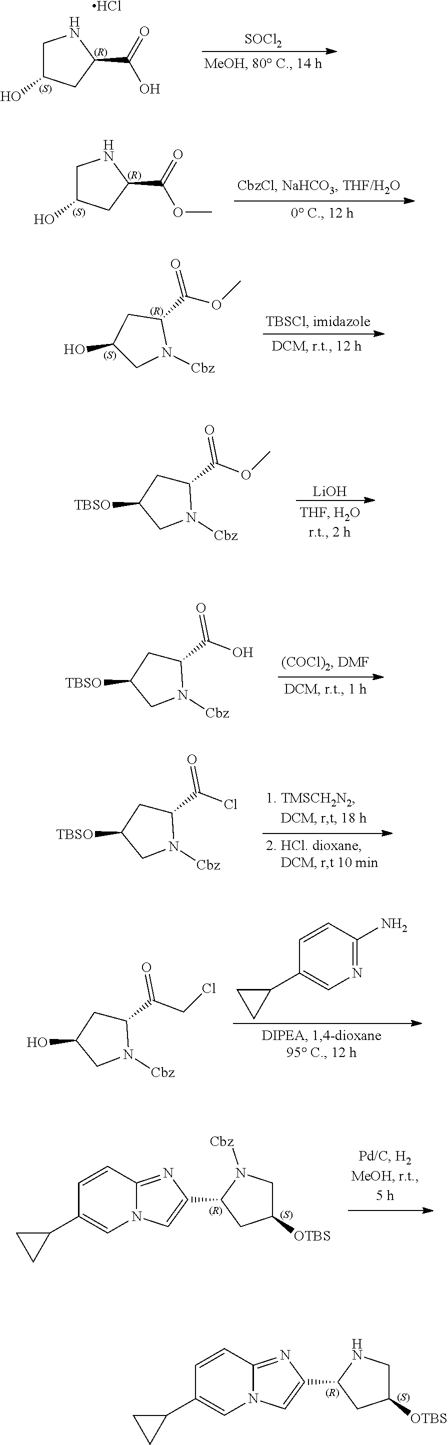

### Synthesis of methyl (2R,4S)-4-hydroxypyrrolidine-2-carboxylate

To a solution of (2R, 4S)-4-hydroxypyrrolidine-2-carboxylic acid hydrochloride (5 g, 29.8 mmol) in MeOH (50 mL) was added SOCl₂ (10.6 g, 89.4 mmol) slowly at 0° C. After stirring at 80° C. for 12 h, the reaction was concentrated in vacuo to give methyl (2R, 4S)-4-hydroxypyrrolidine-2-carboxylate (4 g, crude) as a grey solid. ESI-MS [M+H]+: 146.2.

### Synthesis of 1-benzyl 2-methyl (2R,4S)-4-hydroxypyrrolidine-1,2-dicarboxylate

To a solution of methyl (2R, 4S)-4-hydroxypyrrolidine-2-carboxylate (4 g, 27.5 mmol) in THF (80 mL) was added saturated aqueous NaHCO₃ (60 ml), followed by CbzCl (5.6 g, 33.1 mmol) at 0° C. The reaction mixture was stirred at 0° C. for another 1 h and then extracted with EtOAc (3×50 mL). The combined organic layers were washed with brine, dried over Na₂SO₄ and concentrated in vacuo to give the crude, which was purified by silica gel chromatography (EtOAc/PE=1/5) to give 1-benzyl 2-methyl (2R, 4s)-4-hydroxypyrrolidine-1,2-dicarboxylate (5.8 g, 75%) as yellow oil. ESI-MS [M+H]⁺: 280.1.

### Synthesis of 1-benzyl 2-methyl (2R,4S)-4-((tert-butyldimethylsilyl)oxy)pyrrolidine-1,2-dicarboxylate

A mixture of 1-benzyl 2-methyl (2R,4S)-4-hydroxypyrrolidine-1,2-dicarboxylate (4 g, 14.3 mmol), TMSCl (2.3 g, 21.5 mmol), imidazole (1.9 g, 28.6 mmol) in DCM (40 mL) was stirred at room temperature for 12 h. The reaction was washed with water (50 mL) then extracted with EtOAc (3×50 mL). The combined organic layers were washed with brine, dried over Na₂SO₄, filtered and concentrated in vacuo to give the crude, which was purified by silica gel chromatography (EtOAc/PE=1/3) to give 1-benzyl 2-methyl (2R, 4S)-4-((tert-butyldimethylsilyl)oxy)pyrrolidine-1,2-dicarboxylate (4 g, 71%) as yellow oil. ESI-MS [M+H]⁺: 394.1.

### Synthesis of (2R,4S)-1-((benzyloxy)carbonyl)-4-((tert-butyldimethylsilyl)oxy)pyrrolidine-2-carboxylic acid

To a solution of 1-benzyl 2-methyl (2R,4S)-4-((tert-butyldimethylsilyl)oxy)pyrrolidine-1,2-dicarboxylate (4 g, 10.2 mmol) in THF (40 mL) was added LiOH (1.25 g, 30.6 mmol) and water (4 mL). The reaction mixture was stirred at room temperature for 2 h and then concentrated in vacuo to remove THF. The pH of the resulting residue was adjusted to 3-4 with HCl (1.0 M) then the mixture was extracted with EtOAc (3×50 mL). The combined organic layers were washed with brine, dried over Na₂SO₄, filtered and concentrated in vacuo to give the crude (2R,4S)-1-((benzyloxy)carbonyl)-4-((tert-butyldimethylsilyl)oxy)pyrrolidine-2-carboxylic acid (3.3 g, 87%) as yellow oil. ESI-MS [M+H]⁺: 394.1.

### Synthesis of benzyl (2R,4S)-4-((tert-butyldimethylsilyl)oxy)-2-(chlorocarbonyl)pyrrolidine-1-carboxylate

To a solution of (2R,4S)-1-((benzyloxy)carbonyl)-4-((tert-butyldimethylsilyl)oxy)pyrrolidine-2-carboxylic acid (2 g, crude from previous step) in DCM (20 mL) was added DMF (2 drops) and (COCl)₂ (1.0 g, 7.91 mmol) at 0° C. After stirring at room temperature for 1 h, the solution was concentrated to give benzyl (2R,4S)-4-((tert-butyldimethylsilyl)oxy)-2-(chlorocarbonyl)pyrrolidine-1-carboxylate (2.1 g crude) as yellow oil, which was used in next step without further purification. ESI-MS [M+H]⁺: 394.1.

### Synthesis of benzyl (2R,4S)-2-(2-chloroacetyl)-4-hydroxypyrrolidine-1-carboxylate

To a solution of benzyl (2R, 4S)-4-((tert-butyldimethylsilyl)oxy)-2-(chlorocarbonyl)pyrrolidine-1-carboxylate (400 mg, 1 mmol) in DCM (20 mL) was added TMSCH₂N₂ (1 mL, 2 M in hexane). After stirring at room temperature for 12 h, the solution was concentrated in vacuo to give the crude, which was re-dissolved in DCM (10 mL) and then HCl (4.0 M in 1,4-dioxane, 0.5 mL) was added. After stirring at room temperature for another 10 min, the solution was concentrated to give the crude, which was purified by silica gel chromatography (eluent: EtOAc/PE=1/5) to give benzyl (2R,4S)-2-(2-chloroacetyl)-4-hydroxypyrrolidine-1-carboxylate (150 mg, 36%) as yellow oil. ESI-MS [M+H]⁺: 298.2.

### Synthesis of benzyl (2R, 4S)-4-((tert-butyldimethylsilyl)oxy)-2-(6-cyclopropylimidazo[1,2-a]pyridin-2-yl)pyrrolidine-1-carboxylate

A solution of benzyl (2R,4S)-2-(2-chloroacetyl)-4-hydroxypyrrolidine-1-carboxylate (150 mg, 0.51 mmol), 5-cyclopropylpyridin-2-amine (134 mg, 1.0 mmol) and DIPEA (329 mg, 2.55 mmol) in 1.4-dioxane (10 mL) was stirred at 95° C. for 12 h. The resulting mixture was concentrated in vacuo to give the crude, which was purified with silica gel chromatography (eluent: DCM/MeOH=15/1) to give benzyl (2R,4S)-4-((tert-butyldimethylsilyl)oxy)-2-(6-cyclopropylimidazo[1,2-a]pyridin-2-yl)pyrrolidine-1-carboxylate (150 mg, 58%) as a yellow solid. ESI-MS [M+H]⁺: 492.1.

### Synthesis of 2-((2R,4S)-4-((tert-butyldimethylsilyl)oxy)pyrrolidin-2-yl)-6-cyclopropylimidazo[1,2-a]pyridine

A mixture of benzyl (2R,4S)-4-((tert-butyldimethylsilyl)oxy)-2-(6-cyclopropylimidazo[1,2-a]pyridin-2-yl)pyrrolidine-1-carboxylate (150 mg, 0.31 mmol) and Pd/C (20 mg) in MeOH (10 mL) was stirred at room temperature for 5 h under a H₂ atmosphere. The reaction was filtered, washed with MeOH (30 mL). The filtrate was concentrated in vacuo to give 2-((2R,4S)-4-((tert-butyldimethylsilyl)oxy)pyrrolidin-2-yl)-6-cyclopropylimidazo[1,2-a]pyridine (120 mg, crude) as a yellow solid. ESI-MS [M+H]⁺: 358.2.

### Synthesis of 7-((2R,4S)-4-((tert-butyldimethylsilyl)oxy)-2-(6-cyclopropylimidazo[1,2-a]pyridin-2-yl)pyrrolidin-1-yl)-4-methoxy-2-((1S*,2S*)-2-(4-methylpyrimidin-2-yl)cyclopropyl)quinoline

A mixture of rac-7-bromo-4-methoxy-2-((1S*,2S*)-2-(4-methylpyrimidin-2-yl)cyclopropyl)quinoline (130 mg, 0.35 mmol), 2-((2R,4S)-4-((tert-butyldimethylsilyl)oxy)pyrrolidin-2-yl)-6-cyclopropylimidazo[1,2-a]pyridine (125 mg, 0.35 mmol), Pd₂(dba)₃ (16 mg, 0.017 mmol), BINAP (21 mg, 0.034 mmol) and t-BuOK (76 mg, 0.68 mmol) in toluene (10 mL) was stirred at 90° C. for 6 h under N₂. After the reaction was cooled to room temperature, water (50 mL) was added and extracted with EtOAc (50 mL×3). The combined organic layers were washed with brine (50 mL), dried over Na₂SO₄, and then concentrated in vacuo to get a residue, which was purified by Prep-HPLC to give the desired 7-((2R,4S)-4-((tert-butyldimethylsilyl)oxy)-2-(6-cyclopropylimidazo[1,2-a]pyridin-2-yl)pyrrolidin-1-yl)-4-methoxy-2-((1S*,2S*)-2-(4-methylpyrimidin-2-yl)cyclopropyl)quinoline (30 mg, 13%) as a yellow solid. Also isolated was the undesired 7-(2-((2R,4S)-4-((tert-butyldimethylsilyl)oxy)pyrrolidin-2-yl)-6-cyclopropylimidazo[1,2-a]pyridin-3-yl)-4-methoxy-2-((1S*,2S*)-2-(4-methylpyrimidin-2-yl)cyclopropyl)quinoline (40 mg, 18%) as a yellow solid.

ESI-MS [M+H]⁺: 647.4.

### Example 53

### Synthesis of (3S,5R)-5-(6-cyclopropylimidazo[1,2-a]pyridin-2-yl)-1-(4-methoxy-2-(rac-(1S*,2S*)-2-(4-methylpyrimidin-2-yl)cyclopropyl)quinolin-7-yl)pyrrolidin-3-ol (I-53)

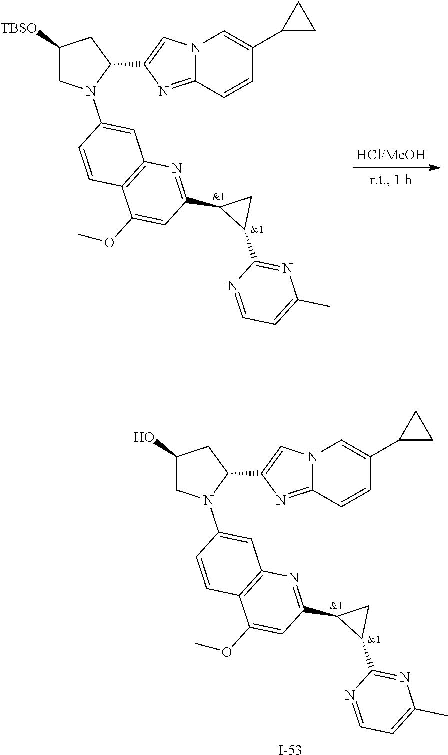

A solution of 7-((2R,4S)-4-((tert-butyldimethylsilyl)oxy)-2-(6-cyclopropylimidazo[1,2-a]pyridin-2-yl)pyrrolidin-1-yl)-4-methoxy-2-((1S*,2S*)-2-(4-methylpyrimidin-2-yl)cyclopropyl) quinoline (30 mg, 0.044 mmol) in HCl (4 N HCl in dioxane, 2.0 mL) and MeOH (2.0 mL) was stirred at room temperature for 1 h. Water (30 mL) was added, pH of the reaction mixture was adjusted to 8˜9 by adding NaHCO₃ (sat. aq.) and then extracted with EtOAc (30 mL×3). The combined organic layers were concentrated in vacuo to get a residue, which was purified by column chromatography on silica gel, eluting with 0˜12% MeOH/DCM to give (3S,5R)-5-(6-cyclopropylimidazo[1,2-a]pyridin-2-yl)-1-(4-methoxy-2-(rac-(1S*,2S*)-2-(4-methylpyrimidin-2-yl)cyclopropyl)quinolin-7-yl)pyrrolidin-3-ol (15 mg, 64%) as a yellow solid.

ESI-MS [M+H]⁺: 533.3, ¹H NMR (400 MHz, DMSO) δ 8.54 (d, J=5.1 Hz, 1H), 8.22 (d, J=6.6 Hz, 1H), 7.85-7.75 (m, 1H), 7.58 (d, J=4.1 Hz, 1H), 7.39 (dd, J=9.3, 4.0 Hz, 1H), 7.20 (d, J=4.6 Hz, 1H), 7.07-6.90 (m, 2H), 6.80-6.65 (m, 2H), 5.26-5.10 (m, 2H), 4.62-4.52 (m, 1H), 4.01 (s, 3H), 3.96-3.87 (m, 1H), 3.33-3.30 (m, 1H), 2.95-2.65 (m, 2H), 2.42 (s, 3H), 2.37-2.32 (m, 1H), 2.30-2.23 (m, 1H), 1.93-1.83 (m, 2H), 1.81-1.72 (m, 1H), 0.92-0.84 (m, 2H), 0.68-0.58 (m, 2H).

### Example 54

### Synthesis of rac-7-((2R,4S)-2-(6-cyclopropylimidazo[1,2-a]pyridin-2-yl)-4-hydroxypyrrolidin-1-yl)-2-((1S*,2S*)-2-(4-methylpyrimidin-2-yl)cyclopropyl)quinolin-4-01 (I-54)

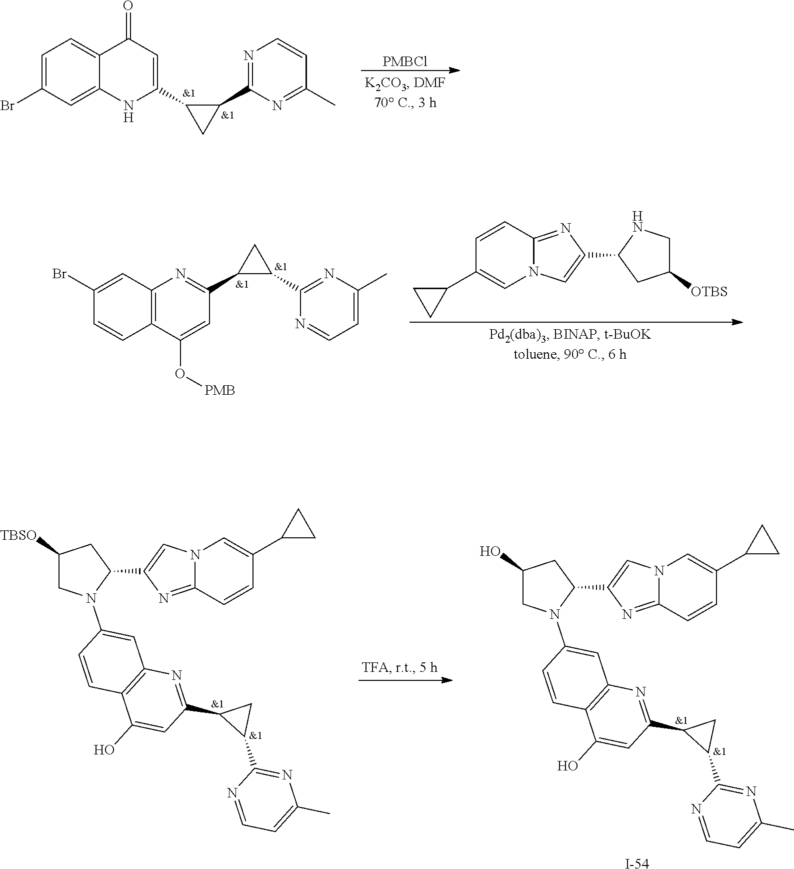

### Synthesis of rac-7-bromo-4-((4-methoxybenzyl)oxy)-2-((1S*,2S*)-2-(4-methylpyrimidin-2-yl)cyclopropyl)quinoline

A mixture of rac-7-bromo-2-((1S*,2S*)-2-(4-methylpyrimidin-2-yl)cyclopropyl)quinolin-4 (1H)-one (192 mg, 0.54 mmol), 1-(chloromethyl)-4-methoxybenzene (109.2 mg, 0.7 mmol) and K₂CO₃ (124.2 mg, 0.90 mmol) in DMF (6.0 mL) was stirred at 70° C. for 3 h. The reaction mixture was poured into water (40 mL) and then extracted with EtOAc (40 mL×3). The combined organic layers were washed with brine (40 mL×1), dried over Na₂SO₄, and then concentrated in vacuo to get a residue, which was purified by column chromatography on silica gel, eluting with 10˜30% EtOAc/PE to give rac-7-bromo-4-((4-methoxybenzyl)oxy)-2-((1S*,2S*)-2-(4-methylpyrimidin-2-yl)cyclopropyl)quinoline (190 mg, 74%) as a yellow solid. ESI-MS [M+H]⁺: 476.0

### Synthesis of 7-((2R,4S)-4-((tert-butyldimethylsilyl)oxy)-2-(6-cyclopropylimidazo[1,2-a]pyridin-2-yl)pyrrolidin-1-yl)-4-((4-methoxybenzyl)oxy)-2-((1S*,2S*)-2-(4-methylpyrimidin-2-yl)cyclopropyl)quinoline

A mixture of rac-7-bromo-4-((4-methoxybenzyl)oxy)-2-((1S*,2S*)-2-(4-methylpyrimidin-2-yl)cyclopropyl)quinoline (70 mg, 0.15 mmol), 2-((2R,4S)-4-((tert-butyldimethylsilyl)oxy)pyrrolidin-2-yl)-6-cyclopropylimidazo[1,2-a]pyridine (54 mg, 0.15 mmol), Pd₂(dba)₃ (6.9 mg, 0.0075 mmol), BINAP (9.3 mg, 0.015 mmol) and t-BuOK (34 mg, 0.30 mmol) in toluene (4.0 mL) was stirred at 90° C. for 6 h under N₂. The reaction mixture was diluted in water (30 mL) and then extracted with EtOAc (30 mL×2). The combined organic layers were washed with brine (30 mL), dried over Na₂SO₄, and then concentrated to get a residue, which was purified by column chromatography on silica gel, eluting with 0˜60% EtOAc/PE to give 7-((2R,4S)-4-((tert-butyldimethylsilyl)oxy)-2-(6-cyclopropylimidazo[1,2-a]pyridin-2-yl)pyrrolidin-1-yl)-4-((4-methoxybenzyl)oxy)-2-((1S*,2S*)-2-(4-methylpyrimidin-2-yl)cyclopropyl)quinoline (74 mg, 65%) as a yellow solid. ESI-MS [M+H]⁺: 753.3.

### Synthesis of rac-7-((2R,4S)-2-(6-cyclopropylimidazo[1,2-a]pyridin-2-yl)-4-hydroxypyrrolidin-1-yl)-2-((1S*,2S*)-2-(4-methylpyrimidin-2-yl)cyclopropyl)quinolin-4-ol

A mixture of 7-((2R,4S)-4-((tert-butyldimethylsilyl)oxy)-2-(6-cyclopropylimidazo[1,2-a]pyridin-2-yl)pyrrolidin-1-yl)-4-((4-methoxybenzyl)oxy)-2-((1S*,2S*)-2-(4-methylpyrimidin-2-yl)cyclopropyl) quinoline (74 mg, 0.098 mmol) in TFA (4.0 mL) was stirred at room temperature for 5 h. The reaction mixture was concentrated in vacuo and the residue was diluted with NaHCO₃ (sat. aq., 20 mL) then extracted with EtOAc (30 mL×3). The combined organic layers were washed with brine (30 mL), dried over Na₂SO₄, concentrated in vacuo to get a residue, which was purified by column chromatography on silica gel, eluting with 0˜10% MeOH/DCM to give 7-((2R,4S)-2-(6-cyclopropylimidazo[1,2-a]pyridin-2-yl)-4-hydroxypyrrolidin-1-yl)-2-(rac-(1S*,2S*)-2-(4-methylpyrimidin-2-yl)cyclopropyl)quinolin-4-ol (35 mg, 69%) as a pale solid.

ESI-MS [M+H]⁺: 519.2, ¹H NMR (400 MHz, DMSO) δ 11.04 (s, 1H), 8.54-8.52 (m, 1H), 8.20 (d, J=6.1 Hz, 1H), 7.72 (d, J=9.1 Hz, 1H), 7.52-7.44 (m, 1H), 7.41-7.33 (m, 1H), 7.23-7.17 (m, 1H), 7.03-6.91 (m, 1H), 6.70-6.58 (m, 1H), 6.37 (s, 1H), 5.59 (s, 1H), 5.17 (d, J=4.5 Hz, 1H), 5.08-4.96 (m, 1H), 4.58-4.45 (m, 1H), 3.88-3.80 (m, 1H), 3.29-3.21 (m, 1H), 2.59-2.53 (m, 2H), 2.42 (d, J=3.5 Hz, 3H), 2.31-2.22 (m, 2H), 1.92-1.83 (m, 1H), 1.70-1.60 (m, 2H), 0.91-0.83 (m, 2H), 0.66-0.58 (m, 2H).

### Example 55

### Synthesis of rac-6-(((6-cyclopropylimidazo[1,2-a]pyridin-2 yl)methyl)amino)-3-((1S*,2S*)-2-(4-methylpyrimidin-2-yl)cyclopropyl)-4H-benzo[b][1,4]thiazine 1,1-dioxide (I-55)

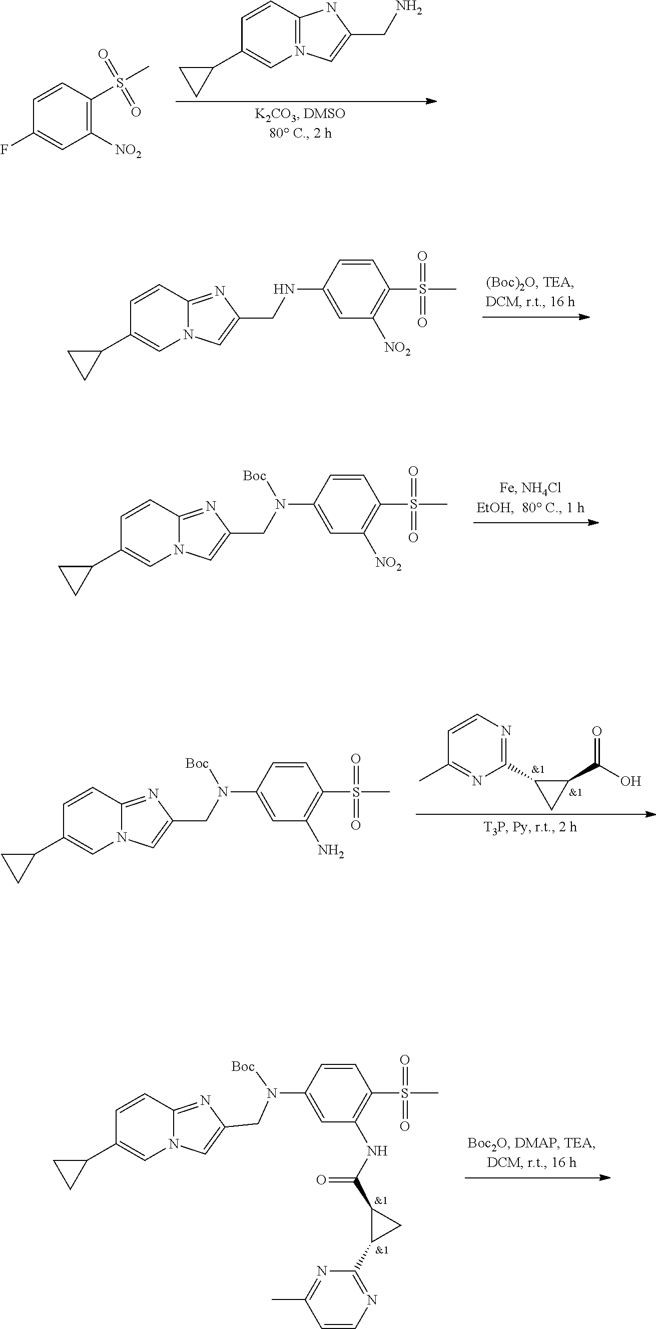

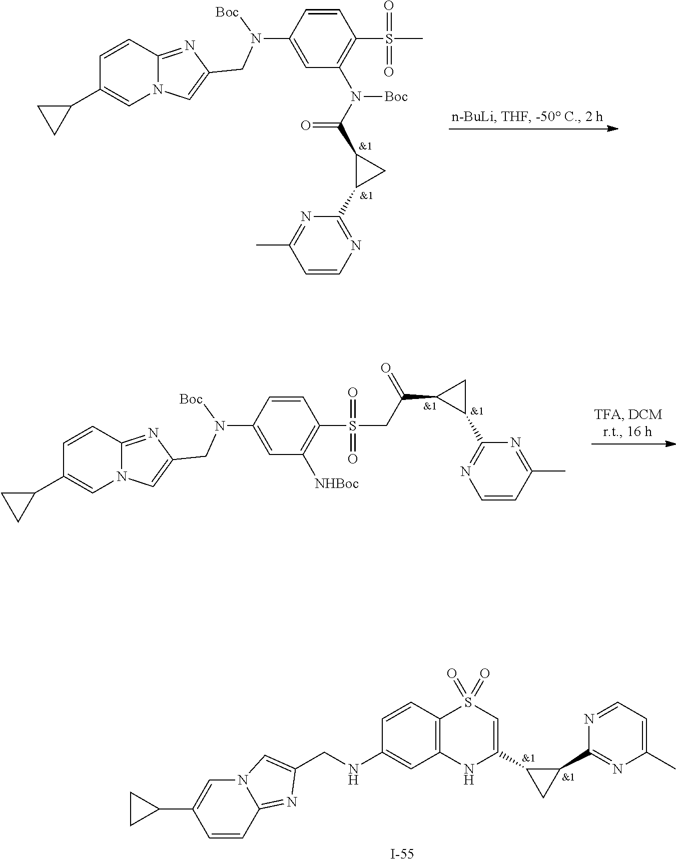

### Synthesis of N-((6-cyclopropylimidazo[1,2-a]pyridin-2-yl)methyl)-4-(methylsulfonyl)-3-nitroaniline

A mixture of 4-fluoro-1-(methylsulfonyl)-2-nitrobenzene (430 mg, 1.96 mmol), (6-cyclopropylimidazo[1,2-a]pyridin-2-yl)methanamine (403 mg, 2.16 mmol) and K₂CO₃ (676 mg, 4.9 mmol) in DMSO (10 mL) was stirred at 80° C. for 2 h under N₂. The reaction was cooled to room temperature, diluted with water (50 mL) and extracted with EtOAc (40 mL×3). The combined organic layers were washed with brine (40 mL), dried over anhydrous Na₂SO₄, and concentrated in vacuo to give N-((6-cyclopropylimidazo[1,2-a]pyridin-2-yl)methyl)-4-(methylsulfonyl)-3-nitroaniline (740 mg, crude) as a yellow oil, which was used into next step without further purification. ESI-MS [M+H]+: 387.1.

### Synthesis of tert-butyl ((6-cyclopropylimidazo[1,2-a]pyridin-2-yl)methyl)(4-(methylsulfonyl)-3-nitrophenyl)carbamate

A mixture of N-((6-cyclopropylimidazo[1,2-a]pyridin-2-yl)methyl)-4-(methylsulfonyl)-3-nitroaniline (740 mg crude), di-tert-butyl dicarbonate (820 mg, 3.8 mmol), DMAP (23 mg, 0.19 mmol) and TEA (576 mg, 5.7 mmol) in DCM (30 mL) was stirred at room temperature for 16 h under N₂. The reaction was concentrated to give the crude product, which was purified by silica gel column chromatography eluting with a gradient of 1-5% MeOH in DCM to give tert-butyl ((6-cyclopropylimidazo[1,2-a]pyridin-2-yl)methyl)(4-(methylsulfonyl)-3-nitrophenyl)carbamate (900 mg, 95% over 2 steps) as a yellow solid. ESI-MS [M+H]⁺: 487.2.

### Synthesis of tert-butyl (3-amino-4-(methylsulfonyl)phenyl)((6-cyclopropylimidazo[1,2-a]pyridin-2-yl)methyl)carbamate

A mixture of tert-butyl ((6-cyclopropylimidazo[1,2-a]pyridin-2-yl)methyl)(4-(methylsulfonyl)-3-nitrophenyl)carbamate (900 mg, 1.85 mmol), NH₄Cl (1.08 g, 20 mmol) and Fe powder (311 mg, 5.55 mmol) in EtOH (20 mL) was stirred at 80° C. for 1 h under N₂, The reaction mixture was cooled to room temperature and filtered through Celite®, the filter cake was washed with DCM/MeOH (10/1, 50 mL). The filtrate was concentrated in vacuo to give the crude, which was purified by silica gel column chromatography eluting with a gradient of 0-4% MeOH in DCM to give tert-butyl (3-amino-4-(methylsulfonyl)phenyl)((6-cyclopropylimidazo[1,2-a]pyridin-2-yl)methyl)carbamate (450 mg, 53%) as a yellow solid. ESI-MS [M+H]⁺: 457.2.

### Synthesis of rac-tert-butyl ((6-cyclopropylimidazo[1,2-a]pyridin-2-yl)methyl)(3-((1S*,2S*)-2-(4-methylpyrimidin-2-yl)cyclopropane-1-carboxamido)-4-(methylsulfonyl)phenyl)carbamate

To a mixture of tert-butyl (3-amino-4-(methylsulfonyl)phenyl)((6-cyclopropylimidazo[1,2-a]pyridin-2-yl)methyl)carbamate (200 mg, 0.44 mmol), rac-(1S*,2S*)-2-(4-methylpyrimidin-2-yl)cyclopropane-1-carboxylic acid (78 mg, 0.44 mmol) in pyridine (5 mL) was added T3P (4.2 g, 6.6 mmol, 50% solution in EtOAc) dropwise at room temperature under N₂. The mixture was stirred at room temperature for 2 h. Water (30 mL) was added and the mixture was extracted with EtOAc (30 mL×3). The combined organic layers were washed with brine (30 mL), dried over anhydrous Na₂SO₄, and concentrated in vacuo to give the crude product, which was purified by preparative TLC (eluent: DCM/MeOH=15/1) to give rac-tert-butyl ((6-cyclopropylimidazo[1,2-a]pyridin-2-yl)methyl)(3-((1S*,2S*)-2-(4-methylpyrimidin-2-yl)cyclopropane-1-carboxamido)-4-(methylsulfonyl)phenyl) carbamate (200 mg, 74%) as a yellow solid. ESI-MS [M+H]+: 617.3.

### Synthesis of rac-tert-butyl (3-((1S*,2S*)—N-(tert-butoxycarbonyl)-2-(4-methylpyrimidin-2-yl)cyclopropane-1-carboxamido)-4-(methylsulfonyl)phenyl)((6-cyclopropylimidazo[1,2-a]pyridin-2-yl)methyl)carbamate

To a mixture of rac-tert-butyl ((6-cyclopropylimidazo[1,2-a]pyridin-2-yl)methyl)(3-((1S*,2S*)-2-(4-methylpyrimidin-2-yl)cyclopropane-1-carboxamido)-4-(methylsulfonyl)phenyl) carbamate (100 mg, 0.16 mmol) in DCM (5 mL) was added di-tert-butyl dicarbonate (70 mg, 0.32 mmol), DMAP (2 mg, 0.016 mmol) and TEA (48 mg, 0.48 mmol). The mixture was stirred at room temperature for 16 h. The mixture was diluted with DCM (50 mL), washed with H₂O (20 mL×2), brine (20 mL), dried over anhydrous Na₂SO₄, and concentrated in vacuo to give the crude product, which was purified by preparative TLC (eluent: DCM/MeOH=15/1) to give rac-tert-butyl (3-((1S*,2S*)—N-(tert-butoxycarbonyl)-2-(4-methylpyrimidin-2-yl)cyclopropane-1-carboxamido)-4-(methylsulfonyl)phenyl)((6-cyclopropylimidazo[1,2-a]pyridin-2-yl)methyl)carbamate (100 mg, 87%) as a yellow solid. ESI-MS [M+H]⁺: 717.2

### Synthesis of rac-tert-butyl (3-((tert-butoxycarbonyl)amino)-4-((2-((1S*,2S*)-2-(4-methylpyrimidin-2-yl)cyclopropyl)-2-oxoethyl)sulfonyl)phenyl)((6-cyclopropylimidazo[1,2-a]pyridin-2-yl)methyl)carbamate

To a solution of rac-tert-butyl (3-((1S*,2S*)—N-(tert-butoxycarbonyl)-2-(4-methylpyrimidin-2-yl)cyclopropane-1-carboxamido)-4-(methylsulfonyl)phenyl)((6-cyclopropylimidazo[1,2-a]pyridin-2-yl)methyl)carbamate (100 mg, 0.14 mmol) in THF (10 mL) was added n-BuLi (2.4 M, 0.26 mL, 0.63 mmol) dropwise at −50° C. under N₂. The mixture was stirred at −50° C. for 2 h under N₂. The reaction was quenched with NH₄Cl (sat. aq., 20 mL) and extracted with EtOAc (30 mL×3). The combined organic layers were washed with brine (30 mL), dried over anhydrous Na₂SO₄, and concentrated in vacuo to give the crude, which was purified by preparative TLC (eluent: DCM/MeOH=15/1) to give rac-tert-butyl (3-((tert-butoxycarbonyl)amino)-4-((2-((1S*,2S*)-2-(4-methylpyrimidin-2-yl)cyclo propyl)-2-oxoethyl)sulfonyl)phenyl)((6-cyclopropylimidazo[1,2-a]pyridin-2-yl)methyl)carbamate (80 mg, 80%) as a white solid. ESI-MS [M+H]⁺: 717.3

### Synthesis of rac-6-(((6-cyclopropylimidazo[1,2-a]pyridin-2-yl)methyl)amino)-3-((1S*,2S*)-2-(4-methylpyrimidin-2-yl)cyclopropyl)-4H-benzo[b][1,4]thiazine 1,1-dioxide

To a solution of rac-tert-butyl (3-((tert-butoxycarbonyl)amino)-4-((2-((1S*,2S*)-2-(4-methylpyrimidin-2-yl)cyclopropyl)-2-oxoethyl)sulfonyl)phenyl)((6-cyclopropylimidazo[1,2-a]pyridin-2-yl)methyl)carbamate (80 mg, 0.11 mmol) in DCM (2 mL) was added TFA (1 mL) and the mixture was stirred at room temperature for 16 h. The mixture was concentrated in vacuo. The residue was washed with NaHCO₃ (sat. aq., 20 mL) and extracted with DCM (20 mL×3). The combined organic layers were washed with brine (30 mL), dried over anhydrous Na₂SO₄, concentrated in vacuo to give the crude, which was purified by preparative TLC (eluent: DCM/MeOH=10/1) to give rac-6-(((6-cyclopropylimidazo[1,2-a]pyridin-2-yl)methyl)amino)-3-((1S*,2S*)-2-(4-methylpyrimidin-2-yl)cyclopropyl)-4H-benzo[b][1,4]thiazine 1,1-dioxide (20 mg, 36%) as a white solid.

ESI-MS [M+H]⁺: 499.2, ¹H NMR (400 MHz, DMSO) δ 10.24 (s, 1H), 8.53 (d, J=5.1 Hz, 1H), 8.29 (s, 1H), 7.59 (s, 1H), 7.43-7.36 (m, 2H), 7.20 (d, J=5.1 Hz, 1H), 7.03 (t, J=5.8 Hz, 1H), 6.96 (dd, J=9.3, 1.6 Hz, 1H), 6.61 (dd, J=8.9, 2.0 Hz, 1H), 6.27 (d, J=1.9 Hz, 1H), 5.70 (s, 1H), 4.36 (d, J=5.7 Hz, 2H), 2.47-2.44 (m, 1H), 2.41 (s, 3H), 2.32-2.27 (m, 1H), 1.93-1.86 (m, 1H), 1.64-1.56 (m, 1H), 1.56-1.52 (m, 1H), 0.92-0.87 (m, 2H), 0.66-0.64 (m, 2H).

### Example 56

### Synthesis of rac-(3S,5R)-1-(2-((1S*,2S*)-2-(3-chlorophenyl)cyclopropyl)-3-fluoro-1H-pyrrolo[3,2-c]pyridin-6-yl)-5-(6-cyclopropylimidazo[1,2-a]pyridin-2-yl)pyrrolidin-3-ol (I-56)

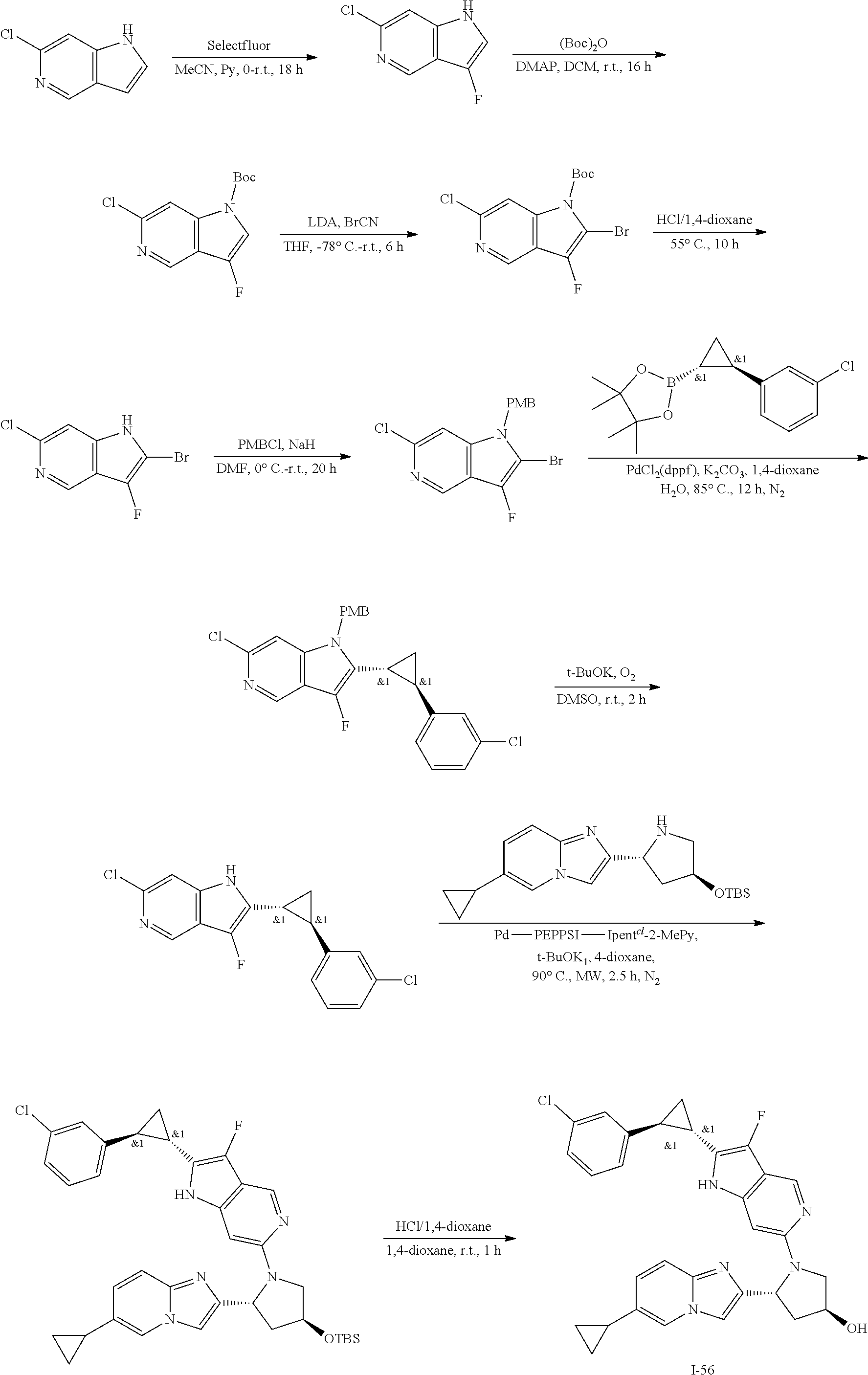

### Synthesis of 6-chloro-3-fluoro-1H-pyrrolo[3,2-c]pyridine

To a mixture of 6-chloro-1H-pyrrolo[3,2-c]pyridine (5.0 g, 33 mmol) in MeCN (50 mL) was added pyridine (15 mL), followed by Selectfluor® (14 g, 39 mmol) at 0° C. and the reaction was stirred at room temperature for 18 h. The reaction was concentrated in vacuo. The residue was diluted with water (50 mL), and then extracted with EtOAc (70 mL×3). The combined organic layers were washed with brine (40 mL), dried over Na₂SO₄, filtered, and concentrated in vacuo. The residue was purified by column chromatography (eluent: MeOH/DCM=0-10%) to afford 6-chloro-3-fluoro-1H-pyrrolo[3,2-c]pyridine (3.1 g, 55%) as a red solid.

ESI-MS [M+H]+: 171.2

### Synthesis of tert-butyl 6-chloro-3-fluoro-1H-pyrrolo[3,2-c]pyridine-1-carboxylate

To a mixture of 6-chloro-3-fluoro-1H-pyrrolo[3,2-c]pyridine (3.1 g, 18 mmol) and DMAP (220 mg, 1.8 mmol) in DCM (45 mL) was added (Boc)₂O (4.4 g, 20 mmol). The reaction mixture was stirred at room temperature for 16 h. The reaction mixture was diluted with DCM (50 mL), and then washed with water (50 mL). The organic phase was washed with brine (50 mL), dried over Na₂SO₄, filtered and concentrated in vacuo to give the crude, which was purified with column chromatography on silica gel (eluent: EtOAc/PE=0-20%) to afford tert-butyl 6-chloro-3-fluoro-1H-pyrrolo[3,2-c]pyridine-1-carboxylate (3 g, 61%) as a white solid.

ESI-MS [M+H]+: 271.2

### Synthesis of tert-butyl 2-bromo-6-chloro-3-fluoro-1H-pyrrolo[3,2-c]pyridine-1-carboxylate

To a solution of tert-butyl 6-chloro-3-fluoro-1H-pyrrolo[3,2-c]pyridine-1-carboxylate (3 g, 11 mmol) in THF (90 mL) was added LDA (6 mL, 12 mmol, 2M in THF) slowly at −78° C. After stirring for 30 min at −78° C., a solution of BrCN (4.2 g, 39 mmol) in THF (10 mL) was added at −78° C. The reaction was warmed to room temperature and stirred for 6 h under N₂. The reaction was quenched with NH₄Cl (Sat. aq., 50 mL) and then extracted with EtOAc (40 mL×3). The combined organic layers were washed with brine (60 mL), dried over Na₂SO₄, filtered, and concentrated in vacuo. The residue was purified by column chromatography on silica gel chromatography (eluent: EtOAc/PE=0-10%) to afford tert-butyl 2-bromo-6-chloro-3-fluoro-1H-pyrrolo[3,2-c]pyridine-1-carboxylate (3.4 g, yield 88%) as a white solid.

ESI-MS [M+H]+: 349.2

### Synthesis of 2-bromo-6-chloro-3-fluoro-1H-pyrrolo[3,2-c]pyridine

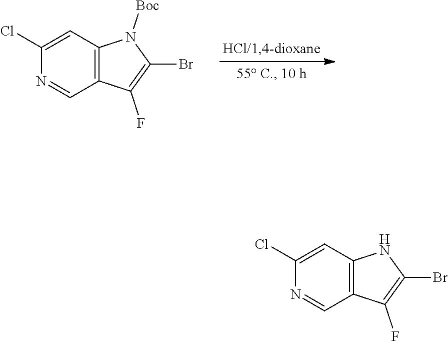 [SMILES: CC(C)(C)OC(=O)n1c(Br)c(F)c2cnc(Cl)cc21 ; Fc1c(Br)[nH]c2cc(Cl)ncc12]

tert-butyl 2-bromo-6-chloro-3-fluoro-1H-pyrrolo[3,2-c]pyridine-1-carboxylate (3.4 g, 9.8 mmol) in HCl/1,4-dioxane (60 mL, 4 M in 1,4-dioxane) was stirred at 55° C. for 10 h. The reaction was concentrated in vacuo to afford 2-bromo-6-chloro-3-fluoro-1H-pyrrolo[3,2-c]pyridine (2.9 g, quant) as a white solid, which was used in next step without purification.

ESI-MS [M+H]+: 249.2

### Synthesis of 2-bromo-6-chloro-3-fluoro-1-(4-methoxybenzyl)-1H-pyrrolo[3,2-c]pyridine

To a solution of 2-bromo-6-chloro-3-fluoro-1H-pyrrolo[3,2-c]pyridine (1.1 g, 4.4 mmol) in DMF (30 mL) was added NaH (0.44 g, 11 mmol, 60% dispersion in mineral oil) at 0° C. under N₂. After stirred for 1 h, a solution of PMBCl (1.4 g, 8.8 mmol) in DMF (2 mL) was added. The resulting reaction was stirred at room temperature for 20 h under N₂. The reaction was quenched with NH₄Cl (Sat. aq., 30 mL), and then extracted with EtOAc (30 mL×3). The combined organic phase was dried over Na₂SO₄ and concentrated in vacuo. The residue was purified by column chromatography on silica gel (eluent: EtOAc/PE=0-10%) to afford 2-bromo-6-chloro-3-fluoro-1-(4-methoxybenzyl)-1H-pyrrolo[3,2-c]pyridine (1.2 g, yield 74%) as white solid. ESI-MS [M+H]+: 369.2

### Synthesis of rac-6-chloro-2-((1S*,2S*)-2-(3-chlorophenyl)cyclopropyl)-3-fluoro-1-(4-methoxybenzyl)-1H-pyrrolo[3,2-c]pyridine

To a mixture of 2-bromo-6-chloro-3-fluoro-1-(4-methoxybenzyl)-1H-pyrrolo[3,2-c]pyridine (1.2 g, 3.4 mmol) in 1,4-dioxane (30 mL) and H₂O (3 mL) was added rac-2-((1S*,2S*)-2-(3-chlorophenyl)cyclopropyl)-4,4,5,5-tetramethyl-1,3,2-dioxaborolane (1.2 g, 4.2 mmol), Pd(dppf)Cl₂ (0.25 g, 0.34 mmol) and K₂CO₃ (1.4 g, 10 mmol). The mixture was stirred at 85° C. for 12 h under N₂. The reaction mixture was cooled to room temperature and filtered, the filtrate was diluted with H₂O (80 mL) and extracted with EtOAc (50 mL×3). The combined organic phase was washed with brine (60 mL), dried over anhydrous Na₂SO₄, filtered and concentrated in vacuo to give the crude, which was purified by column chromatography on silica gel (eluent: EtOAc/PE=0-10%) to afford rac-6-chloro-2-((1S*,2S*)-2-(3-chlorophenyl)cyclopropyl)-3-fluoro-1-(4-methoxybenzyl)-1H-pyrrolo[3,2-c]pyridine (1 g, yield 67%) as pale-yellow sticky oil. ESI-MS [M+H]+: 441.2

### Synthesis of rac-6-chloro-2-((1S*,2S*)-2-(3-chlorophenyl)cyclopropyl)-3-fluoro-1H-pyrrolo[3,2-c]pyridine

To a solution of rac-6-chloro-2-((1S*,2S*)-2-(3-chlorophenyl)cyclopropyl)-3-fluoro-1-(4-methoxybenzyl)-1H-pyrrolo[3,2-c]pyridine (0.40 g, 0.9 mmol) in DMSO (10 mL) was added dropwise t-BuOK (7.8 mL, 7.8 mmol, 1M solution in THF). The reaction was stirred at room temperature for 2 h under an atmosphere of 02. The reaction was quenched with NH₄Cl (Sat. aq., 40 mL), and then extracted with EtOAc (30 mL×3). The combined organic phase was washed with brine (30 mL), dried over Na₂SO₄, filtered and concentrated in vacuo. The residue was purified by column chromatography on silica gel (eluent: EtOAc/PE=0-10%) to afford rac-6-chloro-2-((1S*,2S*)-2-(3-chlorophenyl)cyclopropyl)-3-fluoro-1H-pyrrolo[3,2-c]pyridine (100 mg, 35%) as pale-yellow solid.

ESI-MS [M+H]+: 321.2

### Synthesis of 2-((2R,4S)-4-((tert-butyldimethylsilyl)oxy)-1-(2-((1S*,2S*)-2-(3-chlorophenyl)cyclopropyl)-3-fluoro-1H-pyrrolo[3,2-c]pyridin-6-yl)pyrrolidin-2-yl)-6-cyclopropylimidazo[1,2-a]pyridine

A mixture of rac-6-chloro-2-((1S*,2S*)-2-(3-chlorophenyl)cyclopropyl)-3-fluoro-1H-pyrrolo[3,2-c]pyridine (40 mg, 0.12 mmol), 2-((2R,4S)-4-((tert-butyldimethylsilyl)oxy)pyrrolidin-2-yl)-6-cyclopropylimidazo[1,2-a]pyridine (43 mg, 0.12 mmol), Pd-PEPPSI-Ipentcl-2-MePy (20 mg, 0.024 mmol) and t-BuOK (40 mg, 0.36 mmol) in 1,4-dioxane (5 mL) in a sealed tube was irradiated in microwave at 90° C. for 2.5 h. The reaction mixture was filtered through Celite® and the filter cake was washed with DCM/MeOH (10/1, 100 mL). The filtrate was concentrated in vacuo to give the crude, which was purified by Prep-HPLC (Waters MS-triggered Prep-LC with QDA detector X bridge 5u C18 150×19 mm 20 ml/min 0.1% NH₃ in H₂O-ACN) to afford 2-((2R, 4S)-4-((tert-butyldimethylsilyl)oxy)-1-(2-((1S*,2S*)-2-(3-chlorophenyl)cyclopropyl)-3-fluoro-1H-pyrrolo[3,2-c]pyridin-6-yl)pyrrolidin-2-yl)-6-cyclopropylimidazo[1,2-a]pyridine (5.5 mg, yield 7%) as pale-yellow solid.

ESI-MS [M+H]+: 642.2

### Synthesis of rac-(3S,5R)-1-(2-((1S*,2S*)-2-(3-chlorophenyl)cyclopropyl)-3-fluoro-1H-pyrrolo[3,2-c]pyridin-6-yl)-5-(6-cyclopropylimidazo[1,2-a]pyridin-2-yl)pyrrolidin-3-ol

To a solution of 2-((2R,4S)-4-((tert-butyldimethylsilyl)oxy)-1-(2-((1S*,2S*)-2-(3-chlorophenyl)cyclopropyl)-3-fluoro-1H-pyrrolo[3,2-c]pyridin-6-yl)pyrrolidin-2-yl)-6-cyclopropylimidazo[1,2-a]pyridine (5.5 mg, 0.0086 mmol) in 1,4-dioxane (1 mL) was added HCl (0.5 mL, 4 M in 1,4-dioxane). The mixture was stirred at room temperature for 1 h. The mixture was quenched with NaHCO₃ (sat. aq., 10 mL) and extracted with EtOAc (30 mL×3). The combined organic layers were concentrated in vacuo and purified by preparative TLC (eluent: DCM/MeOH=15/1) to afford (3S,5R)-1-(2-((1S*,2S*)-2-(3-chlorophenyl)cyclopropyl)-3-fluoro-1H-pyrrolo[3,2-c]pyridin-6-yl)-5-(6-cyclopropylimidazo[1,2-a]pyridin-2-yl)pyrrolidin-3-ol (1.7 mg, yield 38%) as a pale-yellow solid.

ESI-MS [M+H]+: 528.2. ¹H NMR (400 MHz, DMSO) δ 8.48 (s, 1H), 8.21 (s, 1H), 7.53 (d, J=3.1 Hz, 1H), 7.37-7.34 (m, 1H), 7.25 (s, 1H), 6.99-6.92 (m, 2H), 6.39-6.32 (m, 3H), 5.08 (s, 1H), 4.94-4.91 (m, 1H), 4.54-4.51 (m, 1H), 3.82-3.78 (m, 1H), 3.19-3.15 (m, 1H), 2.30-2.18 (m, 3H), 2.02-1.96 (m, 1H), 1.91-1.85 (m, 1H), 1.59-1.54 (m, 1H), 1.50-1.44 (m, 1H), 0.91-0.85 (m, 2H), 0.65-0.58 (m, 2H).

### Examples 57-59

### Synthesis of rac-1-(6-cyclopropyl-2-(((2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (I-57)

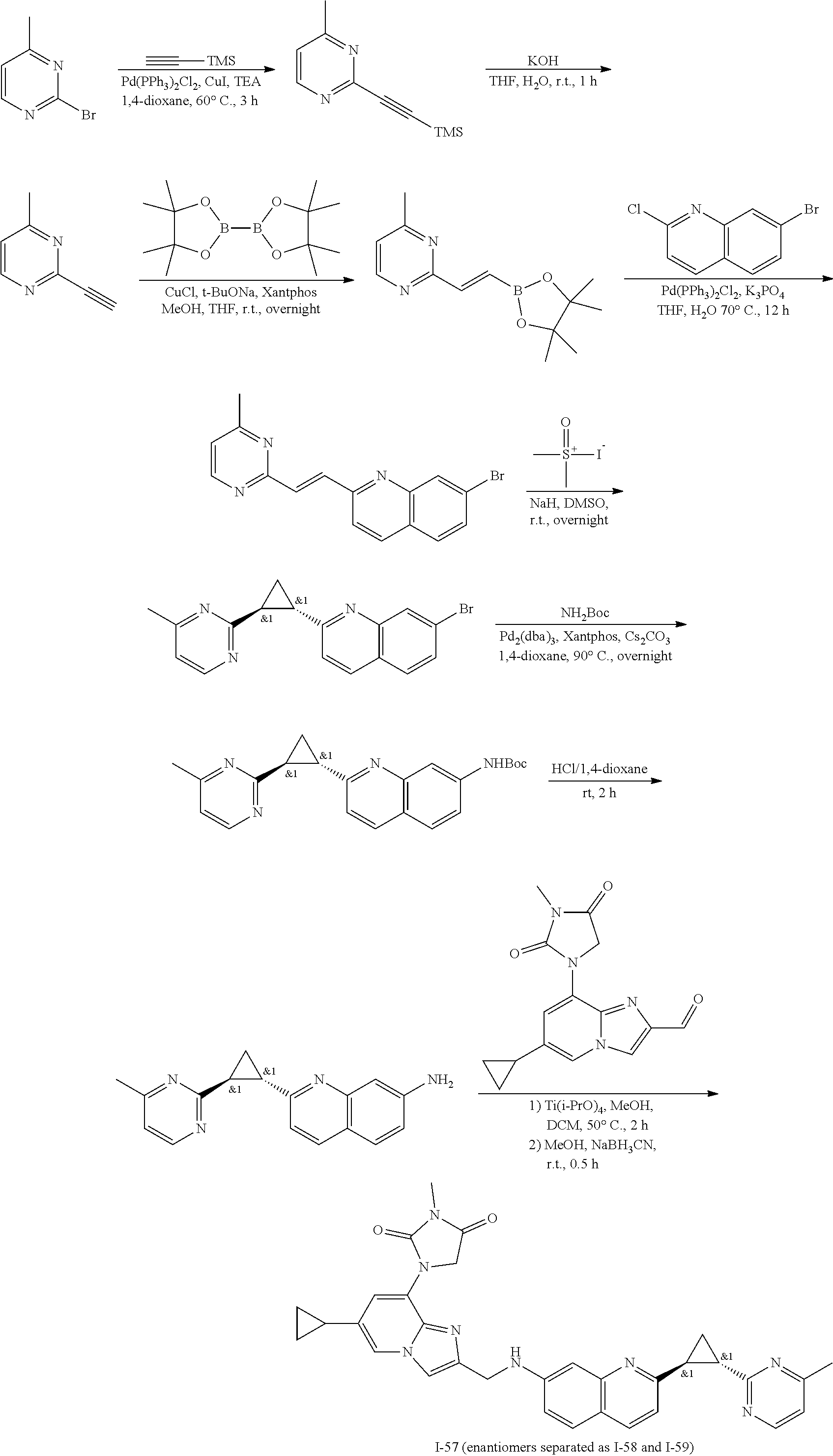

### Synthesis of 4-methyl-2-((trimethylsilyl)ethynyl)pyrimidine

To a mixture of 2-bromo-4-methylpyrimidine (6.0 g, 35 mmol) in 1,4-dioxane (90 mL) was added ethynyltrimethylsilane (6.8 g, 69 mmol), Pd(PPh₃)₂Cl₂ (0.73 g, 1.0 mmol), CuI (0.66 g, 3.5 mmol) and TEA (11 g, 110 mmol). The mixture was stirred at 60° C. for 3 h under N₂. The reaction mixture was filtered, washed with EtOAc (80 mL). The filtrate was concentrated in vacuo to give the crude, which was purified by column chromatography on silica gel (eluent: EtOAc/PE=0-10%) to afford 4-methyl-2-((trimethylsilyl)ethynyl)pyrimidine (5.8 g, yield 87%) as a brown solid.

ESI-MS [M+H]+: 191.2

### Synthesis of 2-ethynyl-4-methylpyrimidine

To a mixture of 4-methyl-2-((trimethylsilyl)ethynyl)pyrimidine (5.8 g, 30 mmol) in THF (80 mL) was added a solution KOH (3.4 g, 60 mmol) in H₂O (20 mL). The mixture was stirred at room temperature for 1 h. The reaction was diluted with H₂O (40 mL) and extracted with EtOAc (40 mL×3). The combined organic layers were washed with brine (50 mL), dried over Na₂SO₄, filtered, and concentrated in vacuo. The residue was purified by column chromatography on silica gel (eluent: EtOAc/PE=0-10%) to afford 2-ethynyl-4-methylpyrimidine (3.2 g, yield 90%) as pale-yellow solid.

ESI-MS [M+H]+: 119.2

### Synthesis of (E)-4-methyl-2-(2-(4,4,5,5-tetramethyl-1,3,2-dioxaborolan-2-yl)vinyl)pyrimidine

A mixture of CuCl (79 mg, 0.80 mmol), t-BuONa (154 mg, 1.6 mmol) and Xantphos (462 mg, 0.80 mmol) in in THF (150 mL) was stirred at room temperature for 0.5 h, a solution of 4,4,4′,4′,5,5,5′,5′-octamethyl-2,2′-bi(1,3,2-dioxaborolane) (6.9 g, 27 mmol) in THF (10 mL) was added thereto and stirred for another 10 min. Then a solution of 2-ethynyl-4-methylpyrimidine (3.2 g, 27 mmol) in THF (5 mL) was added, followed by addition of MeOH (4.8 g, 150 mmol). The resulting reaction mixture was stirred overnight at room temperature under N₂. The reaction mixture was filtered and concentrated in vacuo. The residue was diluted with H₂O (70 mL) and extracted with EtOAc (50 mL×3). The combined organic layers were washed with brine (50 mL), dried over Na₂SO₄, filtered and concentrated in vacuo. The reside was purified by column chromatography on silica gel (eluent: EtOAc/PE=0-10%) to afford (E)-4-methyl-2-(2-(4,4,5,5-tetramethyl-1,3,2-dioxaborolan-2-yl)vinyl)pyrimidine (6 g, yield 91%) as pale-yellow sticky oil. ESI-MS [M+H]+: 247.2

### Synthesis of (E)-7-bromo-2-(2-(4-methylpyrimidin-2-yl)vinyl)quinoline

To a solution of (E)-4-methyl-2-(2-(4,4,5,5-tetramethyl-1,3,2-dioxaborolan-2-yl)vinyl)pyrimidine (2.9 g, 12 mmol) and 7-bromo-2-chloroquinoline (2.9 g, 12 mmol) in THF (90 mL) and H₂O (9 mL) was added K₃PO₄ (7.6 g, 36 mmol) and Pd(PPh₃)₂Cl₂ (0.83 g, 1.2 mmol) under N₂. The mixture was stirred for 12 h at 70° C. under N₂. The reaction mixture was filtered and concentrated in vacuo. The residue was diluted with water (70 mL), and then extracted with DCM (100 mL×3). The combined organic layers were washed with brine (50 mL), dried over Na₂SO₄, filtered and concentrated in vacuo. The reside was purified by column chromatography on silica gel (eluent: MeOH/DCM=0-15%) to afford (E)-7-bromo-2-(2-(4-methylpyrimidin-2-yl)vinyl)quinoline (2.3 g, yield 59%) as white solid. ESI-MS [M+H]+: 326.2

### Synthesis of rac-7-bromo-2-((1S*,2S*)-2-(4-methylpyrimidin-2-yl)cyclopropyl)quinoline

To a mixture of trimethylsulfoxonium iodide (2.9 g, 13 mmol) in DMSO (30 mL) was added NaH (0.52 g, 13 mmol, 60% dispersion in mineral oil) at room temperature under N₂. The mixture was stirred at room temperature for 1 h. A solution of (E)-7-bromo-2-(2-(4-methylpyrimidin-2-yl)vinyl)quinoline (2.3 g, 7.1 mmol) in DMSO (5 mL) was added. The suspension solution was stirred at room temperature overnight. The reaction was quenched with NH₄Cl (Sat. aq., 30 mL), and then extracted with DCM (50 mL×3). The combined organic phase was washed with brine (50 mL), dried over Na₂SO₄, filtered, and concentrated in vacuo. The residue was purified by column chromatography on silica gel (eluent: EtOAc/PE=0-20%) to afford rac-7-bromo-2-((1S*,2S*)-2-(4-methylpyrimidin-2-yl)cyclopropyl)quinoline (0.75 g, yield 31%) as colorless sticky oil. ESI-MS [M+H]+: 340.2

### Synthesis of rac-tert-butyl (2-((1S*,2S*)-2-(4-methylpyrimidin-2-yl)cyclopropyl)quinolin-7-yl) carbamate

To a mixture of rac-7-bromo-2-((1S*,2S*)-2-(4-methylpyrimidin-2-yl)cyclopropyl)quinoline (400 mg, 1.2 mmol) and NH₂Boc (160 mg, 1.4 mmol) in 1,4-dioxane (30 mL) was added Pd₂(dba)₃ (110 mg, 0.12 mmol), Xantphos (133 mg, 0.23 mmol) and Cs₂CO₃ (1.2 g, 4.1 mmol). The reaction mixture was stirred at 90° C. overnight. The reaction was filtered and concentrated in vacuo. The residue was purified by column chromatography on silica gel (eluent: EtOAc/PE=0-40%) to afford rac-tert-butyl (2-((1S*,2S*)-2-(4-methylpyrimidin-2-yl)cyclopropyl)quinolin-7-yl)carbamate (420 mg, 93%) as a pale-yellow solid. ESI-MS [M+H]+: 377.2

### Synthesis of rac-2-((1S*,2S*)-2-(4-methylpyrimidin-2-yl)cyclopropyl)quinolin-7-amine

To a mixture of rac-tert-butyl (2-((1S*,2S*)-2-(4-methylpyrimidin-2-yl)cyclopropyl)quinolin-7-yl)carbamate (420 mg, 1.1 mmol) in 1,4-dioxane (20 mL) was added HCl (3 mL, 12 mmol, 4 M in 1,4-dioxane). The mixture was stirred at room temperature for 2 h. The reaction was concentrated in vacuo. The residue was treated with NH₃ (2 mL, 7M in MeOH) and stirred for 10 min. The solution was concentrated in vacuo and purified by column chromatography on silica gel (eluent: MeOH/DCM=0-6%) to afford rac-2-((1S*,2S*)-2-(4-methylpyrimidin-2-yl)cyclopropyl) quinolin-7-amine (290 mg, yield 95%) as yellow solid. ESI-MS [M+H]+: 277.2

### Synthesis of rac-1-(6-cyclopropyl-2-(((2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione

To a mixture of rac-2-((1S*,2S*)-2-(4-methylpyrimidin-2-yl)cyclopropyl)quinolin-7-amine (65 mg, 0.24 mmol) and 6-cyclopropyl-8-(3-methyl-2,4-dioxoimidazolidin-1-yl)imidazo[1,2-a]pyridine-2-carbaldehyde (70 mg, 0.24 mmol) in DCM (3 mL) and MeOH (3 mL) was added Ti(Oi-Pr)4 (205 mg. 0.72 mmol). The mixture was stirred at 50° C. for 2 h. The reaction solution was cooled to room temperature, MeOH (1 mL) was added followed by addition of NaBH₃CN (52 mg, 0.83 mmol). After stirring for another 0.5 h at room temperature. The reaction was diluted with water (30 mL) and extracted with EtOAc (30 mL×3). The combined organic layers were washed with brine (30 mL), dried over Na₂SO₄, concentrated in vacuo. The residue was purified by preparative TLC (eluent: 100% EA) to afford rac-1-(6-cyclopropyl-2-(((2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (25 mg, yield 19%) as a pale-yellow solid as a mixture of enantiomers.

ESI-MS [M+H]⁺: 559.3. ¹H NMR (400 MHz, DMSO) δ 8.51 (d, J=5.1 Hz, 1H), 8.25 (d, J=1.0 Hz, 1H), 7.91 (d, J=8.2 Hz, 1H), 7.76 (s, 1H), 7.57 (d, J=8.9 Hz, 1H), 7.35 (d, J=1.4 Hz, 1H), 7.17 (d, J=5.1 Hz, 1H), 7.11 (d, J=8.2 Hz, 1H), 7.05 (dd, J=8.8, 2.2 Hz, 1H), 6.80 (t, J=5.7 Hz, 1H), 6.76 (d, J=1.9 Hz, 1H), 4.95 (s, 2H), 4.48 (d, J=5.6 Hz, 2H), 2.98 (s, 3H), 2.72-2.65 (m, 2H), 2.41 (s, 3H), 1.96-1.89 (m, 1H), 1.77-1.68 (m, 2H), 0.95-0.90 (m, 2H), 0.66-0.62 (m, 2H).

The mixture was separated using chiral column separation [CHIRALPAK OZ-H, 0.46 cm×15 cm, MEOH/DEA=100/0.1, 1 mL/min, 35° C.) to give two enantiomers: 1-(6-cyclopropyl-2-(((2-((1R,2R)-2-(4-methylpyrimidin-2-yl)cyclopropyl)quinolin-7-yl)amino)methyl)imidazo[1,2-a]pyridin-8-yl)-3-methylimidazolidine-2,4-dione and 1-(6-cyclopropyl-2-(((2-((1S,2S)-2-(4-methylpyrimidin-2-yl)cyclopropyl)quinolin-7-yl)amino)methyl)imidazo[1,2-a]pyridin-8-yl)-3-methylimidazolidine-2,4-dione.

First eluting isomer (1-(6-cyclopropyl-2-(((2-((1R,2R)-2-(4-methylpyrimidin-2-yl)cyclopropyl)quinolin-7-yl)amino)methyl)imidazo[1,2-a]pyridin-8-yl)-3-methylimidazolidine-2,4-dione) (Example 58, I-58)

ESI-MS [M+H]+: 559.3. ¹H NMR (400 MHz, DMSO) δ 8.51 (d, J=5.1 Hz, 1H), 8.25 (s, 1H), 7.91 (d, J=8.2 Hz, 1H), 7.76 (s, 1H), 7.57 (d, J=8.8 Hz, 1H), 7.35 (d, J=1.4 Hz, 1H), 7.17 (d, J=5.1 Hz, 1H), 7.11 (d, J=8.2 Hz, 1H), 7.05 (dd, J=8.8, 2.2 Hz, 1H), 6.81 (t, J=5.7 Hz, 1H), 6.76 (d, J=1.8 Hz, 1H), 4.95 (s, 2H), 4.48 (d, J=5.6 Hz, 2H), 2.98 (s, 3H), 2.72-2.65 (m, 2H), 2.40 (s, 3H), 1.96-1.89 (m, 1H), 1.75-1.68 (m, 2H), 0.96-0.90 (m, 2H), 0.66-0.62 (m, 2H). RT=8.746 min, 99.75% e.e.

Second eluting isomer (1-(6-cyclopropyl-2-(((2-((1S,2S)-2-(4-methylpyrimidin-2-yl)cyclopropyl)quinolin-7-yl)amino)methyl)imidazo[1,2-a]pyridin-8-yl)-3-methylimidazolidine-2,4-dione) (Example 59, I-59)

ESI-MS [M+H]+: 559.3 ¹H NMR (400 MHz, DMSO) δ 8.51 (d, J=5.1 Hz, 1H), 8.25 (s, 1H), 7.91 (d, J=8.2 Hz, 1H), 7.76 (s, 1H), 7.57 (d, J=8.8 Hz, 1H), 7.35 (d, J=1.4 Hz, 1H), 7.17 (d, J=5.1 Hz, 1H), 7.12 (d, J=8.2 Hz, 1H), 7.05 (dd, J=8.8, 2.2 Hz, 1H), 6.81 (t, J=5.7 Hz, 1H), 6.76 (d, J=1.8 Hz, 1H), 4.95 (s, 2H), 4.48 (d, J=5.6 Hz, 2H), 2.98 (s, 3H), 2.73-2.64 (m, 2H), 2.41 (s, 3H), 1.96-1.89 (m, 1H), 1.77-1.68 (m, 2H), 0.96-0.89 (m, 2H), 0.66-0.62 (m, 2H). RT=11.89 min, 99.72% e.e.

### Example 60

### Synthesis of rac-1-(6-cyclopropyl-2-(((2-((1S*,2S*)-2-(4-methylpyrimidin-2-yl)cyclopropyl) quinazolin-7-yl)amino)methyl)imidazo[1,2-a]pyridin-8-yl)-3-methylimidazolidine-2,4-dione (I-60)

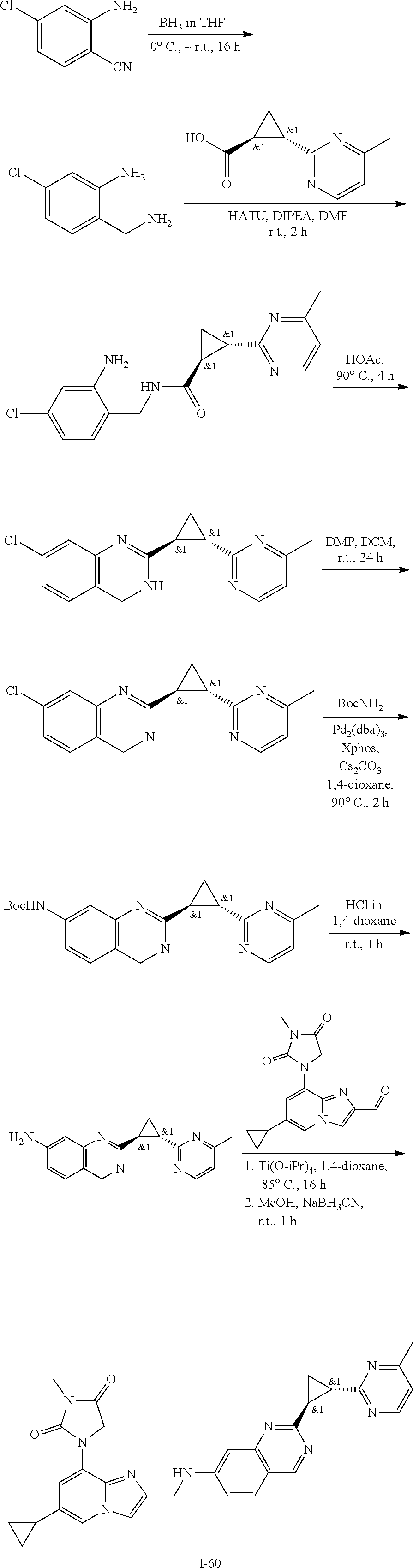

### Synthesis of 2-(aminomethyl)-5-chloroaniline

To a mixture of 2-amino-4-chlorobenzonitrile (300 mg, 1.97 mmol) in THF (10 mL) was added BH₃ (10 mL, 10 mmol, 1.0 M in THF) at 0° C. The reaction mixture was stirred at room temperature for 16 h. The reaction was quenched with EtOH (10 mL) and HCl (4 N in 1,4-dioxane, 10 mL). The resulting mixture was filtered and the precipitate was dried to give 5-(aminomethyl)-2-chloropyridin-4-amine as hydrochloric acid salt (0.45 g, crude) as a white solid. ESI-MS [M+H]⁺: 157.2.

### Synthesis of rac-(1S*,2S*)—N-(2-amino-4-chlorobenzyl)-2-(4-methylpyrimidin-2-yl)cyclopropane-1-carboxamide

A mixture of 2-(aminomethyl)-5-chloroaniline (150 mg crude), rac-(1S*,2S*)-2-(4-methylpyrimidin-2-yl)cyclopropane-1-carboxylic acid (110 mg, 0.62 mmol), HATU (251 mg, 0.66 mmol) and DIPEA (387 mg, 3.0 mmol) in dry DMF (5.0 mL) was stirred at room temperature for 2 h. The reaction was quenched with water (50 mL) and then extracted with EtOAc (30 mL×3). The combined organic layers were washed with brine (30 mL), dried over anhydrous Na₂SO₄, and then concentrated in vacuo to give the crude, which was purified by column chromatography on silica gel, eluting with 0˜6% MeOH/DCM to give rac-(1S*,2S*)—N-(2-amino-4-chlorobenzyl)-2-(4-methylpyrimidin-2-yl)cyclopropane-1-carboxamide (150 mg, 72% for 2 steps) as a white solid. ESI-MS [M+H]⁺: 317.2.

### Synthesis of rac-7-chloro-2-((1S*,2S*)-2-(4-methylpyrimidin-2-yl)cyclopropyl)-3,4-dihydroquinazoline

A solution of rac-(1S*,2S*)—N-(2-amino-4-chlorobenzyl)-2-(4-methylpyrimidin-2-yl)cyclopropane-1-carboxamide (140 mg, 0.44 mmol) in HOAc (5.0 mL) was stirred at 90° C. for 4 h. The reaction mixture was concentrated in vacuo to give rac-7-chloro-2-((1S*,2S*)-2-(4-methylpyrimidin-2-yl)cyclopropyl)-3,4-dihydroquinazoline (130 mg, crude) as a yellow solid, which was used in the next step without purification. ESI-MS [M+H]⁺: 299.1.

### Synthesis of rac-7-chloro-2-((1S*2S*)-2-(4-methylpyrimidin-2-yl)cyclopropyl)quinazoline

A mixture of rac-7-chloro-2-((1S*,2S*)-2-(4-methylpyrimidin-2-yl)cyclopropyl)-3,4-dihydroquinazoline (130 mg, crude) and DMP (560 mg, 1.3 mmol) in DCM (10 mL) was stirred at room temperature for 24 h under N₂. The reaction mixture was concentrated in vacuo to give the crude, which was purified by preparative TLC, eluting with 6% MeOH/DCM to give rac-7-chloro-2-((1S*,2S*)-2-(4-methylpyrimidin-2-yl)cyclopropyl)quinazoline (80 mg, 61% over 2 steps) as a yellow solid. ESI-MS [M+H]⁺: 297.2.

### Synthesis of rac-tert-butyl (2-((7S*,2S*)-2-(4-methylpyrimidin-2-yl)cyclopropyl)quinazolin-7-yl)carbamate

A mixture of rac-7-chloro-2-((1S*,2S*)-2-(4-methylpyrimidin-2-yl)cyclopropyl)quinazoline (60 mg, 0.20 mmol), BocNH₂ (31 mg, 0.26 mmol) and Cs₂CO₃ (170 mg, 0.52 mmol) in 1,4-dioxane (5.0 mL) was added Pd₂(dba)₃ (18 mg, 0.020 mmol) and X-Phos (19 mg, 0.040 mmol). The reaction mixture was stirred at 90° C. for 2 h under N₂. The resulting mixture was filtered through Celite® and the filter cake was washed with DCM/MeOH (10/1, 30 mL). The filtrate was concentrated in vacuo to give the crude, which was purified by preparative TLC, eluting with 10% MeOH/DCM to give rac-tert-butyl (2-((1S*,2S*)-2-(4-methylpyrimidin-2-yl)cyclopropyl)quinazolin-7-yl) carbamate (60 mg, 79%) as a yellow solid. ESI-MS [M+H]⁺: 378.2.

### Synthesis of rac-2-((1S*,2S*)-2-(4-methylpyrimidin-2-yl)cyclopropyl)quinazolin-7-amine

A mixture of rac-tert-butyl (2-((1S*,2S*)-2-(4-methylpyrimidin-2-yl)cyclopropyl) quinazolin-7-yl) carbamate (75 mg, 0.2 mmol) in HCl (4 N in 1,4-dioxane, 5.0 mL) was stirred at room temperature for 1 h. The reaction mixture was concentrated in vacuo to give rac-2-((1S*,2S*)-2-(4-methylpyrimidin-2-yl)cyclopropyl)quinazolin-7-amine as the hydrochloric acid salt (50 mg, crude) as a white solid, which was used in the next step without purification. ESI-MS [M+H]⁺: 278.2.

### Synthesis of rac-1-(6-cyclopropyl-2-(((2-((1S*,2S*)-2-(4-methylpyrimidin-2-yl)cyclopropyl) quinazolin-7-yl) amino)methyl)imidazo[1,2-a]pyridin-8-yl)-3-methylimidazolidine-2,4-dione

A mixture of rac-2-((1S*,2S*)-2-(4-methylpyrimidin-2-yl)cyclopropyl)quinazolin-7-amine (28 mg, 0.10 mmol), 6-cyclopropyl-8-(3-methyl-2,4-dioxoimidazolidin-1-yl)imidazo[1,2-a]pyridine-2-carbaldehyde (30 mg, 0.10 mmol) and Ti(O-iPr)₄ (140 mg, 0.49 mmol) in 1,4-dioxane (4.0 mL) was stirred at 85° C. for 16 h. After cooling to 0° C., the mixture was added NaBH₃CN (19 mg, 0.30 mmol) and MeOH (2.0 mL) subsequently. The resulting mixture was stirred at room temperature for 1 h. The reaction was quenched with water (20 mL) and then extracted with EtOAc (30 mL×3). The combined organic layers were washed with brine (20 mL), dried over anhydrous Na₂SO₄, and then concentrated in vacuo to give the crude, which was purified by Prep-HPLC to give rac-1-(6-cyclopropyl-2-(((2-((1S*,2S*)-2-(4-methylpyrimidin-2-yl)cyclopropyl)quinazolin-7-yl)amino)methyl)imidazo[1,2-a]pyridin-8-yl)-3-methylimidazolidine-2,4-dione (6.0 mg, 11%) as a light yellow solid.

ESI-MS [M+H]⁺: 560.2, ¹H NMR (400 MHz, DMSO) δ 8.94 (s, 1H), 8.52 (d, J=5.1 Hz, 1H), 8.46 (s, 1H), 8.25 (s, 1H), 7.79 (s, 1H), 7.69 (d, J=8.9 Hz, 1H), 7.47 (s, 1H), 7.35 (s, 1H), 7.18 (d, J=5.2 Hz, 1H), 7.11 (d, J=9.0 Hz, 1H), 6.64 (s, 1H), 4.92 (s, 2H), 4.51 (d, J=5.4 Hz, 2H), 2.97 (s, 3H), 2.76-2.60 (m, 2H), 2.41 (s, 3H), 1.94-1.93 (m, 1H), 1.78-1.71 (m, 2H), 0.93-0.92 (m, 2H), 0.64-0.63 (m, 2H).

### Example 61

### Synthesis of rac-1-(6-cyclopropyl-2-(((3-((1S*,2S*)-2-(4-methylpyrimidin-2-yl)cyclopropyl)-1,1-dioxido-4H-benzo[b][1,4]thiazin-6-yl)amino)methyl)imidazo[1,2-a]pyridin-8-yl)-3-methylimidazolidine-2,4-dione (I-61)

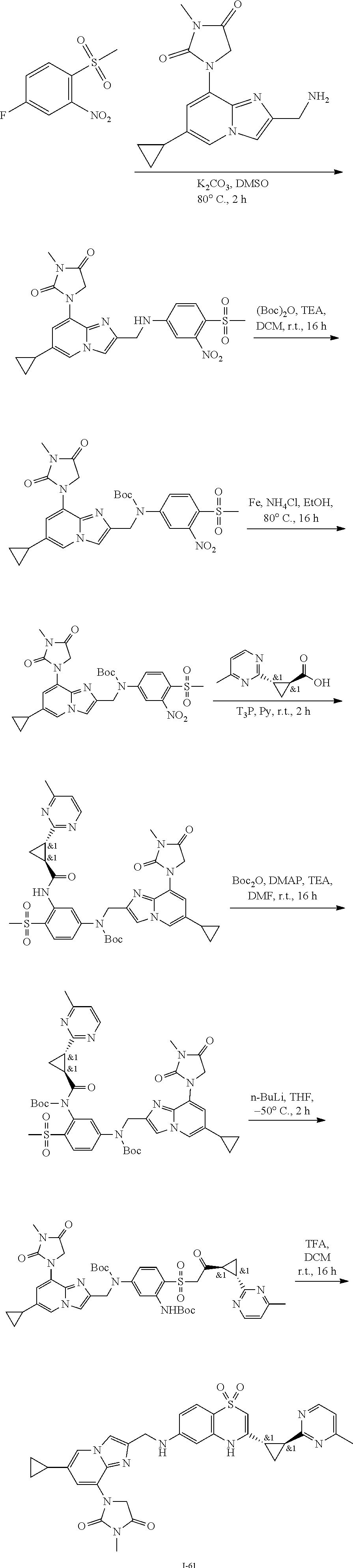

### Synthesis of 1-(6-cyclopropyl-2-(((4-(methylsulfonyl)-3-nitrophenyl)amino)methyl)imidazo[1,2-a]pyridin-8-yl)-3-methylimidazolidine-2,4-dione

To a mixture of 4-fluoro-1-(methylsulfonyl)-2-nitrobenzene (600 mg, 2.74 mmol) and 1-(2-(aminomethyl)-6-cyclopropylimidazo[1,2-a]pyridin-8-yl)-3-methylimidazolidine-2,4-dione (900 mg, 3 mmol) in DMSO (10 mL) was added K₂CO₃ (945 mg, 6.85 mmol) and the reaction mixture was stirred at 80° C. for 2 h under N₂. The reaction was cooled to room temperature, diluted with H₂O (50 mL) and extracted with EtOAc (50 mL×3). The combined organic layers were washed with brine (50 mL), dried over anhydrous Na₂SO₄ and concentrated in vacuo to give the crude, which was purified by silica gel column chromatography eluting with a gradient 0-5% MeOH in DCM to give 1-(6-cyclopropyl-2-(((4-(methylsulfonyl)-3-nitrophenyl)amino)methyl)imidazo[1,2-a]pyridin-8-yl)-3-methylimidazolidine-2,4-dione (1.3 g, 95%) as a brown oil. ESI-MS [M+H]+: 499.2.

### Synthesis of tert-butyl ((6-cyclopropyl-8-(3-methyl-2,4-dioxoimidazolidin-1-yl)imidazo[1,2-a]pyridin-2-yl)methyl)(4-(methylsulfonyl)-3-nitrophenyl)carbamate

To a solution of 1-(6-cyclopropyl-2-(((4-(methylsulfonyl)-3-nitrophenyl)amino)methyl)imidazo[1,2-a]pyridin-8-yl)-3-methylimidazolidine-2,4-dione (1.3 g, 2.6 mmol) in DCM (40 mL) was added di-tert-butyl dicarbonate (1.13 g, 5.2 mmol), DMAP (32 mg, 0.26 mmol) and TEA (791 mg, 7.83 mmol). The reaction mixture was stirred at room temperature for 16 h under N₂. The reaction was diluted with DCM (30 mL), washed with H₂O (20 mL×2) then brine (20 mL), dried over anhydrous Na₂SO₄ then concentrated in vacuo to give the crude, which was purified by silica gel column chromatography eluting with a gradient of 0-5% MeOH in DCM to give tert-butyl ((6-cyclopropyl-8-(3-methyl-2,4-dioxoimidazolidin-1-yl)imidazo[1,2-a]pyridin-2-yl)methyl)(4-(methyl sulfonyl)-3-nitrophenyl)carbamate (700 mg, 45%) as yellow oil. ESI-MS [M+H]+: 599.2.

### Synthesis of tert-butyl (3-amino-4-(methylsulfonyl)phenyl)((6-cyclopropyl-8-(3-methyl-2,4-dioxoimidazolidin-1-yl)imidazo[1,2-a]pyridin-2-yl)methyl)carbamate

A mixture of tert-butyl ((6-cyclopropyl-8-(3-methyl-2,4-dioxoimidazolidin-1-yl)imidazo[1,2-a]pyridin-2-yl)methyl)(4-(methylsulfonyl)-3-nitrophenyl)carbamate (600 mg, 1.0 mmol), NH₄Cl (583 mg, 11 mmol) and Fe powder (168 mg, 3 mmol) in EtOH (15 mL) was stirred at 80° C. for 2 h under N₂. The reaction mixture was filtered through Celite® and the filter cake was washed with DCM/MeOH (10/1, 50 mL). The filtrate was concentrated in vacuo to give tert-butyl (3-amino-4-(methylsulfonyl)phenyl)((6-cyclopropyl-8-(3-methyl-2,4-dioxoimidazolidin-1-yl)imidazo[1,2-a]pyridin-2-yl)methyl)carbamate (400 mg, crude) as yellow oil. ESI-MS [M+H]⁺: 569.2.

### Synthesis of rac-tert-butyl ((6-cyclopropyl-8-(3-methyl-2,4-dioxoimidazolidin-1-yl)imidazo[1,2-a]pyridin-2-yl)methyl)(3-((1S*,2S*)-2-(4-methylpyrimidin-2-yl)cyclopropane-1-carboxamido)-4-(methylsulfonyl)phenyl)carbamate

To a mixture of tert-butyl (3-amino-4-(methylsulfonyl)phenyl)((6-cyclopropyl-8-(3-methyl-2,4-dioxoimidazolidin-1-yl)imidazo[1,2-a]pyridin-2-yl)methyl)carbamate (400 mg, crude) and rac-(1S*,2S*)-2-(4-methylpyrimidin-2-yl)cyclopropane-1-carboxylic acid (125 mg, 0.7 mmol) in pyridine (10 mL) was added T3P (6.7 g, 10.5 mmol, 50% in EtOAc) dropwise at room temperature. The resulting reaction was stirred at room temperature for 2 h. The reaction was diluted with water (30 mL), extracted with EtOAc (30 mL×3). The combined organic layers were washed with brine (30 mL), dried over anhydrous Na₂SO₄ and concentrated in vacuo to give the crude, which was purified by preparative TLC (eluent: DCM/MeOH=20/1) to give rac-tert-butyl ((6-cyclopropyl-8-(3-methyl-2,4-dioxoimidazolidin-1-yl)imidazo[1,2-a]pyridin-2-yl)methyl)(3-((1S*,2S*)-2-(4-methylpyrimidin-2-yl)cyclopropane-1-carboxamido)-4-(methylsulfonyl)phenyl)carbamate (400 mg, 55% over 2 steps) as a yellow solid. ESI-MS [M+H]+: 729.3.

### Synthesis of rac-tert-butyl (3-((1S*,2S*)—N-(tert-butoxycarbonyl)-2-(4-methylpyrimidin-2-yl)cyclo propane-1-carboxamido)-4-(methylsulfonyl)phenyl)((6-cyclopropyl-8-(3-methyl-2,4-dioxo imidazolidin-1-yl)imidazo[1,2-a]pyridin-2-yl)methyl)carbamate

To a solution of rac-tert-butyl ((6-cyclopropyl-8-(3-methyl-2,4-dioxoimidazolidin-1-yl)imidazo[1,2-a]pyridin-2-yl)methyl)(3-((1S*,2S*)-2-(4-methylpyrimidin-2-yl)cyclopropane-1-carboxamido)-4-(methylsulfonyl)phenyl)carbamate (400 mg, 0.55 mmol) in DMF (10 mL) was added di-tert-butyl dicarbonate (240 mg, 1.1 mmol), DMAP (134 mg, 1.1 mmol) and TEA (167 mg, 1.65 mmol). The mixture was stirred at room temperature for 16 h under N₂. The reaction weas diluted with H₂O (50 mL), extracted with EtOAc (40 mL×3). The combined organic layers were washed with brine (40 mL), dried over anhydrous Na₂SO₄, and concentrated in vacuo to give the crude, which was purified by silica gel column chromatography, eluting with a gradient of 0-25% EtOAc in PE to give rac-tert-butyl (3-((1S*,2S*)—N-(tert-butoxycarbonyl)-2-(4-methylpyrimidin-2-yl)cyclopropane-1-carboxamido)-4-(methylsulfonyl)phenyl)((6-cyclopropyl-8-(3-methyl-2,4-dioxoimidazolidin-1-yl)imidazo[1,2-a]pyridin-2-yl)methyl)carbamate (400 mg, 88%) as a yellow solid. ESI-MS [M+H]⁺: 829.3

### Synthesis of rac-tert-butyl (3-((tert-butoxycarbonyl)amino)-4-((2-((1S*,2S*)-2-(4-methylpyrimidin-2-yl)cyclopropyl)-2-oxoethyl)sulfonyl)phenyl)((6-cyclopropyl-8-(3-methyl-2,4-dioxoimidazolidin-1-yl)imidazo[1,2-a]pyridin-2-yl)methyl)carbamate

To a solution of rac-tert-butyl (3-((1S*,2S*)—N-(tert-butoxycarbonyl)-2-(4-methylpyrimidin-2-yl)cyclopropane-1-carboxamido)-4-(methylsulfonyl)phenyl)((6-cyclopropyl-8-(3-methyl-2,4-dioxoimidazolidin-1-yl)imidazo[1,2-a]pyridin-2-yl)methyl)carbamate (200 mg, 0.24 mmol) in THF (10 mL) was added n-BuLi (2.4 M in hexane, 0.25 mL, 0.6 mmol) dropwise at −50° C. under N₂. The mixture was stirred at −50° C. for 2 h under N₂. The reaction was quenched with NH₄Cl (sat. aq., 20 mL) and extracted with EtOAc (30 mL×3). The combined organic layers were washed with brine (30 mL), dried over anhydrous Na₂SO₄, and concentrated in vacuo to give the crude, which was purified by preparative TLC (eluent: DCM/MeOH=15/1) to give rac-tert-butyl (3-((tert-butoxycarbonyl)amino)-4-((2-((1S*,2S*)-2-(4-methylpyrimidin-2-yl)cyclopropyl)-2-oxoethyl)sulfonyl)phenyl)((6-cyclopropyl-8-(3-methyl-2,4-dioxoimidazolidin-1-yl)imidazo[1,2-a]pyridin-2-yl)methyl)carbamate (60 mg, 30%) as a white solid. ESI-MS [M+H]⁺: 829.2

### Synthesis of rac-1-(6-cyclopropyl-2-(((3-((1S*,2S*)-2-(4-methylpyrimidin-2-yl)cyclopropyl)-1,1-dioxido-4H-benzo[b][1,4]thiazin-6-yl)amino)methyl)imidazo[1,2-a]pyridin-8-yl)-3-methylimidazolidine-2,4-dione

To a solution of rac-tert-butyl (3-((tert-butoxycarbonyl)amino)-4-((2-((1S*,2S*)-2-(4-methylpyrimidin-2-yl)cyclopropyl)-2-oxoethyl)sulfonyl)phenyl)((6-cyclopropyl-8-(3-methyl-2,4-dioxoimidazolidin-1-yl)imidazo[1,2-a]pyridin-2-yl)methyl)carbamate (60 mg, 0.072 mmol) in DCM (2 mL) was added TFA (1 mL). The reaction mixture was stirred at room temperature for 16 h under N₂. NaHCO₃ (sat. aq., 20 mL) was added and the mixture extracted with DCM (20 mL×3). The combined organic layers were concentrated in vacuo to give the crude, which was purified by preparative TLC (eluent: DCM/MeOH=10/1) to give rac-1-(6-cyclopropyl-2-(((3-((1S*,2S*)-2-(4-methylpyrimidin-2-yl)cyclopropyl)-1,1-dioxido-4H-benzo[b][1,4]thiazin-6-yl)amino)methyl)imidazo[1,2-a]pyridin-8-yl)-3-methylimidazolidine-2,4-dione (30 mg, 68%) as an off-white solid.

ESI-MS [M+H]⁺: 611.2, ¹H NMR (400 MHz, DMSO) δ 10.23 (s, 1H), 8.53 (d, J=5.1 Hz, 1H), 8.27 (s, 1H), 7.69 (s, 1H), 7.43 (d, J=8.9 Hz, 1H), 7.35 (s, 1H), 7.20 (d, J=5.1 Hz, 1H), 7.05 (s, 1H), 6.62 (d, J=8.9 Hz, 1H), 6.30 (s, 1H), 5.71 (s, 1H), 4.87 (s, 2H), 4.40 (s, 2H), 2.97 (s, 3H), 2.47-2.44 (m, 1H), 2.41 (s, 3H), 2.31-2.29 (m, 1H), 1.93-1.92 (m, 1H), 1.68-1.61 (m, 1H), 1.55-1.52 (m, 1H), 0.94-0.93 (m, 2H), 0.65-0.64 (m, 2H).

### Example 62

### Synthesis of rac-1-(6-cyclopropyl-2-(((3-((1S*,2S*)-2-(4-methylpyrimidin-2-yl)cyclopropyl)quinoxalin-6-yl)amino)methyl)imidazo[1,2-a]pyridin-8-yl)-3-methylimidazolidine-2,4-dione (I-62)

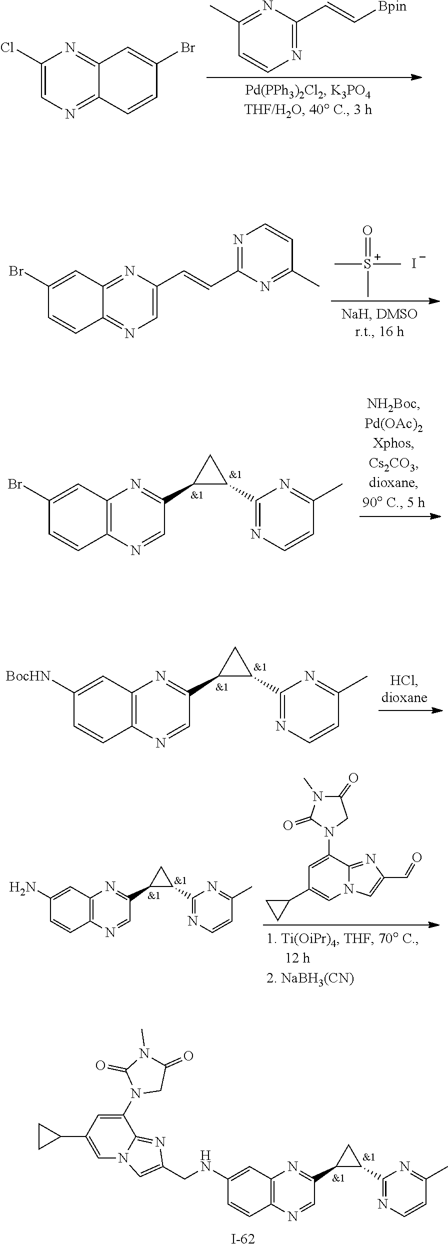

### Synthesis of (E)-7-bromo-2-(2-(4-methylpyrimidin-2-yl)vinyl)quinoxaline

To a mixture of 7-bromo-2-chloroquinoxaline (4.0 g, 16.5 mmol), (E)-4-methyl-2-(2-(4,4,5,5-tetramethyl-1,3,2-dioxaborolan-2-yl)vinyl)pyrimidine (3.7 g, 15 mmol) and K₃PO₄ (9.5 g, 45 mmol) in THF/water (100/10 mL) was added Pd(PPh₃)₂Cl₂ (526 mg, 0.75 mol). The resulting mixture was stirred at 40° C. for 3 h under N₂. The reaction was poured in water (100 mL) and extracted with EtOAc (100 mL×3). The combined organic layers were washed with brine (60 mL), dried over anhydrous Na₂SO₄, and concentrated in vacuo to give the crude, which was purified by column chromatography (eluent: MeOH/DCM=0˜3%) to give the (E)-7-bromo-2-(2-(4-methylpyrimidin-2-yl)vinyl)quinoxaline (2.0 g, 41%) as yellow solid. ESI-MS [M+H]⁺: 327.0.

### Synthesis of rac-7-bromo-2-((1S*2S*)-2-(4-methylpyrimidin-2-yl)cyclopropyl)quinoxaline

To a stirred solution of NaH (1.2 g, 60% in mineral oil, 30.5 mmol) in DMSO (30 mL) was added trimethylsulfoxonium iodide (6.7 g, 30.5 mmol) portion-wise and the mixture was stirred at room temperature for 1 h under N₂. A solution of (E)-7-bromo-2-(2-(4-methylpyrimidin-2-yl)vinyl)quinoxaline (2.0 g, 6.1 mmol) in DMSO (10 mL) was added slowly and the resulting reaction mixture was stirred at room temperature for 16 h. The heterogeneous mixture was poured into brine (200 mL) and extracted with EtOAc (3×100 mL). The combined organic layers were washed with brine (50 mL), dried over Na₂SO₄ then concentrated in vacuo to give the crude, which was purified by silica gel chromatography (eluent: MeOH/DCM=0˜3%) to give the rac-7-bromo-2-((1S*,2S*)-2-(4-methylpyrimidin-2-yl)cyclopropyl)quinoxaline (460 mg, 22%) as a yellow solid. ESI-MS [M+H]⁺: 341.0.

### Synthesis of rac-tert-butyl (3-((1S*,2S*)-2-(4-methylpyrimidin-2-yl)cyclopropyl)quinoxalin-6-yl)carbamate

To a stirred mixture of 7-bromo-2-(2-(4-methylpyrimidin-2-yl)cyclopropyl)quinoxaline (440 mg, 1.3 mmol), BocNH₂ (304 mg, 2.6 mmol), Xphos (124 mg, 0.26 mmol), Cs₂CO₃ (1.3 g, 3.9 mmol) in 1,4-dioxane (20 mL) was added Pd(OAc)₂ (29 mg, 0.13 mmol) and the reaction mixture was stirred at 90° C. for 5 h under N₂. The reaction mixture was filtered through Celite® and the filter cake was washed with DCM/MeOH (10/1, 30 mL). The filtrate was concentrated in vacuo to give the crude, which was purified by column chromatography (eluent: MeOH:DCM=0˜3%) to give rac-tert-butyl (3-((1S*,2S*)-2-(4-methylpyrimidin-2-yl)cyclopropyl)quinoxalin-6-yl)carbamate (400 mg, 82%) as a yellow solid. ESI-MS [M+H]⁺: 378.2.

### Synthesis of rac-3-((1S*,2S*)-2-(4-methylpyrimidin-2-yl)cyclopropyl)quinoxalin-6-amine

To a solution of rac-tert-butyl (3-((1S*,2S*)-2-(4-methylpyrimidin-2-yl)cyclopropyl)quinoxalin-6-yl)carbamate (400 mg, 1.1 mol) in 1,4-dioxane (10 mL) was added HCl (5 mL, 4M solution in 1,4-dioxane). The mixture was stirred at room temperature for 2 h. The reaction was quenched with NaHCO₃ (sat. aq., 50 mL) and extracted with EtOAc (50 mL×3). The combined organic layers were washed with brine (30 mL), dried over Na₂SO₄, concentrated in vacuo to give the crude, which was purified by column chromatography (eluent: MeOH/DCM=0˜5%) to afford rac-3-((1S*,2S*)-2-(4-methylpyrimidin-2-yl)cyclopropyl)quinoxalin-6-amine (120 mg, 39%) as yellow solid. ESI-MS [M+H]⁺: 278.1.

### Synthesis of rac-1-(6-cyclopropyl-2-(((3-((1S*,2S*)-2-(4-methylpyrimidin-2-yl)cyclopropyl) quinoxalin-6-yl)amino)methyl)imidazo[1,2-a]pyridin-8-yl)-3-methylimidazolidine-2,4-dione

To a mixture of rac-3-((1S*,2S*)-2-(4-methylpyrimidin-2-yl)cyclopropyl)quinoxalin-6-amine (55 mg, 0.2 mmol) and 6-cyclopropyl-8-(3-methyl-2,4-dioxoimidazolidin-1-yl)imidazo[1,2-a]pyridine-2-carbaldehyde (60 mg, 0.2 mol) in THF (10 mL) was added Ti(Oi-Pr)₄ (284 mg, 1 mmol) and the resulting mixture was stirred 12 h at 70° C. After the reaction was cooled to room temperature, MeOH (2 mL) and NaBH₃CN (38 mg, 0.6 mmol) was added and the reaction mixture was stirred at room temperature for 1 h. Water (50 mL) was added and extracted with EtOAc (50 mL×3). The combined organic layers were washed with brine (30 mL), dried over Na₂SO₄, then concentrated in vacuo to give the crude which was purified with preparative TLC (eluent: DCM/MeOH=40/1) to give rac-1-(6-cyclopropyl-2-(((3-((1S*,2S*)-2-(4-methylpyrimidin-2-yl)cyclopropyl)quinoxalin-6-yl)amino)methyl)imidazo[1,2-a]pyridin-8-yl)-3-methylimidazolidine-2,4-dione (36.5 mg, 33%) as a yellow solid.

ESI-MS [M+H]⁺: 560.2, ¹H NMR (400 MHz, DMSO) δ 8.55-8.50 (m, 2H), 8.27 (s, 1H), 7.80 (s, 1H), 7.70 (d, J=9.1 Hz, 1H), 7.36 (s, 1H), 7.30-7.28 (m, 2H), 7.19 (d, J=5.1 Hz, 1H), 7.14 (t, J=5.7 Hz, 1H), 6.77 (d, J=2.4 Hz, 1H), 4.94 (s, 2H), 4.52 (d, J=5.5 Hz, 2H), 2.98 (s, 3H), 2.84-2.78 (m, 1H), 2.77-2.72 (m, 1H), 2.42 (s, 3H), 1.95-1.90 (m, 1H), 1.80-1.77 (m, 2H), 0.96-0.90 (m, 2H), 0.67-0.61 (m, 2H)

### Example 63

### Synthesis of rac-1-(6-cyclopropyl-2-(((2-((1S*,2S*)-2-(4-methylpyrimidin-2-yl)cyclopropyl) pyrido[4,3-d]pyrimidin-7-yl)amino)methyl)imidazo[1,2-a]pyridin-8-yl)-3-methylimidazolidine-2,4-dione (I-63)

### Synthesis of 6-chloro-4-((1-(4-methoxyphenyl)ethyl)amino)nicotinamide

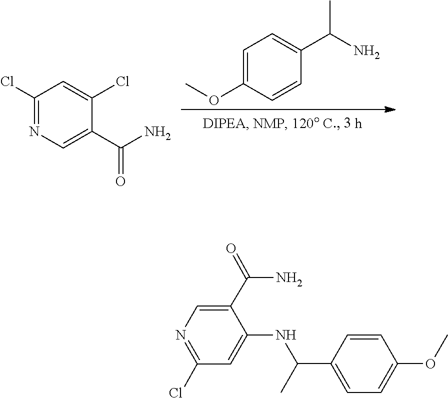 [SMILES: NC(=O)c1cnc(Cl)cc1Cl ; COc1ccc(C(C)N)cc1 ; COc1ccc(C(C)Nc2cc(Cl)ncc2C(N)=O)cc1]

A mixture of 4,6-dichloronicotinamide (2.3 g, 12 mmol), 1-(4-methoxyphenyl)ethan-1-amine (1.8 g, 12 mmol) and DIPEA (3.1 g, 24 mmol) in NMP (20 mL) was stirred at 120° C. for 3 h. Water (50 mL) was added and extracted with EtOAc (50 mL×3). The combined organic layers were washed with brine (50 mL), dried over anhydrous Na₂SO₄, and then concentrated in vacuo give 6-chloro-4-((1-(4-methoxyphenyl)ethyl)amino)nicotinamide (2.6 g, crude) as a yellow solid, which was used directly for the next step without further purification. ESI-MS [M+H]⁺: 306.2.

### Synthesis of 4-amino-6-chloronicotinonitrile

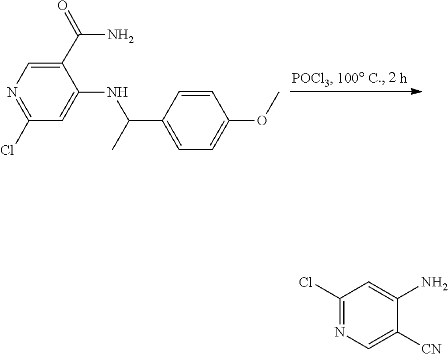 [SMILES: COc1ccc(C(C)Nc2cc(Cl)ncc2C(N)=O)cc1 ; N#Cc1cnc(Cl)cc1N]

A mixture of 6-chloro-4-((1-(4-methoxyphenyl)ethyl)amino)nicotinamide (2.6 g crude) in POCl₃ (15.0 mL) was stirred at 100° C. under N₂ for 2 h. The resulting mixture was concentrated in vacuo to give the residue, which was diluted with NaHCO₃ (sat. aq., 80 mL) and extracted with EtOAc (80 mL×3). The combined organic layers were washed with brine (80 mL), dried over Na₂SO₄ then concentrated in vacuo to give the crude, which was purified by column chromatography on silica gel, eluting with 0˜2% MeOH/DCM to give 4-amino-6-chloronicotinonitrile (1.0 g, 54% over 2 steps) as a yellow solid. ESI-MS [M+H]⁺: 154.2.

### Synthesis of 5-(aminomethyl)-2-chloropyridin-4-amine

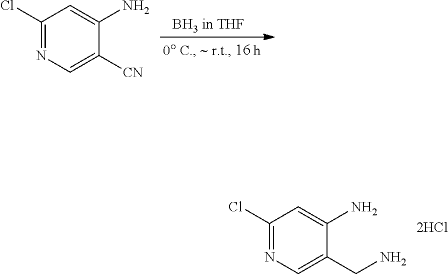 [SMILES: N#Cc1cnc(Cl)cc1N ; NCc1cnc(Cl)cc1N]

To a mixture of 4-amino-6-chloronicotinonitrile (1.2 g, 7.8 mmol) in THF (30 mL) was added BH₃ (1.0 N in THF, 39.0 mL, 39.0 mmol) at 0° C. The reaction mixture was stirred at room temperature for 16 h. The reaction was quenched with EtOH (20 mL) and HCl (4 N in 1,4-dioxane, 20 mL). The mixture was filtered, the filter cake was collected and dried to give 5-(aminomethyl)-2-chloropyridin-4-amine as hydrochloric acid salt (1.0 g, crude) as a white solid. ESI-MS [M+H]⁺: 158.2.

### Synthesis of rac-(1S*,2S*)—N-((4-amino-6-chloropyridin-3-yl)methyl)-2-(4-methylpyrimidin-2-yl) cyclopropane-1-carboxamide

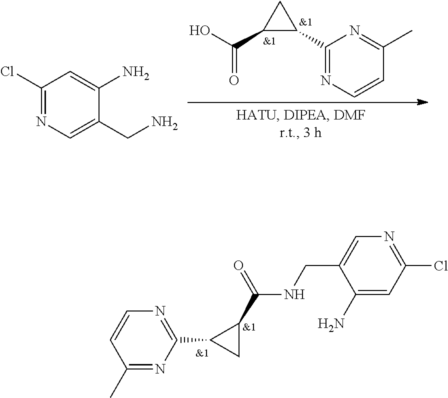 [SMILES: NCc1cnc(Cl)cc1N ; Cc1ccnc([C@H]2C[C@@H]2C(=O)O)n1 ; Cc1ccnc([C@H]2C[C@@H]2C(=O)NCc2cnc(Cl)cc2N)n1]

A mixture of (4-(17-azaneyl)-6-chloropyridin-3-yl)methanamine hydrochloride (500 mg crude), rac-(1S*,2S*)-2-(4-methylpyrimidin-2-yl)cyclopropane-1-carboxylic acid (392 mg, 2.2 mmol), HATU (1.67 g, 4.4 mmol) and DIPEA (1.4 g, 11 mmol) in dry DMF (15 mL) was stirred at room temperature for 3 h. The reaction was diluted with water (60 mL) and then extracted with EtOAc (50 mL×3). The combined organic layers were washed with brine (50 mL), dried over anhydrous Na₂SO₄, and then concentrated in vacuo to give the crude, which was purified by column chromatography on silica gel, eluting with 0˜6% MeOH/DCM to give rac-(1S*,2S*)—N-((4-amino-6-chloropyridin-3-yl)methyl)-2-(4-methylpyrimidin-2-yl)cyclopropane-1-carboxamide (0.35 g, 50%) as a white solid.

ESI-MS [M+H]⁺: 318.2.

### Synthesis of rac-7-chloro-2-((1S*,2S*)-2-(4-methylpyrimidin-2-yl)cyclopropyl)-3,4-dihydropyrido[4,3-d]pyrimidine

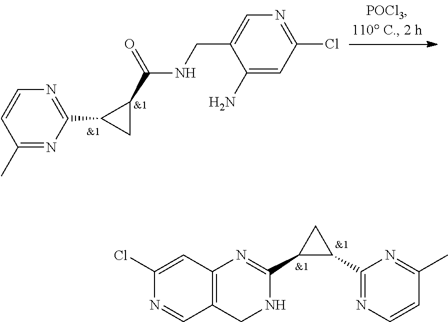 [SMILES: Cc1ccnc([C@H]2C[C@@H]2C(=O)NCc2cnc(Cl)cc2N)n1 ; Cc1ccnc([C@H]2C[C@@H]2C2=Nc3cc(Cl)ncc3CN2)n1]

A mixture of rac-(1S*,2S*)—N-((4-amino-6-chloropyridin-3-yl)methyl)-2-(4-methylpyrimidin-2-yl) cyclopropane-1-carboxamide (350 mg, 1.1 mmol) in POCl₃ (5.0 mL) was stirred at 110° C. for 2 h. The reaction mixture was concentrated in vacuo to give the crude, which was purified by preparative TLC, eluting with 10% MeOH/DCM to give rac-7-chloro-2-((1S*,2S*)-2-(4-methylpyrimidin-2-yl)cyclopropyl)-3,4-dihydropyrido[4,3-d]pyrimidine (300 mg, crude) as a yellow solid. ESI-MS [M+H]⁺: 300.1.

### Synthesis of rac-7-chloro-2-((1S*,2S*)-2-(4-methylpyrimidin-2-yl)cyclopropyl)pyrido[4,3-d]pyrimidine

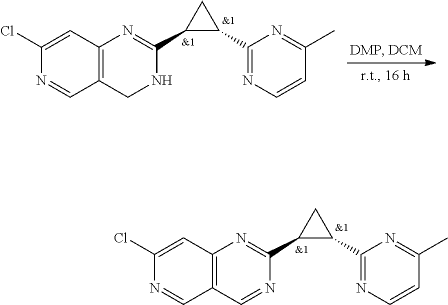 [SMILES: Cc1ccnc([C@H]2C[C@@H]2C2=Nc3cc(Cl)ncc3CN2)n1 ; Cc1ccnc([C@H]2C[C@@H]2c2ncc3cnc(Cl)cc3n2)n1]

A mixture of rac-7-chloro-2-((1S*,2S*)-2-(4-methylpyrimidin-2-yl)cyclopropyl)-3,4-dihydropyrido [4,3-d]pyrimidine (300 mg, 1.0 mmol) and DMP (0.85 g, 2.0 mmol) in DCM (10 mL) was stirred at room temperature for 16 h under N₂. The reaction mixture was concentrated in vacuo to give the crude, which was purified by preparative TLC, eluting with 10% MeOH/DCM to give rac-7-chloro-2-((1S*,2S*)-2-(4-methylpyrimidin-2-yl)cyclopropyl)pyrido[4,3-d]pyrimidine (180 mg, 61%) as a yellow solid. ESI-MS [M+H]⁺: 298.2.

### Synthesis of rac-1-(6-cyclopropyl-2-(((2-((1S*,2S*)-2-(4-methylpyrimidin-2-yl)cyclopropyl)pyrido[4,3-d] pyrimidin-7-yl)amino)methyl)imidazo[1,2-a]pyridin-8-yl)-3-methylimidazolidine-2,4-dione

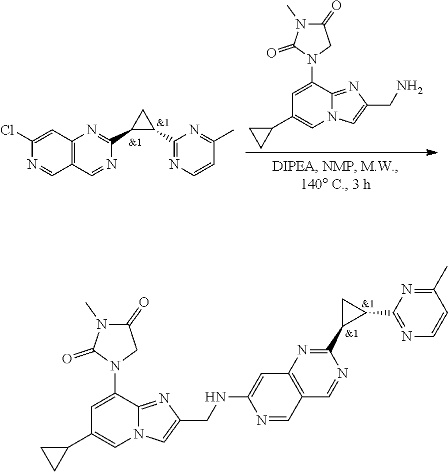

A mixture of rac-7-chloro-2-((1S*,2S*)-2-(4-methylpyrimidin-2-yl)cyclopropyl)pyrido[4,3-d] pyrimidine (50 mg, 0.17 mmol), 1-(2-(aminomethyl)-6-cyclopropylimidazo[1,2-a]pyridin-8-yl)-3-methylimidazolidine-2,4-dione (50 mg, 0.17 mmol) and DIPEA (66 mg, 0.51 mmol) in NMP (3 mL) was stirred in a sealed tube. After degassing with N₂ for 1 min, the resulting mixture was irradiated in a microwave reactor at 140° C. for 3 h. Then reaction was quenched with water (20 mL) and extracted with EtOAc (30 mL×3). The combined organic layers were washed with brine (30 mL), dried over anhydrous Na₂SO₄, and then concentrated in vacuo to give the crude, which was purified by Prep-HPLC to give rac-1-(6-cyclopropyl-2-(((2-((1S*,2S*)-2-(4-methylpyrimidin-2-yl)cyclopropyl)pyrido[4,3-d]pyrimidin-7-yl)amino)methyl)imidazo[1,2-a]pyridin-8-yl)-3-methylimidazolidine-2,4-dione (16 mg, 17%) as a yellow solid.

ESI-MS [M+H]⁺: 561.2, ¹H NMR (400 MHz, DMSO) δ 9.16 (s, 1H), 8.99 (s, 1H), 8.53 (d, J=5.1 Hz, 1H), 8.23 (s, 1H), 7.95 (t, J=5.9 Hz, 1H), 7.75 (s, 1H), 7.34 (d, J=1.4 Hz, 1H), 7.20 (d, J=5.1 Hz, 1H), 6.49 (s, 1H), 4.91 (s, 2H), 4.63 (s, 2H), 2.97 (s, 3H), 2.82-2.63 (m, 2H), 2.41 (s, 3H), 1.96-1.98 (m, 1H), 1.88-1.69 (m, 2H), 1.00-0.85 (m, 2H), 0.71-0.52 (m, 2H).

### Examples 64-66

### Synthesis of rac-6-(((6-cyclopropylimidazo[1,2-a]pyridin-2-yl)methyl)amino)-3-((1S*,2S*)-2-(4-methylpyrimidin-2-yl)cyclopropyl)-4H-benzo[e][1,2,4]thiadiazine 1,1-dioxide (I-64)

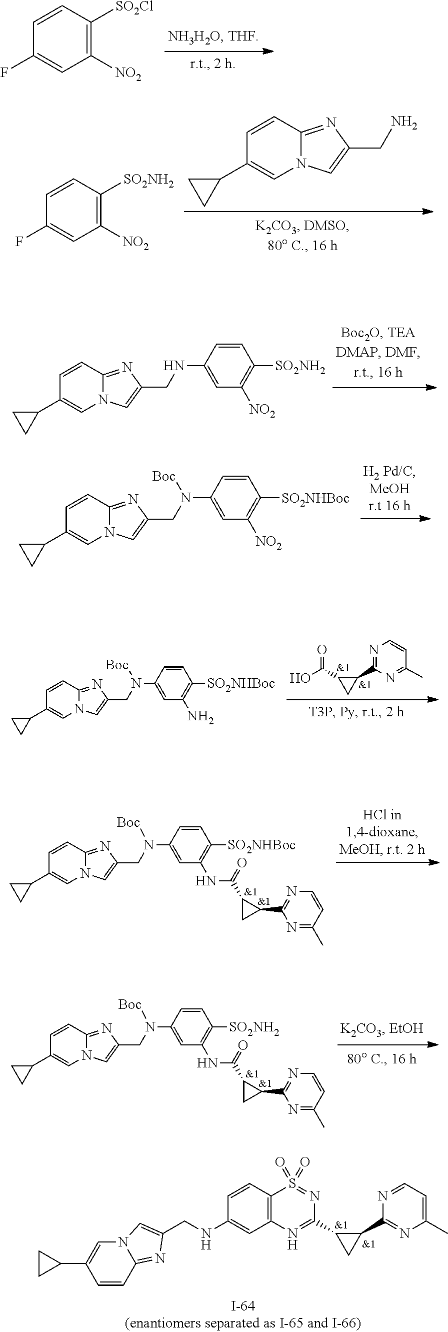

### Synthesis of 4-fluoro-2-nitrobenzenesulfonamide

To a mixture of 4-fluoro-2-nitrobenzenesulfonyl chloride (500 mg, 2.1 mmol) in THF (5 mL) was added NH₃/H₂O (1 mL. conc.). The reaction mixture was stirred at room temperature for 2 h then concentrated to give 4-fluoro-2-nitrobenzenesulfonamide (460 mg, quant.) as a yellow solid. ESI-MS [M+H]+: 221.2.

### Synthesis of 4-(((6-cyclopropylimidazo[1,2-a]pyridin-2-yl)methyl)amino)-2-nitrobenzenesulfonamide

A mixture of 4-fluoro-2-nitrobenzenesulfonamide (460 mg, 2.1 mmol), (6-cyclopropylimidazo[1,2-a]pyridin-2-yl)methanamine (430 mg, 2.3 mmol) and K₂CO₃ (725 mg, 5.25 mmol) in DMSO (10 mL) was stirred at 80° C. for 16 h under N₂. The reaction was cooled to room temperature, diluted with H₂O (30 mL) and extracted with EtOAc (30 mL×3). The combined organic layers were washed with brine (30 mL), dried over anhydrous Na₂SO₄, and concentrated in vacuo give the crude, which was purified with silica gel column chromatography eluting with a gradient of 0-40% EtOAc in PE to give 4-(((6-cyclopropylimidazo[1,2-a]pyridin-2-yl)methyl)amino)-2-nitrobenzene sulfonamide (500 mg, 62%) as a yellow solid. ESI-MS [M+H]+: 388.1.

### Synthesis of tert-butyl (4-(N-(tert-butoxycarbonyl)sulfamoyl)-3-nitrophenyl)((6-cyclopropylimidazo[1,2-a]pyridin-2-yl)methyl)carbamate

To a mixture of 4-(((6-cyclopropylimidazo[1,2-a]pyridin-2-yl)methyl)amino)-2-nitrobenzenesulfonamide (500 mg, 1.29 mmol) in DMF (10 mL) was added di-tert-butyl dicarbonate (562 mg, 2.58 mmol), DMAP (315 mg, 2.58 mmol) and TEA (391 mg, 3.87 mmol). The mixture was stirred at room temperature for 16 h under N₂. The reaction was diluted with water (30 mL) and extracted with EtOAc (50 mL×3). The combined organic layers were washed with brine (40 mL), dried over anhydrous Na₂SO₄, and concentrated in vacuo to give the crude product, which was purified by silica gel column chromatography, eluting with a gradient of 0-4% MeOH in DCM to give tert-butyl (4-(N-(tert-butoxycarbonyl)sulfamoyl)-3-nitrophenyl)((6-cyclopropylimidazo[1,2-a]pyridin-2-yl)methyl) carbamate (300 mg, 40%) as a yellow solid. ESI-MS [M+H]⁺: 588.2.

### Synthesis of tert-butyl (3-amino-4-(N-(tert-butoxycarbonyl)sulfamoyl)phenyl)((6-cyclopropyl imidazo[1,2-a]pyridin-2-yl)methyl)carbamate

To a mixture of tert-butyl (4-(N-(tert-butoxycarbonyl)sulfamoyl)-3-nitrophenyl)((6-cyclopropylimidazo[1,2-a]pyridin-2-yl)methyl)carbamate (300 mg, 0.51 mmol) in MeOH (5 mL) was added Pd/C (100 mg). The reaction mixture was stirred at room temperature for 16 h under H₂. The reaction mixture was filtered through Celite® and the filter cake was washed with DCM/MeOH (10/1, 30 mL). The filtrate was concentrated in vacuo to give the crude, which was purified by preparative TLC (eluent: DCM/MeOH=10/1) to give tert-butyl (3-amino-4-(N-(tert-butoxycarbonyl) sulfamoyl)phenyl)((6-cyclopropylimidazo[1,2-a]pyridin-2-yl)methyl)carbamate (170 mg, 60%) as a white solid. ESI-MS [M+H]+: 558.3.

### Synthesis of rac-tert-butyl (4-(N-(tert-butoxycarbonyl)sulfamoyl)-3-((1S*,2S*)-2-(4-methylpyrimidin-2-yl)cyclopropane-1-carboxamido)phenyl)((6-cyclopropylimidazo[1,2-a]pyridin-2-yl)methyl) carbamate

To a mixture of tert-butyl (3-amino-4-(N-(tert-butoxycarbonyl)sulfamoyl)phenyl)((6-cyclopropylimidazo[1,2-a]pyridin-2-yl)methyl)carbamate (170 mg, 0.3 mmol), rac-(1S*,2S*)-2-(4-methylpyrimidin-2-yl)cyclopropane-1-carboxylic acid (53 mg, 0.3 mmol) in pyridine (3 mL) was added T3P (1.9 g, 3 mmol, 50% in EtOAc) dropwise at room temperature under N₂. The mixture was stirred at room temperature for 2 h, then diluted with water (30 mL) and extracted with EtOAc (30 mL×3). The combined organic layers were washed with brine (30 mL), dried over anhydrous Na₂SO₄, and concentrated in vacuo to give the crude, which was purified by preparative TLC (eluent: DCM/MeOH=10/1) to give rac-tert-butyl (4-(N-(tert-butoxycarbonyl)sulfamoyl)-3-((1S*,2S*)-2-(4-methyl pyrimidin-2-yl)cyclopropane-1-carboxamido)phenyl)((6-cyclopropylimidazo[1,2-a]pyridin-2-yl)methyl)carbamate (100 mg, 46%) as a white solid. ESI-MS [M+H]+: 718.2.

### Synthesis of rac-(1S*,2S*)—N-(5-(((6-cyclopropylimidazo[1,2-a]pyridin-2-yl)methyl)amino)-2-sulfamoylphenyl)-2-(4-methylpyrimidin-2-yl)cyclopropane-1-carboxamide

To a mixture of rac-tert-butyl (4-(N-(tert-butoxycarbonyl)sulfamoyl)-3-((1S*,2S*)-2-(4-methylpyrimidin-2-yl)cyclopropane-1-carboxamido)phenyl)((6-cyclopropylimidazo[1,2-a]pyridin-2-yl)methyl)carbamate (100 mg, 0.14 mmol) in MeOH (2 mL) was added HCl (4M solution in 1,4-dioxane, 2 mL). The reaction mixture was stirred at room temperature for 2 h. The reaction mixture was concentrated in vacuo to give rac-(1S*,2S*)—N-(5-(((6-cyclopropylimidazo[1,2-a]pyridin-2-yl)methyl)amino)-2-sulfamoylphenyl)-2-(4-methylpyrimidin-2-yl)cyclopropane-1-carboxamide (72 mg, crude) as a yellow solid, which was used without further purification. ESI-MS [M+H]⁺: 518.2

### Synthesis of rac-6-(((6-cyclopropylimidazo[1,2-a]pyridin-2-yl)methyl)amino)-3-((1S*,2S*)-2-(4-methylpyrimidin-2-yl)cyclopropyl)-4H-benzo[e][1,2,4]thiadiazine 1,1-dioxide

To a solution of rac-(1S*,2S*)—N-(5-(((6-cyclopropylimidazo[1,2-a]pyridin-2-yl)methyl)amino)-2-sulfamoylphenyl)-2-(4-methylpyrimidin-2-yl)cyclopropane-1-carboxamide (72 mg, 0.14 mmol) in EtOH (5 mL) was added K₂CO₃ (72 mg, 0.52 mmol). The reaction mixture was stirred at 80° C. for 16 h under N₂. The reaction was cooled to room temperature, diluted with water (20 mL) and extracted with EtOAc (10 mL×3). The combined organic layers were washed with brine (20 mL), dried over anhydrous Na₂SO₄, and concentrated in vacuo to give the crude product, which was purified by preparative TLC (eluent: DCM/MeOH=10/1) to give rac-6-(((6-cyclopropylimidazo[1,2-a]pyridin-2-yl)methyl)amino)-3-((1S*,2S*)-2-(4-methylpyrimidin-2-yl)cyclopropyl)-4H-benzo[e][1,2,4]thiadiazine 1,1-dioxide (32 mg, 45%) as a yellow solid as a mixture of enantiomers.

ESI-MS [M+H]⁺: 500.2. Purity: 97.14 (214 nm), 96.73 (254 nm), ¹H NMR (400 MHz, DMSO) δ 11.94 (s, 1H), 8.55 (d, J=5.1 Hz, 1H), 8.29 (s, 1H), 7.61 (s, 1H), 7.42-7.36 (m, 2H), 7.23 (d, J=5.1 Hz, 2H), 6.96 (dd, J=9.3, 1.7 Hz, 1H), 6.72 (dd, J=8.8, 2.1 Hz, 1H), 6.27 (d, J=1.8 Hz, 1H), 4.38 (d, J=5.7 Hz, 2H), 2.66-2.61 (m, 1H), 2.47-2.45 (m, 1H), 2.42 (s, 3H), 1.89-1.88 (m, 1H), 1.70-1.65 (m, 2H), 0.93-0.87 (m, 2H), 0.69-0.62 (m, 2H).

The mixture was separated using SFC (Daicel CHIRALPAK OZ-H 20×250 mm, 5.0 μm, 30/70 MeOH (0.2% NH₄OH)/CO₂, 50 g/min, 35° C.) to give two enantiomers: 6-(((6-cyclopropylimidazo[1,2-a]pyridin-2-yl)methyl)amino)-3-((1S,2S)-2-(4-methylpyrimidin-2-yl)cyclopropyl)-4H-benzo[e][1,2,4]thiadiazine 1,1-dioxide and 6-(((6-cyclopropylimidazo[1,2-a]pyridin-2-yl)methyl)amino)-3-((1R,2R)-2-(4-methylpyrimidin-2-yl)cyclopropyl)-4H-benzo[e][1,2,4]thiadiazine 1,1-dioxide

First Eluting Isomer (Example 65, I-65)

ESI-MS (M+H)+: 500.2. ¹H NMR (400 MHz, MeOD) δ 8.46 (d, J=5.2 Hz, 1H), 8.12 (s, 1H), 7.58 (s, 1H), 7.50 (d, J=8.8 Hz, 1H), 7.37 (d, J=9.3 Hz, 1H), 7.17 (d, J=5.2 Hz, 1H), 7.06 (dd, J=9.4, 1.6 Hz, 1H), 6.75 (dd, J=8.8, 2.2 Hz, 1H), 6.25 (d, J=2.1 Hz, 1H), 4.48 (s, 2H), 2.91-2.80 (m, 1H), 2.50-2.47 (m, 1H), 2.47 (s, 3H), 1.93-1.83 (m, 2H), 1.79-1.74 (m, 1H), 0.96-0.94 (m, 2H), 0.69-0.67 (m, 2H). RT=1.442 min, 98.36 e.e.

Second Eluting Isomer (Example 66, I-66)

ESI-MS (M+H)+: 500.2. ¹H NMR (400 MHz, MeOD) δ 8.46 (d, J=5.2 Hz, 1H), 8.12 (s, 1H), 7.58 (s, 1H), 7.50 (d, J=8.8 Hz, 1H), 7.37 (d, J=9.3 Hz, 1H), 7.17 (d, J=5.2 Hz, 1H), 7.06 (dd, J=9.4, 1.6 Hz, 1H), 6.75 (dd, J=8.8, 2.2 Hz, 1H), 6.25 (d, J=2.1 Hz, 1H), 4.48 (s, 2H), 2.91-2.80 (m, 1H), 2.50-2.47 (m, 1H), 2.47 (s, 3H), 1.93-1.83 (m, 2H), 1.79-1.74 (m, 1H), 0.96-0.94 (m, 2H), 0.69-0.67 (m, 2H). RT=1.652 min, 98.24% e.e.

### Example 67

### Synthesis of rac-1-(6-cyclopropyl-2-((2R,4S)-4-hydroxy-1-(4-methoxy-2-((1S*,2S*)-2-(4-methylpyrimidin-2-yl)cyclopropyl)quinolin-7-yl)pyrrolidin-2-yl)imidazo[1,2-a]pyridin-8-yl)-3-methylimidazolidine-2,4-dione (I-67)

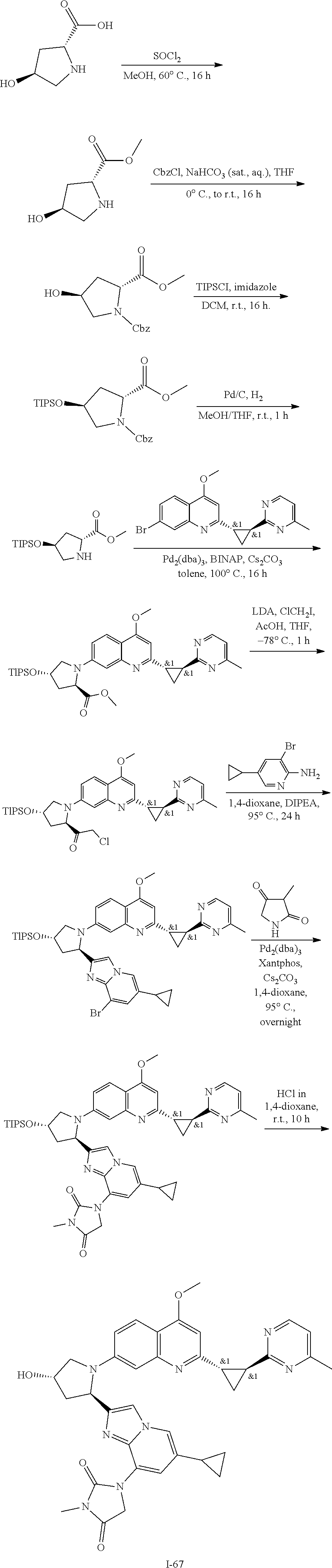

### Synthesis of methyl (2R,4S)-4-hydroxypyrrolidine-2-carboxylate

To a solution of (2R,4S)-4-hydroxypyrrolidine-2-carboxylic acid (5 g, 38.2 mmol) in MeOH (50 mL) was added SOCl₂ (9.0 g, 76.4 mmol) dropwise at 0° C. and the mixture was stirred at 60° C. for 16 h. The reaction mixture was cooled to room temperature then quenched with NaHCO₃ (sat. aq., 100 mL) and extracted with DCM (50 mL×3). The combined organic layers were washed with brine (50 mL), dried over anhydrous Na₂SO₄, and concentrated in vacuo to give the crude product, which was purified by colunm chromatography (eluent: DCM/MeOH=20/1) to give methyl (2R,4S)-4-hydroxypyrrolidine-2-carboxylate (5.0 g, 900) as a yellow oil. ESI-MS [M+H]+: 146.2.

### Synthesis of 1-benzyl 2-methyl (2R,4S)-4-hydroxypyrrolidine-1,2-dicarboxylate

To a solution of methyl (2R,4S)-4-hydroxypyrrolidine-2-carboxylate (5.0 g, 34.5 mmol) in THF (50 mL) and NaHCO₃ (sat. aq., 50 mL) was added a solution of CbzCl (5.9 g, 34.5 mmol) in THF (50 mL) at 0° C. The mixture was stirred at room temperature for 16 h. Water (100 mL) was added and the mixture was extracted with EtOAc (50 mL×3). The combined organic layers were washed with brine (100 mL), dried over anhydrous Na₂SO₄ and concentrated in vacuo to give the crude product, which was purified by column chromatography (eluent: PE/EtOAc=3/1) to give 1-benzyl 2-methyl (2R,4S)-4-hydroxypyrrolidine-1,2-dicarboxylate (7.8 g, 81%) as a yellow oil. ESI-MS [M+H]⁺: 280.1.

### Synthesis of 1-benzyl 2-methyl (2R,4S)-4-((triisopropylsilyl)oxy)pyrrolidine-1,2-dicarboxylate

A mixture of 1-benzyl 2-methyl (2R,4S)-4-hydroxypyrrolidine-1,2-dicarboxylate (7.8 g, 28.0 mmol), TIPSCl (5.4 g, 28.0 mmol) and imidazole (1.9 g, 28.0 mmol) in DCM (100 mL) was stirred at room temperature for 16 h. The reaction mixture was concentrated in vacuo to give the crude, which was purified by column chromatography (eluent: PE/EtOAc=5/1) to give 1-benzyl 2-methyl (2R,4S)-4-((triisopropylsilyl)oxy)pyrrolidine-1,2-dicarboxylate (5.8 g, 48%) as a yellow oil. ESI-MS [M+H]+: 436.2.

### Synthesis of methyl (2R,4S)-4-((triisopropylsilyl)oxy)pyrrolidine-2-carboxylate

A mixture of 1-benzyl 2-methyl (2R,4S)-4-((triisopropylsilyl)oxy)pyrrolidine-1,2-dicarboxylate (5.8 g, 13.3 mmol) and Pd/C (580 mg) in MeOH/THF (50 mL/50 mL) was stirred at room temperature for 1 h under H₂. The mixture was filtered through Celite® and the filter cake was washed with DCM/MeOH (10/1, 100 mL). The filtrate was concentrated in vacuo to give the crude, which was purified by column chromatography (eluent: DCM/MeOH=20/1) to give methyl (2R,4S)-4-((triiso-propylsilyl)oxy)pyrrolidine-2-carboxylate (3.2 g, 80%) as a white solid. ESI-MS [M+H]+: 302.2.

### Synthesis of rac-methyl (2R,4S)-1-(4-methoxy-2-((1S*,2S*)-2-(4-methylpyrimidin-2-yl)cyclopropyl)quinolin-7-yl)-4-((triisopropylsilyl)oxy)pyrrolidine-2-carboxylate

To a mixture of rac-7-bromo-4-methoxy-2-((1S*,2S*)-2-(4-methylpyrimidin-2-yl)cyclopropyl)quinoline (2 g, 5.4 mmol) and methyl (2R,4S)-4-((triisopropylsilyl)oxy)pyrrolidine-2-carboxylate (2.6 g, 8.7 mmol) in toluene (80 mL) was added Pd₂(dba)₃ (494 mg, 0.54 mmol), BINAP (684 mg, 1.1 mmol) and Cs₂CO₃ (5.2 g, 16 mmol). The mixture was stirred at 100° C. for 16 h under N₂. The reaction was filtered and concentrated in vacuo. The residue was quenched with H₂O (50 mL) then extracted with EtOAc (50 mL×3). The combined organic layers were washed with brine (50 mL), dried over Na₂SO₄, filtered and concentrated in vacuo. The residue was purified by column chromatography on silica gel (eluent: EtOAc/PE=0-30%) to afford rac-methyl (2R,4S)-1-(4-methoxy-2-((1S*,2S*)-2-(4-methylpyrimidin-2-yl)cyclopropyl)quinolin-7-yl)-4-((triisopropylsilyl)oxy)pyrrolidine-2-carboxylate (2.4 g, yield 75%) as a pale-yellow solid. ESI-MS [M+H]+: 591.3

### Synthesis of rac-2-chloro-1-((2R,4S)-1-(4-methoxy-2-((1S*,2S*)-2-(4-methylpyrimidin-2-yl)cyclopropyl)quinolin-7-yl)-4-((triisopropylsilyl)oxy)pyrrolidin-2-yl)ethan-1-one

To a mixture of rac-methyl (2R,4S)-1-(4-methoxy-2-((1S*,2S*)-2-(4-methylpyrimidin-2-yl)cyclopropyl)quinolin-7-yl)-4-((triisopropylsilyl)oxy)pyrrolidine-2-carboxylate (2.4 g, 4.1 mmol) and chloroiodomethane (2.9 g, 16 mmol) in THF (30 mL) was slowly added LDA (8.1 mL, 16.25 mmol, 2M in THF) at −78° C. The reaction was stirred at −78° C. for 30 min. A solution of AcOH (0.98 g, 16 mmol) in THF (0.5 mL) was slowly added below −60° C. After the mixture was stirred at −78° C. for 20 min, the reaction was quenched with NH₄Cl (Sat., aq., 100 mL), and then extracted with EtOAc (100 mL×3). The combined organic phase was washed brine (40 mL), dried over Na₂SO₄, filtered, and concentrated in vacuo. The residue was purified by column chromatography on silica gel (eluent: EtOAc/PE=0-30%) to afford rac-2-chloro-1-((2R,4S)-1-(4-methoxy-2-((1S*,2S*)-2-(4-methylpyrimidin-2-yl)cyclopropyl)quinolin-7-yl)-4-((triisopropylsilyl)oxy)pyrrolidin-2-yl)ethan-1-one (1.3 g, yield 52%) as a pale-yellow solid. ESI-MS [M+H]+: 611.3

### Synthesis of 7-((2R,4S)-2-(8-bromo-6-cyclopropylimidazo[1,2-a]pyridin-2-yl)-4-((triiso-propylsilyl)oxy)pyrrolidin-1-yl)-4-methoxy-2-((1S*,2S*)-2-(4-methylpyrimidin-2-yl)cyclopropyl) quinoline

To a mixture of rac-2-chloro-1-((2R,4S)-1-(4-methoxy-2-((1S*,2S*)-2-(4-methylpyrimidin-2-yl)cyclopropyl)quinolin-7-yl)-4-((triisopropylsilyl)oxy)pyrrolidin-2-yl)ethan-1-one (0.80 g, 1.3 mmol) and 3-bromo-5-cyclopropylpyridin-2-amine (0.40 g, 1.9 mmol) in 1,4-dioxane (20 mL) was added DIPEA (0.50 g, 3.9 mmol). The reaction was stirred at 95° C. for 24 h then was cooled to room temperature and concentrated in vacuo. The residue was quenched with water (40 mL) and then extracted with EtOAc (30 mL×3). The combined organic phase was washed brine (40 mL), dried over Na₂SO₄, filtered, and concentrated in vacuo. The residue was purified by preparative TLC (eluent: DCM/MeOH=17/1) to afford 7-((2R,4S)-2-(8-bromo-6-cyclopropylimidazo[1,2-a]pyridin-2-yl)-4-((triisopropylsilyl)oxy)pyrrolidin-1-yl)-4-methoxy-2-((1S*,2S*)-2-(4-methylpyrimidin-2-yl)cyclopropyl)quinoline (0.12 g, yield 12%) as pale-yellow solid. ESI-MS [M+H]+: 767.3

### Synthesis off-(6-cyclopropyl-2-((2R,4S)-1-(4-methoxy-2-((1S*,2S*)-2-(4-methylpyrimidin-2-yl)cyclopropyl)quinolin-7-yl)-4-((triisopropylsilyl)oxy)pyrrolidin-2-yl)imidazo[1,2-a]pyridin-8-yl)-3-methylimidazolidine-2,4-dione

To a mixture of 7-((2R,4S)-2-(8-bromo-6-cyclopropylimidazo[1,2-a]pyridin-2-yl)-4-((triisopropylsilyl)oxy)pyrrolidin-1-yl)-4-methoxy-2-((1S*,2S*)-2-(4-methylpyrimidin-2-yl)cyclopropyl)quinoline (122 mg, 0.16 mmol) and 3-methylimidazolidine-2,4-dione (182 mg, 1.6 mmol) in 1,4-dioxane (15 mL) was added Pd₂(dba)₃ (21 mg, 0.023 mmol), Xantphos (27 mg, 0.047 mmol) and Cs₂CO₃ (155 mg, 0.47 mmol). The reaction mixture was stirred at 95° C. overnight under N₂. The reaction was quenched with water (30 mL), extracted with EtOAc (30 mL×3). The combined organic layers were washed with brine (25 mL), dried over Na₂SO₄, filtered, and concentrated in vacuo. The residue was purified by preparative TLC (eluent: PE/EA=1/2) to afford 1-(6-cyclopropyl-2-((2R, 4S)-1-(4-methoxy-2-((1S*,2S*)-2-(4-methylpyrimidin-2-yl)cyclopropyl)quinolin-7-yl)-4-((triisopropylsilyl)oxy)pyrrolidin-2-yl)imidazo[1,2-a]pyridin-8-yl)-3-methylimidazolidine-2,4-dione (110 mg, yield 86%) as a pale-yellow solid. ESI-MS [M+H]+: 801.3

### Synthesis of rac-1-(6-cyclopropyl-2-((2R,4S)-4-hydroxy-1-(4-methoxy-2-((1S*,2S*)-2-(4-methylpyrimidin-2-yl)cyclopropyl)quinolin-7-yl)pyrrolidin-2-yl)imidazo[1,2-a]pyridin-8-yl)-3-methylimidazolidine-2,4-dione

To a solution of 1-(6-cyclopropyl-2-((2R,4S)-1-(4-methoxy-2-((1S*,2S*)-2-(4-methyl pyrimidin-2-yl)cyclopropyl)quinolin-7-yl)-4-((triisopropylsilyl)oxy)pyrrolidin-2-yl)imidazo[1,2-a]pyridin-8-yl)-3-methylimidazolidine-2,4-dione (110 mg, 0.14 mmol) in 1,4-dioxane (5 mL) was added HCl (2.5 mL, 4M in 1,4-dioxane). The reaction mixture was stirred at room temperature for 10 h. The mixture was concentrated in vacuo. The mixture was neutralized with NH₃ (2 mL, 7 M in MeOH), and then concentrated in vacuo. The residue was purified by preparative TLC (eluent: DCM/MeOH=15/1) to afford rac-1-(6-cyclopropyl-2-((2R,4S)-4-hydroxy-1-(4-methoxy-2-((1S*,2S*)-2-(4-methylpyrimidin-2-yl)cyclopropyl)quinolin-7-yl)pyrrolidin-2-yl)imidazo[1,2-a]pyridin-8-yl)-3-methylimidazolidine-2,4-dione (67 mg, yield 74%) as a pale-yellow solid.

ESI-MS [M+H]⁺: 645.3. ¹H NMR (400 MHz, DMSO) δ 8.52 (d, J=5.1 Hz, 1H), 8.15-8.14 (m, 1H), 7.77 (d, J=9.1 Hz, 1H), 7.62-7.61 (m, 1H), 7.36-7.34 (m, 1H), 7.17 (d, J=5.1 Hz, 1H), 6.92 (d, J=9.1 Hz, 1H), 6.72-6.70 (m, 2H), 5.20 (d, J=4.6 Hz, 1H), 5.15-5.13 (m, 1H), 4.94-4.93 (m, 2H), 4.61-4.57 (m, 1H), 3.97-3.90 (m, 4H), 3.30-3.26 (m, 1H), 2.97 (s, 3H), 2.76-2.70 (m, 1H), 2.68-2.63 (m, 1H), 2.41 (s, 3H), 2.35-2.28 (m, 2H), 1.93-1.86 (m, 1H), 1.83-1.75 (m, 1H), 1.72-1.67 (m, 1H), 0.93-0.87 (m, 2H), 0.63-0.58 (m, 2H).

### Example 68

### Synthesis of rac-1-(6-cyclopropyl-2-((2R,4S)-4-hydroxy-1-(2-((1S*,2S*)-2-(4-methylpyrimidin-2-yl)cyclopropyl)quinolin-7-yl)pyrrolidin-2-yl)imidazo[1,2-a]pyridin-8-yl)-3-methylimidazolidine-2,4-dione (I-68)

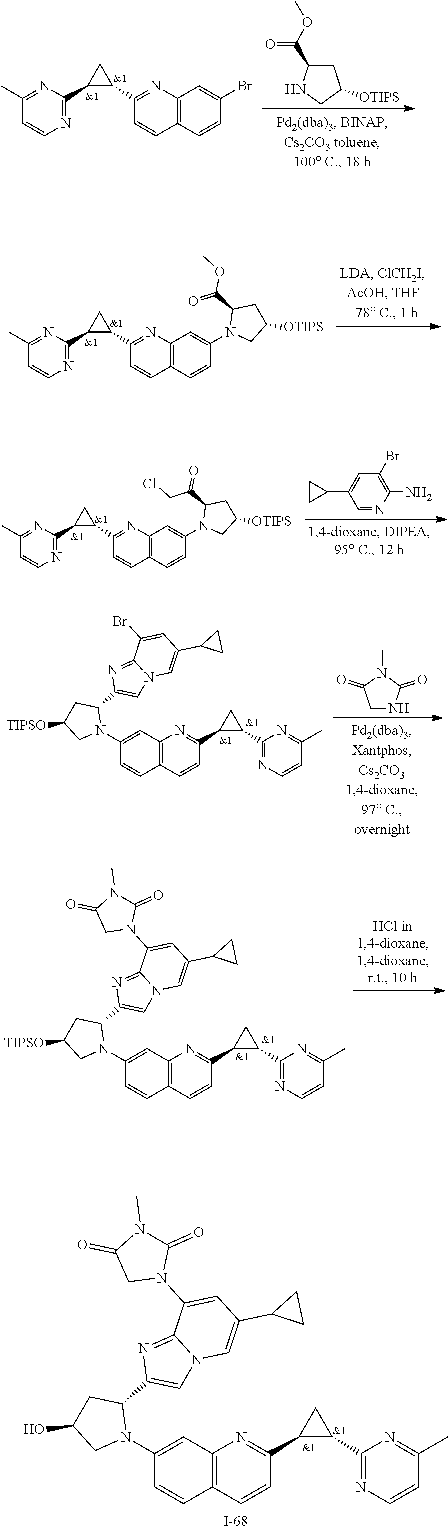

### Synthesis of methyl (2R,4S)-1-(2-((1S*,2S*)-2-(4-methylpyrimidin-2-yl)cyclopropyl)quinolin-7-yl)-4-((triisopropylsilyl)oxy)pyrrolidine-2-carboxylate

To a mixture of rac-7-bromo-2-((1S*,2S*)-2-(4-methylpyrimidin-2-yl)cyclopropyl)quinoline (1.4 g, 4.1 mmol) and methyl (2R,4S)-4-((triisopropylsilyl)oxy)pyrrolidine-2-carboxylate (2.0 g, 6.6 mmol) in toluene (40 mL) was added Pd₂(dba)₃ (375 mg, 0.41 mmol), BINAP (510 mg, 0.82 mmol) and Cs₂CO₃ (4.0 g, 12.2 mmol). The mixture was stirred at 100° C. for 18 h under N₂. The reaction was cooled to room temperature, filtered and concentrated in vacuo. The residue was diluted with water (50 mL), extracted with EtOAc (40 mL×3). The combined organic layers were washed brine (40 mL), dried over Na₂SO₄, filtered, and concentrated in vacuo. The residue was purified by column chromatography on silica gel (eluent: EtOAc/PE=0-50%) to afford methyl (2R,4S)-1-(2-((1S*,2S*)-2-(4-methylpyrimidin-2-yl)cyclopropyl)quinolin-7-yl)-4-((triisopropylsilyl)oxy)pyrrolidine-2-carboxylate (1.6 g, yield 70%) as a pale-yellow solid. ESI-MS [M+H]+: 561.1

### Synthesis of 2-chloro-7-((2R,4S)-1-(2-((1S*,2S*)-2-(4-methylpyrimidin-2-yl)cyclopropyl)quinolin-7-yl)-4-((triisopropylsilyl)oxy)pyrrolidin-2-yl)ethan-1-one

To a mixture of methyl (2R, 4S)-1-(2-((1S*,2S*)-2-(4-methylpyrimidin-2-yl)cyclopropyl)quinolin-7-yl)-4-((triisopropylsilyl)oxy)pyrrolidine-2-carboxylate (1.6 g, 2.85 mmol) and chloroiodomethane (2.0 g, 11 mmol) in THF (30 mL) was slowly added lithium diisopropylamide (5.5 mL, 11 mmol, 2 M in THF) at −78° C. The reaction was stirred at −78° C. for 30 min. Then a solution of acetic acid (660 mg, 11 mmol) in THF (0.5 mL) was slowly added below −60° C. The resulting mixture was stirred at −78° C. for 20 min. Then the reaction was quenched with NH₄Cl (Sat. aq., 100 mL) and then extracted with EtOAc (100 mL×3). The combined organic layers were washed brine (40 mL), dried over Na₂SO₄, filtered and concentrated in vacuo. The residue was purified by column chromatography on silica gel (eluent: EtOAc/PE=0-26%) to afford 2-chloro-1-((2R,4S)-1-(2-((1S*,2S*)-2-(4-methylpyrimidin-2-yl)cyclopropyl)quinolin-7-yl)-4-((triisopropylsilyl)oxy)pyrrolidin-2-yl)ethan-1-one (810 mg, yield 49%) as pale yellow sticky oil.

ESI-MS [M+H]+: 579.1

### Synthesis of 7-((2R,4S)-2-(8-bromo-6-cyclopropylimidazo[1,2-a]pyridin-2-yl)-4-((triiso-propylsilyl)oxy)pyrrolidin-1-yl)-2-((1S*,2S*)-2-(4-methylpyrimidin-2-yl)cyclopropyl)quinoline

To a mixture of 2-chloro-1-((2R,4S)-1-(2-((1S*,2S*)-2-(4-methylpyrimidin-2-yl)cyclopropyl)quinolin-7-yl)-4-((triisopropylsilyl)oxy)pyrrolidin-2-yl)ethan-1-one (810 mg, 1.4 mmol) and 3-bromo-5-cyclopropylpyridin-2-amine (596 mg, 2.8 mmol) in 1,4-dioxane (50 mL) was added DIPEA (722 mg, 5.6 mmol). The reaction was stirred at 95° C. for 12 h. The reaction was cooled to room temperature and concentrated in vacuo. The residue was diluted with water (30 mL) and then extracted with EtOAc (30 mL×3). The combined organic phase was washed brine (40 mL), dried over Na₂SO₄, filtered and concentrated in vacuo. The residue was purified by preparative TLC (eluent: DCM/MeOH=20/1) to afford 7-((2R,4S)-2-(8-bromo-6-cyclopropylimidazo[1,2-a]pyridin-2-yl)-4-((triisopropylsilyl)oxy)pyrrolidin-1-yl)-2-((1S*,2S*)-2-(4-methylpyrimidin-2-yl)cyclopropyl)quinoline (170 mg, 17%) as a brown solid. ESI-MS [M+H]+: 737.1

### Synthesis of 1-(6-cyclopropyl-2-((2R,4S)-1-(2-((1S*,2S*)-2-(4-methylpyrimidin-2-yl)cyclopropyl) quinolin-7-yl)-4-((triisopropylsilyl)oxy)pyrrolidin-2-yl)imidazo[1,2-a]pyridin-8-yl)-3-methylimidazolidine-2,4-dione

To a mixture of 7-((2R,4S)-2-(8-bromo-6-cyclopropylimidazo[1,2-a]pyridin-2-yl)-4-((triisopropylsilyl)oxy)pyrrolidin-1-yl)-2-((1S*,2S*)-2-(4-methylpyrimidin-2-yl)cyclopropyl)quinoline (120 mg, 0.16 mmol) and 3-methylimidazolidine-2,4-dione (194 mg, 1.7 mmol) in 1,4-dioxane (15 mL) was added Pd₂(dba)₃ (26 mg, 0.024 mmol), Xantphos (28 mg, 0.049 mmol) and Cs₂CO₃ (160 mg, 0.49 mmol). The mixture was stirred at 97° C. overnight. The mixture was cooled to room temperature, filtered and concentrated in vacuo. The residue was diluted with water (20 mL), and then extracted with EtOAc (25 mL×3). The combined organic layers were washed with brine (30 mL), dried over Na₂SO₄, filtered and concentrated in vacuo. The residue was purified by preparative TLC (eluent: PE/EtOAc=2:1) to 1-(6-cyclopropyl-2-((2R, 4S)-1-(2-((1S*,2S*)-2-(4-methylpyrimidin-2-yl)cyclopropyl)quinolin-7-yl)-4-((triisopropylsilyl)oxy)pyrrolidin-2-yl)imidazo[1,2-a]pyridin-8-yl)-3-methylimidazolidine-2,4-dione (74 mg, 60%) as a pale-yellow solid. ESI-MS [M+H]+: 771.3

### Synthesis of rac-1-(6-cyclopropyl-2-((2R,4S)-4-hydroxy-1-(2-((1S*,2S*)-2-(4-methylpyrimidin-2-yl)cyclopropyl)quinolin-7-yl)pyrrolidin-2-yl)imidazo[1,2-a]pyridin-8-yl)-3-methylimidazolidine-2,4-dione

To a mixture of 1-(6-cyclopropyl-2-((2R, 4S)-1-(2-((1S*,2S*)-2-(4-methylpyrimidin-2-yl)cyclopropyl)quinolin-7-yl)-4-((triisopropylsilyl)oxy)pyrrolidin-2-yl)imidazo[1,2-a]pyridin-8-yl)-3-methylimidazolidine-2,4-dione (74 mg, 0.096 mmol) in 1,4-dioxane (4 mL) was added HCl (2.0 mL, 4M in 1,4-dioxane). The reaction was stirred at room temperature for 10 h. The reaction was concentrated in vacuo. The residue was neutralized with NH₃ (7 M in MeOH, 2.0 mL) then concentrated in vacuo and purified by preparative TLC (eluent: DCM/MeOH=10/1) to afford 1-(6-cyclopropyl-2-((2R, 4S)-4-hydroxy-1-(2-((1S*,2S*)-2-(4-methylpyrimidin-2-yl)cyclopropyl)quinolin-7-yl)pyrrolidin-2-yl)imidazo[1,2-a]pyridin-8-yl)-3-methylimidazolidine-2,4-dione (55 mg, 93%) as a pale-yellow solid.

ESI-MS [M+H]+: 615.3. ¹H NMR (400 MHz, DMSO) δ 8.51 (d, J=5.1 Hz, 1H), 8.15-8.14 (m, 1H), 7.92 (d, J=8.3 Hz, 1H), 7.63 (d, J=3.4 Hz, 1H), 7.61-7.58 (m, 1H), 7.36-7.34 (m, 1H), 7.17 (d, J=5.1 Hz, 1H), 7.12 (d, J=8.3 Hz, 1H), 7.01-6.98 (m, 1H), 6.77-6.75 (m, 1H), 5.21-5.15 (m, 2H), 4.95-4.94 (m, 2H), 4.61-4.58 (m, 1H), 3.96-3.92 (m, 1H), 3.30-3.28 (m, 1H), 2.97 (s, 3H), 2.71-2.65 (m, 2H), 2.41 (s, 3H), 2.36-2.28 (m, 2H), 1.93-1.87 (m, 1H), 1.78-1.69 (m, 2H), 0.93-0.87 (m, 2H), 0.63-0.57 (m, 2H).

### Example 69

### Synthesis of rac-1-(6-cyclopropyl-2-(((4-(2-hydroxyethoxy)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (I-69)

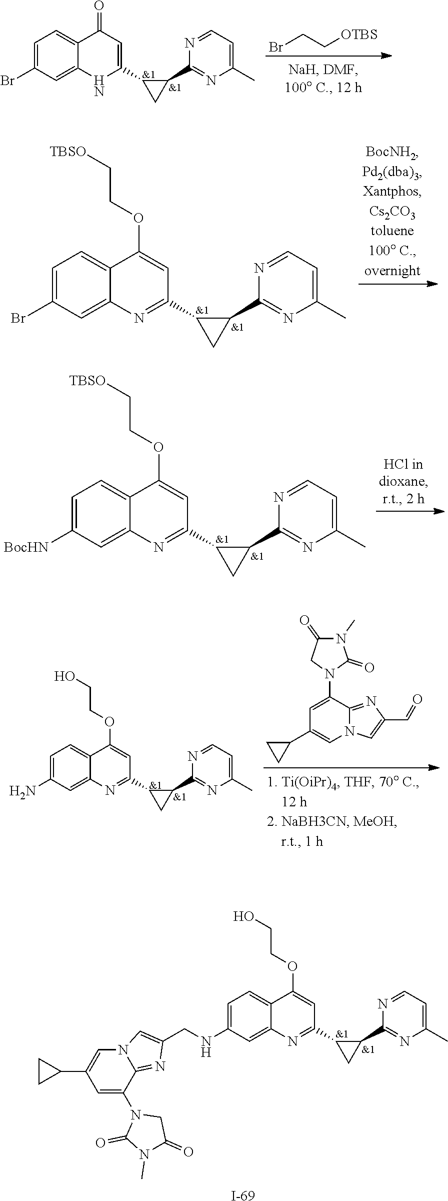

### Synthesis of rac-7-bromo-4-(2-((tert-butyldimethylsilyl)oxy)ethoxy)-2-((1S*2S*)-2-(4-methylpyrimidin-2-yl)cyclopropyl)quinoline

A mixture of rac-7-bromo-2-((1S*,2S*)-2-(4-methylpyrimidin-2-yl)cyclopropyl)quinolin-4 (1H)-one (285 mg, 0.80 mmol), (2-bromoethoxy)(tert-butyl)dimethylsilane (382 mg, 1.60 mmol) and NaH (128 mg, 3.20 mmol, 60% dispersion in mineral oil) in DMF (8 mL) was stirred at 100° C. for 12 h. The reaction was cooled to room temperature and quenched with NH₄Cl (sat. aq., 50 mL), extracted with EtOAc (40 mL×3). The combined organic layers were washed with brine (30 mL), dried over Na₂SO₄, filtered and concentrated in vacuo to give the crude, which was purified by preparative TLC (eluent: PE/EtOAc=1/1) to give rac-7-bromo-4-(2-((tert-butyldimethylsilyl)oxy)ethoxy)-2-((1S*,2S*)-2-(4-methylpyrimidin-2-yl)cyclopropyl)quinoline (210 mg, 51%) as a yellow solid.

ESI-MS [M+H]+: 514.2

### Synthesis of rac-tert-butyl (4-(2-((tert-butyldimethylsilyl)oxy)ethoxy)-2-((1S*,2S*)-2-(4-methyl pyrimidin-2-yl)cyclopropyl)quinolin-7-yl)carbamate

A mixture of rac-7-bromo-4-(2-((tert-butyldimethylsilyl)oxy)ethoxy)-2-((1S*,2S*)-2-(4-methylpyrimidin-2-yl)cyclopropyl)quinoline (210 mg, 0.41 mmol), tert-butyl carbamate (96 mg, 0.82 mmol), Pd₂(dba)₃ (57 mg, 0.062 mmol), Xantphos (69 mg, 0.12 mmol) and Cs₂CO₃ (358 mg, 1.1 mmol) in toluene was stirred at 110° C. overnight under N₂. The reaction mixture was filtered, filter cake was washed with DCM/MeOH (10/1, 50 mL). The filtrate was concentrated in vacuo to give the crude, which was purified with preparative TLC (eluent: PE/EtOAc=) to give rac-tert-butyl (4-(2-((tert-butyldimethylsilyl)oxy)ethoxy)-2-((1S*,2S*)-2-(4-methylpyrimidin-2-yl)cyclopropyl)quinolin-7-yl)carbamate (150 mg, 66%) as a yellow solid.

ESI-MS [M+H]+: 551.1.

### Synthesis of rac-2-((7-amino-2-((1R,2R)-2-(4-methylpyrimidin-2-yl)cyclopropyl)quinolin-4-yl)oxy)ethan-1-ol

To a solution of rac-tert-butyl (4-(2-((tert-butyldimethylsilyl)oxy)ethoxy)-2-((1S*,2S*)-2-(4-methylpyrimidin-2-yl)cyclopropyl)quinolin-7-yl)carbamate (150 mg, 0.27 mmol) in dioxane (4 mL) was added HCl (1 mL, 4M solution in dioxane). The resulting reaction was stirred at room temperature for 2 h. The reaction was concentrated to give rac-2-((7-amino-2-((1R,2R)-2-(4-methylpyrimidin-2-yl)cyclopropyl)quinolin-4-yl)oxy)ethan-1-ol (140 mg, crude) as yellow oil, which was used into the next step without further purification. ESI-MS [M+H]+: 337.1.

### Synthesis of rac-1-(6-cyclopropyl-2-(((4-(2-hydroxyethoxy)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione

A mixture of rac-2-((7-amino-2-((1R,2R)-2-(4-methylpyrimidin-2-yl)cyclopropyl)quinolin-4-yl)oxy)ethan-1-ol (140 mg, crude), 6-cyclopropyl-8-(3-methyl-2,4-dioxoimidazolidin-1-yl)imidazo[1,2-a]pyridine-2-carbaldehyde (72 mg, 0.24 mmol) and Ti(OiPr)₄ (340 mg, 1.20 mmol) in THF (10 mL) was stirred at 70° C. for 12 h. The reaction was cooled to room temperature, MeOH (2 mL) was added, followed by addition of NaBH₃CN (60 mg, 0.96 mmol). The resulting reaction mixture was stirred at room temperature for 1 h then quenched with water (30 mL) and extracted with EtOAc (40 mL×3). The combined organic layers were washed with brine (30 mL), dried over Na₂SO₄, filtered and concentrated in vacuo to give the crude, which was purified by preparative TLC (eluent: DCM/MeOH=20/1) to give rac-1-(6-cyclopropyl-2-(((4-(2-hydroxyethoxy)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (74 mg, 50%) as a white solid.

ESI-MS [M+H]+: 619.3, ¹H NMR (400 MHz, DMSO) δ 8.51 (d, J=5.1 Hz, 1H), 8.25 (s, 1H), 7.84 (d, J=9.0 Hz, 1H), 7.75 (s, 1H), 7.35 (s, 1H), 7.17 (d, J=5.1 Hz, 1H), 6.97 (d, J=8.1 Hz, 1H), 6.74-6.70 (m, 3H), 5.03-4.89 (m, 3H), 4.47 (d, J=5.5 Hz, 2H), 4.17 (s, 2H), 3.83-3.77 (m, 2H), 2.98 (s, 3H), 2.71-2.60 (m, 2H), 2.41 (s, 3H), 1.95-1.88 (m, 1H), 1.72-1.66 (m, 2H), 0.98-0.86 (m, 2H), 0.68-0.57 (m, 2H).

### Example 70

### Synthesis of rac-1-(6-cyclopropyl-2-(((2-((1S*,2S*)-2-(4-methylpyrimidin-2-yl)cyclopropyl) quinolin-8-yl)amino)methyl)imidazo[1,2-a]pyridin-8-yl)-3-methylimidazolidine-2,4-dione (I-70)

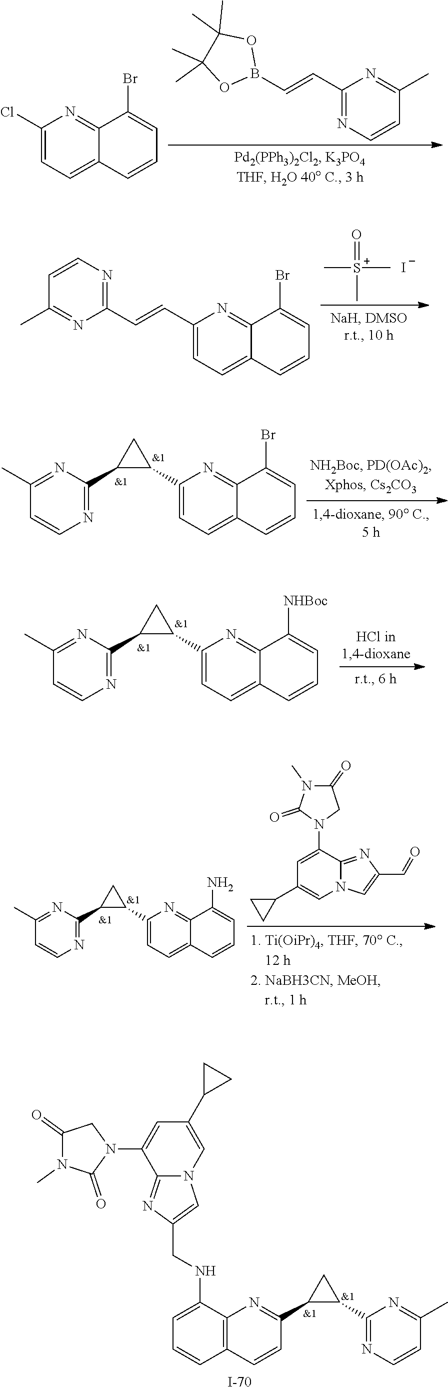

### Synthesis of (E)-8-bromo-2-(2-(4-methylpyrimidin-2-yl)vinyl)quinoline

A mixture of 8-bromo-2-chloroquinoline (484 mg, 2.0 mmol), (E)-4-methyl-2-(2-(4,4,5,5-tetramethyl-1,3,2-dioxaborolan-2-yl)vinyl)pyrimidine (492 mg, 2.0 mmol), Pd(PPh₃)₂Cl₂ (145 mg, 0.20 mmol) and K₃PO₄ (1.3 g, 6.0 mmol) in THF/H₂O (10/1, 11 mL) was stirred at 40° C. for 3 h under N₂. The reaction mixture was concentrated in vacuo to give a residue, which was purified by column chromatography on silica gel, eluting with 0˜10% MeOH/DCM to give (E)-8-bromo-2-(2-(4-methyl pyrimidin-2-yl)vinyl)quinoline (300 mg, 46%) as a light yellow solid. ESI-MS [M+H]⁺: 326.0.

### Synthesis of rac-8-bromo-2-((1S*,2S*)-2-(4-methylpyrimidin-2-yl)cyclopropyl)quinoline

To a stirred solution of NaH (100 mg, 60% in mineral oil, 2.5 mmol) in DMSO (5 mL) was added trimethylsulfoxonium iodide (552 mg, 2.5 mmo) at 0° C. The resulting mixture was stirred at room temperature for 1 h under N₂. A solution of (E)-8-bromo-2-(2-(4-methylpyrimidin-2-yl)vinyl) quinoline (260 mg, 0.79 mmol) in DMSO (2.0 mL) was added and the resulting mixture was stirred at room temperature for 10 h. Then the reaction was quenched with water (30 mL) and extracted with EtOAc (50 mL×3). The combined organic layers were washed with brine (50 mL), dried over Na₂SO₄, and then concentrated in vacuo to give the crude, which was purified by preparative TLC (eluting with 0˜5% MeOH/DCM to give rac-8-bromo-2-((1S*,2S*)-2-(4-methylpyrimidin-2-yl)cyclopropyl)quinoline (100 mg, 37%) as a light yellow solid. ESI-MS [M+H]+: 340.0.

### Synthesis of rac-tert-butyl (2-((1S*,2S*)-2-(4-methylpyrimidin-2-yl)cyclopropyl)quinolin-8-yl) carbamate

A mixture of rac-8-bromo-2-((1S*,2S*)-2-(4-methylpyrimidin-2-yl)cyclopropyl)quinoline (100 mg, 0.29 mmol), NH₂Boc (68 mg, 0.58 mmol), Pd(OAc)₂ (7.0 mg, 0.029 mmol), X-Phos (14 mg, 0.029 mmol) and Cs₂CO₃ (280 mg, 0.86 mmol) in 1,4-dioxane (10 mL) was stirred at 90° C. for 5 h under N₂. The mixture was filtered through the Celite® and washed with DMC/MeOH (10/1, 20 mL). The filtrate was concentrated in vacuo to give a crude, which was purified by preparative TLC, eluting with 5% MeOH/DCM to give rac-tert-butyl (2-((1S*,2S*)-2-(4-methylpyrimidin-2-yl)cyclopropyl) quinolin-8-yl)carbamate (67 mg, 61%) as a light yellow solid. ESI-MS [M+H]⁺: 377.2.

### Synthesis of rac-2-((1S*,2S*)-2-(4-methylpyrimidin-2-yl)cyclopropyl)quinolin-8-amine

A solution of rac-tert-butyl (2-((1S*,2S*)-2-(4-methylpyrimidin-2-yl)cyclopropyl)quinolin-8-yl) carbamate (80 mg, 0.21 mmol) in 1,4-dioxane (3.0 mL) and HCl (4 N in 1,4-dioxane, 3.0 mL, 12 mmol) was stirred at room temperature for 6 h. The mixture was concentrated in vacuo to get a residue, which was diluted with NaHCO₃ (sat. aq., 20 mL) and extracted with EtOAc (20 mL×3). The combined organic layers were washed with brine (20 mL), dried over Na₂SO₄ then concentrated in vacuo to give the crude, which was purified by preparative TLC, eluting with 10% MeOH/DCM to give rac-2-((1S*,2S*)-2-(4-methylpyrimidin-2-yl)cyclopropyl)quinolin-8-amine (30 mg, 52%) as a light yellow solid. ESI-MS [M+H]⁺: 277.1.

### Synthesis of rac-1-(6-cyclopropyl-2-(((2-((1S*,2S*)-2-(4-methylpyrimidin-2-yl)cyclopropyl)quinolin-8-yl)amino)methyl)imidazo[1,2-a]pyridin-8-yl)-3-methylimidazolidine-2,4-dione

A mixture of rac-2-((1S*,2S*)-2-(4-methylpyrimidin-2-yl)cyclopropyl)quinolin-8-amine (38 mg, 0.13 mmol), 6-cyclopropyl-8-(3-methyl-2,4-dioxoimidazolidin-1-yl)imidazo[1,2-a]pyridine-2-carbaldehyde (36 mg, 0.13 mmol) and Ti(OiPr)₄ (170 mg, 0.60 mmol) in THF (5 mL) was stirred for 10 h at 70° C. under N₂. After cooling to room temperature, the mixture was added MeOH (1 mL) and NaBH₃CN (28 mg, 0.45 mmol) subsequently. The resulting mixture was stirred at room temperature for 1 h at room temperature. The reaction was quenched with H₂O (20 mL) and extracted with EtOAc (20 mL×3). The combined organic layers were washed with brine (20 mL), dried over Na₂SO₄, then concentrated in vacuo to give the crude, which was purified by preparative TLC (eluting with 6% MeOH/DCM) to give rac-1-(6-cyclopropyl-2-(((2-((1S*,2S*)-2-(4-methylpyrimidin-2-yl)cyclopropyl)quinolin-8-yl)amino)methyl)imidazo[1,2-a]pyridin-8-yl)-3-methylimidazolidine-2,4-dione (9.4 mg, 13%) as a light yellow solid.

ESI-MS [M+H]⁺: 559.2, ¹H NMR (400 MHz, DMSO) δ 8.52 (d, J=5.1 Hz, 1H), 8.24 (s, 1H), 8.10 (d, J=8.5 Hz, 1H), 7.76 (s, 1H), 7.54 (d, J=8.4 Hz, 1H), 7.34 (d, J=1.3 Hz, 1H), 7.24 (t, J=7.9 Hz, 1H), 7.18 (d, J=5.1 Hz, 1H), 7.08-7.02 (m, 2H), 6.67 (d, J=7.7 Hz, 1H), 5.02-4.88 (m, 2H), 4.62 (d, J=5.8 Hz, 2H), 2.91 (s, 3H), 2.89-2.80 (m, 2H), 2.42 (s, 3H), 1.96-1.89 (m, 2H), 1.83-1.79 (m, 1H), 0.95-0.90 (m, 2H), 0.65-0.61 (m, 2H).

### Examples 71-72

### Synthesis of rac-1-(6-cyclopropyl-2-((5-((1S*,2S*)-2-(4-methylpyrimidin-2-yl)cyclopropyl)-3H-imidazo[4,5-b]pyridin-3-yl)methyl)imidazo[1,2-a]pyridin-8-yl)-3-methylimidazolidine-2,4-dione (I-71) and rac-1-(6-cyclopropyl-2-((5-((1S*,2S*)-2-(4-methylpyrimidin-2-yl)cyclopropyl)-1H-imidazo[4,5-b]pyridin-1-yl)methyl)imidazo[1,2-a]pyridin-8-yl)-3-methylimidazolidine-2,4-dione (I-72)

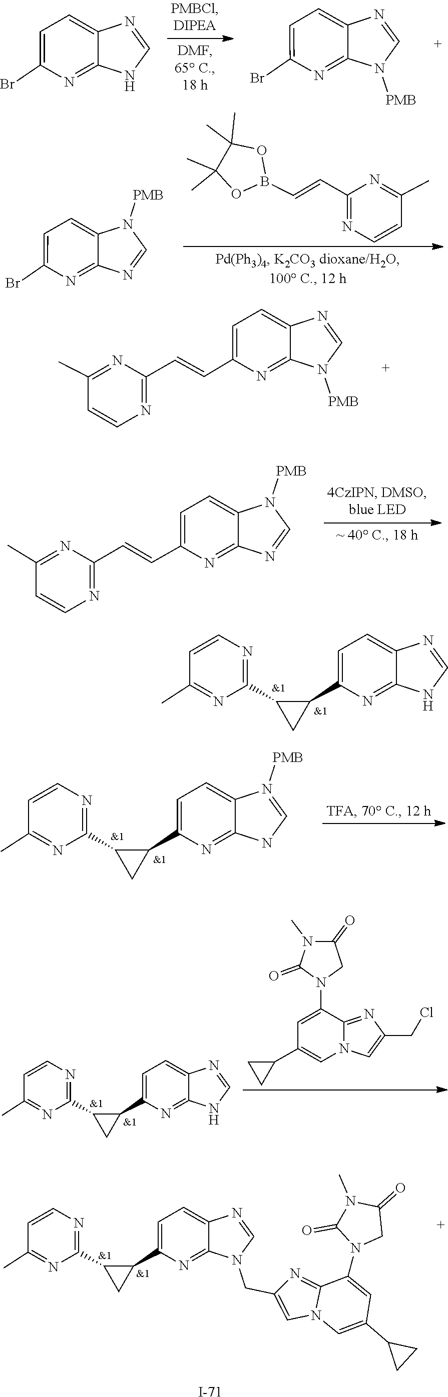

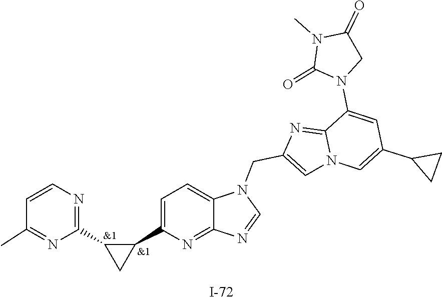

### Synthesis of 5-bromo-3-(4-methoxybenzyl)-3H-imidazo[4,5-b]pyridine and 5-bromo-1-(4-methoxybenzyl)-1H-imidazo[4,5-b]pyridine

To a solution of 5-bromo-3H-imidazo[4,5-b]pyridine (4.0 g, 20.4 mmol) in DMF (50 mL) was added K₂CO₃ (5.6 g, 40.8 mmol), PMBCl (4.8 g, 30.6 mmol) and the mixture was stirred at 65° C. for 18 h. The mixture was quenched with NaHCO₃ (sat. aq., 100 mL) and extracted with EtOAc (60 mL×3). The combined organic layers were washed with brine (60 mL), dried over Na₂SO₄ and concentrated in vacuo. The residue was purified by column chromatography (eluent: EtOAc/PE from 0 to 30%) to give 5-bromo-3-(4-methoxybenzyl)-3H-imidazo[4,5-b]pyridine and 5-bromo-1-(4-methoxybenzyl)-1H-imidazo[4,5-b]pyridine (3.1 g as a mixture, yield 48%) as a yellow solid. ESI-MS [M+H]⁺: 318.0

### Synthesis of (E)-3-(4-methoxybenzyl)-5-(2-(4-methylpyrimidin-2-yl)vinyl)-3H-imidazo[4,5-b]pyridine and of (E)-1-(4-methoxybenzyl)-5-(2-(4-methylpyrimidin-2-yl)vinyl)-3H-imidazo[4,5-b]pyridine

To a solution of 5-bromo-3-(4-methoxybenzyl)-3H-imidazo[4,5-b]pyridine and 5-bromo-1-(4-methoxybenzyl)-3H-imidazo[4,5-b]pyridine (2.5 g, 7.9 mmol) in 1,4-dioxane/H₂O (50 mL/10 mL) was added (E)-4-methyl-2-(2-(4,4,5,5-tetramethyl-1,3,2-dioxaborolan-2-yl)vinyl)pyrimidine (2.5 g, 10.2 mmol), K₂CO₃ (3.3 g, 23.6 mmol) and Pd(PPh₃)₄ (1.4 g, 1.2 mmol). The mixture was stirred at 100° C. for 12 h under N₂. The reaction mixture was quenched with water (50 mL) and extracted with EtOAc (50 mL×3). The combined organic layers were washed with brine (50 mL), dried over Na₂SO₄ and concentrated in vacuo. The residue was purified by column chromatography (eluent: MeOH:DCM=0˜5%) to give (E)-3-(4-methoxybenzyl)-5-(2-(4-methylpyrimidin-2-yl)vinyl)-3H-imidazo[4,5-b]pyridine and (E)-1-(4-methoxybenzyl)-5-(2-(4-methylpyrimidin-2-yl)vinyl)-3H-imidazo[4,5-b]pyridine (2.3 g as a mixture, yield 82%) as a yellow solid. ESI-MS [M+H]⁺: 358.2

### Synthesis of rac-3-(4-methoxybenzyl)-5-((1S*,2S*)-2-(4-methylpyrimidin-2-yl)cyclopropyl)-3H-imidazo[4,5-b]pyridine and rac-n-(4-methoxybenzyl)-5-((1S*,2S*)-2-(4-methylpyrimidin-2-yl)cyclopropyl)-3H-imidazo[4,5-b]pyridine

A mixture of (E)-3-(4-methoxybenzyl)-5-(2-(4-methylpyrimidin-2-yl)vinyl)-3H-imidazo[4,5-b]pyridine and (E)-1-(4-methoxybenzyl)-5-(2-(4-methylpyrimidin-2-yl)vinyl)-3H-imidazo[4,5-b]pyridine (100 mg as a mixture, 0.28 mmol), triethylammonium bis(catecholato)iodomethylsilicate (205 mg, 0.42 mmol), 4CzIPN (32 mg, 0.04 mmol) in DMSO (6 mL) was degassed under N₂ for 3 times. Then the mixture was placed in front of one blue LED and stirred at 40° C. for 18 h. The reaction mixture was quenched by ice water (20 mL) and extracted with EtOAc (20 mL×3). The combined organic layers were washed with brine (20 mL), dried over Na₂SO₄ and concentrated in vacuo. The residue was purified by preparative TLC (eluted: DCM:MeOH=15:1) to give rac-3-(4-methoxybenzyl)-5-((1S*,2S*)-2-(4-methylpyrimidin-2-yl)cyclopropyl)-3H-imidazo[4,5-b]pyridine and rac-1-(4-methoxybenzyl)-5-((1S*,2S*)-2-(4-methylpyrimidin-2-yl)cyclopropyl)-3H-imidazo[4,5-b]pyridine (84 mg as a mixture, yield 81%) as a yellow solid. ESI-MS [M+H]⁺: 372.2

### Synthesis of rac-5-((1S*,2S*)-2-(4-methylpyrimidin-2-yl)cyclopropyl)-3H-imidazo[4,5-b]pyridine

A mixture of rac-3-(4-methoxybenzyl)-5-((1S*,2S*)-2-(4-methylpyrimidin-2-yl)cyclopropyl)-3H-imidazo[4,5-b]pyridine and rac-1-(4-methoxybenzyl)-5-((1S*,2S*)-2-(4-methylpyrimidin-2-yl)cyclopropyl)-3H-imidazo[4,5-b]pyridine (84 mg as mixture, 0.23 mmol) in TFA (3 mL) was stirred at 70° C. for 12 h. The reaction mixture was quenched with NaHCO₃ (sat. aq., 100 mL) and extracted with extracted with EtOAc (30 mL×3). The combined organic layers were washed with brine (30 mL), dried over Na₂SO₄ and concentrated in vacuo. The residue was purified by preparative TLC (eluted: DCM:MeOH=20:1) to give rac-5-((1S*,2S*)-2-(4-methylpyrimidin-2-yl)cyclopropyl)-3H-imidazo[4,5-b]pyridine (15 mg, yield 26%). ESI-MS [M+H]⁺: 252.2

### Synthesis of rac-1-(6-cyclopropyl-2-((5-((1S*,2S*)-2-(4-methylpyrimidin-2-yl)cyclopropyl)-3H-imidazo[4,5-b]pyridin-3-yl)methyl)imidazo[1,2-a]pyridin-8-yl)-3-methylimidazolidine-2,4-dione and rac-1-(6-cyclopropyl-2-((5-((1S*,2S*)-2-(4-methylpyrimidin-2-yl)cyclopropyl)-1H-imidazo[4,5-b]pyridin-1-yl)methyl)imidazo[1,2-a]pyridin-8-yl)-3-methylimidazolidine-2,4-dione

To a mixture of 1-(2-(chloromethyl)-6-cyclopropylimidazo[1,2-a]pyridin-8-yl)-3-methylimidazolidine-2,4-dione (29 mg, 0.09 mmol) and K₂CO₃ (41 mg, 0.30 mmol) in DMF (1 mL) was added 3-(4-methoxybenzyl)-5-(2-(4-methylpyrimidin-2-yl)cyclopropyl)-3H-imidazo[4,5-b]pyridine (15 mg, 0.06 mmol) in DMF (2 mL). The mixture was stirred at 70° C. for 12 h. The reaction mixture was quenched with water (30 mL) and extracted with EtOAc (30 mL×3). The combined organic layers were washed with brine (30 mL), dried over Na₂SO₄ and concentrated in vacuo. The residue was purified by preparative TLC (eluted: DCM:MeOH=20:1) to give rac-1-(6-cyclopropyl-2-((5-((1S*,2S*)-2-(4-methylpyrimidin-2-yl)cyclopropyl)-3H-imidazo[4,5-b]pyridin-3-yl)methyl) imidazo [1,2-a]pyridin-8-yl)-3-methylimidazolidine-2,4-dione (12 mg, yield 38%) and rac-1-(6-cyclopropyl-2-((5-((1S*,2S*)-2-(4-methylpyrimidin-2-yl)cyclopropyl)-1H-imidazo[4,5-b]pyridin-1-yl)methyl) imidazo[1,2-a]pyridin-8-yl)-3-methylimidazolidine-2,4-dione (6 mg, yield 19%) as white solid.

rac-1-(6-cyclopropyl-2-((5-((1S*,2S*)-2-(4-methylpyrimidin-2-yl)cyclopropyl)-3H-imidazo[4,5-b]pyridin-3-yl)methyl)imidazo[1,2-a]pyridin-8-yl)-3-methylimidazolidine-2,4-dione (Example 71, I-71)

ESI-MS [M+H]⁺: 534.2. ¹H NMR (400 MHz, DMSO) δ 8.54-8.47 (m, 2H), 8.28-8.23 (m, 1H), 7.95 (d, J=8.2 Hz, 1H), 7.79 (s, 1H), 7.39-7.36 (m, 1H), 7.30 (d, J=8.2 Hz, 1H), 7.17 (d, J=5.1 Hz, 1H), 5.55 (s, 2H), 4.87 (s, 2H), 2.96 (s, 3H), 2.77-2.73 (m, 1H), 2.71-2.67 (m, 1H), 2.41 (s, 3H), 1.98-1.90 (m, 1H), 1.80-1.71 (m, 2H), 0.96-0.91 (m, 2H), 0.65-0.60 (m, 2H).

rac-1-(6-cyclopropyl-2-((5-((1S*,2S*)-2-(4-methylpyrimidin-2-yl)cyclopropyl)-1H-imidazo[4,5-b]pyridin-1-yl)methyl)imidazo[1,2-a]pyridin-8-yl)-3-methylimidazolidine-2,4-dione (Example 72, I-72)

ESI-MS [M+H]⁺: 534.2. ¹H NMR (400 MHz, DMSO) δ 8.56 (s, 1H), 8.51 (d, J=5.1 Hz, 1H), 8.26-8.21 (m, 1H), 7.99 (d, J=8.3 Hz, 1H), 7.81 (s, 1H), 7.39-7.36 (m, 1H), 7.28 (d, J=8.3 Hz, 1H), 7.16 (d, J=5.1 Hz, 1H), 5.59 (s, 2H), 4.83 (s, 2H), 2.95 (s, 3H), 2.73-2.68 (m, 1H), 2.67-2.63 (m, 1H), 2.41 (s, 3H), 1.97-1.88 (m, 1H), 1.75-1.67 (m, 2H), 0.95-0.90 (m, 2H), 0.64-0.60 (m, 2H).

### Example 73

### Synthesis of rac-7-(((6-cyclopropyl-8-(3-methyl-2,4-dioxoimidazolidin-1-yl)imidazo[1,2-a]pyridin-2-yl)methyl)amino)-2-((1S*,2S*)-2-(4-methylpyrimidin-2-yl)cyclopropyl)quinoline-4-carbonitrile (I-73)

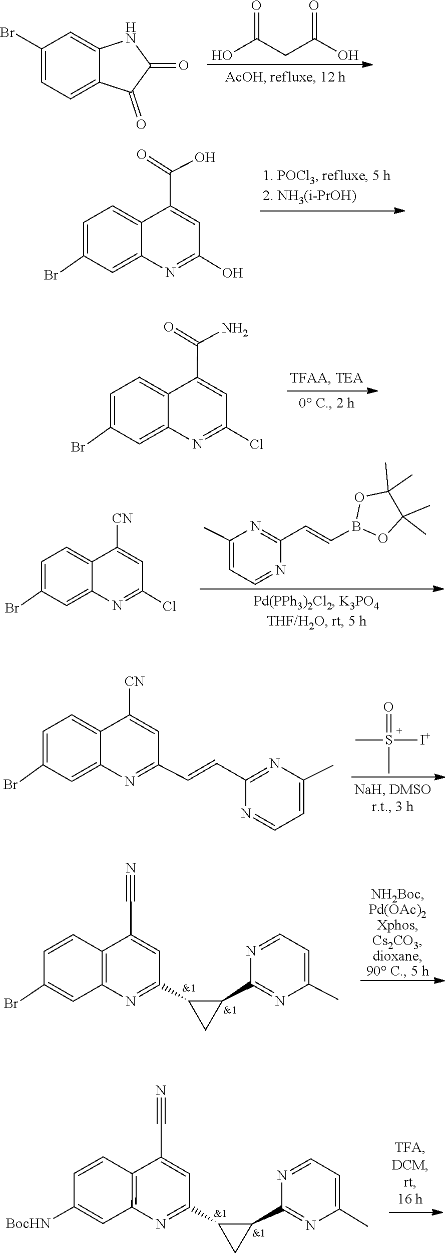

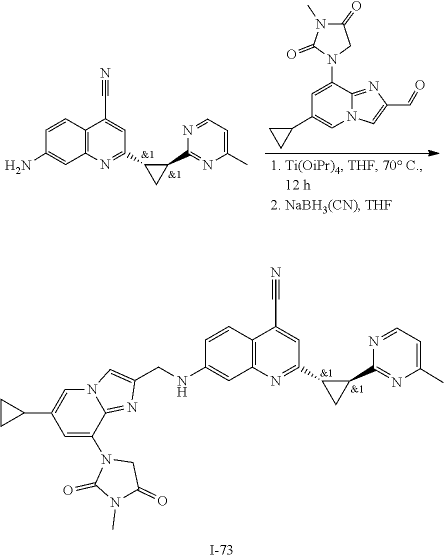

### Synthesis of 7-bromo-2-hydroxyquinoline-4-carboxylic acid

A mixture of 6-bromoisatin (22.6 g, 100 mmol) and malonic acid (52 g, 500 mmol) in AcOH (300 mL) was refluxed for 12 h. After cooling to room temperature, the reaction mixture was poured into water (1 L). The mixture was filtered, and the filter cake was washed with water to give a brown solid. The solid was stirred and heated in NaHCO₃ (sat. aq., 100 mL) to give a near-clear solution, the insoluble material was filtered off. The filtrate was collected and acidified to pH=1 with concentrated HCl, solid formed and filtered, the filter cake was washed with water (100 mL×3) and dried in vacuo to give 7-bromo-2-hydroxyquinoline-4-carboxylic acid (20 g, 75%) as a brown solid.

ESI-MS [M+H]⁺: 267.9.

### Synthesis of 7-bromo-2-chloroquinoline-4-carboxamide

A solution of 7-bromo-2-hydroxy-4-quinolinecarboxylic acid (20 g, 75 mmol) in POCl₃ (50 mL) was refluxed for 5 h. After cooling to room temperature, the reaction mixture was concentrated in vacuo to give a black solid, which was added to a solution of NH₃ in i-PrOH (300 mL, 2 M) at 0° C. in portions. The resulting mixture was concentrated in vacuo to give the crude, which was purified by column chromatography (eluent: MeOH/DCM=0˜5%) to give the 7-bromo-2-chloroquinoline-4-carboxamide (10 g, 47%) as a white solid. ESI-MS [M+H]⁺: 286.9.

### Synthesis of 7-bromo-2-chloroquinoline-4-carbonitrile

To a mixture of 7-bromo-2-chloroquinoline-4-carboxamide (10 g, 35 mmol) and TEA (20 ml) in THF (100 mL) was added TFAA (10 mL) at 0° C., the resulting mixture was stirred at 0° C. for 2 h. The reaction mixture was quenched with NaHCO₃ (sat. aq., 200 mL) and extracted with EtOAc (3×100 mL). The combined organic layers were washed with brine (100 mL), dried over Na₂SO₄ and concentrated in vacuo to give the crude, which was purified by column chromatography (eluent: MeOH/DCM=0˜2%) to give the 7-bromo-2-chloroquinoline-4-carbonitrile (6 g, 64%) as yellow solid. ESI-MS [M+H]⁺: 268.9.

### Synthesis of 3-(2-(4-methylpyrimidin-2-yl)cyclopropyl)quinoxalin-6-amine

To a mixture of 7-bromo-2-chloroquinoline-4-carbonitrile (4.0 g, 15 mmol), (E)-4-methyl-2-(2-(4,4,5,5-tetramethyl-1,3,2-dioxaborolan-2-yl)vinyl)pyrimidine (3.3 g, 13.4 mmol) and K₃PO₄ (8.5 g, 40.2 mmol) in THF/H₂O (100/10 mL) was added Pd(PPh₃)₂Cl₂ (470 mg, 0.67 mol). The mixture was stirred at room temperature for 5 h under N₂. The mixture was poured into water (300 mL) and extracted with EtOAc (200 mL×3). The combined organic layers were washed with brine (300 mL), dried over Na₂SO₄, and concentrated in vacuo to give the crude, which was purified by column chromatography (eluent: MeOH/DCM=0˜2%) to give (E)-7-bromo-2-(2-(4-methylpyrimidin-2-yl)vinyl)quinoline-4-carbonitrile (2.5 g, 53%) as a yellow solid. ESI-MS [M+H]+: 351.0.

### Synthesis of rac-7-bromo-2-((1S*2S*)-2-(4-methylpyrimidin-2-yl)cyclopropyl)quinoline-4-carbonitrile

To a stirred solution of NaH (860 mg, 60% in mineral oil, 21.5 mmol) in DMSO (12 mL) was added trimethylsulfoxonium iodide (4.7 g, 21.5 mmol) portionwise at 0° C., the resulting mixture was stirred at room temperature for 1 h under N₂. A solution of (E)-7-bromo-2-(2-(4-methylpyrimidin-2-yl)vinyl)quinoline-4-carbonitrile (1.5 g, 4.3 mmol) in DMSO (8 mL) was added in and the reaction mixture was stirred at room temperature for 2 h. The mixture was poured into water (100 mL) and extracted with EtOAc (3×100 mL). The combined organic layers were washed with brine (50 mL), dried over Na₂SO₄, and concentrated in vacuo to give the crude, which was purified by column chromatography (eluent: MeOH/DCM=0˜2%) to give the rac-7-bromo-2-((1S*,2S*)-2-(4-methylpyrimidin-2-yl)cyclopropyl)quinoline-4-carbonitrile (200 mg, 13%) as a yellow solid. ESI-MS [M+H]+: 365.0

### Synthesis of rac-tert-butyl (4-cyano-2-((7S*,2S*)-2-(4-methylpyrimidin-2-yl)cyclopropyl)quinolin-7-yl)carbamate

To a stirred solution of rac-7-bromo-2-((1S*,2S*)-2-(4-methylpyrimidin-2-yl)cyclopropyl)quinoline-4-carbonitrile (120 mg, 0.33 mmol), BocNH₂ (77 mg, 0.66 mmol), Xphos (33 mg, 0.07 mmol), Cs₂CO₃ (323 mg, 0.99 mmol) in 1,4-dioxane (15 mL) was added Pd(OAc)₂ (11 mg, 0.05 mmol). Then the reaction was stirred at 90° C. for 5 h under N₂. The reaction mixture was filtered through Celite® and the filter cake was washed with DCM/MeOH (10/1, 30 mL). The filtrate was concentrated in vacuo to give the crude, which was purified by column chromatography (eluent: MeOH/DCM=0˜2%) to give rac-tert-butyl (4-cyano-2-((1S*,2S*)-2-(4-methylpyrimidin-2-yl)cyclopropyl)quinolin-7-yl)carbamate (80 mg, 61%) as yellow solid. ESI-MS [M+H]+: 402.2.

### Synthesis of rac-7-amino-2-((1S*,2S*)-2-(4-methylpyrimidin-2-yl)cyclopropyl)quinoline-4-carbonitrile

To a reaction mixture of rac-tert-butyl (4-cyano-2-((1S*,2S*)-2-(4-methylpyrimidin-2-yl)cyclopropyl)quinolin-7-yl)carbamate (80 mg, 0.2 mol) in DCM (10 mL) was added TFA (1 mL). The reaction was stirred at room temperature for 16 h. The reaction was quenched with NaHCO₃ (sat. aq., 30 mL) and extracted with DCM (20 mL×3). The combined organic layers were washed with brine (30 mL), dried over Na₂SO₄ then concentrated in vacuo to give the crude, which was purified by preparative TLC (eluent: MeOH/DCM=1/20) to afford rac-7-amino-2-((1S*,2S*)-2-(4-methylpyrimidin-2-yl)cyclopropyl)quinoline-4-carbonitrile (40 mg, 67%) as a yellow solid. ESI-MS [M+H]⁺: 302.2.

### Synthesis of rac-7-(((6-cyclopropyl-8-(3-methyl-2,4-dioxoimidazolidin-1-yl)imidazo[1,2-a]pyridin-2-yl)methyl)amino)-2-((1S*,2S*)-2-(4-methylpyrimidin-2-yl)cyclopropyl)quinoline-4-carbonitrile

To a reaction mixture of rac-7-amino-2-((1S*,2S*)-2-(4-methylpyrimidin-2-yl)cyclopropyl)quinoline-4-carbonitrile (40 mg, 0.13 mmol) and 6-cyclopropyl-8-(3-methyl-2,4-dioxoimidazolidin-1-yl)imidazo[1,2-a]pyridine-2-carbaldehyde (43 mg, 0.14 mol) in THF (10 mL) was added Ti(Oi-Pr)₄ (185 mg, 0.65 mmol). The resulting solution was stirred 12 h at 70° C. After the reaction was cooled to room temperature, MeOH (1 mL) and NaBH₃CN (25 mg, 0.39 mmol) were added and the mixture was stirred at room temperature for an additional 1 hour. The reaction was quenched with water (30 mL) and extracted with EtOAc (30 mL×3). The combined organic layers were washed with brine (30 mL), dried over Na₂SO₄, concentrated in vacuo to give the crude, which was purified by preparative TLC (eluent: DCM/MeOH=40/1) to give rac-7-(((6-cyclopropyl-8-(3-methyl-2,4-dioxoimidazolidin-1-yl)imidazo[1,2-a]pyridin-2-yl)methyl)amino)-2-((1S*,2S*)-2-(4-methylpyrimidin-2-yl)cyclopropyl)quinoline-4-carbonitrile (15 mg, 20%) as yellow solid.

ESI-MS [M+H]⁺: 584.2, ¹H NMR (400 MHz, DMSO) δ 8.52 (d, J=5.1 Hz, 1H), 8.25 (s, 1H), 7.78-7.69 (m, 3H), 7.35 (d, J=1.4 Hz, 1H), 7.28-7.24 (m, 2H), 7.19 (d, J=5.1 Hz, 1H), 6.90 (d, J=2.1 Hz, 1H), 4.93 (s, 2H), 4.52 (d, J=5.6 Hz, 2H), 2.97 (s, 3H), 2.80-2.73 (m, 2H), 2.41 (s, 3H), 1.99-1.89 (m, 1H), 1.82-1.74 (m, 2H), 0.95-0.90 (m, 2H), 0.66-0.62 (m, 2H).

### Example 74

### Synthesis of rac-N-((6-cyclopropyl-8-(4-ethylpiperazin-1-yl)imidazo[1,2-a]pyridin-2-yl) methyl)-2-((1S*,2S*)-2-(4-methylpyrimidin-2-yl)cyclopropyl)quinolin-7-amine (I-74)

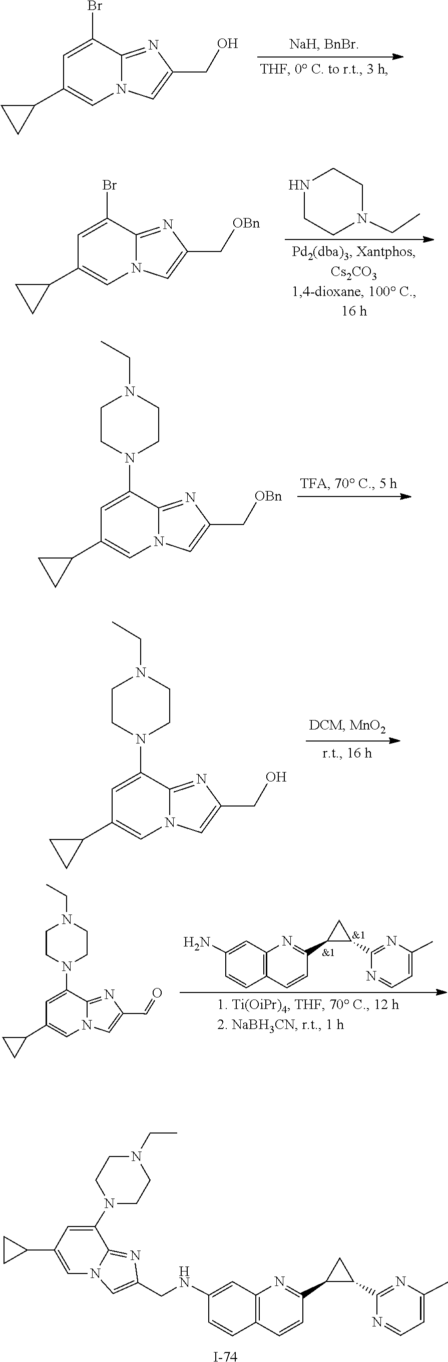

### Synthesis of 2-((benzyloxy)methyl)-8-bromo-6-cyclopropylimidazo[1,2-a]pyridine

To a solution of (8-bromo-6-cyclopropylimidazo[1,2-a]pyridin-2-yl)methanol (500 mg, 1.88 mmol) in THF (10 mL) was added NaH (60% in mineral oil, 150 mg, 3.76 mmol) at 0° C. The reaction mixture was stirred at 0° C. for 1 h under N₂, then a solution of (bromomethyl)benzene (320 mg, 1.88 mmol) in THF (5 mL) was added. The mixture was stirred at room temperature for 2 h. The reaction was quenched with water (50 mL) and extracted with EtOAc (30 mL×3). The combined organic layers were washed with brine (50 mL), dried over anhydrous Na₂SO₄, and concentrated to give the crude product, which was purified by column chromatography (eluent: PE/EtOAc=5/1) to give 2-((benzyloxy)methyl)-8-bromo-6-cyclopropylimidazo[1,2-a]pyridine (500 mg, 75%) as a yellow oil.

ESI-MS [M+H]+: 357.2.

### Synthesis of 2-((benzyloxy)methyl)-6-cyclopropyl-8-(4-ethylpiperazin-1-yl)imidazo[1,2-a]pyridine

To a mixture of 2-((benzyloxy)methyl)-8-bromo-6-cyclopropylimidazo[1,2-a]pyridine (200 mg, 0.56 mmol), 1-ethylpiperazine (205 mg, 1.8 mmol) and Cs₂CO₃ (456 mg, 1.4 mmol) in 1,4-dioxane (10 mL) was added Pd₂(dba)₃ (42 mg, 0.046 mmol) and Xantphos (53 mg, 0.092 mmol). The resulting mixture was stirred at 100° C. for 16 h under N₂. The reaction mixture was filtered through Celite® and the filter cake was washed with DCM/MeOH (10/1, 30 mL). The filtrate was concentrated in vacuo to give the crude, which was purified by preparative TLC, eluting with 10% MeOH/DCM to give 2-((benzyloxy)methyl)-6-cyclopropyl-8-(4-ethylpiperazin-1-yl)imidazo [1,2-a]pyridine (130 mg, 59%) as a yellow solid. ESI-MS [M+H]⁺: 391.2.

### Synthesis of (6-cyclopropyl-8-(4-ethylpiperazin-1-yl)imidazo[1,2-a]pyridin-2-yl)methanol

A solution of 2-((benzyloxy)methyl)-6-cyclopropyl-8-(4-ethylpiperazin-1-yl)imidazo[1,2-a]pyridine (120 mg, 0.31 mmol) in TFA (5.0 mL) was stirred at 70° C. under N₂ for 5 h. The reaction mixture was concentrated in vacuo to give a crude, which was diluted with NaHCO₃ (sat. aq., 30 L), extracted with EtOAc (30 mL×3). The combined organic layers were washed with brine (30 mL), dried over Na₂SO₄ then concentrated in vacuo to give the crude, which was purified with column chromatography on silica gel, eluting with 0˜10% MeOH/DCM to give (6-cyclopropyl-8-(4-ethylpiperazin-1-yl)imidazo[1,2-a] pyridin-2-yl)methanol (70 mg, 75%) as a yellow solid. ESI-MS [M+H]⁺: 301.2.

### Synthesis of 6-cyclopropyl-8-(4-ethylpiperazin-1-yl)imidazo[1,2-a]pyridine-2-carbaldehyde

A mixture of (6-cyclopropyl-8-(4-ethylpiperazin-1-yl)imidazo[1,2-a]pyridin-2-yl)methanol (70 mg, 0.23 mmol) and MnO₂ (200 mg, 2.3 mmol) in DCM (10 mL) was stirred at room temperature for 16 h. The reaction mixture was filtered through Celite® and the filter cake was washed with DCM/MeOH (10/1, 30 mL). The filtrate was concentrated in vacuo to give the crude, which was purified by preparative TLC, eluting with 10% MeOH/DCM to give 6-cyclopropyl-8-(4-ethylpiperazin-1-yl)imidazo[1,2-a]pyridine-2-carbaldehyde (55 mg, 80%) as a yellow solid. ESI-MS [M+H]⁺: 299.2.

### Synthesis of rac-N-((6-cyclopropyl-8-(4-ethylpiperazin-1-yl)imidazo[1,2-a]pyridin-2-yl)methyl)-2-((1S*,2S*)-2-(4-methylpyrimidin-2-yl)cyclopropyl)quinolin-7-amine

A mixture of 6-cyclopropyl-8-(4-ethylpiperazin-1-yl)imidazo[1,2-a]pyridine-2-carbaldehyde (60 mg, 0.20 mmol), rac-2-((1S*,2S*)-2-(4-methylpyrimidin-2-yl)cyclopropyl)quinolin-7-amine (55 mg, 0.20 mmol) and Ti(O-iPr)₄ (280 mg, 0.99 mmol) in THF (10 mL) was stirred at 70° C. for 12 h under N₂. After cooling to 0° C., the mixture was added MeOH (2.0 mL) and NaBH₃CN (38 mg, 0.60 mmol) subsequently. The resulting mixture was stirred at room temperature for another 1 h. The reaction was quenched with water (20 mL) and then extracted with EtOAc (20 mL×3). The combined organic layers were washed with brine (20 mL), dried over anhydrous Na₂SO₄, and concentrated in vacuo to give the crude, which was purified by preparative TLC, eluting with 10% MeOH/DCM to give rac-N-((6-cyclopropyl-8-(4-ethylpiperazin-1-yl)imidazo[1,2-a]pyridin-2-yl) methyl)-2-((1S*,2S*)-2-(4-methylpyrimidin-2-yl)cyclopropyl)quinolin-7-amine (18 mg, 16%) as a yellow solid.

ESI-MS [M+H]⁺: 559.2, ¹H NMR (400 MHz, DMSO) δ 8.51 (d, J=5.1 Hz, 1H), 7.90 (d, J=8.2 Hz, 1H), 7.87 (s, 1H), 7.59 (s, 1H), 7.56 (d, J=8.9 Hz, 1H), 7.17 (d, J=5.2 Hz, 1H), 7.11 (d, J=8.2 Hz, 1H), 7.03 (dd, J=8.8, 2.1 Hz, 1H), 6.80 (d, J=1.6 Hz, 1H), 6.76 (t, J=5.6 Hz, 1H), 6.16 (s, 1H), 4.44 (d, J=5.4 Hz, 2H), 3.50 (s, 4H), 2.76-2.64 (m, 2H), 2.57 (s, 4H), 2.41 (s, 5H), 1.86-1.81 (m, 1H), 1.79-1.67 (m, 2H), 1.03 (t, J=7.1 Hz, 3H), 0.89-0.80 (m, 2H), 0.69-0.61 (m, 2H).

### Example 75

### Synthesis of rac-1-(6-cyclopropyl-2-(((5-(methoxymethyl)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (I-75)

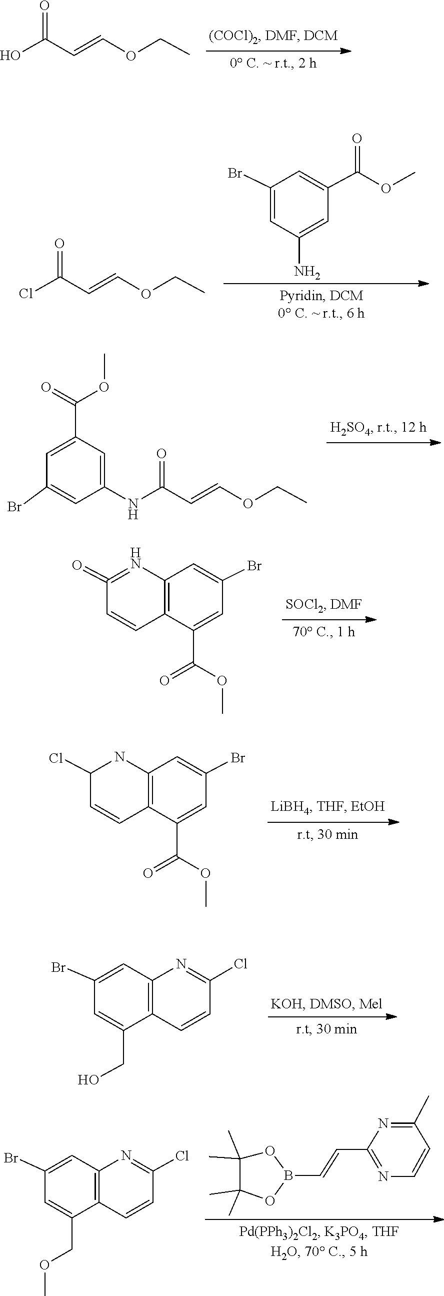

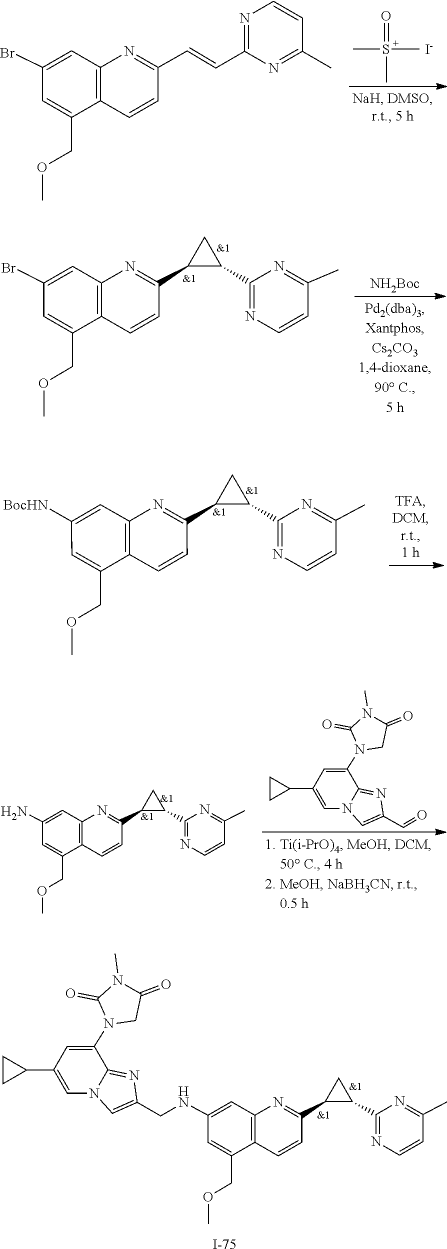

### Synthesis of (E)-3-ethoxyacryloyl chloride

To a stirred solution of (E)-3-ethoxyacrylic acid (6.1 g, 52 mmol) in DCM (60 mL) was added (COCl)₂ (8.6 g, 68 mmol) and DMF (0.10 mL) slowly at 0° C. The resulting mixture was stirred at room temperature for 1 h. The reaction mixture was concentrated and dried in vacuo to give (E)-3-ethoxyacryloyl chloride (7.0 g, quant.) as a light brown solid. ESI-MS [M+H]⁺: 131.1 (in MeOH).

### Synthesis of methyl (E)-3-bromo-5-(3-ethoxyacrylamido)benzoate

To a stirred solution of methyl 3-amino-5-bromobenzoate (6.0 g, 26 mmol) in DCM/Pyridine (10/1, 60 mL) was added a solution of (E)-3-ethoxyacryloyl chloride (7.0 g, 52 mmol) in DCM (30 mL) slowly at 0° C. The resulting mixture was stirred at room temperature for 10 h. The reaction was quenched with water (80 mL) and then concentrated in vacuo to remove volatiles. To the remaining residue was added HCl (3 N in H₂O, 10 mL,), stirred for another 10 min, and then extracted with EtOAc (100 mL×2). The combined organic layers were washed with NaHCO₃ (sat. aq., 100 mL×2) and brine (100 mL), dried over Na₂SO₄ then concentrated in vacuo to give the crude, which was purified by column chromatography on silica gel, eluting with 5˜20% EtOAc/PE to give methyl (E)-3-bromo-5-(3-ethoxyacrylamido)benzoate (6.2 g, 73%) as a yellow solid. ESI-MS [M+H]⁺: 328.0.

### Synthesis of methyl 7-bromo-2-oxo-1,2-dihydroquinoline-5-carboxylate

A solution of methyl (E)-3-bromo-5-(3-ethoxyacrylamido)benzoate (6.2 g, 19 mmol) in conc. H₂SO₄ (30 mL) was stirred at room temperature for 12 h. The reaction mixture was poured into ice-water (300 mL) to afford a precipitate, which was isolated by filtration and triturated with EtOAc (100 mL). The filter cake was dried in vacuo to give methyl 7-bromo-2-oxo-1,2-dihydroquinoline-5-carboxylate (5.3 g, quant) as a yellow solid. ESI-MS [M+H]⁺: 282.0.

### Synthesis of methyl 7-bromo-2-chloroquinoline-5-carboxylate

A mixture of methyl 7-bromo-2-oxo-1,2-dihydroquinoline-5-carboxylate (2.5 g, 8.9 mmol) and DMF (0.78 g, 11 mmol) in SOCl₂ (20 mL) was stirred at 70° C. for 1 h. The reaction mixture was concentrated in vacuo. The residue was diluted with water (100 mL), extracted with EtOAc (100 mL×2). washed with brine (100 mL×1) then dried over Na₂SO₄. The combined organic layers were concentrated in vacuo to give the crude, which was purified by column chromatography on silica gel, eluting with 0˜10% EtOAc/PE to give methyl 7-bromo-2-chloroquinoline-5-carboxylate (1.9 g, 71%) as a white solid. ESI-MS [M+H]⁺: 300.0.

### Synthesis of (7-bromo-2-chloroquinolin-5-yl)methanol

To a stirred solution of 7-bromo-2-chloroquinoline-5-carboxylate (1.9 g, 6.3 mmol) in THF (60 mL) and EtOH (5.0 mL) was added LiBH₄ (0.27 g, 13 mmol) in portions at 0° C. The resulting mixture was stirred at room temperature for 0.5 h. The reaction was quenched with NH₄Cl (sat. aq., 20 mL) and then extracted with EtOAc (50 mL×2). The combined organic layers were washed with brine (80 mL×1), dried over Na₂SO₄, and then concentrated in vacuo to give (7-bromo-2-chloroquinolin-5-yl)methanol (1.7 g, quant) as a white solid. ESI-MS [M+H]⁺: 271.9.

### Synthesis of 7-bromo-2-chloro-5-(methoxymethyl)quinoline

To a stirred solution of (7-bromo-2-chloroquinolin-5-yl)methanol (1.7 g, 6.3 mmol) in DMSO (30 mL) was added KOH (1.1 g, 19 mmol). After stirred at room temperature for 10 min, MeI (4.4 g, 31 mmol) was added dropwise and the resulting mixture was stirred at room temperature for 5 h. Water (150 mL) was added and extracted with EtOAc (100 mL×3). The combined organic layers were washed with brine (200 mL), dried over Na₂SO₄, and then concentrated in vacuo to give the crude, which was purified by column chromatography on silica gel, eluting with 0˜10% EtOAc/PE to give 7-bromo-2-chloro-5-(methoxymethyl)quinoline (1.5 g, 83%) as a white solid. ESI-MS [M+H]⁺: 286.0.

### Synthesis of (E)-7-bromo-5-(methoxymethyl)-2-(2-(4-methylpyrimidin-2-yl)vinyl)quinoline

A mixture of 7-bromo-2-chloro-5-(methoxymethyl)quinoline (1.5 g, 5.2 mmol), (E)-4-methyl-2-(2-(4,4,5,5-tetramethyl-1,3,2-dioxaborolan-2-yl)vinyl)pyrimidine (1.8 g, 7.2 mmol), Pd(PPh₃)₂Cl₂ (0.38 g, 0.52 mmol) and K₃PO₄ (3.2 g, 15 mmol) in THF (30 mL) and H₂O (3.0 mL) was stirred at 70° C. for 10 h under N₂. After the reaction was cooled to room temperature, water (100 mL) was added and extracted with EtOAc (100 mL×3). The combined organic layers were washed with brine (100 mL), dried over Na₂SO₄, and then concentrated in vacuo to give the crude, which was purified by column chromatography on silica gel, eluting with 10˜30% EtOAc/PE to give (E)-7-bromo-5-(methoxymethyl)-2-(2-(4-methylpyrimidin-2-yl)vinyl)quinoline (1.3 g, 68%) as a yellow solid. ESI-MS [M+H]⁺: 370.1.

### Synthesis of rac-7-bromo-5-(methoxymethyl)-2-((1S*,2S*)-2-(4-methylpyrimidin-2-yl)cyclopropyl)quinoline

To a stirred suspension of NaH (0.40 g, 60% in mineral oil, 10 mmol) in DMSO (15 mL) was added trimethylsulfoxonium iodide (2.2 g, 10 mmol) in portions. The mixture was stirred at room temperature for 1 h until a clear solution was obtained. Then a solution of (E)-7-bromo-5-(methoxymethyl)-2-(2-(4-methylpyrimidin-2-yl)vinyl)quinoline (1.2 g, 3.3 mmol) in DMSO (5 mL) was added. The resulting mixture was stirred at room temperature for 5 h. The reaction mixture was poured into water (60 mL) and then extracted with EtOAc (50 mL×3). The combined organic layers were washed with brine (80 mL×1), dried over Na₂SO₄, and then concentrated in vacuo to give the crude, which was purified by column chromatography on silica gel, eluting with 0˜20% EtOAc/PE to give rac-7-bromo-5-(methoxymethyl)-2-((1S*,2S*)-2-(4-methylpyrimidin-2-yl)cyclopropyl)quinoline (0.45 g, 35%) as a yellow solid. ESI-MS [M+H]⁺: 384.1.

### Synthesis of rac-tert-butyl (5-(methoxymethyl)-2-((1S*,2S*)-2-(4-methylpyrimidin-2-yl)cyclopropyl)quinolin-7-yl)carbamate

A mixture of rac-7-bromo-5-(methoxymethyl)-2-((1S*,2S*)-2-(4-methylpyrimidin-2-yl)cyclopropyl)quinoline (0.46 g, 1.2 mmol), tert-butyl carbamate (0.27 g, 2.3 mmol), Pd₂(dba)₃ (0.11 g, 0.12 mmol), Xantphos (0.13 g, 0.23 mmol) and Cs₂CO₃ (1.1 g, 3.5 mmol) in 1,4-dioxane (20 mL) was stirred at 90° C. for 4 h under N₂. The reaction mixture was diluted in DCM/MeOH (10/1, 50 mL) then filtered. The filtrate was concentrated in vacuo to give the crude, which was purified by column chromatography on silica gel, eluting with 0˜20% EtOAc/PE to give rac-tert-butyl (5-(methoxymethyl)-2-((1S*,2S*)-2-(4-methylpyrimidin-2-yl)cyclopropyl)quinolin-7-yl)carbamate (0.35 g, 70%) as a yellow syrup. ESI-MS [M+H]⁺: 421.2.

### Synthesis of rac-5-(methoxymethyl)-2-((1S*,2S*)-2-(4-methylpyrimidin-2-yl)cyclopropyl)quinolin-7-amine

To a stirred solution of rac-tert-butyl (5-(methoxymethyl)-2-((1S*,2S*)-2-(4-methylpyrimidin-2-yl)cyclopropyl)quinolin-7-yl)carbamate (0.31 g, 0.74 mmol) in DCM (10 mL) was added TFA (2 mL). The resulting mixture was stirred at room temperature for 1 h. The reaction mixture was concentrated in vacuo to remove volatile. The residue was diluted with water (30 mL) and pH of the resulting mixture was adjusted to 9˜10 by NaHCO₃ (sat. aq., 10 mL), then extracted with EtOAc (50 mL×3). The combined organic layers were washed with brine (60 mL), dried over Na₂SO₄, and then concentrated in vacuo to give the crude, which was purified by column chromatography on silica gel, eluting with 0˜50% EtOAc/PE to give rac-5-(methoxymethyl)-2-((1S*,2S*)-2-(4-methylpyrimidin-2-yl)cyclopropyl) quinolin-7-amine (0.20 g, 84%) as a yellow solid. ESI-MS [M+H]⁺: 321.2.

### Synthesis of rac-1-(6-cyclopropyl-2-(((5-(methoxymethyl)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione

To a stirred solution of rac-5-(methoxymethyl)-2-((1S*,2S*)-2-(4-methylpyrimidin-2-yl)cyclopropyl) quinolin-7-amine (81 mg, 0.25 mmol) and 6-cyclopropyl-8-(3-methyl-2,4-dioxoimidazolidin-1-yl)imidazo[1,2-a]pyridine-2-carbaldehyde (77 mg, 0.26 mmol) in MeOH (5.0 mL) and DCM (5.0 mL) was added Ti(i-PrO)₄ (0.36 g, 1.3 mmol). The resulting mixture was stirred at 50° C. for 4 h then cooled to room temperature. NaBH₃CN (80 mg, 1.27 mmol) was added in portions and stirred at room temperature for another 0.5 h. The reaction mixture was diluted in water (50 mL) and extracted with EtOAc (50 mL×2). The combined organic layers were washed with brine (80 mL), dried over Na₂SO₄, and then concentrated in vacuo to give the crude, which was purified by column chromatography on silica gel, eluting with 0˜50% EtOAc/PE to give rac-1-(6-cyclopropyl-2-(((5-(methoxymethyl)-2-((1S*)-2-(4-methylpyrimidin-2-yl)cyclopropyl)quinolin-7-yl)amino)methyl)imidazo[1,2-a] pyridin-8-yl)-3-methylimidazolidine-2,4-dione (0.11 g, 73%) as a yellow solid.

ESI-MS [M+H]⁺: 603.3. ¹H NMR (400 MHz, DMSO) δ 8.50 (d, J=5.1 Hz, 1H), 8.24 (d, J=1.0 Hz, 1H), 8.03 (d, J=8.5 Hz, 1H), 7.74 (s, 1H), 7.34 (d, J=1.5 Hz, 1H), 7.14 (dd, J=13.1, 6.8 Hz, 2H), 7.08 (d, J=2.2 Hz, 1H), 6.82 (t, J=5.8 Hz, 1H), 6.70 (d, J=2.0 Hz, 1H), 4.94 (s, 2H), 4.70 (s, 2H), 4.48 (d, J=5.7 Hz, 2H), 3.32 (s, 3H), 2.96 (s, 3H), 2.71-2.64 (m, 2H), 2.40 (s, 3H), 1.96-1.87 (m, 1H), 1.77-1.68 (m, 2H), 0.94-0.88 (m, 2H), 0.65-0.60 (m, 2H).

### Example 76

### Synthesis of rac-1-(6-cyclopropyl-2-(((4-(methoxymethyl)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (I-76)

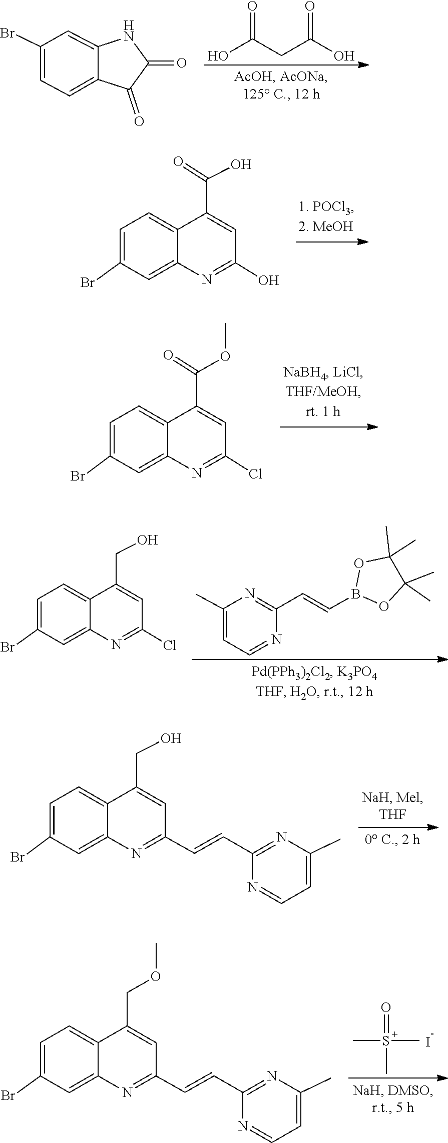

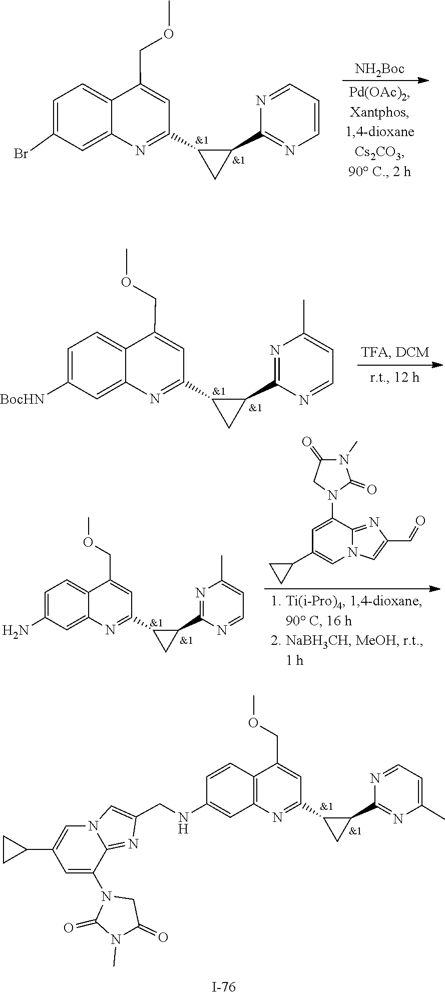

### Synthesis of 7-bromo-2-hydroxyquinoline-4-carboxylic acid

A mixture of 6-bromoindoline-2,3-dione (5.65 g, 25 mmol), malonic acid (2.86 g, 27.5 mmol) and AcONa (4.1 g, 50 mmol) in AcOH (60 mL) was stirred at 125° C. for 12 h. The reaction was concentrated in vacuo. Water (100 mL) was added to the residue and the mixture was stirred for 10 min then filtered and washed with water (150 mL). The filter cake was dried to give the crude product 7-bromo-2-hydroxyquinoline-4-carboxylic acid (6.0 g, crude) as a brown solid. ESI-MS [M+H]+: 269.2

### Synthesis of methyl 7-bromo-2-chloroquinoline-4-carboxylate

A solution of 7-bromo-2-hydroxyquinoline-4-carboxylic acid (6.0 g, crude) in POCl₃ (40 mL) was stirred at 115° C. for 2 h. Then the mixture was cooled to room temperature and concentrated in vacuo. The residue was dissolved in MeOH (100 mL) and stirred at room temperature for 20 min. Then the mixture was concentrated in vacuo to give the crude product, which was purified by silica gel chromatography (elute: PE/EA=4:1) to give the product methyl 7-bromo-2-chloroquinoline-4-carboxylate as a yellow solid. (4.5 g, 60% over two steps). ESI-MS [M+H]+: 301.1

### Synthesis of (7-bromo-2-chloroquinolin-4-yl)methanol

To a mixture of methyl 7-bromo-2-chloroquinoline-4-carboxylate (4.5 g, 15 mmol) and LiCl (630 mg, 15 mmol) in THF/MeOH (60 mL/20 mL) was added NaBH₄ (1.14 g, 30 mmol) at 0° C. The mixture was stirred at room temperature for 2 h. The reaction was concentrated in vacuo and the residue was adjusted the pH to 7 with HCl (1M aq.). The mixture was extracted with EtOAc (50 mL×3). The combined organic layers were washed with brine (50 mL), dried over Na₂SO₄, concentrated in vacuo to give the crude product, which was purified with flash chromatography (elute: PE/EA=3:1) to give the product (7-bromo-2-chloroquinolin-4-yl)methanol as a yellow solid. (3.06 g, 75%). ESI-MS [M+H]+: 273.2

### Synthesis of (E)-(7-bromo-2-(2-(4-methylpyrimidin-2-yl)vinyl)quinolin-4-yl)methanol

A solution of (7-bromo-2-chloroquinolin-4-yl)methanol (2.6 g, 9.7 mmol), (E)-4-methyl-2-(2-(4,4,5,5-tetramethyl-1,3,2-dioxaborolan-2-yl)vinyl)pyrimidine (2.0 g, 8.1 mmol), PdCl₂(PPh₃) (0.60 g, 0.81 mmol) and K₃PO₄ (5.1 g, 24 mmol) in THF/H₂O (3/1, 33 mL) was stirred at room temperature for 12 h. The reaction mixture was then filtered and the filtrate was concentrated in vacuo to give the crude, which was purified with column chromatography on silica gel, eluting with 50% PE/EtOAc to give (E)-(7-bromo-2-(2-(4-methylpyrimidin-2-yl)vinyl)quinolin-4-yl)methanol (0.84 g, 29%) as a yellow solid.

ESI-MS [M+H]+: 356.2

### Synthesis of (E)-7-bromo-4-(methoxymethyl)-2-(2-(4-methylpyrimidin-2-yl)vinyl)quinoline

To a solution of (E)-(7-bromo-2-(2-(4-methylpyrimidin-2-yl)vinyl)quinolin-4-yl)methanol (0.84 g, 2.4 mmol) in THF (20 mL) was added NaH (0.10 g, 60% in mineral oil, 2.5 mmol) at 0° C. After stirring at room temperature for 1 h, MeI (1.7 g, 12 mmol) was added slowly and the resulting mixture was stirred at room temperature for another 1 h. The reaction was quenched with H₂O (30 mL) and extracted with EtOAc (50 mL×3). The combined organic layers were washed with brine (50 mL), dried over Na₂SO₄, and then concentrated in vacuo to give the crude, which was purified by column chromatography on silica gel, eluting with 50% PE/EtOAc to give (E)-7-bromo-4-(methoxymethyl)-2-(2-(4-methylpyrimidin-2-yl)vinyl)quinoline (0.49 g, 55%) as a yellow solid.

ESI-MS [M+H]+: 370.1

### Synthesis of rac-7-bromo-4-(methoxymethyl)-2-((1S*,2S*)-2-(4-methylpyrimidin-2-yl)cyclopropyl) quinoline

A mixture of trimethylsulfoxonium iodide (0.86 g, 3.9 mmol) and NaH (0.16 g, 60% in mineral oil, 3.9 mmol) in DMSO (10 mL) was stirred at room temperature for 1 h. A solution of (E)-7-bromo-4-(methoxymethyl)-2-(2-(4-methylpyrimidin-2-yl)vinyl)quinoline (0.49 g, 1.3 mmol) in DMSO (3.0 mL) was added. After the resulting mixture was stirred at room temperature for 4 h, the reaction was quenched with H₂O (50 mL) and extracted with EtOAc (50 mL×3). The combined organic layers were washed with brine (50 mL), dried over Na₂SO₄, and then concentrated in vacuo to give the crude, which was purified by column chromatography on silica gel, eluting with 30% PE/EtOAc to give rac-7-bromo-4-(methoxymethyl)-2-((1S*,2S*)-2-(4-methylpyrimidin-2-yl)cyclopropyl) quinoline (0.18 g, 36%) as a yellow solid. ESI-MS [M+H]+: 384.1

### Synthesis of rac-tert-butyl (4-(methoxymethyl)-2-((1S*,2S*)-2-(4-methylpyrimidin-2-yl)cyclopropyl)quinolin-7-yl)carbamate

A mixture of rac-7-bromo-4-(methoxymethyl)-2-((1S*,2S*)-2-(4-methylpyrimidin-2-yl)cyclopropyl) quinoline (0.18 g, 0.47 mmol), NH₂Boc (82 mg, 0.71 mmol), Pd(OAc)₂ (21 mg, 0.094 mmol) and Xantphos (54 mg, 0.094 mmol) in dioxane (15 mL) was stirred at 90° C. for 2 h. The reaction mixture was then filtered and the filtrate was concentrated in vacuo to give the crude, which was purified by preparative TLC, eluting with 30% PE/EtOAc to give rac-tert-butyl (4-(methoxymethyl)-2-((1S*,2S*)-2-(4-methylpyrimidin-2-yl)cyclopropyl)quinolin-7-yl)carbamate (0.13 g, 66%) as a white solid. ESI-MS [M+H]+: 421.2

### Synthesis of rac-4-(methoxymethyl)-2-((1S*,2S*)-2-(4-methylpyrimidin-2-yl)cyclopropyl)quinolin-7-amine

To a solution of rac-tert-butyl (4-(methoxymethyl)-2-((1S*,2S*)-2-(4-methylpyrimidin-2-yl)cyclopropyl)quinolin-7-yl)carbamate (0.13 g, 0.31 mmol) in DCM (10 mL) was added TFA (1.0 mL). After stirring at room temperature for 12 h, the resulting mixture was concentrated in vacuo and the residue was dissolved in NH₃ (7 N in MeOH, 5 mL). The mixture was then concentrated in vacuo to give a crude, which was purified by preparative TLC, eluting with 0˜10% MeOH/DCM to give rac-4-(methoxymethyl)-2-((1S*,2S*)-2-(4-methylpyrimidin-2-yl)cyclopropyl)quinolin-7-amine (88 mg, 88%) as a yellow solid. ESI-MS [M+H]+: 321.2

### Synthesis of rac-1-(6-cyclopropyl-2-(((4-(methoxymethyl)-2-((1S*,2S*)-2-(4-methylpyrimidin-2-yl) cyclopropyl)quinolin-7-yl)amino)methyl)imidazo[1,2-a]pyridin-8-yl)-3-methylimidazolidine-2,4-dione

To a mixture of rac-4-(methoxymethyl)-2-((1S*,2S*)-2-(4-methylpyrimidin-2-yl)cyclopropyl) quinolin-7-amine (88 mg, 0.28 mmol) and 6-cyclopropyl-8-(3-methyl-2,4-dioxoimidazolidin-1-yl)imidazo[1,2-a]pyridine-2-carbaldehyde (83 mg, 0.28 mmol) in dioxane (10 mL) was added Ti(OiPr)₄ (0.39 g, 1.4 mmol). The resulting mixture was stirred at 90° C. for 16 h. After cooling to room temperature, the reaction mixture was added MeOH (2 mL) and NaBH₃CN (52 mg, 0.83 mmol). The resulting mixture was stirred at room temperature for another 1 h. The reaction was then quenched with H₂O (30 mL) and then extracted with EtOAc (50 mL×3). The combined organic layers were washed with brine (50 mL), dried over Na₂SO₄, and then concentrated in vacuo to give the crude, which was purified by Prep-HPLC to give rac-1-(6-cyclopropyl-2-(((4-(methoxymethyl)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (35 mg, 21%) as a yellow solid.

ESI-MS [M+H]+: 603.3, ¹H NMR (400 MHz, DMSO) δ 8.51 (d, J=5.1 Hz, 1H), 8.25 (s, 1H), 7.75 (s, 1H), 7.71 (d, J=9.1 Hz, 1H), 7.35 (d, J=1.1 Hz, 1H), 7.17 (d, J=5.1 Hz, 1H), 7.10 (s, 1H), 7.07 (dd, J=9.1, 2.2 Hz, 1H), 6.84-6.77 (m, 2H), 4.94 (s, 2H), 4.75 (s, 2H), 4.49 (d, J=5.6 Hz, 2H), 3.35 (s, 3H), 2.98 (s, 3H), 2.75-2.62 (m, 2H), 2.41 (s, 3H), 1.98-1.88 (m, 1H), 1.81-1.73 (m, 1H), 1.73-1.65 (m, 1H), 0.96-0.87 (m, 2H), 0.67-0.60 (m, 2H).

### Example 77

### Synthesis of rac-1-(6-cyclopropyl-2-(((4-(hydroxymethyl)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (I-77)

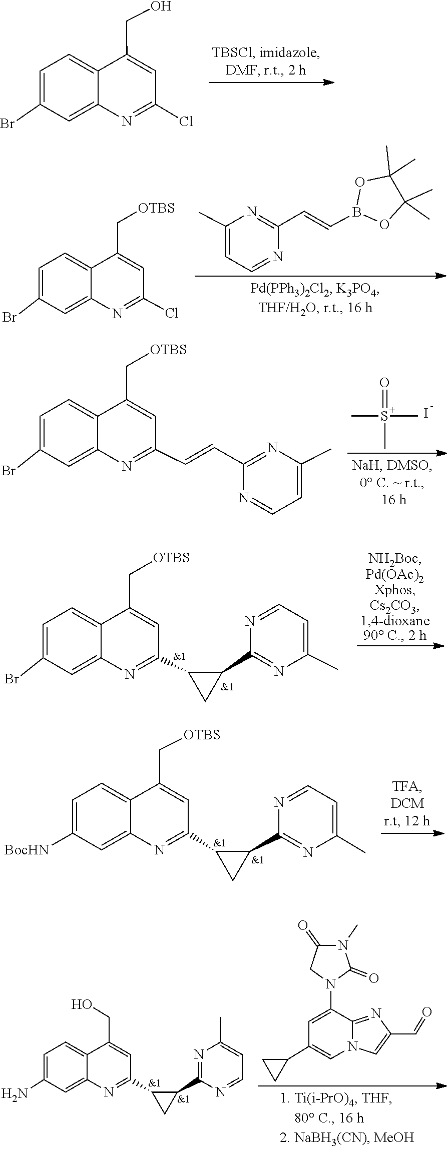

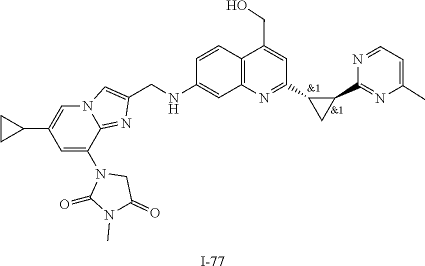

### Synthesis of 7-bromo-4-(((tert-butyldimethylsilyl)oxy)methyl)-2-chloroquinoline

To a solution of (7-bromo-2-chloroquinolin-4-yl)methanol (3.0 g, 11.0 mmol) in DMF (50 mL) was added TBSCl (4.14 g, 16.5 mmol) and imidazole (1.5 g, 11.0 mmol), the resulting mixture was stirred at room temperature for 2 h. Water (100 mL) was added and the mixture was extracted by EtOAc (100 mL×3). The combined organic layers were concentrated in vacuo to give the crude, which was purified by column chromatography (eluent: PE/EA=10/1) to give 7-bromo-4-(((tert-butyldimethylsilyl)oxy)methyl)-2-chloroquinoline (3.15 g, 74%) as a white solid. ESI-MS [M+H]+: 387.2

### Synthesis of (E)-7-bromo-4-(((tert-butyldimethylsilyl)oxy)methyl)-2-(2-(4-methylpyrimidin-2-yl)vinyl)quinoline

To a solution of 7-bromo-4-(((tert-butyldimethylsilyl)oxy)methyl)-2-chloroquinoline (2.56 g, 6.6 mmol) in THF/H₂O (50 mL/5 mL) was added (E)-4-methyl-2-(2-(4,4,5,5-tetramethyl-1,3,2-dioxaborolan-2-yl)vinyl)pyrimidine (1.49 g, 6.04 mmol), Pd(PPh₃)₂Cl₂ (423 mg, 0.6 mmol) and K₃PO₄ (3.84 g, 18.1 mmol). The reaction mixture was stirred at room temperature for 16 h under N₂. After the reaction was cooled to room temperature, water (100 mL) was added and the mixture was extracted by EtOAc (100 mL×3). The combined organic layers were concentrated in vacuo to give the crude, which was purified by column chromatography (eluent: DCM/MeOH=50/1) to give (E)-7-bromo-4-(((tert-butyldimethylsilyl)oxy)methyl)-2-(2-(4-methylpyrimidin-2-yl)vinyl)quinoline (2.5 g, 88%) as a yellow oil. ESI-MS [M+H]+: 472.1

### Synthesis of rac-7-bromo-4-(((tert-butyldimethylsilyl)oxy)methyl)-2-((1S*,2S*)-2-(4-methylpyrimidin-2-yl)cyclopropyl)quinoline

To a solution of trimethylsulfoxonium iodide (2.34 g, 10.6 mmol) in DMSO (30 mL) was added NaH (340 mg, 60% in mineral oil, 8.5 mmol) and the mixture was stirred at room temperature for 1 h. Then (E)-7-bromo-4-(((tert-butyldimethylsilyl)oxy)methyl)-2-(2-(4-methylpyrimidin-2-yl)vinyl)quinoline (1.0 g, 2.12 mmol) was added and the mixture was stirred at room temperature for 16 h. Water (100 mL) was added and the mixture was extracted by EtOAc (100 mL×3). The combined organic layers were concentrated in vacuo to give the crude, which was purified by column chromatography (eluent: DCM/MeOH=80/1) to give rac-7-bromo-4-(((tert-butyldimethylsilyl)oxy)methyl)-2-((1S*,2S*)-2-(4-methylpyrimidin-2-yl)cyclopropyl)quinoline (150 mg, 15%) as a yellow oil. ESI-MS [M+H]+: 486.2.

### Synthesis of rac-(4-(((tert-butyldimethylsilyl)oxy)methyl)-2-((1S*,2S*)-2-(4-methylpyrimidin-2-yl)cyclopropyl)quinolin-7-yl)carbamate

To a solution of rac-7-bromo-4-(((tert-butyldimethylsilyl)oxy)methyl)-2-((1S*,2S*)-2-(4-methylpyrimidin-2-yl)cyclopropyl)quinoline (130 mg, 0.27 mmol) in 1,4-dioxane (5 mL) was added NH₂Boc (47 mg, 0.40 mmol), Pd(OAc)₂ (13 mg, 0.053 mmol), Xphos (26 mg, 0.053 mmol) and Cs₂CO₃ (263 mg, 0.81 mmol). The reaction mixture was stirred at 90° C. for 2 h under N₂. The reaction mixture was filtered through Celite® and the filter cake was washed with DCM/MeOH (10/1, 20 mL). The filtrate was concentrated in vacuo to give the crude, which was purified by preparative TLC (eluent: PE/EA=2/1) to give rac-(4-(((tert-butyldimethylsilyl)oxy)methyl)-2-((1S*,2S*)-2-(4-methylpyrimidin-2-yl)cyclopropyl)quinolin-7-yl)carbamate (100 mg, 71%) as a yellow oil. ESI-MS [M+H]+: 521.2.

### Synthesis of rac-(7-amino-2-((1S*,2S*)-2-(4-methylpyrimidin-2-yl)cyclopropyl)quinolin-4-yl)methanol

To a solution of rac-(4-(((tert-butyldimethylsilyl)oxy)methyl)-2-((1S*,2S*)-2-(4-methylpyrimidin-2-yl)cyclopropyl)quinolin-7-yl)carbamate (100 mg, 0.19 mmol) in DCM (5 mL) was added TFA (0.5 mL). The reaction mixture was stirred at room temperature for 16 h then concentrated in vacuo. The pH of the mixture was adjusted to 8 by adding NH₃ (7 M in MeOH═, 5 mL). The mixture was concentrated in vacuo to give the crude, which was purified by preparative TLC (eluent: DCM/MeOH=10/1) to give rac-(7-amino-2-((1S*,2S*)-2-(4-methylpyrimidin-2-yl)cyclopropyl)quinolin-4-yl)methanol (52 mg, 90%) as a yellow oil. ESI-MS [M+H]+: 307.1

### Synthesis of rac-1-(6-cyclopropyl-2-(((4-(hydroxymethyl)-2-((1S*,2S*)-2-(4-methylpyrimidin-2-yl) cyclopropyl)quinolin-7-yl)amino)methyl)imidazo[1,2-a]pyridin-8-yl)-3-methylimidazolidine-2,4-dione

To a solution of rac-(7-amino-2-((1S*,2S*)-2-(4-methylpyrimidin-2-yl)cyclopropyl)quinolin-4-yl)methanol (52 mg, 0.17 mmol) in THF (6 mL) was added 6-cyclopropyl-8-(3-methyl-2,4-dioxoimidazolidin-1-yl)imidazo[1,2-a]pyridine-2-carbaldehyde (51 mg, 0.17 mmol) and Ti(i-PrO)₄ (241 mg, 0.85 mmol). The reaction mixture was stirred at 80° C. for 16 h. After cooled to room temperature, a mixture of MeOH (2 mL) and NaBH₃CN (32 mg, 0.509 mmol) was added. Then the mixture was stirred at room temperature for 1 h. The reaction was quenched with water (20 mL) and extracted with EtOAc (30 mL×3). The combined organic layers were washed with brine (20 mL), dried over anhydrous Na₂SO₄ and concentrated in vacuo to give the crude product, which was purified by preparative TLC (eluent: DCM/MeOH=10/1) to give rac-1-(6-cyclopropyl-2-(((4-(hydroxymethyl)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (15 mg, 15%) as a yellow solid.

ESI-MS [M+H]+: 589.3. ¹H NMR (400 MHz, DMSO) δ 8.51 (d, J=5.1 Hz, 1H), 8.24 (d, J=4.4 Hz, 2H), 7.74 (s, 1H), 7.69 (d, J=9.0 Hz, 1H), 7.34 (d, J=1.1 Hz, 1H), 7.22-7.10 (m, 2H), 7.04 (dd, J=9.1, 2.2 Hz, 1H), 6.80 (d, J=2.1 Hz, 1H), 6.75 (t, J=5.8 Hz, 1H), 5.38 (s, 1H), 4.95 (s, 2H), 4.85 (s, 2H), 4.48 (d, J=5.5 Hz, 2H), 2.98 (s, 3H), 2.72-2.60 (m, 2H), 2.41 (s, 3H), 1.96-1.89 (m, 1H), 1.82-1.74 (m, 1H), 1.73-1.65 (m, 1H), 0.95-0.90 (m, 2H), 0.69-0.57 (m, 2H).

### Example 78

### Synthesis of rac-1-(6-cyclopropyl-2-(((2-((1S*,2S*)-2-(4-methylpyrimidin-2-yl)cyclopropyl)quinolin-7-yl)amino)methyl)imidazo[1,2-a]pyridin-8-yl)-3-(oxetan-3-yl)imidazolidine-2,4-dione (I-78)

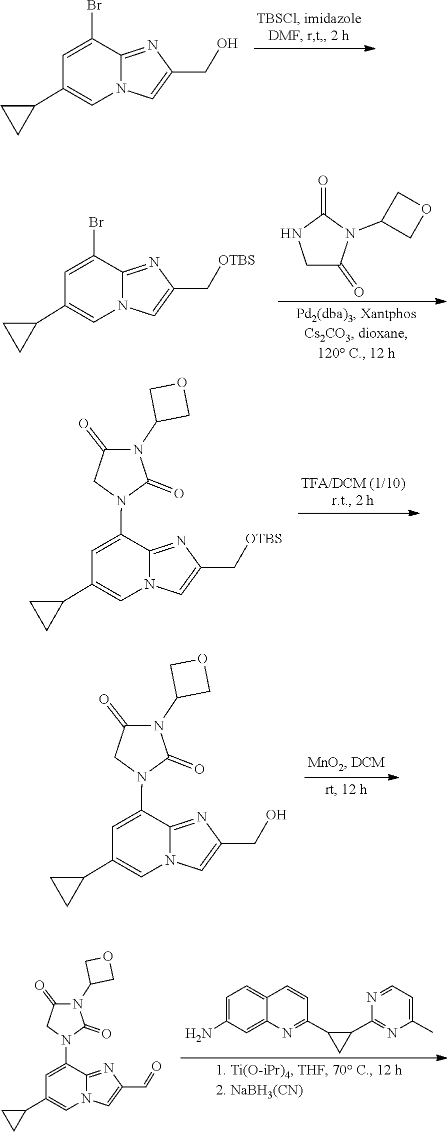

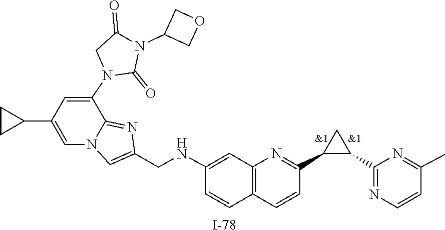

### Synthesis of 8-bromo-2-(((tert-butyldimethylsilyl)oxy)methyl)-6-cyclopropylimidazo[1,2-a]pyridine

To a mixture of (8-bromo-6-cyclopropylimidazo[1,2-a]pyridin-2-yl)methanol (2 g, 7.5 mmol) and imidazole (1.53 g, 22.5 mmol) in DMF (20 mL) was added TBSCl (1.7 g, 22.5 mmol), the mixture was stirred at room temperature for 2 h. Water (100 mL) was added and the reaction was extracted with EtOAc (30 mL×3). The combined organic layers were washed with brine (30 mL), dried over Na₂SO₄ and concentrated in vacuo. The residue was purified by column chromatography (eluent: DCM/MeOH=0˜5%) to give 8-bromo-2-(((tert-butyldimethylsilyl)oxy)methyl)-6-cyclopropylimidazo[1,2-a]pyridine (1.8 g, yield 63%) as a yellow solid. ESI-MS [M+H]⁺: 381.2.

### Synthesis of 1-(2-(((tert-butyldimethylsilyl)oxy)methyl)-6-cyclopropylimidazo[1,2-a]pyridin-8-yl)-3-(oxetan-3-yl)imidazolidine-2,4-dione

To a mixture of 8-bromo-2-(((tert-butyldimethylsilyl)oxy)methyl)-6-cyclopropylimidazo[1,2-a]pyridine (600 mg, 1.57 mmol), 3-(oxetan-3-yl)imidazolidine-2,4-dione (734 mg, 4.71 mmol) in 1,4-dioxane (10 mL) was added Pd₂(dba)₃ (137 mg, 0.15 mmol), xantphos (173 mg, 0.30 mmol), Cs₂CO₃ (1.54 g, 4.72 mmol) at 120° C. for 12 h. The reaction mixture was filtered through Celite® and the filter cake was washed with DCM/MeOH (10/1, 100 mL). The filtrate was concentrated in vacuo to give the crude, which was purified by column chromatography (eluent: DCM/MeOH=DCM/MeOH=0˜5%) to give 1-(2-(((tert-butyldimethylsilyl)oxy)methyl)-6-cyclopropylimidazo[1,2-a]pyridin-8-yl)-3-(oxetan-3-yl)imidazolidine-2,4-dione (400 mg, yield 56%) as a yellow solid. ESI-MS [M+H]+: 457.2.

### Synthesis of 1-(6-cyclopropyl-2-(hydroxymethyl)imidazo[1,2-a]pyridin-8-yl)-3-(oxetan-3-yl)imidazolidine-2,4-dione

To a solution of 1-(2-(((tert-butyldimethylsilyl)oxy)methyl)-6-cyclopropylimidazo[1,2-a]pyridin-8-yl)-3-(oxetan-3-yl)imidazolidine-2,4-dione (280 mg, 0.61 mmol) in DCM (10 mL) was added TFA (1 mL) and the mixture was stirred at room temperature for 2 h. The reaction mixture was quenched with NaHCO₃ (sat. aq., 50 mL) and extracted with EtOAc (30 mL×3). The combined organic layers were washed with brine (30 mL), dried over Na₂SO₄ and concentrated in vacuo to give 1-(6-cyclopropyl-2-(hydroxymethyl)imidazo[1,2-a]pyridin-8-yl)-3-(oxetan-3-yl)imidazolidine-2,4-dione (200 mg, 95%) as a yellow oil. ESI-MS [M+H]+: 343.2.

### Synthesis of 6-cyclopropyl-8-(3-(oxetan-3-yl)-2,4-dioxoimidazolidin-1-yl)imidazo[1,2-a]pyridine-2-carbaldehyde

A mixture of 1-(6-cyclopropyl-2-(hydroxymethyl)imidazo[1,2-a]pyridin-8-yl)-3-(oxetan-3-yl)imidazolidine-2,4-dione (200 mg, 0.53 mmol) and MnO₂ (452 mg, 5.3 mmol) in DCM (5 mL) was stirred at room temperature for 12 h. The reaction mixture was filtered through Celite® and the filter cake was washed with DCM/MeOH (10/1, 50 mL). The filtrate was concentrated in vacuo to give the crude, which was purified by column chromatography (eluent: EtOAc/PE=0˜30%) to give 6-cyclopropyl-8-(3-(oxetan-3-yl)-2,4-dioxoimidazolidin-1-yl)imidazo[1,2-a]pyridine-2-carbaldehyde (100 mg, 55%) as a yellow solid. ESI-MS [M+H]+: 341.2.

### Synthesis of rac-1-(6-cyclopropyl-2-(((2-((1S*,2S*)-2-(4-methylpyrimidin-2-yl)cyclopropyl)quinolin-7-yl)amino)methyl)imidazo[1,2-a]pyridin-8-yl)-3-(oxetan-3-yl)imidazolidine-2,4-dione

To a mixture of 6-cyclopropyl-8-(3-(oxetan-3-yl)-2,4-dioxoimidazolidin-1-yl)imidazo[1,2-a]pyridine-2-carbaldehyde (50 mg, 0.15 mmol) and rac-2-((1S*,2S*)-2-(4-methylpyrimidin-2-yl)cyclopropyl)quinolin-7-amine (72.0 mg, 0.24 mmol) in THF (5 mL) was added Ti(O-iPr)₄ (71 mg, 0.25 mmol) and the resulting mixture was stirred at 70° C. for 12 h. After the reaction was cooled to room temperature, NaBH₃CN (30 mg, 0.45 mmol) and MeOH (2 mL) were added and the mixture was stirred at room temperature for 1 h. Then the reaction was quenched with water (20 mL) and extracted with EtOAc (20 mL×3). The combined organic layers were washed with brine (20 mL), dried over anhydrous Na₂SO₄, and concentrated in vacuo to give the crude product, which was purified by preparative TLC (eluent: DCM/MeOH=15/1) to give rac-1-(6-cyclopropyl-2-(((2-((1S*,2S*)-2-(4-methylpyrimidin-2-yl)cyclopropyl)quinolin-7-yl)amino)methyl)imidazo[1,2-a]pyridin-8-yl)-3-(oxetan-3-yl)imidazolidine-2,4-dione (25 mg, 28%) as a yellow oil.

ESI-MS [M+H]+: 601.2, ¹H NMR (400 MHz, DMSO) δ 8.51 (d, J=5.1 Hz, 1H), 8.26 (d, J=1.0 Hz, 1H), 7.91 (d, J=8.2 Hz, 1H), 7.77 (s, 1H), 7.58 (d, J=8.9 Hz, 1H), 7.35 (d, J=1.4 Hz, 1H), 7.17 (d, J=5.1 Hz, 1H), 7.12 (d, J=8.2 Hz, 1H), 7.05 (dd, J=8.8, 2.2 Hz, 1H), 6.83-6.80 (m, 1H), 6.76 (d, J=1.8 Hz, 1H), 5.14-5.11 (m, 2H), 4.95 (s, 2H), 4.73-4.70 (m. 2H), 4.49 (d, J=5.6 Hz, 2H), 2.72-2.65 (m, 2H), 2.41 (s, 3H), 1.96-1.91 (m, 1H), 1.77-1.69 (m, 2H), 0.96-0.91 (m, 2H), 0.66-0.62 (m, 2H).

### Example 79

### Synthesis of rac-1-(6-cyclopropyl-2-(((2-((1S*,2S*)-2-(4-methylpyrimidin-2-yl)cyclopropyl)-4,4-dioxido-1H-pyrido[3,4-b][1,4]thiazin-7-yl)amino)methyl)imidazo[1,2-a]pyridin-8-yl)-3-methylimidazolidine-2,4-dione (I-79)

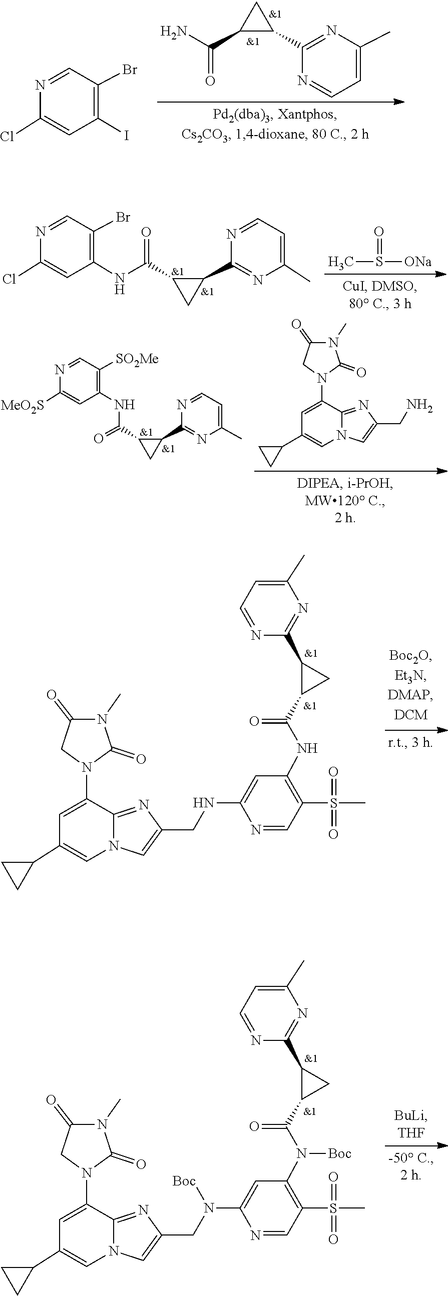

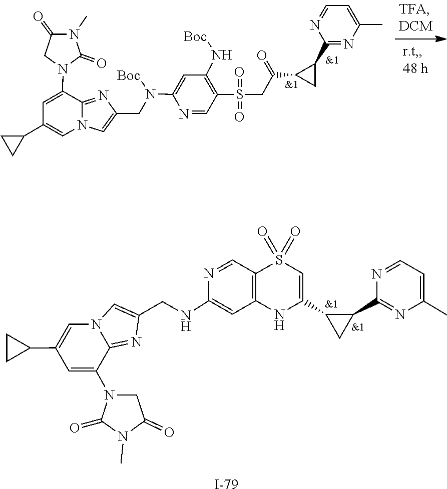

### Synthesis of rac-(1S*,2S*)—N-(5-bromo-2-chloropyridin-4-yl)-2-(4-methylpyrimidin-2-yl)cyclopropane-1-carboxamide

To a mixture of 5-bromo-2-chloro-4-iodopyridine (1 g, 3.15 mmol), rac-(1S*,2S*)-2-(4-methylpyrimidin-2-yl)cyclopropane-1-carboxamide (prepared in a similar manner to (1S,2S)-2-(4-methylpyrimidin-2-yl)cyclopropane-1-carboxamide)(558 mg, 3.15 mmol), Cs₂CO₃ (2.05 g, 6.3 mmol) in 1,4-dioxane (10 mL) was added Pd₂(dba)₃ (577 mg, 0.63 mmol) and Xantphos (365 mg, 0.63 mmol), the reaction mixture was stirred at 80° C. for 2 h under N₂. The reaction mixture was cooled to room temperature, then filtered through Celite® and the filter cake was washed with DCM/MeOH (10/1, 50 mL). The filtrate was concentrated in vacuo to give the crude, which was purified by column chromatography (eluent: PE/EtOAc=3/1) to give rac-(1S*,2S*)—N-(5-bromo-2-chloropyridin-4-yl)-2-(4-methylpyrimidin-2-yl)cyclopropane-1-carboxamide (800 mg, 69%) as a white solid. ESI-MS [M+H]+: 367.2.

### Synthesis of rac-(1S*,2S*)—N-(2,5-bis(methylsulfonyl)pyridin-4-yl)-2-(4-methylpyrimidin-2-yl)cyclopropane-1-carboxamide

To a mixture of rac-(1S*,2S*)—N-(5-bromo-2-chloropyridin-4-yl)-2-(4-methylpyrimidin-2-yl)cyclopropane-1-carboxamide (600 mg, 1.63 mmol), methanesulfinic acid sodium salt (502 mg, 4.92 mmol) in DMSO (10 mL) was added CuI (935 mg, 4.92 mmol). The mixture was stirred at 80° C. for 3 h under N₂. Water (50 mL) was added and the mixture was extracted with EtOAc (50 mL×3). The combined organic layers were washed with brine (50 mL), dried over anhydrous Na₂SO₄ and concentrated in vacuo to give the crude, which was purified by column chromatography (eluent: PE/EtOAc=1/1) to give rac-(1S*,2S*)—N-(2,5-bis(methylsulfonyl)pyridin-4-yl)-2-(4-methylpyrimidin-2-yl)cyclopropane-1-carboxamide (300 mg, 45%) as a white solid. ESI-MS [M+H]+: 411.1.

### Synthesis of rac-(1S*,2S*)—N-(2-(((6-cyclopropyl-8-(3-methyl-2,4-dioxoimidazolidin-1-yl)imidazo[1,2-a]pyridin-2-yl)methyl)amino)-5-(methylsulfonyl)pyridin-4-yl)-2-(4-methylpyrimidin-2-yl)cyclo propane-1-carboxamide

A mixture of rac-(1S*,2S*)—N-(2,5-bis(methylsulfonyl)pyridin-4-yl)-2-(4-methylpyrimidin-2-yl)cyclopropane-1-carboxamide (300 mg, 0.73 mmol), 1-(2-(aminomethyl)-6-cyclopropylimidazo[1,2-a]pyridin-8-yl)-3-methylimidazolidine-2,4-dione (284 mg, 0.95 mmol) and DIPEA (283 mg, 2.19 mmol) in i-PrOH (5 mL) was stirred in a sealed tube. After degassing with N₂ for 1 min, the reaction was irradiated in a microwave reactor at 120° C. for 2 h, the reaction was cooled to room temperature and concentrated in vacuo to give the crude, which was purified by preparative TLC (eluent: DCM/MeOH=10/1) to give rac-(1S*,2S*)—N-(2-(((6-cyclopropyl-8-(3-methyl-2,4-dioxoimidazolidin-1-yl)imidazo[1,2-a]pyridin-2-yl)methyl)amino)-5-(methylsulfonyl)pyridin-4-yl)-2-(4-methylpyrimidin-2-yl)cyclopropane-1-carboxamide (250 mg, 54%) as a white solid. ESI-MS [M+H]+: 630.2.

### Synthesis of rac-tert-butyl (4-((1S*,2S*)—N-(tert-butoxycarbonyl)-2-(4-methylpyrimidin-2-yl) cyclopropane-1-carboxamido)-5-(methylsulfonyl)pyridin-2-yl)((6-cyclopropyl-8-(3-methyl-2,4-dioxoimidazolidin-1-yl)imidazo[1,2-a]pyridin-2-yl)methyl)carbamate

To a solution of rac-(1S*,2S*)—N-(2-(((6-cyclopropyl-8-(3-methyl-2,4-dioxoimidazolidin-1-yl)imidazo[1,2-a]pyridin-2-yl)methyl)amino)-5-(methylsulfonyl)pyridin-4-yl)-2-(4-methylpyrimidin-2-yl)cyclopropane-1-carboxamide (150 mg, 0.24 mmol) in DCM (5 mL) was added di-tert-butyl dicarbonate (366 mg, 1.68 mmol), DMAP (59 mg, 0.48 mmol) and TEA (97 mg, 0.96 mmol). The mixture was stirred at room temperature for 3 h under N₂. The reaction was quenched with water (30 mL) and extracted with DCM (50 mL×3). The combined organic layers were washed with water (20 mL), dried over anhydrous Na₂SO₄, and concentrated in vacuo to give the crude product, which was purified by preparative TLC (eluent: DCM/MeOH=20/1) to give rac-tert-butyl (4-((1S*,2S*)—N-(tert-butoxycarbonyl)-2-(4-methylpyrimidin-2-yl)cyclopropane-1-carboxamido)-5-(methylsulfonyl)pyridin-2-yl)((6-cyclopropyl-8-(3-methyl-2,4-dioxoimidazolidin-1-yl)imidazo[1,2-a]pyridin-2-yl)methyl)carbamate (150 mg, 75%) as a white solid. ESI-MS [M+H]⁺: 830.3.

### Synthesis of rac-tert-butyl (4-((tert-butoxycarbonyl)amino)-5-((2-((1S*,2S*)-2-(4-methylpyrimidin-2-yl)cyclopropyl)-2-oxoethyl)sulfonyl)pyridin-2-yl)((6-cyclopropyl-8-(3-methyl-2,4-dioxoimidazolidin-1-yl)imidazo[1,2-a]pyridin-2-yl)methyl)carbamate

To a solution of rac-tert-butyl (4-((1S*,2S*)—N-(tert-butoxycarbonyl)-2-(4-methylpyrimidin-2-yl)cyclopropane-1-carboxamido)-5-(methylsulfonyl)pyridin-2-yl)((6-cyclopropyl-8-(3-methyl-2,4-dioxoimidazolidin-1-yl)imidazo[1,2-a]pyridin-2-yl)methyl)carbamate (120 mg, 0.14 mmol) in THF (5 mL) was added n-BuLi (0.3 mL, 0.72 mmol, 2.4 M in THF) dropwise at −50° C. under N₂. The mixture was stirred at −50° C. for 2 h under N₂. The mixture was quenched with NH₄Cl (sat. aq., 20 mL) and extracted with EtOAc (30 mL×3). The combined organic layers were washed with brine (20 mL), dried over anhydrous Na₂SO₄, and concentrated in vacuo to give the crude product, which was purified by preparative TLC (eluent: DCM/MeOH=10/1) to give rac-tert-butyl (4-((tert-butoxycarbonyl)amino)-5-((2-((1S*,2S*)-2-(4-methylpyrimidin-2-yl)cyclopropyl)-2-oxoethyl)sulfonyl)pyridin-2-yl)((6-cyclopropyl-8-(3-methyl-2,4-dioxoimidazolidin-1-yl)imidazo[1,2-a]pyridin-2-yl)methyl)carbamate (50 mg, 42%) as a white solid. ESI-MS [M+H]⁺: 830.2

### Synthesis of rac-1-(6-cyclopropyl-2-(((2-((1S*,2S*)-2-(4-methylpyrimidin-2-yl)cyclopropyl)-4,4-dioxido-1H-pyrido[3,4-b][1,4]thiazin-7-yl)amino)methyl)imidazo[1,2-a]pyridin-8-yl)-3-methylimidazolidine-2,4-dione

To a mixture of tert-butyl (4-((tert-butoxycarbonyl)amino)-5-((2-(2-(4-methylpyrimidin-2-yl)cyclopropyl)-2-oxoethyl)sulfonyl)pyridin-2-yl)((6-cyclopropyl-8-(3-methyl-2,4-dioxoimidazolidin-1-yl)imidazo[1,2-a]pyridin-2-yl)methyl)carbamate (40 mg, 0.048 mmol) in DCM (5 mL) was added TFA (1 mL). The mixture was stirred at room temperature for 48 h under N₂. The mixture was concentrated in vacuo, the residue was neutralized with NaHCO₃ (sat. aq., 20 mL) and extracted with DCM (30 mL×3). The combined organic layers were washed with brine (30 mL), dried over anhydrous Na₂SO₄ and concentrated in vacuo to give the crude, which was purified by preparative TLC (eluent: DCM/MeOH=10/1) to give rac-1-(6-cyclopropyl-2-(((2-((1S*,2S*)-2-(4-methylpyrimidin-2-yl)cyclopropyl)-4,4-dioxido-1H-pyrido[3,4-b][1,4]thiazin-7-yl)amino)methyl)imidazo[1,2-a]pyridin-8-yl)-3-methylimidazolidine-2,4-dione (10 mg, 34%) as a white solid.

ESI-MS [M+H]⁺: 612.2, ¹H NMR (400 MHz, DMSO) δ 10.50 (s, 1H), 8.53 (d, J=5.1 Hz, 1H), 8.42 (s, 1H), 8.23 (d, J=1.0 Hz, 1H), 7.69-7.67 (m, 2H), 7.33 (d, J=1.4 Hz, 1H), 7.21 (d, J=5.1 Hz, 1H), 6.15 (s, 1H), 5.95 (s, 1H), 4.89 (s, 2H), 4.54 (d, J=5.3 Hz, 2H), 2.97 (s, 3H), 2.54-2.53 (m, 1H), 2.42 (s, 3H), 2.34-2.29 (m, 1H), 1.99-1.89 (m, 1H), 1.74-1.64 (m, 1H), 1.57-1.55 (m, 1H), 0.96-0.91 (m, 2H), 0.68-0.61 (m, 2H).

### Example 80

### Synthesis of rac-6-(((6-cyclopropylimidazo[1,2-a]pyridin-2-yl)methyl)(methyl)amino)-3-((1S*,2S*)-2-(4-methylpyrimidin-2-yl)cyclopropyl)-4H-benzo[e][1,2,4]thiadiazine 1,1-dioxide (I-80)

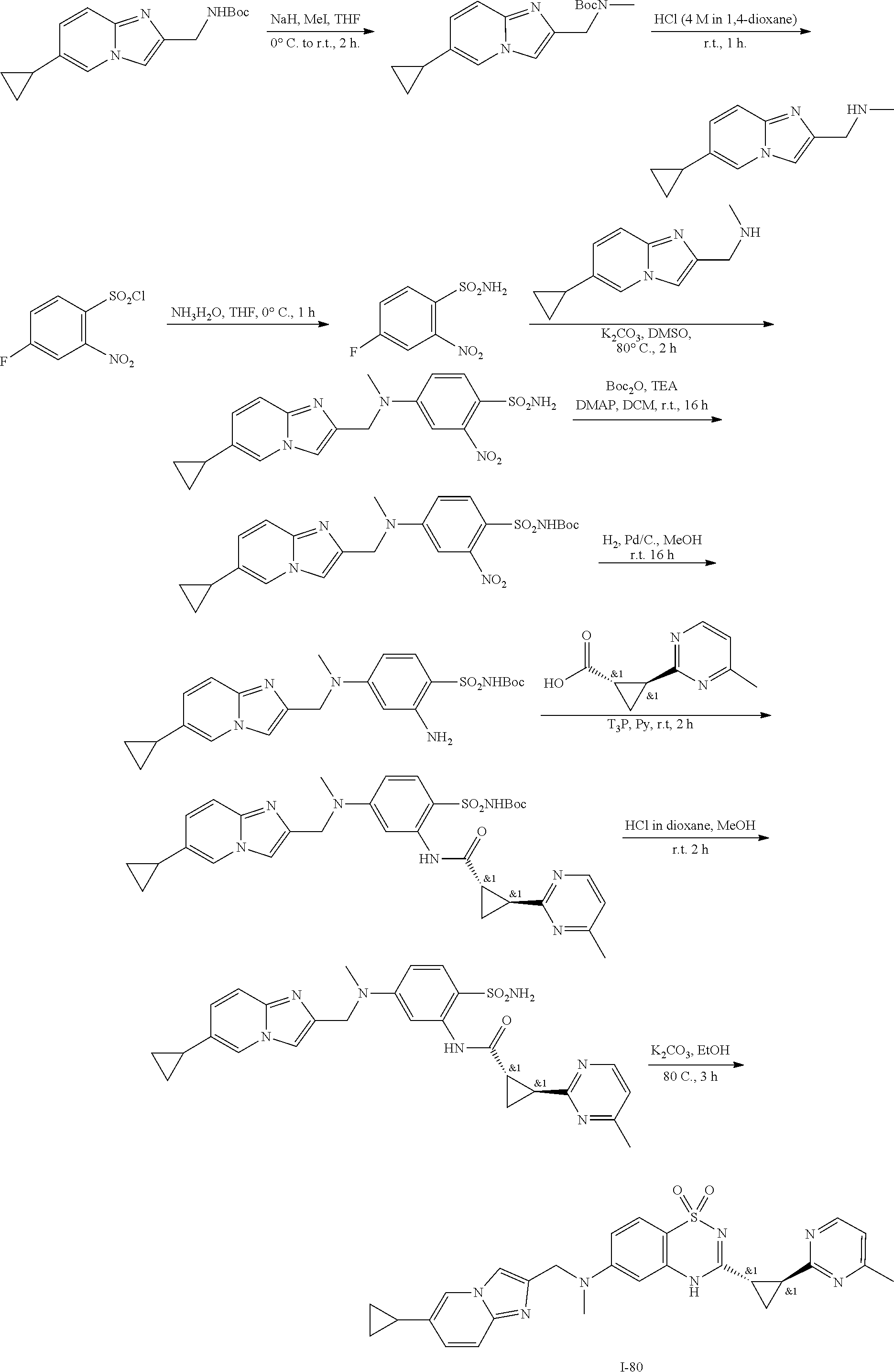

### Synthesis of tert-butyl ((6-cyclopropylimidazo[1,2-a]pyridin-2-yl)methyl)(methyl)carbamate

To a solution of tert-butyl ((6-cyclopropylimidazo[1,2-a]pyridin-2-yl)methyl)carbamate (2.90 g, 10.1 mmol) in THF (30 mL) was added NaH (60% in mineral oil, 808 mg, 20.2 mmol) at 0° C. The reaction mixture was stirred at 0° C. for 0.5 h under N₂ and then a solution of MeI (1.43 g, 10.1 mmol) in THF (10 mL) was added. The mixture was stirred at room temperature for 2 h. The reaction was quenched with water (50 mL) and extracted with EtOAc (30 mL×3). The combined organic layers were washed with brine (50 mL), dried over anhydrous Na₂SO₄, and concentrated in vacuo to give the crude product, which was purified by column chromatography (eluent: DCM/MeOH=40/1) to give tert-butyl ((6-cyclopropylimidazo[1,2-a]pyridin-2-yl)methyl)(methyl)carbamate (2.7 g, 89%) as a white solid. ESI-MS [M+H]⁺: 302.2.

### Synthesis of 1-(6-cyclopropylimidazo[1,2-a]pyridin-2-yl)-N-methylmethanamine

A mixture of tert-butyl ((6-cyclopropylimidazo[1,2-a]pyridin-2-yl)methyl)(methyl)carbamate (2.7 g, 9.0 mmol) in HCl (4M solution in 1,4-dioxane, 20 mL) was stirred at room temperature for 1 h. The mixture was quenched with NaHCO₃ (sat. aq., 60 mL) and extracted with DCM (30 mL×3). The combined organic layers were washed with brine (50 mL), dried over anhydrous Na₂SO₄, and concentrated in vacuo to give the crude product, which was purified by column chromatography (eluent: DCM/MeOH=20/1) to give 1-(6-cyclopropylimidazo[1,2-a]pyridin-2-yl)-N-methylmethanamine (1.6 g, 88%) as a yellow solid. ESI-MS [M+H]+: 202.2.

### Synthesis of 4-fluoro-2-nitrobenzenesulfonamide

To a solution of 4-fluoro-2-nitrobenzenesulfonyl chloride (1 g, 4.2 mmol) in THF (10 mL) was added NH₄OH (1 mL, conc.) at 0° C. The mixture was stirred at 0° C. for 1 h and concentrated in vacuo to give 4-fluoro-2-nitrobenzenesulfonamide (920 mg, quant) as a yellow solid. ESI-MS [M+H]+: 221.0.

### Synthesis of 4-(((6-cyclopropylimidazo[1,2-a]pyridin-2-yl)methyl)(methyl)amino)-2-nitrobenzenesulfonamide

A mixture of 4-fluoro-2-nitrobenzenesulfonamide (800 mg, 3.64 mmol), 1-(6-cyclopropylimidazo[1,2-a]pyridin-2-yl)-N-methylmethanamine hydrochloride (949 mg, 4 mmol), and K₂CO₃ (1.26 g, 9.1 mmol) in DMSO (10 mL) was stirred at 85° C. for 2 h under N₂, The reaction was cooled to room temperature then quenched with water (50 mL) and extracted with EtOAc (30 mL×3). The combined organic layers were washed with brine (50 mL), dried over anhydrous Na₂SO₄, and concentrated in vacuo to give the crude product, which was purified by column chromatography (eluent: DCM/MeOH=30/1) to give 4-(((6-cyclopropylimidazo[1,2-a]pyridin-2-yl)methyl)(methyl) amino)-2-nitrobenzenesulfonamide (600 mg, 41%) as a yellow solid. ESI-MS [M+H]+: 402.1.

### Synthesis of tert-butyl ((4-(((6-cyclopropylimidazo[1,2-a]pyridin-2-yl)methyl)(methyl)amino)-2-nitrophenyl)sulfonyl)carbamate

To a solution of 4-(((6-cyclopropylimidazo[1,2-a]pyridin-2-yl)methyl)(methyl)amino)-2-nitrobenzenesulfonamide (600 mg, 1.5 mmol) in DCM (15 mL) was added di-tert-butyl dicarbonate (562 mg, 2.58 mmol), DMAP (315 mg, 2.58 mmol) and TEA (391 mg, 3.87 mmol). The mixture was stirred at room temperature for 16 h under N₂. Water (30 mL) was added and the mixture was extracted with DCM (30 mL×3). The combined organic layers were washed with water (20 mL), dried over anhydrous Na₂SO₄, and concentrated in vacuo to give the crude product, which was purified by column chromatography (eluent: DCM/MeOH=30/1) to give tert-butyl (4-(N-(tert-butoxycarbonyl)sulfamoyl)-3-nitrophenyl)((6-cyclopropylimidazo[1,2-a]pyridin-2-yl)methyl)carbamate (300 mg, 40%) as a yellow solid. ESI-MS [M+H]⁺: 502.2.

### Synthesis of tert-butyl ((2-amino-4-(((6-cyclopropylimidazo[1,2-a]pyridin-2-yl)methyl)(methyl) amino)phenyl)sulfonyl)carbamate

To a mixture of tert-butyl ((4-(((6-cyclopropylimidazo[1,2-a]pyridin-2-yl)methyl)(methyl)amino)-2-nitrophenyl)sulfonyl)carbamate (200 mg, 0.4 mmol) in MeOH (5 mL) was added Pd/C (50 mg). The mixture was stirred at room temperature for 16 h under H₂. The reaction mixture was filtered through Celite® and the filter cake was washed with DCM/MeOH (10/1, 50 mL). The filtrate was concentrated in vacuo to give the crude, which was purified by purified by preparative TLC (eluent: DCM/MeOH=10/1) to give tert-butyl ((2-amino-4-(((6-cyclopropylimidazo[1,2-a]pyridin-2-yl)methyl)(methyl) amino)phenyl) sulfonyl)carbamate (100 mg, 53%) as a yellow solid. ESI-MS [M+H]+: 472.2.

### Synthesis of rac-tert-butyl ((4-(((6-cyclopropylimidazo[1,2-a]pyridin-2-yl)methyl)(methyl)amino)-2-((1S*,2S*)-2-(4-methylpyrimidin-2-yl)cyclopropane-1-carboxamido)phenyl)sulfonyl)carbamate

To a mixture of tert-butyl ((2-amino-4-(((6-cyclopropylimidazo[1,2-a]pyridin-2-yl)methyl)(methyl)amino)phenyl)sulfonyl)carbamate (100 mg, 0.21 mmol), rac-(1S*,2S*)-2-(4-methylpyrimidin-2-yl)cyclopropane-1-carboxylic acid (38 mg, 0.21 mmol) in pyridine (3 mL) was added T₃P (1 g, 1.57 mmol, 50% in EtOAc) dropwise at room temperature. The mixture was stirred at room temperature for 2 h. Water (50 mL) was added and the mixture was extracted with EtOAc (30 mL×3). The combined organic layers were washed with brine (50 mL), dried over anhydrous Na₂SO₄, and concentrated in vacuo to give the crude, which was purified by preparative TLC (eluent: DCM/MeOH=10/1) to give rac-tert-butyl ((4-(((6-cyclopropylimidazo[1,2-a]pyridin-2-yl)methyl)(methyl)amino)-2-((1S*,2S*)-2-(4-methylpyrimidin-2-yl)cyclopropane-1-carboxamido)phenyl)sulfonyl)carbamate (100 mg, 75%) as a yellow solid. ESI-MS [M+H]+: 632.3.

### Synthesis of rac-(1S*,2S*)—N-(5-(((6-cyclopropylimidazo[1,2-a]pyridin-2-yl)methyl)(methyl)amino)-2-sulfamoylphenyl)-2-(4-methylpyrimidin-2-yl)cyclopropane-1-carboxamide

To a solution of rac-tert-butyl ((4-(((6-cyclopropylimidazo[1,2-a]pyridin-2-yl)methyl) (methyl)amino)-2-((1S*,2S*)-2-(4-methylpyrimidin-2-yl)cyclopropane-1-carboxamido)phenyl) sulfonyl)carbamate (100 mg, 0.16 mmol) in MeOH (3 mL) was added HCl (4M solution in dioxane, 2 mL). The resulting mixture was stirred at room temperature for 2 h. The reaction mixture was concentrated in vacuo to give rac-(1S*,2S*)—N-(5-(((6-cyclopropylimidazo[1,2-a]pyridin-2-yl)methyl) (methyl)amino)-2-sulfamoylphenyl)-2-(4-methylpyrimidin-2-yl)cyclopropane-1-carboxamide (84 mg, quant) as a yellow solid, which was used in the next step without purification. ESI-MS [M+H]⁺: 532.2

### Synthesis of rac-6-(((6-cyclopropylimidazo[1,2-a]pyridin-2-yl)methyl)(methyl)amino)-3-((1S*,2S*)-2-(4-methylpyrimidin-2-yl)cyclopropyl)-4H-benzo[e][1,2,4]thiadiazine 1,1-dioxide

To a solution of rac-(1S*,2S*)—N-(5-(((6-cyclopropylimidazo[1,2-a]pyridin-2-yl)methyl)(methyl)amino)-2-sulfamoylphenyl)-2-(4-methylpyrimidin-2-yl)cyclopropane-1-carboxamide (84 mg, 0.16 mmol) in EtOH (3 mL) was added K₂CO₃ (110 mg, 0.8 mmol). The mixture was stirred at 80° C. for 3 h. The reaction mixture was cooled to room temperature, filtered through Celite® and the filter cake was washed with DCM/MeOH (10/1, 100 mL). The filtrate was concentrated in vacuo to give the crude, which was purified by preparative TLC (eluent: DCM/MeOH=10/1) to give rac-6-(((6-cyclo propylimidazo[1,2-a]pyridin-2-yl)methyl)(methyl)amino)-3-((1S*,2S*)-2-(4-methylpyrimidin-2-yl)cyclopropyl)-4H-benzo[e][1,2,4]thiadiazine 1,1-dioxide (40 mg, 49%) as a white solid.

ESI-MS [M+H]⁺: 514.3. Purity: 96.74 (214 nm), 96.26 (254 nm), ¹H NMR (400 MHz, DMSO) δ 12.03 (s, 1H), 8.55 (d, J=5.1 Hz, 1H), 8.25 (s, 1H), 7.56 (s, 1H), 7.49 (d, J=9.0 Hz, 1H), 7.37 (d, J=9.3 Hz, 1H), 7.24 (d, J=5.1 Hz, 1H), 6.96 (dd, J=9.3, 1.7 Hz, 1H), 6.90 (dd, J=9.1, 2.3 Hz, 1H), 6.41 (d, J=1.9 Hz, 1H), 4.67 (s, 2H), 3.12 (s, 3H), 2.67-2.64 (m, 1H), 2.47-2.44 (m, 1H), 2.43 (s, 3H), 1.89-1.88 (m, 1H), 1.69-1.66 (m, 2H), 0.91-0.86 (m, 2H), 0.67-0.62 (m, 2H).

### Example 81

### Synthesis of rac-1-(6-cyclopropyl-2-(((2-((1S*,2S*)-2-(4-methylpyrimidin-2-yl)cyclopropyl)quinolin-7-yl)amino)methyl)imidazo[1,2-a]pyridin-8-yl)imidazolidine-2,4-dione (I-81)

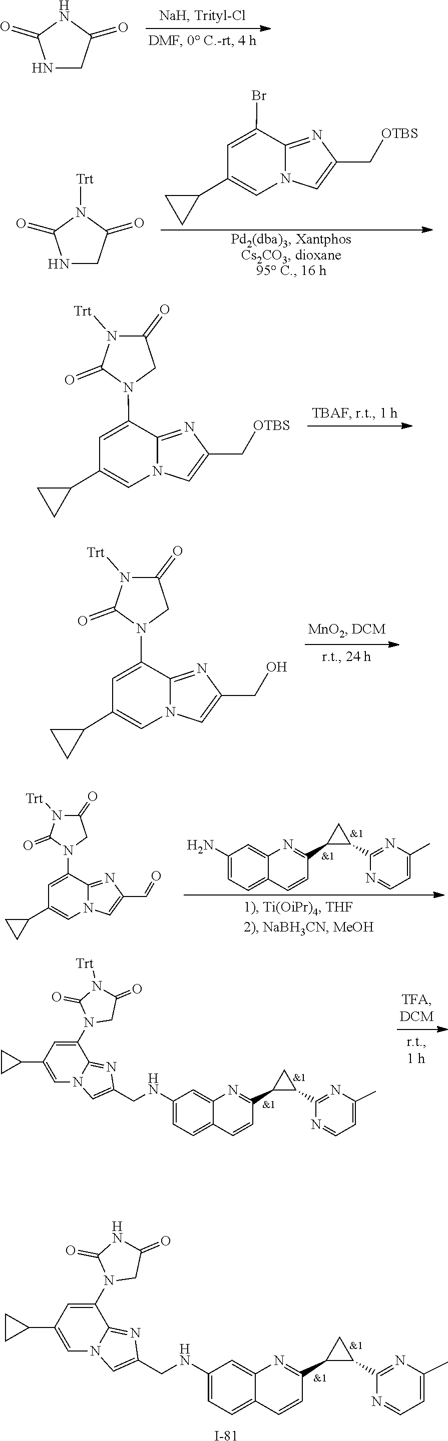

### Synthesis of 3-tritylimidazolidine-2,4-dione

To a mixture of imidazolidine-2,4-dione (100 mg, 1.0 mmol) in DMF (4 mL) was added NaH (60 mg, 60% dispersion in mineral oil, 1.50 mmol) under N₂. The resulting reaction was stirred at rt for 0.5 h, then trityl chloride (418 mg, 1.50 mmol) in DMF (1 mL) was added. The reaction mixture was stirred at rt for 4 h. The reaction was quenched with water (20 mL) and extracted with EtOAc (30 mL×3). The combined organic layers were washed with brine (10 mL), dried over anhydrous Na₂SO₄ and concentrated in vacuo to give the crude product, which was purified by column chromatography (eluent: EA/PE=1/100-100/1) to give 3-tritylimidazolidine-2,4-dione (150 mg, 44%) as a pale solid.

ESI-MS [M+H]+: 343.2.

### Synthesis of 1-(2-(((tert-butyldimethylsilyl)oxy)methyl)-6-cyclopropylimidazo[1,2-a]pyridin-8-yl)-3-tritylimidazolidine-2,4-dione

To a solution of 8-bromo-2-(((tert-butyldimethylsilyl)oxy)methyl)-6-cyclopropylimidazo[1,2-a]pyridine (494 mg, 1.3 mmol) in dioxane (10 mL) was added 3-tritylimidazolidine-2,4-dione (890 mg, 2.6 mmol), Cs₂CO₃ (1.27 g, 3.9 mmol), Xantphos (150 mg, 0.26 mmol) and Pd₂(dba)₃ (120 mg, 0.13 mmol) under N₂. The resulting mixture was stirred at 90° C. for 16 h. After the reaction was cooled to room temperature, the mixture was filtered through Celite® and the filter cake was washed with DCM/MeOH (10/1, 100 mL). The filtrate was concentrated in vacuo to give the crude, which was purified by column chromatography (eluent: EtOAc/PE from 0 to 20%) to give 1-(2-(((tert-butyldimethylsilyl)oxy)methyl)-6-cyclopropylimidazo[1,2-a]pyridin-8-yl)-3-trityl imidazolidine-2,4-dione (630 mg, 75%) as a white solid. ESI-MS [M+H]+: 643.3.

### Synthesis of 1-(6-cyclopropyl-2-(hydroxymethyl)imidazo[1,2-a]pyridin-8-yl)-3-tritylimidazolidine-2,4-dione

To a solution of 1-(2-(((tert-butyldimethylsilyl)oxy)methyl)-6-cyclopropylimidazo[1,2-a]pyridin-8-yl)-3-tritylimidazolidine-2,4-dione (630 mg, 1.0 mmol) in THF (5 mL) was added TBAF (2 mL, 2.0 mmol, 1M in THF) and the mixture was stirred at room temperature for 1 h. Water (50 mL) was added and extracted with EtOAc (30 mL×3). The combined organic layers were washed with brine (30 mL), dried over Na₂SO₄ and concentrated in vacuo. The residue was purified by flash column chromatography (eluent: EtOAc/PE from 0 to 50%) to give 1-(6-cyclopropyl-2-(hydroxymethyl) imidazo[1,2-a]pyridin-8-yl)-3-tritylimidazolidine-2,4-dione (270 mg, 51%) as a white solid. ESI-MS [M+H]+: 529.2.

### Synthesis of 6-cyclopropyl-8-(2,4-dioxo-3-tritylimidazolidin-1-yl)imidazo[1,2-a]pyridine-2-carbaldehyde

A mixture of 1-(6-cyclopropyl-2-(hydroxymethyl)imidazo[1,2-a]pyridin-8-yl)-3-tritylimidazolidine-2,4-dione (270 mg, 0.51 mmol) and MnO₂ (890 mg, 10 mmol) in DCM (10 mL) was stirred at room temperature for 24 h. The reaction mixture was filtered through Celite® and the filter cake was washed with DCM/MeOH (10/1, 50 mL). The filtrate was concentrated in vacuo to give the crude, which was purified by column chromatography (eluent: EtOAc/PE from 0 to 30%) to give 6-cyclopropyl-8-(2,4-dioxo-3-tritylimidazolidin-1-yl)imidazo[1,2-a]pyridine-2-carbaldehyde (190 mg, 70%) as a white solid. ESI-MS [M+H]+: 527.2

### Synthesis of rac-1-(6-cyclopropyl-2-(((2-((1S*,2S*)-2-(4-methylpyrimidin-2-yl)cyclopropyl)quinolin-7-yl)amino)methyl)imidazo[1,2-a]pyridin-8-yl)-3-tritylimidazolidine-2,4-dione

To a mixture of 6-cyclopropyl-8-(2,4-dioxo-3-tritylimidazolidin-1-yl)imidazo[1,2-a]pyridine-2-carbaldehyde (190 mg, 0.36 mmol) and rac-2-((1S*,2S*)-2-(4-methylpyrimidin-2-yl)cyclopropyl)quinolin-7-amine (100 mg, 0.36 mmol) in THF (20 mL) was added Ti(OiPr)₄ (511 mg, 1.8 mmol) at room temperature under nitrogen and the mixture was stirred at 70° C. for 16 h. After the reaction was cooled to room temperature, MeOH (2 mL) was added, followed by NaBH₃CN (113 mg, 1.8 mmol) and the mixture was stirred at room temperature for 1 h. The mixture was quenched with NH₄Cl (sat. aq., 50 mL) and extracted with EtOAc (30 mL×3). The combined organic layers were washed with brine (30 mL), dried over Na₂SO₄ and concentrated in vacuo. The residue was purified by flash column chromatography (eluent: MeOH:DCM=0-5%) to give rac-1-(6-cyclopropyl-2-(((2-((1S*,2S*)-2-(4-methylpyrimidin-2-yl)cyclopropyl)quinolin-7-yl)amino)methyl)imidazo[1,2-a]pyridin-8-yl)-3-tritylimidazolidine-2,4-dione (230 mg, 81%) as a yellow solid. ESI-MS [M+H]+: 787.3.

### Synthesis of rac-1-(6-cyclopropyl-2-(((2-((1S*,2S*)-2-(4-methylpyrimidin-2-yl)cyclopropyl)quinolin-7-yl)amino)methyl)imidazo[1,2-a]pyridin-8-yl)imidazolidine-2,4-dione

To a mixture of rac-1-(6-cyclopropyl-2-(((2-((1S*,2S*)-2-(4-methylpyrimidin-2-yl)cyclopropyl)quinolin-7-yl)amino)methyl)imidazo[1,2-a]pyridin-8-yl)-3-tritylimidazolidine-2,4-dione (130 mg, 0.16 mmol) in DCM (10 mL) was added TFA (2 mL), the reaction mixture was stirred at room temperature for 1 h. The reaction mixture was quenched with NaHCO₃ (sat. aq., 50 mL) and extracted with extracted with EtOAc (30 mL×3). The combined organic layers were washed with brine (30 mL), dried over Na₂SO₄ and concentrated in vacuo. The residue was purified by preparative TLC (eluted: DCM:MeOH=10:1) to give rac-1-(6-cyclopropyl-2-(((2-((1S*,2S*)-2-(4-methylpyrimidin-2-yl)cyclopropyl)quinolin-7-yl)amino)methyl)imidazo[1,2-a]pyridin-8-yl)imidazolidine-2,4-dione (60 mg, 68%) as a yellow solid.

ESI-MS [M+H]⁺: 545.2. ¹H NMR (400 MHz, DMSO) δ 11.34 (s, 1H), 8.51 (d, J=5.1 Hz, 1H), 8.23 (d, J=1.1 Hz, 1H), 7.91 (d, J=8.2 Hz, 1H), 7.75 (s, 1H), 7.57 (d, J=8.9 Hz, 1H), 7.34 (d, J=1.5 Hz, 1H), 7.17 (d, J=5.1 Hz, 1H), 7.12 (d, J=8.2 Hz, 1H), 7.06-7.04 (m, 1H), 6.82-6.79 (m, 1H), 6.77 (d, J=1.9 Hz, 1H), 4.91 (s, 2H), 4.48 (d, J=5.7 Hz, 2H), 2.74-2.63 (m, 2H), 2.41 (s, 3H), 1.95-1.89 (m, 1H), 1.78-1.69 (m, 2H), 0.94-0.89 (m, 2H), 0.68-0.59 (m, 2H).

### Example 82

### Synthesis rac-7-(((6-cyclopropyl-8-(3-methyl-2,4-dioxoimidazolidin-1-yl)imidazo[1,2-a]pyridin-2-yl)methyl)amino)-2-((1S*,2S*)-2-(4-methylpyrimidin-2-yl)cyclopropyl)quinoline-5-carbonitrile (I-82)

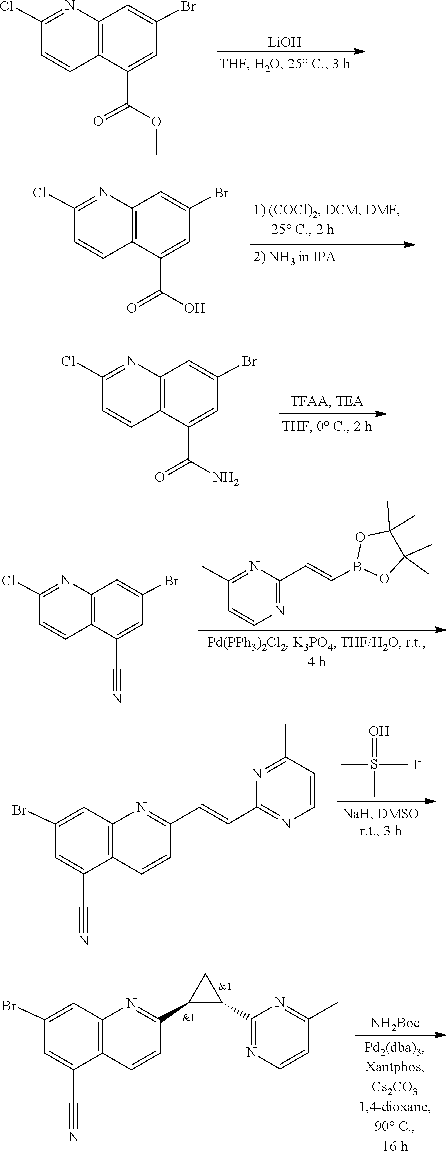

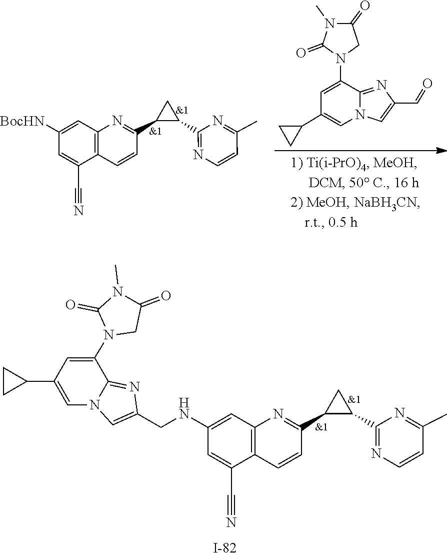

### Synthesis of 7-bromo-2-chloroquinoline-5-carboxylic acid

A mixture of methyl 7-bromo-2-chloroquinoline-5-carboxylate (1.8 g, 6.0 mmol) and LiOH—H₂O (1.26 g, 30.0 mmol) in THF/H₂O (25 mL/25 mL) was stirred at 25° C. for 3 h. The reaction mixture was cooled to room temperature and was acidified with HCl (4M in water, 10 mL), extracted with DCM/MeOH (10/1, 100 mL×3). The combined organic layers were washed with brine (50 mL), dried over anhydrous Na₂SO₄, and concentrated in vacuo to give the crude, which was purified by column chromatography (eluent: DCM/MeOH=1/1) to give 7-bromo-2-chloroquinoline-5-carboxylic acid (1.5 g, 87%) as a yellow solid. ESI-MS [M+H]+: 285.9.

### Synthesis of 7-bromo-2-chloroquinoline-5-carboxamide

A mixture of 7-bromo-2-chloroquinoline-5-carboxylic acid (1.5 g, 5.3 mmol), (COCl)₂ (1.33 g, 11.0 mmol) and DMF (0.1 mL) in DCM (15 mL) was stirred at 25° C. for 3 h. The reaction mixture was concentrated, and the residue was neutralized with NH₃ (2M in i-PrOH solution, 20 mL). The reaction mixture was stirred at 25° C. for 1 h then concentrated in vacuo to give the crude, which was purified by column chromatography (eluent: DCM/MeOH=10/1) to give 7-bromo-2-chloroquinoline-5-carboxamide (1.4 g, 92%) as a white solid. ESI-MS [M+H]+: 284.9.

### Synthesis of 7-bromo-2-chloroquinoline-5-carbonitrile

A mixture of 17-bromo-2-chloroquinoline-5-carboxamide (1.4 g, 4.9 mmol), TFAA (3.5 mL) and TEA (7.2 mL) in THF (30 mL) was stirred at 0° C. for 2 h under N₂. The reaction was quenched with water (50 mL) and extracted with EtOAc (50 mL×3). The combined organic layers were washed with brine (50 mL), dried over anhydrous Na₂SO₄, and concentrated in vacuo to give the crude, which was purified by column chromatography (eluent: PE/EtOAc=2/1) to give 7-bromo-2-chloroquinoline-5-carbonitrile (1.07 g, 82%) as a white solid. ESI-MS [M+H]+: 266.9.

### Synthesis of: 7-bromo-2-chloroquinoline-5-carbonitrile

A mixture of 7-bromo-2-chloroquinoline-5-carbonitrile (1.07 g, 4.0 mmol), (E)-4-methyl-2-(2-(4,4,5,5-tetramethyl-1,3,2-dioxaborolan-2-yl)vinyl)pyrimidine (990 mg, 4.0 mmol), K₃PO₄ (2.56 g, 12.1 mmol) and Pd(PPh₃)₂Cl₂ (292 mg, 0.40 mmol) in THF/H₂O (70 mL/10 mL) was stirred at room temperature for 4 h under N₂. Water (100 mL) was added and the reaction was extracted with EtOAc (50 mL×3). The combined organic layers were washed with brine (50 mL), dried over anhydrous Na₂SO₄, and concentrated in vacuo to give the crude product, which was purified by column chromatography (eluent: PE/EtOAc=2/1) to 7-bromo-2-chloroquinoline-5-carbonitrile (800 mg, 57%) as a yellow solid. ESI-MS [M+H]⁺: 351.1

### Synthesis of rac-7-bromo-2-((1S*,2S*)-2-(4-methylpyrimidin-2-yl)cyclopropyl)quinoline-5-carbonitrile

To a suspension of trimethylsulfoxonium iodide (1.24 g, 5.63 mmol) in DMSO (20 mL) was added NaH (226 mg, 60% in mineral oil, 5.66 mmol) at 0° C. The reaction mixture was stirred at 0° C. for 1 h then a solution of (E)-7-bromo-2-(2-(4-methylpyrimidin-2-yl)vinyl)quinoline-5-carbonitrile (660 mg, 1.88 mmol) in DMSO (5 mL) was added. The resulting mixture was stirred at room temperature for 3 h under N₂. The reaction was quenched with NH₄Cl (sat. aq., 50 mL) and extracted with EtOAc (50 mL×3). The combined organic layers were washed with brine (50 mL), dried over anhydrous Na₂SO₄, and concentrated in vacuo to give the crude, which was purified by column chromatography (eluent: PE/EtOAc=2/1) to give rac-7-bromo-2-((1S*,2S*)-2-(4-methylpyrimidin-2-yl)cyclopropyl)quinoline-5-carbonitrile (250 mg, 36%) as a yellow solid. ESI-MS [M+H]+: 365.1.

### Synthesis of trac-tert-butyl (5-cyano-2-((1S*,2S*)-2-(4-methylpyrimidin-2-yl)cyclopropyl)quinolin-7-yl)carbamate

To a mixture of 7 rac-7-bromo-2-((1S*,2S*)-2-(4-methylpyrimidin-2-yl)cyclopropyl)quinoline-5-carbonitrile (70 mg, 0.19 mmol), NH₂Boc (34 mg, 0.29 mmol) and Cs₂CO₃ (190 mg, 0.58 mmol) in 1,4-dioxane (5 mL) was added Pd₂(dba)₃ (18 mg, 0.019 mmol) and XantPhos (22 mg, 0.038 mmol). The reaction mixture was stirred at 90° C. for 16 h under N₂. The reaction mixture was filtered through Celite® and the filter cake was washed with DCM/MeOH (10/1, 20 mL). The filtrate was concentrated in vacuo to give the crude, which was purified by preparative TLC (eluent: DCM/MeOH=20/1) to give rac-tert-butyl (5-cyano-2-((1S*,2S*)-2-(4-methylpyrimidin-2-yl)cyclopropyl)quinolin-7-yl)carbamate (50 mg, 66%) as a white solid. ESI-MS [M+H]+: 402.2.

### Synthesis of rac-7-amino-2-((1S*,2S*)-2-(4-methylpyrimidin-2-yl)cyclopropyl)quinoline-5-carbonitrile

To a solution of rac-tert-butyl (5-cyano-2-((1S*,2S*)-2-(4-methylpyrimidin-2-yl)cyclopropyl)quinolin-7-yl)carbamate (50 mg, 0.12 mmol) in DCM (3 mL) was added TFA (1.0 mL). The resulting mixture was stirred at room temperature for 2 h. The reaction mixture was quenched with NH₃ (7 M in MeOH, 10 mL) and concentrated in vacuo to give the crude, which was purified by preparative TLC (eluent: DCM/MeOH=20/1) to give rac-7-amino-2-((1S*,2S*)-2-(4-methylpyrimidin-2-yl)cyclopropyl)quinoline-5-carbonitrile (32 mg, 85%) as a yellow solid. ESI-MS [M+H]+: 302.2.

### Synthesis of rac-7-(((6-cyclopropyl-8-(3-methyl-2,4-dioxoimidazolidin-1-yl)imidazo[1,2-a]pyridin-2-yl)methyl)amino)-2-((1S*,2S*)-2-(4-methylpyrimidin-2-yl)cyclopropyl)quinoline-5-carbonitrile

A mixture of rac-7-amino-2-((1S*,2S*)-2-(4-methylpyrimidin-2-yl)cyclopropyl)quinoline-5-carbonitrile (30 mg, 0.10 mmol), 6-cyclopropyl-8-(3-methyl-2,4-dioxoimidazolidin-1-yl)imidazo[1,2-a]pyridine-2-carbaldehyde (30 mg, 0.10 mmol) and Ti(i-PrO)₄ (151 mg, 0.53 mmol) in DCM/MeOH (2 mL/2 mL) was stirred at 50° C. for 16 h under N₂. After cooling to 0° C., NaBH₃CN (23 mg, 0.37 mmol) was added and the mixture was stirred at room temperature for 0.5 h. Water (20 mL) was added and extracted with DCM (30 mL×3). The combined organic layers were washed with brine (30 mL), dried over anhydrous Na₂SO₄, and concentrated in vacuo to give the crude, which was purified by preparative TLC (eluent: PE/EtOAc=1/3) to give rac-7-(((6-cyclopropyl-8-(3-methyl-2,4-dioxoimidazolidin-1-yl)imidazo[1,2-a]pyridin-2-yl)methyl)amino)-2-((15S*,2S*)-2-(4-methylpyrimidin-2-yl)cyclopropyl) quinoline-5-carbonitrile (30 mg, 51%) as a white solid.

ESI-MS [M+H]+: 584.2. ¹H NMR (400 MHz, DMSO) δ=8.52 (d, J=5.1, 1H), 8.26 (d, J=1.1, 1H), 8.06 (d, J=8.5, 1H), 7.80 (s, 1H), 7.66 (d, J=2.3, 1H), 7.39-7.35 (m, 2H), 7.21-7.18 (m, 2H), 7.12 (d, J=1.9, 1H), 4.93 (s, 2H), 4.54 (d, J=5.7, 2H), 2.97 (s, 3H), 2.77-2.74 (m, 2H), 2.41 (s, 3H), 1.97-1.91 (m, 1H), 1.82-1.74 (m, 2H), 0.96-0.91 (m, 2H), 0.67-0.62 (m, 2H).

### Example 83

### Synthesis of rac-4-chloro-2-((1S*,2S*)-2-(7-(((6-cyclopropyl-8-(3-methyl-2,4-dioxoimidazolidin-1-yl)imidazo[1,2-a]pyridin-2-yl)methyl)amino)quinolin-2-yl)cyclopropyl)benzonitrile (I-83)

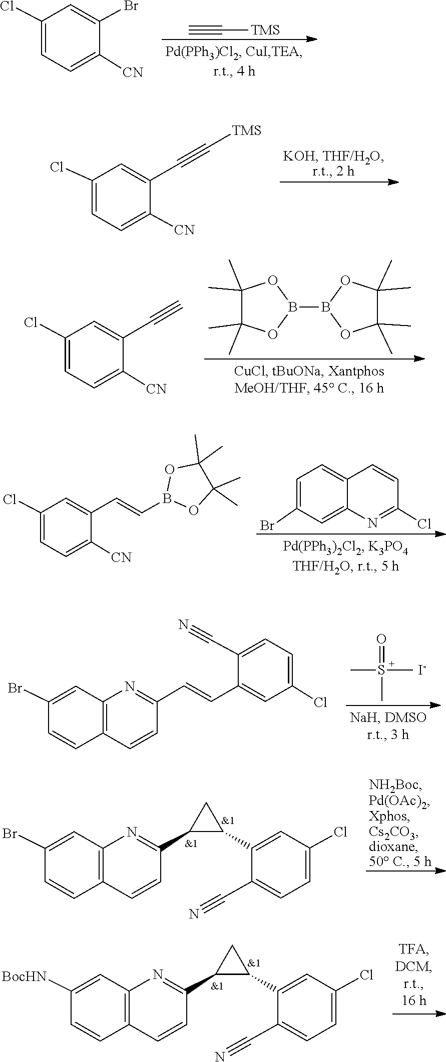

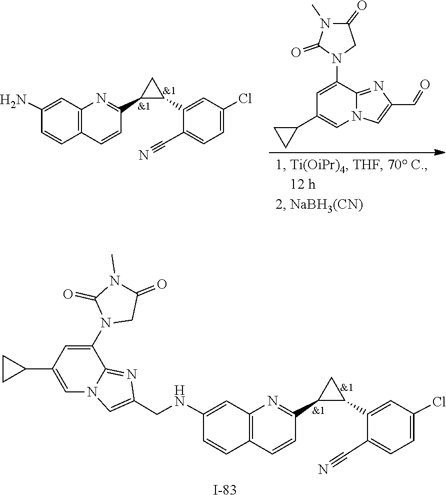

### Synthesis of 4-chloro-2-((trimethylsilyl)ethynyl)benzonitrile

To a reaction mixture of 2-bromo-4-chlorobenzonitrile (8.6 g, 40 mmol), Pd(PPh₃)₂Cl₂ (1.46 g, 2.0 mmol) and CuI (1.52 g, 8.0 mmol) in Et₃N (20.20 g, 200 mmol) was added ethynyltrimethylsilane (11.76 g, 120 mmol). The reaction was stirred at room temperature for 4 h under N₂. Water (200 mL) was added and the mixture was extracted with EtOAc (80 mL×3). The combined organic layers were washed with brine (80 mL), dried over Na₂SO₄ and concentrated in vacuo to give the crude, which was purified by column chromatography (eluent: EtOAc/PE=0˜10%) to give the 4-chloro-2-((trimethylsilyl)ethynyl)benzonitrile (6 g, 64%) as yellow solid. ESI-MS [M+H]⁺: 234.0.

### Synthesis of 4-chloro-2-ethynylbenzonitrile

To a stirred mixture of 4-chloro-2-((trimethylsilyl)ethynyl)benzonitrile (6 g, 25.7 mmol) in THF (15 ml) was added KOH solution (1 M, 60 mL). and the reaction was stirred at room temperature for 2 h. The reaction mixture was quenched with NH₄Cl (sat. aq., 100 mL) and extracted with EtOAc (50 mL×3). The combined organic layers were washed with brine (50 mL), dried over Na₂SO₄ and concentrated in vacuo to give the crude, which was purified by column chromatography (eluent: EtOAc/PE=0˜10%) to afford 4-chloro-2-ethynylbenzonitrile (3.1 g, 75%) as white solid. ESI-MS [M+H]⁺: 162.0.

### Synthesis of (E)-4-chloro-2-(2-(4,4,5,5-tetramethyl-1,3,2-dioxaborolan-2-yl)vinyl)benzonitrile

A mixture of CuCl (99 mg, 1 mmol), t-BuONa (192 mg, 2 mmol) and xantphos (579 mg, 1 mmol) in THF (50 mL) was stirred at room temperature for 0.5 h. Then 4,4,4′,4′,5,5′-hexamethyl-2,2′-bi(1,3,2-dioxaborolane) (3.8 g, 15 mmol) in THF (20 ml) was added and the mixture was stirred at room temperature for 10 min. 2-ethynyl-4-methylpyrimidine (1.61 g, 10 mmol) in THF (10 mL) and MeOH (640 mg, 20 mmol) was added successively. The resulting reaction mixture was stirred at 45° C. for 16 h. The reaction mixture was quenched with water (100 mL) and extracted with EtOAc (60 mL×3). The combined organic layers were washed with brine (60 mL), dried over Na₂SO₄ and concentrated in vacuo to give the crude, which was purified by column chromatography (eluent: EtOAc/PE=0˜10%) to give the (E)-4-chloro-2-(2-(4,4,5,5-tetramethyl-1,3,2-dioxaborolan-2-yl)vinyl)benzonitrile (860 mg, 30%) as white solid. ESI-MS [M+H]⁺: 290.1.

### Synthesis of (E)-2-(2-(7-bromoquinolin-2-yl)vinyl)-4-chlorobenzonitrile

To a mixture of (E)-4-chloro-2-(2-(4,4,5,5-tetramethyl-1,3,2-dioxaborolan-2-yl)vinyl)benzonitrile (860 mg, 3 mmol), 7-bromo-2-chloroquinoline (792 mg, 3.3 mmol) and K₃PO₄ (1.9 g, 9 mmol) in THF/H₂O (30 mL/3 mL) was added Pd(PPh₃)₂Cl₂ (233 mg, 0.3 mol). The mixture was stirred at room temperature for 16 h under N₂. The reaction mixture was quenched with water (50 mL) and extracted with EtOAc (30 mL×3). The combined organic layers were washed with brine (30 mL), dried over Na₂SO₄ and concentrated in vacuo to give the crude, which was purified by column chromatography (eluent: PE/DCM=0˜50%) to give (E)-2-(2-(7-bromoquinolin-2-yl)vinyl)-4-chlorobenzonitrile (600 mg, 55%) as a white solid. ESI-MS [M+H]+: 370.9.

### Synthesis of rac-2-((1S*,2S*)-2-(7-bromoquinolin-2-yl)cyclopropyl)-4-chlorobenzonitrile

To a stirred mixture of NaH (292 mg, 60% in mineral oil, 7.3 mmol) in DMSO (20 mL) was added trimethylsulfoxonium iodide (1.79 g, 8.1 mmol) in portions and the mixture was stirred at room temperature for 1 h under N₂. A solution of (E)-2-(2-(7-bromoquinolin-2-yl)vinyl)-4-chlorobenzonitrile (600 mg, 1.62 mmol) in DMSO (10 mL) was added and the reaction mixture was stirred at room temperature for 2 h. The final reaction mixture was quenched with water (100 mL) and extracted with EtOAc (100 mL×3). The combined organic layers were washed with brine (30 mL), dried over Na₂SO₄ and concentrated in vacuo to give the crude, which was purified by column chromatography (eluent: PE/DCM=0˜50%) to give rac-2-((1 S*,2S*)-2-(7-bromoquinolin-2-yl)cyclopropyl)-4-chlorobenzonitrile (220 mg, 35%) as a yellow solid. ESI-MS [M+H]+: 383.0

### Synthesis of rac-tert-butyl (2-((1S*,2S*)-2-(5-chloro-2-cyanophenyl)cyclopropyl)quinolin-7-yl)carbamate

To a mixture of rac-2-((1S*,2S*)-2-(7-bromoquinolin-2-yl)cyclopropyl)-4-chlorob (220 mg, 0.57 mmol), BocNH₂ (67 mg, 0.57 mmol), Xphos (52 mg, 0.11 mmol) and Cs₂CO₃ (557 mg, 1.71 mmol) in 1,4-dioxane (30 mL) was added Pd(OAc)₂ (13 mg, 0.06 mmol) and the mixture was stirred at 50° C. for 5 h under N₂. The reaction mixture was filtered through Celite® and the filter cake was washed with DCM/MeOH (10/1, 30 mL). The filtrate was concentrated in vacuo to give the crude, which was purified by column chromatography (eluent: MeOH/DCM=0˜2%) to give rac-tert-butyl (2-((1S*,2S*)-2-(5-chloro-2-cyanophenyl)cyclopropyl)quinolin-7-yl)carbamate (60 mg, 25%) as yellow solid. ESI-MS [M+H]+: 420.1.

### Synthesis of rac-2-((1S*,2S*)-2-(7-aminoquinolin-2-yl)cyclopropyl)-4-chlorobenzonitrile

To a stirred mixture of rac-tert-butyl (2-((1S*,2S*)-2-(5-chloro-2-cyanophenyl)cyclopropyl)quinolin-7-yl)carbamate (60 mg, 0.14 mol) in DCM (5 mL) was added TFA (0.5 ml). The reaction mixture was stirred at room temperature for 16 h. The reaction was quenched with NaHCO₃ (sat. aq., 30 mL) and extracted with DCM (30 mL×3). The combined organic layers were washed with brine (30 mL), dried over Na₂SO₄, concentrated in vacuo to give the crude, which was purified with preparative TLC (eluent: MeOH/DCM=1/40) to afford rac-2-((1S*,2S*)-2-(7-aminoquinolin-2-yl)cyclopropyl)-4-chlorobenzonitrile (36 mg, 79%) as a yellow solid. ESI-MS [M+H]+: 320.1.

### Synthesis of rac-4-chloro-2-((1S*,2S*)-2-(7-(((6-cyclopropyl-8-(3-methyl-2,4-dioxoimidazolidin-1-yl)imidazo[1,2-a]pyridin-2-yl)methyl)amino)quinolin-2-yl)cyclopropyl)benzonitrile

To a mixture of rac-2-((1S*,2S*)-2-(7-aminoquinolin-2-yl)cyclopropyl)-4-chlorobenzonitrile (27 mg, 0.084 mmol) and 6-cyclopropyl-8-(3-methyl-2,4-dioxoimidazolidin-1-yl)imidazo[1,2-a]pyridine-2-carbaldehyde (36 mg, 0.12 mol) in THF (15 mL) was added Ti(Oi-Pr)₄ (114 mg, 0.4 mmol). The resulting solution was stirred at 70° C. for 12 h then cooled to room temperature. MeOH (2 mL) and NaBH₃CN (15 mg, 0.24 mmol) were added and the resulting reaction mixture was stirred at room temperature for 1 h. The reaction was quenched with water (40 mL) and extracted with EtOAc (30 mL×3). The combined organic layers were washed with brine (30 mL), dried over Na₂SO₄ then concentrated in vacuo to give the crude, which was purified with preparative TLC (eluent: DCM/MeOH=20/1) to give rac-4-chloro-2-((1S*,2S*)-2-(7-(((6-cyclopropyl-8-(3-methyl-2,4-dioxoimidazolidin-1-yl)imidazo[1,2-a]pyridin-2-yl)methyl)amino)quinolin-2-yl)cyclopropyl) benzonitrile (28 mg, 55%) as yellow solid.

ESI-MS [M+H]+: 602.2, ¹H NMR (400 MHz, DMSO) δ 8.24 (d, J=1.0 Hz, 1H), 7.93 (d, J=8.2 Hz, 1H), 7.81 (d, J=8.3 Hz, 1H), 7.76 (s, 1H), 7.58 (d, J=8.9 Hz, 1H), 7.48-7.46 (m, 1H), 7.43 (d, J=1.9 Hz, 1H), 7.35 (d, J=1.4 Hz, 1H), 7.17 (d, J=8.2 Hz, 1H), 7.07-7.04 (m, 1H), 6.83-6.78 (m, 2H), 5.00-4.91 (m, 2H), 4.49 (d, J=5.6 Hz, 2H), 2.97 (s, 3H), 2.76-2.66 (m, 2H), 1.96-1.86 (m, 2H), 1.71-1.67 (m, 1H), 0.95-0.90 (m, 2H), 0.65-0.61 (m, 2H).

### Example 84

### Synthesis of rac-1-(6-cyclopropyl-2-(((6-methoxy-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (I-84)

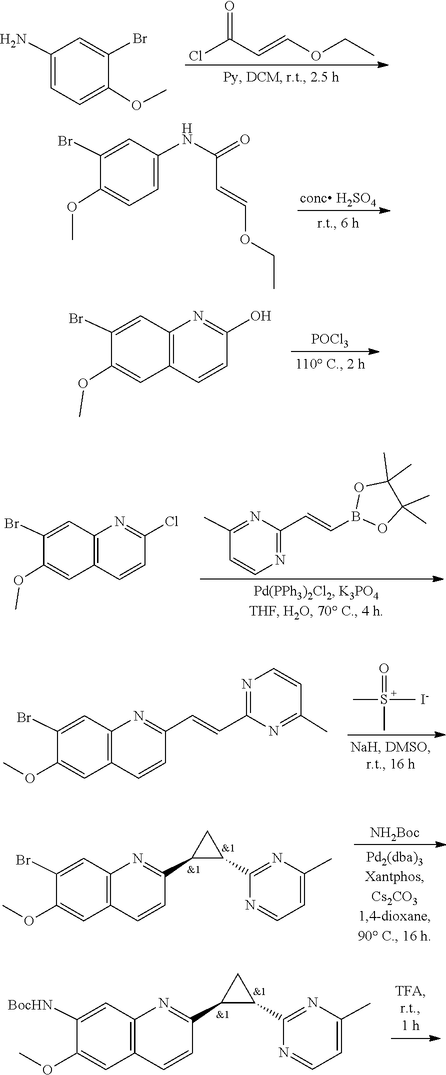

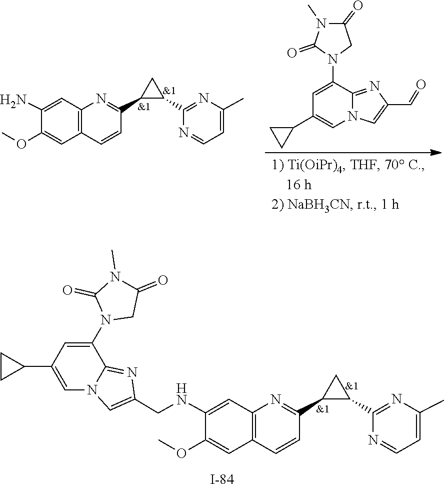

### Synthesis of (E)-N-(3-bromo-4-methoxyphenyl)-3-ethoxyacrylamide

To a mixture of 3-bromo-4-methoxy aniline (3.5 g, 17.4 mmol) and pyridine (2.7 g, 34.8 mmol) in DCM (40 mL) was added (E)-3-ethoxyacryloyl chloride (4.6 g, 34.8 mmol) in DCM (20 mL) dropwise at room temperature and the mixture was stirred at room temperature for 2.5 h. The mixture was quenched with water (50 mL) and conc. HCl (10 mL), extracted with DCM (60×3 mL). The combined organic layers were washed with water (50 mL), dried over anhydrous Na₂SO₄, and concentrated in vacuo to give the crude product, which was purified by column chromatography (eluent: PE/EtOAc=2/1) to give (E)-N-(3-bromo-4-methoxyphenyl)-3-ethoxyacrylamide (3.7 g, 71%) as a yellow solid. ESI-MS [M+H]⁺: 300.2.

### Synthesis of 7-bromo-6-methoxyquinolin-2-ol

A solution of (E)-N-(3-bromo-4-methoxyphenyl)-3-ethoxyacrylamide (3.7 g, 12.4 mmol) in con. H₂SO₄ (30 mL) was stirred at room temperature for 6 h. The mixture was poured into ice water (100 mL) slowly with stirring and adjusted pH=7-8 with Na₂CO₃ (sat. aq.). The mixture was extracted with DCM (100×3 mL). The combined organic layers were washed with brine (50 mL), dried over anhydrous Na₂SO₄ and concentrated in vacuo to give 7-bromo-6-methoxyquinolin-2-ol (3.3 g, crude) as a yellow solid, which was used directly into the next step without further purification. ESI-MS [M+H]⁺: 254.0.

### Synthesis of 7-bromo-2-chloro-6-methoxyquinoline

A mixture of 7-bromo-6-methoxyquinolin-2-ol (6.6 g, crude) in POCl₃ (50 mL) was stirred at 110° C. for 2 h. After cooling to room temperature, the mixture was concentrated in vacuo and the residue was poured into ice water (100 mL) slowly. The mixture was extracted with DCM (100×3 mL). The combined organic layers were washed with brine (20 mL), dried over anhydrous Na₂SO₄ and concentrated in vacuo to give the crude product, which was purified by column chromatography (eluent: PE/EtOAc=2/1) to give 7-bromo-2-chloro-6-methoxyquinoline (3.6 g, 54%) as a white solid. ESI-MS [M+H]⁺: 271.9.

### Synthesis of (E)-7-bromo-6-methoxy-2-(2-(4-methylpyrimidin-2-yl)vinyl)quinoline

A mixture of 7-bromo-2-chloro-6-methoxyquinoline (3.6 g, 13.2 mmol), (E)-4-methyl-2-(2-(4,4,5,5-tetramethyl-1,3,2-dioxaborolan-2-yl)vinyl)pyrimidine (3.26 g, 13.2 mmol) and K₃PO₄ (8.4 g, 39.6 mmol) in THF/H₂O (50/5 mL) was added Pd(PPh₃)₂Cl₂ (925 mg, 1.32 mmol) at room temperature. The mixture was stirred at 70° C. for 4 h under N₂. After cooling to room temperature, water (50 mL) was added and the mixture was extracted with EtOAc (50 mL×3). The combined organic layers were washed with brine (60 mL), dried over anhydrous Na₂SO₄, and concentrated in vacuo to give the crude, which was purified by column chromatography (eluent: PE/EtOAc=1/1) to give (E)-7-bromo-6-methoxy-2-(2-(4-methylpyrimidin-2-yl)vinyl)quinoline (950 mg, 20%) as a yellow solid. ESI-MS [M+H]⁺: 356.2.

### Synthesis of rac-7-bromo-6-methoxy-2-((1S*,2S*)-2-(4-methylpyrimidin-2-yl)cyclopropyl)quinoline

A suspension of trimethylsulfoxonium iodide (1.68 g, 7.65 mmol) in DMSO (20 mL) was added NaH (306 mg, 60% in mineral oil, 7.65 mmol) at 0° C. The mixture was stirred at room temperature for 1 h. Then a solution of (E)-7-bromo-6-methoxy-2-(2-(4-methylpyrimidin-2-yl)vinyl)quinoline (910 mg, 2.55 mmol) in DMSO (5 mL) was added and the mixture was stirred at room temperature for 16 h. The mixture was quenched with NH₄Cl (sat. aq., 100 mL) and extracted with EtOAc (100×3 mL). The combined organic layers were washed with brine (50 mL), dried over anhydrous Na₂SO₄, and concentrated in vacuo to give the crude product, which was purified by column chromatography (eluent: PE/EtOAc=3/1) to give rac-7-bromo-6-methoxy-2-((1S*,2S*)-2-(4-methylpyrimidin-2-yl)cyclopropyl)quinoline (462 mg, 49%) as a yellow oil. ESI-MS [M+H]⁺: 370.1.

### Synthesis of rac-tert-butyl (6-methoxy-2-((1S*,2S*)-2-(4-methylpyrimidin-2-yl)cyclopropyl)quinolin-7-yl)carbamate

To a mixture of rac-7-bromo-6-methoxy-2-((1S*,2S*)-2-(4-methylpyrimidin-2-yl)cyclopropyl)quinoline (229 mg, 0.62 mmol), tert-butyl carbamate (109 mg, 0.93 mmol) and Cs₂CO₃ (610 mg, 1.86 mmol) in 1,4-dioxane (15 mL) was added Pd₂(dba)₃ (57 mg, 0.062 mmol) and Xantphos (69 mg, 0.12 mmol) at room temperature. The mixture was stirred at 90° C. for 16 h under N₂. After cooling to room temperature, the reaction mixture was filtered through Celite® and the filter cake was washed with DCM/MeOH (10/1, 50 mL). The filtrate was concentrated in vacuo to give the crude, which was purified by column chromatography (eluent: PE/EtOAc=3/1) to give rac-tert-butyl (6-methoxy-2-((1S*,2S*)-2-(4-methylpyrimidin-2-yl)cyclopropyl)quinolin-7-yl)carbamate (207 mg, 82%) as a yellow oil. ESI-MS [M+H]⁺: 407.2.

### Synthesis of rac-6-methoxy-2-((1S*,2S*)-2-(4-methylpyrimidin-2-yl)cyclopropyl)quinolin-7-amine

rac-tert-butyl (6-methoxy-2-((1S*,2S*)-2-(4-methylpyrimidin-2-yl)cyclopropyl)quinolin-7-yl)carbamate (207 mg, 0.51 mmol) in TFA (5 mL) was stirred at room temperature for 1 h. The mixture was concentrated in vacuo and the residue was with neutralized with NH₃ (7M in MeOH solution, 10 mL). The resulting mixture was concentrated in vacuo to give the crude, which was purified by column chromatography (eluent: DCM/MeOH=5/1) to give rac-6-methoxy-2-((1S*,2S*)-2-(4-methylpyrimidin-2-yl)cyclopropyl)quinolin-7-amine (135 mg, 86%) as a yellow solid. ESI-MS [M+H]⁺: 307.2.

### Synthesis of rac-1-(6-cyclopropyl-2-(((6-methoxy-2-((1S*,2S*)-2-(4-methylpyrimidin-2-yl)cycl propyl)quinolin-7-yl)amino)methyl)imidazo[1,2-a]pyridin-8-yl)-3-methylimidazolidine-2,4-dione

A mixture of rac-6-methoxy-2-((1S*,2S*)-2-(4-methylpyrimidin-2-yl)cyclopropyl)quinolin-7-amine (73 mg, 0.24 mmol) and 6-cyclopropyl-8-(3-methyl-2,4-dioxoimidazolidin-1-yl)imidazo[1,2-a]pyridine-2-carbaldehyde (72 mg, 0.24 mmol) in THF (10 mL) was added Ti(i-PrO)₄ (341 mg, 1.20 mmol) dropwise at room temperature. The mixture was stirred at 70° C. for 16 h. After cooling to room temperature, NaBH₃CN (45 mg, 0.72 mmol) and MeOH (2 mL) were added and the mixture was stirred at room temperature for 1 h. Water (50 mL) was added and the mixture was extracted with EtOAc (50 mL×3). The combined organic layers were washed with brine (40 mL), dried over anhydrous Na₂SO₄, and concentrated in vacuo to give the crude, which was purified by preparative TLC (eluent: PE/EtOAc=1/2) to give rac-1-(6-cyclopropyl-2-(((6-methoxy-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (70 mg, 50%) as a pink solid.

ESI-MS [M+H]⁺: 589.2. ¹H NMR (400 MHz, DMSO) δ 8.50 (d, J=5.1 Hz, 1H), 8.23 (d, J=1.3 Hz, 1H), 7.89 (d, J=8.2 Hz, 1H), 7.73 (s, 1H), 7.36 (d, J=1.5 Hz, 1H), 7.16-7.11 (m, 3H), 6.76 (s, 1H), 6.26 (t, J=5.8 Hz, 1H), 4.95 (s, 2H), 4.55 (d, J=5.7 Hz, 2H), 3.96 (s, 3H), 2.98 (s, 3H), 2.68-2.61 (m, 2H), 2.40 (s, 3H), 1.96-1.89 (m, 1H), 1.74-1.65 (m, 2H), 0.95-0.90 (m, 2H), 0.65-0.62 (m, 2H).

### Example 85

### Synthesis of rac-2-(7-(((6-cyclopropyl-8-(3-methyl-2,4-dioxoimidazolidin-1-yl)imidazo[1,2-a]pyridin-2-yl)methyl)amino)-2-((1S*,2S*)-2-(4-methylpyrimidin-2-yl)cyclopropyl)quinolin-4-yl)acetonitrile (I-85)

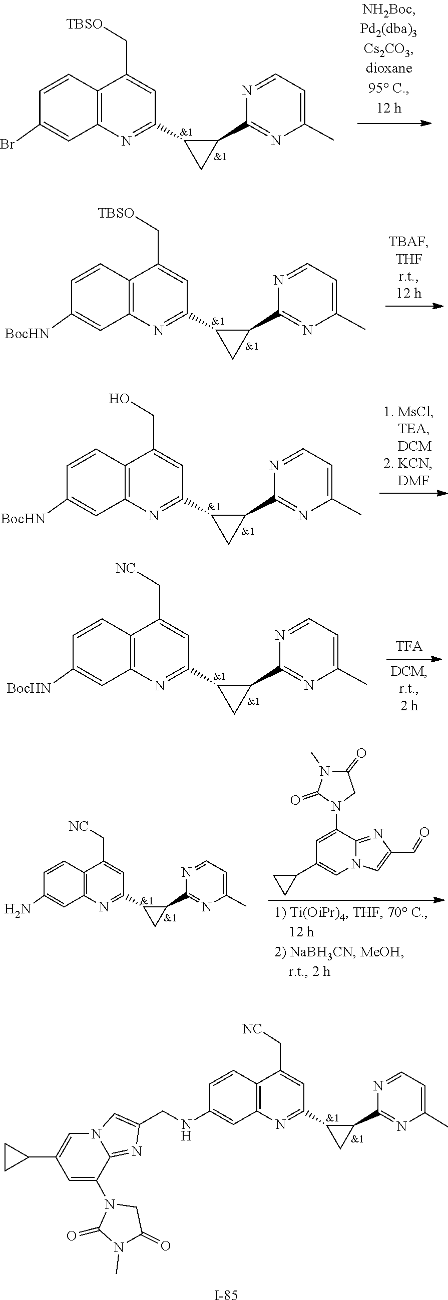

### Synthesis of rac-tert-butyl (4-(((tert-butyldimethylsilyl)oxy)methyl)-2-((1S*,2S*)-2-(4-methylpyrimidin-2-yl)cyclopropyl)quinolin-7-yl)carbamate

A mixture of 7-bromo-4-(((tert-butyldimethylsilyl)oxy)methyl)-2-(2-(4-methylpyrimidin-2-yl)cyclopropyl)quinoline (200 mg, 0.41 mmol), tert-butyl carbamate (73 mg, 0.62 mmol), Cs₂CO₃ (400 mg, 1.23 mmol), Pd(OAc)₂ (18 mg, 0.082 mmol) and Xphos (39 mg, 0.082 mmol) in dioxane (5 mL) was stirred at 95° C. for 2 h under N₂. After the reaction was cooled to room temperature, the mixture was filtered through Celite® and the filter cake was washed with DCM/MeOH (10/1, 50 mL). The filtrate was concentrated in vacuo to give the crude, which was purified by column chromatography ((eluent: EtOAc/PE=0 to 40%) to give rac-tert-butyl (4-(((tert-butyldimethylsilyl)oxy)methyl)-2-((1S*,2S*)-2-(4-methylpyrimidin-2-yl)cyclopropyl)quinolin-7-yl)carbamate (110 mg, 52%) as a yellow solid. ESI-MS [M+H]+: 521.3

### Synthesis of rac-tert-butyl (4-(hydroxymethyl)-2-((1S*,2S*)-2-(4-methylpyrimidin-2-yl)cyclopropyl)quinolin-7-yl)carbamate

To a solution of rac-tert-butyl (4-(((tert-butyldimethylsilyl)oxy)methyl)-2-((1S*,2S*)-2-(4-methylpyrimidin-2-yl)cyclopropyl)quinolin-7-yl)carbamate (200 mg, 0.38 mmol) in THF (5 mL) was added TBAF (0.7 mL, 0.7 mmol, 1N in THF) and the mixture was stirred at room temperature for 12 h. Water (30 mL) was added and the reaction was extracted with EtOAc (30 mL×3). The combined organic layers were washed with brine (30 mL), dried over Na₂SO₄ and concentrated in vacuo. The residue was purified by column chromatography (eluent: EtOAc/PE=0 to 50%) to give rac-tert-butyl (4-(hydroxymethyl)-2-((1S*,2S*)-2-(4-methylpyrimidin-2-yl)cyclopropyl)quinolin-7-yl)carbamate (150 mg, 97%) as a white solid. ESI-MS [M+H]+: 407.2.

### Synthesis of rac-tert-butyl (4-(cyanomethyl)-2-((1S*,2S*)-2-(4-methylpyrimidin-2-yl)cyclopropyl)quinolin-7-yl)carbamate

To a mixture of tert-butyl (4-(hydroxymethyl)-2-(2-(4-methylpyrimidin-2-yl)cyclopropyl)quinolin-7-yl)carbamate (150 mg, 0.37 mmol) and TEA (153 mg, 1.51 mmol) in DCM (5 mL) was added MsCl (90 mg, 0.79 mmol). The mixture was stirred at room temperature for 2 h and concentrated in vacuo. The residue was dissolved DMF (5 mL) and added KCN (48 mg, 0.74 mmol). The resulting mixture was stirred at room temperature for another 2 h and quenched with water (50 mL). The mixture was then extracted with EtOAc (30 mL×3). The combined organic layers were washed with brine (30 mL), dried over Na₂SO₄ and concentrated in vacuo to give rac-tert-butyl (4-(cyanomethyl)-2-((1S*,2S*)-2-(4-methylpyrimidin-2-yl)cyclopropyl)quinolin-7-yl)carbamate (210 mg, crude) as a yellow oil which was used in the next step directly. ESI-MS [M+H]+: 416.2

### Synthesis of rac-2-(7-amino-2-((1S*,2S*)-2-(4-methylpyrimidin-2-yl)cyclopropyl)quinolin-4-yl)acetonitrile

To a mixture of rac-tert-butyl (4-(cyanomethyl)-2-((1S*,2S*)-2-(4-methylpyrimidin-2-yl)cyclopropyl)quinolin-7-yl)carbamate (210 mg, crude) in DCM (5 mL) was added TFA (1 mL) and the mixture was stirred at room temperature for 1 h. The mixture was quenched with NaHCO₃ (sat. aq., 50 mL) and extracted with EtOAc (30 mL×3). The combined organic layers were washed with brine (30 mL), dried over Na₂SO₄ and concentrated in vacuo. The residue was purified by column chromatography (eluent: EtOAc/PE from 0 to 50%) to give rac-2-(7-amino-2-((1S*,2S*)-2-(4-methylpyrimidin-2-yl)cyclopropyl)quinolin-4-yl)acetonitrile (60 mg, 51% for two steps) as a yellow oil. ESI-MS [M+H]+: 316.1

### Synthesis of rac-2-(7-(((6-cyclopropyl-8-(3-methyl-2,4-dioxoimidazolidin-1-yl)imidazo[1,2-a]pyridin-2-yl)methyl)amino)-2-((1S*,2S*)-2-(4-methylpyrimidin-2-yl)cyclopropyl)quinolin-4-yl)acetonitrile

To a mixture of rac-2-(7-amino-2-((1S*,2S*)-2-(4-methylpyrimidin-2-yl)cyclopropyl)quinolin-4-yl)acetonitrile (20 mg, 0.063 mmol), 6-cyclopropyl-8-(3-methyl-2,4-dioxoimidazolidin-1-yl)imidazo[1,2-a]pyridine-2-carbaldehyde (27 mg, 0.09 mmol) in THF (5 mL) was added Ti(OiPr)₄ (85 mg, 0.3 mmol) and the mixture was stirred at 70° C. for 12 h under N₂. After the reaction was cooled to room temperature, NaBH₃CN (20 mg, 0.33 mmol) and MeOH (1 mL) were added and the mixture was stirred at room temperature for 2 h. The mixture was then filtered through Celite® and the filter cake was washed with DCM/MeOH (10/1, 30 mL). The filtrate was concentrated to give the crude, and purified by preparative TLC (eluent: EtOAc 100%) to give 2-(7-(((6-cyclopropyl-8-(3-methyl-2,4-dioxoimidazolidin-1-yl)imidazo[1,2-a]pyridin-2-yl)methyl)amino)-2-(2-(4-methylpyrimidin-2-yl)cyclopropyl)quinolin-4-yl)acetonitrile (12 mg, 32%) as a yellow solid.

ESI-MS [M+H]⁺: 598.3. ¹H NMR (400 MHz, DMSO) δ 8.52 (d, J=8.0 Hz, 1H), 8.33 (s, 1H), 8.25 (s, 1H), 7.76-7.68 (m, 2H), 7.35 (s, 1H), 7.19-7.13 (m, 3H), 6.94-6.91 (m, 1H), 6.85 (s, 1H), 4.94 (s, 2H), 4.51 (d, J=8 Hz 2H), 4.38 (s, 2H), 2.98 (s, 3H), 2.71-2.68 (m, 2H), 2.42 (s, 3H), 1.96-1.90 (m, 1H), 1.78-1.71 (m, 2H), 0.95-0.92 (m, 2H), 0.66-0.64 (m, 2H).

### Example 86

### Synthesis of rac-1-(6-cyclopropyl-2-(((5-hydroxy-7-((1S*,2S*)-2-(4-methylpyrimidin-2-yl)cyclopropyl)pyrido[2,3-d]pyrimidin-2-yl)amino)methyl)imidazo[1,2-a]pyridin-8-yl)-3-methylimidazolidine-2,4-dione (I-86)

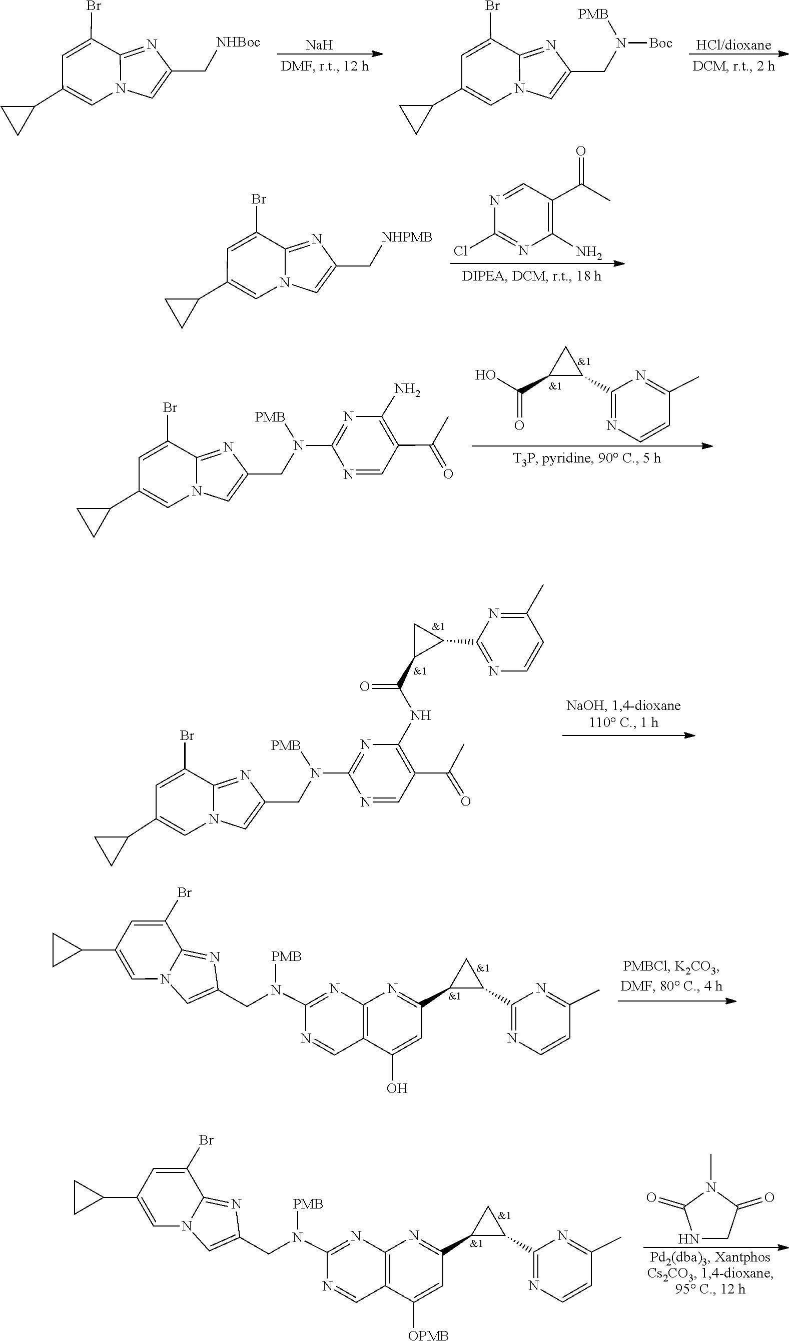

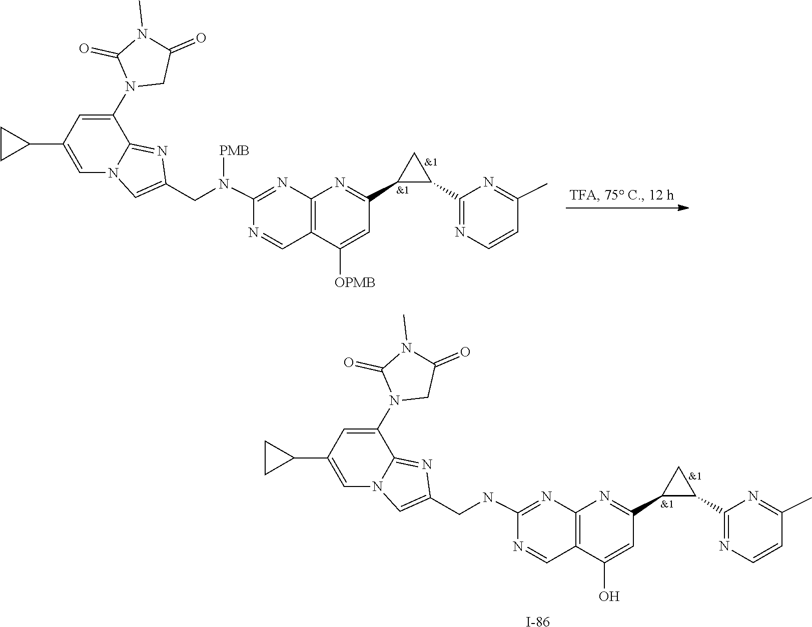

### Synthesis of tert-butyl ((8-bromo-6-cyclopropylimidazo[1,2-a]pyridin-2-yl)methyl)(4-methoxybenzyl)carbamate

To a mixture of tert-butyl ((8-bromo-6-cyclopropylimidazo[1,2-a]pyridin-2-yl)methyl)carbamate (2.0 g, 5.46 mmol) in DMF (50 mL) was added NaH (328 mg, 60% in mineral oil, 8.19 mmol) portionwise and the mixture was stirred at room temperature for 1 h. 1-(chloromethyl)-4-methoxybenzene (1.3 g, 8.19 mmol) was added and the resulting reaction mixture was stirred at room temperature for 12 h. The reaction was quenched with NH₄Cl (sat. aq., 100 mL) and extracted with EtOAc (100 mL×3). The combined organic layers were washed with brine (100 mL), dried over Na₂SO₄ then concentrated in vacuo to give the crude, which was purified by column chromatography (eluent: DCM/MeOH=0-3%) to give tert-butyl ((8-bromo-6-cyclopropylimidazo[1,2-a]pyridin-2-yl)methyl)(4-methoxybenzyl)carbamate (2.3 g, 88%) as a yellow solid. ESI-MS [M+H]+: 486.2

### Synthesis of tert-butyl ((8-bromo-6-cyclopropylimidazo[1,2-a]pyridin-2-yl)methyl)(4-methoxybenzyl)carbamate

A solution of tert-butyl ((8-bromo-6-cyclopropylimidazo[1,2-a]pyridin-2-yl)methyl)(4-methoxybenzyl)carbamate (2.3 g, 47 mmol) in HCl/1,4-dioxane (10 mL, 4 M in 1,4-dioxane) was stirred at room temperature for 2 h. The reaction mixture was concentrated in vacuo to give 1-(8-bromo-6-cyclopropylimidazo[1,2-a]pyridin-2-yl)-N-(4-methoxybenzyl)methanamine hydrochloride (2.3 g, crude) as a yellow solid. ESI-MS [M+H]+: 386.2

### Synthesis of 1-(4-amino-2-(((8-bromo-6-cyclopropylimidazo[1,2-a]pyridin-2-yl)methyl)(4-methoxybenzyl)amino)pyrimidin-5-yl)ethan-1-one

A mixture of 1-(4-amino-2-chloropyrimidin-5-yl)ethan-1-one (400 mg, 2.34 mmol), 1-(8-bromo-6-cyclopropylimidazo[1,2-a]pyridin-2-yl)-N-(4-methoxybenzyl)methanamine (900 mg, 2.34 mmol), DIPEA (906 mg, 7.02 mmol) in DCM (20 mL) was stirred at room temperature for 18 h. The reaction was washed with water (30 mL), extracted with DCM (40 mL×3). The combined organic layers were washed with brine (30 mL), dried over Na₂SO₄, filtered and concentrated in vacuo to give the crude, which was purified by preparative TLC (eluent: DCM/MeOH=30/1) to give 1-(4-amino-2-(((8-bromo-6-cyclopropylimidazo[1,2-a]pyridin-2-yl)methyl)(4-methoxybenzyl)amino)pyrimidin-5-yl)ethan-1-one (850 mg, 70%) as a white solid.

ESI-MS [M+H]+: 521.2.

### Synthesis of rac-(1S*,2S*)—N-(5-acetyl-2-(((8-bromo-6-cyclopropylimidazo[1,2-a]pyridin-2-yl)methyl)(4-methoxybenzyl)amino)pyrimidin-4-yl)-2-(4-methylpyrimidin-2-yl)cyclopropane-1-carboxamide

To a mixture of 1-(4-amino-2-(((8-bromo-6-cyclopropylimidazo[1,2-a]pyridin-2-yl)methyl)(4-methoxybenzyl)amino)pyrimidin-5-yl)ethan-1-one (800 mg, 1.53 mmol), rac-(1S*,2S*)-2-(4-methylpyrimidin-2-yl)cyclopropane-1-carboxylic acid (272 mg, 1.53 mmol) in pyridine (5 mL) was added T₃P (9.73 g, 15.3 mmol, 50% solution in EtOAc). The reaction solution was stirred at 90° C. for 5 h under N₂. The reaction was quenched with water (40 mL), extracted with EtOAc (40 mL×3). The combined organic layers were washed with brine (30 mL), dried over anhydrous Na₂SO₄, and concentrated in vacuo to give the crude product, which was purified by preparative TLC (eluent: DCM/MeOH=20/1) to give rac-(1S*,2S*)—N-(5-acetyl-2-(((8-bromo-6-cyclopropylimidazo[1,2-a]pyridin-2-yl)methyl)(4-methoxybenzyl)amino)pyrimidin-4-yl)-2-(4-methylpyrimidin-2-yl)cyclopropane-1-carboxamide (500 mg, 48%) as a yellow solid. ESI-MS [M+H]+: 681.2.

### Synthesis of rac-2-(((8-bromo-6-cyclopropylimidazo[1,2-a]pyridin-2-yl)methyl)(4-methoxy benzyl)amino)-7-((1S*,2S*)-2-(4-methylpyrimidin-2-yl)cyclopropyl)pyrido[2,3-d]pyrimidin-5-ol

To a solution of rac-(1S*,2S*)—N-(5-acetyl-2-(((8-bromo-6-cyclopropylimidazo[1,2-a]pyridin-2-yl)methyl)(4-methoxybenzyl)amino)pyrimidin-4-yl)-2-(4-methylpyrimidin-2-yl)cyclo propane-1-carboxamide (500 mg, 0.73 mmol) in 1,4-dioxane (10 mL) and was added NaOH (88 mg, 2.2 mmol). The reaction mixture was stirred at 110° C. for 1 h. The reaction was washed with NH₄Cl (sat. aq., 50 mL) extracted with EtOAc (50 mL×3). The combined organic layers were washed with brine (30 mL), dried over anhydrous Na₂SO₄, and concentrated in vacuo to give the crude product, which was purified by preparative TLC (eluent: DCM/MeOH=20/1) to give rac-2-(((8-bromo-6-cyclopropyl imidazo[1,2-a]pyridin-2-yl)methyl)(4-methoxybenzyl)amino)-7-((1S*,2S*)-2-(4-methyl pyrimidin-2-yl)cyclopropyl)pyrido[2,3-d]pyrimidin-5-ol (100 mg, 21%) as a yellow solid. ESI-MS [M+H]+: 663.2.

### Synthesis of rac-N-((8-bromo-6-cyclopropylimidazo[1,2-a]pyridin-2-yl)methyl)-N-(4-methoxybenzyl)-5-((4-methoxybenzyl)oxy)-7-((1S*,2S*)-2-(4-methylpyrimidin-2-yl)cyclopropyl)pyrido[2,3-d]pyrimidin-2-amine

To a solution of rac-2-(((8-bromo-6-cyclopropylimidazo[1,2-a]pyridin-2-yl)methyl)(4-methoxybenzyl)amino)-7-((1S*,2S*)-2-(4-methylpyrimidin-2-yl)cyclopropyl)pyrido[2,3-d]pyrimidin-5-ol (70 mg, 0.11 mmol) in DMF (5 mL) and was added K₂CO₃ (46 mg, 0.3 mmol) and PMBCl (34 mg, 0.22 mmol). The reaction mixture was stirred at 80° C. for 4 h. The reaction was washed with water (30 mL) and extracted with EtOAc (40 mL×3). The combined organic layers were washed with brine (30 mL), dried over anhydrous Na₂SO₄, and concentrated in vacuo to give the crude product, which was purified by preparative TLC (eluent: DCM/MeOH=30/1) to give rac-N-((8-bromo-6-cyclopropyl imidazo[1,2-a]pyridin-2-yl)methyl)-N-(4-methoxybenzyl)-5-((4-methoxybenzyl)oxy)-7-((1S*,2S*)-2-(4-methylpyrimidin-2-yl)cyclopropyl)pyrido[2,3-d]pyrimidin-2-amine (40 mg, 47%) as a yellow solid.

ESI-MS [M+H]+: 783.2.

### Synthesis of 1-(6-cyclopropyl-2-(((4-methoxybenzyl)(5-((4-methoxybenzyl)oxy)-7-(2-(4-methylpyrimidin-2-yl)cyclopropyl)pyrido[2,3-d]pyrimidin-2-yl)amino)methyl)imidazo[1,2-a]pyridin-8-yl)-3-methylimidazolidine-2,4-dione

To a mixture of rac-N-((8-bromo-6-cyclopropylimidazo[1,2-a]pyridin-2-yl)methyl)-N-(4-methoxybenzyl)-5-((4-methoxybenzyl)oxy)-7-((1S*,2S*)-2-(4-methylpyrimidin-2-yl)cyclopropyl)pyrido[2,3-d]pyrimidin-2-amine (40 mg, 0.05 mmol), 3-methylimidazolidine-2,4-dione (17 mg, 0.15 mmol) and Cs₂CO₃ (49 mg, 0.15 mmol) in 1,4-dioxane (5 mL) was added Pd₂(dba)₃ (6.4 mg, 0.007 mmol) and Xantphos (8.7 mg, 0.015 mmol). The reaction mixture was stirred at 95° C. for 12 h under N₂. The reaction mixture was filtered through Celite® and the filter cake was washed with DCM/MeOH (20/1, 40 mL). The filtrate was concentrated in vacuo to give the crude, which was purified by preparative TLC (eluent: DCM/MeOH=20/1) to give rac-1-(6-cyclopropyl-2-(((4-methoxybenzyl)(5-((4-methoxybenzyl)oxy)-7-((1S*,2S*)-2-(4-methylpyrimidin-2-yl)cyclopropyl) pyrido[2,3-d]pyrimidin-2-yl)amino)methyl)imidazo[1,2-a]pyridin-8-yl)-3-methylimidazolidine-2,4-dione (17 mg, 41%) as a yellow solid. ESI-MS [M+H]+: 817.2.

### Synthesis of rac-1-(6-cyclopropyl-2-(((5-hydroxy-7-((1S*,2S*)-2-(4-methylpyrimidin-2-yl)cyclopropyl)pyrido[2,3-d]pyrimidin-2-yl)amino)methyl)imidazo[1,2-a]pyridin-8-yl)-3-methylimidazolidine-2,4-dione

A solution of rac-1-(6-cyclopropyl-2-(((4-methoxybenzyl)(5-((4-methoxybenzyl)oxy)-7-((1S*,2S*)-2-(4-methylpyrimidin-2-yl)cyclopropyl)pyrido[2,3-d]pyrimidin-2-yl)amino)methyl) imidazo[1,2-a]pyridin-8-yl)-3-methylimidazolidine-2,4-dione (17 mg, 0.021 mmol) in TFA (2 mL) was stirred at 75° C. for 12 h under N₂. The reaction was concentrated in vacuo, and the residue was diluted with NaHCO₃ (sat. aq., 20 mL), extracted with DCM (20 mL×3). The combined organic layers was washed with brine (15 mL), dried over anhydrous Na₂SO₄, concentrated in vacuo to give the crude, which was purified by preparative TLC (eluent: DCM/MeOH=10/1) to give rac-1-(6-cyclopropyl-2-(((5-hydroxy-7-((1S*,2S*)-2-(4-methylpyrimidin-2-yl)cyclopropyl)pyrido[2,3-d]pyrimidin-2-yl)amino) methyl)imidazo[1,2-a]pyridin-8-yl)-3-methylimidazolidine-2,4-dione (5 mg, 42%) as a white solid.

ESI-MS [M+H]+: 577.2. ¹H NMR (400 MHz, DMSO) δ 11.74 (s, 1H), 8.85 (s, 1H), 8.53 (d, J=4.4 Hz, 1H), 8.44-8.26 (m, 1H), 8.22 (s, 1), 7.72 (s, 1), 7.32 (s, 1H), 7.20 (d, J=4.5 Hz, 1H), 5.74 (s, 1H), 4.87 (s, 2H), 4.64 (s, 2H), 2.97 (s, 3H), 2.53-2.50 (m, 2H), 2.41 (s, 3H), 1.97-1.92 (m, 1H), 1.70-1.68 (m, 2H), 0.97-0.84 (m, 2H), 0.67-0.64 (m, 2H).

### Example 87

### Synthesis of 6-((1-(6-cyclopropylimidazo[1,2-a]pyridin-2-yl)ethyl)amino)-3-((1S,2S)-2-(4-methylpyrimidin-2-yl)cyclopropyl)-4H-benzo[e][1,2,4]thiadiazine 1,1-dioxide (I-87)

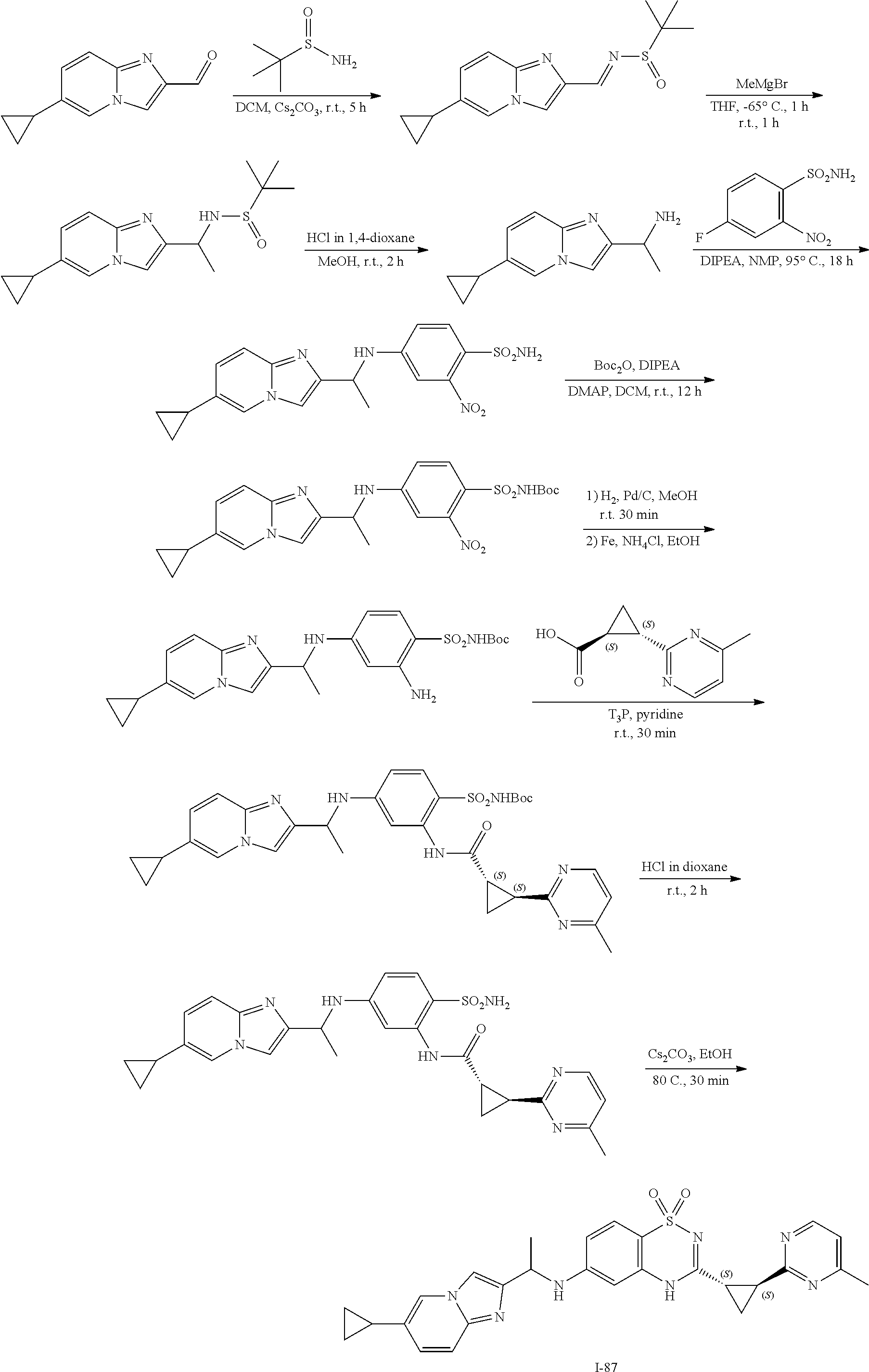

### Synthesis of (E)-N-((6-cyclopropylimidazo[1,2-a]pyridin-2-yl)methylene)-2-methylpropane-2-sulfinamide

To a mixture of 6-cyclopropylimidazo[1,2-a]pyridine-2-carbaldehyde (500 mg, 2.69 mmol) and 2-methylpropane-2-sulfinamide (423 mg, 3.49 mmol) in DCM (10 mL) was added Cs₂CO₃ (1.75 g, 5.38 mmol) and the mixture was stirred at room temperature for 5 h. Water (50 mL) was added and the reaction was extracted with extracted with EtOAc (30 mL×3). The combined organic layers were washed with brine (30 mL), dried over Na₂SO₄ and concentrated in vacuo. The residue was purified by column chromatography (eluent: EtOAc/PE=0 to 50%) to give (E)-N-((6-cyclopropylimidazo[1,2-a]pyridin-2-yl)methylene)-2-methylpropane-2-sulfinamide as yellow solid (760 mg, 98%). ESI-MS [M+H]⁺: 290.2

### Synthesis of N-(1-(6-cyclopropylimidazo[1,2-a]pyridin-2-yl)ethyl)-2-methylpropane-2-sulfinamide

To a mixture of (E)-N-((6-cyclopropylimidazo[1,2-a]pyridin-2-yl)methylene)-2-methylpropane-2-sulfinamide (750 mg, 2.58 mmol) in THF (15 mL) was added methylmagnesium bromide (2.5 mL, 3M in THF) at −65° C. The mixture was stirred at −65° C. for 1 h and then warmed to room temperature for another 1 h. The reaction mixture was quenched with NH₄Cl (sat. aq., 50 mL) and extracted with EtOAc (30 mL×3). The combined organic layers were washed with brine (30 mL), dried over Na₂SO₄ and concentrated in vacuo. The residue was purified by column chromatography (eluent: MeOH/DCM=0 to 5%) to give N-(1-(6-cyclopropylimidazo[1,2-a]pyridin-2-yl)ethyl)-2-methylpropane-2-sulfinamide as yellow solid (720 mg, yield 91%). ESI-MS [M+H]⁺: 306.2

### Synthesis of 1-(6-cyclopropylimidazo[1,2-a]pyridin-2-yl)ethan-1-amine

To a solution of N-(1-(6-cyclopropylimidazo[1,2-a]pyridin-2-yl)ethyl)-2-methylpropane-2-sulfinamide (720 mg, 2.36 mmol) in 1,4-dioxane (5 mL) was added HCl (5 mL, 20 mmoL, 4N in 1,4-dixane) and the mixture was stirred at room temperature for 1 h. The reaction mixture was concentrated in vacuo, the residue was basified with NH₃ (7 N in MeOH) and then concentrated to dryness. The residue was purified by column chromatography (eluent: MeOH/DCM=0 to 10%) to give 1-(6-cyclo propylimidazo[1,2-a]pyridin-2-yl)ethan-1-amine (400 mg, 85%) as a yellow solid. ESI-MS [M+H]⁺: 202.1

### Synthesis of 4-((1-(6-cyclopropylimidazo[1,2-a]pyridin-2-yl)ethyl)amino)-2-nitrobenzenesulfonamide

To a mixture of 1-(6-cyclopropylimidazo[1,2-a]pyridin-2-yl)ethan-1-amine (550 mg, 2.73 mmol) and 4-fluoro-2-nitrobenzenesulfonamide (501 mg, 2.28 mmol) in NMP (10 mL) was added DIPEA (588 mg, 4.56 mmol) and the mixture was stirred at 95° C. for 18 h. The reaction mixture was quenched with water (100 mL) and extracted with EtOAc (30 mL×3). The combined organic layers were washed with brine (30 mL), dried over Na₂SO₄ and concentrated in vacuo. The residue was purified by column chromatography (eluent: MeOH/DCM=0 to 10%) to give 4-((1-(6-cyclopropyl imidazo[1,2-a]pyridin-2-yl)ethyl)amino)-2-nitrobenzenesulfonamide as a yellow solid (450 mg, yield 49%). ESI-MS [M+H]⁺: 402.1

### Synthesis of tert-butyl ((4-((1-(6-cyclopropylimidazo[1,2-a]pyridin-2-yl)ethyl)amino)-2-nitrophenyl)sulfonyl)carbamate

To a solution of 4-((1-(6-cyclopropylimidazo[1,2-a]pyridin-2-yl)ethyl)amino)-2-nitrobenzenesulfonamide (150 mg, 0.37 mmol) in DCM (10 mL) was added TEA (113 mg, 1.12 mmol), DMAP (9 mg, 0.07 mmol), Boc₂O (163 mg, 0.75 mmol). Then the mixture was stirred at room temperature for 12 h. The reaction mixture was quenched with water (100 mL) and extracted with EtOAc (30 mL×3). The combined organic layers were washed with brine (30 mL), dried over Na₂SO₄ and concentrated in vacuo. The residue was purified by column chromatography (eluent: MeOH/DCM=0 to 5%) to give tert-butyl ((4-((1-(6-cyclopropylimidazo[1,2-a]pyridin-2-yl)ethyl)amino)-2-nitrophenyl)sulfonyl)carbamate as yellow solid (100 mg, 54%). ESI-MS [M+H]⁺: 502.2

### Synthesis of tert-butyl ((2-amino-4-((1-(6-cyclopropylimidazo[1,2-a]pyridin-2-yl)ethyl)amino)phenyl)sulfonyl)carbamate

A mixture of tert-butyl ((4-((1-(6-cyclopropylimidazo[1,2-a]pyridin-2-yl)ethyl)amino)-2-nitrophenyl)sulfonyl)carbamate (100 mg, 0.20 mmol) and Pd/C (50 mg, 50 wt %) in THF/MeOH (4 mL/4 mL) was stirred at room temperature for 1 h under H₂. Then the reaction mixture was filtered through Celite® and the filtrate was concentrated in vacuo. The residue was dissolved in EtOH (10 mL) and added Fe (56 mg, 1.00 mmol) and NH₄Cl (106 mg, 2.00 mmol). The resulting reaction mixture was stirred at 80° C. for 30 min under N₂. After cooled to room temperature, the reaction mixture was filtered through Celite® and the filter cake was washed with DCM/MeOH (10/1, 30 mL). The filtrate was concentrated in vacuo to give the crude, which was purified by preparative TLC (eluent: DCM/MeOH=10:1) to give tert-butyl ((2-amino-4-((1-(6-cyclopropylimidazo[1,2-a]pyridin-2-yl)ethyl)amino)phenyl)sulfonyl)carbamate as yellow solid (41 mg, yield 44%). ESI-MS [M+H]⁺: 472.2

### Synthesis of tert-butyl ((4-((1-(6-cyclopropylimidazo[1,2-a]pyridin-2-yl)ethyl)amino)-2-((1S,2S)-2-(4-methylpyrimidin-2-yl)cyclopropane-1-carboxamido)phenyl)sulfonyl)carbamate

To a mixture of tert-butyl ((2-amino-4-((1-(6-cyclopropylimidazo[1,2-a]pyridin-2-yl)ethyl)amino)phenyl)sulfonyl)carbamate (30 mg, 0.064 mmol) and (1S,2S)-2-(4-methylpyrimidin-2-yl)cyclopropane-1-carboxylic acid (11 mg, 0.06 mmol) in pyridine (0.3 mL) was added T₃P (607 mg, 0.95 mmol, 50% in EtOAc), the mixture was stirred at room temperature for 30 min. Then the reaction mixture was quenched with water (50 mL) and extracted with EtOAc (30 mL×3). The combined organic layers were washed with brine (30 mL), dried over Na₂SO₄ and concentrated in vacuo. The residue was purified by preparative TLC (eluent: DCM:MeOH=10:1) to give tert-butyl ((4-((1-(6-cyclopropylimidazo[1,2-a]pyridin-2-yl)ethyl)amino)-2-((1S,2S)-2-(4-methylpyrimidin-2-yl)cyclopropane-1-carboxamido)phenyl) sulfonyl)carbamate as yellow solid (22 mg, yield 58%). ESI-MS [M+H]⁺: 632.3

### Synthesis of (1S,2S)—N-(5-((1-(6-cyclopropylimidazo[1,2-a]pyridin-2-yl)ethyl)amino)-2-sulfamoylphenyl)-2-(4-methylpyrimidin-2-yl)cyclopropane-1-carboxamide

To a solution of ((4-((1-(6-cyclopropylimidazo[1,2-a]pyridin-2-yl)ethyl)amino)-2-((1S,2S)-2-(4-methylpyrimidin-2-yl)cyclopropane-1-carboxamido)phenyl)sulfonyl)carbamate (22 mg, 0.035 mmol) in MeOH (1 mL) was added HCl (1 mL, 4N in 1,4-dioxane) and the mixture was stirred at room temperature for 2 h. The reaction mixture was concentrated in vacuo to give (1S,2S)—N-(5-((1-(6-cyclopropylimidazo[1,2-a]pyridin-2-yl)ethyl)amino)-2-sulfamoylphenyl)-2-(4-methylpyrimidin-2-yl)cyclopropane-1-carboxamide (20 mg, crude) as a yellow solid, which was used directly in the next step.

ESI-MS [M+H]⁺: 532.2

### Synthesis of 6-((1-(6-cyclopropylimidazo[1,2-a]pyridin-2-yl)ethyl)amino)-3-((1S,2S)-2-(4-methylpyrimidin-2-yl)cyclopropyl)-4H-benzo[e][1,2,4]thiadiazine 1,1-dioxide

To a solution of (1S,2S)—N-(5-((1-(6-cyclopropylimidazo[1,2-a]pyridin-2-yl)ethyl)amino)-2-sulfamoylphenyl)-2-(4-methylpyrimidin-2-yl)cyclopropane-1-carboxamide (20 mg, crude) in EtOH (2 mL) was added Cs₂CO₃ (49 mg, 0.15 mmol) and the mixture was stirred at 80° C. for 30 min. The reaction mixture was filtered through Celite® and the filter cake was washed with DCM/MeOH (10/1, 30 mL). The filtrate was concentrated in vacuo to give the crude, which was purified by preparative TLC (eluent: DCM/MeOH=10:1) to give 6-((1-(6-cyclopropylimidazo[1,2-a]pyridin-2-yl)ethyl)amino)-3-((1S,2S)-2-(4-methylpyrimidin-2-yl)cyclopropyl)-4H-benzo[e][1,2,4]thiadiazine 1,1-dioxide (8 mg, yield 45% for 2 steps) as a white solid.

ESI-MS [M+H]⁺: 514.2. ¹H NMR (400 MHz, DMSO) δ 11.90 (s, 1H), 8.57-8.50 (m, 1H), 8.26 (d, J=10.5 Hz, 1H), 7.52 (d, J=6.3 Hz, 1H), 7.43-7.32 (m, 2H), 7.25-7.20 (m, 1H), 7.07 (d, J=6.8 Hz, 1H), 7.00-6.92 (m, 1H), 6.75-6.67 (m, 1H), 6.19 (s, 1H), 4.67-4.53 (m, 1H), 2.65-2.58 (m, 1H), 2.46-2.43 (m, 1H), 2.42 (d, J=4.5 Hz, 3H), 1.94-1.83 (m, 1H), 1.70-1.62 (m, 2H), 1.58-1.51 (m, 3H), 0.92-0.85 (m, 2H), 0.68-0.58 (m, 2H).

### Example 88

### Synthesis of rac-1-(6-cyclopropyl-2-(((5-(dimethylamino)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (I-88)

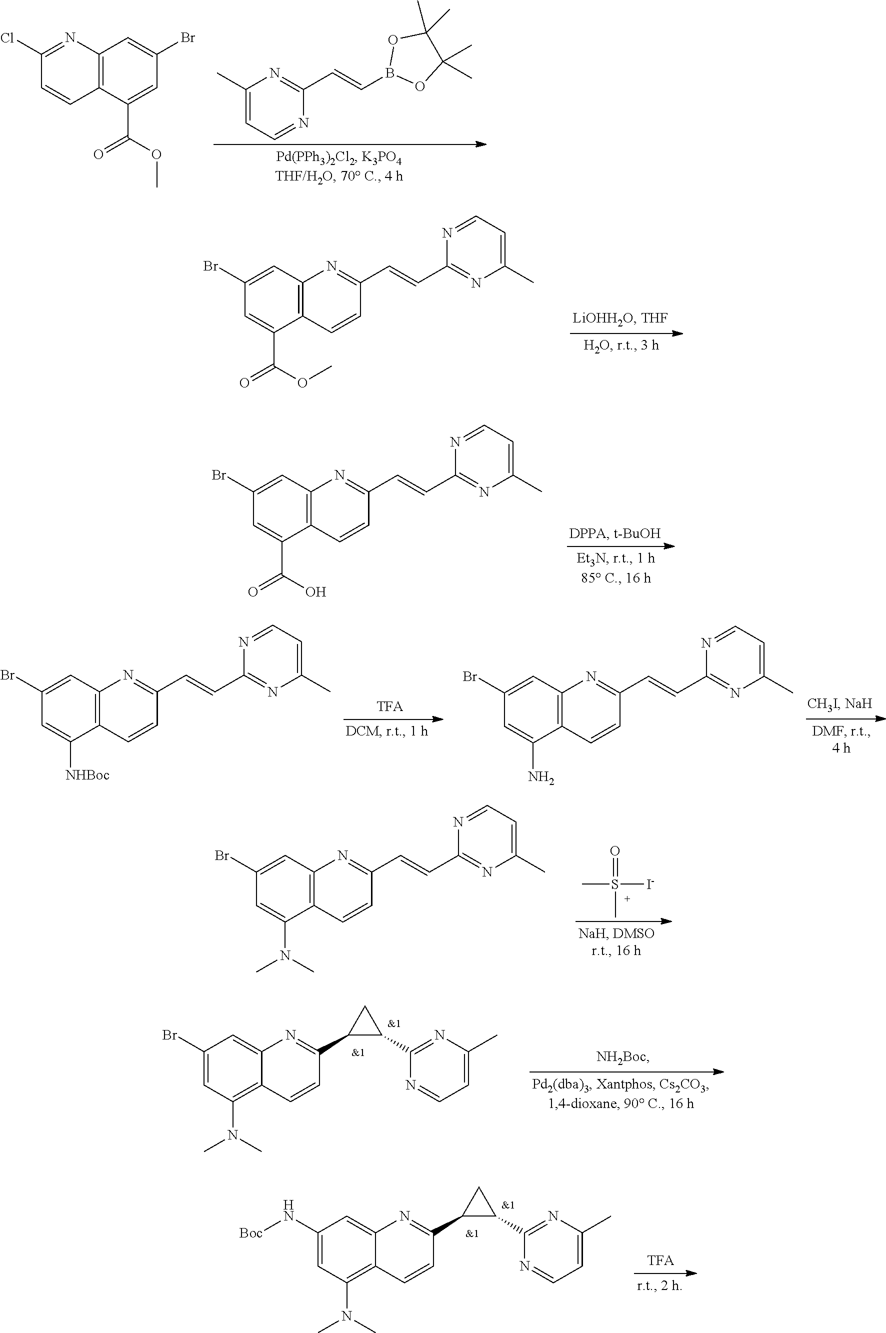

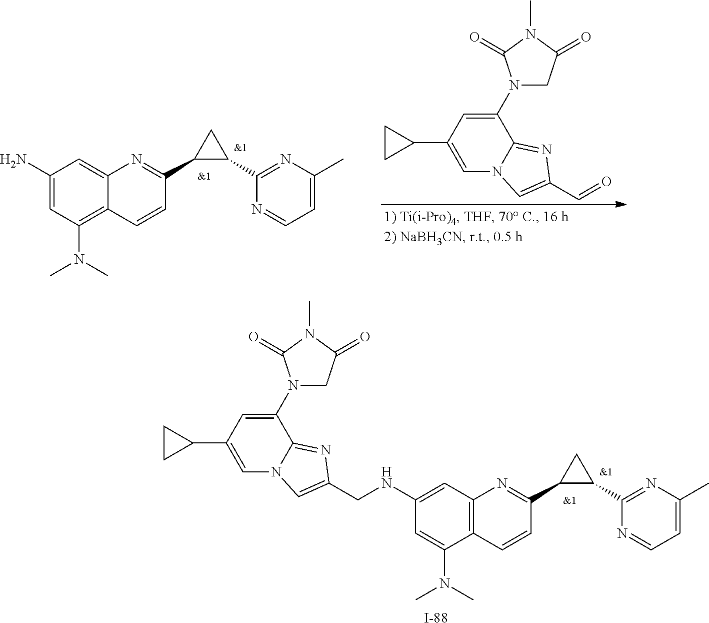

### Synthesis of methyl (E)-7-bromo-2-(2-(4-methylpyrimidin-2-yl)vinyl)quinoline-5-carboxylate

To a mixture of methyl 7-bromo-2-chloroquinoline-5-carboxylate (3.1 g, 10.3 mmol) in THF/H₂O (150 mL/15 mL) was added (E)-4-methyl-2-(2-(4,4,5,5-tetramethyl-1,3,2-dioxaborolan-2-yl)vinyl)pyrimidine (3.05 g, 12.4 mmol), Pd(PPh₃)₂Cl₂ (732 mg, 1.0 mmol) and K₃PO₄ (6.6 g, 30.9 mmol). The reaction mixture was stirred at 70° C. for 4 h under N₂. The mixture was cooled to room temperature, filtered through Celite® and the filter cake was washed with DCM/MeOH (10/1, 100 mL). The filtrate was concentrated in vacuo to give the crude, which was purified by column chromatography (eluent: PE/EtOAc=20/1˜5/1) to give methyl (E)-7-bromo-2-(2-(4-methylpyrimidin-2-yl)vinyl)quinoline-5-carboxylate (2.22 g, 56%) as a yellow solid. ESI-MS [M+H]+: 384.0

### Synthesis of (E)-7-bromo-2-(2-(4-methylpyrimidin-2-yl)vinyl)quinoline-5-carboxylic acid

To a mixture of methyl (E)-7-bromo-2-(2-(4-methylpyrimidin-2-yl)vinyl)quinoline-5-carboxylate (2.22 g, 5.78 mmol) in THF/H₂O (20 mL/20 mL) was added LiOH—H₂O (486 mg, 11.56 mmol). The resulting mixture was stirred at room temperature for 3 h. The mixture was adjusted pH=3 with 1N HCl solution then concentrated in vacuo to give (E)-7-bromo-2-(2-(4-methylpyrimidin-2-yl)vinyl)quinoline-5-carboxylic acid (2.6 g, crude) as a yellow solid. ESI-MS [M+H]+: 370.0

### Synthesis of tert-butyl (E)-(7-bromo-2-(2-(4-methylpyrimidin-2-yl)vinyl)quinolin-5-yl)carbamate

To a solution of (E)-7-bromo-2-(2-(4-methylpyrimidin-2-yl)vinyl)quinoline-5-carboxylic acid (2.6 g, crude) in t-BuOH (60 mL) was added DPPA (3.18 g, 11.6 mmol) and Et₃N (2.4 mL, 17.34 mmol). The resulting mixture was stirred at room temperature for 1 h and then stirred at 85° C. for 16 h. After cooling to room temperature, the reaction was quenched with water (50 mL) and extracted with EtOAc (50 mL×3). The combined organic layers were washed with brine (50 mL), dried over anhydrous Na₂SO₄, and concentrated in vacuo to give the crude product, which was purified by column chromatography (eluent: DCM/MeOH=20/1) to give tert-butyl (E)-(7-bromo-2-(2-(4-methylpyrimidin-2-yl)vinyl)quinolin-5-yl)carbamate (600 mg, yield 24% over 2 steps) as a brown oil. ESI-MS [M+H]+: 441.0

### Synthesis of (E)-7-bromo-2-(2-(4-methylpyrimidin-2-yl)vinyl)quinolin-5-amine

A solution of tert-butyl (E)-(7-bromo-2-(2-(4-methylpyrimidin-2-yl)vinyl)quinolin-5-yl)carbamate (600 mg, 1.36 mmol) in DCM (10 mL) and TFA (1 mL) was stirred at room temperature for 1 h. The reaction was quenched with NaHCO₃ (sat. aq., 50 mL) and extracted with DCM (50 mL×3). The combined organic layers were washed with brine (50 mL), dried over anhydrous Na₂SO₄, and concentrated in vacuo to give the crude product, which was purified by column chromatography (eluent: DCM/MeOH=20/1) to give (E)-7-bromo-2-(2-(4-methylpyrimidin-2-yl)vinyl)quinolin-5-amine (390 mg, yield 84%) as a brown oil. ESI-MS [M+H]+: 341.0

### Synthesis of (E)-7-bromo-N,N-dimethyl-2-(2-(4-methylpyrimidin-2-yl)vinyl)quinolin-5-amine

To a mixture of (E)-7-bromo-2-(2-(4-methylpyrimidin-2-yl)vinyl)quinolin-5-amine (390 mg, 1.15 mmol) in DMF (10 mL) was added NaH (230 mg, 60% in mineral oil, 5.75 mmol) at 0° C. and the mixture was stirred at room temperature for 1 h. A solution of CH₃I (490 mg, 3.45 mmol) in DMF (5 mL) was added and the mixture was stirred at room temperature for 3 h. The reaction mixture was quenched with H₂O (50 mL) and extracted with EtOAc (30 mL×3). The combined organics were washed with brine (50 mL), dried over Na₂SO₄ and concentrated in vacuo. The residue was purified by column chromatography (eluent: PE,EtOAc=3/1) to give (E)-7-bromo-N,N-dimethyl-2-(2-(4-methylpyrimidin-2-yl)vinyl)quinolin-5-amine (300 mg, 71%) as a yellow oil. ESI-MS [M+H]+: 369.0

### Synthesis of rac-7-bromo-N,N-dimethyl-2-((1S*,2S*)-2-(4-methylpyrimidin-2-yl)cyclopropyl)quinolin-5-amine

To a suspension of trimethylsulfoxonium iodide (535 mg, 2.43 mmol) in DMSO (10 mL) was added NaH (97 mg, 60% in mineral oil, 2.43 mmol) at 0° C. under N₂ and the mixture was stirred at room temperature for 1 h. Then a solution of (E)-7-bromo-N,N-dimethyl-2-(2-(4-methylpyrimidin-2-yl)vinyl)quinolin-5-amine (300 mg, 0.81 mmol) in DMSO (5 mL) was added. The mixture was stirred at room temperature for 16 h then quenched with H₂O (50 mL) and extracted with EtOAc (50 mL×3). The combined organics were washed with brine (50 mL), dried over Na₂SO₄ and concentrated in vacuo to get the crude, which was purified by column chromatography (eluent: PE/EtOAc=5/1) to give rac-7-bromo-N,N-dimethyl-2-((1S*,2S*)-2-(4-methylpyrimidin-2-yl)cyclopropyl)quinolin-5-amine (100 mg, 32%) as a yellow oil. ESI-MS [M+H]+: 383.0

### Synthesis of rac-tert-butyl (5-(dimethylamino)-2-((1S*,2S*)-2-(4-methylpyrimidin-2-yl)cyclopropyl)quinolin-7-yl)carbamate

To a mixture of rac-7-bromo-N,N-dimethyl-2-((1S*,2S*)-2-(4-methylpyrimidin-2-yl)cyclopropyl)quinolin-5-amine (80 mg, 0.21 mmol), NH₂Boc (49 mg, 0.42 mmol) and Cs₂CO₃ (205 mg, 0.63 mmol) in 1,4-dioxane (10 mL) was added Pd₂(dba)₃ (38 mg, 0.042 mmol) and xantphos (49 mg, 0.084 mmol). The reaction mixture was stirred at 90° C. for 16 h under N₂. After cooling to room temperature, the reaction mixture was filtered through Celite® and the filter cake was washed with DCM/MeOH (10/1, 20 mL). The filtrate was concentrated in vacuo to give the crude, which was purified by column chromatography (eluent: PE/EtOAc=30/1˜3/1) to give rac-tert-butyl (5-(dimethylamino)-2-((1S*,2S*)-2-(4-methylpyrimidin-2-yl)cyclopropyl)quinolin-7-yl)carbamate (80 mg, 91%) as a yellow solid. ESI-MS [M+H]+: 420.2

### Synthesis of rac-N5,N5-dimethyl-2-((1S*,2S*)-2-(4-methylpyrimidin-2-yl)cyclopropyl)quinoline-5,7-diamine

A mixture of rac-tert-butyl (5-(dimethylamino)-2-((1S*,2S*)-2-(4-methylpyrimidin-2-yl)cyclopropyl)quinolin-7-yl)carbamate (80 mg, 0.19 mmol) in TFA (2 mL) was stirred at room temperature for 2 h. The reaction mixture was concentrated in vacuo, the residue was neutralized with NH₃ (7 M in MeOH, 10 mL) and concentrated in vacuo to give the crude, which was purified by preparative TLC (eluent: DCM/MeOH=10/1) to give rac-N5,N5-dimethyl-2-((1S*,2S*)-2-(4-methylpyrimidin-2-yl)cyclopropyl)quinoline-5,7-diamine (37 mg, 61%) as a yellow oil. ESI-MS [M+H]+: 320.1

### Synthesis of rac-1-(6-cyclopropyl-2-(((5-(dimethylamino)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione

To a mixture of rac-N⁵,N⁵-dimethyl-2-((1S*,2S*)-2-(4-methylpyrimidin-2-yl)cyclopropyl)quinoline-5,7-diamine (18 mg, 0.056 mmol) and 6-cyclopropyl-8-(3-methyl-2,4-dioxoimidazolidin-1-yl)imidazo[1,2-a]pyridine-2-carbaldehyde (17 mg, 0.056 mmol) in THF (5 mL) was added Ti(i-PrO)₄ (80 mg, 0.28 mmol). The resulting mixture was stirred at 70° C. for 16 h under N₂. The mixture was cooled to room temperature, NaBH₃CN (13 mg, 0.20 mmol) and MeOH (1 mL) were added, the resulting mixture was stirred at room temperature for 0.5 h. Water (20 mL) was added and extracted with EtOAc (30 mL×3). The combined organic layers were washed with brine (30 mL), dried over anhydrous Na₂SO₄, and concentrated in vacuo to give the crude, which was purified by preparative TLC (eluent: DCM/MeOH=20/1) to give rac-1-(6-cyclopropyl-2-(((5-(dimethylamino)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (11 mg, 32%) as a yellow solid.

ESI-MS [M+H]⁺: 602.3. ¹H NMR (400 MHz, DMSO) δ 8.47 (d, J=5.1 Hz, 1H), 8.23 (d, J=1.1 Hz, 1H), 8.01 (d, J=8.4 Hz, 1H), 7.73 (s, 1H), 7.31 (d, J=1.5 Hz, 1H), 7.13 (d, J=5.1 Hz, 1H), 7.03 (d, J=8.5 Hz, 1H), 6.65 (t, J=5.7 Hz, 1H), 6.61 (d, J=2.0 Hz, 1H), 6.41 (d, J=1.6 Hz, 1H), 4.92 (s, 2H), 4.43 (d, J=5.6 Hz, 2H), 2.94 (s, 3H), 2.72 (s, 6H), 2.66-2.59 (m, 2H), 2.37 (s, 3H), 1.93-1.86 (m, 1H), 1.72-1.64 (m, 2H), 0.92-0.87 (m, 2H), 0.63-0.59 (m, 2H).

### Example 89

### Synthesis of rac-7-chloro-6-(((6-cyclopropylimidazo[1,2-a]pyridin-2-yl)methyl)amino)-3-((1S*,2S*)-2-(4-methylpyrimidin-2-yl)cyclopropyl)-4H-benzo[e][1,2,4]thiadiazine 1,1-dioxide (I-89)

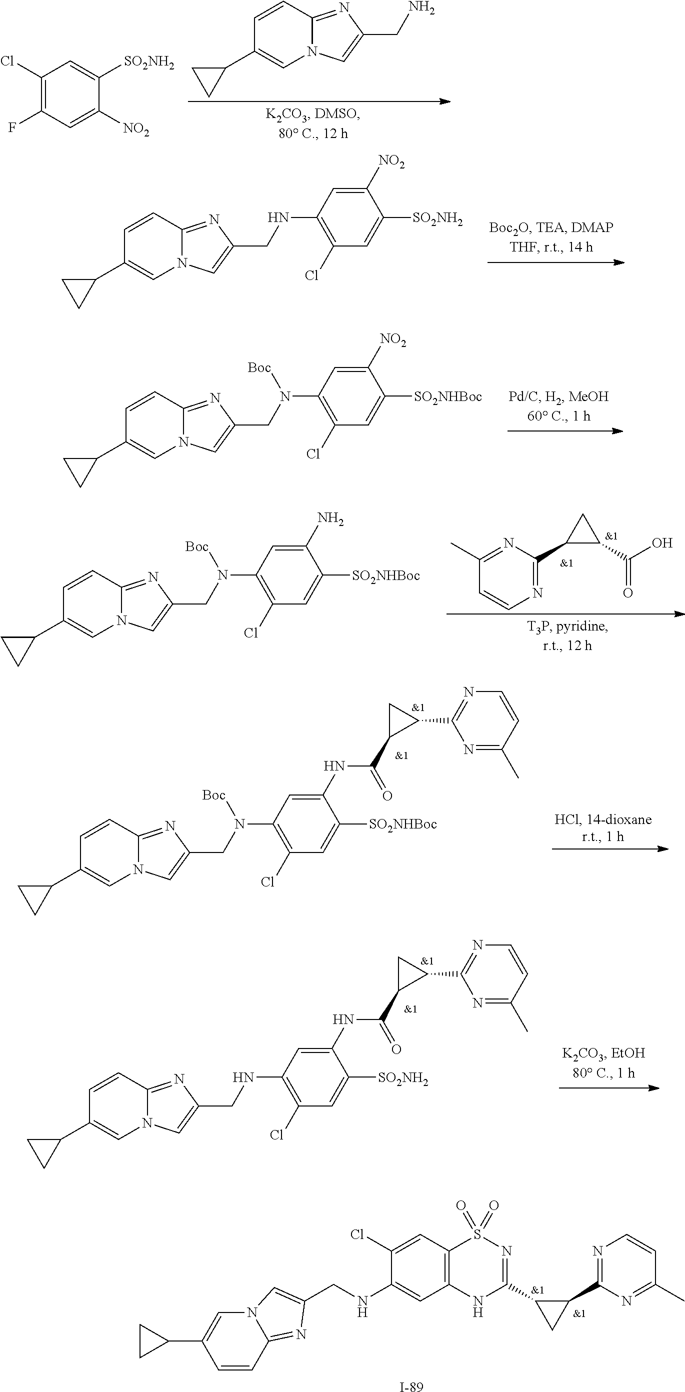

### Synthesis of 5-chloro-4-(((6-cyclopropylimidazo[1,2-a]pyridin-2-yl)methyl)amino)-2-nitrobenzenesulfonamide

To a mixture of 5-chloro-4-fluoro-2-nitrobenzenesulfonamide (600 mg, 2.36 mmol), (6-cyclopropylimidazo[1,2-a]pyridin-2-yl)methanamine (440 mg, 2.36 mmol) in DMSO (10 mL) was added K₂CO₃ (977 mg, 7.08 mmol). The reaction mixture was stirred at 80° C. for 12 h. The reaction was quenched with H₂O (40 mL), extracted with EtOAc (40 mL×3). The combined organic layers were washed with brine (40 mL), dried over anhydrous Na₂SO₄, and concentrated in vacuo to give the crude, which purified by column chromatography (eluent: DCM/MeOH=25/1) to give 5-chloro-4-(((6-cyclopropylimidazo[1,2-a]pyridin-2-yl)methyl)amino)-2-nitrobenzenesulfonamide (550 mg, 55%) as a yellow solid. ESI-MS [M+H]+: 422.2.

### Synthesis of tert-butyl (4-(N-(tert-butoxycarbonyl)sulfamoyl)-2-chloro-5-nitrophenyl)((6-cyclopropylimidazo[1,2-a]pyridin-2-yl)methyl)carbamate

To a mixture of 5-chloro-4-(((6-cyclopropylimidazo[1,2-a]pyridin-2-yl)methyl)amino)-2-nitrobenzenesulfonamide (550 mg, 1.3 mmol) in DCM (25 mL) was added di-tert-butyl dicarbonate (850 mg, 3.9 mmol), DMAP (16 mg, 0.13 mmol) and TEA (394 mg, 3.9 mmol. The reaction solution was stirred at room temperature for 14 h. The reaction was diluted with DCM (30 mL) then washed with H₂O (30 mL×2) then brine (30 mL), dried over anhydrous Na₂SO₄ and concentrated in vacuo to give the crude product, which purified by silica gel column chromatography (eluent: DCM/MeOH=50:1) to give tert-butyl (4-(N-(tert-butoxycarbonyl)sulfamoyl)-2-chloro-5-nitrophenyl)((6-cyclopropyl imidazo[1,2-a]pyridin-2-yl)methyl)carbamate (330 mg, 41%) as a yellow solid. ESI-MS [M+H]+: 622.2.

### Synthesis of tert-butyl (3-amino-4-(methylsulfonyl)phenyl)((6-cyclopropylimidazo[1,2-a]pyridin-2-yl)methyl)carbamate

To a mixture of tert-butyl (4-(N-(tert-butoxycarbonyl)sulfamoyl)-2-chloro-5-nitrophenyl)((6-cyclopropylimidazo[1,2-a]pyridin-2-yl)methyl)carbamate (300 mg, 0.48 mmol) in MeOH (20 mL) and was added Pd/C (50 mg). The reaction mixture was stirred at 60° C. for 1 h under H₂ atmosphere. The reaction mixture was filtered through Celite® and washed with MeOH (50 mL). The filtrate was concentrated in vacuo to give the crude, which purified by preparative TLC (DCM/MeOH=30/1) to give tert-butyl (5-amino-4-(N-(tert-butoxycarbonyl)sulfamoyl)-2-chlorophenyl)((6-cyclopropyl imidazo[1,2-a]pyridin-2-yl)methyl)carbamate (120 mg, 42%) as a yellow solid. ESI-MS [M+H]+: 592.2.

### Synthesis of rac-tert-butyl (4-(N-(tert-butoxycarbonyl)sulfamoyl)-2-chloro-5-((1S*,2S*)-2-(4-methyl pyrimidin-2-yl)cyclopropane-1-carboxamido)phenyl)((6-cyclopropylimidazo[1,2-a]pyridin-2-yl)methyl)carbamate

To a mixture of tert-butyl (5-amino-4-(N-(tert-butoxycarbonyl)sulfamoyl)-2-chlorophenyl) ((6-cyclopropylimidazo[1,2-a]pyridin-2-yl)methyl)carbamate (80 mg, 0.14 mmol), 2-(4-methyl pyrimidin-2-yl)cyclopropane-1-carboxylic acid (25 mg, 0.14 mmol) in pyridine (3 mL) was added T₃P (890 mg, 1.4 mmol, 50% solution in EtOAc). The reaction mixture was stirred at room temperature for 12 h under N₂. The reaction was quenched with water (20 mL) then extracted with EtOAc (30 mL×3). The combined organic layers were washed with brine (20 mL), dried over anhydrous Na₂SO₄, and concentrated to give the crude product, which was purified by preparative TLC (eluent: DCM/MeOH=15/1) to give rac-tert-butyl (4-(N-(tert-butoxycarbonyl)sulfamoyl)-2-chloro-5-((1S*,2S*)-2-(4-methylpyrimidin-2-yl)cyclopropane-1-carboxamido)phenyl)((6-cyclopropylimidazo [1,2-a]pyridin-2-yl)methyl)carbamate (45 mg, 43%) as a yellow solid. ESI-MS [M+H]+: 752.2.

### Synthesis of rac-(1S*,2S*)—N-(4-chloro-5-(((6-cyclopropylimidazo[1,2-a]pyridin-2-yl)methyl)amino)-2-sulfamoylphenyl)-2-(4-methylpyrimidin-2-yl)cyclopropane-1-carboxamide

To a solution of rac-tert-butyl (4-(N-(tert-butoxycarbonyl)sulfamoyl)-2-chloro-5-((1S*,2S*)-2-(4-methylpyrimidin-2-yl)cyclopropane-1-carboxamido)phenyl)((6-cyclopropyl imidazo[1,2-a]pyridin-2-yl)methyl)carbamate (45 mg, 0.06 mmol) in 1,4-dioxane (2 mL) was added HCl (0.5 mL, 4 M solution in 1,4-dioxane). The reaction mixture was stirred at room temperature for 1 h. The reaction was concentrated in vacuo. The residue was diluted with NaHCO₃ (sat. aq., 20 mL), extracted with DCM/MeOH (15/1, 30 mL×3). The combined organic layers were concentrated in vacuo to give the crude, which was purified with preparative TLC (eluent: DCM/MeOH=10/1) to give rac-(1S*,2S*)—N-(4-chloro-5-(((6-cyclopropylimidazo[1,2-a]pyridin-2-yl)methyl)amino)-2-sulfamoylphenyl)-2-(4-methylpyrimidin-2-yl)cyclopropane-1-carboxamide (30 mg, 91%) as a yellow solid. ESI-MS [M+H]⁺: 552.2

### Synthesis of rac-7-chloro-6-(((6-cyclopropylimidazo[1,2-a]pyridin-2-yl)methyl)amino)-3-((1S*,2S*)-2-(4-methylpyrimidin-2-yl)cyclopropyl)-4H-benzo[e][1,2,4]thiadiazine 1,1-dioxide

To a mixture of rac-(1S*,2S*)—N-(4-chloro-5-(((6-cyclopropylimidazo[1,2-a]pyridin-2-yl)methyl)amino)-2-sulfamoylphenyl)-2-(4-methylpyrimidin-2-yl)cyclopropane-1-carboxamide (30 mg, 0.054 mmol) in EtOH (3 mL) was added K₂CO₃ (21 mg, 0.15 mmol). The reaction mixture was stirred at 80° C. for 1 h. The reaction was cooled to room temperature and quenched with water (15 mL), extracted with EtOAc (20 mL×3). The combined organic layers were washed with brine (30 mL), dried over anhydrous Na₂SO₄, and concentrated in vacuo to give crude, which was purified with preparative TLC (eluent: DCM/MeOH=10/1) to give rac-7-chloro-6-(((6-cyclopropylimidazo[1,2-a]pyridin-2-yl)methyl)amino)-3-((1S*,2S*)-2-(4-methylpyrimidin-2-yl)cyclopropyl)-4H-benzo[e][1,2,4]thiadiazine 1,1-dioxide (10 mg, 34%) as a white solid.

ESI-MS [M+H]+: 534.2. ¹H NMR (400 MHz, DMSO) δ 8.53 (d, J=5.1 Hz, 1H), 8.26 (s, 1H), 7.61 (s, 1H), 7.56 (s, 1H), 7.37 (d, J=9.3 Hz, 1H), 7.21 (d, J=5.1 Hz, 1H), 7.03-6.87 (m, 2H), 6.37 (s, 1H), 4.50 (d, J=5.7 Hz, 2H), 2.66-2.58 (m, 2H), 2.42 (s, 3H), 1.61-1.71 (m, 1H), 1.66-1.64 (m, 2H), 0.90-0.84 (m, 2H). 0.67-0.60 (m, 2H).

### Example 90

### Synthesis of rac-1-(6-cyclopropyl-2-(((2-((1S*,2S*)-2-(4-methylpyrimidin-2-yl)cyclopropyl)quinolin-7-yl)amino)methyl)imidazo[1,2-a]pyridin-8-yl)-5-(3-hydroxyoxetan-3-yl)-3-methylimidazolidine-2,4-dione (I-90)

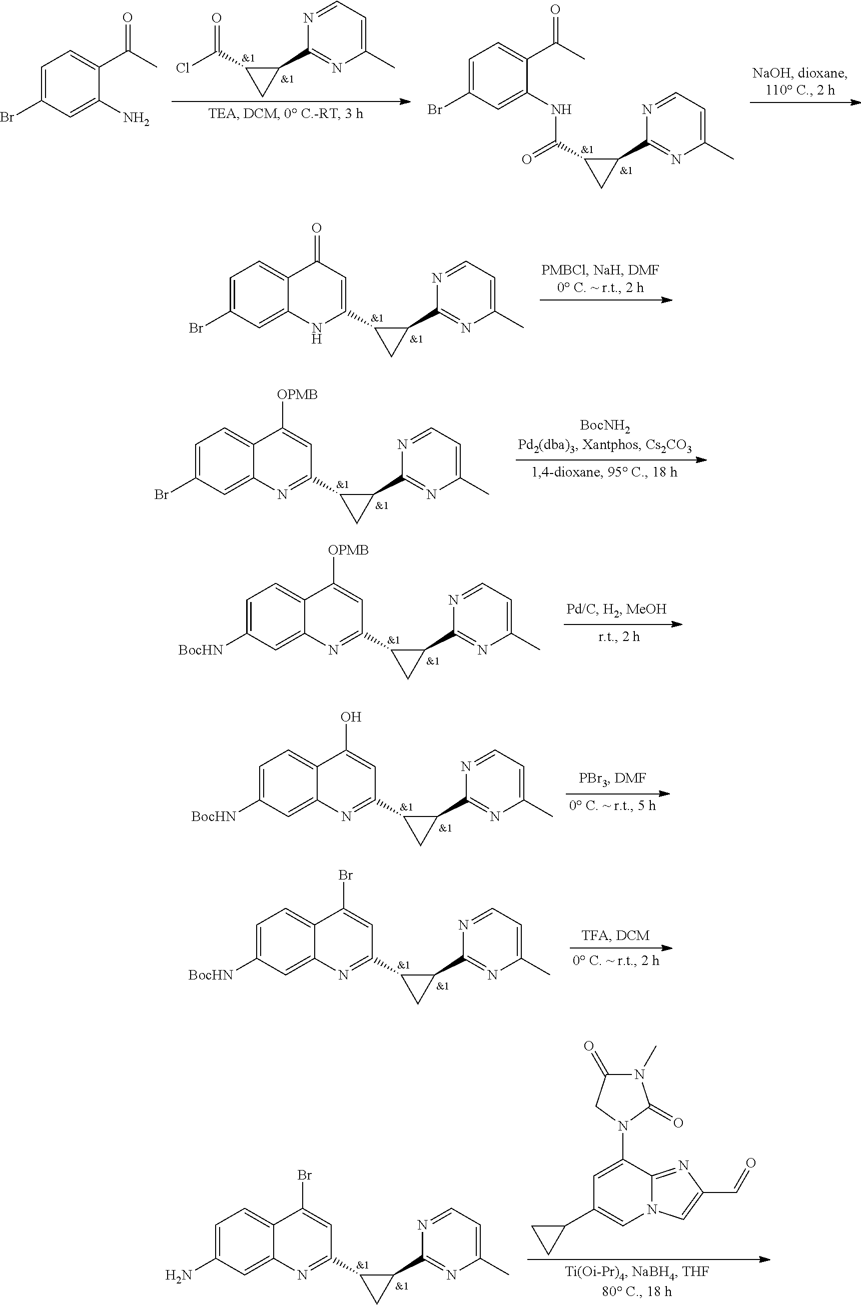

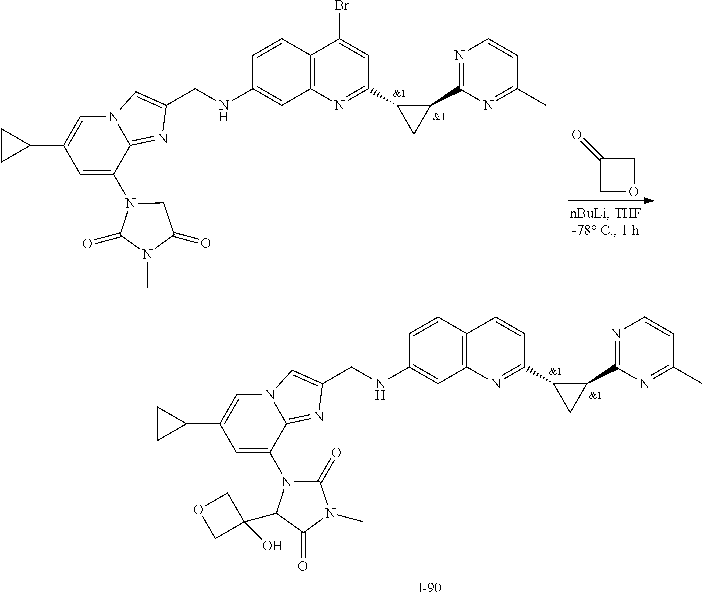

### Synthesis of rac-(1S*,2S*)—N-(2-acetyl-5-bromophenyl)-2-(4-methylpyrimidin-2-yl)cyclopropane-1-carboxamide

To a mixture of rac-(1S*,2S)-2-(4-methylpyrimidin-2-yl)cyclopropane-1-carboxylic acid (2.8 g, 16 mmol) in DCM (30 mL) was added oxalyl chloride (2 mL, 24 mmol) at 0° C., followed by DMF (0.5 mL). The mixture was stirred at room temperature for 3 h. The reaction was concentrated in vacuo to give the corresponding the acid chloride. The residue was re-dissolved in DCM (20 mL), which was added to a solution of 1-(2-amino-4-bromophenyl)ethan-1-one (2.5 g, 12 mmol) and TEA (3.6 g, 35 mmol) in DCM (35 mL). The mixture was stirred at room temperature for another 3 h. The reaction was quenched with NaHCO₃ (sat. aq., 100 mL), and then extracted DCM (50 mL×3). The combined organic layers were washed with brine (50 mL), dried over Na₂SO₄, filtered and concentrated in vacuo. The residue was purified by column chromatography on silica gel (eluent. EtOAc/PE=0-50%) to afford rac-(1S*,2S*)—N-(2-acetyl-5-bromophenyl)-2-(4-methylpyrimidin-2-yl)cyclopropane-1-carboxamide (2.4 g, yield 54%) as a pale-yellow solid. ESI-MS [M+H]+: 374.1

### Synthesis of rac-7-bromo-2-((1S*,2S*)-2-(4-methylpyrimidin-2-yl)cyclopropyl)quinolin-4 (1H)-one

To a mixture of rac-(1S*,2S*)—N-(2-acetyl-5-bromophenyl)-2-(4-methylpyrimidin-2-yl)cyclopropane-1-carboxamide (2.4 g, 6.4 mmol) in 1,4-dioxane (75 mL) was added NaOH (1 g, 25.6 mmol). The reaction mixture was stirred at 110° C. for 1 h. After cooling to room temperature, the reaction was diluted with H₂O (40 mL), and then neutralized with HCl (1 N aq., 20 mL) to pH=7. A yellow solid was precipitate formed which was isolated by filtration and washed with H₂O (20 mL). The filtrant was collected and dried in vacuo to afford rac-7-bromo-2-((1S*,2S*)-2-(4-methylpyrimidin-2-yl)cyclopropyl)quinolin-4 (1H)-one (2 g, yield 87%) as pale-yellow solid. ESI-MS [M+H]+: 356.1

### Synthesis of rac-7-bromo-4-((4-methoxybenzyl)oxy)-2-((1S*,2S*)-2-(4-methylpyrimidin-2-yl)cyclopropyl)quinoline

To a mixture of rac-7-bromo-2-((1S*,2S*)-2-(4-methylpyrimidin-2-yl)cyclopropyl)quinolin-4 (1H)-one (2.0 g, 5.6 mmol) in DMF (20 mL) was added NaH (336 mg, 60% in mineral oil, 8.4 mmol) in portionwise and the mixture was stirred at room temperature for 1 h. 1-(chloromethyl)-4-methoxybenzene (1.3 g, 8.4 mmol) was added and the resulting reaction mixture was stirred at room temperature for 1 h. The reaction was quenched with NH₄Cl (sat. aq., 100 mL) and extracted with EtOAc (100 mL×3). The combined organic layers were washed with brine (100 mL), dried over Na₂SO₄ then concentrated to give the crude, which was purified by column chromatography (eluent: DCM/MeOH=0-5%) to give rac-7-bromo-4-((4-methoxybenzyl)oxy)-2-((1S*,2S*)-2-(4-methylpyrimidin-2-yl)cyclopropyl)quinoline (2.0 g, 75%) as yellow solid. ESI-MS [M+H]+: 476.2

### Synthesis of rac-7-bromo-4-((4-methoxybenzyl)oxy)-2-((1S*,2S*)-2-(4-methylpyrimidin-2-yl)cyclopropyl)quinoline

To a mixture of rac-7-bromo-4-((4-methoxybenzyl)oxy)-2-((1S*,2S*)-2-(4-methylpyrimidin-2-yl)cyclopropyl)quinoline (2.0 g, 4.2 mmol), tert-butyl carbamate (737 mg, 6.3 mmol) and Cs₂CO₃ (4.1 g, 12.6 mmol) in dioxane (20 mL) was added Pd₂(dba)₃ (384 mg, 0.42 mmol) and xantPhos (364 mg, 0.63 mmol). The resulting reaction mixture was stirred at 95° C. for 18 h under N₂. After cooling to room temperature, the reaction mixture was filtered through Celite® and the filter cake was washed with DCM/MeOH (10/1, 100 mL). The filtrate was concentrated in vacuo to give the crude, which was purified by column chromatography (eluent: DCM/MeOH=0˜5%) to give rac-tert-butyl (4-((4-methoxybenzyl)oxy)-2-((1S*,2S*)-2-(4-methylpyrimidin-2-yl)cyclopropyl)quinolin-7-yl)carbamate (1.2 g, 56%) as a gray solid. ESI-MS [M+H]⁺: 513.2

### Synthesis of rac-tert-butyl (4-hydroxy-2-((1S*,2S*)-2-(4-methylpyrimidin-2-yl)cyclopropyl)quinolin-7-yl)carbamate

A mixture of rac-tert-butyl (4-((4-methoxybenzyl)oxy)-2-((1S*,2S*)-2-(4-methylpyrimidin-2-yl)cyclopropyl)quinolin-7-yl)carbamate (1.2 g, 2.3 mmol) and Pd/C (0.12 g) in MeOH (10 mL) was stirred at room temperature for 2 h under H₂. The reaction mixture was filtered through Celite® and the filter cake was washed with DCM/MeOH (V/V=10/1, 50 mL). The filtrate was concentrated in vacuo to give the crude, which was purified by column chromatography on silica gel, eluting with 0˜10% MeOH/DCM to give rac-tert-butyl (4-hydroxy-2-((1S*,2S*)-2-(4-methylpyrimidin-2-yl)cyclopropyl) quinolin-7-yl)carbamate (0.60 g, 66%) as a yellow solid. ESI-MS [M+H]+: 393.2

### Synthesis of rac-tert-butyl (4-bromo-2-((7S*,2S*)-2-(4-methylpyrimidin-2-yl)cyclopropyl)quinolin-7-yl)carbamate

To a mixture of rac-tert-butyl (4-hydroxy-2-((1S*,2S*)-2-(4-methylpyrimidin-2-yl)cyclo propyl)quinolin-7-yl)carbamate (0.60 g, 1.5 mmol) in DMF (10 mL) was added PBr₃ (0.62 g, 2.3 mmol) at 0° C. After stirring at room temperature for 5 h, the reaction mixture was quenched with NaHCO₃ (sat. aq., 100 mL) and extracted with EtOAc (100 mL×3). The combined organic layers were washed with brine (50 mL), dried over Na₂SO₄ and concentrated in vacuo to get a residue, which was purified by column chromatography on silica gel, eluting with 0˜5% MeOH/DCM to afford rac-tert-butyl (4-bromo-2-((1S*,2S*)-2-(4-methylpyrimidin-2-yl)cyclopropyl)quinolin-7-yl)carbamate (0.30 g, 43%) as a yellow oil. ESI-MS [M+H]+: 455.1

### Synthesis of rac-4-bromo-2-((1S*,2S*)-2-(4-methylpyrimidin-2-yl)cyclopropyl)quinolin-7-amine

To a solution of rac-tert-butyl (4-bromo-2-((1S*,2S*)-2-(4-methylpyrimidin-2-yl)cyclo propyl)quinolin-7-yl)carbamate (0.40 g, 0.88 mmol) in DCM (5.0 mL) was added TFA (0.5 mL) at 0° C. After stirring at room temperature for 2 h, the reaction mixture was quenched with NaHCO₃ (sat. aq., 50 mL) and extracted with EtOAc (50 mL×3). The combined organic layers were washed with brine (50 mL), dried over Na₂SO₄ and then concentrated in vacuo to get a residue, which was purified by preparative TLC, eluting with 50% EtOAc/PE to give rac-4-bromo-2-((1S*,2S*)-2-(4-methyl pyrimidin-2-yl)cyclopropyl)quinolin-7-amine (0.20 g, 64%) as a yellow oil. ESI-MS [M+H]+: 355.1

### Synthesis of rac-1-(2-(((4-bromo-2-((1S*,2S*)-2-(4-methylpyrimidin-2-yl)cyclopropyl)quinolin-7-yl)amino)methyl)-6-cyclopropylimidazo[1,2-a]pyridin-8-yl)-3-methylimidazolidine-2,4-dione

A mixture of rac-4-bromo-2-((1S*,2S*)-2-(4-methylpyrimidin-2-yl)cyclopropyl)quinolin-7-amine (0.10 g, 0.28 mmol) and 6-cyclopropyl-8-(3-methyl-2,4-dioxoimidazolidin-1-yl)imidazo[1,2-a]pyridine-2-carbaldehyde (84 mg, 0.28 mmol) in THF (5.0 mL) was added Ti(O-iPr)₄ (0.40 g, 1.4 mmol). After stirred at 80° C. for 16 h, the mixture was added MeOH (0.5 mL) and NaBH₃CN (53 mg, 0.84 mmol) at room temperature. The resulting mixture was stirred room temperature for 2 h. The reaction was diluted with water (30 mL) and then extracted with EtOAc (30 mL×3). The combined organic layers were washed with brine (30 mL), dried over Na₂SO₄ and then concentrated in vacuo to get a residue, which was purified by preparative TLC, eluting with 10% MeOH/DCM to afford rac-1-(2-(((4-bromo-2-((1S*,2S*)-2-(4-methylpyrimidin-2-yl)cyclopropyl)quinolin-7-yl)amino)methyl)-6-cyclopropylimidazo[1,2-a]pyridin-8-yl)-3-methylimidazolidine-2,4-dione (80 mg, 45%) as an off-white solid. ESI-MS [M+H]+: 637.2

### Synthesis of rac-1-(6-cyclopropyl-2-(((2-((1S*,2S*)-2-(4-methylpyrimidin-2-yl)cyclopropyl)quinolin-7-yl)amino)methyl)imidazo[1,2-a]pyridin-8-yl)-5-(3-hydroxyoxetan-3-yl)-3-methylimidazolidine-2,4-dione

To a solution of rac-1-(2-(((4-bromo-2-((1S*,2S*)-2-(4-methylpyrimidin-2-yl)cyclopropyl)quinolin-7-yl)amino)methyl)-6-cyclopropylimidazo[1,2-a]pyridin-8-yl)-3-methyl imidazolidine-2,4-dione (80 mg, 0.13 mmol) in THF (5 mL) was added butyllithium (0.16 mL, 0.38 mmol, 2.4 M in hexane) at −78° C. After stirring at −78° C. for 10 min, Oxetan-3-one (45 mg, 0.63 mmol) was added and the resulting mixture was stirred at −78° C. for another 1 h. The reaction was quenched with NH₄Cl (sat. aq., 50 mL) and extracted with EtOAc (30 mL×3). The combined organic layers were washed with brine (30 mL), dried over Na₂SO₄ and concentrated in vacuo to get a residue, which was purified by preparative TLC, eluting with 10% MeOH/DCM to give rac-1-(6-cyclopropyl-2-(((2-((1S*,2S*)-2-(4-methylpyrimidin-2-yl)cyclopropyl)quinolin-7-yl)amino)methyl)imidazo[1,2-a]pyridin-8-yl)-5-(3-hydroxyoxetan-3-yl)-3-methylimidazolidine-2,4-dione (5.0 mg, 6%) as an off-white solid.

ESI-MS [M+H]+: 631.3, ¹H NMR (400 MHz, DMSO) δ 8.51 (d, J=5.1 Hz, 1H), 8.38 (s, 1H), 7.91 (d, J=8.2 Hz, 1H), 7.80 (s, 1H), 7.58 (d, J=8.8 Hz, 1H), 7.17 (d, J=3.6 Hz, 2H), 7.11 (d, J=8.4 Hz, 1H), 7.07-7.02 (m, 1H), 6.85 (s, 1H), 6.75 (s, 1H), 5.58 (s, 1H), 4.99 (d, J=7.2 Hz, 1H), 4.54-4.47 (m, 2H), 3.85 (d, J=3.1 Hz, 1H), 3.70 (d, J=6.6 Hz, 1H), 2.96 (s, 3H), 2.71-2.64 (m, 2H), 2.41 (s, 3H), 1.97-1.92 (m, 1H), 1.73 (dd, J=24.5, 4.2 Hz, 2H), 0.95 (d, J=6.6 Hz, 2H), 0.67 (d, J=4.3 Hz, 2H).

### Example 91

### Synthesis of 6-((6-cyclopropylimidazo[1,2-a]pyridin-2-yl)methoxy)-3-((1S,2S)-2-(4-methylpyrimidin-2-yl)cyclopropyl)-4H-benzo[e][1,2,4]thiadiazine 1,1-dioxide (I-91)

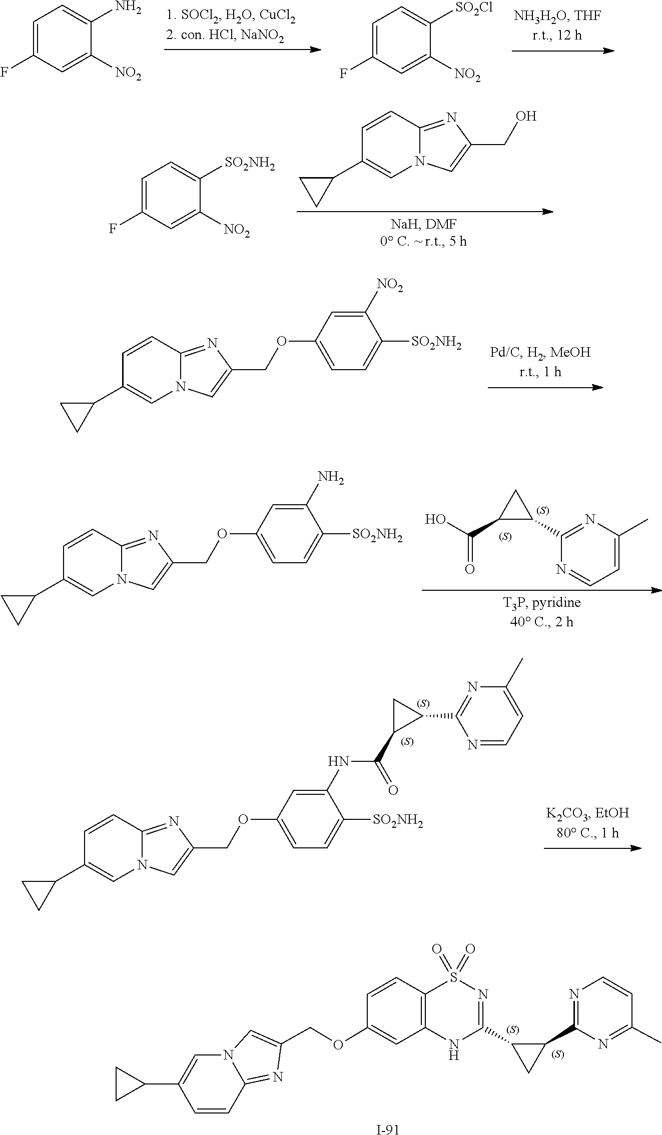

### Synthesis of 4-fluoro-2-nitrobenzenesulfonyl chloride

SOCl₂ (1.8 mL, 24.8 mmol) was added dropwise to ice water (10 mL) and the mixture was stirred at room temperature for 18 h. CuCl₂ (32 mg, 0.24 mmol) was added at 0° C. and the mixture was stirred for 15 minutes. In a separate flask, a solution of NaNO₂ (270 mg, 3.9 mmol) in water (4.5 mL) was added to a stirred solution of 4-fluoro-2-nitroaniline (437 mg, 2.8 mmol) in conc. HCl (4.5 mL) at 0° C. over 15 minutes. The resulting diazonium salt solution was added dropwise to the thionyl chloride/CuCl₂ solution at 0° C. and the resulting mixture was stirred at 0° C. for 1 h then extracted with DCM (40 mL×3). The combined organic phases were washed with brine (40 mL), dried over Na₂SO₄ and concentrated in vacuo to give 4-fluoro-2-nitrobenzenesulfonyl chloride (500 mg, 75%) as a yellow solid, which was used in the next step without purification. ESI-MS [M+H]+: 240.2.

### Synthesis of 4-fluoro-2-nitrobenzenesulfonamide

To a solution of 4-fluoro-2-nitrobenzenesulfonyl chloride (500 mg, 2.09 mmol) in THF (20 mL) was added ammonium hydroxide (1 mL, conc.). The reaction mixture was stirred at room temperature for 12 h. The reaction was concentrated in vacuo to give the crude, which was purified by silica gel column chromatography (eluent: MeOH/DCM=0-3%) to give 4-fluoro-2-nitrobenzene sulfonamide (400 mg, 87%) as a yellow solid. ESI-MS [M+H]+: 221.2.

### Synthesis of 4-((6-cyclopropylimidazo[1,2-a]pyridin-2-yl)methoxy)-2-nitrobenzenesulfonamide

To a mixture of (6-cyclopropylimidazo[1,2-a]pyridin-2-yl)methanol (300 mg, 1.6 mmol) in DMF (5 mL) was added NaH (256 mg, 6.4 mmol, 60% dispersion in mineral oil) at 0° C. under N₂. The mixture was stirred at 0° C. for 1 h, then a solution of 4-fluoro-2-nitrobenzenesulfonamide (352 mg, 1.6 mmol) in DMF (2 mL) was added thereto. The reaction mixture was allowed to warm to room temperature and stirred for 5 h. The reaction was quenched with water (100 mL) and extracted with EtOAc (40 mL×3). The combined organic layers were washed with brine (30 mL), dried over anhydrous Na₂SO₄, and concentrated in vacuo to give the crude, which was purified by preparative TLC (eluent: DCM/MeOH=30/1) to give 4-((6-cyclopropylimidazo[1,2-a]pyridin-2-yl)methoxy)-2-nitrobenzenesulfonamide (220 mg, 35%) as a yellow solid. ESI-MS [M+H]+: 389.2.

### Synthesis of 2-amino-4-((6-cyclopropylimidazo[1,2-a]pyridin-2-yl)methoxy)benzenesulfonamide

A mixture of 4-((6-cyclopropylimidazo[1,2-a]pyridin-2-yl)methoxy)-2-nitrobenzene sulfonamide (220 mg, 0.57 mmol) and Pd/C (50 mg) in MeOH (10 mL) was stirred at room temperature for 1 h under and atmosphere of H₂. The reaction mixture was filtered through Celite® and the filter cake was washed with MeOH (50 mL). The filtrate was concentrated in vacuo to give the crude, which was purified by preparative TLC (eluent: DCM/MeOH=10/1) to give 2-amino-4-((6-cyclopropylimidazo[1,2-a]pyridin-2-yl)methoxy)benzenesulfonamide (120 mg, 59%) as a yellow solid.

ESI-MS [M+H]+: 359.2.

### Synthesis of (1S,2S)—N-(5-((6-cyclopropylimidazo[1,2-a]pyridin-2-yl)methoxy)-2-sulfamoylphenyl)-2-(4-methylpyrimidin-2-yl)cyclopropane-1-carboxamide

A mixture of 2-amino-4-((6-cyclopropylimidazo[1,2-a]pyridin-2-yl)methoxy)benzenesulfonamide (60 mg, 0.17 mmol), (1S,2S)-2-(4-methylpyrimidin-2-yl)cyclo propane-1-carboxylic acid (30 mg, 0.17 mmol) and T₃P (1.1 g, 1.7 mmol, 50% solution in EtOAc) in pyridine (2 mL) was stirred at 40° C. for 2 h. Water (20 mL) was added and extracted with EtOAc (30 mL×3). The combined organic layers were washed with brine (30 mL), dried over Na₂SO₄, filtered and concentrated in vacuo to give the crude, which was purified by preparative TLC (eluent: DCM/MeOH=20/1) to give (1S,2S)—N-(5-((6-cyclopropylimidazo[1,2-a]pyridin-2-yl)methoxy)-2-sulfamoylphenyl)-2-(4-methylpyrimidin-2-yl)cyclopropane-1-carboxamide (40 mg, 45%) as a white solid. ESI-MS [M+H]+: 519.2

### Synthesis of 6-((6-cyclopropylimidazo[1,2-a]pyridin-2-yl)methoxy)-3-((1S,2S)-2-(4-methylpyrimidin-2-yl)cyclopropyl)-4H-benzo[e][1,2,4]thiadiazine 1,1-dioxide

To a solution of (1S,2S)—N-(5-((6-cyclopropylimidazo[1,2-a]pyridin-2-yl)methoxy)-2-sulfamoylphenyl)-2-(4-methylpyrimidin-2-yl)cyclopropane-1-carboxamide (40 mg, 0.077 mmol), in EtOH (4 mL) was added K₂CO₃ (32 mg, 0.23 mmol). The reaction mixture was stirred at 80° C. for 1 h. The reaction was cooled to room temperature, water (20 mL) was added and extracted with EtOAc (20 mL×3). The combined organic layers were washed with brine (30 mL), dried over anhydrous Na₂SO₄, and concentrated in vacuo to give the crude, which was purified with preparative TLC (eluent: DCM/MeOH=10/1) to give 6-((6-cyclopropylimidazo[1,2-a]pyridin-2-yl)methoxy)-3-((1S,2S)-2-(4-methylpyrimidin-2-yl)cyclopropyl)-4H-benzo[e][1,2,4]thiadiazine 1,1-dioxide (19 mg, 49%) as a white solid.

ESI-MS [M+H]+: 501.2, ¹H NMR (400 MHz, DMSO) δ 12.31 (s, 1H), 8.56 (d, J=5.1 Hz, 1H), 8.34 (s, 1H), 7.87 (s, 1H), 7.69 (d, J=8.9 Hz, 1H), 7.43 (d, J=9.3 Hz, 1H), 7.26 (d, J=5.1 Hz, 1H), 7.14-7.10 (m, 1H), 7.03-6.98 (m, 1H), 6.83 (d, J=2.3 Hz, 1H), 5.27 (s, 2H), 2.70-2.64 (m, 1H), 2.52-2.50 (m, 1H), 2.44 (s, 3H), 1.77-1.70 (m, 1H), 1.69-1.65 (m, 2H), 0.99-0.87 (m, 2H), 0.75-0.62 (m, 2H).

### Example 92

### Synthesis of rac-6-((6-cyclopropylimidazo[1,2-a]pyridin-2-yl)methoxy)-3-((1S*,2S*)-2-(4-methylpyrimidin-2-yl)cyclopropyl)-4H-benzo[e][1,2,4]thiadiazine 1,1-dioxide (I-92)

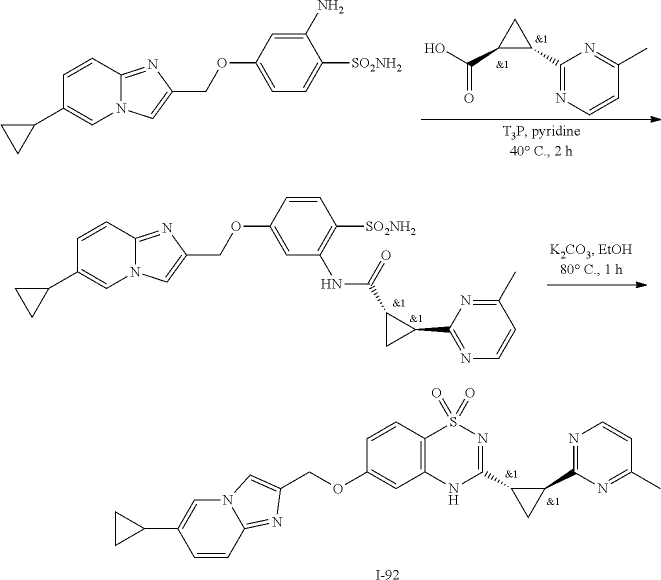

### Synthesis of rac-(1S*,2S*)—N-(5-((6-cyclopropylimidazo[1,2-a]pyridin-2-yl)methoxy)-2-sulfamoylphenyl)-2-(4-methylpyrimidin-2-yl)cyclopropane-1-carboxamide

A mixture of 2-amino-4-((6-cyclopropylimidazo[1,2-a]pyridin-2-yl)methoxy)benzenesulfonamide (120 mg, 0.34 mmol), rac-(1S*,2S*)-2-(4-methylpyrimidin-2-yl)cyclopropane-1-carboxylic acid (60 mg, 0.34 mmol), T₃P (2.2 g, 3.4 mmol, 50% solution in EtOAc) in pyridine (5 mL) was stirred at 40° C. for 2 h. The reaction was quenched with water (30 mL) and extracted with EtOAc (40 mL×3). The combined organic layers were washed with brine (30 mL), dried over Na₂SO₄, filtered and concentrated in vacuo to give the crude, which was purified by preparative TLC (eluent: DCM/MeOH=20/1) to give rac-(1S*,2S*)—N-(5-((6-cyclopropylimidazo[1,2-a]pyridin-2-yl)methoxy)-2-sulfamoylphenyl)-2-(4-methylpyrimidin-2-yl)cyclopropane-1-carboxamide (85 mg, 48%) as a white solid. ESI-MS [M+H]+: 519.2

### Synthesis of rac-6-((6-cyclopropylimidazo[1,2-a]pyridin-2-yl)methoxy)-3-((1S*,2S*)-2-(4-methylpyrimidin-2-yl)cyclopropyl)-4H-benzo[e][1,2,4]thiadiazine 1,1-dioxide

To a solution of rac-(1S*,2S*)—N-(5-((6-cyclopropylimidazo[1,2-a]pyridin-2-yl)methoxy)-2-sulfamoylphenyl)-2-(4-methylpyrimidin-2-yl)cyclopropane-1-carboxamide (85 mg, 0.16 mmol) in EtOH (8 mL) was added K₂CO₃ (55 mg, 0.4 mmol). The reaction mixture was stirred at 80° C. for 1 h. The reaction was cooled to room temperature and quenched with H₂O (30 mL), extracted with EtOAc (30 mL×3). The combined organic layers were washed with brine (30 mL), dried over anhydrous Na₂SO₄, and concentrated in vacuo to give crude, which was purified with preparative TLC (eluent: DCM/MeOH=10/1) to give rac-6-((6-cyclopropylimidazo[1,2-a]pyridin-2-yl)methoxy)-3-((1S*,2S*)-2-(4-methylpyrimidin-2-yl)cyclopropyl)-4H-benzo[e][1,2,4]thiadiazine 1,1-dioxide (25 mg, 31%) as a white solid.

ESI-MS [M+H]+: 501.1. ¹H NMR (400 MHz, DMSO) δ 12.31 (s, 1H), 8.56 (d, J=5.1 Hz, 1H), 8.34 (s, 1H), 7.87 (s, 1H), 7.69 (d, J=8.9 Hz, 1H), 7.43 (d, J=9.3 Hz, 1H), 7.26 (d, J=5.1 Hz, 1H), 7.14-7.10 (m, 1H), 7.03-6.98 (m, 1H), 6.83 (d, J=2.3 Hz, 1H), 5.27 (s, 2H), 2.70-2.64 (m, 1H), 2.52-2.50 (m, 1H), 2.44 (s, 3H), 1.77-1.70 (m, 1H), 1.69-1.65 (m, 2H), 0.99-0.87 (m, 2H), 0.75-0.62 (m, 2H).

### Example 93

### Synthesis of 4-chloro-2-((1S,2S)-2-(7-(((6-cyclopropyl-8-(3-methyl-2,4-dioxoimidazolidin-1-yl)imidazo[1,2-a]pyridin-2-yl)methyl)amino)quinolin-2-yl)cyclopropyl)benzonitrile (I-93)

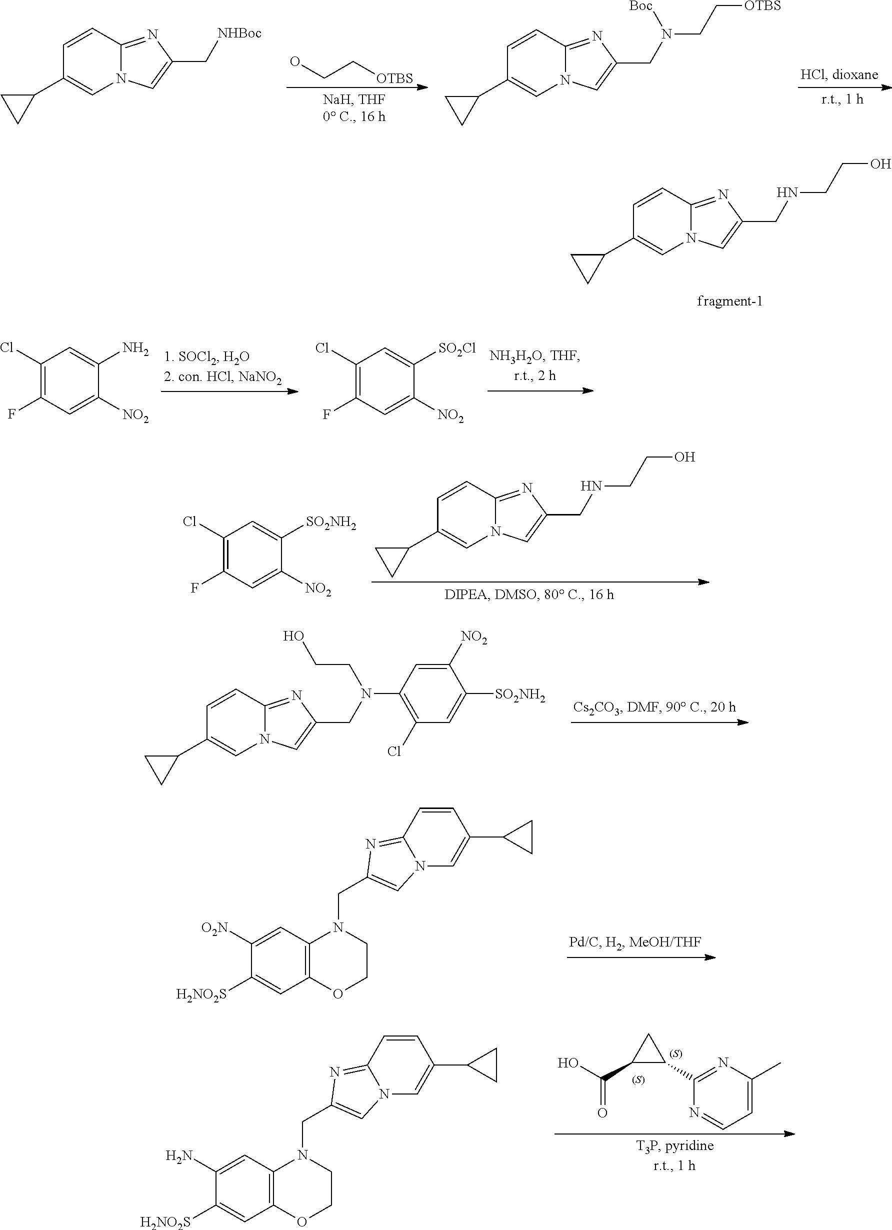

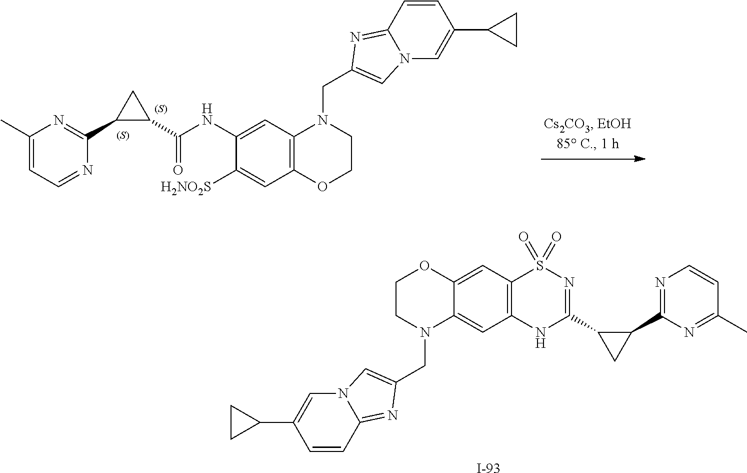

### Synthesis of tert-butyl (2-((tert-butyldimethylsilyl)oxy)ethyl)((6-cyclopropylimidazo[1,2-a]pyridin-2-yl)methyl)carbamate

To a solution of tert-butyl ((6-cyclopropylimidazo[1,2-a]pyridin-2-yl)methyl)carbamate (1.44 g, 5 mmol) in anhydrous THF (30 mL) was added NaH (300 mg, 7.5 mmol, 60% dispersion in mineral oil) at 0° C. and the mixture was warm to room temperature and stirred for 30 min. After the reaction was cooled to 0° C., a solution of tert-butyl(2-bromoethoxy)dimethylsilane (3.57 g, 15 mmol) in THF (5 mL) was added dropwise over 5 min. The resulting mixture was slowly warmed to room temperature and stirred for 16 h. Then the reaction was quenched with NH₄Cl (sat. aq, 80 mL) at 0° C. and extracted with EtOAc (60 mL×3). The combined organic layers were washed with brine (80 mL), dried over NaSO4, filtered and concentrated in vacuo to give the crude, which was purified with silica gel chromatography (eluent: MeOH/DCM=0˜3%) to give tert-butyl (2-((tert-butyldimethylsilyl) oxy)ethyl)((6-cyclopropylimidazo[1,2-a]pyridin-2-yl)methyl)carbamate (1.2 g, 54%) as yellow oil.

ESI-MS [M+H]⁺: 446.3.

### Synthesis of 2-(((6-cyclopropylimidazo[1,2-a]pyridin-2-yl)methyl)amino)ethan-1-ol

To a solution of the tert-butyl (2-((tert-butyldimethylsilyl)oxy)ethyl)((6-cyclopropyl imidazo[1,2-a]pyridin-2-yl)methyl)carbamate (1.2 g, 2.7 mol) in dioxane (10 mL) was added HCl (4 M solution in dioxane, 5 mL) and the reaction was stirred at room temperature for 1 h. The reaction was concentrated in vacuo and the residue was neutralized with NaHCO₃ (sat. aq., 40 mL) and extracted with DCM (40 mL×3). The combined organic layers were washed with brine (50 mL), dried over Na₂SO₄, concentrated in vacuo to give the crude, which was purified by silica gel chromatography (eluent: MeOH/DCM=0˜10%) to afford 2-(((6-cyclopropylimidazo[1,2-a]pyridin-2-yl)methyl)amino)ethan-1-ol (500 mg, 80%) as a yellow solid. ESI-MS [M+H]⁺: 232.1.

### Synthesis of 5-chloro-4-fluoro-2-nitrobenzenesulfonyl chloride

SOCl₂ (23.9 g, 201 mmol) was added dropwise to ice water (200 mL) and stirred for 16 h at room temperature, CuCl₂ (241 mg, 1.8 mmol) was added at 0° C. and the mixture was stirred at 0° C. for 15 minutes. A solution of NaNO₂ (3.79 g, 54.9 mmol) in water (40 ml) was added to a stirred solution of 5-chloro-4-fluoro-2-nitroaniline (7 g, 36.7 mmol) in conc. HCl (40 mL) at 0° C. over 15 minutes. The resulting diazonium salt solution was added dropwise to the above prepared SOCl₂/CuCl₂ solution at 0° C. and the resulting mixture was stirred 0° C. for 1 h. The reaction mixture was extracted with DCM (100 mL×3). The combined organic layers were washed with brine (50 mL), dried over Na₂SO₄, concentrated in vacuo to give the crude, which was purified by column chromatography (eluent: EtOAc/PE=0˜10%) to give the 5-chloro-4-fluoro-2-nitrobenzenesulfonyl chloride (3.2 g, 32%) as yellow solid. ESI-MS [M+H]⁺: 274.0.

### Synthesis of 5-chloro-4-fluoro-2-nitrobenzenesulfonamide

To a stirred mixture of 5-chloro-4-fluoro-2-nitrobenzenesulfonyl chloride (3.2 g, 11.7 mmol) in THF (30 mL) was added NH₄OH (10 mL, conc.) at 0° C. After the reaction mixture was stirred at room temperature for 2 h, the reaction was concentrated in vacuo to give the crude, which was purified by column gel chromatography (eluent: MeOH/DCM=0˜1%) to give 5-chloro-4-fluoro-2-nitrobenzenesulfonamide (2.5 g, 84%) as a yellow solid. ESI-MS [M+Na]⁺: 277.0.

### Synthesis of 5-chloro-4-(((6-cyclopropylimidazo[1,2-a]pyridin-2-yl)methyl)(2-hydroxyethyl)amino)-2-nitrobenzenesulfonamide

To a mixture of 5-chloro-4-fluoro-2-nitrobenzenesulfonamide (1.0 g, 4 mmol) and 2-(((6-cyclopropylimidazo[1,2-a]pyridin-2-yl)methyl)amino)ethan-1-ol (464 mg, 2 mmol) in DMSO (20 mL) was added DIPEA (770 mg, 6 mmol). The reaction was stirred at 80° C. for 16 h. The mixture was quenched with NaHCO₃ (sat. aq., 100 mL) and extracted with EtOAc (100 mL×3). The combined organic layers were washed with brine (50 mL), dried over Na₂SO₄ and concentrated in vacuo. The residue was purified by column chromatography (eluent: MeOH/DCM=0˜5%) to give the 5-chloro-4-(((6-cyclopropylimidazo[1,2-a]pyridin-2-yl)methyl)(2-hydroxyethyl)amino)-2-nitrobenzene sulfonamide (400 mg, 43%) as a yellow solid. ESI-MS [M+H]⁺: 466.1.

### Synthesis of 4-((6-cyclopropylimidazo[1,2-a]pyridin-2-yl)methyl)-6-nitro-3,4-dihydro-2H-benzo[b][1,4]oxazine-7-sulfonamide

To a stirred solution of 5-chloro-4-(((6-cyclopropylimidazo[1,2-a]pyridin-2-yl)methyl)(2-hydroxyethyl)amino)-2-nitrobenzenesulfonamide (350 mg, 0.75 mmol) in DMF (30 mL) was added Cs₂CO₃ (734 mg, 2.25 mol) and the reaction was stirred at 90° C. for 20 h. After cooling to room temperature, the mixture was quenched with brine (sat. aq., 100 mL) and extracted with EtOAc (3×50 mL). The combined organic layers were washed with brine (50 mL), dried over Na₂SO₄ and concentrated in vacuo to give the crude, which was purified by column chromatography (eluent: MeOH/DCM=0˜3%) to give the 4-((6-cyclopropylimidazo[1,2-a]pyridin-2-yl)methyl)-6-nitro-3,4-dihydro-2H-benzo[b][1,4]oxazine-7-sulfonamide (115 mg, 36%) as a yellow solid. ESI-MS [M+H]+: 430.1.

### Synthesis of 6-amino-4-((6-cyclopropylimidazo[1,2-a]pyridin-2-yl)methyl)-3,4-dihydro-2H-benzo[b][1,4]oxazine-7-sulfonamide

A reaction mixture of 4-((6-cyclopropylimidazo[1,2-a]pyridin-2-yl)methyl)-6-nitro-3,4-dihydro-2H-benzo[b][1,4]oxazine-7-sulfonamide (115 mg, 0.27 mmol) and Pd/C (60 mg) in MeOH/THF (10 mL/10 mL) was stirred at room temperature for 1 h under H₂. The reaction mixture was filtered and concentrated in vacuo to give the 6-amino-4-((6-cyclopropylimidazo[1,2-a]pyridin-2-yl)methyl)-3,4-dihydro-2H-benzo[b][1,4]oxazine-7-sulfonamide (100 mg, 93%) as yellow solid. ESI-MS [M+H]+: 400.1.

### Synthesis of (1S,2S)—N-(4-((6-cyclopropylimidazo[1,2-a]pyridin-2-yl)methyl)-7-sulfamoyl-3,4-dihydro-2H-benzo[b][1,4]oxazin-6-yl)-2-(4-methylpyrimidin-2-yl)cyclopropane-1-carboxamide

To a mixture of 6-amino-4-((6-cyclopropylimidazo[1,2-a]pyridin-2-yl)methyl)-3,4-dihydro-2H-benzo[b][1,4]oxazine-7-sulfonamide (40 mg, 0.1 mmol) and (1S,2S)-2-(4-methylpyrimidin-2-yl)cyclopropane-1-carboxylic acid (21 mg, 0.12 mmol) in pyridine (3 mL) was added T3P (318 mg, 0.5 mmol, 50% in EtOAc), The reaction was stirred at room temperature for 1 h. The mixture was quenched with water (50 mL) and extracted with EtOAc (30 mL×3). The combined organic layers were washed with brine (30 mL), dried over Na₂SO₄ and concentrated in vacuo. The residue was purified by preparative TLC (eluent: DCM/MeOH=20/1) to give the (1S,2S)—N-(4-((6-cyclopropylimidazo[1,2-a]pyridin-2-yl)methyl)-7-sulfamoyl-3,4-dihydro-2H-benzo[b][1,4]oxazin-6-yl)-2-(4-methylpyrimidin-2-yl)cyclopropane-1-carboxamide (28 mg, 50%) as a yellow solid. ESI-MS [M+H]+: 560.2.

### Synthesis of 2-(2-(7-aminoquinolin-2-yl)cyclopropyl)-4-chlorobenzonitrile

To a stirred solution of (1S,2S)—N-(4-((6-cyclopropylimidazo[1,2-a]pyridin-2-yl)methyl)-7-sulfamoyl-3,4-dihydro-2H-benzo[b][1,4]oxazin-6-yl)-2-(4-methylpyrimidin-2-yl)cyclopropane-1-carboxamide (28 mg, 0.05 mmol) in EtOH (5 mL) was added Cs₂CO₃ (49 mg, 0.15 mmol). The reaction was stirred at 80° C. for 1 h. After cooling to room temperature, the reaction mixture was filtered through Celite® and the filter cake was washed with DCM/MeOH (10/1, 30 mL). The filtrate was concentrated in vacuo to give the crude, which was purified by preparative TLC (eluent: DCM/MeOH=20/1) to give the 6-((6-cyclopropylimidazo[1,2-a]pyridin-2-yl)methyl)-3-((1S,2S)-2-(4-methyl pyrimidin-2-yl)cyclopropyl)-7,8-dihydro-4H,6H-[1,4]oxazino[2′,3′:4,5]benzo[1,2-e][1,2,4]thiadiazine 1,1-dioxide (20 mg, 74%) as white solid.

ESI-MS [M+H]+: 542.2, ¹H NMR (400 MHz, DMSO) δ 11.93 (s, 1H), 8.54 (d, J=5.1 Hz, 1H), 8.25 (s, 1H), 7.65 (s, 1H), 7.39 (d, J=9.3 Hz, 1H), 7.22 (d, J=5.1 Hz, 1H), 6.98-6.96 (m, 1H), 6.90 (s, 1H), 6.47 (s, 1H), 4.65-4.56 (m, 2H), 4.25 (t, J=4.2 Hz, 2H), 3.63-3.59 (m, 2H), 2.67-2.59 (m, 1H), 2.42 (s, 3H), 2.38-2.33 (m, 1H), 1.92-1.86 (m, 1H), 1.69-1.62 (m, 2H), 0.92-0.87 (m, 2H), 0.66-0.62 (m, 2H).

### Example 94

### Synthesis of 6-(((6-cyclopropylimidazo[1,2-a]pyridin-2-yl)methyl)amino)-4-methyl-3-((1S,2S)-2-(4-methylpyrimidin-2-yl)cyclopropyl)-4H-benzo[e][1,2,4]thiadiazine 1,1-dioxide (I-94)

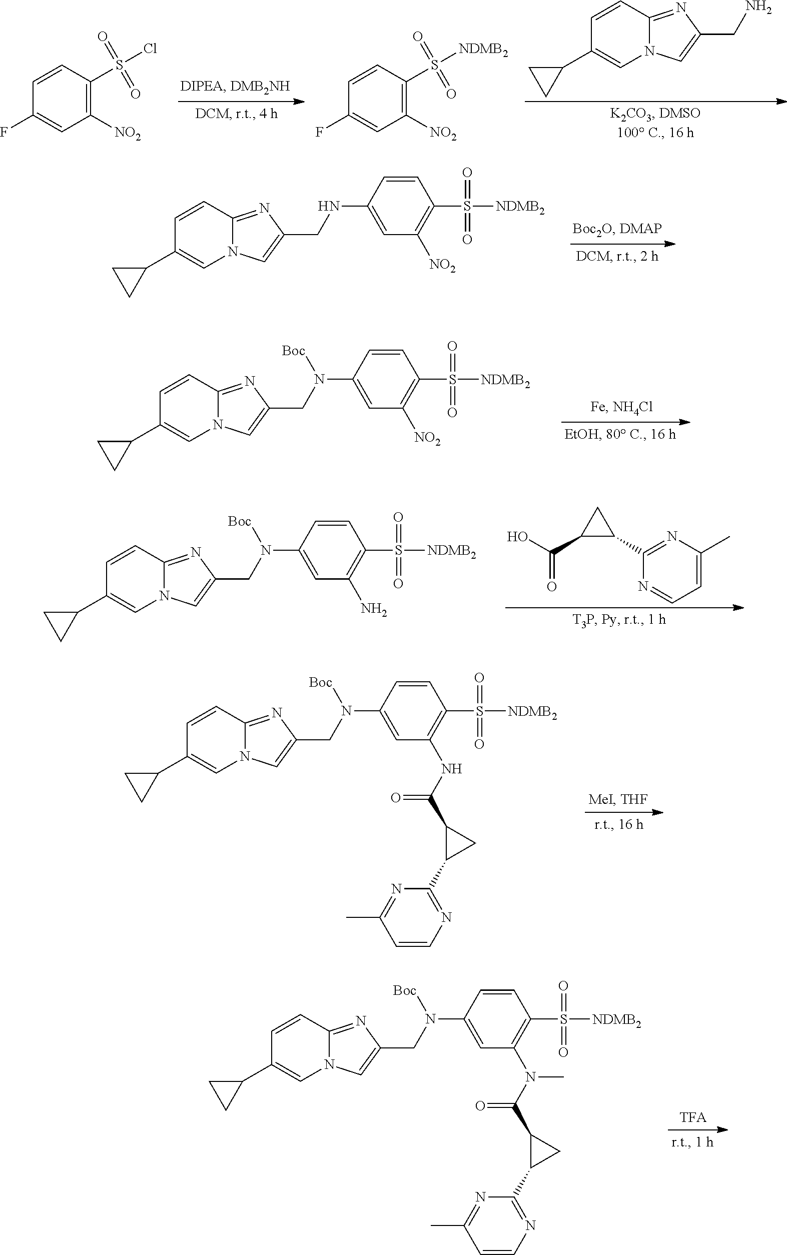

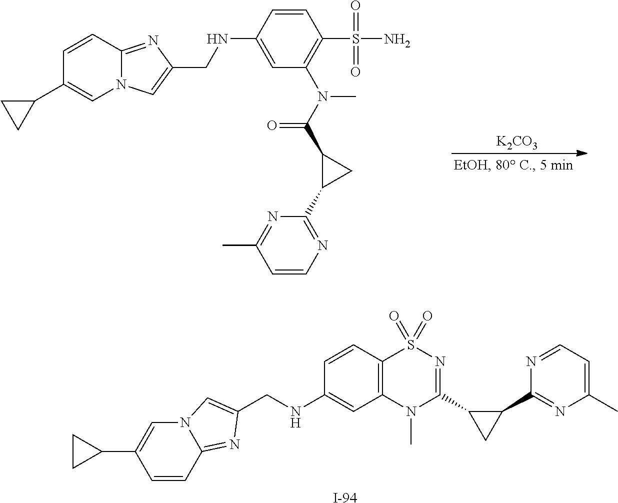

### Synthesis of N,N-bis(2,4-dimethoxybenzyl)-4-fluoro-2-nitrobenzenesulfonamide

To a mixture of 4-fluoro-2-nitrobenzenesulfonyl chloride (1 g, 4.2 mmol) and DIPEA (1.08 g, 8.4 mmol) in DCM (10 mL) was added bis(2,4-dimethoxybenzyl)amine (1.33 g, 4.18 mmol). The mixture was stirred at room temperature for 4 h. The reaction mixture was concentrated in vacuo to give the crude product, which was purified by column chromatography (eluent: DCM/MeOH=50/1) to give N,N-bis(2,4-dimethoxybenzyl)-4-fluoro-2-nitrobenzenesulfonamide (1.5 g, 65%) as a yellow solid.

ESI-MS [M+Na]+: 543.1.

### Synthesis of 4-(((6-cyclopropylimidazo[1,2-a]pyridin-2-yl)methyl)amino)-N,N-bis(2,4-dimethoxybenzyl)-2-nitrobenzenesulfonamide

A mixture of N,N-bis(2,4-dimethoxybenzyl)-4-fluoro-2-nitrobenzenesulfonamide (520 mg, 0.96 mmol), (6-cyclopropylimidazo[1,2-a]pyridin-2-yl)methanamine (198 mg, 1.06 mmol) and K₂CO₃ (265 mg, 1.92 mmol) in DMSO (10 mL) was stirred at 100° C. for 16 h under N₂. After cooling to room temperature, water (50 mL) was added and the mixture was extracted with EtOAc (30 mL×3). The combined organic layers were washed with brine (30 mL), dried over anhydrous Na₂SO₄ and concentrated in vacuo to give the crude product, which was purified by column chromatography (eluent: DCM/MeOH=100/1 to 50/1) to give 4-(((6-cyclopropylimidazo[1,2-a]pyridin-2-yl)methyl)amino)-N,N-bis(2,4-dimethoxybenzyl)-2-nitrobenzenesulfonamide (300 mg, 45%) as a yellow solid. ESI-MS [M+H]+: 688.3.

### Synthesis of tert-butyl (4-(N,N-bis(2,4-dimethoxybenzyl)sulfamoyl)-3-nitrophenyl)((6-cyclopropylimidazo[1,2-a]pyridin-2-yl)methyl)carbamate

A mixture of 4-(((6-cyclopropylimidazo[1,2-a]pyridin-2-yl)methyl)amino)-N,N-bis(2,4-dimethoxybenzyl)-2-nitrobenzenesulfonamide (300 mg, 0.44 mmol), di-tert-butyl dicarbonate (480 mg, 2.2 mmol), DMAP (54 mg, 0.44 mmol) and TEA (133 mg, 1.32 mmol) in DCM (20 mL) was stirred at room temperature for 2 h under N₂. The mixture was diluted with DCM (30 mL), washed with brine (20 mL), dried over anhydrous Na₂SO₄, and concentrated in vacuo to give the crude product, which was purified by preparative TLC (eluent: PE/EtOAc=1/1) to give tert-butyl (4-(N,N-bis(2,4-dimethoxybenzyl)sulfamoyl)-3-nitrophenyl)((6-cyclopropylimidazo[1,2-a]pyridin-2-yl)methyl)carbamate (300 mg, 87%) as a yellow solid. ESI-MS [M+H]⁺: 788.2.

### Synthesis of tert-butyl (3-amino-4-(N,N-bis(2,4-dimethoxybenzyl)sulfamoyl)phenyl)((6-cyclopropylimidazo[1,2-a]pyridin-2-yl)methyl)carbamate

A mixture of tert-butyl (4-(N,N-bis(2,4-dimethoxybenzyl)sulfamoyl)-3-nitrophenyl)((6-cyclopropylimidazo[1,2-a]pyridin-2-yl)methyl)carbamate (300 mg, 0.38 mmol) and NH₄Cl (222 mg, 4.18 mmol) in EtOH (5 mL) was heated to 80° C., then Fe (64 mg, 1.14 mmol) was added under N₂. The mixture was stirred at 80° C. for 16 h under N₂. After cooling to room temperature, the reaction mixture was filtered through Celite® and the filter cake was washed with DCM/MeOH (10/1, 50 mL). The filtrate was concentrated in vacuo to give the crude, which was purified by preparative TLC (eluent: DCM/MeOH=30/1) to give tert-butyl (3-amino-4-(N,N-bis(2,4-dimethoxybenzyl)sulfamoyl)phenyl)((6-cyclopropylimidazo[1,2-a]pyridin-2-yl)methyl)carbamate (200 mg, 69%) as a yellow solid. ESI-MS [M+H]+: 758.3.

### Synthesis of tert-butyl (4-(N,N-bis(2,4-dimethoxybenzyl)sulfamoyl)-3-((1S,2S)-2-(4-methylpyrimidin-2-yl)cyclopropane-1-carboxamido)phenyl)((6-cyclopropylimidazo[1,2-a]pyridin-2-yl)methyl)carbamate

To a mixture of tert-butyl (3-amino-4-(N,N-bis(2,4-dimethoxybenzyl)sulfamoyl)phenyl)((6-cyclopropylimidazo[1,2-a]pyridin-2-yl)methyl)carbamate (200 mg, 0.26 mmol) and (1S,2S)-2-(4-methylpyrimidin-2-yl)cyclopropane-1-carboxylic acid (70 mg, 0.39 mmol) in pyridine (3 mL) was added T₃P (1.65 g, 2.6 mmol, 50% in EtOAc) dropwise at room temperature under N₂, then the mixture was stirred at room temperature for 1 h. Then the reaction mixture was quenched with water (50 mL) and extracted with EtOAc (30 mL×3). The combined organic layers were washed with brine (50 mL), dried over anhydrous Na₂SO₄ and concentrated (1S,2S)-2-(4-methylpyrimidin-2-yl)cyclopropane-1-carboxylic acid to give the crude product, which was purified by preparative TLC (eluent: DCM/MeOH=10/1) to give tert-butyl (4-(N,N-bis(2,4-dimethoxybenzyl)sulfamoyl)-3-((1S,2S)-2-(4-methylpyrimidin-2-yl)cyclopropane-1-carboxamido)phenyl)((6-cyclopropylimidazo[1,2-a]pyridin-2-yl)methyl)carbamate (200 mg, 84%) as a yellow solid. ESI-MS [M+H]+: 918.3.

### Synthesis of tert-butyl (4-(N,N-bis(2,4-dimethoxybenzyl)sulfamoyl)-3-((1S,2S)—N-methyl-2-(4-methylpyrimidin-2-yl)cyclopropane-1-carboxamido)phenyl)((6-cyclopropylimidazo[1,2-a]pyridin-2-yl)methyl)carbamate

To a mixture of tert-butyl (4-(N,N-bis(2,4-dimethoxybenzyl)sulfamoyl)-3-((1S,2S)-2-(4-methylpyrimidin-2-yl)cyclopropane-1-carboxamido)phenyl)((6-cyclopropylimidazo[1,2-a]pyridin-2-yl)methyl)carbamate (200 mg, 0.22 mmol) in THF (5 mL) was added NaH (13 mg, 60% in mineral oil, 0.33 mmol) at 0° C. and the mixture was stirred at 0° C. for 0.5 h. A solution of MeI (47 mg, 0.33 mmol) in THF (1 mL) was added and the mixture was stirred at room temperature for 16 h under N₂. The reaction was quenched with water (20 mL) and extracted with EtOAc (30 mL×3). The combined organic layers were washed with brine (20 mL), dried over anhydrous Na₂SO₄ and concentrated in vacuo to give the crude product, which was purified by preparative TLC (eluent: DCM/MeOH=20/1) to give tert-butyl (4-(N,N-bis(2,4-dimethoxybenzyl)sulfamoyl)-3-((1S,2S)—N-methyl-2-(4-methyl pyrimidin-2-yl)cyclopropane-1-carboxamido)phenyl)((6-cyclopropylimidazo[1,2-a]pyridin-2-yl)methyl)carbamate (180 mg, 88%) as a yellow solid. ESI-MS [M+H]+: 932.3.

### Synthesis of (1S,2S)—N-(5-(((6-cyclopropylimidazo[1,2-a]pyridin-2-yl)methyl)amino)-2-sulfamoylphenyl)-N-methyl-2-(4-methylpyrimidin-2-yl)cyclopropane-1-carboxamide

A mixture of tert-butyl (4-(N,N-bis(2,4-dimethoxybenzyl)sulfamoyl)-3-((1S,2S)—N-methyl-2-(4-methylpyrimidin-2-yl)cyclopropane-1-carboxamido)phenyl)((6-cyclopropylimidazo[1,2-a]pyridin-2-yl)methyl)carbamate (180 mg, 0.19 mmol) in TFA (2 mL) was stirred at room temperature for 1 h. After cooling to 0° C., the mixture was quenched with NaHCO₃ (sat. aq., 50 mL) and extracted with DCM (30 mL×3). The combined organic layers were washed with brine (30 mL), dried over anhydrous Na₂SO₄ and concentrated in vacuo to give (1S,2S)—N-(5-(((6-cyclopropylimidazo[1,2-a]pyridin-2-yl)methyl)amino)-2-sulfamoylphenyl)-N-methyl-2-(4-methylpyrimidin-2-yl)cyclopropane-1-carboxamide (90 mg, 89%) as a yellow solid. ESI-MS [M+H]⁺: 532.2

### Synthesis of 6-(((6-cyclopropylimidazo[1,2-a]pyridin-2-yl)methyl)amino)-4-methyl-3-((1S,2S)-2-(4-methylpyrimidin-2-yl)cyclopropyl)-4H-benzo[e][1,2,4]thiadiazine 1,1-dioxide

A mixture of (1S,2S)—N-(5-(((6-cyclopropylimidazo[1,2-a]pyridin-2-yl)methyl)amino)-2-sulfamoylphenyl)-N-methyl-2-(4-methylpyrimidin-2-yl)cyclopropane-1-carboxamide (90 mg, 0.17 mmol) and K₂CO₃ (70 mg, 0.51 mmol) in EtOH (5 mL) was stirred at 80° C. for 5 min under N₂. After cooling to room temperature, the reaction was quenched with NH₄Cl (sat. aq., 30 mL) and extracted with EtOAc (30 mL×3). The combined organic layers were washed with brine (20 mL), dried over anhydrous Na₂SO₄ and concentrated in vacuo to give the crude product, which was purified by preparative TLC (DCM/MeOH=10/1) to give 6-(((6-cyclopropylimidazo[1,2-a]pyridin-2-yl)methyl)amino)-4-methyl-3-((1S,2S)-2-(4-methylpyrimidin-2-yl)cyclopropyl)-4H-benzo[e][1,2,4]thiadiazine 1,1-dioxide (35 mg, 40%) as a white solid.

ESI-MS [M+H]⁺: 514.2. ¹H NMR (400 MHz, DMSO) δ 8.57 (d, J=5.1 Hz, 1H), 8.30 (s, 1H), 7.69 (s, 1H), 7.46 (d, J=8.7 Hz, 1H), 7.38 (d, J=9.3 Hz, 1H), 7.25 (d, J=5.1 Hz, 1H), 7.17 (t, J=5.6 Hz, 1H), 6.96 (dd, J=9.3, 1.8 Hz, 1H), 6.77 (dd, J=8.7, 1.9 Hz, 1H), 6.64 (d, J=1.8 Hz, 1H), 4.45 (d, J=5.6 Hz, 2H), 3.61 (s, 3H), 2.72 (t, J=7.3 Hz, 2H), 2.45 (s, 3H), 1.92-1.87 (m, 1H), 1.76-1.71 (m, 2H), 0.92-0.89 (m, 2H), 0.66-0.65 (m, 2H).

### Example 95

### Synthesis of rac-1-(6-cyclopropyl-2-(((4-(2-hydroxypropan-2-yl)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (I-95)

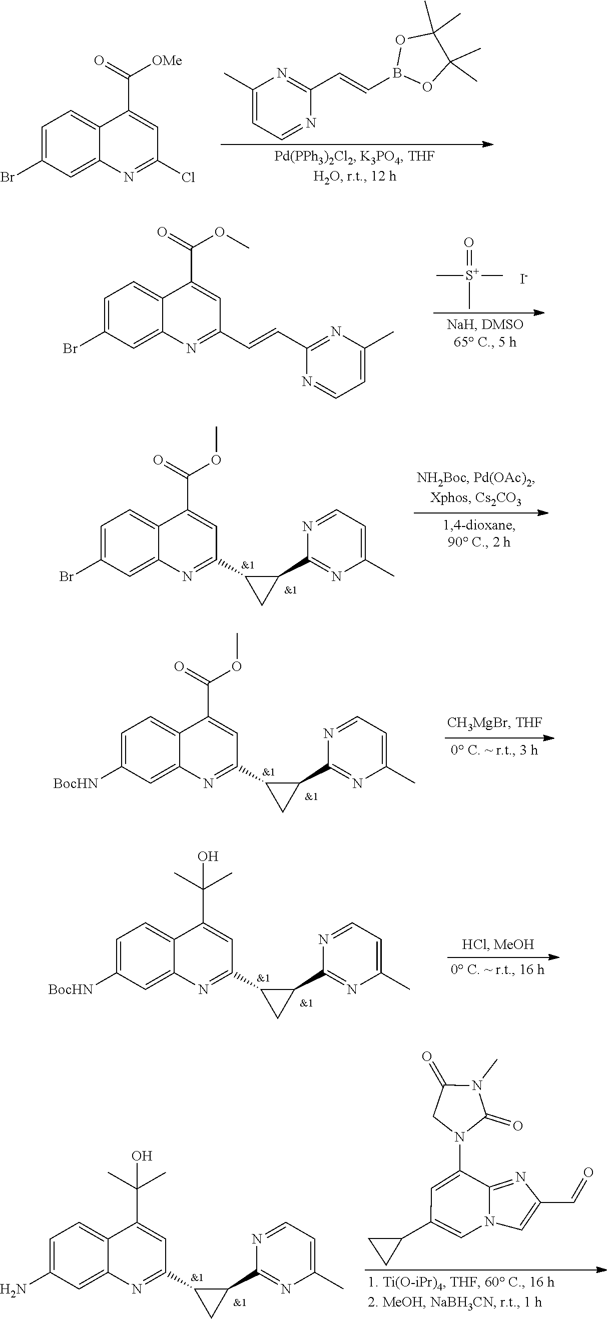

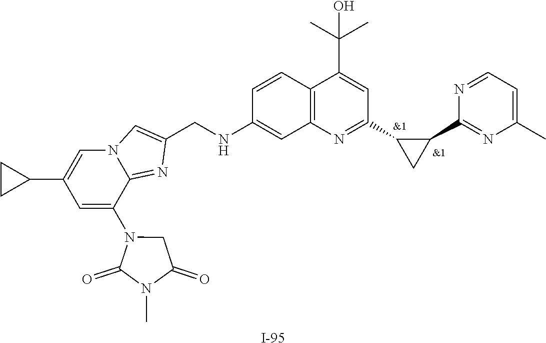

### Synthesis of methyl (E)-7-bromo-2-(2-(4-methylpyrimidin-2-yl)vinyl)quinoline-4-carboxylate

To a solution of methyl 7-bromo-2-chloroquinoline-4-carboxylate (1.7 g, 5.7 mmol) in THF/H₂O (V/V=1/1, 22 mL) was added (E)-4-methyl-2-(2-(4,4,5,5-tetramethyl-1,3,2-dioxaborolan-2-yl)vinyl)pyrimidine (2.7 g, 11 mmol), K₃PO₄ (3.4 g, 16 mmol) and Pd(PPh₃)₂Cl₂ (0.39 g, 0.56 mmol) under N₂. After the reaction was stirred at room temperature for 12 h, water (100 mL) was added and the reaction was extracted with EtOAc/MeOH (V/V=10:1, 100 mL×3). The combined organic layers were concentrated in vacuo to give a crude, which was purified by column chromatography on silica gel, eluting with 0˜30% EA/PE to give methyl (E)-7-bromo-2-(2-(4-methylpyrimidin-2-yl)vinyl)quinoline-4-carboxylate (2.0 g, 91) as a yellow solid.

ESI-MS [M+H]⁺: 384.1.

### Synthesis of rac-methyl 7-bromo-2-((1S*,2S*)-2-(4-methylpyrimidin-2-yl)cyclopropyl)quinoline-4-carboxylate

To a stirred solution of trimethylsulfoxonium iodide (5.7 g, 26 mmol) in DMSO (30 mL) was added NaH (1.0 g, 60% in mineral oil, 26 mmol) at 0° C. under N₂. After the reaction was stirred at room temperature for 1 h, a solution of methyl (E)-7-bromo-2-(2-(4-methylpyrimidin-2-yl)vinyl) quinoline-4-carboxylate (2.0 g, 5.2 mmol) in DMSO (5 mL) was added and the resulting mixture was stirred at 65° C. for another 5 h. The reaction mixture was quenched with NH₄Cl (sat. aq., 100 mL) and then extracted with EtOAc/MeOH (V/V=1/1, 100 mL×3). The combined organic layers were concentrated in vacuo to give a crude, which was purified by column chromatography on silica gel, eluting with 0˜20% EtOAc/PE to give rac-methyl 7-bromo-2-((1S*,2S*)-2-(4-methylpyrimidin-2-yl)cyclopropyl)quinoline-4-carboxylate (1.0 g, 48%) as a white solid. ESI-MS [M+H]⁺: 398.1.

### Synthesis of rac-methyl 7-((tert-butoxycarbonyl)amino)-2-((1S*,2S*)-2-(4-methylpyrimidin-2-yl)cyclopropyl)quinoline-4-carboxylate

To a solution of rac-methyl 7-bromo-2-((1S*,2S*)-2-(4-methylpyrimidin-2-yl)cyclopropyl)quinoline-4-carboxylate (1.0 g, 2.5 mmol) in 1,4-dioxane (30 mL) was added NH₂Boc (0.59 g, 5.0 mmol), Cs₂CO₃ (2.3 g, 7.1 mmol), Xantphos (0.24 g, 0.41 mmol) and Pd(OAc)₂ (0.11 g, 0.49 mmol) under N₂, the reaction mixture was stirred at 90° C. for 2 h. After the reaction was cooled to room temperature, the mixture was filtered and the filter cake was washed with 1,4-dioxane (20 mL), the filtrate was concentrated in vacuo to give a crude, which was purified column chromatography on silica gel, eluting with 0˜30% EtOAc/PE to give rac-methyl 7-((tert-butoxycarbonyl)amino)-2-((1S*,2S*)-2-(4-methylpyrimidin-2-yl)cyclopropyl)quinoline-4-carboxylate (1.0 g, 92%) as a yellow solid. ESI-MS [M+H]⁺: 435.2.

### Synthesis of rac-tert-butyl (4-(2-hydroxypropan-2-yl)-2-((1S*2S*)-2-(4-methylpyrimidin-2-yl)cyclopropyl)quinolin-7-yl)carbamate

To a solution of rac-methyl 7-((tert-butoxycarbonyl)amino)-2-((1S*,2S*)-2-(4-methylpyrimidin-2-yl)cyclopropyl)quinoline-4-carboxylate (0.60 g, 1.4 mmol) in THF (20 mL) was added CH₃MgBr (10 mL, 1 N in THF, 10 mmol) at 0° C. under N₂ and the resulting mixture was stirred at room temperature for 3 h. The reaction mixture was then quenched with NH₄Cl (sat. aq., 100 mL) and extracted with EtOAc/MeOH (V/V=10/1, 100 mL×3). The organic layers were concentrated in vacuo to give a crude, which was purified by column chromatography on silica gel, eluting with 0˜50% EtOAc/PE to give rac-tert-butyl (4-(2-hydroxypropan-2-yl)-2-((1S*,2S*)-2-(4-methylpyrimidin-2-yl)cyclopropyl)quinolin-7-yl)carbamate (0.54 g, 89%) as a white solid.

ESI-MS [M+H]⁺: 435.2.

### Synthesis of rac-2-(7-amino-2-((1S*,2S*)-2-(4-methylpyrimidin-2-yl)cyclopropyl)quinolin-4-yl)propan-2-ol

To a solution of rac-tert-butyl (4-(2-hydroxypropan-2-yl)-2-((1S*,2S*)-2-(4-methylpyrimidin-2-yl)cyclopropyl)quinolin-7-yl)carbamate (0.52 g, 1.2 mmol) in MeOH (5 mL) was added HCl (5 mL, 4 N in 1,4-dioxane, 20 mmol) at 0° C., the resulting mixture was stirred at room temperature for 16 h. The mixture was concentrated in vacuo and the residue was neutralized with NH₃ (5 mL, 7 N in MeOH), the resulting mixture was concentrated in vacuo to get a crude, which was purified by column chromatography on silica gel, eluting with DCM/MeOH from 0˜10% to give rac-2-(7-amino-2-((1S*,2S*)-2-(4-methylpyrimidin-2-yl)cyclopropyl)quinolin-4-yl)propan-2-ol (0.34 g, 84%) as a yellow solid.

ESI-MS [M+H]⁺: 335.2.

### Synthesis of rac-1-(6-cyclopropyl-2-(((4-(2-hydroxypropan-2-yl)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione

To a mixture of rac-2-(7-amino-2-((1S*,2S*)-2-(4-methylpyrimidin-2-yl)cyclopropyl)quinolin-4-yl)propan-2-ol (67 mg, 0.2 mmol), 6-cyclopropyl-8-(3-methyl-2,4-dioxoimidazolidin-1-yl)imidazo[1,2-a]pyridine-2-carbaldehyde (60 mg, 0.2 mmol) in THF (7 mL) was added Ti(OⁱPr)₄ (284 mg, 1.0 mmol) at room temperature under N₂. After the reaction was stirred at 60° C. for 16 h, the mixture was cooled to room temperature and was added NaBH₃CN (64 mg, 1.0 mmol) and MeOH (2 mL). The resulting reaction was stirred at room temperature for another 1 h then quenched with NH₄Cl (sat. aq., 20 mL) and extracted with EtOAc/MeOH (V/V=10/1, 50 mL×3). The combined organic layers were concentrated in vacuo to get a crude, which was purified by Prep-HPLC to give rac-1-(6-cyclopropyl-2-(((4-(2-hydroxypropan-2-yl)-2-((1S*,2S*)-2-(4-methylpyrimidin-2-yl)cyclopropyl)quinolin-7-yl)amino)methyl)imidazo[1,2-a]pyridin-8-yl)-3-methylimidazolidine-2,4-dione (90 mg, 73%) as a yellow solid.

ESI-MS [M+H]⁺: 617.2. ¹H NMR (400 MHz, DMSO) δ 8.51 (d, J=5.1 Hz, 1H), 8.42 (d, J=9.4 Hz, 1H), 8.25 (d, J=1.2 Hz, 1H), 7.74 (s, 1H), 7.35 (d, J=1.4 Hz, 1H), 7.17 (d, J=5.1 Hz, 1H), 7.14 (s, 1H), 7.02-6.99 (m, 1H), 6.79 (d, J=2.4 Hz, 1H), 6.71-6.68 (m, 1H), 5.31 (s, 1H), 4.95 (s, 2H), 4.48 (d, J=5.7 Hz, 2H), 2.98 (s, 3H), 2.70-2.62 (m, 2H), 2.41 (s, 3H), 1.95-1.89 (m, 1H), 1.79-1.73 (m, 1H), 1.71-1.67 (m, 1H), 1.62-1.61 (d, J=1.5 Hz, 6H), 0.96-0.88 (m, 2H), 0.68-0.60 (m, 2H).

### Example 96

### Synthesis of 3-methyl-1-(6-methyl-2-(((2-((1S,2S)-2-(4-methylpyrimidin-2-yl)cyclopropyl)-4-(2-oxa-6-azaspiro[3.3]heptan-6-yl)quinolin-7-yl)oxy)methyl)imidazo[1,2-a]pyridin-8-yl)imidazolidine-2,4-dione (I-96)

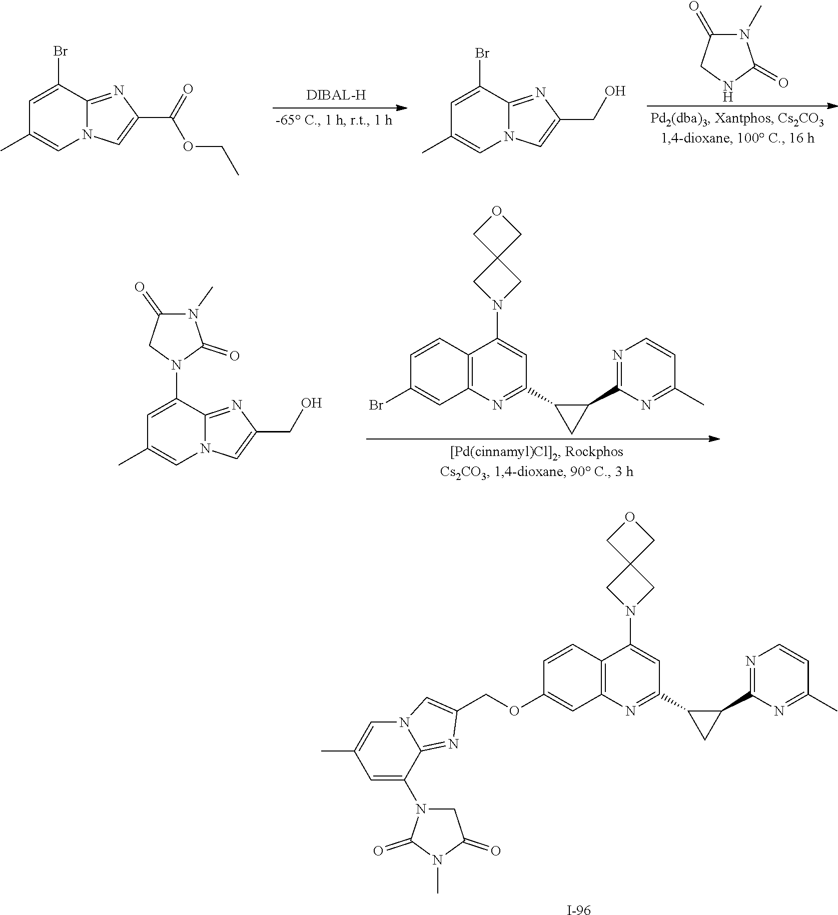

### Synthesis of (8-bromo-6-methylimidazo[1,2-a]pyridin-2-yl)methanol

To a solution of ethyl 8-bromo-6-methylimidazo[1,2-a]pyridine-2-carboxylate (prepared in a similar manner to ethyl 8-bromo-6-cyclopropyl-imidazo[1,2-a]pyridine-2-carboxylate)(1.0 g, 3.54 mmol) in THF (25 mL) was added DIBAL-H (10.5 mL, 1 M in THF, 10.5 mmol) at −65° C. under N₂ and the mixture was stirred at −65° C. for 1 h and room temperature for 1 h. The reaction mixture was quenched with water (20 mL) and filtered through Celite®, the filtrate was extracted with EtOAc (30 mL×3). The combined organics were washed with brine (40 mL) and dried over Na₂SO₄. The reaction mixture was concentrated in vacuo and purified by column chromatography (eluent: DCM/MeOH=10/1) to give (8-bromo-6-methylimidazo[1,2-a]pyridin-2-yl)methanol (440 mg, 52%) as a yellow solid.

ESI-MS [M+H]+: 241.0

### Synthesis of 1-(2-(hydroxymethyl)-6-methylimidazo[1,2-a]pyridin-8-yl)-3-methylimidazolidine-2,4-dione

To a mixture of tert-butyl (8-bromo-6-methylimidazo[1,2-a]pyridin-2-yl)methanol (440 mg, 1.82 mmol), 3-methylimidazolidine-2,4-dione (1.04 g, 9.10 mmol) and Cs₂CO₃ (1.78 g, 5.46 mmol) in 1,4-dioxane (15 mL) was added Pd₂(dba)₃ (330 mg, 0.36 mmol) and Xantphos (417 mg, 0.72 mmol). The mixture was stirred at 100° C. for 16 h under N₂. After cooling to room temperature, the reaction was filtered through Celite® and the filter cake was washed with DCM/MeOH (10/1, 40 mL). The filtrate was concentrated in vacuo to give the crude, which was purified by column chromatography (eluent: DCM/MeOH=50/1˜10/1) to give 1-(2-(hydroxymethyl)-6-methylimidazo[1,2-a]pyridin-8-yl)-3-methylimidazolidine-2,4-dione (180 mg, 36%) as a yellow solid. ESI-MS [M+H]⁺: 275.1.

### Synthesis of 3-methyl-1-(6-methyl-2-(((2-((1S,2S)-2-(4-methylpyrimidin-2-yl)cyclopropyl)-4-(2-oxa-6-azaspiro[3.3]heptan-6-yl)quinolin-7-yl)oxy)methyl)imidazo[1,2-a]pyridin-8-yl)imidazolidine-2,4-dione

To a mixture of 1-(2-(hydroxymethyl)-6-methylimidazo[1,2-a]pyridin-8-yl)-3-methylimidazolidine-2,4-dione (30 mg, 0.11 mmol), 6-(7-bromo-2-((1S,2S)-2-(4-methylpyrimidin-2-yl)cyclopropyl)quinolin-4-yl)-2-oxa-6-azaspiro[3.3]heptane (48 mg, 0.11 mmol) and Cs₂CO₃ (108 mg, 0.33 mmol) in 1,4-dioxane (5 mL) was added [Pd(cinnamyl)Cl]2 (11 mg, 0.022 mmol) and Rockphos (20 mg, 0.044 mmol). The mixture was stirred at 90° C. for 3 h under N₂. After cooling to room temperature, the reaction mixture was filtered through Celite® and the filter cake was washed with DCM/MeOH (10/1, 40 mL). The filtrate was concentrated in vacuo to give the crude, which was purified by preparative TLC (eluent: DCM/MeOH=10/1) to give 3-methyl-1-(6-methyl-2-(((2-((1S,2S)-2-(4-methylpyrimidin-2-yl)cyclopropyl)-4-(2-oxa-6-azaspiro[3.3]heptan-6-yl)quinolin-7-yl)oxy)methyl)imidazo[1,2-a]pyridin-8-yl)imidazolidine-2,4-dione (10 mg, 14%) as a yellow solid.

ESI-MS [M+H]+: 631.2. ¹H NMR (400 MHz, DMSO) δ 8.52 (d, J=5.1 Hz, 1H), 8.29 (s, 1H), 8.03 (s, 1H), 7.82 (d, J=9.0 Hz, 1H), 7.48 (d, J=1.3 Hz, 1H), 7.31 (d, J=0.6 Hz, 1H), 7.18 (d, J=5.1 Hz, 1H), 7.03 (d, J=8.9 Hz, 1H), 6.23 (s, 1H), 5.31 (s, 2H), 4.92 (s, 2H), 4.75 (s, 4H), 4.50 (s, 4H), 2.97 (s, 3H), 2.77-2.72 (m, 1H), 2.64-2.60 (m, 1H), 2.42 (s, 3H), 2.29 (s, 3H), 1.82-1.78 (m, 1H), 1.71-1.68 (m, 1H).

### Example 97

### Synthesis of 1-(6-cyclopropyl-2-(((4-((2-hydroxy-2-methylpropyl)amino)-2-((1S,2S)-2-(4-methylpyrimidin-2-yl)cyclopropyl)quinolin-7-yl)oxy)methyl)imidazo[1,2-a]pyridin-8-yl)-3-methylimidazolidine-2,4-dione (I-97)

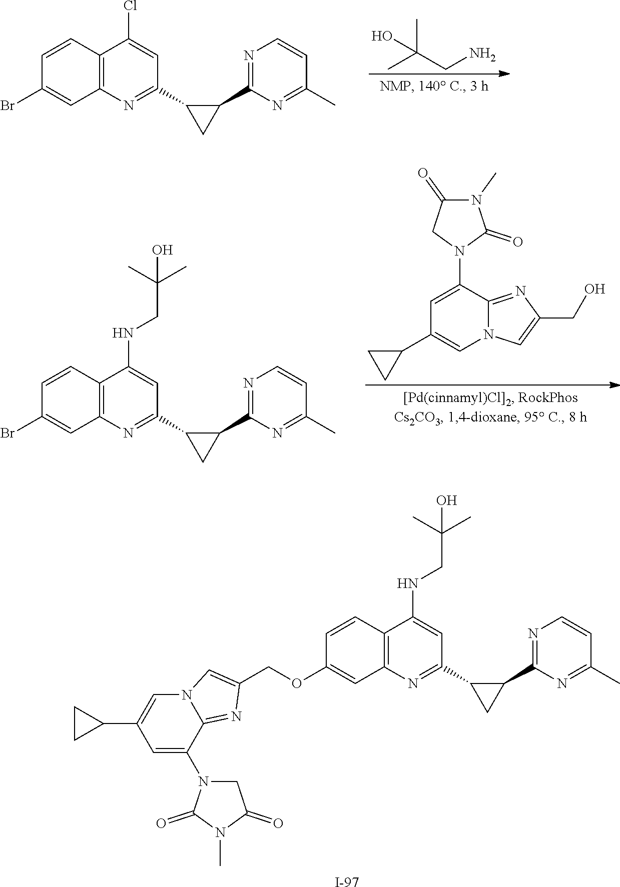

### Synthesis of 1-((7-bromo-2-((1S,2S)-2-(4-methylpyrimidin-2-yl)cyclopropyl)quinolin-4-yl)amino)-2-methylpropan-2-ol

A mixture of 7-bromo-4-chloro-2-((1S,2S)-2-(4-methylpyrimidin-2-yl)cyclopropyl)quinoline (0.30 g, 0.80 mmol) and 1-amino-2-methylpropan-2-ol (0.71 g, 8.0 mmol) in NMP (4.0 mL) was stirred in a sealed tube. After degassing with N₂ for 1 min, the mixture was irradiated in a microwave reactor at 140° C. for 3 h. After the reaction mixture was cooled to room temperature, water (25 mL) was added and then extracted with EtOAc (40 mL×3). The combined organic layers were washed with brine (40 mL), dried over anhydrous Na₂SO₄, and then concentrated in vacuo to give the crude, which was purified by preparative TLC, eluting with 0˜6% MeOH/DCM to give 1-((7-bromo-2-((1S,2S)-2-(4-methylpyrimidin-2-yl)cyclopropyl)quinolin-4-yl)amino)-2-methylpropan-2-ol (0.30 g, 88%) as a yellow solid.

ESI-MS [M+H]⁺: 427.2

### Synthesis of 1-(6-cyclopropyl-2-(((4-((2-hydroxy-2-methylpropyl)amino)-2-((1S,2S)-2-(4-methylpyrimidin-2-yl)cyclopropyl)quinolin-7-yl)oxy)methyl)imidazo[1,2-a]pyridin-8-yl)-3-methylimidazolidine-2,4-dione

A mixture of 1-((7-bromo-2-((1S,2S)-2-(4-methylpyrimidin-2-yl)cyclopropyl)quinolin-4-yl)amino)-2-methylpropan-2-ol (0.12 g, 0.28 mmol), 1-(6-cyclopropyl-2-(hydroxymethyl)imidazo[1,2-a]pyridin-8-yl)-3-methylimidazolidine-2,4-dione (0.21 g, 0.70 mmol), [Pd(cinnamyl)Cl]2 (22 mg, 0.042 mmol), RockPhos (39 mg, 0.084 mmol) and Cs₂CO₃ (0.27 g, 0.83 mmol) in 1,4-dioxane (20 mL) was stirred at 95° C. for 8 h under N₂. The reaction mixture was diluted with water (30 mL) and then extracted with EtOAc (50 mL×3). The combined organic layers were washed with brine (50 mL), dried over anhydrous Na₂SO₄, and then concentrated in vacuo to give a crude, which was purified by preparative TLC, eluting with 10% MeOH/DCM to give 1-(6-cyclopropyl-2-(((4-((2-hydroxy-2-methylpropyl)amino)-2-((1S,2S)-2-(4-methylpyrimidin-2-yl)cyclopropyl)quinolin-7-yl)oxy)methyl) imidazo[1,2-a]pyridin-8-yl)-3-methylimidazolidine-2,4-dione (30 mg, 17%) as a white solid.

ESI-MS [M+H]⁺: 648.3. ¹H NMR (400 MHz, DMSO) δ 8.53 (d, J=5.0 Hz, 1H), 8.31 (s, 1H), 8.14 (s, 1H), 7.99 (s, 1H), 7.39 (s, 1H), 7.28 (s, 1H), 7.19 (d, J=4.9 Hz, 1H), 7.09 (s, 1H), 6.55 (s, 1H), 5.32 (s, 2H), 4.91 (s, 2H), 4.66 (s, 1H), 3.31-3.20 (m, 2H), 2.97 (s, 3H), 2.86-2.71 (m, 1H), 2.65-2.57 (m, 1H), 2.42 (s, 3H), 2.05-1.93 (m, 1H), 1.88-1.61 (m, 2H), 1.19 (s, 6H), 1.05-0.93 (m, 2H), 0.73-0.61 (m, 2H).

### Example 98

### Synthesis of 1-(6-cyclopropyl-2-((methyl(2-((1S,2S)-2-(4-methylpyrimidin-2-yl)cyclopropyl)-4-(2-oxa-6-azaspiro[3.3]heptan-6-yl)quinolin-7-yl)amino)methyl)imidazo[1,2-a]pyridin-8-yl)-3-methylimidazolidine-2,4-dione (I-98)

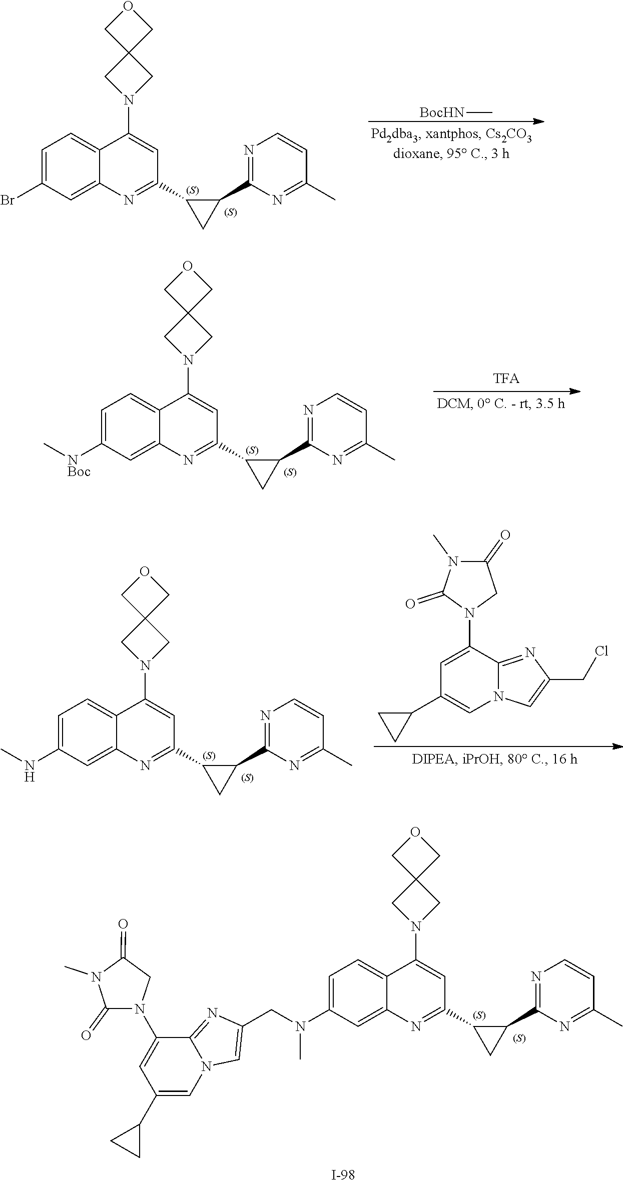

### Synthesis of tert-butyl methyl(2-((1S,2S)-2-(4-methylpyrimidin-2-yl)cyclopropyl)-4-(2-oxa-6-azaspiro[3.3]heptan-6-yl)quinolin-7-yl)carbamate

To a mixture of 6-(7-bromo-2-((1S,2S)-2-(4-methylpyrimidin-2-yl)cyclopropyl)quinolin-4-yl)-2-oxa-6-azaspiro[3.3]heptane (480 mg, 1.1 mmol) and tert-butyl methylcarbamate (183 mg, 1.4 mmol) in 1,4-dioxane (20 mL) was added Pd₂dba₃ (210 mg, 0.23 mmol), Xantphos (266 mg, 0.46 mmol) and Cs₂CO₃ (1.1 g, 3.4 mmol). The reaction mixture was stirred at 95° C. for 3 h under N₂. After the reaction was cooled to room temperature, water (30 mL) was added and the mixture was extracted with EtOAc (30 mL×3). The combined organic layers were washed with brine (30 mL), dried over Na₂SO₄, concentrated in vacuo to give the crude, Which was purification by column chromatography on silica gel (eluent: MeOH/DCM=1/20) to afford tert-butyl methyl(2-((1S,2S)-2-(4-methylpyrimidin-2-yl)cyclopropyl)-4-(2-oxa-6-azaspiro[3.3]heptan-6-yl)quinolin-7-yl)carbamate (400 mg, 74%) as a pale-yellow solid. ESI-MS [M+H]+: 488.2

### Synthesis of N-methyl-2-((1S,2S)-2-(4-methylpyrimidin-2-yl)cyclopropyl)-4-(2-oxa-6-azaspiro[3.3]heptan-6-yl)quinolin-7-amine

To a solution of tert-butyl methyl(2-((1S,2S)-2-(4-methylpyrimidin-2-yl)cyclopropyl)-4-(2-oxa-6-azaspiro[3.3]heptan-6-yl)quinolin-7-yl)carbamate (200 mg, 0.41 mmol) in DCM (2 mL) was added TFA (2 mL) at 0° C., the mixture was stirred at room temperature for 3.5 h. The reaction mixture was quenched with NaHCO₃ (sat. aq., 50 mL) and extracted with EtOAc (30 mL×3). The combined organic layers were washed with brine (30 mL), dried over Na₂SO₄ and concentrated in vacuo. The residue was purified by column chromatography (eluent: MeOH/DCM=0-5%) to afford N-methyl-2-((1S,2S)-2-(4-methylpyrimidin-2-yl)cyclopropyl)-4-(2-oxa-6-azaspiro[3.3]heptan-6-yl)quinolin-7-amine (142 mg, 89%) as a yellow solid. ESI-MS: [M+H]+, 388.2

### Synthesis of 1-(6-cyclopropyl-2-((methyl(2-((1S,2S)-2-(4-methylpyrimidin-2-yl)cyclopropyl)-4-(2-oxa-6-azaspiro[3.3]heptan-6-yl)quinolin-7-yl)amino)methyl)imidazo[1,2-a]pyridin-8-yl)-3-methylimidazolidine-2,4-dione

A mixture of N-methyl-2-((1S,2S)-2-(4-methylpyrimidin-2-yl)cyclopropyl)-4-(2-oxa-6-azaspiro[3.3]heptan-6-yl)quinolin-7-amine (80 mg, 0.21 mmol), 1-(2-(chloromethyl)-6-cyclopropylimidazo[1,2-a]pyridin-8-yl)-3-methylimidazolidine-2,4-dione (99 mg, 0.31 mmol) and DIPEA (130 mg, 1 mmol) in i-PrOH (4 mL) was stirred at 80° C. for 16 h. Water (30 mL) was added and the mixture was extracted with EtOAc (20 mL×3). The combined organic layers were washed with brine (20 mL), dried over Na₂SO₄ and concentrated in vacuo. The residue was purified by preparative TLC (eluent: DCM/MeOH=15/1) to afford 1-(6-cyclopropyl-2-((methyl(2-((1S,2S)-2-(4-methylpyrimidin-2-yl)cyclopropyl)-4-(2-oxa-6-azaspiro [3.3]heptan-6-yl)quinolin-7-yl)amino)methyl)imidazo[1,2-a]pyridin-8-yl)-3-methylimidazolidine-2,4-dione (40 mg, 29%) as a white solid.

ESI-MS [M+H]+: 670.3. ¹H NMR (400 MHz, DMSO) δ 8.53 (d, J=5.1 Hz, 1H), 8.19 (s, 1H), 7.74-7.67 (m, 2H), 7.35 (s, 1H), 7.20-7.12 (m, 2H), 6.90 (s, 1H), 5.99 (s, 1H), 4.86 (s, 2H), 4.77-4.74 (m, 6H), 4.57 (s, 4H), 3.17 (s, 3H), 2.96 (s, 3H), 2.77-2.74 (m, 1H), 2.64-2.61 (m, 1H), 2.42 (s, 3H), 1.93-1.89 (m, 1H), 1.83-1.81 (m, 1H), 1.72-1.70 (m, 1H), 0.93-0.89 (m, 2H), 0.63-0.60 (m, 2H).

### Example 99

### Synthesis of 1-(6-cyclopropyl-2-(1-((2-((1S,2S)-2-(4-methylpyrimidin-2-yl)cyclopropyl)-4-(2-oxa-6-azaspiro[3.3]heptan-6-yl)quinolin-7-yl)amino)ethyl)imidazo[1,2-a]pyridin-8-yl)-3-methylimidazolidine-2,4-dione (I-99)

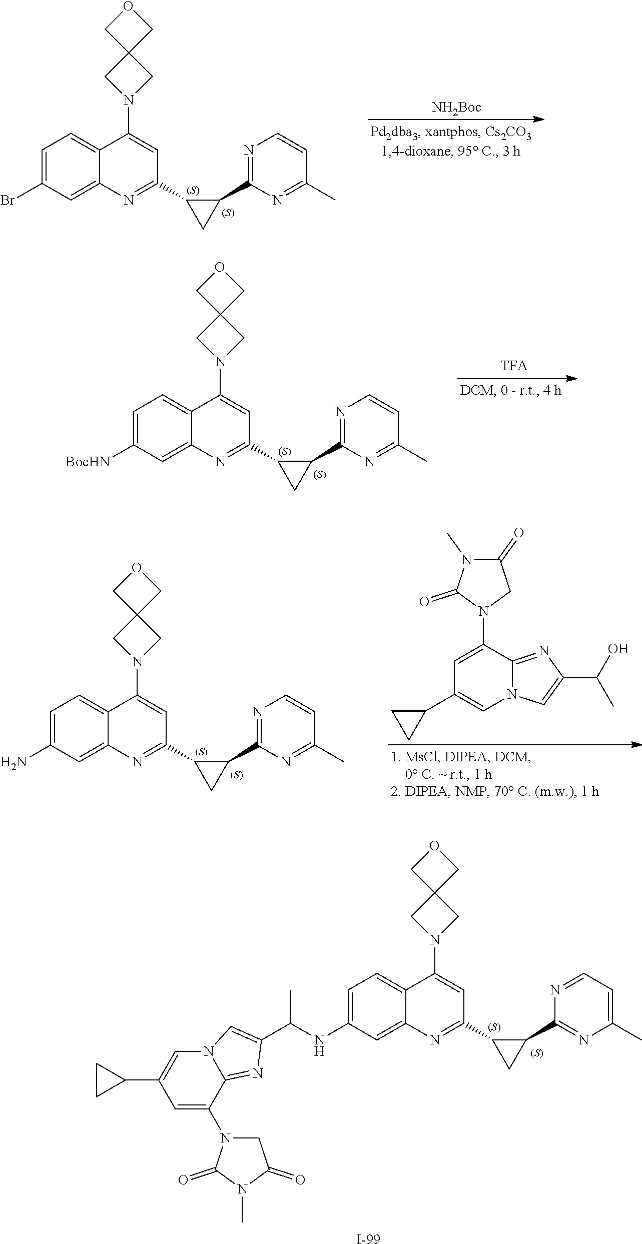

### Synthesis of tert-butyl (2-((1S,2S)-2-(4-methylpyrimidin-2-yl)cyclopropyl)-4-(2-oxa-6-azaspiro[3.3]heptan-6-yl)quinolin-7-yl)carbamate

To a mixture of 6-(7-bromo-2-((1S,2S)-2-(4-methylpyrimidin-2-yl)cyclopropyl)quinolin-4-yl)-2-oxa-6-azaspiro[3.3]heptane (500 mg, 1.1 mmol) and tert-butyl carbamate (164 mg, 1.4 mmol) in 1,4-dioxane (25 mL) was added Pd₂(dba)₃ (203 mg, 0.22 mmol), Xantphos (254 mg, 0.44 mmol) and Cs₂CO₃ (1.1 g, 3.3 mmol). The reaction mixture was stirred at 95° C. for 3 h. The mixture was cooled to room temperature, filtered and washed with DCM/MeOH (v/v=3/1, 50 mL). The filtrate was concentrated in vacuo and the residue was purified by column chromatography on silica gel (eluent: MeOH/DCM=0-5%) to afford tert-butyl (2-((1S,2S)-2-(4-methylpyrimidin-2-yl)cyclopropyl)-4-(2-oxa-6-azaspiro[3.3]heptan-6-yl)quinolin-7-yl)carbamate (400 mg, yield 77%) as a pale yellow solid.

ESI-MS [M+H]+: 474.2

### Synthesis of 2-((1S,2S)-2-(4-methylpyrimidin-2-yl)cyclopropyl)-4-(2-oxa-6-azaspiro[3.3]heptan-6-yl)quinolin-7-amine

To a solution of tert-butyl (2-((1S,2S)-2-(4-methylpyrimidin-2-yl)cyclopropyl)-4-(2-oxa-6-azaspiro[3.3]heptan-6-yl)quinolin-7-yl)carbamate (400 mg, 0.84 mmol) in DCM (10 mL) was added TFA (1.5 mL) at 0° C. The mixture was stirred at room temperature for 4 h. The mixture was concentrated in vacuo and the residue was neutralized with NH₃ (10 mL, 7 M in MeOH) and concentrated in vacuo to give the crude, which was purified by column chromatography on silica gel (eluent: MeOH/DCM=0-10%) to afford 2-((1S,2S)-2-(4-methylpyrimidin-2-yl)cyclopropyl)-4-(2-oxa-6-azaspiro[3.3]heptan-6-yl)quinolin-7-amine (160 mg, yield 51%) as a pale-yellow solid.

ESI-MS [M+H]+: 374.2

### Synthesis of 1-(6-cyclopropyl-2-(1-((2-((1S,2S)-2-(4-methylpyrimidin-2-yl)cyclopropyl)-4-(2-oxa-6-azaspiro[3.3]heptan-6-yl)quinolin-7-yl)amino)ethyl)imidazo[1,2-a]pyridin-8-yl)-3-methylimidazolidine-2,4-dione

To a mixture of 1-(6-cyclopropyl-2-(1-hydroxyethyl)imidazo[1,2-a]pyridin-8-yl)-3-methylimidazolidine-2,4-dione (100 mg, 0.32 mmol) and DIPEA (62 mg, 0.48 mmol) in DCM (3 mL) was added MsCl (55 mg, 0.48 mmol) at 0° C. The reaction mixture was stirred at room temperature for 1 h. The reaction solution was concentrated in vacuo. The residue was dissolved in NMP (2 mL), and then 2-((1S,2S)-2-(4-methylpyrimidin-2-yl)cyclopropyl)-4-(2-oxa-6-azaspiro[3.3]heptan-6-yl)quinolin-7-amine (120 mg, 0.32 mmol) and DIPEA (210 mg, 1.6 mmol) were added. The mixture was irradiated in a microwave reactor at 70° C. for 1 h under N₂. The mixture was cooled to room temperature, Water (30 mL) was added and the mixture extracted with EtOAc (30 mL×3). The combined organic layers were washed with brine (30 mL), dried over Na₂SO₄, concentrated in vacuo. The crude was purified by Prep-HPLC (FA) to afford 1-(6-cyclopropyl-2-(1-((2-((1S,2S)-2-(4-methylpyrimidin-2-yl)cyclopropyl)-4-(2-oxa-6-azaspiro[3.3]heptan-6-yl)quinolin-7-yl)amino)ethyl)imidazo[1,2-a]pyridin-8-yl)-3-methylimidazolidine-2,4-dione (3.9 mg, yield 2%) as white solid.

ESI-MS [M+H]+: 670.3, ¹H NMR (400 MHz, DMSO) δ 8.49 (d, J=5.1 Hz, 1H), 8.21-8.20 (m, 1H), 7.65 (s, 1H), 7.60 (d, J=9.2 Hz, 1H), 7.35 (d, J=1.4 Hz, 1H), 7.16 (d, J=5.1 Hz, 1H), 6.89-6.86 (m, 1H), 6.63 (s, 1H), 6.59 (d, J=2.1 Hz, 1H), 5.97 (s, 1H), 5.02-4.96 (m, 2H), 4.78-4.68 (m, 5H), 4.44 (s, 4H), 2.97 (s, 3H), 2.65-2.60 (m, 1H), 2.56-2.52 (m, 1H), 2.39 (s, 3H), 1.94-1.88 (m, 1H), 1.72-1.68 (m, 1H), 1.64-1.60 (m, 1H), 1.57 (d, J=6.7 Hz, 3H), 0.95-0.89 (m, 2H), 0.66-0.59 (m, 2H).

### Example 100

### Synthesis of 1-(6-cyclopropyl-2-(((4-((2-hydroxy-2-methylpropyl)(methyl)amino)-2-((1S,2S)-2-(4-methylpyrimidin-2-yl)cyclopropyl)quinolin-7-yl)amino)methyl)imidazo[1,2-a]pyridin-8-yl)-3-methylimidazolidine-2,4-dione (I-100)

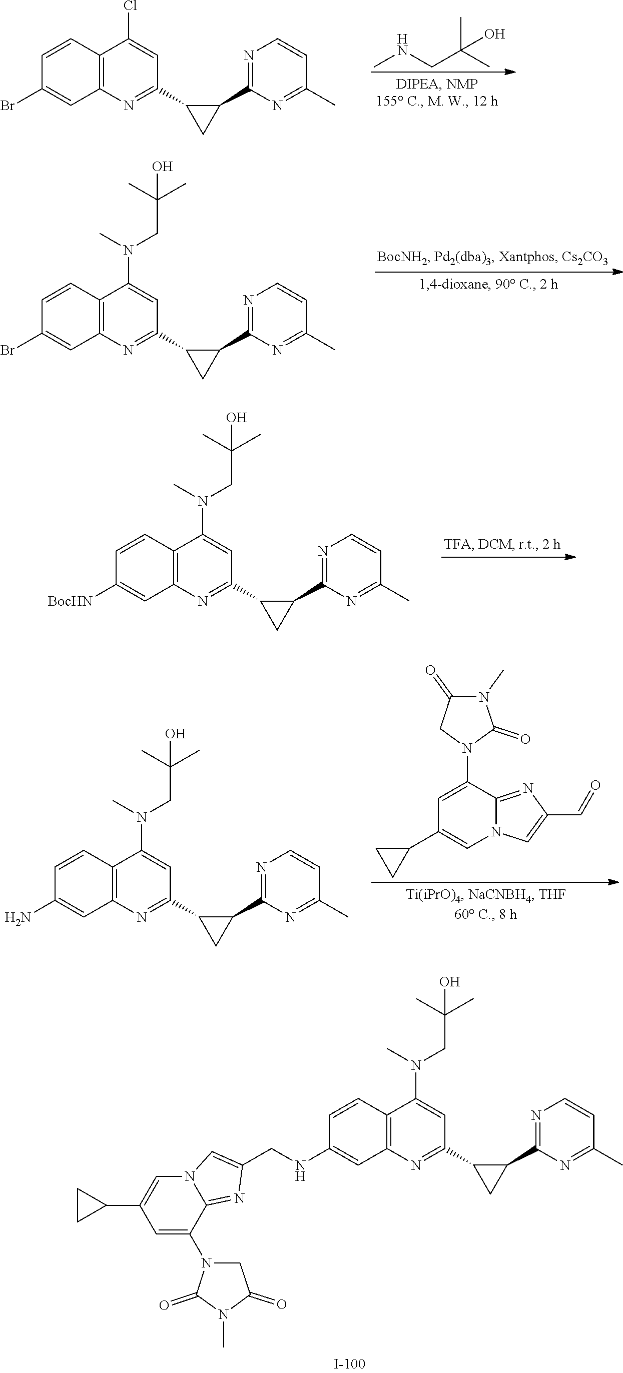

### Synthesis of 1-((7-bromo-2-((1S,2S)-2-(4-methylpyrimidin-2-yl)cyclopropyl)quinolin-4-yl)(methyl)amino)-2-methylpropan-2-ol

A mixture of 7-bromo-4-chloro-2-((1S,2S)-2-(4-methylpyrimidin-2-yl)cyclopropyl)quinoline (0.45 g, 1.2 mmol), DIPEA (0.76 g, 5.9 mmol) and 2-methyl-1-(methylamino)propan-2-ol (0.48 g, 0.47 mmol) in NMP (5 mL) was stirred in a sealed tube. After degassing with N₂ for 1 min, the reaction mixture was irradiated in a microwave reactor at 155° C. for 12 h. The resulting mixture was quenched with water (30 mL) and then extracted with EtOAc (50 mL×3). The combined organic layers were washed with brine, dried over Na₂SO₄, filtered and then concentrated 1-(6-cyclopropyl-2-(1-hydroxyethyl)imidazo[1,2-a]pyridin-8-yl)-3-methylimidazolidine-2,4-dione to give a crude, which was purified by preparative TLC, eluting with 4% MeOH/DCM to give 1-((7-bromo-2-((1S,2S)-2-(4-methylpyrimidin-2-yl) cyclopropyl)quinolin-4-yl)(methyl)amino)-2-methylpropan-2-ol (0.24 g, 46%) as a yellow solid. ESI-MS [M+H]⁺: 441.1.

### Synthesis of tert-butyl (4-((2-hydroxy-2-methylpropyl)(methyl)amino)-2-((1S,2S)-2-(4-methylpyrimidin-2-yl)cyclopropyl)quinolin-7-yl)carbamate

To a mixture of 1-((7-bromo-2-((1S,2S)-2-(4-methylpyrimidin-2-yl)cyclopropyl)quinolin-4-yl)(methyl)amino)-2-methylpropan-2-ol (0.24 g, 0.54 mmol), BocNH₂ (94 mg, 0.80 mmol) and Cs₂CO₃ (0.52 g, 1.6 mmol) in 1,4-dioxane (10 mL) was added Pd₂(dba)₃ (49 mg, 0.054 mmol) and Xantphos (62 mg, 0.11 mmol). After the reaction was stirred at 90° C. for 2 h under N₂, the reaction mixture was filtered through Celite® and the filter cake was washed with DCM/MeOH (V/V=10/1, 100 mL). The filtrate was concentrated in vacuo to give a crude, which was purified by preparative TLC, eluting with 6% MeOH/DCM to give tert-butyl (4-((2-hydroxy-2-methylpropyl)(methyl)amino)-2-((1S,2S)-2-(4-methylpyrimidin-2-yl)cyclopropyl)quinolin-7-yl)carbamate (0.17 g, 66%) as a yellow solid. ESI-MS [M+H]⁺: 478.3.

### Synthesis of 1-((7-amino-2-((1S,2S)-2-(4-methylpyrimidin-2-yl)cyclopropyl)quinolin-4-yl)(methyl)amino)-2-methylpropan-2-ol

To a solution of tert-butyl (4-((2-hydroxy-2-methylpropyl)(methyl)amino)-2-((1S,2S)-2-(4-methylpyrimidin-2-yl)cyclopropyl)quinolin-7-yl)carbamate (0.17 g, 0.36 mmol) in DCM (5 mL) was added TFA (1 mL) slowly at 0° C. The reaction was stirred at room temperature for 2 h, then concentrated in vacuo. The residue was diluted and neutralized with NH₃ (7 N NH₃ in MeOH). The resulting mixture was concentrated in vacuo and purified by preparative TLC, eluting with 10% MeOH/DCM to give 1-((7-amino-2-((1S,2S)-2-(4-methylpyrimidin-2-yl)cyclopropyl)quinolin-4-yl)(methyl)amino)-2-methylpropan-2-ol (0.10 g, 74%) as a yellow solid. ESI-MS [M+H]+: 378.2.

### Synthesis of 1-(6-cyclopropyl-2-(((4-((2-hydroxy-2-methylpropyl)(methyl)amino)-2-((1S,2S)-2-(4-methylpyrimidin-2-yl)cyclopropyl)quinolin-7-yl)amino)methyl)imidazo[1,2-a]pyridin-8-yl)-3-methylimidazolidine-2,4-dione

To a mixture of 1-((7-amino-2-((1S,2S)-2-(4-methylpyrimidin-2-yl)cyclopropyl)quinolin-4-yl)(methyl)amino)-2-methylpropan-2-ol (0.10 g, 0.27 mmol) and 6-cyclopropyl-8-(3-methyl-2,4-dioxoimidazolidin-1-yl)imidazo[1,2-a]pyridine-2-carbaldehyde (87 mg, 0.29 mmol) in THF (10 mL) was added Ti(iPrO)₄ (0.41 g, 1.4 mmol), the resulting mixture was stirred at 60° C. for 12 h, then cooled to room temperature. To the mixture was added MeOH (2 mL) and NaCNBH₃ (55 mg, 0.87 mmol), the resulting mixture was stirred at room temperature for another 2 h. Water (20 mL) was added and the reaction was extracted with EtOAc (20 mL×3). The combined organic layers were washed with brine, dried over Na₂SO₄, filtered and then concentrated in vacuo to give a crude, which was purified by Pre-TLC, eluting with 6% MeOH/DCM to give 1-(6-cyclopropyl-2-(((4-((2-hydroxy-2-methylpropyl)(methyl) amino)-2-((1S,2S)-2-(4-methylpyrimidin-2-yl)cyclopropyl)quinolin-7-yl)amino)methyl)imidazo[1,2-a]pyridin-8-yl)-3-methylimidazolidine-2,4-dione (25 mg, 14%) as a gray solid.

ESI-MS [M+H]+: 660.3. ¹H NMR (400 MHz, DMSO) δ 8.51 (d, J=5.1 Hz, 1H), 8.25 (s, 1H), 7.83 (d, J=9.3 Hz, 1H), 7.75 (s, 1H), 7.35 (d, J=1.3 Hz, 1H), 7.17 (d, J=5.1 Hz, 1H), 6.96-6.93 (m, 1H), 6.73 (s, 1H), 6.69 (d, J=2.1 Hz, 1H), 4.94 (s, 2H), 4.47-4.46 (m, 3H), 3.13 (s, 2H), 2.99 (s, 3H), 2.61-2.57 (m, 2H), 2.50 (s, 3H), 2.40 (s, 3H), 1.97-1.88 (m, 1H), 1.80-1.62 (m, 2H), 1.09 (s, 6H), 0.97-0.88 (m, 2H), 0.69-0.59 (m, 2H).

### Example 101

### Synthesis of 1-(6-cyclopropyl-2-((R)-1-((2-((1S,2S)-2-(4-methylpyrimidin-2-yl)cyclopropyl)-4-(2-oxa-6-azaspiro[3.3]heptan-6-yl)quinolin-7-yl)amino)ethyl)imidazo[1,2-a]pyridin-8-yl)-3-methylimidazolidine-2,4-dione (I-101)

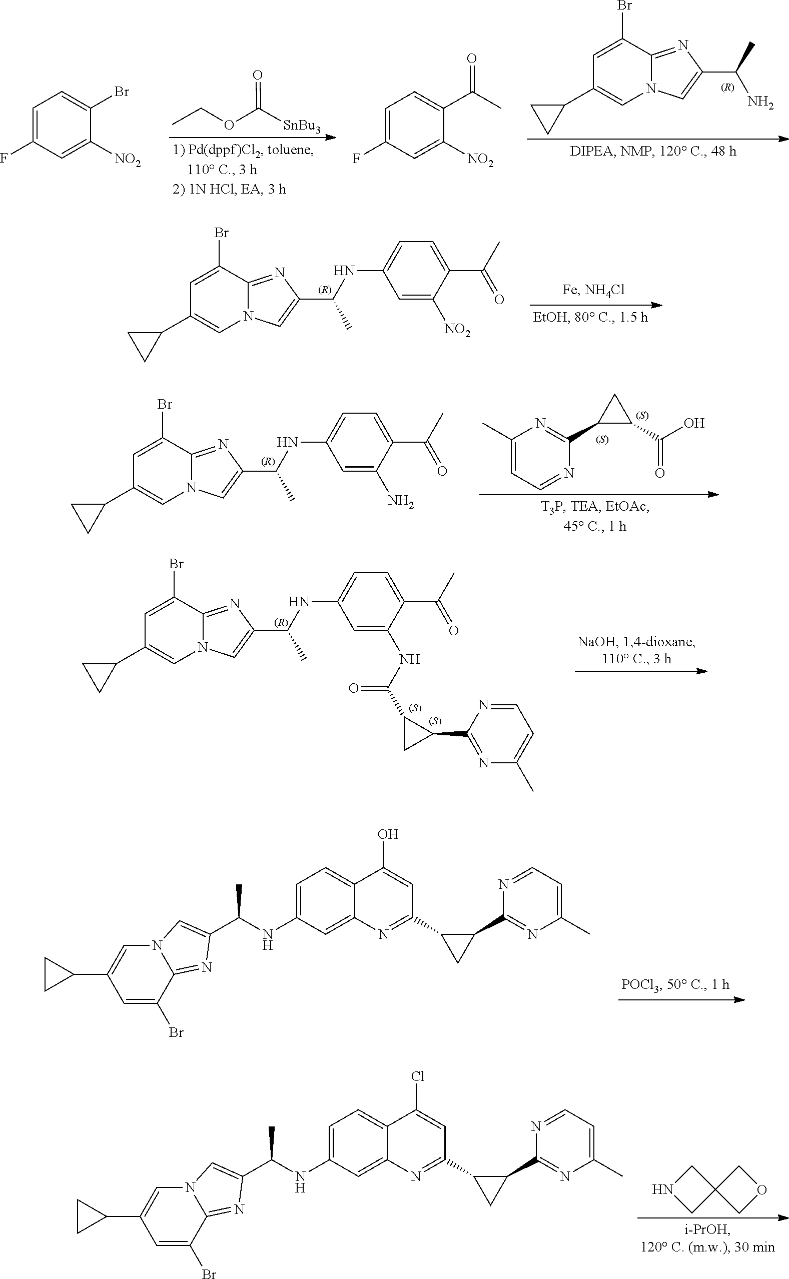

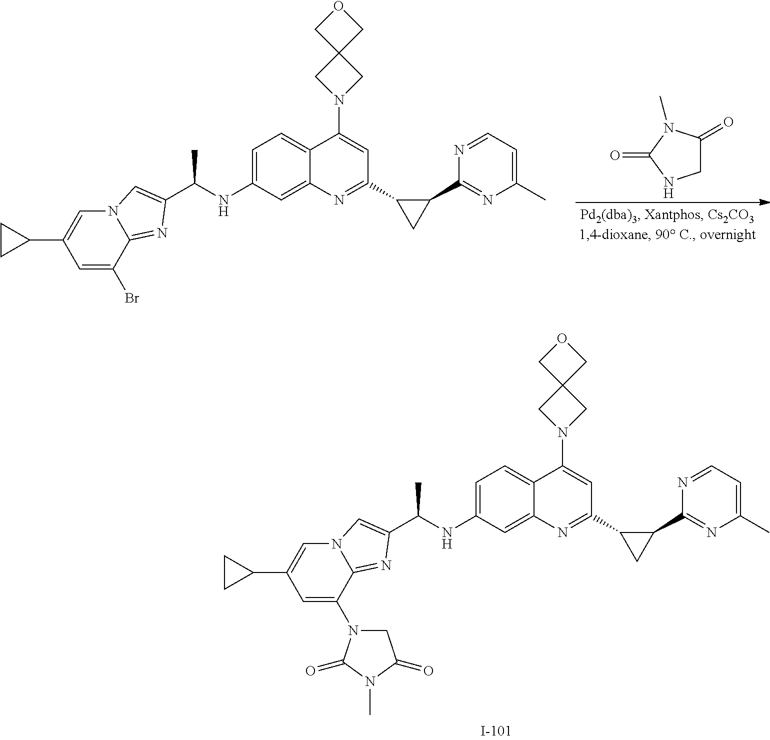

### Synthesis of (R)-1-(8-bromo-6-cyclopropylimidazo[1,2-a]pyridin-2-yl)ethan-1-amine hydrochloride

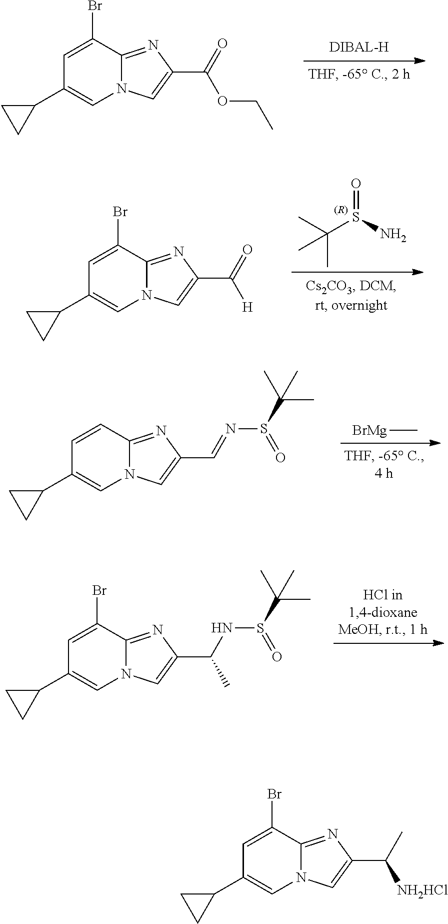

### Synthesis of 8-bromo-6-cyclopropylimidazo[1,2-a]pyridine-2-carbaldehyde

To a solution of ethyl 8-bromo-6-cyclopropylimidazo[1,2-a]pyridine-2-carboxylate (2 g, 6.5 mmol) in THF (40 mL) was added DIBAL-H (1.95 mL, 19.5 mmol) at −65° C. Stirring was continued for 2 h at the same temperature. The reaction mixture was quenched by adding sodium sulfate decahydrate at −65° C., warmed to 0° C. and stirred for 10 min. The mixture was filtered and the filtrate concentrated to give a residue, which was purified by silica gel chromatography (eluent: PE:EtOAc=5:1) to give 8-bromo-6-cyclopropylimidazo[1,2-a]pyridine-2-carbaldehyde (1.1 g, yield 64%). ESI-MS [M+H]+: 265.0

### Synthesis of (R,E)-N-((6-cyclopropylimidazo[1,2-a]pyridin-2-yl)methylene)-2-methylpropane-2-sulfinamide

To a solution of 8-bromo-6-cyclopropylimidazo[1,2-a]pyridine-2-carbaldehyde (4.2 g, 15.8 mmol) in DCM (80 mL) was added (R)-2-methylpropane-2-sulfinamide (2.5 g, 0.21 mmol) and Cs₂CO₃ (10.33 g, 0.21 mmol). The reaction mixture was stirred at room temperature overnight, then poured into water (100 mL) and extracted with EtOAc (100 mL×3). The combined organics were washed with brine (100 mL), dried over Na₂SO₄. The reaction mixture was concentrated in vacuo and purified by silica gel chromatography (eluent: PE:EtOAc=5:1) to give (R,E)-N-((8-bromo-6-cyclopropylimidazo[1,2-a]pyridin-2-yl)methylene)-2-methylpropane-2-sulfinamide (4.2 g, yield 72%). ESI-MS [M+H]+: 368.0

### Synthesis of (R)—N—((R)-1-(8-bromo-6-cyclopropylimidazo[1,2-a]pyridin-2-yl)ethyl)-2-methylpropane-2-sulfinamide

To a solution of (R,E)-N-((6-cyclopropylimidazo[1,2-a]pyridin-2-yl)methylene)-2-methylpropane-2-sulfinamide (200 mg, 0.54 mmol) in THF (5 mL) was added methylmagnesium bromide (0.72 mL, 2.16 mmol) at −65° C. and the mixture was stirred for 4 h at the same temperature. The reaction mixture was quenched with NH₄Cl solution (sat. aq., 50 mL) at −65° C., stirred for 10 min then extracted with EtOAc (50 mL×3). The combined organics were washed with brine (50 mL), dried over Na₂SO₄ and concentrated in vacuo. The residue was purified by silica gel chromatography (eluent: PE:EtOAc=5:1) to give (R)—N—((R)-1-(8-bromo-6-cyclopropylimidazo[1,2-a]pyridin-2-yl)ethyl)-2-methylpropane-2-sulfinamide (160 mg, yield 77%). ESI-MS [M+H]+: 384.1

### Synthesis of (R)-1-(8-bromo-6-cyclopropylimidazo[1,2-a]pyridin-2-yl)ethan-1-amine hydrochloride

To a solution of N—((R)-1-(8-bromo-6-cyclopropylimidazo[1,2-a]pyridin-2-yl)ethyl)-2-methylpropane-2-sulfinamide (1.5 g, 3.9 mmol) in MeOH (10 mL) was added HCl (4M solution in 1,4-dioxane, 10 mL). The resulting mixture was stirred at room temperature for 1 h and concentrated in vacuo to give (R)-1-(8-bromo-6-cyclopropylimidazo[1,2-a]pyridin-2-yl)ethan-1-amine hydrochloride (1.3 g, 94%) as a yellow solid. ESI-MS: [M+H]⁺, 280.0

### Synthesis of 1-(4-fluoro-2-nitrophenyl)ethan-1-one

To a mixture of 1-bromo-4-fluoro-2-nitrobenzene (13.0 g, 59.0 mmol) and ethyl tributylstannanecarboxylate (25 g, 69 mmol) in toluene (500 mL) was added Pd(dppf)₂Cl₂ (4.3 g, 5.9 mmol) and the reaction mixture was stirred at 110° C. for 3 h. The mixture was cooled to room temperature, filtered by Celite® and washed with EtOAc (200 mL). The filtrate was concentrated in vacuo. The residue was diluted with EtOAc (300 mL) and was added HCl (1 N, 50 mL). After the reaction was stirred for 3 h, the organic layer was separated, washed with brine (100 mL), dried over Na₂SO₄, filtered and concentrated in vacuo. The residue was purified by column chromatography on silica gel (eluent: EtOAc/PE=0-10%) to afford 1-(4-fluoro-2-nitrophenyl)ethan-1-one (7.6 g, yield 70%) as pale-yellow sticky oil. ESI-MS [M+H]+: 184.1

### Synthesis of (R)-1-(4-((1-(8-bromo-6-cyclopropylimidazo[1,2-a]pyridin-2-yl)ethyl)amino)-2-nitrophenyl)ethan-1-one

To a solution of (R)-1-(8-bromo-6-cyclopropylimidazo[1,2-a]pyridin-2-yl)ethan-1-amine hydrochloride (2.0 g, 6.3 mmol) in NMP (25 mL) was added 1-(4-fluoro-2-nitrophenyl)ethan-1-one (1.6 g, 8.9 mmol) and DIPEA (6.6 g, 51 mmol). The reaction mixture was stirred at 120° C. for 48 h. The mixture was cooled to room temperature, water (100 mL) was added and extracted with EtOAc (100 mL×3). The combined organic layers were washed with brine (100 mL), dried over Na₂SO₄, filtered, and concentrated in vacuo. The residue was purified by column chromatography on silica gel (eluent: EtOAc/PE=0-50%) to afford (R)-1-(4-((1-(8-bromo-6-cyclopropylimidazo[1,2-a]pyridin-2-yl)ethyl)amino)-2-nitrophenyl)ethan-1-one (1.7 g, yield 61%) as a pale-yellow solid. ESI-MS [M+H]+: 443.1

### Synthesis of (R)-1-(2-amino-4-((1-(8-bromo-6-cyclopropylimidazo[1,2-a]pyridin-2-yl)ethyl)amino)phenyl)ethan-1-one

To a mixture of (R)-1-(4-((1-(8-bromo-6-cyclopropylimidazo[1,2-a]pyridin-2-yl)ethyl)amino)-2-nitrophenyl)ethan-1-one (1.7 g, 3.8 mmol) and iron power (851 mg, 15.2 mmol) in EtOH (50 mL) was added a solution of NH₄Cl (806 g, 15.2 mmol) in H₂O (3.0 mL) and the resulting mixture was stirred at 80° C. for 1.5 h. The mixture was cooled to room temperature, filtered through Celite® and the filter cake was washed with EtOAc (50 mL). The filtrate was concentrated in vacuo. The residue was purified by column chromatography on silica gel (eluent: EtOAc/PE=0-50%) to afford (R)-1-(2-amino-4-((1-(8-bromo-6-cyclopropylimidazo[1,2-a]pyridin-2-yl)ethyl)amino)phenyl) ethan-1-one (1.1 g, yield 69%) as pale-yellow solid. ESI-MS [M+H]+: 413.1

### Synthesis of (1S,2S)—N-(2-acetyl-5-(((R)-1-(8-bromo-6-cyclopropylimidazo[1,2-a]pyridin-2-yl)ethyl)amino)phenyl)-2-(4-methylpyrimidin-2-yl)cyclopropane-1-carboxamide

To a solution of (R)-1-(2-amino-4-((1-(8-bromo-6-cyclopropylimidazo[1,2-a]pyridin-2-yl)ethyl)amino)phenyl)ethan-1-one (990 mg, 2.4 mmol), (1S,2S)-2-(4-methylpyrimidin-2-yl)cyclopropane-1-carboxylic acid (428 mg, 2.4 mmol) and TEA (1.2 g, 12 mmol) in EtOAc (20 mL) was added T3P (7.6 g, 12 mmol, 50% solution in EtOAc). The mixture was stirred at 45° C. for 1 h and cooled to room temperature. Water (50 mL) was added and extracted with EtOAc (50 mL×3). The combined organic layers were washed with brine (40 mL), dried over Na₂SO₄ and concentrated in vacuo. The residue was purified by column chromatography on silica gel (eluent: MeOH/DCM=0-10%) to afford (1S,2S)—N-(2-acetyl-5-(((R)-1-(8-bromo-6-cyclopropylimidazo[1,2-a]pyridin-2-yl)ethyl)amino)phenyl)-2-(4-methylpyrimidin-2-yl)cyclopropane-1-carboxamide (1.2 g, yield 86%) as a brown solid.

ESI-MS [M+H]+: 573.3

### Synthesis of 7-(((R)-1-(8-bromo-6-cyclopropylimidazo[1,2-a]pyridin-2-yl)ethyl)amino)-2-((1S,2S)-2-(4-methylpyrimidin-2-yl)cyclopropyl)quinolin-4-ol

To a solution of (1S,2S)—N-(2-acetyl-5-(((R)-1-(8-bromo-6-cyclopropylimidazo[1,2-a]pyridin-2-yl)ethyl)amino)phenyl)-2-(4-methylpyrimidin-2-yl)cyclopropane-1-carboxamide (1 g, 1.7 mmol) in 1,4-dioxane (50 mL) was added NaOH (280 mg, 7.0 mmol). The reaction mixture was stirred at 110° C. for 3 h. The mixture was cooled to room temperature, water (50 mL) was added and extracted with EtOAc (50 mL×3). The combined organic layers were washed with brine (40 mL), dried over anhydrous Na₂SO₄ and concentrated in vacuo. The residue was purified by column chromatography on silica gel (eluent: MeOH/DCM=0 10%) to afford 7-(((R)-1-(8-bromo-6-cyclopropylimidazo [1,2-a]pyridin-2-yl)ethyl)amino)-2-((1S,2S)-2-(4-methylpyrimidin-2-yl)cyclopropyl)quinolin-4-ol (650 mg, yield 69%) as a pale yellow solid. ESI-MS [M+H]+: 555.3

### Synthesis of N—((R)-1-(8-bromo-6-cyclopropylimidazo[1,2-a]pyridin-2-yl)ethyl)-4-chloro-2-((1S,2S)-2-(4-methylpyrimidin-2-yl)cyclopropyl)quinolin-7-amine

A solution of 7-(((R)-1-(8-bromo-6-cyclopropylimidazo [1,2-a]pyridin-2-yl)ethyl)amino)-2-((1S,2S)-2-(4-methylpyrimidin-2-yl)cyclopropyl)quinolin-4-ol (630 mg, 1.1 mmol) in POCl₃ (10 mL) was stirred at 50° C. for 1 h. The mixture was concentrated in vacuo and the residue was poured into ice water (50 mL) and neutralized with NaHCO₃ (sat. aq. 100 mL). Then the mixture was extracted with DCM (100 mL×3). The combined organic layers were washed with brine (50 mL), dried over Na₂SO₄, concentrated in vacuo. The residue was purified by column chromatography on silica gel (eluent: MeOH/DCM=0-10%) to afford N—((R)-1-(8-bromo-6-cyclopropylimidazo[1,2-a]pyridin-2-yl)ethyl)-4-chloro-2-((1S,2S)-2-(4-methylpyrimidin-2-yl)cyclo propyl)quinolin-7-amine (500 mg, yield 79%) as pale-yellow solid.

ESI-MS [M+H]+: 573.3

### Synthesis of N—((R)-1-(8-bromo-6-cyclopropylimidazo[1,2-a]pyridin-2-yl)ethyl)-2-((1S,2S)-2-(4-methylpyrimidin-2-yl)cyclopropyl)-4-(2-oxa-6-azaspiro[3.3]heptan-6-yl)quinolin-7-amine

A mixture of N—((R)-1-(8-bromo-6-cyclopropylimidazo[1,2-a]pyridin-2-yl)ethyl)-4-chloro-2-((1S,2S)-2-(4-methylpyrimidin-2-yl)cyclo propyl)quinolin-7-amine (250 mg, 0.44 mmol) and 2-oxa-6-azaspiro[3.3]heptane (218 mg, 2.2 mmol) in i-PrOH (8.0 mL) was irradiated in a microwave reactor at 120° C. for 0.5 h. The mixture was cooled to room temperature, water (50 mL) was added and the mixture extracted with EtOAc (50 mL×3). The combined organic layers were washed with brine (30 mL), dried over Na₂SO₄ and concentrated in vacuo to give the crude, which was purified by column chromatography on silica gel (eluent: MeOH/DCM=0-8%) to afford N—((R)-1-(8-bromo-6-cyclopropylimidazo[1,2-a]pyridin-2-yl)ethyl)-2-((1S,2S)-2-(4-methylpyrimidin-2-yl)cyclopropyl)-4-(2-oxa-6-azaspiro[3.3]heptan-6-yl)quinolin-7-amine (100 mg, yield 36%) as a pale-yellow solid. ESI-MS [M+H]+: 636.3

### Synthesis of 1-(6-cyclopropyl-2-((R)-1-((2-((1S,2S)-2-(4-methylpyrimidin-2-yl)cyclopropyl)-4-(2-oxa-6-azaspiro[3.3]heptan-6-yl)quinolin-7-yl)amino)ethyl)imidazo[1,2-a]pyridin-8-yl)-3-methylimidazolidine-2,4-dione

To a mixture of N—((R)-1-(8-bromo-6-cyclopropylimidazo[1,2-a]pyridin-2-yl)ethyl)-2-((1S,2S)-2-(4-methylpyrimidin-2-yl)cyclopropyl)-4-(2-oxa-6-azaspiro[3.3]heptan-6-yl)quinolin-7-amine (95 mg, 0.15 mmol) and 3-methylimidazolidine-2,4-dione (68 mg, 0.60 mmol) in 1,4-dioxane (5.0 mL) was added Pd₂(dba)₃ (41 mg, 0.045 mmol), Xantphos (52 mg, 0.090 mmol) and Cs₂CO₃ (147 mg, 0.45 mmol). The reaction mixture was stirred at 90° C. overnight under N₂. The mixture was filtered and washed with DCM/MeOH (v/v=3/1, 30 mL). The filtrate was concentrated in vacuo and the residue was purified by Prep-HPLC to afford 1-(6-cyclopropyl-2-((R)-1-((2-((1S,2S)-2-(4-methylpyrimidin-2-yl)cyclopropyl)-4-(2-oxa-6-azaspiro[3.3]heptan-6-yl)quinolin-7-yl)amino)ethyl)imidazo[1,2-a]pyridin-8-yl)-3-methylimidazolidine-2,4-dione (16 mg, 16%) as a white solid.

ESI-MS [M+H]+: 670.3. ¹H NMR (400 MHz, DMSO) δ 8.49 (d, J=5.1 Hz, 1H), 8.20 (d, J=1.1 Hz, 1H), 7.64 (s, 1H), 7.57 (d, J=9.1 Hz, 1H), 7.33 (d, J=1.5 Hz, 1H), 7.15 (d, J=5.1 Hz, 1H), 6.85 (dd, J=9.2, 2.3 Hz, 1H), 6.56 (d, J=2.3 Hz, 1H), 6.47 (s, 1H), 6.00 (s, 1H), 4.97 (d, J=7.1 Hz, 2H), 4.77-4.70 (m, 5H), 4.38 (s, 4H), 2.97 (s, 3H), 2.63-2.58 (m, 1H), 2.47-2.45 (m, 1H), 2.39 (s, 3H), 1.93-1.86 (m, 1H), 1.69-1.64 (m, 1H), 1.60-1.56 (m, 4H), 0.93-0.88 (m, 2H), 0.63-0.58 (m, 2H).

### Example 102

### Synthesis of rac-1-(6-cyclopropyl-2-(((5-methoxy-2-((1S*,2S*)-2-(4-methylpyrimidin-2-yl)cyclopropyl)quinolin-7-yl)oxy)methyl)imidazo[1,2-a]pyridin-8-yl)-3-methylimidazolidine-2,4-dione (I-102)

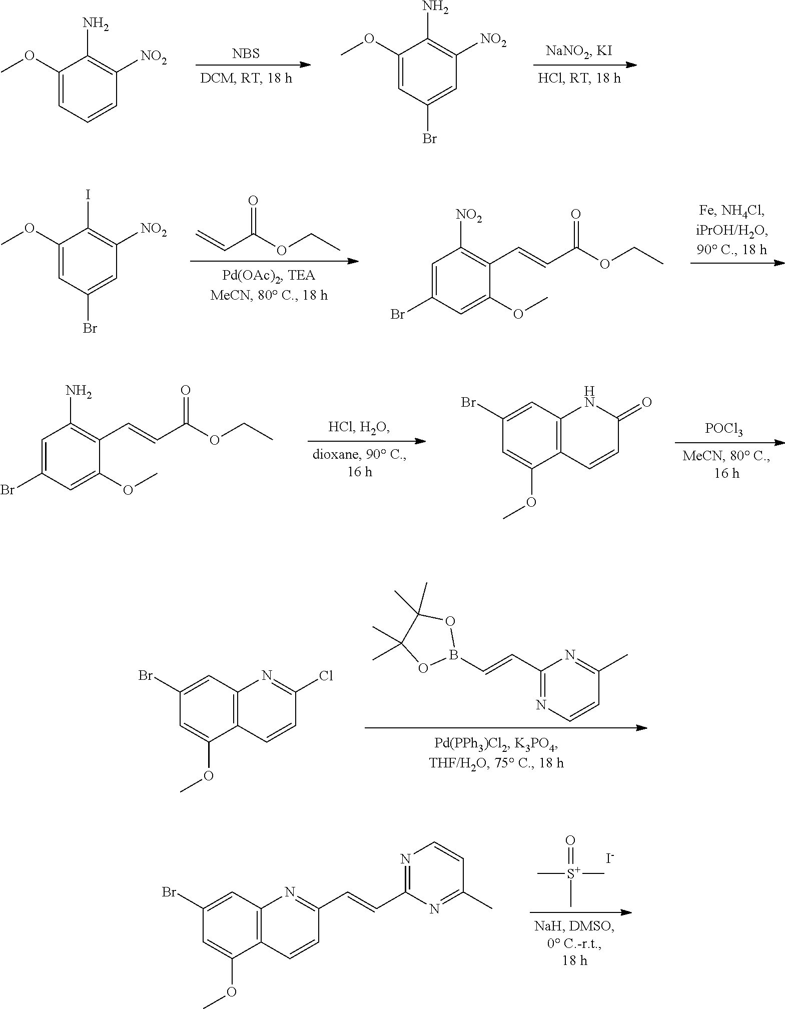

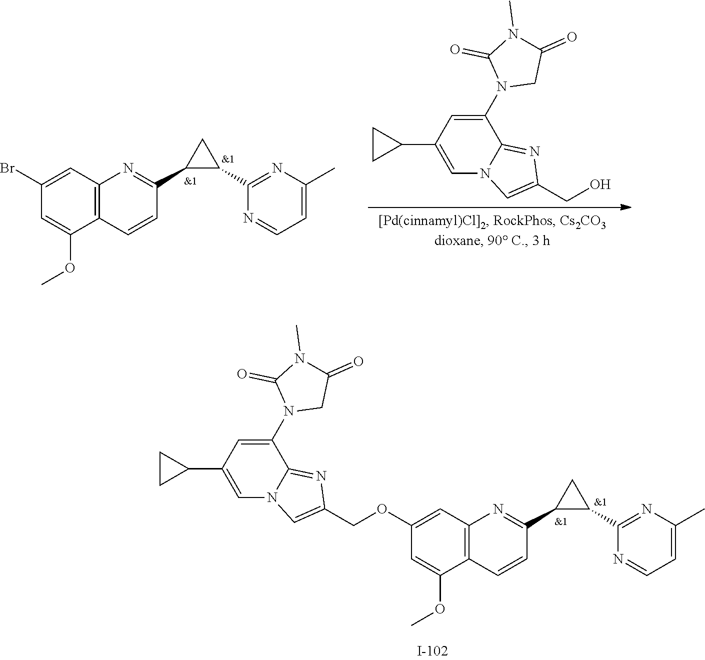

### Synthesis of 4-bromo-2-methoxy-6-nitroaniline

To a solution of 2-methoxy-6-nitroaniline (20 g, 119 mmol) in DCM (200 mL) was added NBS (21 g, 119 mmol) portionwise at 0° C. and the mixture was stirred at room temperature for 18 h. The mixture was quenched with Na₂SO₄ (sat. aq., 200 mL) and extracted with DCM (200 mL×3). The combined organic layers were washed with brine (200 mL), dried over Na₂SO₄ and concentrated in vacuo. The residue was purified by column chromatography (eluent: EtOAc/PE from 0 to 30%) to afford 4-bromo-2-methoxy-6-nitroaniline (21 g, 72% yield) as a red solid. ESI-MS [M+H]+: 247.0

### Synthesis of 5-bromo-2-iodo-1-methoxy-3-nitrobenzene

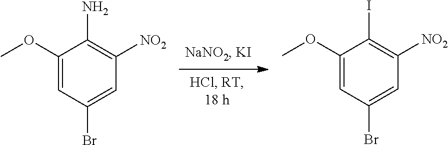

To a solution of 4-bromo-2-methoxy-6-nitroaniline (15 g, 61 mmol) in conc. HCl (150 mL) and ice-water (200 mL) was added a solution of NaNO₂ (6.3 g, 92 mmol) in H₂O (100 mL) at 0° C. and the mixture was stirred at room temperature for 1 h. KI (30 g, 183 mmol) in H₂O (200 mL) was added and the mixture was stirred at room temperature for 17 h. The mixture was then extracted with DCM (300 mL×3). The combined organic layers were washed with brine (300 mL), dried over Na₂SO₄ and concentrated in vacuo. The residue was purified by column chromatography (eluent: EtOAc/PE from 0 to 15%) to afford 5-bromo-2-iodo-1-methoxy-3-nitrobenzene (18 g, 83% yield) as a yellow solid. ESI-MS [M+H]+: 357.8

### Synthesis of ethyl (E)-3-(4-bromo-2-methoxy-6-nitrophenyl)acrylate

A mixture of afford 5-bromo-2-iodo-1-methoxy-3-nitrobenzene (5.0 g, 14.0 mmol), ethyl acrylate (1.54 g, 15.4 mmol), Pd(OAc)₂ (314 mg, 1.4 mmol) and TEA (4.24 g, 42.0 mmol) in MeCN (50 mL) in a sealed tube was stirred at 80° C. under N₂ for 18 h. The mixture was cooled to room temperature, water (100 mL) was added and the mixture extracted with EtOAc (50 mL×3). The combined organic layers were washed with brine (50 mL), dried over Na₂SO₄ and concentrated in vacuo. The residue was purified by column chromatography (eluent: EtOAc/PE from 0 to 15%) to afford ethyl (E)-3-(4-bromo-2-methoxy-6-nitrophenyl)acrylate (2.9 g, 63% yield) as a yellow solid.

ESI-MS [M+H]+: 330.0

### Synthesis of ethyl (E)-3-(2-amino-4-bromo-6-methoxyphenyl)acrylate

To a solution of ethyl (E)-3-(4-bromo-2-methoxy-6-nitrophenyl)acrylate (2.9 g, 8.8 mmol) in IPA (30 mL) and H₂O (15 mL) was added Fe (4.9 g, 88 mmol) and NH₄Cl (4.7 g, 56 mmol), the resulting mixture was stirred at 90° C. for 18 h. After the reaction was cooled to room temperature, the mixture was filtered, and the filtrate was extracted with DCM (50 mL×3). The combined organic layers were washed with brine (50 mL), dried over Na₂SO₄ and concentrated in vacuo. The residue was purified by flash column chromatography (eluent: EtOAc/PE from 0 to 15%) to afford the title compound (2.0 g, 76%) as a yellow solid.

ESI-MS [M+H]+: 300.0

### Synthesis of 7-bromo-5-methoxyquinolin-2 (1H)-one

Ethyl (E)-3-(2-amino-4-bromo-6-methoxyphenyl)acrylate (2.0 g, 6.7 mmol) in 1,4-dioxane (15 mL) and conc. HCl (20 mL) was stirred at 90° C. for 16 h. The mixture was cooled to room temperature and concentrated in vacuo, the residue was diluted with NaHCO₃ (sat. aq., 50 mL) and extracted with EtOAc (50 mL×3). The combined organic layers were washed with brine (30 mL), dried over Na₂SO₄ and concentrated in vacuo. The residue was purified by column chromatography (eluent: EtOAc/PE from 0 to 100%) to afford 7-bromo-5-methoxyquinolin-2 (1H)-one (1.5 g, 88%) as an off-white solid. ESI-MS [M+H]+: 254.0

### Synthesis of 7-bromo-2-chloro-5-methoxyquinoline

A mixture of 7-bromo-5-methoxyquinolin-2 (1H)-one (1.5 g, 5.9 mmol) and POCl3 (4.5 g, 29.5 mmol) in MeCN (20 mL) was stirred at 80° C. for 16 h. The mixture was cooled to room temperature and concentrated in vacuo, the residue was diluted with NaHCO₃ (sat. aq., 50 mL) and extracted with EtOAc (50 mL×3). The combined organic layers were washed with brine (30 mL), dried over Na₂SO₄ and concentrated in vacuo. The residue was purified by column chromatography (eluent: EtOAc/PE from 0 to 50%) to afford 7-bromo-2-chloro-5-methoxyquinoline (1.4 g, 88%) as an off-white solid. ESI-MS [M+H]+: 271.9

### Synthesis of (E)-7-bromo-5-methoxy-2-(2-(4-methylpyrimidin-2-yl)vinyl)quinoline

A mixture of 7-bromo-2-chloro-5-methoxyquinoline (1.3 g, 4.8 mmol), (E)-4-methyl-2-(2-(4,4,5,5-tetramethyl-1,3,2-dioxaborolan-2-yl)vinyl)pyrimidine (1.2 g, 4.8 mmol), Pd(PPh₃)₂Cl₂ (366 mg, 0.5 mmol) and K₃PO₄ (3.1 g, 14.4 mmol) in THF/H₂O (20 mL/2 mL) was stirred at 75° C. for 18 h under N₂. The mixture was cooled to room temperature, water (50 mL) was added and the mixture was extracted with EtOAc (50 mL×3). The combined organic layers were washed with brine (30 mL), dried over Na₂SO₄ and concentrated in vacuo. The residue was purified by flash column chromatography (eluent: EtOAc/PE from 0 to 40%) to afford E-7-bromo-5-methoxy-2-(2-(4-methylpyrimidin-2-yl)vinyl)quinoline (850 mg, 50%) as a white solid. ESI-MS [M+H]+: 356.0

### Synthesis of rac-7-bromo-5-methoxy-2-((1S*,2S*)-2-(4-methylpyrimidin-2-yl)cyclopropyl)quinoline

To a solution of trimethylsulfoxonium iodide (880 mg, 4.0 mmol) in DMSO (10 mL) was added NaH (160 mg, 60% in mineral oil, 4.0 mmol) at 0° C. and the mixture was stirred at room temperature for 1 h. E-7-bromo-5-methoxy-2-(2-(4-methylpyrimidin-2-yl)vinyl)quinoline (355 mg, 1.0 mmol) in DMSO (2 mL) was added and the mixture was stirred at room temperature for 17 h. The reaction was quenched with NH₄Cl (100 mL) and extracted with EtOAc (50 mL×3). The combined organic layers were washed with brine (30 mL), dried over Na₂SO₄ and concentrated in vacuo. The residue was purified by Prep-TLC (eluent: EtOAc/PE=1/2) to afford rac-7-bromo-5-methoxy-2-((1S*,2S*)-2-(4-methylpyrimidin-2-yl)cyclopropyl)quinoline (140 mg, 38%) as a white solid. ESI-MS [M+H]+: 370.0

### Synthesis of rac-1-(6-cyclopropyl-2-(((5-methoxy-2-((1S*,2S*)-2-(4-methylpyrimidin-2-yl)cyclopropyl)quinolin-7-yl)oxy)methyl)imidazo[1,2-a]pyridin-8-yl)-3-methylimidazolidine-2,4-dione

A mixture of rac-7-bromo-5-methoxy-2-((1S*,2S*)-2-(4-methylpyrimidin-2-yl)cyclopropyl)quinoline (80 mg, 0.22 mmol), 1-(6-cyclopropyl-2-(hydroxymethyl)imidazo[1,2-a]pyridin-8-yl)-3-methylimidazolidine-2,4-dione (66 mg, 0.22 mmol), [Pd(cinnamyl)Cl]₂ (23 mg, 0.044 mmol) RockPhos (41 mg, 0.088 mmol) and Cs₂CO₃ (215 mg, 0.66 mmol) in 1,4-dioxane (10 mL) was stirred at 90° C. for 3 h under N₂. The reaction mixture was filtered through Celite® and the filter cake was washed with DCM/MeOH (10/1, 100 mL). The filtrate was concentrated in vacuo to give the crude, which was purified by column chromatography Prep-TLC (eluent: MeOH/DCM=1/15) to afford rac-1-(6-cyclopropyl-2-(((5-methoxy-2-((1S*,2S*)-2-(4-methylpyrimidin-2-yl)cyclopropyl)quinolin-7-yl)oxy)methyl)imidazo[1,2-a]pyridin-8-yl)-3-methylimidazolidine-2,4-dione (15 mg, 12%) as an off-white solid.

ESI-MS [M+H]+: 590.3. ¹H NMR (400 MHz, DMSO) δ 8.53 (d, J=5.1 Hz, 1H), 8.32 (s, 1H), 8.25 (d, J=8.5 Hz, 1H), 8.02 (s, 1H), 7.40 (s, 1H), 7.35 (d, J=8.5 Hz, 1H), 7.19 (d, J=5.1 Hz, 1H), 7.07 (s, 1H), 6.66 (d, J=1.7 Hz, 1H), 5.33 (s, 2H), 4.93 (s, 2H), 3.94 (s, 3H), 2.98 (s, 3H), 2.82-2.74 (m, 2H), 2.42 (s, 3H), 2.00-1.93 (m, 1H), 1.85-1.74 (m, 2H), 1.02-0.92 (m, 2H), 0.73-0.64 (m, 2H).

### Example 103

### Synthesis of 1-(6-cyclopropyl-2-(((2-((1S,2S)-2-(4-methylpyrimidin-2-yl)cyclopropyl)-4-(2-oxa-6-azaspiro[3.3]heptan-6-yl)-1,6-naphthyridin-7-yl)oxy)methyl)imidazo[1,2-a]pyridin-8-yl)-3-methylimidazolidine-2,4-dione (I-103)

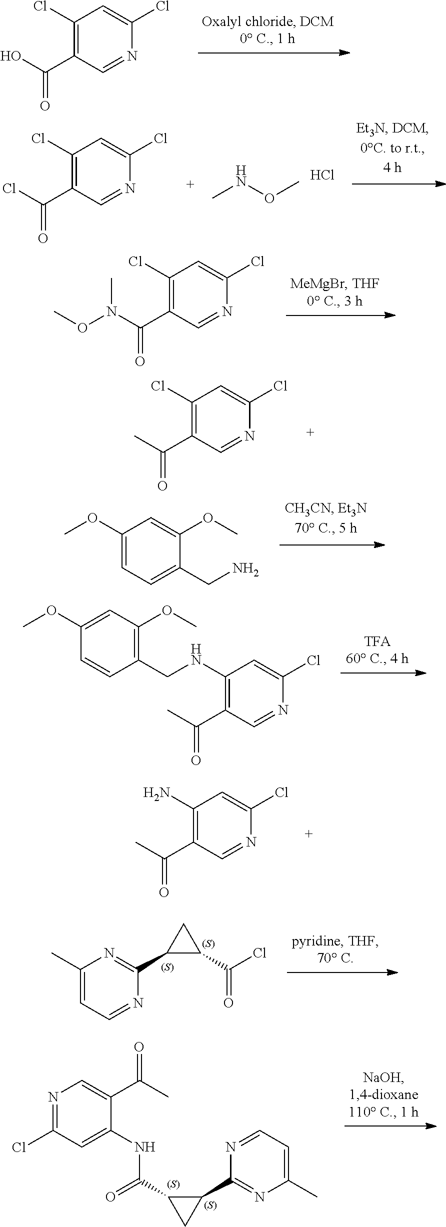

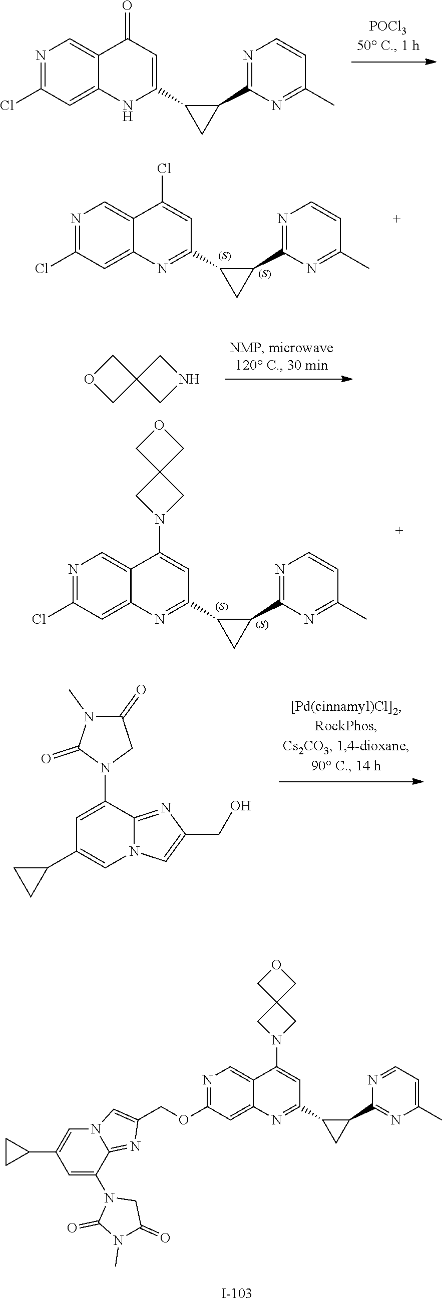

### Synthesis of 4,6-dichloronicotinoyl chloride

To a stirred solution of 4,6-dichloronicotinic acid (10 g, 52 mmol) in DCM (100 mL) was added SOCl₂ (9.9 g, 78 mmol) and DMF (0.10 mL) at 0° C. The mixture was stirred at room temperature for 1 h. The reaction mixture was concentrated and dried in vacuo to give 4,6-dichloronicotinoyl chloride (11 g, quant.) as a light brown syrup. ESI-MS [M+H]⁺: 206.0 (in MeOH).

### Synthesis of 4,6-dichloro-N-methoxy-N-methylnicotinamide

To a stirred solution of N,O-dimethylhydroxylamine hydrochloride (6.5 g, 67 mmol) and Et₃N (16 g, 0.16 mol) in DCM (100 mL) was added dropwise a solution of 4,6-dichloronicotinoyl chloride (11 g, 52 mmol) in DCM (50 mL) at 0° C. The mixture was stirred at room temperature for 4 h. The reaction mixture was washed with water (150 mL) and brine (150 mL), dried over Na₂SO₄, concentrated in vacuo to give the crude product, which was purified by column chromatography on silica gel, eluting with 0˜10% EtOAc in PE to give 4,6-dichloro-N-methoxy-N-methylnicotinamide (11 g, 90%) as a white solid. ESI-MS [M+H]⁺: 235.0.

### Synthesis of 1-(4,6-dichloropyridin-3-yl)ethan-1-one

To a stirred solution of 4,6-dichloro-N-methoxy-N-methylnicotinamide (11 g, 47 mmol) in THF (200 mL) was added dropwise MeMgBr (20 mL, 3 M in Et₂O, 60 mmol) at 0° C. The mixture was stirred at 0° C. for 3 h. The reaction mixture was quenched with NH₄Cl (sat. aq., 50 mL) and extracted with EtOAc (120 mL×2). The combined organics were washed with brine (200 mL), dried over anhydrous Na₂SO₄, concentrated and dried in vacuo to give 1-(4,6-dichloropyridin-3-yl)ethan-1-one (8.9 g, quant.) as a yellow solid. ESI-MS [M+H]⁺: 190.0.

### Synthesis of 1-(6-chloro-4-((2,4-dimethoxybenzyl)amino)pyridin-3-yl)ethan-1-one

A mixture of 1-(4,6-dichloropyridin-3-yl)ethan-1-one (8.9 g, 47 mmol), (2,4-dimethoxyphenyl)methanamine (7.8 g, 47 mmol) and Et₃N (14 g, 140 mol) in CH₃CN (90 mL) was stirred at 70° C. for 5 h. The reaction mixture was cooled to room temperature and concentrated in vacuo to give a crude. The residue was redissolved in DCM (100 mL) and washed with water (100 mL), organic layer was separated and concentrated in vacuo to give 1-(6-chloro-4-((2,4-dimethoxybenzyl)amino)pyridin-3-yl)ethan-1-one (15 g, quant.) as a white solid. ESI-MS [M+H]⁺: 321.0.

### Synthesis of 1-(4-amino-6-chloropyridin-3-yl)ethan-1-one

1-(6-chloro-4-((2,4-dimethoxybenzyl)amino)pyridin-3-yl)ethan-1-one (6.7 g, 21 mmol) in TFA (50 mL) was stirred at 60° C. for 4 h. The reaction mixture was concentrated in vacuo, and the residue was diluted in NaHCO₃ (sat. aq., 80 mL). The resulting precipitate was collected by filtration, and the filtrate was extracted with EtOAc (60 mL×2). The combined organics were washed with brine (120 mL), dried over anhydrous Na₂SO₄ then concentrated in vacuo to give the crude product, which was combined with the above precipitate and purified by column chromatography on silica gel, eluting with 20˜40% EtOAc in PE to give 1-(4-amino-6-chloropyridin-3-yl)ethan-1-one (2.8 g, 78%) as a yellow solid. ESI-MS [M+H]⁺: 171.0.

### Synthesis of (1S,2S)—N-(5-acetyl-2-chloropyridin-4-yl)-2-(4-methylpyrimidin-2-yl)cyclopropane-1-carboxamide

To a stirred solution of 1-(4-amino-6-chloropyridin-3-yl)ethan-1-one (2.3 g, 13 mmol) and pyridine (10 g, 0.13 mol) in THF (30 mL) was added dropwise a solution of (1S,2S)-2-(4-methylpyrimidin-2-yl)cyclopropane-1-carbonyl chloride (2.9 g, 15 mmol) in THF (20 mL) at room temperature. The mixture was stirred at 70° C. for 18 h. The reaction mixture was concentrated in vacuo and diluted in water (50 mL). The resulting precipitate was collected by filtration, and the filtrate was extracted with EtOAc (60 mL×2). The combined organics were washed with brine (120 mL), dried over anhydrous Na₂SO₄ and concentrated in vacuo to give the crude product, which was combined with the above precipitate and purified by column chromatography on silica gel, eluting with 20˜40% EtOAc in PE to give the desired product (3.6 g, 84%) as a yellow solid. ESI-MS [M+H]⁺: 331.1.

### Synthesis of 7-chloro-2-((1S,2S)-2-(4-methylpyrimidin-2-yl)cyclopropyl)-1,6-naphthyridin-4 (1H)-one

A mixture of (1S,2S)—N-(5-acetyl-2-chloropyridin-4-yl)-2-(4-methylpyrimidin-2-yl)cyclopropane-1-carboxamide (3.6 g, 11 mmol) and NaOH (1.8 g, 45 mmol) in 1,4-dioxane (40 mL) was stirred at 110° C. for 1 h. The reaction mixture was cooled to room temperature and concentrated in vacuo. The residue was diluted with water (60 mL) and acidified to pH=4˜5 with HCl (3 M aq.) The precipitate was collected and dried in vacuo to give 7-chloro-2-((1S,2S)-2-(4-methylpyrimidin-2-yl)cyclopropyl)-1,6-naphthyridin-4 (1H)-one (2.8 g, 81%) as a yellow solid. ESI-MS [M+H]⁺: 313.1.

### Synthesis of 4,7-dichloro-2-((1S,2S)-2-(4-methylpyrimidin-2-yl)cyclopropyl)-1,6-naphthyridine

A mixture of 7-chloro-2-((1S,2S)-2-(4-methylpyrimidin-2-yl)cyclopropyl)-1,6-naphthyridin-4 (1H)-one (0.32 g, 1.0 mmol) in POCl₃ (6 mL) was stirred at 50° C. for 1 h. The reaction mixture was concentrated in vacuo and diluted with water (30 mL), pH of the mixture was adjusted to 9˜10 by NaHCO₃ (sat. aq.) and extracted with EtOAc (30 mL×3). The combined organics were washed with brine (60 mL), dried over anhydrous Na₂SO₄, concentrated in vacuo to give the crude product, which was purified by column chromatography on silica gel, eluting with 10˜20% EtOAc in PE to 4,7-dichloro-2-((1S,2S)-2-(4-methylpyrimidin-2-yl)cyclopropyl)-1,6-naphthyridine (0.23 g, 69%) as a yellow solid. ESI-MS [M+H]⁺: 331.0.

### Synthesis of 6-(7-chloro-2-((1S,2S)-2-(4-methylpyrimidin-2-yl)cyclopropyl)-1,6-naphthyridin-4-yl)-2-oxa-6-azaspiro[3.3]heptane

A mixture of 4,7-dichloro-2-((1S,2S)-2-(4-methylpyrimidin-2-yl)cyclopropyl)-1,6-naphthyridine (0.23 g, 0.69 mmol) and 2-oxa-6-azaspiro[3.3]heptane (0.34 g, 3.4 mmol) in NMP (3.0 mL) was stirred at 120° C. for 30 min in a microwave reactor. The reaction mixture was cooled to room temperature and poured into water (30 mL) and extracted with EtOAc (30 mL×3). The combined organics were washed with water (50 mL×3) and brine (50 mL), dried over anhydrous Na₂SO₄ then concentrated in vacuo to give the crude product, which was purified by column chromatography on silica gel, eluting with 0˜10% MeOH in DCM to give 6-(7-chloro-2-((1S,2S)-2-(4-methylpyrimidin-2-yl)cyclopropyl)-1,6-naphthyridin-4-yl)-2-oxa-6-azaspiro[3.3]heptane (0.27 g, quant.) as a white solid.

ESI-MS [M+H]⁺: 394.2.

### Synthesis of 1-(6-cyclopropyl-2-(((2-((1S,2S)-2-(4-methylpyrimidin-2-yl)cyclopropyl)-4-(2-oxa-6-azaspiro[3.3]heptan-6-yl)-1,6-naphthyridin-7-yl)oxy)methyl)imidazo[1,2-a]pyridin-8-yl)-3-methylimidazolidine-2,4-dione

A mixture of 6-(7-chloro-2-((1S,2S)-2-(4-methylpyrimidin-2-yl)cyclopropyl)-1,6-naphthyridin-4-yl)-2-oxa-6-azaspiro[3.3]heptane (0.24 g, 0.61 mmol), 1-(6-cyclopropyl-2-(hydroxymethyl)imidazo[1,2-a]pyridin-8-yl)-3-methylimidazolidine-2,4-dione (0.18 g, 0.61 mmol), [Pd(cinnamyl)Cl]₂ (62 mg, 0.12 mmol), RockPhos (0.11 g, 0.24 mmol) and Cs₂CO₃ (0.59 g, 1.8 mmol) in 1,4-dioxane (10 mL) was stirred at 90° C. for 14 h under N₂. The reaction mixture was filtered through Celite® and the filter cake was washed with DCM/MeOH (10/1, 20 mL). The filtrate was concentrated in vacuo to give the crude product, which was purified by column chromatography on silica gel, eluting with 0˜10% MeOH in DCM and Prep-HPLC to give 1-(6-cyclopropyl-2-(((2-((1S,2S)-2-(4-methylpyrimidin-2-yl)cyclopropyl)-4-(2-oxa-6-azaspiro[3.3]heptan-6-yl)-1,6-naphthyridin-7-yl)oxy)methyl)imidazo[1,2-a]pyridin-8-yl)-3-methylimidazolidine-2,4-dione (40 mg, 10%) as a white solid.

ESI-MS [M+H]⁺: 658.3. ¹H NMR (400 MHz, DMSO) δ 8.93 (s, 1H), 8.52 (d, J=5.1 Hz, 1H), 8.28 (d, J=1.1 Hz, 1H), 7.92 (s, 1H), 7.37 (d, J=1.5 Hz, 1H), 7.18 (d, J=5.1 Hz, 1H), 6.91 (s, 1H), 6.23 (s, 1H), 5.50 (s, 2H), 4.89 (s, 2H), 4.80-4.70 (m, 4H), 4.58 (s, 4H), 2.97 (s, 3H), 2.77-2.73 (m, 1H), 2.63-2.58 (m, 1H), 2.41 (s, 3H), 1.99-1.92 (m, 1H), 1.82-1.75 (m, 1H), 1.73-1.66 (m, 1H), 0.97-0.93 (m, 2H), 0.69-0.64 (m, 2H).

### Example 104

### Synthesis of rac-1-(2-(((5-methoxy-2-((1S*,2S*)-2-(4-methylpyrimidin-2-yl)cyclopropyl)quinolin-7-yl)amino methyl-6-methylimidazo[1,2-a]pyridin-8-yl-3-methylimidazolidine-2,4-dione (I-104)

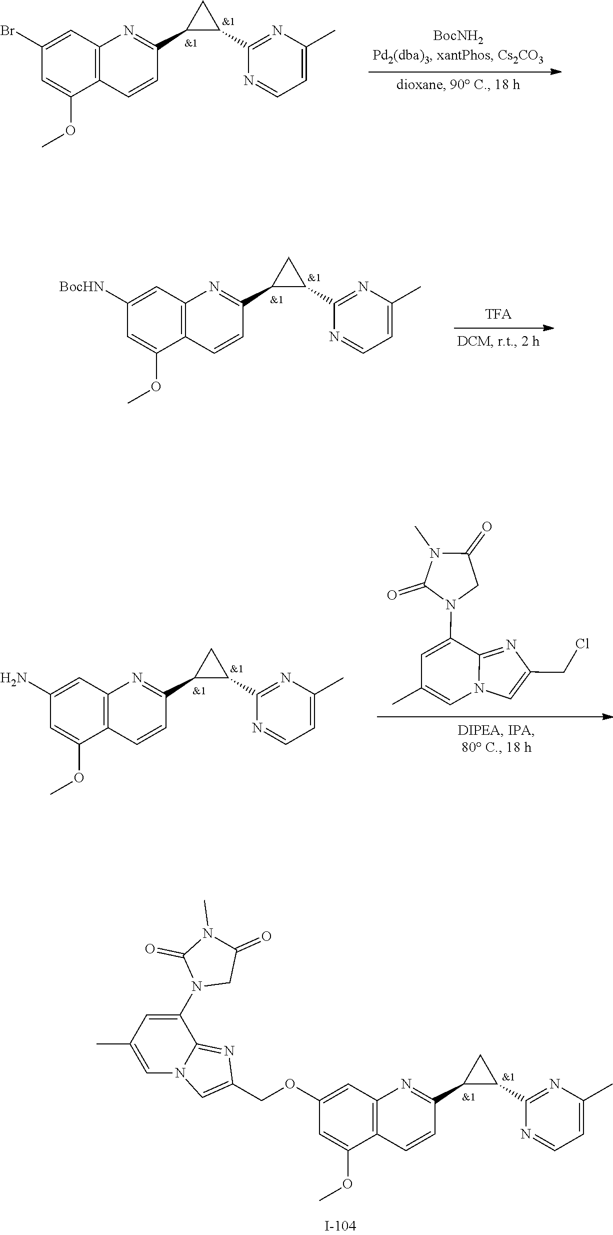

### Synthesis of rac-tert-butyl (5-methoxy-2-((1S*,2S*)-2-(4-methylpyrimidin-2-yl)cyclopropyl)quinolin-7-yl)carbamate

A mixture of rac-7-bromo-5-methoxy-2-((1S*,2S*)-2-(4-methylpyrimidin-2-yl)cyclopropyl)quinoline (140 mg, 0.38 mmol), tert-butyl carbamate (67 mg, 0.57 mmol), Pd₂(dba)₃ (35 mg, 0.038 mmol), XantPhos (46 mg, 0.08 mmol) and Cs₂CO₃ (372 mg, 1.14 mmol) in 1,4-dioxane (10 mL) was stirred at room temperature at 90° C. for 18 h under N₂. The reaction mixture was cooled to room temperature and filtered through Celite® and the filter cake was washed with DCM/MeOH (10/1, 50 mL). The filtrate was concentrated in vacuo to give the crude, which was purified by column chromatography (eluent: EtOAc/PE from 0 to 30%) to rac-tert-butyl (5-methoxy-2-((1S*,2S*)-2-(4-methylpyrimidin-2-yl)cyclopropyl)quinolin-7-yl)carbamate (120 mg, 78%) as a brown solid. ESI-MS [M+H]+: 407.2

### Synthesis of rac-5-methoxy-2-((1S*,2S*)-2-(4-methylpyrimidin-2-yl)cyclopropyl)quinolin-7-amine

rac-tert-butyl (5-methoxy-2-((1S*,2S*)-2-(4-methylpyrimidin-2-yl)cyclopropyl)quinolin-7-yl)carbamate (120 mg, 0.30 mmol) in DCM (10 mL) and TFA (1 mL) was stirred at room temperature for 2 h. The reaction was quenched with NaHCO₃ (sat. aq., 50 mL) and extracted with DCM (50 mL×3). The combined organic layers were washed with brine (50 mL), dried over Na₂SO₄, concentrated in vacuo to give the crude, which was purified by Prep-TLC (eluent: EtOAc/PE=1/2) to afford rac-5-methoxy-2-((1S*,2S*)-2-(4-methylpyrimidin-2-yl)cyclopropyl)quinolin-7-amine (70 mg, 76%) as a yellow solid. ESI-MS [M+H]+: 307.2

### Synthesis of rac-1-(2-(((5-methoxy-2-((1S*,2S*)-2-(4-methylpyrimidin-2-yl)cyclopropyl)quinolin-7-yl)amino)methyl)-6-methylimidazo[1,2-a]pyridin-8-yl)-3-methylimidazolidine-2,4-dione

A mixture of rac-5-methoxy-2-((1S*,2S*)-2-(4-methylpyrimidin-2-yl)cyclopropyl)quinolin-7-amine (45 mg, 0.15 mmol), 1-(2-(chloromethyl)-6-methylimidazo[1,2-a]pyridin-8-yl)-3-methylimidazolidine-2,4-dione (prepared in a similar manner to 1-(2-(chloromethyl)-6-cyclopropylimidazo[1,2-a]pyridin-8-yl)-3-methylimidazolidine-2,4-dione)(53 mg, 0.18 mmol) and DIPEA (58 mg, 0.45 mmol) in IPA (3 mL) was stirred at 80° C. for 18 h under N₂. The reaction was cooled to room temperature and diluted with water (50 mL), extracted with DCM (50 mL×3). The combined organic layers were washed with brine (50 mL), dried over Na₂SO₄, concentrated in vacuo to give the crude, which was purified by Prep-TLC (eluent: MeOH/DCM=1/15) to afford rac-1-(2-(((5-methoxy-2-((1S*,2S*)-2-(4-methylpyrimidin-2-yl)cyclopropyl)quinolin-7-yl)amino)methyl)-6-methylimidazo[1,2-a]pyridin-8-yl)-3-methylimidazolidine-2,4-dione (11 mg, 12%) as an off-white solid.

ESI-MS [M+H]+: 590.3. ¹H NMR (400 MHz, DMSO) δ 8.51 (d, J=5.1 Hz, 1H), 8.27-8.23 (m, 1H), 8.06 (d, J=8.3 Hz, 1H), 7.81 (s, 1H), 7.43 (d, J=1.4 Hz, 1H), 7.16 (d, J=5.1 Hz, 1H), 7.05 (d, J=8.4 Hz, 1H), 6.72 (t, J=4.9 Hz, 1H), 6.55 (d, J=1.7 Hz, 1H), 6.41 (d, J=1.3 Hz, 1H), 4.96 (s, 2H), 4.48 (d, J=5.6 Hz, 2H), 3.88 (s, 3H), 2.98 (s, 3H), 2.72-2.67 (m, 1H), 2.65-2.61 (m, 1H), 2.40 (s, 3H), 2.26 (s, 3H), 1.76-1.68 (m, 2H).

### Example 105

### Synthesis of 6-(((6-cyclopropyl-8-fluoroimidazo[1,2-a]pyridin-2-yl)methyl)amino)-3-((1S,2S)-2-(4-methylpyrimidin-2-yl)cyclopropyl)-4H-benzo[e][1,2,4]thiadiazine 1,1-dioxide (I-105)

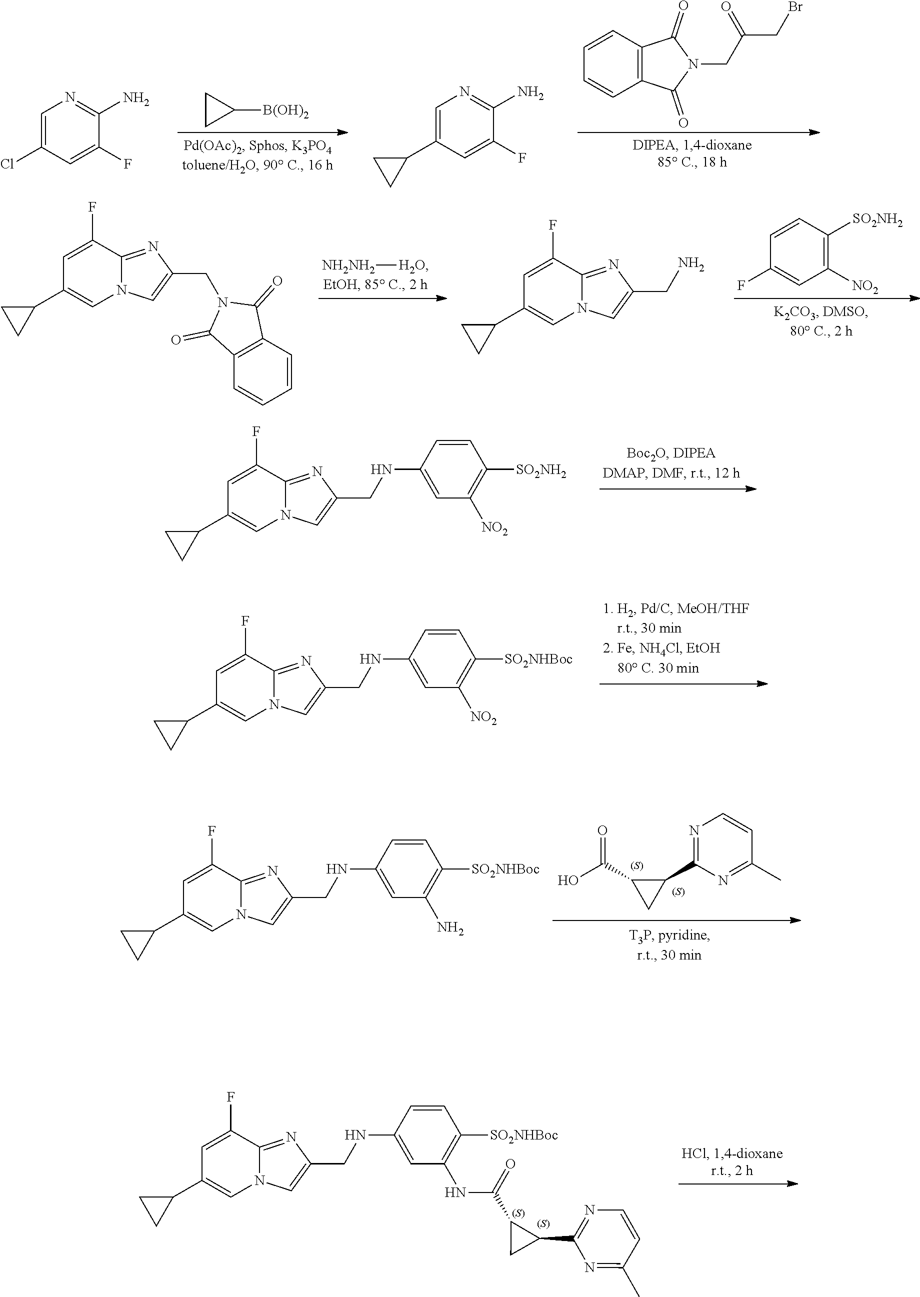

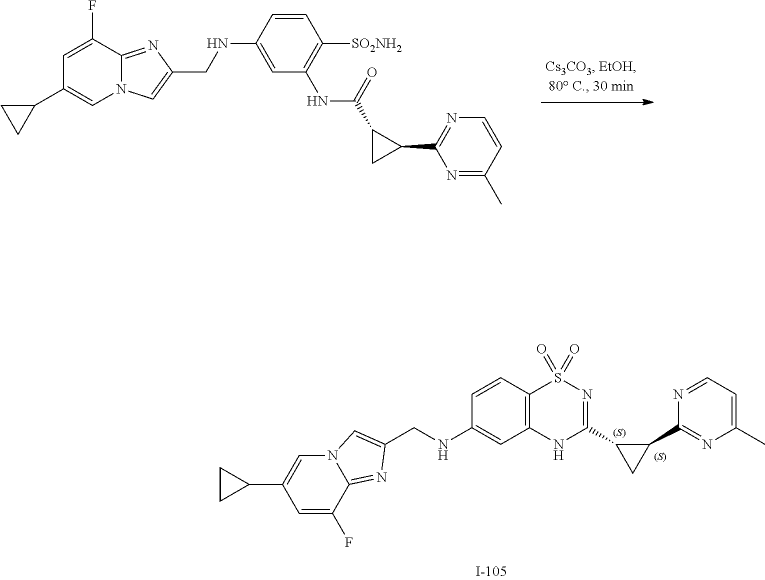

### Synthesis of 5-cyclopropyl-3-fluoropyridin-2-amine

To a solution of 5-chloro-3-fluoropyridin-2-amine (5 g, 34.2 mmol) in toluene/H₂O (100 mL/10 mL) was added cyclopropylboronic acid (4.4 g, 51.3 mmol), Pd(OAc)₂ (762 mg, 3.4 mmol), Sphos (2.79 g, 6.8 mmol) and K₃PO₄ (21.7 g, 102 mmol). After the reaction mixture was stirred at 90° C. for 16 h, the reaction was cooled to room temperature and filtered through Celite®. The filtrate was diluted with H₂O (100 mL), extracted with EtOAc (100 mL×3). The combined organic layers were washed with brine (100 mL), dried over Na₂SO₄ and concentrated in vacuo to give the crude, which was purified with column chromatography on silica gel, eluting with EtOAc/PE from 0-35% to give 5-cyclopropyl-3-fluoropyridin-2-amine (4.5 g, 87%) as a yellow oil. ESI-MS [M+H]+: 153.2.

### Synthesis of 2-((6-cyclopropyl-8-fluoroimidazo[1,2-a]pyridin-2-yl)methyl)isoindoline-1,3-dione

To a solution of 5-cyclopropyl-3-fluoropyridin-2-amine (4.5 g, 29.6 mmol) in 1,4-dioxane (80 mL) was added 2-(3-bromo-2-oxopropyl)isoindoline-1,3-dione (9.3 g, 33.0 mmol) and DIPEA (12.7 g, 98.6 mmol). The reaction mixture was stirred at 85° C. for 18 h. After cooling to room temperature, the reaction was diluted with water (100 mL) and extracted by EtOAc (80 mL×3). The combined organic layers were washed with brine (80 mL), dried over anhydrous Na₂SO₄ and concentrated in vacuo to give the crude, which was purified by silica gel chromatography, eluting with EtOAc/PE from 0-30% to give 2-((6-cyclopropyl-8-fluoroimidazo[1,2-a]pyridin-2-yl)methyl)isoindoline-1,3-dione (9 g, 91%) as a yellow solid. ESI-MS [M+H]+: 336.2.

### Synthesis of (6-cyclopropyl-8-fluoroimidazo[1,2-a]pyridin-2-yl)methanamine

To a solution of 2-((6-cyclopropyl-8-fluoroimidazo[1,2-a]pyridin-2-yl)methyl)isoindoline-1,3-dione (6.0 g, 17.9 mmol) in EtOH (80 mL) was added NH₂NH₂—H₂O (4.5 g, 71.6 mmol, 80% solution in H₂O). After the reaction mixture was stirred at 85° C. for 2 h, the reaction was cooled to room temperature and a white solid was precipitated. The mixture was filtered and the filtrant washed with EtOH (80 mL). The filtrate was concentrated in vacuo to give the crude, which was purified with silica gel chromatography, eluting with DCM/MeOH from 0-10% to give (6-cyclopropyl-8-fluoroimidazo[1,2-a]pyridin-2-yl)methanamine (3.4 g, 93%) as a yellow solid. ESI-MS [M+H]+: 206.2.

### Synthesis of 4-(((6-cyclopropyl-8-fluoroimidazo[1,2-a]pyridin-2-yl)methyl)amino)-2-nitrobenzenesulfonamide

To a solution of 4-fluoro-2-nitrobenzenesulfonamide (506 mg, 2.3 mmol) in DMSO (15 mL) was added (6-cyclopropyl-8-fluoroimidazo[1,2-a]pyridin-2-yl)methanamine (471 mg, 2.3 mmol) and K₂CO₃ (938 mg, 6.8 mmol). The resulting reaction solution was stirred at 80° C. for 2 h. After cooled to room temperature, the reaction was diluted with water (50 mL), extracted with EtOAc (50 mL×3). The combined organic layers were washed with brine (60 mL), dried over anhydrous Na₂SO₄, concentrated in vacuo to give the crude, which was purified with silica gel column chromatography, eluting with MeOH/DCM from 0-7% to give 4-(((6-cyclopropyl-8-fluoroimidazo[1,2-a]pyridin-2-yl)methyl)amino)-2-nitrobenzenesulfonamide (300 mg, 32%) as a yellow solid. ESI-MS [M+H]+: 406.2

### Synthesis of tert-butyl ((4-(((6-cyclopropyl-8-fluoroimidazo[1,2-a]pyridin-2-yl)methyl)amino)-2-nitrophenyl)sulfonyl)carbamate

To a solution of 4-(((6-cyclopropyl-8-fluoroimidazo[1,2-a]pyridin-2-yl)methyl)amino)-2-nitrobenzenesulfonamide (300 mg, 0.74 mmol) in DMF (10 mL) was added Boc₂O (323 mg, 1.48 mmol), DMAP (45 mg, 0.37 mmol) and DIPEA (380 mg, 2.96 mmol). The reaction was stirred at room temperature for 12 h, then diluted with water (30 mL) and extracted with EtOAc (30 mL×3). The combined organic layers were washed with brine (40 mL), dried over anhydrous Na₂SO₄, concentrated in vacuo to give the crude, which was purified with preparative TLC (eluent: DCM/MeOH=20/1) to give tert-butyl ((4-(((6-cyclopropyl-8-fluoroimidazo[1,2-a]pyridin-2-yl)methyl)amino)-2-nitrophenyl)sulfonyl)carbamate (250 mg, 67%) as a yellow solid. ESI-MS [M+H]+: 505.3.

### Synthesis of tert-butyl ((2-amino-4-(((6-cyclopropyl-8-fluoroimidazo[1,2-a]pyridin-2-yl)methyl)amino)phenyl)sulfonyl)carbamate

A mixture of tert-butyl ((4-(((6-cyclopropyl-8-fluoroimidazo[1,2-a]pyridin-2-yl)methyl)amino)-2-nitrophenyl)sulfonyl)carbamate (170 mg, 0.34 mmol) and Pd/C (50 mg) in MeOH/THF (5 mL/5 mL) was stirred at room temperature for 30 min under H₂ atmosphere. Then the reaction mixture was filtered through the Celite® and the filter cake washed with MeOH (30 mL). The filtrate was concentrated in vacuo to give the crude, which was redissolved in EtOH (10 mL). Fe (94 mg, 1.68 mmol) and NH₄Cl (180 mg, 3.36 mmol) were added thereto. The resulting reaction mixture was stirred at 80° C. for 30 min. After cooled to room temperature, the reaction mixture was filtered through the Celite®, washed with EtOH (40 mL). The filtrate was concentrated in vacuo to give the crude, which was purified by preparative TLC (eluent: DCM/MeOH=10:1) to give tert-butyl ((2-amino-4-(((6-cyclopropyl-8-fluoroimidazo[1,2-a]pyridin-2-yl)methyl)amino)phenyl)sulfonyl)carbamate (100 mg, 62%) as a yellow solid. ESI-MS [M+H]+: 476.2.

### Synthesis of tert-butyl ((4-(((6-cyclopropyl-8-fluoroimidazo[1,2-a]pyridin-2-yl)methyl)amino)-2-((1S,2S)-2-(4-methylpyrimidin-2-yl)cyclopropane-1-carboxamido)phenyl)sulfonyl)carbamate

To a solution of tert-butyl ((2-amino-4-(((6-cyclopropyl-8-fluoroimidazo[1,2-a]pyridin-2-yl)methyl)amino)phenyl)sulfonyl)carbamate (90 mg, 0.19 mmol) in pyridine (2 mL) was added (1S,2S)-2-(4-methylpyrimidin-2-yl)cyclopropane-1-carboxylic acid (34 mg, 0.19 mmol) and T₃P (1.2 g, 1.9 mmol, 50% in EtOAc). The resulting reaction mixture was stirred at room temperature for 30 min under N₂. The reaction was diluted with water (15 mL) and extracted with EtOAc (20 mL×3). The combined organic layers were washed with brine (30 mL), dried over anhydrous Na₂SO₄ and concentrated in vacuo to give the crude, which was purified by preparative TLC (eluent: DCM/MeOH=10/1) to give tert-butyl ((4-(((6-cyclopropyl-8-fluoroimidazo[1,2-a]pyridin-2-yl)methyl)amino)-2-((1S,2S)-2-(4-methylpyrimidin-2-yl)cyclopropane-1-carboxamido)phenyl)sulfonyl)carbamate (70 mg, 58%) as a yellow solid. ESI-MS [M+H]+: 636.2.

### Synthesis of (1S,2S)—N-(5-(((6-cyclopropyl-8-fluoroimidazo[1,2-a]pyridin-2-yl)methyl)amino)-2-sulfamoylphenyl)-2-(4-methylpyrimidin-2-yl)cyclopropane-1-carboxamide

To a solution of tert-butyl ((4-(((6-cyclopropyl-8-fluoroimidazo[1,2-a]pyridin-2-yl)methyl)amino)-2-((1S,2S)-2-(4-methylpyrimidin-2-yl)cyclopropane-1-carboxamido)phenyl)sulfonyl)carbamate (70 mg, 0.11 mmol) in MeOH (2 mL) was added HCl (1 mL, 4 mmol, 4 M in 1,4-dioxane). After the reaction mixture was stirred at room temperature for 2 h, the reaction was concentrated in vacuo to give (1S,2S)—N-(5-(((6-cyclopropyl-8-fluoroimidazo[1,2-a]pyridin-2-yl)methyl)amino)-2-sulfamoylphenyl)-2-(4-methylpyrimidin-2-yl)cyclopropane-1-carboxamide (70 mg, crude) as a yellow oil, which was used without further purification. ESI-MS [M+H]+: 536.2.

### Synthesis of 6-(((6-cyclopropyl-8-fluoroimidazo[1,2-a]pyridin-2-yl)methyl)amino)-3-((1S,2S)-2-(4-methylpyrimidin-2-yl)cyclopropyl)-4H-benzo[e][1,2,4]thiadiazine 1,1-dioxide

To a solution of (1S,2S)—N-(5-(((6-cyclopropyl-8-fluoroimidazo[1,2-a]pyridin-2-yl)methyl)amino)-2-sulfamoylphenyl)-2-(4-methylpyrimidin-2-yl)cyclopropane-1-carboxamide (70 mg, crude) in EtOH (4 mL) was added Cs₂CO₃ (97.8 mg, 0.3 mmol) and the reaction mixture was stirred at 80° C. for 30 min. After cooling to room temperature, the reaction mixture was filtered, washed with EtOH (15 mL). The filtrate was concentrated in vacuo to give the crude, which was purified with preparative TLC (eluent: DCM/MeOH=10/1) to give 6-(((6-cyclopropyl-8-fluoroimidazo[1,2-a]pyridin-2-yl)methyl)amino)-3-((1S,2S)-2-(4-methylpyrimidin-2-yl)cyclopropyl)-4H-benzo[e][1,2,4]thiadiazine 1,1-dioxide (30 mg, 53% over 2 steps) as a yellow solid.

ESI-MS [M+H]⁺: 518.2. ¹H NMR (400 MHz, DMSO) δ 11.93 (s, 1H), 8.55 (d, J=5.1 Hz, 1H), 8.20 (d, J=0.7 Hz, 1H), 7.73 (d, J=3.0 Hz, 1H), 7.42 (d, J=8.8 Hz, 1H), 7.34-7.12 (m, 2H), 6.89 (dd, J=12.5, 1.1 Hz, 1H), 6.73 (dd, J=8.8, 2.1 Hz, 1H), 6.25 (d, J=1.9 Hz, 1H), 4.40 (d, J=5.7 Hz, 2H), 2.68-2.58 (m, 1H), 2.47-2.44 (m, 1H), 2.42 (s, 3H), 1.94-1.87 (m, 1H), 1.74-1.61 (m, 2H), 0.97-0.84 (m, 2H), 0.72-0.58 (m, 2H).

### Example 106

### Synthesis of rac-1-(6-cyclopropyl-2-(((2-((1S*,2S*)-2-(4-methylpyrimidin-2-yl)cyclopropyl)quinolin-7-yl)amino)methyl)imidazo[1,2-a]pyrazin-8-yl)-3-methylimidazolidine-2,4-dione (I-106)

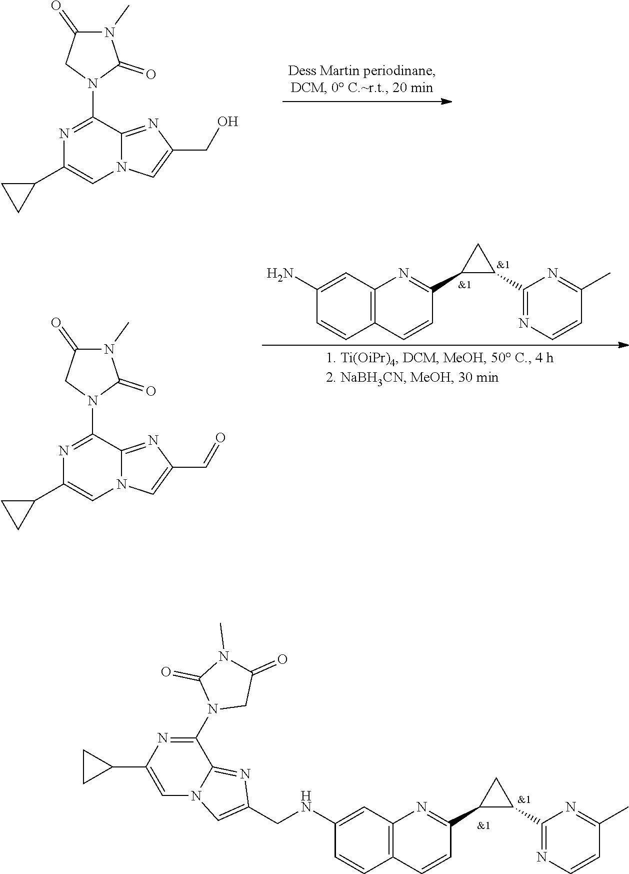 [SMILES: CN1C(=O)CN(c2nc(C3CC3)cn3cc(CO)nc23)C1=O ; CN1C(=O)CN(c2nc(C3CC3)cn3cc(C=O)nc23)C1=O ; Cc1ccnc([C@H]2C[C@@H]2c2ccc3ccc(N)cc3n2)n1 ; Cc1ccnc([C@H]2C[C@@H]2c2ccc3ccc(NCc4cn5cc(C6CC6)nc(N6CC(=O)N(C)C6=O)c5n4)cc3n2)n1]

### Synthesis of 6-cyclopropyl-8-(3-methyl-2,4-dioxoimidazolidin-1-yl)imidazo[1,2-a]pyrazine-2-carbaldehyde

To the mixture of 1-(6-cyclopropyl-2-(hydroxymethyl)imidazo[1,2-a]pyrazin-8-yl)-3-methylimidazolidine-2,4-dione (100 mg, 0.33 mmol) in DCM (10 mL) was added Dess Martin periodinane (348 mg, 0.82 mmol) at 0° C. The reaction mixture was stirred at room temperature for 20 min. After completed, the reaction was quenched with Na₂S₂O₃ (sat. aq., 10 mL), NaHCO₃ (sat., aq., 10 mL), and extracted by DCM (30 mL×3). The combined organic layers were concentrated in vacuo to give the crude, which was purified with preparative TLC(eluent: DCM/MeOH=10/1) to afford 6-cyclopropyl-8-(3-methyl-2,4-dioxoimidazolidin-1-yl)imidazo[1,2-a]pyrazine-2-carbaldehyde (50 mg, 51%) as yellow oil. ESI-MS [M+H]+: 300.1.

### Synthesis of 1-(6-cyclopropyl-2-(((2-(2-(4-methylpyrimidin-2-yl)cyclopropyl)quinolin-7-yl)amino)methyl)imidazo[1,2-a]pyrazin-8-yl)-3-methylimidazolidine-2,4-dione

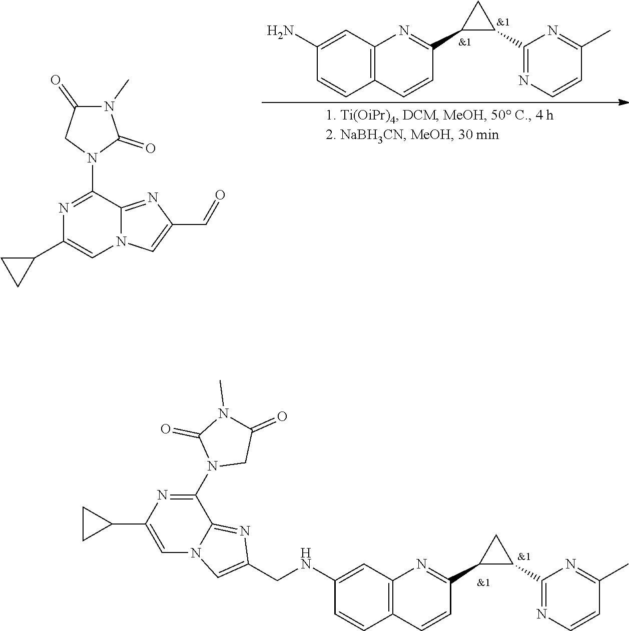 [SMILES: CN1C(=O)CN(c2nc(C3CC3)cn3cc(C=O)nc23)C1=O ; Cc1ccnc([C@H]2C[C@@H]2c2ccc3ccc(N)cc3n2)n1 ; Cc1ccnc([C@H]2C[C@@H]2c2ccc3ccc(NCc4cn5cc(C6CC6)nc(N6CC(=O)N(C)C6=O)c5n4)cc3n2)n1]

To the mixture of 6-cyclopropyl-8-(3-methyl-2,4-dioxoimidazolidin-1-yl)imidazo[1,2-a]pyrazine-2-carbaldehyde (40 mg, 0.13 mmol), rac-2-((1S*,2S*)-2-(4-methylpyrimidin-2-yl)cyclopropyl)quinolin-7-amine (36 mg, 0.13 mmol) in DCM (4 mL) and MeOH (4 mL) was added Ti(OiPr)₄ (190 mg, 0.67 mmol). The reaction solution was stirred at 50° C. for 4 h under N₂. After cooled to room temperature, NaBH₃CN (25 mg, 0.39 mmol) was added and stirred at room temperature for 30 min. The mixture was quenched with H₂O (2 mL), filtered through Celite® and the filter cake was washed with (DCM/MeOH=10:1, 10 mL). The filtrate was diluted with H₂O (10 mL) and extracted with DCM (15 mL×3). The combined organic layers were washed with brine (20 mL), dried over Na₂SO₄ then concentrated in vacuo. The residue was purified by column chromatography on silica gel eluting with a gradient of 20-100% EtOAc in PE to rac-1-(6-cyclopropyl-2-(((2-((1S*,2S*)-2-(4-methylpyrimidin-2-yl)cyclopropyl)quinolin-7-yl)amino)methyl)imidazo[1,2-a]pyrazin-8-yl)-3-methylimidazolidine-2,4-dione (29 mg, 39%) as a yellow solid.

ESI-MS [M+H]+: 560.3. Purity: 97.8% (214 nm), 98.5% (254 nm), ¹H NMR (400 MHz, DMSO) δ 8.51 (d, J=5.1 Hz, 1H), 8.37 (s, 1H), 7.93-7.91 (m, 2H), 7.60-7.58 (m, 1H), 7.17 (d, J=5.1 Hz, 1H), 7.13 (d, J=5.2 Hz, 1H), 7.07-7.04 (m, 1H), 6.90 (t, J=5.9 Hz, 1H), 6.75 (d, J=2.0 Hz, 1H), 4.91 (s, 2H), 4.55 (d, J=5.8 Hz, 2H), 2.97 (s, 3H), 2.72-2.66 (m, 2H), 2.41 (s, 3H), 2.02-1.97 (m, 1H), 1.76-1.68 (m, 2H), 0.87-0.85 (m, 4H).

### Example 107

### Synthesis of 1-(5-cyclopropyl-2-(((2-((1S,2S)-2-(4-methylpyrimidin-2-yl)cyclopropyl)-4-(2-oxa-6-azaspiro[3.3]heptan-6-yl)quinolin-7-yl)amino)methyl)pyrazolo[1,5-a]pyridin-7-yl)-3-methylimidazolidine-2,4-dione (I-107)

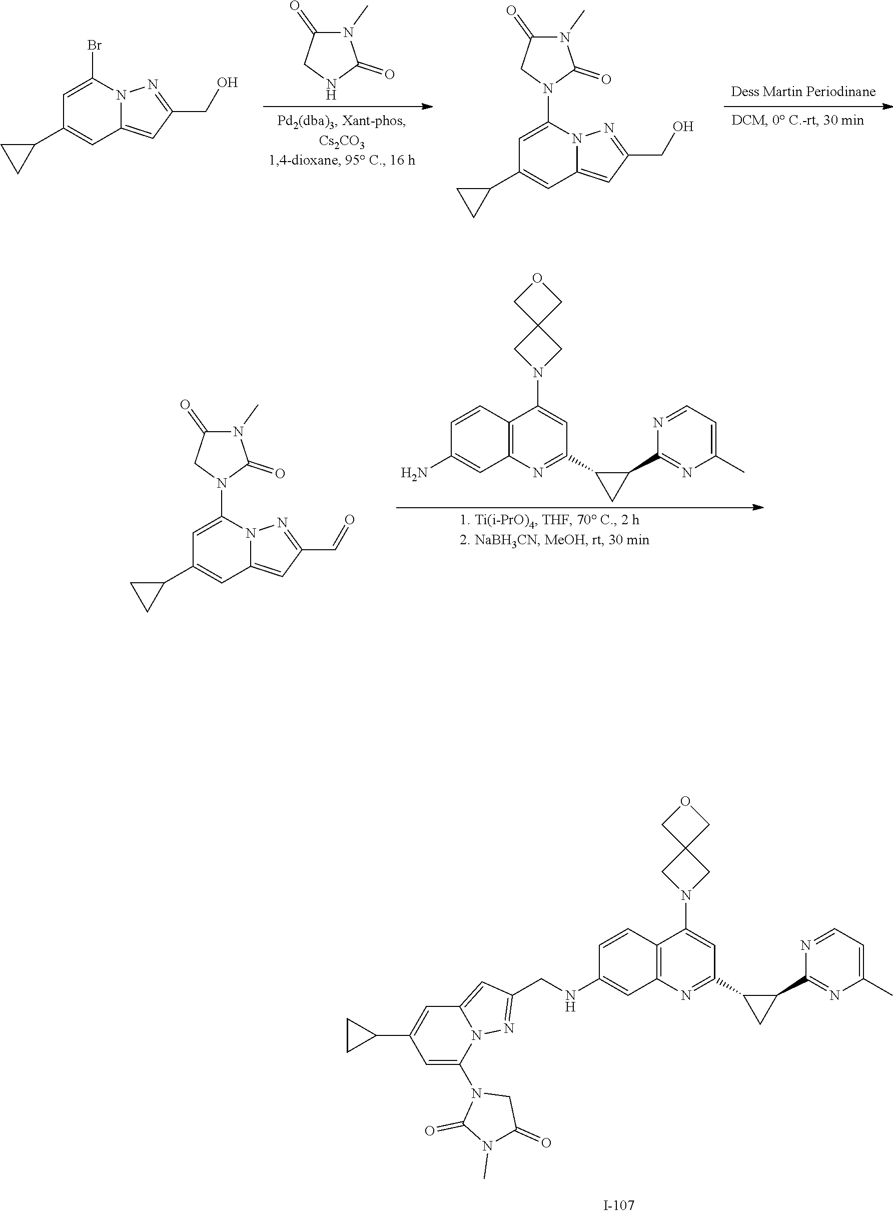

### Synthesis of 1-(5-cyclopropyl-2-(hydroxymethyl)pyrazolo[1,5-a]pyridin-7-yl)-3-methylimidazolidine-2,4-dione

To a mixture of (7-bromo-5-cyclopropylpyrazolo[1,5-a]pyridin-2-yl)methanol (266 mg, 1.0 mmol) and 3-methylimidazolidine-2,4-dione (342 mg, 3.0 mmol) in dioxane (10 mL) was Pd₂(dba)₃ (92 mg, 0.1 mmol), Xantphos (12 mg, 0.2 mmol) and Cs₂CO₃ (978 mg, 3 mmol) under N₂. After stirring at 90° C. for 16 h under N₂, the reaction mixture was cooled to room temperature, filtered and the filter cake was washed with DCM/MeOH (10/1, 50 mL). The filtrate was concentrated in vacuo to give the crude, which was purified by column chromatography on silica gel, eluting with 3-10% MeOH in DCM to give 1-(5-cyclopropyl-2-(hydroxymethyl)pyrazolo[1,5-a]pyridin-7-yl)-3-methylimidazolidine-2,4-dione (180 mg, 60%). ESI-MS [M+H]+: 301.1

### Synthesis of 5-cyclopropyl-7-(3-methyl-2,4-dioxoimidazolidin-1-yl)pyrazolo[1,5-a]pyridine-2-carbaldehyde

To a mixture of 1-(5-cyclopropyl-2-(hydroxymethyl)pyrazolo[1,5-a]pyridin-7-yl)-3-methylimidazolidine-2,4-dione (170 mg, 0.57 mmol) in DCM (15 mL) was added Dess Martin periodinane (593.6 mg, 1.4 mmol) at 0° C. The reaction mixture was stirred at room temperature for 30 min. The reaction was diluted with DCM (30 mL) then washed by NaHCO₃ (sat. aq., 20 mL) and Na₂S₂O₃ (sat.aq., 20 mL). The organic layer was washed with brine and concentrated in vacuo. The residue was purified by column chromatography on silica gel eluting with a gradient of 0-50% EtOAc in PE to afford 5-cyclopropyl-7-(3-methyl-2,4-dioxoimidazolidin-1-yl)pyrazolo[1,5-a]pyridine-2-carbaldehyde (80 mg, 47%) as a yellow solid. ESI-MS [M+H]+: 299.1.

### Synthesis of 1-(5-cyclopropyl-2-(((2-((1S,2S)-2-(4-methylpyrimidin-2-yl)cyclopropyl)-4-(2-oxa-6-azaspiro [3.3]heptan-6-yl)quinolin-7-yl)amino)methyl)pyrazolo[1,5-a]pyridin-7-yl)-3-methyl imidazolidine-2,4-dione

To a mixture of 5-cyclopropyl-7-(3-methyl-2,4-dioxoimidazolidin-1-yl)pyrazolo[1,5-a]pyridine-2-carbaldehyde (26.4 mg, 0.088 mmol) and 2-((1S,2S)-2-(4-methylpyrimidin-2-yl)cyclopropyl)-4-(2-oxa-6-azaspiro[3.3]heptan-6-yl)quinolin-7-amine (33 mg, 0.088 mmol) in THF (5 mL) was added Ti(i-PrO)₄ (125 mg, 0.44 mmol). The reaction mixture was stirred at 70° C. for 2 h. After cooling to room temperature, MeOH (4 mL) was added to the reaction, followed by NaBH₃CN (16 mg, 0.26 mmol). The reaction mixture was stirred at room temperature for another 30 min. The mixture was quenched by H₂O (10 mL), extracted with EtOAc (30 mL×3). The combined organic layers were washed with brine (15 mL), dried over anhydrous Na₂SO₄, and concentrated in vacuo to give the crude product, which was purified by preparative TLC(eluent: MeOH/DCM=15/1) to 1-(5-cyclopropyl-2-(((2-((1S,2S)-2-(4-methylpyrimidin-2-yl)cyclopropyl)-4-(2-oxa-6-azaspiro[3.3]heptan-6-yl)quinolin-7-yl)amino)methyl)pyrazolo[1,5-a]pyridin-7-yl)-3-methylimidazolidine-2,4-dione (24 mg, 42%) as a yellow solid.

ESI-MS [M+H]+: 656.3. ¹H NMR (400 MHz, DMSO) δ 8.56 (d, J=5.1 Hz, 1H), 7.77-7.68 (m, 2H), 7.37 (d, J=1.7 Hz, 1H), 7.25 (d, J=5.1 Hz, 1H), 6.97 (d, J=7.9 Hz, 1H), 6.77-6.75 (m, 2H), 6.47 (s, 1H), 5.88 (s, 1H), 4.76-4.52 (m, 12H), 2.97 (s, 3H), 2.85-2.80 (m, 1H), 2.74-2.70 (m, 1H), 2.43 (s, 3H), 2.02-1.97 (m, 1H), 1.92-1.87 (m, 1H), 1.81-1.76 (m, 1H), 1.05-1.00 (m, 2H), 0.75-0.71 (m, 2H).

### Example 108

### Synthesis of 1-(5-cyclopropyl-2-(((2-((1S,2S)-2-(4-methylpyrimidin-2-yl)cyclopropyl)-4-(2-oxa-6-azaspiro[3.3]heptan-6-yl)quinolin-7-yl)oxy)methyl)pyrazolo[1,5-a]pyridin-7-yl)-3-methyl imidazolidine-2,4-dione (I-108)

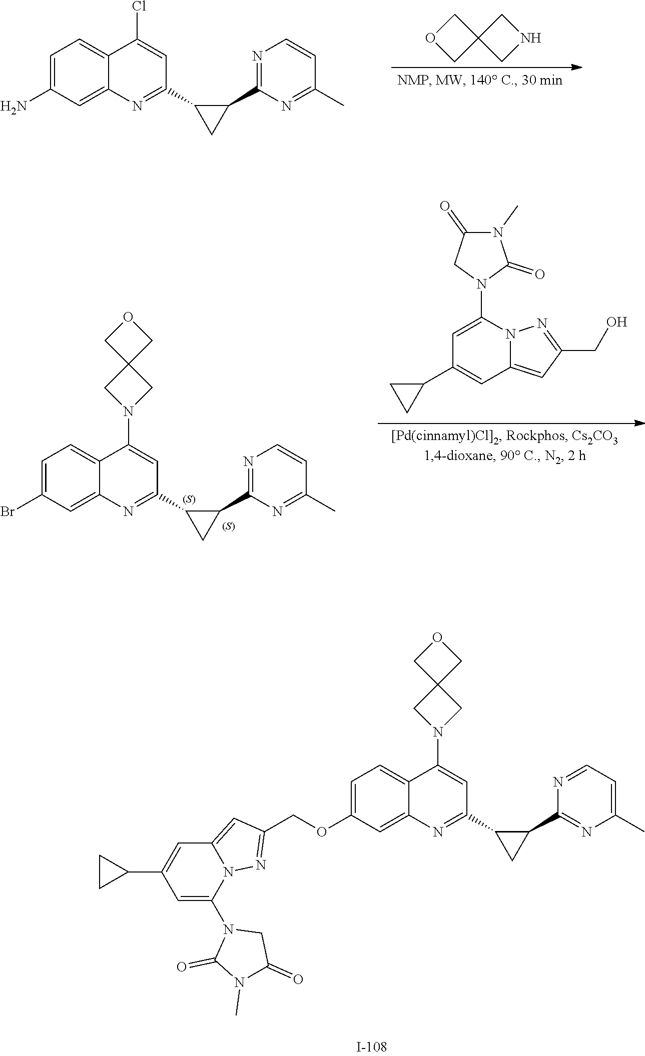

### Synthesis of 6-(7-bromo-2-((1S,2S)-2-(4-methylpyrimidin-2-yl)cyclopropyl)quinolin-4-yl)-2-oxa-6-azaspiro[3.3]heptane

A mixture of 7-bromo-4-chloro-2-((1S,2S)-2-(4-methylpyrimidin-2-yl)cyclopropyl)quinoline (1.7 g, 4.5 mmol) and 2-oxa-6-azaspiro[3.3]heptane (2.3 g, 23 mmol) in NMP (10 mL) was stirred at 140° C. in a microwave reactor for 0.5 h. The mixture was cooled to room temperature, diluted water (100 mL) and then extracted with EtOAc (100 mL×3). The combined organic layers were washed with brine (60 mL), dried over Na₂SO₄, filtered and concentrated in vacuo. The residue was purified by flash column chromatography (eluent: MeOH/DCM=0-6%) to afford 6-(7-bromo-2-((1S,2S)-2-(4-methylpyrimidin-2-yl)cyclopropyl)quinolin-4-yl)-2-oxa-6-azaspiro[3.3]heptane (1.87 g, yield 95%) as a white solid.

ESI-MS [M+H]+: 437.1

### Synthesis of 1-(5-cyclopropyl-2-(((2-((1S,2S)-2-(4-methylpyrimidin-2-yl)cyclopropyl)-4-(2-oxa-6-azaspiro[3.3]heptan-6-yl)quinolin-7-yl)oxy)methyl)pyrazolo[1,5-a]pyridin-7-yl)-3-methyl imidazolidine-2,4-dione

To a mixture of 1-(5-cyclopropyl-2-(hydroxymethyl)pyrazolo[1,5-a]pyridin-7-yl)-3-methylimidazolidine-2,4-dione (50 mg, 0.17 mmol) and 6-(7-bromo-2-((1S,2S)-2-(4-methylpyrimidin-2-yl)cyclopropyl)quinolin-4-yl)-2-oxa-6-azaspiro[3.3]heptane (74 mg, 0.17 mmol) in 1,4-dioxane (5 mL) was added [Pd(cinnamyl)Cl]₂ (17 mg, 0.033 mmol), Rockphos (31 mg, 0.067 mmol) and Cs₂CO₃ (160 mg, 0.50 mmol). The reaction mixture was stirred at 90° C. for 2 h under N₂ then cooled to room temperature, diluted with DCM (20 mL) and filtered through Celite®. The filter cake was washed with DCM/MeOH (v/v=3/1, 50 mL) then the filtrate was concentrated in vacuo. The residue was purified by preparative TLC (elute: DCM/MeOH=10/1) to afford 1-(5-cyclopropyl-2-(((2-((1S,2S)-2-(4-methylpyrimidin-2-yl)cyclopropyl)-4-(2-oxa-6-azaspiro[3.3]heptan-6-yl)quinolin-7-yl)oxy)methyl)pyrazolo[1,5-a]pyridin-7-yl)-3-methylimidazolidine-2,4-dione (8.3 mg, yield: 7%) as a white solid.

ESI-MS [M+H]+: 657.3, ¹H NMR (400 MHz, DMSO) δ 8.52 (d, J=5.1 Hz, 1H), 7.81 (s, 1H), 7.44 (d, J=1.7 Hz, 1H), 7.28 (s, 1H), 7.18 (d, J=5.0 Hz, 1H), 7.03 (s, 1H), 6.82 (d, J=1.8 Hz, 1H), 6.67 (s, 1H), 6.22 (s, 1H), 5.38 (s, 2H), 4.74 (s, 4H), 4.65 (s, 2H), 4.49 (s, 4H), 3.00 (s, 3H), 2.77-2.70 (m, 1H), 2.64-2.58 (m, 1H), 2.41 (s, 3H), 2.05-2.00 (m, 1H), 1.83-1.76 (m, 1H), 1.72-1.66 (m, 1H), 1.06-1.01 (m, 2H), 0.79-0.75 (m, 2H).

### Example 109

### Inhibitory Activity of Exemplary Compounds Against Plasma Kallikrein.

Compounds were evaluated for inhibition of the human activated kallikrein enzyme in two formats of an assay employing a fluorogenic peptide substrate. In one assay format, the concentrations of reagents were as follows: 20 mM Tris pH 7.5, 1 mM EDTA, 150 mM sodium chloride, 0.1% PEG-400, 0.1% Triton X-100, 500 pM activated kallikrein enzyme, 300 uM Pro-Phe-Arg-7-amido-4-methylcoumarin (PFR-AMC) substrate. Prior to reaction initiation with substrate, enzyme and inhibitors were preincubated for 30 min at room temperature. After initiation with substrate, reactions were incubated for 10 min at room temperature and fluorescence emission at 460 nm from 380 nm excitation measured with a microplate reader. In another assay format, the concentrations of reagents were as follows: 20 mM Tris pH 7.5, 1 mM EDTA, 150 mM sodium chloride, 0.1% PEG-400, 0.1% Triton X-100, 5 pM activated kallikrein enzyme, 300 uM PFR-AMC substrate. Prior to reaction initiation with substrate, enzyme and inhibitors were preincubated for 30 min at room temperature. After initiation with substrate, reactions were incubated for 18 hr at room temperature and fluorescence emission at 460 nm from 380 nm excitation measured with a microplate reader.

Table 6 provides the results of the assay in the format with 500 pM activated kallikrein assay. For the compounds listed in Table 6, the EC₅₀ values are reported according to the following ranges: A≤1.0 nM; 1.0 nM<B≤10 nM; 10 nM<C≤100 nM; 100 nM<D≤6500 nM; 6500 nM<E.

TABLE 6

| | Primary Dose Response Assay: |
|---|---|
| Compound I-# | Average EC50 |
| 1 | A |
| 2 | C |
| 3 | B |
| 4 | C |
| 5 | B |
| 6 | B |
| 7 | A |
| 8 | A |
| 9 | A |
| 10 | A |
| 11 | A |
| 12 | A |
| 13 | B |
| 14 | B |
| 15 | B |
| 16 | D |
| 17 | D |
| 18 | C |
| 19 | B |
| 20 | B |
| 21 | A |
| 22 | B |
| 23 | A |
| 24 | D |
| 25 | A |
| 26 | B |
| 27 | A |
| 28 | A |
| 29 | A |
| 30 | A |
| 31 | A |
| 32 | A |
| 33 | A |
| 34 | B |
| 35 | A |
| 36 | A |
| 37 | A |
| 38 | A |
| 39 | A |
| 40 | A |
| 41 | A |
| 42 | B |
| 43 | A |
| 44 | E |
| 45 | E |
| 46 | E |
| 47 | E |
| 48 | C |
| 49 | B |
| 50 | C |
| 51 | B |
| 53 | B |
| 54 | B |
| 55 | A |
| 56 | E |
| 57 | A |
| 58 | C |
| 59 | A |
| 60 | B |
| 61 | A |
| 62 | D |
| 63 | D |
| 64 | A |
| 65 | A |
| 66 | C |
| 67 | B |
| 68 | B |
| 69 | A |
| 70 | E |
| 71 | E |
| 72 | D |
| 73 | C |
| 74 | A |
| 75 | A |
| 76 | A |
| 77 | A |
| 78 | A |
| 79 | A |
| 80 | A |
| 81 | A |
| 82 | B |
| 83 | B |
| 84 | A |
| 85 | A |
| 86 | A |
| 87 | A |
| 88 | A |
| 89 | A |
| 90 | B |
| 91 | A |
| 92 | A |
| 93 | A |
| 94 | D |
| 95 | A |
| 96 | A |
| 97 | A |
| 98 | A |
| 99 | B |
| 100 | A |
| 101 | A |
| 102 | C |
| 103 | A |
| 104 | B |
| 105 | A |
| 106 | B |
| 107 | A |
| 108 | A |

### Example 110

### Neat Human and Rat Plasma Assay

To analyze inhibition of plasma kallikrein in an ex vivo setting, the potency of compounds was measure in contact pathway-activated plasma assays. In a fluorogenic peptide substrate assay, test compounds dissolved in DMSO were added to sodium citrate collected human or rat plasma in a 96-well microplate. Alternatively, citrated plasma was collected from rats administered the compounds orally or by IV. 10 nM of human FXIIa (Enzyme Research Laboratories) diluted in pKal buffer (20 mM Tris-HCl, pH 7.5, 150 mM NaCl, 1 mM EDTA, 0.1% PEG-8000, and 0.1% Triton X-100) was added to the plasma, followed by the 100 μM of the profluorescent, synthetic plasma kallikrein substrate PFR-AMC (also diluted in pKal buffer). Final plasma concentration in the reaction was 78%. Fluorescence was immediately monitored by excitation/emission wavelengths of 360 nm/480 nm respectively over a period of 5 minutes in a microplate reader. The resulting linear increase in fluorescence emission (reflecting pKal proteolysis of PFR-AMC substrate) was fit to extract a proteolytic rate (fluorescent units over time), and this rate was subsequently plotted against compound inhibitor concentration. Resulting plots were fit to a standard 4-parameter IC50/IC90 equation to determine min/max values, IC50/90, and slope. All experimental steps were performed at room temperature. Table 7 provides results of the assay.

For the compounds listed in Table 7, the IC₉₀ values are reported according to the following ranges: A≤400 nM; 400 nM<B≤1,500 nM; 1,500 nM<C≤10,000 nM; 10,000 nM<D≤22,000 nM.

TABLE 7

| Compound I-# | Human Plasma Assay: IC90 |
|---|---|
| 1 | B |
| 5 | C |
| 6 | C |
| 7 | B |
| 8 | C |
| 9 | C |
| 10 | A |
| 11 | B |
| 12 | B |
| 13 | A |
| 14 | C |
| 15 | C |
| 20 | D |
| 21 | C |
| 23 | A |
| 25 | C |
| 26 | C |
| 27 | B |
| 28 | B |
| 29 | A |
| 30 | A |
| 31 | A |
| 32 | A |
| 33 | B |
| 34 | C |
| 35 | A |
| 36 | A |
| 37 | A |
| 38 | A |
| 39 | A |
| 40 | A |
| 41 | A |
| 42 | A |
| 43 | A |
| 51 | C |
| 54 | C |
| 55 | C |
| 57 | C |
| 59 | C |
| 60 | D |
| 61 | B |
| 64 | B |
| 65 | B |
| 67 | B |
| 68 | C |
| 69 | B |
| 74 | C |
| 75 | B |
| 77 | B |
| 78 | C |
| 79 | A |
| 80 | C |
| 81 | C |
| 82 | C |
| 84 | C |
| 85 | C |
| 86 | C |
| 87 | B |
| 88 | B |
| 89 | C |
| 91 | C |
| 92 | C |
| 93 | C |
| 95 | B |
| 96 | A |
| 97 | A |
| 98 | A |
| 99 | B |
| 100 | A |
| 101 | A |
| 103 | A |
| 104 | B |
| 105 | A |
| 106 | C |
| 107 | A |
| 108 | A |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of examples.

### Exemplary Enumerated Embodiments

1. A compound of formula (I):

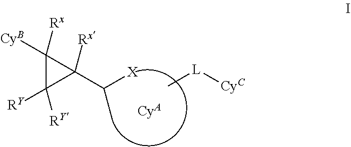

or a pharmaceutically acceptable salt thereof,
wherein:
Cy^{A} is an 8- to 10-membered bicyclic heteroarylene having 1-5 heteroatoms independently selected from oxygen, nitrogen, and sulfur, a 10- to 14-membered tricyclic heteroarylene having 1-6 heteroatoms independently selected from oxygen, nitrogen, and sulfur, an 8- to 14-membered saturated or partially unsaturated bicyclic heterocyclyl having 1-6 heteroatoms selected from oxygen, nitrogen, or sulfur, or a 10- to 15-membered saturated or partially unsaturated tricyclic heterocyclyl having 1-6 heteroatoms selected from oxygen, nitrogen, or sulfur, wherein Cy^{A} is substituted with 0-6 —R^{A} groups;
   each R^{A} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, 5- to 6-membered heteroaryl having 1-4 heteroatoms independently selected from oxygen, nitrogen, and sulfur, 3- to 7-membered saturated or partially unsaturated monocyclic carbocyclyl, 3- to 7-membered saturated or partially unsaturated monocyclic heterocyclyl having 1-2 heteroatoms selected from oxygen, nitrogen, or sulfur, or a 6- to 12-membered spirocyclic ring system having 0-4 heteroatoms independently selected from oxygen, nitrogen, and sulfur;
each R is independently hydrogen or an optionally substituted group selected from C₁₋₆ aliphatic, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur;
X is —N═ or —NR—, wherein X is adjacent to the ring atom of Cy^{A} attached to the cyclopropyl ring;
each R^{Y} and R^{Y′} is independently selected from hydrogen, halogen, and an optionally substituted C₁₋₆ aliphatic group;
each R^{x} and R^{x′} is independently selected from hydrogen, halogen, or —CN;
Cy^{B} is selected from phenyl, 8- to 10-membered bicyclic aryl, a 5- to 6-membered heteroaryl having 1-3 heteroatoms independently selected from oxygen, nitrogen, and sulfur, or a 7- to 10-membered heteroaryl having 1-4 heteroatoms independently selected from oxygen, nitrogen, and sulfur, wherein Cy^{B} is substituted with 0-5 —R^{B} groups; or
Cy^{B} and R^{x}, together with their intervening atoms, form a 6- to 12-membered spirocyclic ring system having 0-4 heteroatoms independently selected from oxygen, nitrogen, and sulfur, wherein the ring or rings formed by Cy^{B} and R^{x} may be substituted with 0-4 —R^{B} groups;
   each R^{B} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, a 3- to 7-membered saturated or partially unsaturated monocyclic heterocyclyl having 1-2 heteroatoms selected from oxygen, nitrogen, or sulfur, or a 5- to 6-membered heteroaryl having 1-4 heteroatoms independently selected from oxygen, nitrogen, and sulfur;
L is an optionally substituted C₁₋₃ hydrocarbon chain, wherein 1 to 3 methylene units are optionally and independently replaced with —O—, —NR^{z}—, —S—, —SO—, or —SO₂—; or L is an optionally substituted 5- to 6-membered saturated or partially unsaturated heterocyclene, having 1-3 heteroatoms independently selected from oxygen, nitrogen, and sulfur;
   each R^{z} is independently selected from hydrogen, —(CH₂)₀₋₃OR, —(CH₂)₀₋₃C(O)OR, or an optionally substituted C₁₋₆ aliphatic group;
Cy^{C} is a 7- to 10-membered bicyclic heteroaryl having 1-4 heteroatoms independently selected from oxygen, nitrogen, and sulfur, or a 6- to 12-membered saturated or partially unsaturated bicyclic heterocyclyl having 1-4 heteroatoms independently selected from oxygen, nitrogen, or sulfur, wherein Cy^{C} is substituted with 0-6-L^{C}-R^{C} groups;
   each L^{C} is independently selected from a covalent bond or an optionally substituted C₁₋₆ hydrocarbon chain, wherein 1 to 3 methylene units are optionally and independently replaced with —O— or —NR—;
   each R^{C} is independently selected from halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, Cy^{D}, or an optionally substituted group selected from C₁₋₆ aliphatic;
      each Cy^{D} is independently selected from a 3- to 7-membered saturated or partially unsaturated monocyclic carbocyclyl, a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, phenyl, a 6- to 12-membered saturated or partially unsaturated bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur, wherein Cy^{D} is substituted with 0-4 -L^{D}-R^{D} groups;
         each L^{D} is independently selected from a covalent bond or an optionally substituted C₁₋₆ hydrocarbon chain, wherein 1 to 3 methylene units are optionally and independently replaced with —O— or —NR—; and
each R^{D} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, a 3- to 7-membered saturated or partially unsaturated monocyclic carbocyclyl, or a 3- to 7-membered saturated or partially unsaturated monocyclic heterocyclyl having 1-2 heteroatoms selected from oxygen, nitrogen, or sulfur.

2. The compound of embodiment 1, wherein Cy^{A} is an 8- to 10-membered bicyclic heteroarylene having 1-5 heteroatoms independently selected from oxygen, nitrogen, and sulfur, wherein Cy^{A} is substituted with 0-4 —R^{A} groups.

3. The compound of any one of the preceding embodiments, wherein Cy^{A} is quinolinylene substituted with 0-4 —R^{A} groups.

4. The compound of any one of the preceding embodiments, wherein Cy^{A} is selected from the group consisting of:

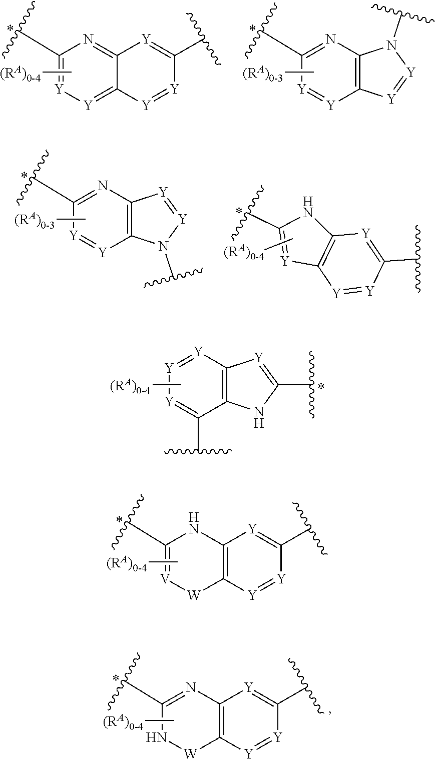

wherein:
each Y is independently selected from ═C(H)— or ═N—;
W is selected from —C(O)— or —S(O)₂—;
V is selected from ═CH— or ═N—;
and * represents point of attachment to the cyclopropyl ring.

5. The compound of any one of the preceding embodiments, wherein Cy^{A} is selected from the group consisting of:

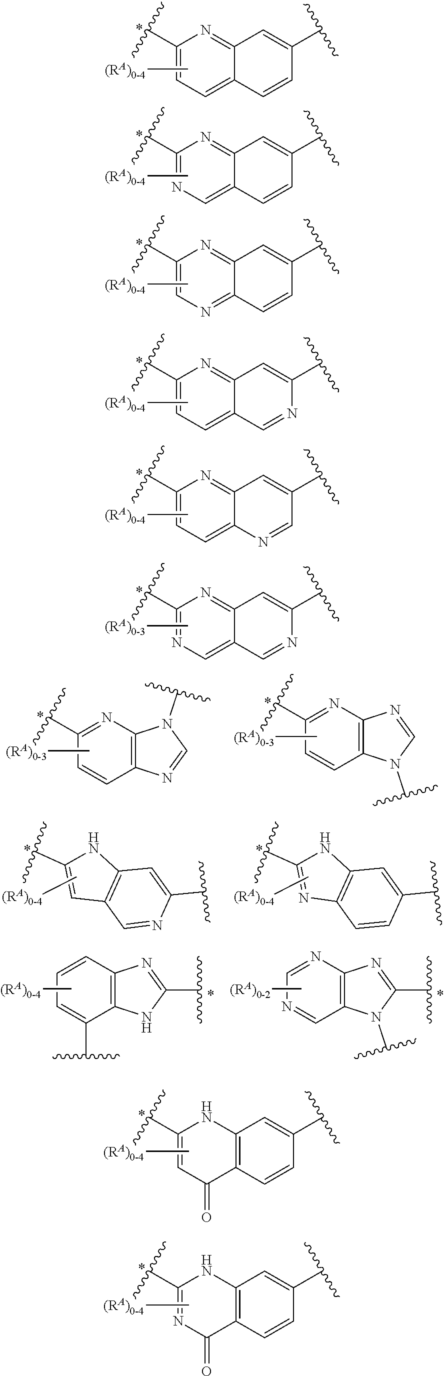

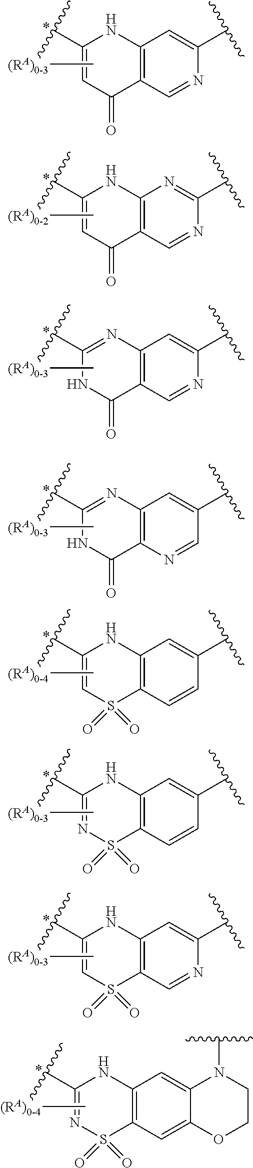

wherein * represents point of attachment to the cyclopropyl ring.

6. The compound of any one of the preceding embodiments, wherein Cy^{A} is selected from the group consisting of:

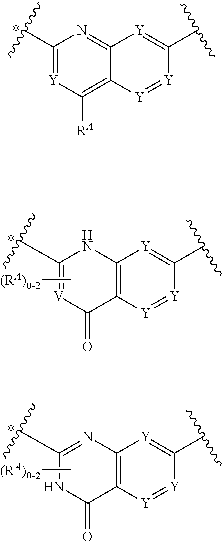

wherein:

* represents point of attachment to the cyclopropyl ring.

7. The compound of any one of the preceding embodiments, wherein Cy^{A} is selected from the group consisting of:

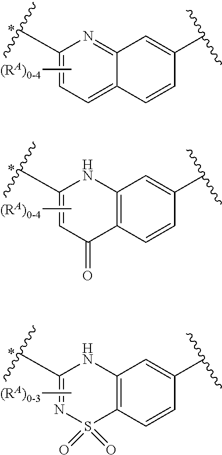

wherein * represents point of attachment to the cyclopropyl ring.

8. The compound of any one of the preceding embodiments, wherein Cy^{A} is selected from the group consisting of:

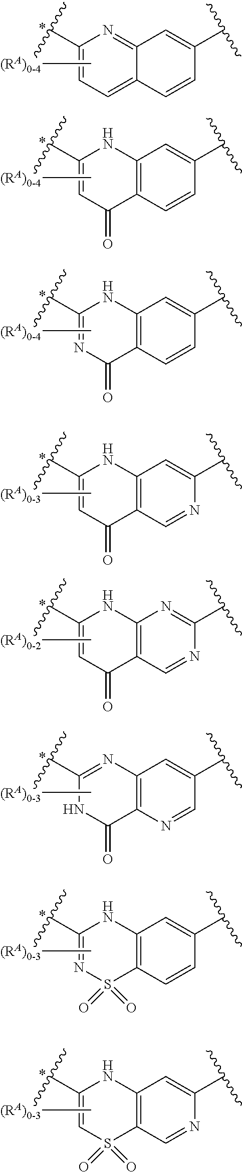

wherein * represents point of attachment to the cyclopropyl ring.

9. The compound of any one of the preceding embodiments, wherein Cy^{A} is selected from the group consisting of:

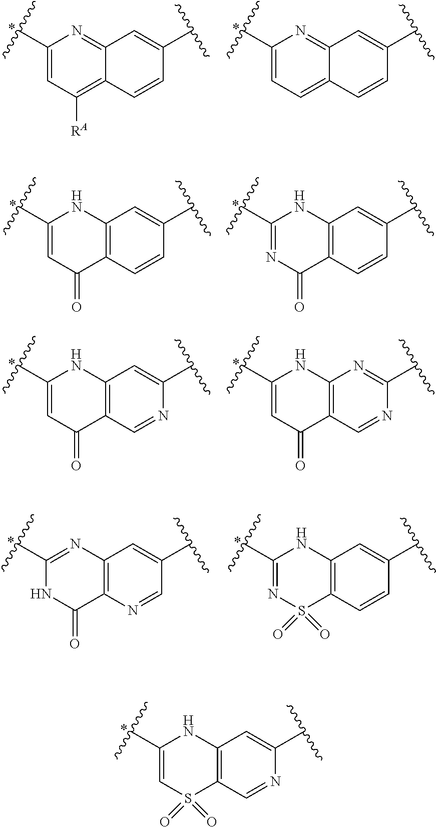

wherein * represents point of attachment to the cyclopropyl ring.

10. The compound of any one of the preceding embodiments, wherein each R^{A} is independently selected from oxo, halogen, —CN, —N(R)₂, —N(R)S(O)₂R, —OR, or an optionally substituted group selected from C₁₋₆ aliphatic, 5- to 6-membered heteroaryl having 1-4 heteroatoms independently selected from oxygen, nitrogen, and sulfur, 3- to 7-membered saturated or partially unsaturated monocyclic heterocyclyl having 1-2 heteroatoms selected from oxygen, nitrogen, or sulfur, or a 6- to 12-membered spirocyclic ring system having 0-4 heteroatoms independently selected from oxygen, nitrogen, and sulfur.

11. The compound of any one of the preceding embodiments, wherein X is —N═.

12. The compound of any one of the preceding embodiments, wherein X is —NH—.

13. The compound of any one of the preceding embodiments, wherein Cy^{B} is selected from phenyl or a 5- to 6-membered heteroaryl having 1-3 heteroatoms independently selected from oxygen, nitrogen, and sulfur, wherein Cy^{B} is substituted with 0-4 —R^{B} groups.

14. The compound of any one of the preceding embodiments, wherein Cy^{B} is phenyl, wherein Cy^{B} is substituted with 0-4 —R^{B}

15. The compound of any one of the preceding embodiments, wherein Cy^{B} is a 6-membered heteroaryl having 1-3 heteroatoms independently selected from oxygen, nitrogen, and sulfur, wherein Cy^{B} is substituted with 0-4 —R^{B} groups.

16. The compound of any one of the preceding embodiments, wherein Cy^{B} is a pyrimidinyl group or a pyridinyl group, wherein Cy^{B} is substituted with 0-2 —R^{B} groups.

17. The compound of any one of the preceding embodiments, wherein Cy^{B} is:

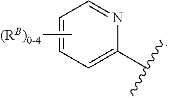

18. The compound of any one of the preceding embodiments, wherein Cy^{B} is

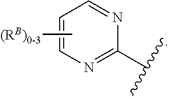

19. The compound of any one of the preceding embodiments, wherein Cy^{B} is

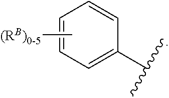

20. The compound of any one of the preceding embodiments, wherein each R^{B} is independently selected from halogen or an optionally substituted C₁₋₆ aliphatic

21. The compound of any one of the preceding embodiments, wherein each of R^{x} and R^{x′} is independently selected from hydrogen and halogen.

22. The compound of any one of the preceding embodiments, wherein each of R^{x} and R^{x′} is hydrogen.

23. The compound of any one of the preceding embodiments, wherein one of R^{x} and R^{x′} is hydrogen and the other is halogen.

24. The compound of any one of the preceding embodiments, wherein each of R and R^{Y′} is independently selected from hydrogen and halogen.

25. The compound of any one of the preceding embodiments, wherein each of R and R^{Y′} is hydrogen.

26. The compound of any one of the preceding embodiments, wherein L is an optionally substituted C₁₋₃ hydrocarbon chain, wherein 1-3 methylene units are optionally replaced with —O—, —NR^{z}—, —S—, or —SO₂—.

27. The compound of any one of the preceding embodiments, wherein L is an optionally substituted C₁₋₃ hydrocarbon chain, wherein 1 methylene unit is optionally replaced with —O—, —NR^{z}—, —S—, or —SO₂—.

28. The compound of any one of the preceding embodiments, wherein L is an optionally substituted C₁ hydrocarbon chain.

29. The compound of any one of the preceding embodiments, wherein L is an optionally substituted C₂ hydrocarbon chain, wherein 1 methylene unit is optionally replaced with —NR^{z}— or —O—.

30. The compound of any one of the preceding embodiments, wherein L is an optionally substituted C₂ hydrocarbon chain, wherein the methylene unit connected to Cy^{A} is replaced with —NR^{z}— or —O—.

31. The compound of any one of the preceding embodiments, wherein L is an optionally substituted C₂ hydrocarbon chain, wherein the methylene unit connected to Cy^{A} is replaced with —NR^{z}—.

32. The compound of embodiment 31, wherein L is *—NHCH(Me)-, wherein * represents the point of attachment to Cy^{A}.

33. The compound of embodiment 31, wherein L is *—NHCH₂—, wherein * represents the point of attachment to Cy^{A}.

34. The compound of any one of embodiments 1-30, wherein L is an optionally substituted C₂ hydrocarbon chain, wherein the methylene unit connected to Cy^{A} is replaced with —O—.

35. The compound of embodiment 34, wherein L is *—OCH(Me)-, wherein * represents the point of attachment to Cy^{A}.

36. The compound of embodiment 34, wherein L is *—OCH₂—, wherein * represents the point of attachment to Cy^{A}.

37. The compound of any one of the preceding embodiments, wherein L comprises a two-atom spacer between Cy^{A} and Cy^{C}.

38. The compound of any one of the preceding embodiments, wherein L is an optionally substituted 5-membered saturated or partially unsaturated heterocyclene, having 1 heteroatom independently selected from oxygen, nitrogen, and sulfur.

39. The compound of any one of the preceding embodiments, wherein L is optionally substituted

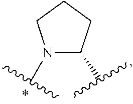 [SMILES: *[C@H]1CCCN1*]

wherein * represents the point of attachment to Cy^{A}.

40. The compound of any one of the preceding embodiments, wherein optional substituents on L are independently selected from —(CH₂)₀₋₄R^{∘}, —(CH₂)₀₋₄OR^{∘}, —(CH₂)₀₋₄OC(O)R^{∘}, and —(CH₂)₀₋₄N(R^{∘})₂, wherein each R^{∘} is independently as defined above and described in classes and subclasses herein.

41. The compound of any one of the preceding embodiments, wherein Cy^{C} is an 8- to 10-membered bicyclic aryl, wherein Cy^{C} is substituted with 0-6-L^{C}-R^{C} groups.

42. The compound of any one of the preceding embodiments, wherein Cy^{C} is selected from the group consisting of:

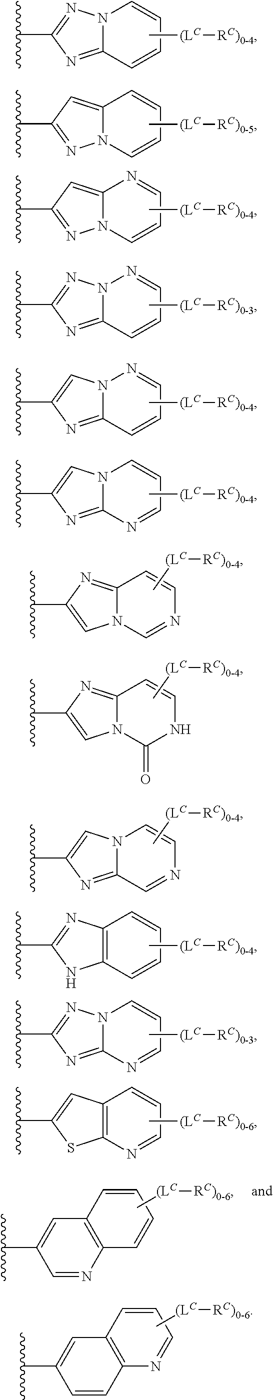

43. The compound of any one of the preceding embodiments, wherein Cy^{C} is selected from the group consisting of:

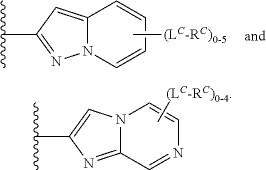

44. The compound of any one of the preceding embodiments, wherein the compound is of Formula II:

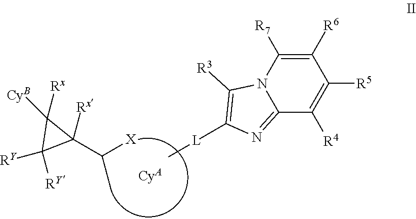

or a pharmaceutically acceptable salt thereof, wherein each R³, R⁴, R⁵, R⁶, and R⁷ is independently selected from hydrogen or -L^{C}-R^{C}.

45. The compound of any one of the preceding embodiments, wherein each of R³, R⁴, R⁵, R⁶, and R⁷ is independently selected from hydrogen or -L^{C}-R^{C}, wherein each L^{C} is independently selected from a covalent bond or an optionally substituted C₁₋₆ hydrocarbon chain, wherein 1 to 3 methylene units are optionally and independently replaced with —O— or —NR—; and wherein each R^{C} is independently selected from halogen, —CN, —C(O)R, —C(O)₂R, —C(O)N(R)₂, —N(R)₂, —N(R)C(O)R, —N(R)C(O)₂R, —N(R)S(O)₂R, —S(O)₂R, —S(O)₂N(R)₂, Cy^{D}, or an optionally substituted group selected from C₁₋₆ aliphatic.

46. The compound of any one of the preceding embodiments, wherein R³ is hydrogen.

47. The compound of any one of the preceding embodiments, wherein R⁴ is selected from hydrogen or L^{C}-R^{C}, wherein L^{C} is a covalent bond and wherein R^{C} is selected from halogen, —CN, —C(O)R, —C(O)₂R, —C(O)N(R)₂, —N(R)₂, —N(R)C(O)R, —N(R)C(O)₂R, —N(R)S(O)₂R, —OR, —S(O)₂R, —S(O)₂N(R)₂, Cy^{D}, or an optionally substituted group selected from C₁₋₆ aliphatic.

48. The compound of any one of the preceding embodiments, wherein Cy^{D} is a 5-membered saturated or partially unsaturated monocyclic heterocyclyl having 1-2 heteroatoms selected from oxygen, nitrogen, or sulfur.

49. The compound of any one of the preceding embodiments, wherein R⁴ is selected from the group consisting of:

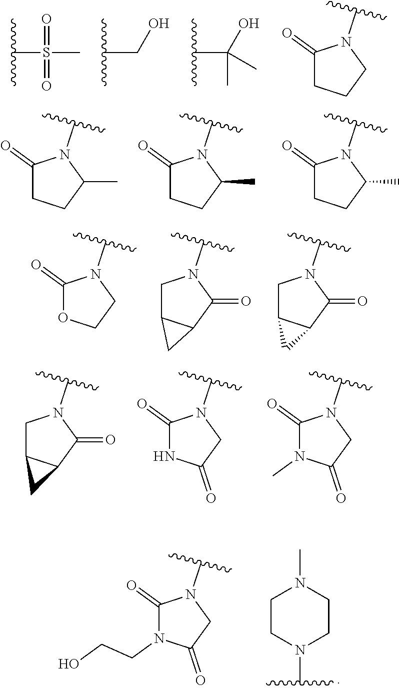

50. The compound of any one of the preceding embodiments, wherein R⁵ is hydrogen.

51. The compound of any one of the preceding embodiments, wherein R⁶ is selected from hydrogen or L^{C}-R^{C}, wherein L^{C} is a covalent bond, and wherein R^{C} is selected from halogen, —N(R)₂, —OR, Cy^{D}, or an optionally substituted C₁₋₆ aliphatic group.

52. The compound of any one of the preceding embodiments, wherein Cy^{D} is a cyclopropyl group substituted with 0-4 L^{D}-R^{D} groups. In some embodiments, L^{D} is a covalent bond and R^{D} is selected from halogen and optionally substituted C₁₋₆ aliphatic.

53. The compound of any one of the preceding embodiments, wherein Cy^{D} is:

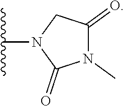 [SMILES: *N1CC(=O)N(C)C1=O]

54. The compound of any one of the preceding embodiments, wherein R⁷ is hydrogen.

55. The compound of any one of the preceding embodiments, wherein the compound is of Formula III:

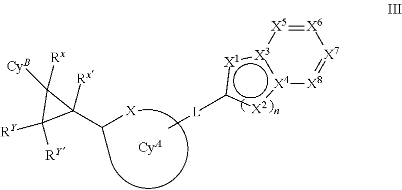

or a pharmaceutically acceptable salt thereof,
wherein X¹ is N, CH, or C-L^{C}-R^{C};
each X² is independently selected from N, CH, or C-L^{C}-R^{C};
X³ and X⁴ is independently N or C, wherein at least one of X³ or X⁴ is C;
each of X⁵, X⁶, X⁷, and X⁸ are independently selected from N, CH, or C-L^{C}-R^{C}; and
n is 1 or 2.

56. The compound of any one of the preceding embodiments, wherein the compound is of Formulae IV-a, IV-b, or IV-c:

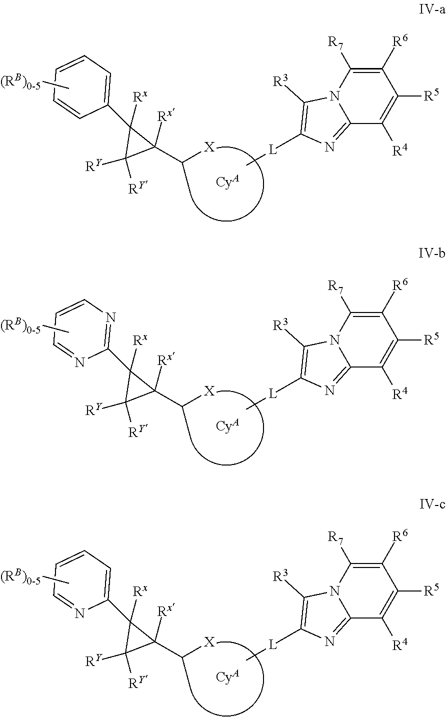

or a pharmaceutically acceptable salt thereof.

57. The compound of any one of the preceding embodiments, wherein the compound is of Formulae V-a, V-b, or V-c:

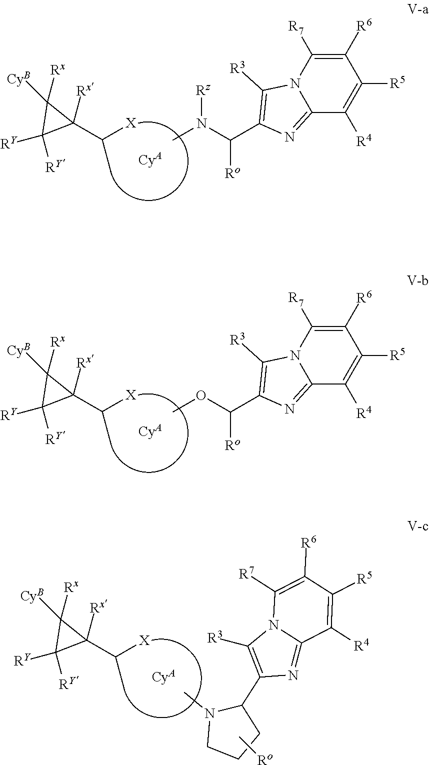

or a pharmaceutically acceptable salt thereof.

58. The compound of any one of the preceding embodiments, wherein R^{∘} is hydrogen or methyl.

59. The compound of any one of the preceding embodiments, wherein R^{∘} is hydrogen or —OH.

60. The compound of any one of the preceding embodiments, wherein the compound is of Formulae VI-a, VI-b, or VI-c:

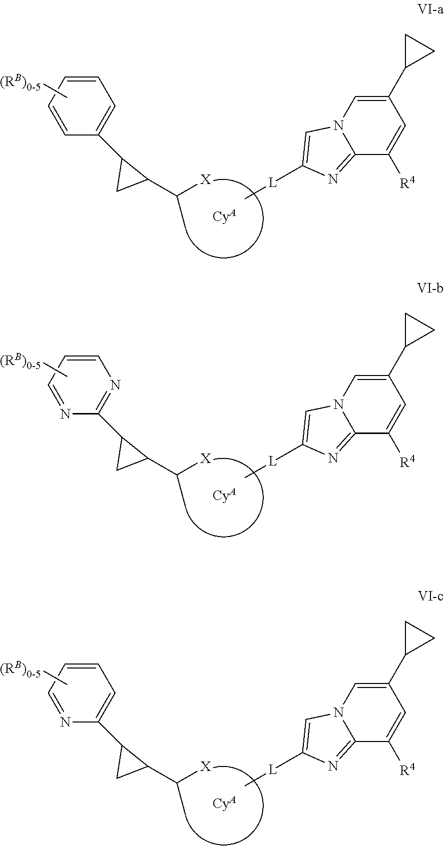

or a pharmaceutically acceptable salt thereof, and
R⁴ is hydrogen or L^{C}-R^{C}, wherein L^{C} is a covalent bond and R^{C} is halogen or Cy^{D}, wherein Cy^{D} is a 5-membered saturated or partially unsaturated monocyclic heterocyclyl having 1-2 heteroatoms selected from nitrogen, and wherein Cy^{D} is substituted with 0-4 -L^{D}-R^{D} groups.

61. The compound of any one of the preceding embodiments, wherein Cy^{D} is a ring selected from:

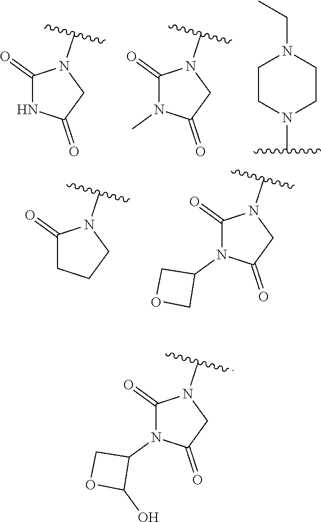

62. The compound of any one of the preceding embodiments, wherein the moiety:

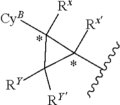

(including where one or more of R^{x}, R^{x′}, R^{Y}, or R^{Y′} is hydrogen) is in the relative trans configuration with respect to the Cy^{B} and Cy^{A} group attached to the two stereocenters marked with an *.

63. The compound of embodiment 62, wherein the compound is a racemic mixture with respect to the two stereocenters marked with an *.

64. The compound of any one of embodiments 1-62, wherein the absolute stereochemistry of the moiety:

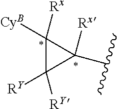

(including where one or more of R^{x}, R^{x′}, R^{Y}, or R^{Y′} is hydrogen) is as follows:

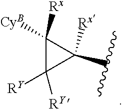

65. The compound of any one of embodiments 1-62, wherein the absolute stereochemistry of the moiety

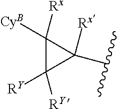

(including where one or more of R^{x}, R^{x′}, R^{Y}, or R^{Y′} is hydrogen) is as follows:

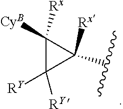

66. The compound of any one of the preceding embodiments, wherein each R^{∘} is independently selected from hydrogen, C₁₋₆ aliphatic, or —OH.

67. The compound of any one of the preceding embodiments, wherein R^{∘} of L is methyl.

68. The compound of any one of the preceding embodiments, wherein the compound is selected from compounds I-1 through I-108, or a pharmaceutically acceptable salt thereof.

69. A pharmaceutical composition comprising a compound of any one of the preceding embodiments.

70. The pharmaceutical composition comprising a compound of any one of the preceding embodiments, further comprising a pharmaceutically acceptable excipient.

71. The composition of embodiment 69 or 70, wherein the composition is suitable for oral administration.

72. A method of treating a plasma kallikrein-mediated disease or disorder using a compound or composition of any one of the preceding embodiments.

73. The method of embodiment 72, wherein the disease or disorder is hereditary angioedema.

74. The method of embodiment 72, wherein the disease or disorder is diabetic macular edema.

75. A method of treating hereditary angioedema comprising administering to a patient in need thereof a compound or composition of any one of the preceding embodiments.

76. A method of treating diabetic macular edema comprising administering to a patient in need thereof a compound or composition of any one of the preceding embodiments.

77. The method of any one of embodiments 73 or 75, wherein administration of the compound partially or completely inhibits, delays onset of, reduces severity of, and/or reduces incidence of one or more symptoms, features, and/or causes of a hereditary angioedema.

78. The method of embodiment 77, wherein the compound is administered orally.

1. A compound of formula (I): 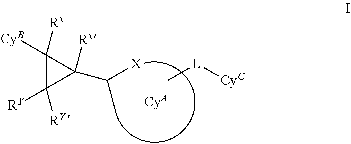
or a pharmaceutically acceptable salt thereof,
wherein:
Cy^{A} is an 8- to 10-membered bicyclic heteroarylene having 1-5 heteroatoms independently selected from oxygen, nitrogen, and sulfur, a 10- to 14-membered tricyclic heteroarylene having 1-6 heteroatoms independently selected from oxygen, nitrogen, and sulfur, an 8- to 14-membered saturated or partially unsaturated bicyclic heterocyclyl having 1-6 heteroatoms selected from oxygen, nitrogen, or sulfur, or a 10- to 15-membered saturated or partially unsaturated tricyclic heterocyclyl having 1-6 heteroatoms selected from oxygen, nitrogen, or sulfur, wherein Cy^{A} is substituted with 0-6 —R^{A} groups;
each R^{A} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, phenyl, 5- to 6-membered heteroaryl having 1-4 heteroatoms independently selected from oxygen, nitrogen, and sulfur, 3- to 7-membered saturated or partially unsaturated monocyclic carbocyclyl, 3- to 7-membered saturated or partially unsaturated monocyclic heterocyclyl having 1-2 heteroatoms selected from oxygen, nitrogen, or sulfur, or a 6- to 12-membered spirocyclic ring system having 0-4 heteroatoms independently selected from oxygen, nitrogen, and sulfur;
each R is independently hydrogen or an optionally substituted group selected from C₁₋₆ aliphatic, 3- to 7-membered saturated or partially unsaturated monocyclic carbocyclyl, or 3- to 7-membered saturated or partially unsaturated monocyclic heterocyclyl having 1-2 heteroatoms selected from oxygen, nitrogen, or sulfur;
X is —N═ or —NR—, wherein X is adjacent to the ring atom of Cy^{A} attached to the cyclopropyl ring;
each R^{Y} and R^{Y′} is independently selected from hydrogen, halogen, and an optionally substituted C₁₋₆ aliphatic group;
each R^{x} and R^{x′} is independently selected from hydrogen, halogen, or —CN;
Cy^{B} is selected from phenyl, 8- to 10-membered bicyclic aryl, a 5- to 6-membered heteroaryl having 1-3 heteroatoms independently selected from oxygen, nitrogen, and sulfur, or a 7- to 10-membered heteroaryl having 1-4 heteroatoms independently selected from oxygen, nitrogen, and sulfur, wherein Cy^{B} is substituted with 0-5 —R^{B} groups; or
Cy^{B} and R^{x}, together with their intervening atoms, form a 6- to 12-membered spirocyclic ring system having 0-4 heteroatoms independently selected from oxygen, nitrogen, and sulfur, wherein the ring or rings formed by Cy^{B} and R^{x} may be substituted with 0-4 —R^{B} groups;
each R^{B} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, or an optionally substituted group selected from C₁₋₆ aliphatic, a 3- to 7-membered saturated or partially unsaturated monocyclic heterocyclyl having 1-2 heteroatoms selected from oxygen, nitrogen, or sulfur, or a 5- to 6-membered heteroaryl having 1-4 heteroatoms independently selected from oxygen, nitrogen, and sulfur;
L is an optionally substituted C₁₋₃ hydrocarbon chain, wherein 1 to 3 methylene units are optionally and independently replaced with —O—, —NR^{z}—, —S—, —SO—, or —SO₂—; or L is an optionally substituted 5- to 6-membered saturated or partially unsaturated heterocyclene, having 1-3 heteroatoms independently selected from oxygen, nitrogen, and sulfur;
each R^{z} is independently selected from hydrogen, —(CH₂)₀₋₃OR, —(CH₂)₀₋₃C(O)OR, or an optionally substituted C₁₋₆ aliphatic group;
Cy^{C} is a 7- to 10-membered bicyclic heteroaryl having 1-4 heteroatoms independently selected from oxygen, nitrogen, and sulfur, or a 6- to 12-membered saturated or partially unsaturated bicyclic heterocyclyl having 1-4 heteroatoms independently selected from oxygen, nitrogen, or sulfur, wherein Cy^{C} is substituted with 0-6-L^{C}-R^{C} groups;
each L^{C} is independently selected from a covalent bond or an optionally substituted C₁₋₆ hydrocarbon chain, wherein 1 to 3 methylene units are optionally and independently replaced with —O— or —NR—;
each R^{C} is independently selected from halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R, —S(O)₂R, —S(O)N(R)₂, —S(O)₂N(R)₂, Cy^{D}, or an optionally substituted group selected from C₁₋₆ aliphatic;
each Cy^{D} is independently selected from a 3- to 7-membered saturated or partially unsaturated monocyclic carbocyclyl, a 3- to 7-membered saturated or partially unsaturated monocyclic heterocyclyl having 1-2 heteroatoms selected from oxygen, nitrogen, or sulfur, a 5- to 6-membered monocyclic heteroaryl having 1-3 heteroatoms independently selected from oxygen, nitrogen, and sulfur, phenyl, a 6- to 12-membered saturated or partially unsaturated bicyclic heterocyclyl having 1-3 heteroatoms independently selected from oxygen, nitrogen, or sulfur, a bridged bicycle, or a 6- to 12-membered saturated or partially unsaturated bicyclic spiroheterocyclyl having 1-3 heteroatoms independently selected from oxygen, nitrogen, or sulfur, wherein Cy^{D} is substituted with 0-4 -L^{D}-R^{D} groups;
each L^{D} is independently selected from a covalent bond or an optionally substituted C₁₋₆ hydrocarbon chain, wherein 1 to 3 methylene units are optionally and independently replaced with —O— or —NR—; and
each R^{D} is independently selected from oxo, halogen, —CN, —C(O)R, —C(O)₂R, —C(O)N(R)₂, —NO₂, —N(R)₂, —N(R)C(O)R, —N(R)C(O)₂R, —N(R)S(O)₂R, —OR, —OC(O)R, —OC(O)N(R)₂, —SR, —S(O)R,
—S(O)₂R, —S(O)N(R)₂, —S(O)₂N(R)₂, or an optionally substituted group selected from C₁₋₆ aliphatic, phenyl, a 3- to 7-membered saturated or partially unsaturated monocyclic carbocyclyl, or a 3- to 7-membered saturated or partially unsaturated monocyclic heterocyclyl having 1-2 heteroatoms selected from oxygen, nitrogen, or sulfur.

2. The compound of claim 1, wherein Cy^{A} is selected from the group consisting of: 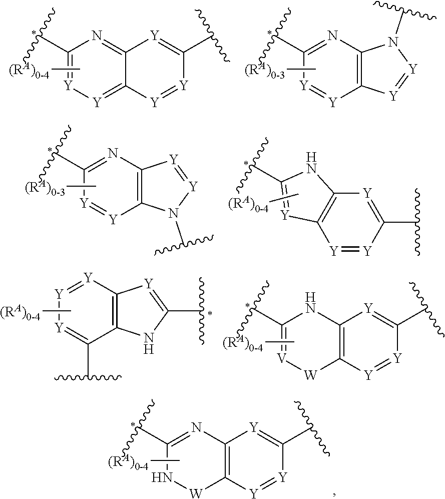
wherein:
each Y is independently selected from ═C(H)— or ═N—;
W is selected from —C(O)— or —S(O)₂—;
V is selected from ═CH— or ═N—;
and * represents point of attachment to the cyclopropyl ring.

3. The compound of claim 1, wherein Cy^{A} is selected from the group consisting of: 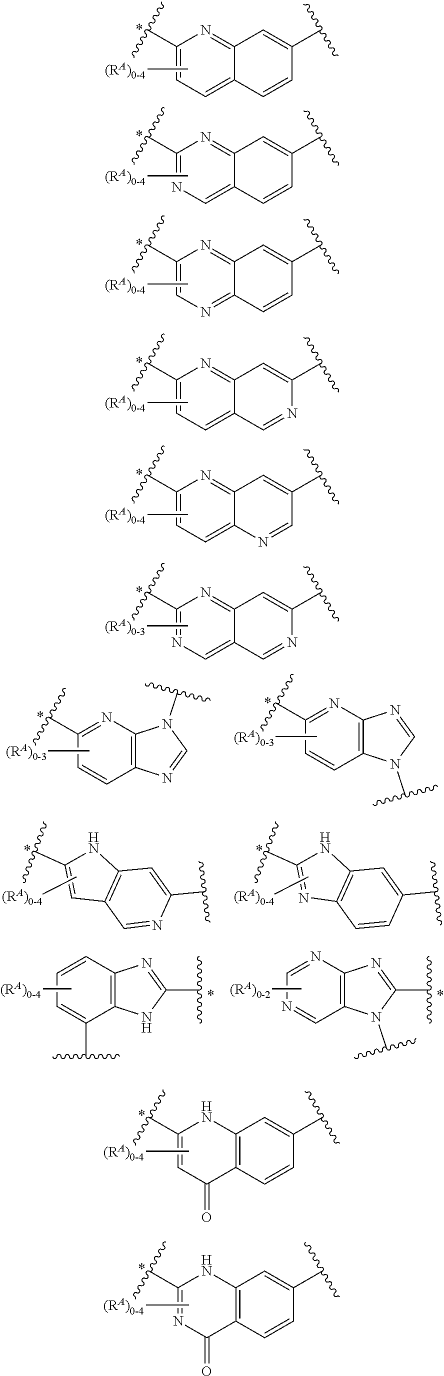 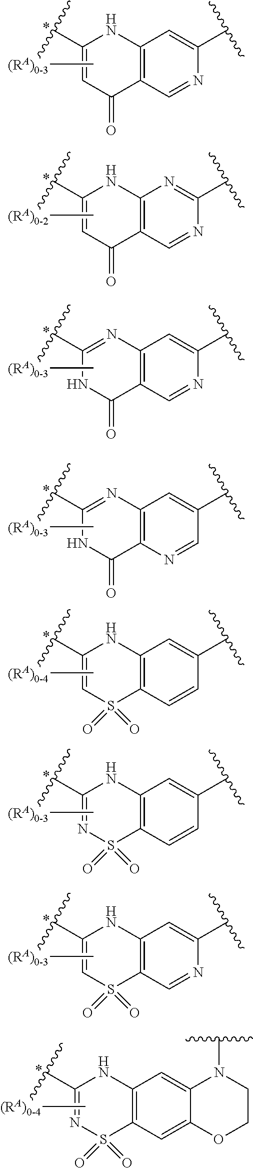
wherein * represents point of attachment to the cyclopropyl ring.

4. The compound of claim 1, wherein Cy^{A} is selected from the group consisting of: 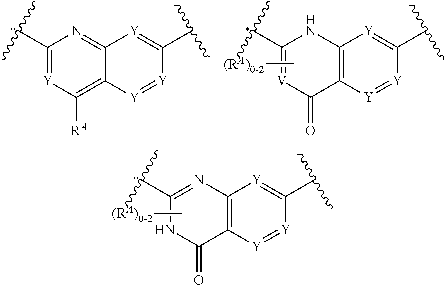
wherein:
* represents point of attachment to the cyclopropyl ring.

5. The compound of claim 1,
wherein Cy^{A} is selected from the group consisting of: 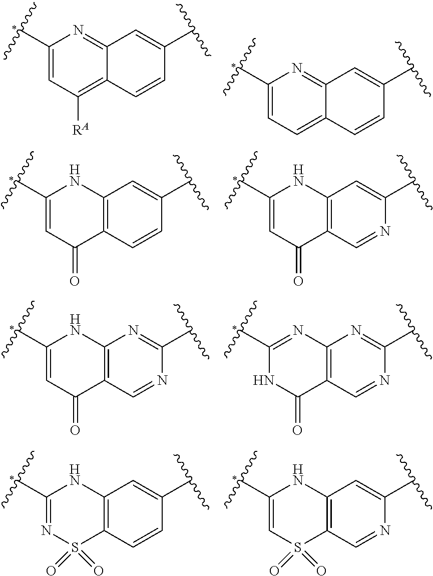
wherein * represents point of attachment to the cyclopropyl ring.

6. The compound of claim 1, wherein each R^{A} is independently selected from oxo, halogen, —CN, —N(R)₂, —N(R)S(O)₂R, —OR, or an optionally substituted group selected from C₁₋₆ aliphatic, 5- to 6-membered heteroaryl having 1-4 heteroatoms independently selected from oxygen, nitrogen, and sulfur, 3- to 7-membered saturated or partially unsaturated monocyclic heterocyclyl having 1-2 heteroatoms selected from oxygen, nitrogen, or sulfur, or a 6- to 12-membered spirocyclic ring system having 0-4 heteroatoms independently selected from oxygen, nitrogen, and sulfur.

7. The compound of claim 1, wherein Cy^{B} is selected from phenyl or a 5- to 6-membered heteroaryl having 1-3 heteroatoms independently selected from oxygen, nitrogen, and sulfur, wherein Cy^{B} is substituted with 0-4-R^{B} groups.

8. The compound of claim 1, wherein Cy^{B} is:

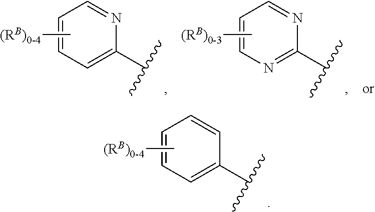

9. The compound of claim 1, wherein each R^{B} is independently selected from halogen or an optionally substituted C₁₋₆ aliphatic.

10. The compound of claim 1, wherein L is an optionally substituted C₁₋₃ hydrocarbon chain, wherein 1-3 methylene units are optionally replaced with —O—, —NR^{z}—, —S—, or —SO₂—.

11. The compound of claim 1, wherein L is *—NHCH(Me)-, *—NHCH₂—, *—OCH(Me)-, or *—OCH₂—, wherein * represents the point of attachment to Cy^{A}.

12. The compound of claim 1, wherein L is an optionally substituted 5-membered saturated or partially unsaturated heterocyclene, having 1 heteroatom independently selected from oxygen, nitrogen, and sulfur.

13. The compound of claim 1, wherein Cy^{C} is an 8- to 10-membered bicyclic aryl, wherein Cy^{C} is substituted with 0-6-L^{C}-R^{C} groups.

14. The compound of claim 1, wherein Cy^{C} is selected from the group consisting of:

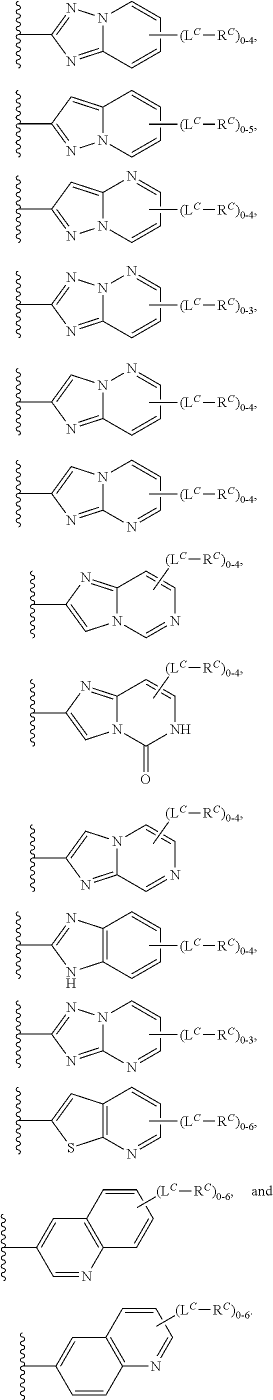

15. The compound of claim 1, wherein the compound is of Formula II: 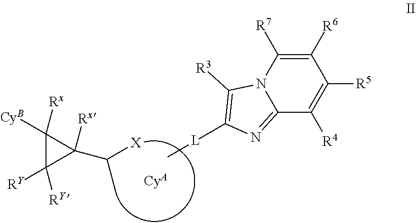
or a pharmaceutically acceptable salt thereof, wherein each R³, R⁴, R⁵, R⁶, and R⁷ is independently selected from hydrogen or -L^{C}-R^{C}.

16. The compound of claim 1, wherein each of R³, R⁴, R⁵, R⁶, and R⁷ is independently selected from hydrogen or -L^{C}-R^{C}, wherein each L^{C} is independently selected from a covalent bond or an optionally substituted C₁₋₆ hydrocarbon chain, wherein 1 to 3 methylene units are optionally and independently replaced with —O— or —NR—; and wherein each R^{C} is independently selected from halogen, —CN, —C(O)R, —C(O)₂R, —C(O)N(R)₂, —N(R)₂, —N(R)C(O)R, —N(R)C(O)₂R, —N(R)S(O)₂R, —S(O)₂R, —S(O)₂N(R)₂, Cy^{D}, or an optionally substituted group selected from C₁₋₆ aliphatic.

17. The compound of claim 1, wherein R³ is hydrogen.

18. The compound of claim 1, wherein R⁴ is selected from hydrogen or L^{C}-R^{C}, wherein L^{C} is a covalent bond and wherein R^{C} is selected from halogen, —CN, —C(O)R, —C(O)₂R, —C(O)N(R)₂, —N(R)₂, —N(R)C(O)R, —N(R)C(O)₂R, —N(R)S(O)₂R, —OR, —S(O)₂R, —S(O)₂N(R)₂, Cy^{D}, or an optionally substituted group selected from C₁₋₆ aliphatic.

19. The compound of claim 1, wherein R⁵ is hydrogen.

20. The compound of any one of the preceding claims, wherein R⁶ is selected from hydrogen or L^{C}-R^{C}, wherein L^{C} is a covalent bond, and wherein R^{C} is selected from halogen, —N(R)₂, —OR, Cy^{D}, or an optionally substituted C₁₋₆ aliphatic group.

21. The compound of claim 1, wherein R⁷ is hydrogen.

22. The compound of claim 1, wherein the compound is of Formula III: 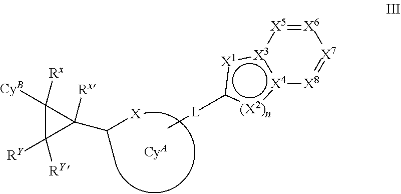
or a pharmaceutically acceptable salt thereof,
wherein X¹ is N, CH, or C-L^{C}-R^{C};
each X² is independently selected from N, CH, or C-L^{C}-R^{C};
X³ and X⁴ is independently N or C, wherein at least one of X³ or X⁴ is C;
each of X⁵, X⁶, X⁷, and X⁸ are independently selected from N, CH, or C-L^{C}-R^{C}; and
n is 1 or 2.

23. The compound of claim 1, wherein the compound is of Formulae IV-a, IV-b, or IV-c: 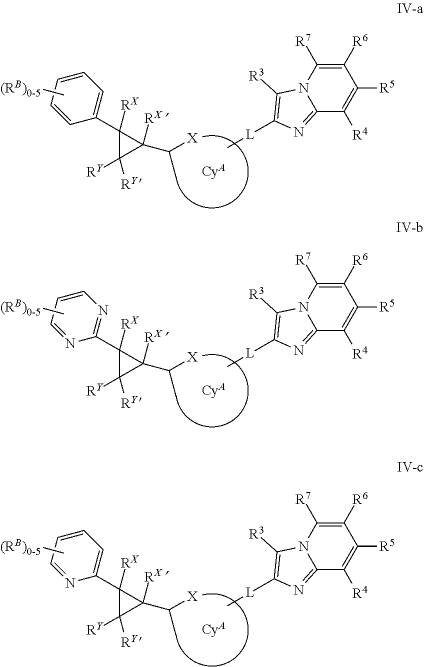
or a pharmaceutically acceptable salt thereof.

24. The compound of claim 1, wherein the compound is of Formulae V-a, V-b, or V-c: 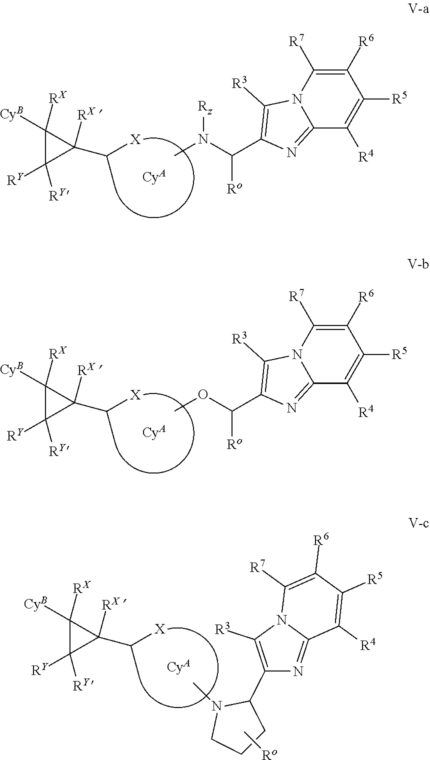
or a pharmaceutically acceptable salt thereof.

25. The compound of claim 1, wherein the compound is of Formulae VI-a, VI-b, or VI-c: 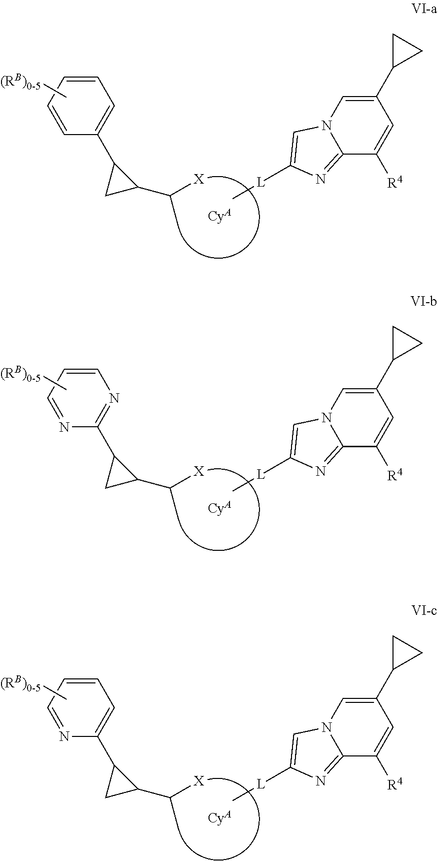
or a pharmaceutically acceptable salt thereof, and
R⁴ is hydrogen or L^{C}-R^{C}, wherein L^{C} is a covalent bond and R^{C} is halogen or Cy^{D}, wherein Cy^{D} is a 5-membered saturated or partially unsaturated monocyclic heterocyclyl having 1-2 heteroatoms selected from nitrogen, and wherein Cy^{D} is substituted with 0-4 -L^{D}-R^{D} groups.

26. The compound of claim 1, wherein Cy^{D} is a ring selected from:

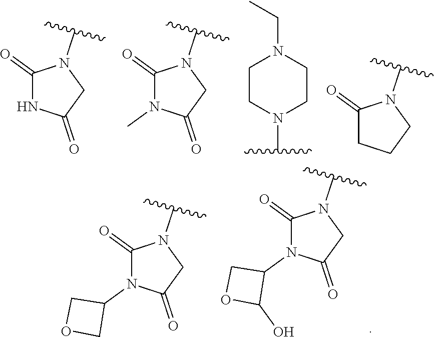

27. The compound of claim 1, wherein the compound is selected from Table A.

28. A pharmaceutical composition comprising a compound of claim 1.

29. A method of treating a plasma kallikrein-mediated disease or disorder using a compound of claim 1.

30. The method of claim 29, wherein the disease or disorder is hereditary angioedema or diabetic macular edema.
